SEQUENCE LISTING (14 sequences)

SEQ ID NO: 1 - protein, 484 aa, Artificial sequence
  Synthetic construct
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 2 - protein, 482 aa, Artificial sequence
  Synthetic construct
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKAGADGTEFVDAVEVDPSNRNQETSGTYQIQAWTKFDFPGRGNTYSSFKWRWYHFDGTDWDESRKLNRIYKFTGKAWDWEVDTENGNYDYLMFADLDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNGGSVSIWVKR

SEQ ID NO: 3 - protein, 483 aa, Bacillus species
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQDKAGQVWHDITGNKPGTVTINADGWANFSVNGGSVSIWVKR

SEQ ID NO: 4 - protein, 485 aa, Bacillus species
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQDKAGQVWHDITGNKPGTVTINADGWANFSVNGGSVSIWVKR

SEQ ID NO: 5 - protein, 514 aa, Bacillius species
MKQQKRLYARLLTLLFALIFLLPHSAAAAHHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAWTKFDFPGRGNTYSDFKWRWYHFDGVDWDQSRQFQNRIYKFRGDGKAWDWEVDSENGNYDYLMYADVDMDHPEVVNELRRWGEWYTNTLNLDGFRIDAVKHIKYSFTRDWLTHVRNATGKEMFAVAEFWKNDLGALENYLNKTNWNHSVFDVPLHYNLYNASNSGGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQDKAGQVWHDITGNKPGTVTINADGWANFSVNGGSVSIWVKR

SEQ ID NO: 6 - protein, 485 aa, Bacillus species
HHNGTNGTMMQYFEWYLPNDGNHWNRLRSDASNLKDKGISAVWIPPAWKGASQNDVGYGAYDLYDLGEFNQKGTIRTKYGTRNQLQAAVNALKSNGIQVYGDVVMNHKGGADATEMVRAVEVNPNNRNQEVSGEYTIEAWTKFDFPGRGNTHSNFKWRWYHFDGVDWDQSRKLNNRIYKFRGDGKGWDWEVDTENGNYDYLMYADIDMDHPEVVNELRNWGVWYTNTLGLDGFRIDAVKHIKYSFTRDWINHVRSATGKNMFAVAEFWKNDLGAIENYLNKTNWNHSVFDVPLHYNLYNASKSGGNYDMRQIFNGTVVQRHPMHAVTFVDNHDSQPEEALESFVEEWFKPLAYALTLTREQGYPSVFYGDYYGIPTHGVPAMKSKIDPILEARQKYAYGRQNDYLDHHNIIGWTREGNTAHPNSGLATIMSDGAGGNKWMFVGRNKAGQVWTDITGNRAGTVTINADGWGNFSVNGGSVSIWVNK

SEQ ID NO: 7 - protein, 485 aa, Bacillus species
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGKQNDYLDHHNIIGWTREGNTAHPNSGLATIMSDGAGGSKWMFVGRNKAGQVWSDITGNRTGTVTINADGWGNFSVNGGSVSIWVNK

SEQ ID NO: 8 - protein, 485 aa, Bacillus species
HHNGTNGTMMQYFEWYLPNDGNHWNRLRSDASNLKDKGITAVWIPPAWKGASQNDVGYGAYDLYDLGEFNQKGTVRTKYGTRNQLQAAVTALKSNGIQVYGDVVMNHKGGADATEWVRAVEVNPSNRNQEVSGDYTIEAWTKFDFPGRGNTHSNFKWRWYHFDGVDWDQSRQLQNRIYKFRGDGKGWDWEVDTENGNYDYLMYADIDMDHPEVVNELRNWGVWYTNTLGLDGFRIDAVKHIKYSFTRDWLTHVRNTTGKNMFAVAEFWKNDIGAIENYLSKTNWNHSVFDVPLHYNLYNASRSGGNYDMRQIFNGTVVQRHPTHAVTFVDNHDSQPEEALESFVEEWFKPLACALTLTRDQGYPSVFYGDYYGIPTHGVPAMKSKIDPILEARQKYAYGKQNDYLDHHNMIGWTREGNTAHPNSGLATIMSDGPGGNKWMYVGRNKAGQVWRDITGNRSGTVTINADGWGNFSVNGGSVSIWVNN

SEQ ID NO: 9 - protein, 483 aa, Bacillus licheniformis [sequence omitted]

SEQ ID NO: 10 - protein, 613 aa, Bacillus species
MKQQKRLYARLLTLLFALIFLLPHSAAAAANTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAKTSNVTFTVNNATTTSGQNVYVVANIPELGNWNTANAIKMNPSSYPTWKATIALPQGKAIEFKFIKKDQAGNVIWESTSNRTYTVPFSSTGSYTASWNVP

SEQ ID NO: 11 - protein, 582 aa, Bacillus species
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDALHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAKTSNVTFTVNNATTTSGQNVYVVANIPELGNWNTANAIKMNPSSYPTWKATIALPQGKAIEFKFIKKDQAGNVIWESTSNRTYTVPFSSTGSYTASWNVP

SEQ ID NO: 12 - protein, 484 aa, Bacillus species
NTAPINETMMQYFEWDLPNDGTLWTKVKNEAANLSSLGITALWLPPAYKGTSQSDVGYGVYDLYDLGEFNQKGTIRTKYGTKTQYIQAIQAAKAAGMQVYADVVFNHKAGADGTEFVDAVEVDPSNRNQETSGTYQIQAWTKFDFPGRGNTYSSFKWRWYHFDGTDWDESRKLNRIYKFRSTGKAWDWEVDTENGNYDYLMFADLDMDHPEVVTELKNWGTWYVNTTNIDGFRLDAVKHIKYSFFPDWLTYVRNQTGKNLFAVGEFWSYDVNKLHNYITKTNGSMSLFDAPLHNNFYTASKSSGYFDMRYLLNNTLMKDQPSLAVTLVDNHDTQPGQSLQSWVEPWFKPLAYAFILTRQEGYPCVFYGDYYGIPKYNIPGLKSKIDPLLIARRDYAYGTQRDYIDHQDIIGWTREGIDTKPNSGLAALITDGPGGSKWMYVGKKHAGKVFYDLTGNRSDTVTINADGWGEFKVNGGSVSIWVAK

SEQ ID NO: 13 - protein, 483 aa, Bacillus halmapalus
HHNGTNGTMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKGGADATENVLAVEVNPNNRNQEISGDYTIEAYTKFDFPGRGNTYSDFKWRWYHFDGVDWDQSRQFQNRIYKFRGKAWDWEVDSEFGNYDYLMYADYDMDHPEVVNELRRWGEWYTNTLNLDGFRIDAVKHIKFSFTRDWLTHVRNATGKGMFAVAEFWKNDLGALENYLNKTNWNHSVFDVPLHYNLYNASNSRGNYDMAKLLNGTVVQKHPMHAVTFVDNHDSQPGESLESFVQEWFKPLAYALILTREQGYPSVFYGDYYGIPTHSVPAMKAKIDPILEARQNFAYGTQHDYFDHHNIIGWTREGNTTHPNSGLATIMSDGPGGEKWMYVGQNKAGQVWHDITGNKPGTVTINADGWANFSVNKGSVSIWVKR

SEQ ID NO: 14 - protein, 482 aa, Bacillus halmapalus
HNGTNATMMQYFEWHLPNDGNHWNRLRDDASNLRNRGITAIWIPPAWKGTSQNDVGYGAYDLYDLGEFNQKGTVRTKYGTRSQLESAIHALKNNGVQVYGDVVMNHKAGADATENVLAVEVNPNNRNQEISGDYTIEAYTKFDFPGRGNTYSDFKWRWYHFDGVDWDQSRQFQNRIYKFRGKAWDWEVDSEFGNYDYLMYADYDMDHPEVVNELRRWGEWYTNTLNLDGFRIDAVKHIKFSFTRDWLTHVRNATGKGMFAVAEFWKNDLGALENYLSKTNWNHSVFDVPLHYNLYNASNSRGNYDMAKLLNGTVVQKHPMHAVTFVDNHDSQPGESLESFVQEWFKPLAYALILTREQGYPSVFYGDYYGIPTHSVPAMKAKIDPILAARQNFAYGTQHDYFDHHNIIGWTREGNTTHPNSGLATIMSDGPGGEKWMYVGQNKAGQVWHDITGNKPGTVTINADGWANFSVNKGSVSIWVKR

(19) United States
(12) Patent Application Publication
(10) Pub. No.: US 2023/0118258 A1
(43) Pub. Date: Apr. 20, 2023

(54) Alpha-Amylase Variants

(71) Applicant: Novozymes A/S, Bagsvaerd (DK)

(72) Inventors: Carsten Andersen, Vaerloese (DK); Rajendra Kulotungan Sainathan, Bangalore (IN); Madhupriya Mahankali, Hyderabad (DK); Subith Krishna, Bangalore (DK); Dinesh Prasad, Pradesh (IN)

(73) Assignee: Novozymes A/S, Bagsvaerd (DK)

(21) Appl. No.: 17/781,512

(22) PCT Filed: Dec. 18, 2020
(86) PCT No.: PCT/EP2020/087043

§ 371 Date: Jun. 1, 2022

(30) Foreign Application Priority Data
Dec. 19, 2019 (IN) 201941052843
Dec. 9, 2020 (IN) 202041053525

(51) Int. Cl. C12N 9/28; C11D 3/386

(52) U.S. Cl. CPC C12N 9/2417; C12Y 302/01001; C11D 3/386

(57) ABSTRACT

The present invention relates to alpha-amylase variants of a parent alpha-amylase having an improved wash performance when compared to the parent alpha-amylase. The present invention also relates to polynucleotides encoding the variants, nucleic acid constructs, vectors, and host cells comprising the polynucleotides, and method of producing the variants of the present invention.

### REFERENCE TO A SEQUENCE LISTING

This application contains a Sequence Listing in computer readable form. The computer readable form is incorporated herein by reference.

### FIELD OF THE INVENTION

The present invention relates to alpha-amylase variants having alpha-amylase activity. The present invention further relates to polynucleotides encoding such variants, vectors and host cells comprising genes encoding such variants, which may also enable the production of such variants. The present invention further relates to use in household cleaning or treatment, in particular laundry and dishwashing, including hand wash and/or automatic laundry and/or hand wash and/or automatic dishwashing. The present invention is particularly useful for cleaning laundry.

### BACKGROUND OF THE INVENTION

Alpha-amylases (alpha-1,4-glucan-4-glucanohydrolases, E.C. 3.2.1.1) constitute a group of enzymes, which catalyse hydrolysis of starch and other linear and branched 1,4-glucosidic oligo- and polysaccharides. They have been known for use in household for many years, including the first bacterial alpha-amylases to be used from B. licheniformis, also known as Termamyl. Alkaline amylases, for example those from AA560 (WO2000/060060) and from SP707 (described by Tsukamoto et al., 1988, Biochem. Biophys. Res. Comm. 151: 25-31) are useful in detergents and it is known to modify these and other amylases to improve their stability in detergents. For example, WO 96/23873 discloses to delete the amino acids 181+182 or the amino acids 183+184 of SP707 (SEQ ID NO: 7 of WO 96/23873) to improve the stability of this amylase. WO 96/23873 further discloses to modify the SP707 amylase by substituting M202 with e.g. a leucine to stabilize the molecule towards oxidation.

It is a constant challenge to improve the performance of amylolytic enzymes, especially in an environmentally favourable way. For environmental reasons it is also increasingly important to provide amylolytic enzymes that are effective, preferably also in quick wash cycles often using smaller volumes of water and/or lower temperatures. Thus, it is desirable to have amylolytic enzymes that exhibit an improved property, such as specific activity, when compared to the parent polypeptide. The present invention provides variant polypeptides having alpha-amylase activity and an improved property or enzyme detergency benefit, such as removal of starch-containing stains, or specific activity, compared to its hybrid and/or a parent polypeptide. Particularly preferred amylolytic enzymes can function under low temperature and/or in shorter washing cycles and at the same time preserve or increase other desirable properties such as specific activity (amylolytic activity), stability and/or wash performance.

Thus, it is an object of the present invention to provide alpha-amylase variants exhibiting an improved property, such as removal of starch-containing stains or specific activity, when compared to the parent polypeptide and which can be used in washing, dishwashing and/or cleaning processes at low temperature, such as temperatures of 5-40° C. It is a further object of the present invention to provide alpha-amylase variants which have improved wash performance compared to the parent alpha-amylase or compared to the alpha-amylase of any of SEQ ID NOs: 1, 2, 3, 4, 5, 6, 7, 8, 9, 10, 11, 12, 13 or 14.

### SUMMARY OF THE INVENTION

The present invention relates to an alpha-amylase variant of a parent alpha-amylase comprising a modification at one or more positions corresponding to position: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO. 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the parent polypeptide.

The present invention relates to an alpha-amylase variant of a parent alpha-amylase comprising a modification at one or more positions corresponding to position: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO. 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the parent polypeptide and wherein said variant has an improved wash performance, wherein the improved wash performance compared to parent polypeptide, wherein the parent polypeptide is preferably SEQ ID NO: 1 or SEQ ID NO: 2 or SEQ ID NO: 3.

The present invention also relates to a polynucleotide encoding an alpha amylase variant according to the invention, a nucleic acid construct comprising the polynucleotide encoding the variant according to the invention, an expression vector comprising the polynucleotide encoding the variant according to the invention, or a host cell comprising the polynucleotide encoding the variant according to the invention.

The present invention also relates to a method of producing an alpha-amylase variant, comprising (a) cultivating the host cell of the invention under conditions suitable for expression of the variant, and (b) recovering the variant.

The present invention further relates to a method of obtaining an alpha-amylase variant, comprising introducing into a parent alpha-amylase a modification at one or more positions corresponding to position 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO. 1 for numbering, wherein each modification is independently a substitution or deletion, and the variant has alpha-amylase activity; and recovering the variant.

### Definitions

In accordance with this detailed description, the following definitions apply. Note that the singular forms “a,” “an,” and “the” include plural references unless the context clearly dictates otherwise.

Reference to “about” a value or parameter herein includes aspects that are directed to that value or parameter per se. For example, description referring to “about X” includes the aspect “X”.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1 percent to about 5 percent” or “about 0.1 percent to 5 percent” should be interpreted to include not just about 0.1 percent to about 5 percent, but also the individual values (e.g., 1 percent, 2 percent, 3 percent, and 4 percent) and the sub-ranges (e.g., 0.1 percent to 0.5 percent, 1.1 percent to 2.2 percent, 3.3 percent to 4.4 percent) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless defined otherwise or clearly indicated by context, all technical and scientific terms used herein have the same meaning as commonly understood by one of ordinary skill in the art to which this invention belongs.

A-, B- and C-domains: The structure of alpha-amylases comprises three distinct domains A, B and C, see, e.g., Machius et al., 1995, J. Mol. Biol. 246: 545-559. The term “domain” means a region of a polypeptide that forms a distinct and independent substructure of the whole molecule. Alpha-amylases consist of a beta/alpha-8 barrel harboring the active site, which is denoted the A-domain, a rather long loop between the beta-sheet 3 and alpha-helix 3, which is denoted the B-domain, and a C-domain and in some cases also a carbohydrate binding domain (e.g., WO 2005/001064; Machius et al., supra).

The domains of an alpha-amylase can be determined by structure analysis such as by using crystallographically techniques. An alternative method for determining the domains of an alpha-amylase is by sequence alignment of the amino acid sequence of the alpha-amylase with another alpha-amylase for which the domains have been determined. The sequence that aligns with, e.g., the B-domain sequence in the alpha-amylase for which the B-domain has been determined can be considered the B-domain for the given alpha-amylase.

Allelic variant: The term “allelic variant” means any of two or more alternative forms of a gene occupying the same chromosomal locus. Allelic variation arises naturally through mutation and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Alpha-amylases: The term “alpha-amylase” is synonymous with the term “polypeptides having alpha-amylase activity”. “Alpha-amylase activity” means the activity of alpha-1,4-glucan-4-glucanohydrolases, E.C. 3.2.1.1, which constitute a group of enzymes, catalyzing hydrolysis of starch and other linear and branched 1,4-glucosidic oligo- and polysaccharides. Thus, the term “alpha-amylase” as used herein, refers to an enzyme that has alpha-amylase activity (Enzyme Class; EC 3.2.1.1) that hydrolyses alpha bonds of large, alpha-linked polysaccharides, such as starch and glycogen, yielding glucose and maltose. The terms “alpha-amylase” and “amylase” may be used interchangeably and constitute the same meaning and purpose herein. For purposes of the present invention, alpha-amylase activity is determined according to the procedure described under the sub-heading Methods, below. In one aspect, the alpha-amylases of the present invention have at least 20%, e.g., at least 40%, at least 50%, at least 60%, such as at least 65%, such as at least 70%, such as at least 75%, such as at least 80%, such as about 85%, about 90%, about 91%, about 92%, about 93%, about 94%, about 95%, about 96%, about 97%, about 98%, about 99%, but less than 100% sequence identity to the amino acid sequence to the polypeptide of SEQ ID No: 1, 2, 3, 4, 5, 6, 7, 8, 9, 10, 11, 12, 13 or 14

Amino acid: The term ‘amino acid’ as used herein, refers to the standard twenty genetically-encoded amino acids and their corresponding stereoisomers in the ‘d’ form (as compared to the natural ‘l’ form), omega-amino acids other naturally-occurring amino acids, unconventional amino acids (e.g. α, α-disubstituted amino acids, N-alkyl amino acids, etc.) and chemically derivatised amino acids. Chemical derivatives of one or more amino acids may be achieved by reaction with a functional side group. Such derivatised molecules include, for example, those molecules in which free amino groups have been derivatised to form amine hydrochlorides, p-toluene sulphonyl groups, carboxybenzoxy groups, t-butyloxycarbonyl groups, chloroacetyl groups or formyl groups. Free carboxyl groups may be derivatised to form salts, methyl and ethyl esters or other types of esters and hydrazides. Free hydroxyl groups may be derivatised to form O-acyl or O-alkyl derivatives. Also included as chemical derivatives are those peptides which contain naturally occurring amino acid derivatives of the twenty standard amino acids. For example: 4-hydroxyproline may be substituted for proline; 5-hydroxylysine may be substituted for lysine; 3-methylhistidine may be substituted for histidine; homoserine may be substituted for serine and ornithine for lysine. Derivatives also include peptides containing one or more additions or deletions as long as the requisite activity is maintained. Other included modifications are amidation, amino terminal acylation (e.g. acetylation or thioglycolic acid amidation), terminal carboxylamidation (e.g. with ammonia or methylamine), and the like terminal modifications.

When an amino acid is being specifically enumerated, such as ‘alanine’ or ‘Ala’ or ‘A’, the term refers to both l-alanine and d-alanine unless explicitly stated otherwise. Other unconventional amino acids may also be suitable components for polypeptides of the present invention, as long as the desired functional property is retained by the polypeptide. For the peptides shown, each encoded amino acid residue, where appropriate, is represented by a single letter designation, corresponding to the trivial name of the conventional amino acid. In one embodiment, the polypeptides of the invention comprise or consist of I-amino acids.

Catalytic domain: The term “catalytic domain” means the region of an enzyme containing the catalytic machinery of the enzyme.

cDNA: The term “cDNA” means a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a variant. The boundaries of the coding sequence are generally determined by an open reading frame, which usually begins with a start codon such as ATG, GTG or TTG and ends with a stop codon such as TAA, TAG, or TGA. The coding sequence may be a DNA, cDNA, synthetic, or recombinant polynucleotide.

Control sequences: The term “control sequences” means nucleic acid sequences necessary for the expression of a polynucleotide encoding a variant of the present invention. Each control sequence may be native (i.e., from the same gene) or foreign (i.e., from a different gene) to the polynucleotide encoding the variant or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Corresponding to: The term “corresponding to” as used herein, refers to a way of determining the specific amino acid of a sequence wherein reference is made to a specific amino acid sequence. E.g. for the purposes of the present invention, when references are made to specific amino acid positions, the skilled person would be able to align another amino acid sequence to said amino acid sequence that reference has been made to, in order to determine which specific amino acid may be of interest in said another amino acid sequence. Alignment of another amino acid sequence with e.g. the sequence as set forth in SEQ ID No: 1, 2, 3, 4, 5, 6, 7, 8, 9, 10, 11, 12, 13 or 14, or any other sequence listed herein, has been described elsewhere herein. Alternative alignment methods may be used and are well-known for the skilled person.

Dish-washing composition: The term “dish-washing composition” as used herein, refers to all forms of compositions for cleaning dishes. The dish-washing composition is a liquid dish washing composition, a powder dish washing composition optionally wherein the composition is in the form of a unit dose. A unit dose dish-washing composition may comprise more than one compartment, the multiple compartments comprising powder, liquid or a combination thereof.

Enzyme Detergency Benefit: The term “enzyme detergency benefit” used herein, refers to the advantageous effect an enzyme may add to a detergent compared to the same detergent without the enzyme. Important detergency benefits which can be provided by enzymes are stain removal with no or very little visible soils after washing and/or cleaning, prevention or reduction of re-deposition of soils released in the washing process (an effect that also is termed anti-redeposition), restoring fully or partly the whiteness of textiles which originally were white but after repeated use and wash have obtained a greyish or yellowish appearance (an effect that also is termed whitening). Textile care benefits, which are not directly related to catalytic stain removal or prevention of re-deposition of soils, may also be important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric-softness, colour clarification of the fabric and removal of particulate soils which are trapped in the fibers of the fabric or garment. Enzymatic bleaching is a further enzyme detergency benefit where the catalytic activity generally is used to catalyze the formation of bleaching component such as hydrogen peroxide or other peroxides.

Expression: The term “expression” includes any step involved in the production of the variant including, but not limited to, transcription, post-transcriptional modification, translation, post-translational modification, and secretion.

Expression vector: The term “expression vector” means a linear or circular DNA molecule that comprises a polynucleotide encoding a variant and is operably linked to additional nucleotides that provide for its expression.

Fragment: The term “fragment” as used herein, refers to a polypeptide having one or more (e.g., several) amino acids absent from the amino and/or carboxyl terminus of the mature polypeptide of any one of the parent sequences herein disclosed, such as SEQ ID NOs: 1-14; wherein the fragment has alpha-amylase activity. In one aspect, a fragment contains at least 200 contiguous amino acid residues of SEQ ID NOs: 1-14, for example at least 300 contiguous amino acid residues, or at least 350 contiguous amino acid residues, or at least 400 contiguous amino acid residues, or at least 450 contiguous amino acid residues of SEQ ID NOs: 1-14.

Fusion Polypeptide: The term “fusion polypeptide” as used herein, refers to a polypeptide which comprises amino acid sequences originating from more than one, such as two, three, or four, species. Such a fusion polypeptide may have been generated by molecular techniques well-known to the skilled person. A fusion polypeptide of the present invention has alpha-amylase activity. In particular, a fusion polypeptide of the present invention, comprises e.g. an A and C domain of one alpha-amylase and a B domain from another alpha-amylase. Hybrid polypeptide (or “hybrid”) and fusion polypeptide may be used interchangeably and constitute the same meaning and purpose.

Hard surface cleaning: The term “hard surface cleaning” as used herein, refers to cleaning of any hard surfaces including floors, roofs, cars, kitchen or bathroom surfaces, as well as dish cleaning (dish-washing).

Heterologous polynucleotide: The term “heterologous polynucleotide” is defined herein as a polynucleotide that is not native to the host cell; a native polynucleotide in which structural modifications have been made to the coding region; a native polynucleotide whose expression is quantitatively altered as a result of a manipulation of the DNA by recombinant DNA techniques, e.g., a different (foreign) promoter; or a native polynucleotide in a host cell having one or more extra copies of the polynucleotide to quantitatively alter expression. A “heterologous gene” is a gene comprising a heterologous polynucleotide.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, or domain of a polypeptide or nucleic acid is not naturally associated with the polypeptide or nucleic acid, i.e., the control sequence is from a gene other than the gene encoding the variants.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Host cell: The term “host cell” means any microbial or plant cell into which a nucleic acid construct or expression vector comprising a polynucleotide of the present invention has been introduced. Methods for introduction include but are not limited to protoplast fusion, transfection, transformation, electroporation, conjugation, and transduction. In some embodiments, the host cell is an isolated recombinant host cell that is partially or completely separated from at least one other component with, including but not limited to, proteins, nucleic acids, cells, etc.

Hybridization: The term “hybridization” means the pairing of substantially complementary strands of nucleic acids, using standard Southern blotting procedures. Hybridization may be performed under medium, medium-high, high or very high stringency conditions. Medium stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 55° C. Medium-high stringency conditions means prehybridization and hybridization at 42° C. in 5×SSPE, 0.3% SDS, 200 micrograms/ml sheared and denatured salmon sperm DNA, and 35% formamide for 12 to 24 hours, followed by washing three times each for 15 minutes using 0.2×SSC, 0.2% SDS at 60° C. High stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 65° C. Very high stringency conditions means prehybridization and hybridization at 42° C. in 5×SSPE, 0.3% SDS, 200 micrograms/ml sheared and denatured salmon sperm DNA, and 50% formamide for 12 to 24 hours, followed by washing three times each for 15 minutes using 0.2×SSC, 0.2% SDS at 70° C.

Improved property: The term “improved property” is defined herein as a characteristic associated with a variant that is improved compared to the parent alpha-amylase. Such improved properties include, but are not limited to, increased amylolytic activity, increased catalytic efficiency, increased catalytic rate, increased chemical stability, increased oxidation stability, increased pH activity, increased pH stability, increased specific activity, increased substrate binding, increased substrate cleavage, increased substrate specificity, increased substrate stability, increased surface properties, increased thermal activity, increased thermostability, increased wash performance such as soil removal performance e.g. performance to starch-containing soils, stain removal, anti-greying, stability e.g. thermostability, pH stability, or stability in the presence of builders, including chelant, stability in powder, liquid or gel detergent formulation or dishwashing composition, altered temperature-dependent performance and activity profile, pH activity, substrate specificity, product specificity, and chemical stability.

Improved wash performance: The term “improved wash performance” is defined herein as displaying an improvement of the wash performance of an amylase relative to the wash performance of the parent amylase or relative to the alpha-amylase of any of SEQ ID NO: 1, SEQ ID NO: 2 or SEQ ID NO: 3 e.g. by increased stain removal. Improved wash performance may be measured by comparing of the so-called Intensity value. Preferably improved wash performance is determined according to the Examples. The wash performance is improved if the Improvement Factor (IF) is above 1.0, preferably above 1.05 in one or more of the conditions listed in the examples. The wash conditions are described in the Example section. The term “wash performance” includes cleaning in general e.g. hard surface cleaning as in dish wash, but also wash performance on textiles such as laundry, and also industrial and institutional cleaning. Improved wash performance may be measured by comparing the delta intensities as described in the definition herein.

Isolated: The term “isolated” means a substance in a form or environment which does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample. In one aspect, the present invention relates to an isolated alpha-amylase variant.

Intensity value: The wash performance is measured as the brightness expressed as the intensity of the light reflected from the sample when illuminated with white light. When the sample is stained the intensity of the reflected light is lower, than that of a clean sample. Therefore, the intensity of the reflected light can be used to measure wash performance, where a higher intensity value correlates with higher wash performance. Color measurements are made with a professional flatbed scanner (Kodak iQsmart, Kodak) used to capture an image of the washed textile. To extract a value for the light intensity from the scanned images, 24-bit pixel values from the image are converted into values for red, green and blue (RGB). The intensity value (Int) is calculated by adding the RGB values together as vectors and then taking the length of the resulting vector:

Int=\sqrt{r²+g²+b²}

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 0.2×SSC, 0.2% SDS at 50° C.

Low temperature: “Low temperature” is a temperature of 5-40° C., or 5-35° C., or 5-30° C., or even 5-25° C., or 5-20° C., or 5-15° C., or 5-10° C. Preferably, “Low temperature” is a temperature of 10-35° C., preferably 10-30° C., or 10-25° C., or 10-20° C.

Mature polypeptide coding sequence: The term “mature polypeptide coding sequence” means a polynucleotide that encodes a mature polypeptide having alpha-amylase activity.

Mature polypeptide: The term “mature polypeptide” means a polypeptide in its final form following translation and any post-translational modifications, such as N-terminal processing, C-terminal truncation, glycosylation, phosphorylation, etc. It is known in the art that a host cell may produce a mixture of two of more different mature polypeptides (i.e., with a different C-terminal and/or N-terminal amino acid) expressed by the same polynucleotide.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 60° C.

Modification: The term “modification”, in the context of the polypeptides of the invention, means that one or more amino acids within the reference amino acid sequence (e.g. SEQ ID NOs: 1, 2, 3, 4, 5, 6, 7, 8, 9, 10, 11, 12, 13 or 14) are altered by substitution with a different amino acid, by insertion of an amino acid or by deletion, preferably by at least one deletion. The terms “modification”, “alteration”, and “mutation” may be used interchangeably and constitute the same meaning and purpose.

Mutant: The term “mutant” means a polynucleotide encoding a variant.

Native: The term “native” means a nucleic acid or polypeptide naturally occurring in a host cell.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences. The term nucleic acid construct is synonymous with the term “expression cassette” when the nucleic acid construct contains the control sequences required for expression of a coding sequence of the present invention.

Operably linked: The term “operably linked” means a configuration in which a control sequence is placed at an appropriate position relative to the coding sequence of a polynucleotide such that the control sequence directs the expression of the coding sequence.

Parent or Parent alpha-amylase: The term “parent” alpha-amylase as used herein means an alpha-amylase to which modifications are made to produce the variant alpha-amylase of the present invention. This term also refers to the polypeptide with which a variant of the invention is compared. The parent may be a naturally occurring (wild type) polypeptide, or it may even be a variant thereof, prepared by any suitable means. For instance, the parent protein may be a variant of a naturally occurring polypeptide which has been modified or altered in the amino acid sequence. Thus, the parent alpha-amylase may have one or more (or one or several) amino acid substitutions, deletions and/or insertions. Thus, the parent alpha-amylase may be a variant of a parent alpha-amylase. A parent may also be an allelic variant which is a polypeptide encoded by any of two or more alternative forms of a gene occupying the same chromosomal locus. The term “parent” or “parent alpha-amylase” as used herein, refers to the alpha-amylase of SEQ ID NOs: SEQ ID NOs: 1, 2, 3, 4, 5, 6, 7, 8, 9, 10, 11, 12, 13 or 14, or any alpha-amylase having at least 60% sequence identity to any of the polypeptides of SEQ ID NOs: 1, 2, 3, 4, 5, 6, 7, 8, 9, 10, 11, 12, 13 or 14. The parent alpha-amylase may also be a polypeptide comprising a fragment of SEQ ID NOs: 1-14. The parent may be a bacterial or a fungal alpha-amylase, preferably a bacterial alpha-amylase. The amylase having SEQ ID NO: 1, 2 or 3 may for example be a parent or hybrid polypeptide essential to the present invention.

Purified: The term “purified” means a nucleic acid or polypeptide that is substantially free from other components as determined by analytical techniques well known in the art (e.g., a purified polypeptide or nucleic acid may form a discrete band in an electrophoretic gel, chromatographic eluate, and/or a media subjected to density gradient centrifugation). A purified nucleic acid or polypeptide is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight on a molar basis). In a related sense, a composition is enriched for a molecule when there is a substantial increase in the concentration of the molecule after application of a purification or enrichment technique. The term “enriched” refers to a compound, polypeptide, cell, nucleic acid, amino acid, or other specified material or component that is present in a composition at a relative or absolute concentration that is higher than a starting composition.

Recombinant: The term “recombinant,” when used in reference to a cell, nucleic acid, protein or vector, means that it has been modified from its native state. Thus, for example, recombinant cells express genes that are not found within the native (non-recombinant) form of the cell, or express native genes at different levels or under different conditions than found in nature. Recombinant nucleic acids differ from a native sequence by one or more nucleotides and/or are operably linked to heterologous sequences, e.g., a heterologous promoter in an expression vector. Recombinant proteins may differ from a native sequence by one or more amino acids and/or are fused with heterologous sequences. A vector comprising a nucleic acid encoding a polypeptide is a recombinant vector. The term “recombinant” is synonymous with “genetically modified” and “transgenic”.

Sequence Identity: The relatedness between two amino acid sequences or between two nucleotide sequences is described by the parameter “sequence identity”.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used may be gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

Signal peptide: The term “signal peptide” is defined herein as a peptide linked (fused) in frame to the amino terminus of a polypeptide having biological activity and directs the polypeptide into the cell's secretory pathway. Signal sequences may be determined using techniques known in the art (See, e.g., Zhang and Henzel, 2004, Protein Science 13: 2819-2824).

Starch binding domain: The terms “starch binding domain (SBD) or carbohydrate binding module (CBM)” are used interchangeably herein. SBDs can be divided into nine CBM families. As a source of energy, starch is degraded by a large number of various amylolytic enzymes. However, only about 10 percent of them are capable of binding and degrading raw starch. These enzymes usually possess a distinct sequence-structural module called the starch-binding domain that mediates attachment to starch granules. SBD refers to an amino acid sequence that binds preferentially to a starch (polysaccharide) substrate or a maltosaccharide, alpha-, beta and gamma-cyclodextrin and the like. They are usually motifs of approximately 100 amino acid residues found in about 10 percent of microbial amylolytic enzymes.

Subsequence: The term “subsequence” means a polynucleotide having one or more (e.g. several) nucleotides deleted from the 5′- and/or 3′-end of a mature polypeptide coding sequence; wherein the subsequence encodes a fragment having alpha-amylase activity.

Textile Care Benefits: The term “textile care benefits”, as used herein, is defined as not being directly related to catalytic stain removal or prevention of re-deposition of soils, are also important for enzyme detergency benefits. Examples of such textile care benefits are prevention or reduction of dye transfer from one textile to another textile or another part of the same textile (dye transfer inhibition), removal of protruding or broken fibers from a textile surface to decrease pilling tendencies or remove already existing pills or fuzz (anti-pilling), improvement of the textile-softness, color clarification of the textile and removal of particulate soils which are trapped in the fibers of the textile. Enzymatic bleaching is a further enzyme detergency benefit where the catalytic activity generally is used to catalyze the formation of bleaching component such as hydrogen peroxide or other peroxides or other bleaching species.

Variant: The term “variant” as used herein means a polypeptide having alpha-amylase activity comprising a modification, i.e., a substitution, insertion, and/or deletion, at one or more (e.g. several) positions. A substitution means replacement of an amino acid occupying a position with a different amino acid; a deletion means removal of an amino acid occupying a position; and an insertion means adding an amino acid adjacent to and immediately following an amino acid occupying a position. The variant have at least 20%, e.g., at least 40%, at least 50%, at least 60%, at least 65%, at least 70%, at least 75%, at least 80%, about 85%, about 90%, about 91%, about 92%, about 93%, about 94%, about 95%, about 96%, about 97%, about 98%, about 99%, but less than 100% of the alpha-amylase activity of one or more of the polypeptides of SEQ ID NOs: 1-14.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

Wash performance: In the present context the term “wash performance” is used as an enzyme's ability to remove starch or starch-containing stains present on the object to be cleaned during e.g. laundry or hard surface cleaning, such as dish wash. The term “wash performance” includes cleaning in general e.g. hard surface cleaning as in dish wash, but also wash performance on textiles such as laundry, and also industrial and institutional cleaning. The wash performance may be quantified by calculating the so-called Intensity value.

Wild-Type Enzyme: The term “wild-type” alpha-amylase means an alpha-amylase expressed by a naturally occurring microorganism, such as a bacterium, yeast, or filamentous fungus found in nature. The terms “wild-type enzyme” and “parent enzyme” can be used interchangeably when the parent enzyme is not a variant enzyme.

### Nomenclature

For purposes of the present invention, the nomenclature [Y/F] means that the amino acid at this position may be a tyrosine (Try, Y) or a phenylalanine (Phe, F). Likewise, the nomenclature [V/G/A/l] means that the amino acid at this position may be a valine (Val, V), glycine (Gly, G), alanine (Ala, A) or isoleucine (lie, 1), and so forth for other combinations as described herein. The amino acid X is defined such that it may be any of the 20 natural amino acids, unless otherwise stated.

### Conventions for Designation of Variants

For purposes of the present invention, the polypeptide disclosed in SEQ ID NO: 1 is used to determine the corresponding amino acid residue in another alpha-amylase. Alternatively, the polypeptide disclosed in SEQ ID NO: 2 or SEQ ID NO: 3 may be used to determine the corresponding amino acid residue in another alpha-amylase. The amino acid sequence of another alpha-amylase is aligned with the polypeptide disclosed in SEQ ID NO: 1, and based on the alignment, the amino acid position number corresponding to any amino acid residue in the polypeptide disclosed in SEQ ID NO: 2 or SEQ ID NO: 3 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later.

Identification of the corresponding amino acid residue in another alpha-amylase can be determined by an alignment of multiple polypeptide sequences using several computer programs including, but not limited to, MUSCLE (multiple sequence comparison by log-expectation; version 3.5 or later; Edgar, 2004, Nucleic Acids Research 32: 1792-1797), MAFFT (version 6.857 or later; Katoh and Kuma, 2002, Nucleic Acids Research 30: 3059-3066; Katoh et al., 2005, Nucleic Acids Research 33: 511-518; Katoh and Toh, 2007, Bioinformatics 23: 372-374; Katoh et al., 2009, Methods in Molecular Biology 537: 39-64; Katoh and Toh, 2010, Bioinformatics 26: 1899-1900), and EMBOSS EMMA employing ClustalW (1.83 or later; Thompson et al., 1994, Nucleic Acids Research 22: 4673-4680), using their respective default parameters.

When the other enzyme has diverged from the polypeptide of SEQ ID NO: 1 such that traditional sequence-based comparison fails to detect their relationship (Lindahl and Elofsson, 2000, J. Mol. Biol. 295: 613-615), other pairwise sequence comparison algorithms can be used. Greater sensitivity in sequence-based searching can be attained using search programs that utilize probabilistic representations of polypeptide families (profiles) to search databases. For example, the PSI-BLAST program generates profiles through an iterative database search process and is capable of detecting remote homologs (Atschul et al., 1997, Nucleic Acids Res. 25: 3389-3402).

Even greater sensitivity can be achieved if the family or superfamily for the polypeptide has one or more representatives in the protein structure databases. Programs such as GenTHREADER (Jones, 1999, J. Mol. Biol. 287: 797-815; McGuffin and Jones, 2003, Bioinformatics 19: 874-881) utilize information from a variety of sources (PSI-BLAST, secondary structure prediction, structural alignment profiles, and solvation potentials) as input to a neural network that predicts the structural fold for a query sequence. Similarly, the method of Gough et al., 2000, J. Mol. Biol. 313: 903-919, can be used to align a sequence of unknown structure with the superfamily models present in the SCOP database. These alignments can in turn be used to generate homology models for the polypeptide, and such models can be assessed for accuracy using a variety of tools developed for that purpose.

For proteins of known structure, several tools and resources are available for retrieving and generating structural alignments. For example, the SCOP superfamilies of proteins have been structurally aligned, and those alignments are accessible and downloadable. Two or more protein structures can be aligned using a variety of algorithms such as the distance alignment matrix (Holm and Sander, 1998, Proteins 33: 88-96) or combinatorial extension (Shindyalov and Bourne, 1998, Protein Engineering 11: 739-747), and implementation of these algorithms can additionally be utilized to query structure databases with a structure of interest in order to discover possible structural homologs (e.g., Holm and Park, 2000, Bioinformatics 16: 566-567).

In describing the variants, the nomenclature described below is adapted for ease of reference. The accepted IUPAC single letter or three letter amino acid abbreviation is employed.

Substitutions: For an amino acid substitution, the following nomenclature is used: Original amino acid, position, substituted amino acid. Accordingly, the substitution of threonine at position 226 with alanine is designated as “Thr226Ala” or “T226A”. In situations where the amino acid at a given position may be substituted for any other amino acid it is designated T226ACDEFGHIKLMNPQRSWVY. Accordingly, this means that threonine at position 226 may be substituted with one amino acid selected from the group of A, C, D, E, F, G, H, I, K, L, M, N, P, Q, R, S, W, V or Y. Likewise, in situations where the amino acid at a given position may be substituted for one amino acid selected from a specific group of amino acids, e.g. where the threonine at position 226 may be substituted with any of tyrosine, phenylalanine or histidine it is designated T226YFH. The different alterations at a given position may also be separated by a comma, e.g., “Arg170Tyr,Glu” or “R170Y,E” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”.

Multiple mutations are separated by addition marks (“+”), e.g., “Gly205Arg+Ser411 Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly, the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G-K-A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

### DETAILED DESCRIPTION OF THE INVENTION

### Parent Alpha-Amylases

The present invention relates to a polypeptide having amylase activity, wherein the polypeptide is a fusion (hybrid) polypeptide.

The present invention provides a polypeptide having alpha-amylase activity comprising an A-domain with at least 60% sequence identity with the A-domain of any of SEQ ID NOs: 1, 2, 3, 4 or 5, a B-domain with at least 60% sequence identity with the B-domain of any of SEQ ID NOs: 1, 2, 10, 11 or 12, and a C-domain with at least 60% sequence identity with the C-domain of any of SEQ ID NOs: 1, 2, 3, 4 or 5.

The present invention comprises a hybrid alpha-amylase of a parent polypeptide having alpha-amylase activity comprising an A domain, a B domain and a C domain, wherein the said hybrid is a fusion polypeptide.

In an embodiment, the A-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the A-domain of SEQ ID NO: 1.

In an embodiment, the A-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the A-domain of SEQ ID NO: 2.

In an embodiment, the A-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the A-domain of SEQ ID NO: 3.

In an embodiment, the A-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the A-domain of SEQ ID NO: 4.

In an embodiment, the A-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the A-domain of SEQ ID NO: 5.

In an embodiment, the B-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the B-domain of SEQ ID NO: 1.

In an embodiment, the B-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the B-domain of SEQ ID NO: 2.

In an embodiment, the B-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the B-domain of SEQ ID NO: 10.

In an embodiment, the B-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the B-domain of SEQ ID NO: 11.

In an embodiment, the B-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the B-domain of SEQ ID NO: 12.

In an embodiment, the C-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the C-domain of SEQ ID NO: 1.

In an embodiment, the C-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the C-domain of SEQ ID NO: 2.

In an embodiment, the C-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the C-domain of SEQ ID NO: 3.

In an embodiment, the C-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the C-domain of SEQ ID NO: 4.

In an embodiment, the C-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the C-domain of SEQ ID NO: 5.

The alpha-amylases may be produced by substituting the B-domain or a portion thereof of one alpha-amylase with the B-domain or a portion thereof of another alpha-amylase. The alpha-amylases also may be produced by substituting the A- and C-domains or a portion thereof of one alpha-amylase with the A- and C-domains or a portion thereof of another alpha-amylase.

For the Bacillus alpha-amylase (SEQ ID NO: 4) where the B-domain has been determined as amino acid residues 109-206, the sequence to be replaced by the corresponding sequence of another alpha-amylase starts at a position in the range of positions 93-113 and ending at a position in the range of positions 195-215, e.g., starting at a position in the range of positions 97-109 and ending at a position in the range of positions 199-215 or starting at a position in the range of positions 100-106 and ending at a position in the range of positions 202-208, in particular positions 109-206 of SEQ ID NO: 12. The A and C-domains of the Bacillus alpha-amylase were determined to be amino acid residues 1-108 (A1)+207-396 (A2) and 397-485, respectively. The alpha-amylases of the present invention may comprise an A1-domain starting at a position in the range of positions 1-5 and ending a position in the range of positions 94-114, e.g., starting at a position in the range of positions 1-3 and ending at a position in the range of positions 99-110 or starting at a position in the range of positions 1-3 and ending at a position in the range of positions 104-109, in particular positions 1-108 of SEQ ID NO: 4. The alpha-amylases of the present invention may comprise A2 and C-domains starting at a position in the range of positions 201-221 and ending at a position in the range of positions 478-514, e.g., starting at a position in the range of positions 203-211 and ending at a position in the range of positions 480-510 or starting at a position in the range of positions 205-216 and ending at a position in the range of positions 482-484 in particular positions 207-485 of SEQ ID NO: 4.

In one embodiment, the hybrid polypeptide comprises or consists of the amino acid sequence set forth in SEQ ID NO: 1 or 2. In another embodiment, the hybrid polypeptide is an allelic variant of the polypeptide of SEQ ID NO: 1 or 2.

In another embodiment, the parent polypeptide may comprise or consist of the amino acid sequence set forth in SEQ ID NO: 1. As stated elsewhere herein, the parent polypeptide may be any polypeptide having alpha-amylase activity and having at least 60% sequence identity to any one of the amino acid sequences as set forth in SEQ ID NOs: 1-14.

In one embodiment, the parent polypeptide has a sequence identity to the amino acid sequence set forth in SEQ ID NO: 2 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% and which parent polypeptide has alpha-amylase activity. In one embodiment, the amino acid sequence of the parent polypeptide differs by up to 10 amino acids, e.g., 1, 2, 3, 4, 5, 6, 7, 8, 9, or 10, from the amino acid sequences set forth in SEQ ID NOs: 1-14.

According to the present invention, a value of 1.0 corresponds to the performance observed for the parent polypeptide. A value above 1.0 indicates an improvement of performance of the hybrid tested compared to the parent polypeptide. Accordingly, any value of >1.0 is indicative for improvement of property, such as increased specific activity, of the hybrid polypeptide compared to the parent polypeptide.

In one aspect, the hybrid polypeptide is a parent polypeptide.

The parent alpha-amylase may be a polypeptide with at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity with any one of the polypeptides of SEQ ID Nos: 1-14.

In one aspect, the parent has a sequence identity to the polypeptide of SEQ ID NO: 1 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 1.

The parent preferably comprises or consists of the amino acid sequence of SEQ ID NO: 1. In one embodiment the parent comprises or consists of the polypeptide of SEQ ID NO: 1. In another embodiment, the parent is an allelic variant of the polypeptide of SEQ ID NO: 1.

In one aspect, the parent has a sequence identity to the polypeptide of SEQ ID NO: 2 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 2.

The parent preferably comprises or consists of the amino acid sequence of SEQ ID NO: 2.

In one embodiment the parent comprises or consists of the polypeptide of SEQ ID NO: 2. In another embodiment, the parent is an allelic variant of the polypeptide of SEQ ID NO: 2.

In one aspect, the parent has a sequence identity to the polypeptide of SEQ ID NO: 3 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 3.

The parent preferably comprises or consists of the amino acid sequence of SEQ ID NO: 3. In one embodiment the parent comprises or consists of the polypeptide of SEQ ID NO: 3. In another embodiment, the parent is an allelic variant of the polypeptide of SEQ ID NO: 3.

In one aspect, the parent has a sequence identity to the polypeptide of SEQ ID NO: 4 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 4.

The parent preferably comprises or consists of the amino acid sequence of SEQ ID NO: 4. In one embodiment the parent comprises or consists of the polypeptide of SEQ ID NO: 4. In another embodiment, the parent is an allelic variant of the polypeptide of SEQ ID NO: 4.

In one aspect, the parent has a sequence identity to the polypeptide of SEQ ID NO: 5 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 5.

The parent preferably comprises or consists of the amino acid sequence of SEQ ID NO: 5. In one embodiment the parent comprises or consists of the polypeptide of SEQ ID NO: 5. In another embodiment, the parent is an allelic variant of the polypeptide of SEQ ID NO: 5.

In one aspect, the parent has a sequence identity to the polypeptide of SEQ ID NO: 6 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 6.

The parent preferably comprises or consists of the amino acid sequence of SEQ ID NO: 6. In one embodiment the parent comprises or consists of the polypeptide of SEQ ID NO: 6. In another embodiment, the parent is an allelic variant of the polypeptide of SEQ ID NO: 6.

In one aspect, the parent has a sequence identity to the polypeptide of SEQ ID NO: 7 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 7.

The parent preferably comprises or consists of the amino acid sequence of SEQ ID NO: 7. In one embodiment the parent comprises or consists of the polypeptide of SEQ ID NO: 7. In another embodiment, the parent is an allelic variant of the polypeptide of SEQ ID NO: 7.

In one aspect, the parent has a sequence identity to the polypeptide of SEQ ID NO: 8 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 8.

The parent preferably comprises or consists of the amino acid sequence of SEQ ID NO: 8. In one embodiment the parent comprises or consists of the polypeptide of SEQ ID NO: 8. In another embodiment, the parent is an allelic variant of the polypeptide of SEQ ID NO: 8.

In one aspect, the parent has a sequence identity to the polypeptide of SEQ ID NO: 9 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 9.

The parent preferably comprises or consists of the amino acid sequence of SEQ ID NO: 9. In one embodiment the parent comprises or consists of the polypeptide of SEQ ID NO: 9. In another embodiment, the parent is an allelic variant of the polypeptide of SEQ ID NO: 9.

In one aspect, the parent has a sequence identity to the polypeptide of SEQ ID NO: 10 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 10.

The parent preferably comprises or consists of the amino acid sequence of SEQ ID NO: 10. In one embodiment the parent comprises or consists of the polypeptide of SEQ ID NO: 10. In another embodiment, the parent is an allelic variant of the polypeptide of SEQ ID NO: 10.

In one aspect, the parent has a sequence identity to the polypeptide of SEQ ID NO: 11 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 11.

The parent preferably comprises or consists of the amino acid sequence of SEQ ID NO: 11. In one embodiment the parent comprises or consists of the polypeptide of SEQ ID NO: 11. In another embodiment, the parent is an allelic variant of the polypeptide of SEQ ID NO: 11.

In one aspect, the parent has a sequence identity to the polypeptide of SEQ ID NO: 12 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 12.

The parent preferably comprises or consists of the amino acid sequence of SEQ ID NO: 12. In one embodiment the parent comprises or consists of the polypeptide of SEQ ID NO: 12. In another embodiment, the parent is an allelic variant of the polypeptide of SEQ ID NO: 12.

In one aspect, the parent has a sequence identity to the polypeptide of SEQ ID NO: 13 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 13.

The parent preferably comprises or consists of the amino acid sequence of SEQ ID NO: 13. In one embodiment the parent comprises or consists of the polypeptide of SEQ ID NO: 13. In another embodiment, the parent is an allelic variant of the polypeptide of SEQ ID NO: 13.

In one aspect, the parent has a sequence identity to the polypeptide of SEQ ID NO: 14 of at least 60% e.g., at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, or 100% which have alpha-amylase activity. In one embodiment, the amino acid sequence of the parent differs by no more than ten amino acids, e.g., by five amino acids, by four amino acids, by three amino acids, by two amino acids, and by one amino acid from the polypeptide of SEQ ID NO: 14.

The parent preferably comprises or consists of the amino acid sequence of SEQ ID NO: 14. In one embodiment the parent comprises or consists of the polypeptide of SEQ ID NO: 14. In another embodiment, the parent is an allelic variant of the polypeptide of SEQ ID NO: 14.

The amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 or a fragment thereof, may be used to design nucleic acid probes to identify and clone DNA encoding a parent from strains of different genera or species according to methods well known in the art. In particular, such probes can be used for hybridization with the genomic or cDNA of the genus or species of interest, following standard Southern blotting procedures, in order to identify and isolate the corresponding gene therein. Such probes can be considerably shorter than the entire sequence, but should be at least 14, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a parent. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with parent or a subsequence thereof, the carrier material is used in a Southern blot.

For purposes of the present invention, hybridization indicates that the polynucleotide hybridizes to a labeled nucleotide probe corresponding to a polynucleotide encoding SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or a subsequence thereof, under low to very high stringency conditions. Molecules to which the probe hybridizes can be detected using, for example, X-ray film or any other detection means known in the art.

In one aspect, the nucleic acid probe is a polynucleotide that encodes the polypeptide of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or a fragment thereof.

For long probes of at least 100 nucleotides in length, very low to very high stringency conditions are defined as prehybridization and hybridization at 42° C. in 5×SSPE, 0.3% SDS, 200 micrograms/ml sheared and denatured salmon sperm DNA, and either 25% formamide for very low and low stringencies, 35% formamide for medium and medium-high stringencies, or 50% formamide for high and very high stringencies, following standard Southern blotting procedures for 12 to 24 hours optimally. The carrier material is finally washed three times each for 15 minutes using 2×SSC, 0.2% SDS at 45° C. (very low stringency), 50° C. (low stringency), 55° C. (medium stringency), 60° C. (medium-high stringency), 65° C. (high stringency), or 70° C. (very high stringency).

For short probes that are about 15 nucleotides to about 70 nucleotides in length, stringency conditions are defined as prehybridization and hybridization at about 5° C. to about 10° C. below the calculated Tₘ using the calculation according to Bolton and McCarthy (1962, Proc. Natl. Acad. Sci. USA 48: 1390) in 0.9 M NaCl, 0.09 M Tris-HCl pH 7.6, 6 mM EDTA, 0.5% NP-40, 1×Denhardt's solution, 1 mM sodium pyrophosphate, 1 mM sodium monobasic phosphate, 0.1 mM ATP, and 0.2 mg of yeast RNA per ml following standard Southern blotting procedures for 12 to 24 hours optimally. The carrier material is finally washed once in 6×SCC plus 0.1% SDS for 15 minutes and twice each for 15 minutes using 6×SSC at 5° C. to 10° C. below the calculated Tₘ.

The parent may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

The parent may be a bacterial alpha-amylase. For example, the parent may be a gram-positive bacterial polypeptide such as a Bacillus, Clostridium, Enterococcus, Geobacillus, Lactobacillus, Lactococcus, Oceanobacillus, Staphylococcus, Streptococcus, or Streptomyces alpha-amylase, or a gram-negative bacterial polypeptide such as a Campylobacter, E. coli, Flavobacterium, Fusobacterium, Helicobacter, Ilyobacter, Neisseria, Pseudomonas, Salmonella, or Ureaplasma alpha-amylase.

In one aspect, the parent is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis alpha-amylase.

In another aspect, the parent is a Streptococcus equisimilis, Streptococcus pyogenes, Streptococcus uberis, or Streptococcus equi subsp. Zooepidemicus alpha-amylase.

In another aspect, the parent is a Streptomyces achromogenes, Streptomyces avermitilis, Streptomyces coelicolor, Streptomyces griseus, or Streptomyces lividans alpha-amylase.

In another aspect, the parent is a Bacillus sp. alpha-amylase, e.g., the alpha-amylase of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13 or SEQ ID NO: 14.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

The parent may be identified and obtained from other sources including microorganisms isolated from nature (e.g., soil, composts, water, etc.) or DNA samples obtained directly from natural materials (e.g., soil, composts, water, etc,) using the above-mentioned probes. Techniques for isolating microorganisms and DNA directly from natural habitats are well known in the art. The polynucleotide encoding a parent may then be derived by similarly screening a genomic or cDNA library of another microorganism or mixed DNA sample. Once a polynucleotide encoding a parent has been detected with a probe(s), the polynucleotide may be isolated or cloned by utilizing techniques that are known to those of ordinary skill in the art (see, e.g., Sambrook et al., 1989, supra).

The parent may be a hybrid polypeptide in which a portion of one polypeptide is fused at the N-terminus or the C-terminus of a portion of another polypeptide.

The parent may also be a fused polypeptide or cleavable fusion polypeptide in which one polypeptide is fused at the N-terminus or the C-terminus of another polypeptide. A fused polypeptide is produced by fusing a polynucleotide encoding one polypeptide to a polynucleotide encoding another polypeptide. Techniques for producing fusion polypeptides are known in the art, and include ligating the coding sequences encoding the polypeptides so that they are in frame and that expression of the fused polypeptide is under control of the same promoter(s) and terminator. Fusion proteins may also be constructed using intein technology in which fusions are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Preparation of Variants

The present invention relates to a method for obtaining a variant having alpha-amylase activity, comprising (a) introducing into a parent alpha-amylase a modification at one or more positions corresponding to positions 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering, wherein each modification is independently a substitution or deletion, and said variant has alpha-amylase activity; and (b) recovering said variant.

The method for obtaining a variant having alpha-amylase activity, comprising (a) introducing into a parent alpha-amylase a modification at one or more positions corresponding to positions 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476 of the amino acid sequence as set forth in SEQ ID NO: 1, wherein each modification is independently a substitution or deletion, and said variant has alpha-amylase activity; and (b) recovering said variant.

In one aspect, the method for obtaining a variant having alpha-amylase activity, comprising (a) introducing into a parent alpha-amylase a modification at one or more positions corresponding to positions 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476 of the amino acid sequence as set forth in SEQ ID NOs: 2, 3, 4, 5, 6, 7, 8, 9, 10, 11, 12, 13 or 14, using SEQ ID NO: 1 for numbering, and wherein each modification is independently a substitution or deletion, and said variant has alpha-amylase activity; and (b) recovering said variant.

In one embodiment, the modification is a substitution. In one embodiment, the modification is a deletion.

In another embodiment, the method for obtaining a variant having alpha-amylase activity, comprising (a) introducing into a parent alpha-amylase a modification at one or more positions, wherein the modification is selected from H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A of the amino acid sequence as set forth in SEQ ID NO: 1, and (b) recovering the variant.

In another embodiment, the method for obtaining a variant having alpha-amylase activity, comprising (a) introducing into a parent alpha-amylase a substitution at one or more positions, wherein the substitution is selected from N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180Q, R180S, S181L, S181T, T182D, T182E, T182G, T182I, T182L, T182M, T182S, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A of the amino acid sequence as set forth in SEQ ID NOs: 2, 3, 4, 5, 6, 7, 8, 9, 10, 11, 12, 13 or 14, using SEQ ID NO: 1 for numbering and (b) recovering the variant.

In one embodiment, the method further comprises introducing to the parent alpha-amylase a deletion in one or more positions, wherein the deletion is selected from: H1*, H2*, R180*, S181*, T182* and G183* of the polypeptide of SEQ ID Nos: 1, 2, 3, 4, 5, 6, 7, 8, 9, 10, 11, 12, 13 or 14, using SEQ ID NO: 1 for numbering, and recovering the variant.

The variants may be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more (several) mutations are created at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests at the plasmid and the oligonucleotide is the same, permitting sticky ends of the plasmid and insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., U.S. Patent Application Publication No. 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al. (2004, Nature 432: 1050-1054) and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Alpha-Amylase Variants

The present invention relates to alpha-amylase variants of a parent alpha-amylase comprising a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity.

In one aspect, the alpha-amylase variants comprise a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity.

In one aspect, the alpha-amylase variants comprise a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variants comprise a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the alpha-amylase variant has an improved property relative to said parent polypeptide.

In one aspect, alpha-amylase variants comprising a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering, and the variant has alpha-amylase activity and wherein the alpha-amylase variant has an improved property relative to said parent polypeptide.

In one aspect, alpha-amylase variants comprising a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering, and the variant has alpha-amylase activity and wherein the alpha-amylase variant has an improved property relative to said parent polypeptide and wherein said parent polypeptide is of SEQ ID NO: 1, 2 or 3.

In another embodiment, the variant at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the parent polypeptide.

In another embodiment, the variant at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the parent polypeptide having alpha-amylase activity.

In an embodiment, the variant has sequence identity of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% to the hybrid polypeptide.

In an embodiment, the variant has sequence identity of at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100% to the hybrid polypeptide having alpha-amylase activity.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 1.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 2.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 3.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 4.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 5.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 6.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 7.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 8.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 9.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 10.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 11.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 12.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 13.

In another embodiment, the variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the polypeptide of SEQ ID NO: 14.

In one aspect, the substituted amino acid residue is different from the naturally-occurring amino acid residue in that position. In one embodiment, the substitution is selected from the group consisting of A, C, D, E, F, G, H, I, K, L, M, N, P, Q, R, S, T, V, W and Y, with the proviso that the substituted amino acid residue is different from the naturally-occurring amino acid residue in that position.

In one aspect, the number of modifications is 1-50, e.g., 1-45, 1-40, 1-35, 1-30, 1-25, 1-20, 1-15, 1-10 or 1-5, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 modifications.

In one aspect, the number of substitutions is 1-50, e.g., 1-45, 1-40, 1-35, 1-30, 1-25, 1-20, 1-15, 1-10 or 1-5, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 substitutions.

In one aspect, the number of deletions is 1-50, e.g., 1-45, 1-40, 1-35, 1-30, 1-25, 1-20, 1-15, 1-10 or 1-5, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 deletions.

The above-mentioned variants having substitutions should be understood as encompassing all possible combinations of one or more substitutions at the specified positions.

In one aspect, the alpha-amylase variants comprise a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In one aspect, the alpha-amylase variants comprising a modification at one or more (e.g., several) positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at two positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at three positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at four positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at five positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at six positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at seven positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at eight positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at nine positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at ten positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at eleven positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at twelve positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at thirteen positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at fourteen positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at fifteen positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at sixteen positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at seventeen positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at eighteen positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at nineteen positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at twenty positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at twenty one positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at twenty two positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at twenty three positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at twenty four positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at twenty five positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at twenty six positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at twenty seven positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at each positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, the variant comprises or consists of a deletion at a position corresponding to position 1. In another aspect, the amino acid at a position corresponding to position 1 is deleted of the polypeptide of SEQ ID NO: 1. In another aspect, the variant comprises or consists of deletion H1* of the polypeptide of SEQ ID NO: 1 In another aspect, the variant comprises or consists of a deletion at a position corresponding to position 2. In another aspect, the amino acid at a position corresponding to position 2 is deleted of the polypeptide of SEQ ID NO: 1. In another aspect, the variant comprises or consists of deletion H2* of the polypeptide of SEQ ID NO: 1.

In another aspect, the variant comprises or consists of a substitution at a position corresponding to position 3 of SEQ ID NO: 1. In another aspect, the amino acid at a position corresponding to position 3 is substituted with Ala, Arg, Asn, Asp, Cys, Gln, Glu, His, lie, Leu, Lys, Met, Phe, Pro, Ser, Thr, Trp, Tyr, or Val. In another aspect, the variant comprises or consists of the substitution N3S or N3D or N3G of the polypeptide of SEQ ID NO: 1.

In another aspect, the variant comprises or consists of a substitution at a position corresponding to position 7 of SEQ ID NO: 1. In another aspect, the amino acid at a position corresponding to position 7 is substituted with Ala, Arg, Asn, Asp, Cys, Gln, Glu, His, lie, Leu, Lys, Met, Phe, Pro, Ser, Thr, Trp, Tyr, or Val. In another aspect, the variant comprises or consists of the substitution G7A or G7H or G7S of the polypeptide of SEQ ID NO: 1.

In another aspect, the variant comprises or consists of a substitution at a position corresponding to position 26 of SEQ ID NO: 1. In another aspect, the amino acid at a position corresponding to position 26 is substituted with Ala, Asn, Asp, Cys, Gln, Glu, Gly, His, lie, Leu, Lys, Met, Phe, Pro, Ser, Thr, Trp, Tyr, or Val. In another aspect, the variant comprises or consists of the substitution R26Y of the polypeptide of SEQ ID NO: 1.

In another aspect, the variant comprises or consists of a substitution at a position corresponding to position 31 of SEQ ID NO: 1. In another aspect, the amino acid at a position corresponding to position 31 is substituted with Arg, Asn, Asp, Cys, Gln, Glu, Gly, His, lie, Leu, Lys, Met, Phe, Pro, Ser, Thr, Trp, Tyr, or Val. In another aspect, the variant comprises or consists of the substitution A31S of the polypeptide of SEQ ID NO: 1.

In another aspect, the variant comprises or consists of a substitution at a position corresponding to position 37 of SEQ ID NO: 1. In another aspect, the amino acid at a position corresponding to position 37 is substituted with Ala, Asn, Asp, Cys, Gln, Glu, Gly, His, lie, Leu, Lys, Met, Phe, Pro, Ser, Thr, Trp, Tyr, or Val. In another aspect, the variant comprises or consists of the substitution R37A or R37N or R37V of the polypeptide of SEQ ID NO: 1.

In another aspect, the variant comprises or consists of a substitution at a position corresponding to position 40 of SEQ ID NO: 1. In another aspect, the amino acid at a position corresponding to position 40 is substituted with Ala, Arg, Asn, Asp, Cys, Gln, Glu, Gly, His, lie, Leu, Lys, Met, Phe, Pro, Ser, Trp, Tyr, or Val. In another aspect, the variant comprises or consists of the substitution T40N or T40G of the polypeptide of SEQ ID NO: 1.

In another aspect, the variant comprises or consists of a substitution at a position corresponding to position 41 of SEQ ID NO: 1. In another aspect, the amino acid at a position corresponding to position 41 is substituted with Arg, Asn, Asp, Cys, Gln, Glu, Gly, His, lie, Leu, Lys, Met, Phe, Pro, Ser, Trp, Thr, Tyr, or Val. In another aspect, the variant comprises or consists of the substitution A41D of the polypeptide of SEQ ID NO: 1.

In another aspect, the variant comprises or consists of a substitution at a position corresponding to position 54 of SEQ ID NO: 1. In another aspect, the amino acid at a position corresponding to position 54 is substituted with Ala, Arg, Asp, Cys, Gln, Glu, Gly, His, lie, Leu, Lys, Met, Phe, Pro, Ser, Thr, Trp, Tyr, or Val. In another aspect, the variant comprises or consists of the substitution N54S of the polypeptide of SEQ ID NO: 1.

In another aspect, the variant comprises or consists of a substitution at a position corresponding to position 56 of SEQ ID NO: 1. In another aspect, the amino acid at a position corresponding to position 56 is substituted with Ala, Arg, Asn, Asp, Cys, Gln, Glu, Gly, His, lie, Leu, Lys, Met, Phe, Pro, Ser, Thr, Trp, or Tyr. In another aspect, the variant comprises or consists of the substitution V56T of the polypeptide of SEQ ID NO: 1.

In another aspect, the variant comprises or consists of a substitution at a position corresponding to position 60 of SEQ ID NO: 1. In another aspect, the amino acid at a position corresponding to position 60 is substituted with Arg, Asn, Asp, Cys, Gln, Glu, Gly, His, lie, Leu, Lys, Met, Phe, Pro, Ser, Thr, Trp, Tyr, or Val. In another aspect, the variant comprises or consists of the substitution A60P or A60V of the polypeptide of SEQ ID NO: 1.

In another aspect, the variant comprises or consists of a substitution at a position corresponding to position 63 of SEQ ID NO: 1. In another aspect, the amino acid at a position corresponding to position 63 is substituted with Ala, Arg, Asn, Asp, Cys, Gln, Glu, Gly, His, lie, Lys, Met, Phe, Pro, Ser, Thr, Trp, Tyr, or Val. In another aspect, the variant comprises or consists of the substitution L63F of the polypeptide of SEQ ID NO: 1.

In another aspect, the variant comprises or consists of a substitution at a position corresponding to position 72 of SEQ ID NO: 1. In another aspect, the amino acid at a position corresponding to position 72 is substituted with Ala, Arg, Asn, Asp, Cys, Gln, Glu, Gly, His, lie, Leu, Met, Phe, Pro, Ser, Thr, Trp, Tyr, or Val. In another aspect, the variant comprises or consists of the substitution K72R of the polypeptide of SEQ ID NO: 1.

In another aspect, the variant comprises or consists of a substitution at a position corresponding to position 73 of SEQ ID NO: 1. In another aspect, the amino acid at a position corresponding to position 73 is substituted with Ala, Arg, Asn, Asp, Cys, Gln, Glu, His, lie, Leu, Lys, Met, Phe, Pro, Ser, Thr, Trp, Tyr, or Val. In another aspect, the variant comprises or consists of the substitution G73L of the polypeptide of SEQ ID NO: 1.

In another aspect, the variant comprises or consists of a substitution at a position corresponding to position 93 of SEQ ID NO: 1. In another aspect, the amino acid at a position corresponding to position 93 is substituted with Ala, Arg, Asn, Asp, Cys, Gln, Glu, Gly, His, lie, Leu, Met, Phe, Pro, Ser, Thr, Trp, Tyr, or Val. In another aspect, the variant comprises or consists of the substitution K93D of the polypeptide of SEQ ID NO: 1.

In another aspect, the variant comprises or consists of a substitution at a position corresponding to position 94 of SEQ ID NO: 1. In another aspect, the amino acid at a position corresponding to position 94 is substituted with Ala, Arg, Asp, Cys, Gln, Glu, Gly, His, lie, Leu, Lys, Met, Phe, Pro, Ser, Thr, Trp, Tyr, or Val. In another aspect, the variant comprises or consists of the substitution N94D of the polypeptide of SEQ ID NO: 1.

In another aspect, the variant comprises or consists of a substitution at a position corresponding to position 98 of SEQ ID NO: 1. In another aspect, the amino acid at a position corresponding to position 98 is substituted with Ala, Arg, Asn, Asp, Cys, Glu, Gly, His, lie, Leu, Lys, Met, Phe, Pro, Ser, Thr, Trp, Tyr, or Val. In another aspect, the variant comprises or consists of the substitution Q98L of the polypeptide of SEQ ID NO: 1.

In another aspect, the variant comprises or consists of a substitution at a position corresponding to position 103 of SEQ ID NO: 1. In another aspect, the amino acid at a position corresponding to position 103 is substituted with Ala, Arg, Asn, Asp, Cys, Gln, Glu, Gly, His, lie, Leu, Lys, Met, Phe, Pro, Ser, Thr, Trp, or Tyr. In another aspect, the variant comprises or consists of the substitution V103 or VA103I of the polypeptide of SEQ ID NO: 1.

In another aspect, the variant comprises or consists of a substitution at a position corresponding to position 104 of SEQ ID NO: 1. In another aspect, the amino acid at a position corresponding to position 104 is substituted with Ala, Arg, Asn, Asp, Cys, Gln, Glu, Gly, His, lie, Leu, Lys, Met, Phe, Pro, Ser, Thr, Trp, or Tyr. In another aspect, the variant comprises or consists of the substitution V104M of the polypeptide of SEQ ID NO: 1.

In another aspect, the variant comprises or consists of a substitution at a position corresponding to position 105 of SEQ ID NO: 1. In another aspect, the amino acid at a position corresponding to position 105 is substituted with Ala, Arg, Asn, Asp, Cys, Gln, Glu, Gly, His, lie, Leu, Lys, Phe, Pro, Ser, Thr, Trp, Tyr or Val. In another aspect, the variant comprises or consists of the substitution M105I or M105L of the polypeptide of SEQ ID NO: 1.

In another aspect, the variant comprises or consists of a substitution at a position corresponding to position 106 of SEQ ID NO: 1. In another aspect, the amino acid at a position corresponding to position 106 is substituted with Ala, Arg, Asp, Cys, Gln, Glu, Gly, His, lie, Leu, Lys, Met, Phe, Pro, Ser, Thr, Trp, Tyr, or Val. In another aspect, the variant comprises or consists of the substitution N106D of the polypeptide of SEQ ID NO: 1.

In another aspect, the variant comprises or consists of a substitution at a position corresponding to position 109 of SEQ ID NO: 1. In another aspect, the amino acid at a position corresponding to position 109 is substituted with Arg, Asn, Asp, Cys, Gln, Glu, Gly, His, lie, Leu, Lys, Met, Phe, Pro, Ser, Thr, Trp, Tyr, or Val. In another aspect, the variant comprises or consists of the substitution A109G of the polypeptide of SEQ ID NO: 1.

In another aspect, the variant comprises or consists of a substitution at a position corresponding to position 110 of SEQ ID NO: 1. In another aspect, the amino acid at a position corresponding to position 110 is substituted with Ala, Arg, Asn, Asp, Cys, Gln, Glu, His, lie, Leu, Lys, Met, Phe, Pro, Ser, Thr, Trp, Tyr, or Val. In another aspect, the variant comprises or consists of the substitution G110K of the polypeptide of SEQ ID NO: 1.

In another aspect, the variant comprises or consists of a substitution at a position corresponding to position 111 of SEQ ID NO: 1. In another aspect, the amino acid at a position corresponding to position 111 is substituted with Arg, Asn, Asp, Cys, Gln, Glu, Gly, His, lie, Leu, Lys, Met, Phe, Pro, Ser, Thr, Trp, Tyr, or Val. In another aspect, the variant comprises or consists of the substitution A111G or A111 L of the polypeptide of SEQ ID NO: 1.

In another aspect, the variant comprises or consists of a substitution at a position corresponding to position 113 of SEQ ID NO: 1. In another aspect, the amino acid at a position corresponding to position 113 is substituted with Ala, Arg, Asn, Asp, Cys, Gln, Glu, His, lie, Leu, Lys, Met, Phe, Pro, Ser, Thr, Trp, Tyr, or Val. In another aspect, the variant comprises or consists of the substitution G113A or G113Q or G113S of the polypeptide of SEQ ID NO: 1.

In another aspect, the variant comprises or consists of a substitution at a position corresponding to position 114 of SEQ ID NO: 1. In another aspect, the amino acid at a position corresponding to position 114 is substituted with Ala, Arg, Asn, Asp, Cys, Gln, Glu, Gly, His, lie, Leu, Lys, Met, Phe, Pro, Ser, Trp, Tyr, or Val. In another aspect, the variant comprises or consists of the substitution T114K of the polypeptide of SEQ ID NO: 1.

In another aspect, the variant comprises or consists of a substitution at a position corresponding to position 116 of SEQ ID NO: 1. In another aspect, the amino acid at a position corresponding to position 116 is substituted with Ala, Arg, Asn, Asp, Cys, Gln, Glu, Gly, His, lie, Leu, Lys, Met, Pro, Ser, Thr, Trp, Tyr, or Val. In another aspect, the variant comprises or consists of the substitution F116L or F116N or F116Q or F116W of the polypeptide of SEQ ID NO: 1.

In another aspect, the variant comprises or consists of a substitution at a position corresponding to position 117 of SEQ ID NO: 1. In another aspect, the amino acid at a position corresponding to position 117 is substituted with Ala, Arg, Asn, Asp, Cys, Gln, Glu, Gly, His, lie, Leu, Lys, Met, Phe, Pro, Ser, Thr, Trp, or Tyr. In another aspect, the variant comprises or consists of the substitution V117G of the polypeptide of SEQ ID NO: 1.

In another aspect, the variant comprises or consists of a substitution at a position corresponding to position 118 of SEQ ID NO: 1. In another aspect, the amino acid at a position corresponding to position 118 is substituted with Ala, Arg, Asn, Cys, Gln, Glu, Gly, His, lie, Leu, Lys, Met, Phe, Pro, Ser, Thr, Trp, Tyr, or Val. In another aspect, the variant comprises or consists of the substitution D118T or D118Q of the polypeptide of SEQ ID NO: 1.

In another aspect, the variant comprises or consists of a substitution at a position corresponding to position 123 of SEQ ID NO: 1. In another aspect, the amino acid at a position corresponding to position 123 is substituted with Ala, Arg, Asn, Cys, Gln, Glu, Gly, His, lie, Leu, Lys, Met, Phe, Pro, Ser, Thr, Trp, Tyr, or Val. In another aspect, the variant comprises or consists of the substitution D123N of the polypeptide of SEQ ID NO: 1.

In another aspect, the variant comprises or consists of a substitution at a position corresponding to position 126 of SEQ ID NO: 1. In another aspect, the amino acid at a position corresponding to position 126 is substituted with Ala, Arg, Asp, Cys, Gln, Glu, Gly, His, lie, Leu, Lys, Met, Phe, Pro, Ser, Thr, Trp, Tyr, or Val. In another aspect, the variant comprises or consists of the substitution N126D of the polypeptide of SEQ ID NO: 1.

In another aspect, the variant comprises or consists of a substitution at a position corresponding to position 128 of SEQ ID NO: 1. In another aspect, the amino acid at a position corresponding to position 128 is substituted with Ala, Arg, Asp, Cys, Gln, Glu, Gly, His, lie, Leu, Lys, Met, Phe, Pro, Ser, Thr, Trp, Tyr, or Val. In another aspect, the variant comprises or consists of the substitution N128Y of the polypeptide of SEQ ID NO: 1.

In another aspect, the variant comprises or consists of a substitution at a position corresponding to position 131 of SEQ ID NO: 1. In another aspect, the amino acid at a position corresponding to position 131 is substituted with Ala, Arg, Asn, Asp, Cys, Gln, Glu, Gly, His, lie, Leu, Lys, Met, Phe, Pro, Ser, Trp, Tyr, or Val. In another aspect, the variant comprises or consists of the substitution T131I or T131V of the polypeptide of SEQ ID NO: 1.

In another aspect, the variant comprises or consists of a substitution at a position corresponding to position 132 of SEQ ID NO: 1. In another aspect, the amino acid at a position corresponding to position 132 is substituted with Ala, Arg, Asn, Asp, Cys, Gln, Glu, Gly, His, lie, Leu, Lys, Met, Phe, Pro, Thr, Trp, Tyr, or Val. In another aspect, the variant comprises or consists of the substitution S132D or S132L of the polypeptide of SEQ ID NO: 1.

In another aspect, the variant comprises or consists of a substitution at a position corresponding to position 134 of SEQ ID NO: 1. In another aspect, the amino acid at a position corresponding to position 134 is substituted with Ala, Arg, Asn, Asp, Cys, Gln, Glu, Gly, His, lie, Leu, Lys, Met, Phe, Pro, Ser, Trp, Tyr, or Val. In another aspect, the variant comprises or consists of the substitution T134D of the polypeptide of SEQ ID NO: 1.

In another aspect, the variant comprises or consists of a substitution at a position corresponding to position 135 of SEQ ID NO: 1. In another aspect, the amino acid at a position corresponding to position 135 is substituted with Ala, Arg, Asn, Asp, Cys, Gln, Glu, Gly, His, lie, Leu, Lys, Met, Phe, Pro, Ser, Thr, Trp, or Val. In another aspect, the variant comprises or consists of the substitution Y135E of the polypeptide of SEQ ID NO: 1.

In another aspect, the variant comprises or consists of a substitution at a position corresponding to position 136 of SEQ ID NO: 1. In another aspect, the amino acid at a position corresponding to position 136 is substituted with Ala, Arg, Asn, Asp, Cys, Glu, Gly, His, lie, Leu, Lys, Met, Phe, Pro, Ser, Thr, Trp, Tyr, or Val. In another aspect, the variant comprises or consists of the substitution Q136T or Q136K of the polypeptide of SEQ ID NO: 1.

In another aspect, the variant comprises or consists of a substitution at a position corresponding to position 137 of SEQ ID NO: 1. In another aspect, the amino acid at a position corresponding to position 137 is substituted with Ala, Arg, Asn, Asp, Cys, Gln, Glu, Gly, His, Leu, Lys, Met, Phe, Pro, Ser, Thr, Trp, Tyr, or Val. In another aspect, the variant comprises or consists of the substitution 1137F of the polypeptide of SEQ ID NO: 1.

In another aspect, the variant comprises or consists of a substitution at a position corresponding to position 138 of SEQ ID NO: 1. In another aspect, the amino acid at a position corresponding to position 138 is substituted with Ala, Arg, Asn, Asp, Cys, Glu, Gly, His, lie, Leu, Lys, Met, Phe, Pro, Ser, Thr, Trp, Tyr, or Val. In another aspect, the variant comprises or consists of the substitution Q138V of the polypeptide of SEQ ID NO: 1.

In another aspect, the variant comprises or consists of a substitution at a position corresponding to position 140 of SEQ ID NO: 1. In another aspect, the amino acid at a position corresponding to position 140 is substituted with Ala, Arg, Asn, Asp, Cys, Gln, Glu, Gly, His, lie, Leu, Lys, Met, Phe, Pro, Ser, Thr, Tyr, or Val. In another aspect, the variant comprises or consists of the substitution W140S or W140Y or W140M of the polypeptide of SEQ ID NO: 1.

In another aspect, the variant comprises or consists of a substitution at a position corresponding to position 142 of SEQ ID NO: 1. In another aspect, the amino acid at a position corresponding to position 142 is substituted with Ala, Arg, Asn, Asp, Cys, Gln, Glu, Gly, His, lie, Leu, Met, Phe, Pro, Ser, Thr, Trp, Tyr, or Val. In another aspect, the variant comprises or consists of the substitution K142E or K142G or K142P of the polypeptide of SEQ ID NO: 1.

In another aspect, the variant comprises or consists of a substitution at a position corresponding to position 144 of SEQ ID NO: 1. In another aspect, the amino acid at a position corresponding to position 144 is substituted with Ala, Arg, Asn, Cys, Gln, Glu, Gly, His, lie, Leu, Lys, Met, Phe, Pro, Ser, Thr, Trp, Tyr, or Val. In another aspect, the variant comprises or consists of the substitution D144N of the polypeptide of SEQ ID NO: 1.

In another aspect, the variant comprises or consists of a substitution at a position corresponding to position 146 of SEQ ID NO: 1. In another aspect, the amino acid at a position corresponding to position 146 is substituted with Ala, Arg, Asn, Asp, Cys, Gln, Glu, Gly, His, lie, Leu, Lys, Met, Phe, Ser, Thr, Trp, Tyr, or Val. In another aspect, the variant comprises or consists of the substitution P146H or P146S of the polypeptide of SEQ ID NO: 1.

In another aspect, the variant comprises or consists of a substitution at a position corresponding to position 151 of SEQ ID NO: 1. In another aspect, the amino acid at a position corresponding to position 151 is substituted with Ala, Arg, Asn, Asp, Cys, Gln, Glu, Gly, His, lie, Leu, Lys, Met, Phe, Pro, Ser, Trp, Tyr, or Val. In another aspect, the variant comprises or consists of the substitution T151A of the polypeptide of SEQ ID NO: 1.

In another aspect, the variant comprises or consists of a substitution at a position corresponding to position 152 of SEQ ID NO: 1. In another aspect, the amino acid at a position corresponding to position 152 is substituted with Ala, Arg, Asn, Asp, Cys, Gln, Glu, Gly, His, lie, Leu, Lys, Met, Phe, Pro, Ser, Thr, Trp, or Val. In another aspect, the variant comprises or consists of the substitution Y152L of the polypeptide of SEQ ID NO: 1.

In another aspect, the variant comprises or consists of a substitution at a position corresponding to position 155 of SEQ ID NO: 1. In another aspect, the amino acid at a position corresponding to position 155 is substituted with Ala, Arg, Asn, Asp, Cys, Gln, Glu, Gly, His, lie, Leu, Lys, Met, Pro, Ser, Thr, Trp, Tyr, or Val. In another aspect, the variant comprises or consists of the substitution F155W of the polypeptide of SEQ ID NO: 1.

In another aspect, the variant comprises or consists of a substitution at a position corresponding to position 159 of SEQ ID NO: 1. In another aspect, the amino acid at a position corresponding to position 159 is substituted with Ala, Arg, Asn, Asp, Cys, Gln, Glu, Gly, His, lie, Leu, Lys, Met, Phe, Pro, Ser, Thr, Tyr, or Val. In another aspect, the variant comprises or consists of the substitution W159H or W159L of the polypeptide of SEQ ID NO: 1.

In another aspect, the variant comprises or consists of a substitution at a position corresponding to position 160 of SEQ ID NO: 1. In another aspect, the amino acid at a position corresponding to position 160 is substituted with Ala, Arg, Asn, Asp, Cys, Gln, Glu, Gly, His, lie, Leu, Lys, Met, Phe, Pro, Ser, Thr, Trp, or Val. In another aspect, the variant comprises or consists of the substitution Y160P of the polypeptide of SEQ ID NO: 1.

In another aspect, the variant comprises or consists of a substitution at a position corresponding to position 165 of SEQ ID NO: 1. In another aspect, the amino acid at a position corresponding to position 165 is substituted with Ala, Arg, Asn, Asp, Cys, Gln, Glu, Gly, His, lie, Leu, Lys, Met, Phe, Pro, Ser, Trp, Tyr, or Val. In another aspect, the variant comprises or consists of the substitution T165V of the polypeptide of SEQ ID NO: 1.

In another aspect, the variant comprises or consists of a substitution at a position corresponding to position 167 of SEQ ID NO: 1. In another aspect, the amino acid at a position corresponding to position 167 is substituted with Ala, Arg, Asn, Asp, Cys, Gln, Glu, Gly, His, lie, Leu, Lys, Met, Phe, Pro, Ser, Thr, Tyr, or Val. In another aspect, the variant comprises or consists of the substitution W167A or W167D or W167E or W167F or W167P or W167S of the polypeptide of SEQ ID NO: 1.

In another aspect, the variant comprises or consists of a substitution at a position corresponding to position 169 of SEQ ID NO: 1. In another aspect, the amino acid at a position corresponding to position 169 is substituted with Ala, Arg, Asn, Asp, Cys, Gln, Gly, Gly, His, lie, Leu, Lys, Met, Phe, Pro, Ser, Thr, Trp, Tyr, or Val. In another aspect, the variant comprises or consists of the substitution E169V or E169I or E169Q of the polypeptide of SEQ ID NO: 1.

In another aspect, the variant comprises or consists of a substitution at a position corresponding to position 172 of SEQ ID NO: 1. In another aspect, the amino acid at a position corresponding to position 172 is substituted with Ala, Arg, Asn, Asp, Cys, Gln, Glu, Gly, His, lie, Leu, Met, Phe, Pro, Ser, Thr, Trp, Tyr, or Val. In another aspect, the variant comprises or consists of the substitution K172G or K172Q of the polypeptide of SEQ ID NO: 1.

In another aspect, the variant comprises or consists of a substitution at a position corresponding to position 173 of SEQ ID NO: 1. In another aspect, the amino acid at a position corresponding to position 173 is substituted with Ala, Arg, Asn, Asp, Cys, Gln, Glu, Gly, His, lie, Lys, Met, Phe, Pro, Ser, Thr, Trp, Tyr, or Val. In another aspect, the variant comprises or consists of the substitution L173F of the polypeptide of SEQ ID NO: 1.

In another aspect, the variant comprises or consists of a substitution at a position corresponding to position 174 of SEQ ID NO: 1. In another aspect, the amino acid at a position corresponding to position 174 is substituted with Ala, Arg, Asp, Cys, Gln, Glu, Gly, His, lie, Leu, Lys, Met, Phe, Pro, Ser, Thr, Trp, Tyr, or Val. In another aspect, the variant comprises or consists of the substitution N174K or N174S of the polypeptide of SEQ ID NO: 1.

In another aspect, the variant comprises or consists of a substitution at a position corresponding to position 176 of SEQ ID NO: 1. In another aspect, the amino acid at a position corresponding to position 176 is substituted with Ala, Arg, Asn, Asp, Cys, Gln, Glu, Gly, His, Leu, Lys, Met, Phe, Pro, Ser, Thr, Trp, Tyr, or Val. In another aspect, the variant comprises or consists of the substitution I176V of the polypeptide of SEQ ID NO: 1.

In another aspect, the variant comprises or consists of a substitution at a position corresponding to position 177 of SEQ ID NO: 1. In another aspect, the amino acid at a position corresponding to position 177 is substituted with Ala, Arg, Asp, Cys, Gln, Glu, Gly, His, lie, Leu, Lys, Met, Phe, Pro, Ser, Thr, Trp, or Val. In another aspect, the variant comprises or consists of the substitution Y177D of the polypeptide of SEQ ID NO: 1.

In another aspect, the variant comprises or consists of a substitution at a position corresponding to position 178 of SEQ ID NO: 1. In another aspect, the amino acid at a position corresponding to position 178 is substituted with Ala, Arg, Asn, Asp, Cys, Gln, Glu, Gly, His, lie, Lys, Met, Phe, Pro, Ser, Thr, Trp, Tyr, or Val. In another aspect, the variant comprises or consists of the substitution K178A of the polypeptide of SEQ ID NO: 1.

In another aspect, the variant comprises or consists of a deletion or substitution at a position corresponding to position 180. In another aspect, the amino acid at a position corresponding to position 180 is deleted of the polypeptide of SEQ ID NO: 1. In another aspect, the variant comprises or consists of deletion R180* of the polypeptide of SEQ ID NO: 1. In another aspect, the amino acid at a position corresponding to position 180 is substituted with Ala, Asn, Asp, Cys, Gln, Glu, Gly, His, lie, Leu, Lys, Met, Phe, Pro, Ser, Thr, Trp, Tyr, or Val. In another aspect, the variant comprises or consists of the substitution R180Q or R180S of the polypeptide of SEQ ID NO: 1.

In another aspect, the variant comprises or consists of a deletion or substitution at a position corresponding to position 181. In another aspect, the amino acid at a position corresponding to position 181 is deleted of the polypeptide of SEQ ID NO: 1. In another aspect, the variant comprises or consists of deletion S181* of the polypeptide of SEQ ID NO: 1. In another aspect, the amino acid at a position corresponding to position 181 is substituted with Ala, Arg, Asn, Asp, Cys, Gln, Glu, Gly, His, lie, Leu, Lys, Met, Phe, Pro, Thr, Trp, Tyr, or Val. In another aspect, the variant comprises or consists of the substitution S181L or S181T of the polypeptide of SEQ ID NO: 1.

In another aspect, the variant comprises or consists of deletion or substitution at a position corresponding to position 182 of SEQ ID NO: 1. In another aspect, the amino acid at a position corresponding to position 182 is deleted of the polypeptide of SEQ ID NO: 1. In another aspect, the variant comprises or consists of deletion T182* of the polypeptide of SEQ ID NO: 1. In another aspect, the amino acid at a position corresponding to position 182 is substituted with Ala, Arg, Asn, Asp, Cys, Gln, Glu, Gly, His, lie, Leu, Lys, Met, Phe, Pro, Ser, Trp, Tyr, or Val. In another aspect, the variant comprises or consists of the substitution T182D or T182E or T182G or T182I or T182L or T182M or T182S of the polypeptide of SEQ ID NO: 1.

In another aspect, the variant comprises or consists of deletion or substitution at a position corresponding to position 183 of SEQ ID NO: 1. In another aspect, the amino acid at a position corresponding to position 183 is deleted of the polypeptide of SEQ ID NO: 1. In another aspect, the variant comprises or consists of deletion G183* of the polypeptide of SEQ ID NO: 1. In another aspect, the amino acid at a position corresponding to position 183 is substituted with Ala, Arg, Asn, Asp, Cys, Gln, Glu, His, Ile, Leu, Lys, Met, Phe, Pro, Ser, Thr, Trp, Tyr, or Val. In another aspect, the variant comprises or consists of the substitution G183P of the polypeptide of SEQ ID NO: 1.

In another aspect, the variant comprises or consists of a substitution at a position corresponding to position 185 of SEQ ID NO: 1. In another aspect, the amino acid at a position corresponding to position 185 is substituted with Arg, Asn, Asp, Cys, Gln, Glu, Gly, His, Ile, Leu, Lys, Met, Phe, Pro, Ser, Thr, Trp, Tyr, or Val. In another aspect, the variant comprises or consists of the substitution A186D or A186E or A186K or A186N or A186Q or A186R of the polypeptide of SEQ ID NO: 1.

In another aspect, the variant comprises or consists of a substitution at a position corresponding to position 188 of SEQ ID NO: 1. In another aspect, the amino acid at a position corresponding to position 188 is substituted with Ala, Arg, Asn, Asp, Cys, Gln, Glu, Gly, His, Ile, Leu, Lys, Met, Phe, Pro, Ser, Thr, Tyr, or Val. In another aspect, the variant comprises or consists of the substitution W188C of the polypeptide of SEQ ID NO: 1.

In another aspect, the variant comprises or consists of a substitution at a position corresponding to position 189 of SEQ ID NO: 1. In another aspect, the amino acid at a position corresponding to position 189 is substituted with Ala, Arg, Asn, Asp, Cys, Gln, Gly, His, Ile, Leu, Lys, Met, Phe, Pro, Ser, Thr, Trp, Tyr, or Val. In another aspect, the variant comprises or consists of the substitution E189P of the polypeptide of SEQ ID NO: 1.

In another aspect, the variant comprises or consists of a substitution at a position corresponding to position 193 of SEQ ID NO: 1. In another aspect, the amino acid at a position corresponding to position 193 is substituted with Ala, Arg, Asn, Asp, Cys, Gln, Gly, His, Ile, Leu, Lys, Met, Phe, Pro, Ser, Thr, Trp, Tyr, or Val. In another aspect, the variant comprises or consists of the substitution E193I of the polypeptide of SEQ ID NO: 1.

In another aspect, the variant comprises or consists of a substitution at a position corresponding to position 194 of SEQ ID NO: 1. In another aspect, the amino acid at a position corresponding to position 194 is substituted with Ala, Arg, Asp, Cys, Gln, Glu, Gly, His, Ile, Leu, Lys, Met, Phe, Pro, Ser, Thr, Trp, Tyr, or Val. In another aspect, the variant comprises or consists of the substitution N194F of the polypeptide of SEQ ID NO: 1.

In another aspect, the variant comprises or consists of a substitution at a position corresponding to position 196 of SEQ ID NO: 1. In another aspect, the amino acid at a position corresponding to position 196 is substituted with Ala, Arg, Asp, Cys, Gln, Glu, Gly, His, lie, Leu, Lys, Met, Phe, Pro, Ser, Thr, Trp, Tyr, or Val. In another aspect, the variant comprises or consists of the substitution N196F or N196I of the polypeptide of SEQ ID NO: 1.

In another aspect, the variant comprises or consists of a substitution at a position corresponding to position 202 of SEQ ID NO: 1. In another aspect, the amino acid at a position corresponding to position 202 is substituted with Ala, Arg, Asn, Asp, Cys, Gln, Glu, Gly, His, lie, Leu, Lys, Met, Pro, Ser, Thr, Trp, Tyr, or Val. In another aspect, the variant comprises or consists of the substitution F202Y of the polypeptide of SEQ ID NO: 1.

In another aspect, the variant comprises or consists of a substitution at a position corresponding to position 203 of SEQ ID NO: 1. In another aspect, the amino acid at a position corresponding to position 203 is substituted with Arg, Asn, Asp, Cys, Gln, Glu, Gly, His, lie, Leu, Lys, Met, Phe, Pro, Ser, Thr, Trp, Tyr, or Val. In another aspect, the variant comprises or consists of the substitution A203F of the polypeptide of SEQ ID NO: 1.

In another aspect, the variant comprises or consists of a substitution at a position corresponding to position 205 of SEQ ID NO: 1. In another aspect, the amino acid at a position corresponding to position 205 is substituted with Ala, Arg, Asn, Asp, Cys, Gln, Glu, Gly, His, lie, Lys, Met, Phe, Pro, Ser, Thr, Trp, Tyr, or Val. In another aspect, the variant comprises or consists of the substitution L205F or L205N or L205Q or L205R or L205V or L205Y of the polypeptide of SEQ ID NO: 1.

In another aspect, the variant comprises or consists of a substitution at a position corresponding to position 224 of SEQ ID NO: 1. In another aspect, the amino acid at a position corresponding to position 224 is substituted with Ala, Arg, Asn, Asp, Cys, Gln, Glu, Gly, His, lie, Leu, Lys, Met, Phe, Pro, Ser, Trp, Tyr, or Val. In another aspect, the variant comprises or consists of the substitution T224F or T244Y of the polypeptide of SEQ ID NO: 1.

In another aspect, the variant comprises or consists of a substitution at a position corresponding to position 225 of SEQ ID NO: 1. In another aspect, the amino acid at a position corresponding to position 225 is substituted with Ala, Arg, Asp, Cys, Gln, Glu, Gly, His, lie, Leu, Lys, Met, Phe, Pro, Ser, Thr, Trp, Tyr, or Val. In another aspect, the variant comprises or consists of the substitution N225S of the polypeptide of SEQ ID NO: 1.

In another aspect, the variant comprises or consists of a substitution at a position corresponding to position 242 of SEQ ID NO: 1. In another aspect, the amino acid at a position corresponding to position 242 is substituted with Ala, Arg, Asn, Asp, Cys, Gln, Glu, Gly, His, lie, Leu, Lys, Met, Phe, Pro, Ser, Thr, Trp, or Val. In another aspect, the variant comprises or consists of the substitution Y242F or Y242L of the polypeptide of SEQ ID NO: 1.

In another aspect, the variant comprises or consists of a substitution at a position corresponding to position 243 of SEQ ID NO: 1. In another aspect, the amino acid at a position corresponding to position 243 is substituted with Ala, Arg, Asn, Asp, Cys, Gln, Glu, Gly, His, lie, Leu, Lys, Met, Phe, Pro, Thr, Trp, Tyr, or Val. In another aspect, the variant comprises or consists of the substitution S243Q of the polypeptide of SEQ ID NO: 1.

In another aspect, the variant comprises or consists of a substitution at a position corresponding to position 244 of SEQ ID NO: 1. In another aspect, the amino acid at a position corresponding to position 244 is substituted with Ala, Arg, Asn, Asp, Cys, Gln, Glu, Gly, His, lie, Leu, Lys, Met, Ser, Pro, Thr, Trp, Tyr, or Val. In another aspect, the variant comprises or consists of the substitution F244I or F244L of the polypeptide of SEQ ID NO: 1.

In another aspect, the variant comprises or consists of a substitution at a position corresponding to position 252 of SEQ ID NO: 1. In another aspect, the amino acid at a position corresponding to position 252 is substituted with Ala, Arg, Asn, Asp, Cys, Gln, Glu, Gly, His, lie, Leu, Lys, Met, Phe, Pro, Ser, Thr, Trp, or Tyr. In another aspect, the variant comprises or consists of the substitution V252I of the polypeptide of SEQ ID NO: 1.

In another aspect, the variant comprises or consists of a substitution at a position corresponding to position 256 of SEQ ID NO: 1. In another aspect, the amino acid at a position corresponding to position 256 is substituted with Ala, Arg, Asn, Asp, Cys, Gln, Glu, Gly, His, lie, Leu, Lys, Met, Phe, Pro, Ser, Trp, Tyr, or Val. In another aspect, the variant comprises or consists of the substitution T256A or T256D of the polypeptide of SEQ ID NO: 1.

In another aspect, the variant comprises or consists of a substitution at a position corresponding to position 258. In another aspect, the amino acid at a position corresponding to position 258 is substituted with Ala, Arg, Asn, Asp, Cys, Gln, Glu, Gly, His, lie, Leu, Met, Phe, Pro, Ser, Thr, Trp, Tyr, or Val. In another aspect, the variant comprises or consists of the substitution K258V of the polypeptide of SEQ ID NO: 1.

In another aspect, the variant comprises or consists of a substitution at a position corresponding to position 259. In another aspect, the amino acid at a position corresponding to position 259 is substituted with Ala, Arg, Asn, Asp, Cys, Gln, Gly, His, lie, Leu, Lys, Met, Phe, Pro, Ser, Thr, Trp, Tyr, or Val. In another aspect, the variant comprises or consists of the substitution E259G or E259V of the polypeptide of SEQ ID NO: 1.

In another aspect, the variant comprises or consists of a substitution at a position corresponding to position 260. In another aspect, the amino acid at a position corresponding to position 260 is substituted with Ala, Arg, Asn, Asp, Cys, Glu, Gln, Gly, His, lie, Leu, Lys, Phe, Pro, Ser, Thr, Trp, Tyr, or Val. In another aspect, the variant comprises or consists of the substitution M260D or M260W of the polypeptide of SEQ ID NO: 1.

In another aspect, the variant comprises or consists of a substitution at a position corresponding to position 261. In another aspect, the amino acid at a position corresponding to position 261 is substituted with Ala, Arg, Asn, Asp, Cys, Gln, Glu, Gly, His, lie, Leu, Lys, Met, Pro, Ser, Thr, Trp, Tyr, or Val. In another aspect, the variant comprises or consists of the substitution F261K or F261P or F261S of the polypeptide of SEQ ID NO: 1.

In another aspect, the variant comprises or consists of a substitution at a position corresponding to position 266. In another aspect, the amino acid at a position corresponding to position 266 is substituted with Ala, Arg, Asn, Asp, Cys, Gln, Glu, Gly, His, lie, Leu, Lys, Met, Pro, Ser, Thr, Trp, Tyr, or Val. In another aspect, the variant comprises or consists of the substitution F266Y of the polypeptide of SEQ ID NO: 1.

In another aspect, the variant comprises or consists of a substitution at a position corresponding to position 276. In another aspect, the amino acid at a position corresponding to position 276 is substituted with Ala, Arg, Asp, Cys, Gln, Glu, Gly, His, lie, Leu, Lys, Met, Pro, Phe, Ser, Thr, Trp, Tyr, or Val. In another aspect, the variant comprises or consists of the substitution N276F or N276W or N276Y of the polypeptide of SEQ ID NO: 1.

In another aspect, the variant comprises or consists of a substitution at a position corresponding to position 279. In another aspect, the amino acid at a position corresponding to position 279 is substituted with Ala, Arg, Asp, Cys, Gln, Glu, Gly, His, lie, Leu, Lys, Met, Pro, Phe, Ser, Thr, Trp, Tyr, or Val. In another aspect, the variant comprises or consists of the substitution N279S of the polypeptide of SEQ ID NO: 1.

In another aspect, the variant comprises or consists of a substitution at a position corresponding to position 280. In another aspect, the amino acid at a position corresponding to position 280 is substituted with Ala, Arg, Asn, Asp, Cys, Gln, Glu, Gly, His, lie, Leu, Met, Pro, Phe, Ser, Thr, Trp, Tyr, or Val. In another aspect, the variant comprises or consists of the substitution K280F or K280W or K280Y of the polypeptide of SEQ ID NO: 1.

In another aspect, the variant comprises or consists of a substitution at a position corresponding to position 283. In another aspect, the amino acid at a position corresponding to position 283 is substituted with Ala, Arg, Asn, Asp, Cys, Gln, Glu, Gly, His, lie, Leu, Lys, Met, Pro, Phe, Ser, Thr, Tyr, or Val. In another aspect, the variant comprises or consists of the substitution W283N of the polypeptide of SEQ ID NO: 1.

In another aspect, the variant comprises or consists of a substitution at a position corresponding to position 286. In another aspect, the amino acid at a position corresponding to position 286 is substituted with Ala, Arg, Asn, Asp, Cys, Gln, Glu, Gly, His, lie, Leu, Lys, Met, Pro, Phe, Thr, Trp, Tyr, or Val. In another aspect, the variant comprises or consists of the substitution S286K or S286L of the polypeptide of SEQ ID NO: 1.

In another aspect, the variant comprises or consists of a substitution at a position corresponding to position 302. In another aspect, the amino acid at a position corresponding to position 302 is substituted with Ala, Arg, Asn, Asp, Cys, Gln, Glu, Gly, His, lie, Leu, Lys, Met, Pro, Phe, Thr, Trp, Tyr, or Val. In another aspect, the variant comprises or consists of the substitution S302T or S302Y of the polypeptide of SEQ ID NO: 1.

In another aspect, the variant comprises or consists of a substitution at a position corresponding to position 303. In another aspect, the amino acid at a position corresponding to position 303 is substituted with Ala, Arg, Asn, Asp, Cys, Gln, Glu, His, lie, Leu, Lys, Met, Pro, Phe, Ser, Thr, Trp, Tyr, or Val. In another aspect, the variant comprises or consists of the substitution G303R of the polypeptide of SEQ ID NO: 1.

In another aspect, the variant comprises or consists of a substitution at a position corresponding to position 315. In another aspect, the amino acid at a position corresponding to position 315 is substituted with Ala, Arg, Asn, Asp, Cys, Gln, Glu, Gly, His, lie, Leu, Lys, Met, Pro, Phe, Ser, Trp, Tyr, or Val. In another aspect, the variant comprises or consists of the substitution T315A of the polypeptide of SEQ ID NO: 1.

In another aspect, the variant comprises or consists of a substitution at a position corresponding to position 320. In another aspect, the amino acid at a position corresponding to position 320 is substituted with Ala, Arg, Asn, Asp, Cys, Gln, Glu, Gly, lie, Leu, Lys, Met, Pro, Phe, Ser, Thr, Trp, Tyr, or Val. In another aspect, the variant comprises or consists of the substitution H320T of the polypeptide of SEQ ID NO: 1.

In another aspect, the variant comprises or consists of a substitution at a position corresponding to position 321. In another aspect, the amino acid at a position corresponding to position 321 is substituted with Ala, Arg, Asn, Asp, Cys, Gln, Glu, Gly, His, lie, Leu, Lys, Met, Phe, Ser, Thr, Trp, Tyr, or Val. In another aspect, the variant comprises or consists of the substitution P321H or P321Y of the polypeptide of SEQ ID NO: 1.

In another aspect, the variant comprises or consists of a substitution at a position corresponding to position 322. In another aspect, the amino acid at a position corresponding to position 322 is substituted with Ala, Arg, Asn, Asp, Cys, Gln, Glu, Gly, His, lie, Leu, Lys, Pro, Phe, Ser, Thr, Trp, Tyr, or Val. In another aspect, the variant comprises or consists of the substitution M322G or M322Y of the polypeptide of SEQ ID NO: 1.

In another aspect, the variant comprises or consists of a substitution at a position corresponding to position 323. In another aspect, the amino acid at a position corresponding to position 323 is substituted with Ala, Arg, Asn, Asp, Cys, Gln, Glu, Gly, lie, Leu, Lys, Met, Pro, Phe, Ser, Thr, Trp, Tyr, or Val. n another aspect, the variant comprises or consists of the substitution H323A or H323N or H323W of the polypeptide of SEQ ID NO: 1.

In another aspect, the variant comprises or consists of a substitution at a position corresponding to position 346. In another aspect, the amino acid at a position corresponding to position 346 is substituted with Ala, Arg, Asn, Asp, Cys, Gln, Glu, Gly, His, lie, Leu, Lys, Met, Pro, Phe, Ser, Thr, Tyr, or Val. In another aspect, the variant comprises or consists of the substitution W346N of the polypeptide of SEQ ID NO: 1.

In another aspect, the variant comprises or consists of a substitution at a position corresponding to position 354. In another aspect, the amino acid at a position corresponding to position 354 is substituted with Ala, Arg, Asn, Asp, Cys, Gln, Glu, Gly, His, lie, Lys, Met, Pro, Phe, Ser, Thr, Trp, Tyr, or Val. In another aspect, the variant comprises or consists of the substitution L354V of the polypeptide of SEQ ID NO: 1.

In another aspect, the variant comprises or consists of a substitution at a position corresponding to position 359. In another aspect, the amino acid at a position corresponding to position 359 is substituted with Ala, Arg, Asn, Asp, Cys, Gln, Gly, His, lie, Leu, Lys, Met, Pro, Phe, Ser, Thr, Trp, Tyr, or Val. In another aspect, the variant comprises or consists of the substitution E359W of the polypeptide of SEQ ID NO: 1.

In another aspect, the variant comprises or consists of a substitution at a position corresponding to position 360. In another aspect, the amino acid at a position corresponding to position 360 is substituted with Ala, Arg, Asn, Asp, Cys, Glu, Gly, His, lie, Leu, Lys, Met, Pro, Phe, Ser, Thr, Trp, Tyr, or Val. In another aspect, the variant comprises or consists of the substitution Q360M or Q360W or Q360Y of the polypeptide of SEQ ID NO: 1.

In another aspect, the variant comprises or consists of a substitution at a position corresponding to position 361. In another aspect, the amino acid at a position corresponding to position 361 is substituted with Ala, Arg, Asn, Asp, Cys, Gln, Glu, His, lie, Leu, Lys, Met, Phe, Pro, Ser, Thr, Trp, or Tyr. In another aspect, the variant comprises or consists of the substitution G361M of the polypeptide of SEQ ID NO: 1.

In another aspect, the variant comprises or consists of a substitution at a position corresponding to position 362. In another aspect, the amino acid at a position corresponding to position 362 is substituted with Ala, Arg, Asn, Asp, Cys, Gln, Glu, Gly, His, lie, Leu, Lys, Met, Pro, Phe, Ser, Thr, Trp, or Val. In another aspect, the variant comprises or consists of the substitution Y362V of the polypeptide of SEQ ID NO: 1.

In another aspect, the variant comprises or consists of a substitution at a position corresponding to position 365. In another aspect, the amino acid at a position corresponding to position 365 is substituted with Ala, Arg, Asn, Asp, Cys, Gln, Glu, Gly, His, lie, Leu, Lys, Met, Pro, Phe, Ser, Thr, Tyr, or Trp. In another aspect, the variant comprises or consists of the substitution V365I of the polypeptide of SEQ ID NO: 1.

In another aspect, the variant comprises or consists of a substitution at a position corresponding to position 371. In another aspect, the amino acid at a position corresponding to position 371 is substituted with Ala, Arg, Asn, Asp, Cys, Gln, Glu, Gly, His, lie, Leu, Lys, Met, Pro, Phe, Ser, Thr, Trp, or Val. In another aspect, the variant comprises or consists of the substitution Y371S or Y371N of the polypeptide of SEQ ID NO: 1.

In another aspect, the variant comprises or consists of a substitution at a position corresponding to position 383. In another aspect, the amino acid at a position corresponding to position 383 is substituted with Arg, Asn, Asp, Cys, Gln, Glu, Gly, His, lie, Leu, Lys, Met, Pro, Phe, Ser, Thr, Trp, Tyr, or Val. In another aspect, the variant comprises or consists of the substitution A383D of the polypeptide of SEQ ID NO: 1.

In another aspect, the variant comprises or consists of a substitution at a position corresponding to position 390. In another aspect, the amino acid at a position corresponding to position 390 is substituted with Ala, Arg, Asn, Asp, Cys, Gln, Gly, His, lie, Leu, Lys, Met, Pro, Phe, Ser, Thr, Trp, Tyr, or Val. In another aspect, the variant comprises or consists of the substitution E390A or E390D of the polypeptide of SEQ ID NO: 1.

In another aspect, the variant comprises or consists of a substitution at a position corresponding to position 394. In another aspect, the amino acid at a position corresponding to position 394 is substituted with Ala, Arg, Asp, Cys, Gln, Glu, Gly, His, lie, Leu, Lys, Met, Pro, Phe, Ser, Thr, Trp, Tyr, or Val. In another aspect, the variant comprises or consists of the substitution N394W or N394D of the polypeptide of SEQ ID NO: 1.

In another aspect, the variant comprises or consists of a substitution at a position corresponding to position 395. In another aspect, the amino acid at a position corresponding to position 395 is substituted with Ala, Arg, Asn, Asp, Cys, Gln, Glu, Gly, His, lie, Leu, Lys, Met, Pro, Ser, Thr, Trp, Tyr, or Val. In another aspect, the variant comprises or consists of the substitution F395Y of the polypeptide of SEQ ID NO: 1.

In another aspect, the variant comprises or consists of a substitution at a position corresponding to position 405. In another aspect, the amino acid at a position corresponding to position 405 is substituted with Ala, Arg, Asn, Cys, Gln, Glu, Gly, His, lie, Leu, Lys, Met, Pro, Phe, Ser, Thr, Trp, Tyr, or Val. In another aspect, the variant comprises or consists of the substitution D405G of the polypeptide of SEQ ID NO: 1.

In another aspect, the variant comprises or consists of a substitution at a position corresponding to position 409. In another aspect, the amino acid at a position corresponding to position 409 is substituted with Ala, Arg, Asn, Asp, Cys, Gln, Glu, Gly, His, Leu, Lys, Met, Pro, Phe, Ser, Thr, Trp, Tyr, or Val. In another aspect, the variant comprises or consists of the substitution I409L of the polypeptide of SEQ ID NO: 1.

In another aspect, the variant comprises or consists of a substitution at a position corresponding to position 414. In another aspect, the amino acid at a position corresponding to position 414 is substituted with Ala, Asn, Asp, Cys, Gln, Glu, Gly, His, lie, Leu, Lys, Met, Phe, Pro, Ser, Thr, Trp, Tyr, or Val. In another aspect, the variant comprises or consists of the substitution R414M of the polypeptide of SEQ ID NO: 1.

In another aspect, the variant comprises or consists of a substitution at a position corresponding to position 420. In another aspect, the amino acid at a position corresponding to position 420 is substituted with Ala, Arg, Asn, Asp, Cys, Gln, Glu, Gly, lie, Leu, Lys, Met, Pro, Phe, Ser, Thr, Trp, Tyr, or Val. In another aspect, the variant comprises or consists of the substitution H420A or H420P or H420Q or H420Y of the polypeptide of SEQ ID NO: 1.

In another aspect, the variant comprises or consists of a substitution at a position corresponding to position 422. In another aspect, the amino acid at a position corresponding to position 422 is substituted with Ala, Arg, Asn, Asp, Cys, Gln, Glu, Gly, His, lie, Leu, Lys, Met, Phe, Pro, Ser, Thr, Trp or Tyr. In another aspect, the variant comprises or consists of the substitution N422V of the polypeptide of SEQ ID NO: 1.

In another aspect, the variant comprises or consists of a substitution at a position corresponding to position 427. In another aspect, the amino acid at a position corresponding to position 427 is substituted with Ala, Arg, Asn, Asp, Cys, Gln, Glu, Gly, His, lie, Leu, Lys, Met, Pro, Phe, Ser, Trp, Tyr, or Val. In another aspect, the variant comprises or consists of the substitution T427L of the polypeptide of SEQ ID NO: 1.

In another aspect, the variant comprises or consists of a substitution at a position corresponding to position 455. In another aspect, the amino acid at a position corresponding to position 455 is substituted with Ala, Arg, Asn, Asp, Cys, Gln, Glu, His, lie, Leu, Lys, Met, Pro, Phe, Ser, Thr, Trp, Tyr, or Val. In another aspect, the variant comprises or consists of the substitution G455R of the polypeptide of SEQ ID NO: 1.

In another aspect, the variant comprises or consists of a substitution at a position corresponding to position 457. In another aspect, the amino acid at a position corresponding to position 457 is substituted with Ala, Arg, Asn, Asp, Cys, Gln, Glu, Gly, His, lie, Leu, Met, Pro, Phe, Ser, Thr, Trp, Tyr, or Val. In another aspect, the variant comprises or consists of the substitution K457N or K457R of the polypeptide of SEQ ID NO: 1.

In another aspect, the variant comprises or consists of a substitution at a position corresponding to position 458. In another aspect, the amino acid at a position corresponding to position 458 is substituted with Ala, Arg, Asn, Asp, Cys, Gln, Glu, Gly, His, lie, Leu, Lys, Met, Phe, Ser, Thr, Trp, Tyr, or Val. In another aspect, the variant comprises or consists of the substitution P458S of the polypeptide of SEQ ID NO: 1.

In another aspect, the variant comprises or consists of a substitution at a position corresponding to position 470. In another aspect, the amino acid at a position corresponding to position 470 is substituted with Ala, Arg, Asp, Cys, Gln, Glu, Gly, His, lie, Leu, Lys, Met, Pro, Phe, Ser, Thr, Trp, Tyr, or Val. In another aspect, the variant comprises or consists of the substitution N470A or N470M of the polypeptide of SEQ ID NO: 1.

In another aspect, the variant comprises or consists of a substitution at a position corresponding to position 472. In another aspect, the amino acid at a position corresponding to position 472 is substituted with Ala, Arg, Asn, Asp, Cys, Gln, Glu, Gly, His, lie, Leu, Lys, Met, Pro, Phe, Thr, Trp, Tyr, or Val. In another aspect, the variant comprises or consists of the substitution S472R of the polypeptide of SEQ ID NO: 1.

In another aspect, the variant comprises or consists of a substitution at a position corresponding to position 473. In another aspect, the amino acid at a position corresponding to position 473 is substituted with Ala, Arg, Asn, Asp, Cys, Gln, Glu, Gly, His, lie, Leu, Lys, Met, Pro, Phe, Ser, Thr, Trp, or Tyr. In another aspect, the variant comprises or consists of the substitution V473C of the polypeptide of SEQ ID NO: 1.

In another aspect, the variant comprises or consists of a substitution at a position corresponding to position 474. In another aspect, the amino acid at a position corresponding to position 474 is substituted with Ala, Arg, Asp, Cys, Gln, Glu, Gly, His, lie, Leu, Lys, Met, Pro, Phe, Ser, Thr, Trp, Tyr, or Val. In another aspect, the variant comprises or consists of the substitution N474F or N474L of the polypeptide of SEQ ID NO: 1.

In another aspect, the variant comprises or consists of a substitution at a position corresponding to position 475. In another aspect, the amino acid at a position corresponding to position 475 is substituted with Ala, Arg, Asn, Asp, Cys, Gln, Glu, His, lie, Leu, Lys, Met, Pro, Phe, Ser, Thr, Trp, Tyr, or Val. In another aspect, the variant comprises or consists of the substitution G475E or G475K of the polypeptide of SEQ ID NO: 1.

In another aspect, the variant comprises or consists of a substitution at a position corresponding to position 476. In another aspect, the amino acid at a position corresponding to position 476 is substituted with Ala, Arg, Asn, Asp, Cys, Gln, Glu, His, lie, Leu, Lys, Met, Phe, Pro, Ser, Thr, Trp, Tyr, or Val. In another aspect, the variant comprises or consists of the substitution G476A or G476E or G476H of the polypeptide of SEQ ID NO: 1.

In another aspect, alpha-amylase variant comprises a modification at one or more positions corresponding to positions: H1, H2, N3, G7, R26, A31, R37, T40, A41, N54, V56, A60, L63, K72, G73, K93, N94, Q98, V103, V104, M105, N106, A109, G110, A111, G113, T114, F116, V117, D118, D123, N126, N128, T131, S132, T134, Y135, Q136, I137, Q138, W140, K142, D144, P146, T151, Y152, F155, W159, Y160, T165, W167, E169, K172, L173, N174, I176, Y177, K178, R180, S181, T182, G183, A185, W188, E189, E193, N194, N196, F202, A203, L205, T224, N225, Y242, S243, F244, V252, T256, K258, E259, M260, F261, F266, N276, N279, K280, W283, S286, S302, G303, T315, H320, P321, M322, H323, W346, L354, E359, Q360, G361, Y362, V365, Y371, A383, E390, N394, F395, D405, I409, R414, H420, N422, T427, G455, K457, P458, N470, S472, V473, N474, G475 and G476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 2.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 3.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 4.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 5.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 6.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 7.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 8.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 9.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 10.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 11.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 12.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 13.

In another aspect, alpha-amylase variant comprises a modification at one or more positions corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 14.

The term “pairwise deletion” as used herein, refers to a deletion of an amino acid in two positions. Said two positions may be adjacent to one another but may also be separated by one, two, three, four, or five amino acids.

In one aspect, a variant comprises a pairwise deletion within the B domain of parent polypeptide.

In one aspect, a variant comprises a pairwise deletion within the B domain of parent polypeptide having alpha-amylase activity.

The term “pairwise deletion” as used herein, refers to a deletion of an amino acid in two positions. Said two positions may be adjacent to one another but may also be separated by one, two, three, four, or five amino acids.

In one aspect, the alpha-amylase variant of the present invention comprises deletion at one or more position selected from the group consisting of R180*+S181*, R180*+T182*, R180*+G183*, S181*+T182*, S181*+G183* and T182*+G183*, preferably R180*+S181*, using SEQ ID NO: 1 for numbering.

In one aspect, the invention relates to variant of SEQ ID NO: 1 comprising deletion in the positions corresponding to R180*+S181*, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14.

Thus, in one embodiment, the said alpha-amylase variant comprises

a) a deletion and/or a substitution at two or more positions corresponding to positions 180, 181, 182 and 183, using SEQ ID NO: 1 for numbering, and
b) a modification at one or more positions corresponding to positions 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one embodiment, the said alpha-amylase variant comprises

a) a deletion and/or a substitution at two or more positions corresponding to positions R180, S181, T182 and G183, using SEQ ID NO: 1 for numbering, and
b) a modification at one or more positions corresponding to positions H1, H2*, N3, G7, R26, A31, R37, T40, A41, N54, V56, A60, L63, K72, G73, K93, N94, Q98, V103, V104, M105, N106, A109, G110, A111, G113, T114, F116, V117, D118, D123, N126, N128, T131, S132, T134, Y135, Q136, 1137, Q138, W140, K142, D144, P146, T151, Y152, F155, W159, Y160, T165, W167, E169, K172, L173, N174, I176, Y177, K178, A185, W188, E189, E193, N194, N196, F202, A203, L205, T224, N225, Y242, S243, F244, V252, T256, K258, E259, M260, F261, F266, N276, N279, K280, W283, S286, S302, G303, T315, H320, P321, M322, H323, W346, L354, E359, Q360, G361, Y362, V365, Y371, A383, E390, N394, F395, D405, I409, R414, H420, N422, T427, G455, K457, P458, N470, S472, V473, N474, G475 and G476, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one embodiment, the said alpha-amylase variant comprises
a) a deletion and/or a substitution at two or more positions corresponding to positions R180, S181, T182 and G183, using SEQ ID NO: 1 for numbering, and
b) a modification at one or more positions corresponding to positions H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 1 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D118T+W140Y+A185D, D118T+W140Y+E189P, D118T+W140Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the invention relates to alpha-amylase variant of SEQ ID NO: 1 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1*+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1*+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 1 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 1 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1**+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y, G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 1 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 1 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 1 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 1 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 2 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G, T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 2 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D118T+W140Y+A185D, D118T+W140Y+E189P, D118T+W140Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 2 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1*+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1*+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 2 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 2 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1*+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y, G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the variant of SEQ ID NO: 2 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 2 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 2 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 2 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 3 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G, T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 3 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D118T+W140Y+A185D, D118T+W140Y+E189P, D118T+W140Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K, K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 3 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1*+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1*+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 3 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A, G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 3 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1**+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y, G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 3 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 3 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 3 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 3 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 4 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D 118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G, T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 4 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D118T+W140Y+A185D, D118T+W140Y+E189P, D118T+W140Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K, K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 4 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1*+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1*+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 4 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 4 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1*+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y, G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 4 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 4 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 4 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 4 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 5 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D 118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G, T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 5 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D118T+W140Y+A185D, D118T+W140Y+E189P, D118T+W140Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K, K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 5 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1**+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1**+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 5 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A, G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 5 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1**+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 5 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 5 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 5 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 5 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 6 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G, T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 6 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D 118T+W140Y+A185D, D118T+W140Y+E189P, D118T+W140Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K, K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 6 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1*+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1*+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 6 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A, G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 6 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1*+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y, G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 6 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 6 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 6 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 6 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 7 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G, T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 7 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D118T+W140Y+A185D, D118T+W140Y+E189P, D118T+W140Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K, K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 7 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1*+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1*+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 7 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A, G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 7 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1*+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y, G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 7 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 7 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 7 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 7 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 8 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G, T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 8 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D118T+W140Y+A185D, D118T+W140Y+E189P, D118T+W40Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K, K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 8 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1*+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1*+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 8 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A, G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 8 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1*+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 8 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 8 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 8 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 8 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 9 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G, T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 9 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D118T+W140Y+A185D, D118T+W140Y+E189P, D118T+W140Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 9 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1*+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1*+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 9 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A, G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 9 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1**+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y, G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 9 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 9 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 9 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 9 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 10 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G, T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 10 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D118T+W140Y+A185D, D118T+W140Y+E189P, D118T+W140Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K, K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 10 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1*+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1*+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 10 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A, G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 10 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1*+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y, G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, alpha-amylase variant of SEQ ID NO: 10 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 10 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 10 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 10 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 11 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G, T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 11 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D118T+W140Y+A185D, D118T+W140Y+E189P, D118T+W140Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 11 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1*+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1*+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 11 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A, G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 11 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1*+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y, G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 11 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 11 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 11 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 11 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 12 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G, T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 12 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D118T+W140Y+A185D, D118T+W140Y+E189P, D118T+W140Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 12 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1*+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1**+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 12 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A, G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 12 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1**+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y, G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 12 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 12 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 12 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 12 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 13 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G, T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 13 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D118T+W140Y+A185D, D118T+W140Y+E189P, D118T+W140Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K, K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 13 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1*+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1*+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 13 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A, G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 13 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1*+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y, G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 13 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 13 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 13 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 13 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 14 comprising:

a) deletion at positions corresponding to positions R180* and S181*, using SEQ ID NO: 1 for numbering, and

b) modification at positions corresponding to positions: H1*+D118T, H1*+W140Y, H1*+T182S, H1*+A185D, H1*+E189P, H1*+Y242F, H1*+E259G, H1*+F266Y, H1*+N279S, H1*+K280Y, H1*+G303R, H1*+E390A, H1*+G475K, H1*+A185Q, H1*+N194F, D118T+W140Y, D118T+T182S, D118T+A185D, D118T+E189P, D118T+Y242F, D118T+E259G, D118T+F266Y, D118T+N279S, D118T+K280Y, D118T+G303R, D118T+E390A, D118T+G475K, D118T+A185Q, D118T+N194F, W140Y+T182S, W140Y+A185D, W140Y+E189P, W140Y+Y242F, W140Y+E259G, W140Y+F266Y, W140Y+N279S, W140Y+K280Y, W140Y+G303R, W140Y+E390A, W140Y+G475K, W140Y+A185Q, W140Y+N194F, T182S+A185D, T182S+E189P, T182S+Y242F, T182S+E259G, T182S+F266Y, T182S+N279S, T182S+K280Y, T182S+G303R, T182S+E390A, T182S+G475K, T182S+A185Q, T182S+N194F, A185D+E189P, A185D+Y242F, A185D+E259G, A185D+F266Y, A185D+N279S, A185D+K280Y, A185D+G303R, A185D+E390A, A185D+G475K, A185D, +A185Q, A185D, +N194F, E189P+Y242F, E189P+E259G, E189P+F266Y, E189P+N279S, E189P+K280Y, E189P+G303R, E189P+E390A, E189P+G475K, E189P+A185Q, E189P+N194F, Y242F+E259G, Y242F+F266Y, Y242F+N279S, Y242F+K280Y, Y242F+G303R, Y242F+E390A, Y242F+G475K, Y242F+A185Q, Y242F+N194F, E259G+F266Y, E259G+N279S, E259G+K280Y, E259G+G303R, E259G+E390A, E259G+G475K, E259G+A185Q, E259G+N194F, F266Y+N279S, F266Y+K280Y, F266Y+G303R, F266Y+E390A, F266Y+G475K, F266Y+A185Q, F266Y+N194F, N279+K280Y, N279S+G303R, N279S+E390A, N279S+G475K, N279S+A185Q, N279S+N194F, K280Y+G303R, K280Y+E390A, K280Y+G475K, K280Y+A185Q, K280Y+N194F, G303R+E390A, G303R+G475K, G303R+A185Q, G303R+N194F, E390A+G475K, E390A+A185Q, E390A+N194F, G475K+A185Q, G475K+N194F, A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 14 comprising:

a) deletion at positions corresponding to positions R180* and S181*, using SEQ ID NO: 1 for numbering, and

b) modification at positions corresponding to positions: H1*+D118T+W140Y, H1*+D118T+T182S, H1*+D118T+A185D, H1*+D118T+E189P, H1*+D118T+Y242F, H1*+D118T+E259G, H1*+D118T+F266Y, H1*+D118T+N279S, H1*+D118T+K280Y, H1*+D118T+G303R, H1*+D118T+E390A, H1*+D118T+G475K, H1*+D118T+A185Q, H1*+D118T+N194F, H1*+W140Y+T182S, H1*+W140Y+A185D, H1*+W140Y+E189P, H1*+W140Y+Y242F, H1*+W140Y+E259G, H1*+W140Y+F266Y, H1*+W140Y+N279S, H1*+W140Y+K280Y, H1*+W140Y+G303R, H1*+W140Y+E390A, H1*+W140Y+G475K, H1*+W140Y+A185Q, H1*+W140Y+N194F, H1*+T182S+A185D, H1*+T182S+E189P, H1*+T182S+Y242F, H1*+T182S+E259G, H1*+T182S+F266Y, H1*+T182S+N279S, H1*+T182S+K280Y, H1*+T182S+G303R, H1*+T182S+E390A, H1*+T182S+G475K, H1*+T182S+A185Q, H1*+T182S+N194F, H1*+A185D+E189P, H1*+A185D+Y242F, H1*+A185D+E259G, H1*+A185D+F266Y, H1*+A185D+N279S, H1*+A185D+K280Y, H1*+A185D+G303R, H1*+A185D+E390A, H1*+A185D+G475K, H1*+A185D+A185Q, H1*+A185D+N194F, H1*+E189P+Y242F, H1*+E189P+E259G, H1*+E189P+F266Y, H1*+E189P+N279S, H1*+E189P+K280Y, H1*+E189P+G303R, H1*+E189P+E390A, H1*+E189P+G475K, H1*+E189P+A185Q, H1*+E189P+N194F, H1*+Y242F+E259G, H1*+Y242F+F266Y, H1*+Y242F+N279S, H1*+Y242F+K280Y, H1*+Y242F+G303R, H1*+Y242F+E390A, H1*+Y242F+G475K, H1*+Y242F+A185Q, H1*+Y242F+N194F, H1*+E259G+F266Y, H1*+E259G+N279S, H1*+E259G+K280Y, H1*+E259G+G303R, H1*+E259G+E390A, H1*+E259G+G475K, H1*+E259G+A185Q, H1*+E259G+N194F, H1*+F266Y+N279S, H1*+F266Y+K280Y, H1*+F266Y+G303R, H1*+F266Y+E390A, H1*+F266Y+G475K, H1*+F266Y+A185Q, H1*+F266Y+N194F, H1*+N279S+K280Y, H1*+N279S+G303R, H1*+N279S+E390A, H1*+N279S+G475K, H1*+N279S+A185Q, H1*+N279S+N194F, H1*+K280Y+G303R, H1*+K280Y+E390A, H1*+K280Y+G475K, H1*+K280Y+A185Q, H1*+K280Y+N194F, H1*+G303R+E390A, H1*+G303R+G475K, H1*+G303R+A185Q, H1*+G303R+N194F, H1*+E390A+G475K, H1*+E390A+A185Q, H1*+E390A+N194F, H1*+G475K+A185Q, H1*+G475K+N194F, H1*+A185Q+N194F, D118T+W140Y+T182S, D118T+W140Y+A185D, D118T+W140Y+E189P, D118T+W140Y+Y242F, D118T+W140Y+E259G, D118T+W140Y+F266Y, D118T+W140Y+N279S, D118T+W140Y+K280Y, D118T+W140Y+G303R, D118T+W140Y+E390A, D118T+W140Y+G475K, D118T+W140Y+A185Q, D118T+W140Y+N194F, D118T+T182S+A185D, D118T+T182S+E189P, D118T+T182S+Y242F, D118T+T182S+E259G, D118T+T182S+F266Y, D118T+T182S+N279S, D118T+T182S+K280Y, D118T+T182S+G303R, D118T+T182S+E390A, D118T+T182S+G475K, D118T+T182S+A185Q, D118T+T182S+N194F, D118T+A185D+E189P, D118T+A185D+Y242F, D118T+A185D+E259G, D118T+A185D+F266Y, D118T+A185D+N279S, D118T+A185D+K280Y, D118T+A185D+G303R, D118T+A185D+E390A, D118T+A185D+G475K, D118T+A185D+A185Q, D118T+A185D+N194F, D118T+E189P+Y242F, D118T+E189P+E259G, D118T+E189P+F266Y, D118T+E189P+N279S, D118T+E189P+K280Y, D118T+E189P+G303R, D118T+E189P+E390A, D118T+E189P+G475K, D118T+E189P+A185Q, D118T+E189P+N194F, D118T+Y242F+E259G, D118T+Y242F+F266Y, D118T+Y242F+N279S, D118T+Y242F+K280Y, D118T+Y242F+G303R, D118T+Y242F+E390A, D118T+Y242F+G475K, D118T+Y242F+A185Q, D118T+Y242F+N194F, D118T+E259G+F266Y, D118T+E259G+N279S, D118T+E259G+K280Y, D118T+E259G+G303R, D118T+E259G+E390A, D118T+E259G+G475K, D118T+E259G+A185Q, D118T+E259G+N194F, D118T+F266Y+N279S, D118T+F266Y+K280Y, D118T+F266Y+G303R, D118T+F266Y+E390A, D118T+F266Y+G475K, D118T+F266Y+A185Q, D118T+F266Y+N194F, D118T+N279S+K280Y, D118T+N279S+G303R, D118T+N279S+E390A, D118T+N279S+G475K, D118T+N279S+A185Q, D118T+N279S+N194F, D118T+K280Y+G303R, D118T+K280Y+E390A, D118T+K280Y+G475K, D118T+K280Y+A185Q, D118T+K280Y+N194F, D118T+G303R+E390A, D118T+G303R+G475K, D118T+G303R+A185Q, D118T+G303R+N194F, D118T+E390A+G475K, D118T+E390A+A185Q, D118T+E390A+N194F, D118T+G475K+A185Q, D118T+G475K+N194F, D118T+A185Q+N194F, W140Y+T182S+A185D, W140Y+T182S+E189P, W140Y+T182S+Y242F, W140Y+T182S+E259G, W140Y+T182S+F266Y, W140Y+T182S+N279S, W140Y+T182S+K280Y, W140Y+T182S+G303R, W140Y+T182S+E390A, W140Y+T182S+G475K, W140Y+T182S+A185Q, W140Y+T182S+N194F, W140Y+A185D+E189P, W140Y+A185D+Y242F, W140Y+A185D+E259G, W140Y+A185D+F266Y, W140Y+A185D+N279S, W140Y+A185D+K280Y, W140Y+A185D+G303R, W140Y+A185D+E390A, W140Y+A185D+G475K, W140Y+A185D+A185Q, W140Y+A185D+N194F, W140Y+E189P+Y242F, W140Y+E189P+E259G, W140Y+E189P+F266Y, W140Y+E189P+N279S, W140Y+E189P+K280Y, W140Y+E189P+G303R, W140Y+E189P+E390A, W140Y+E189P+G475K, W140Y+E189P+A185Q, W140Y+E189P+N194F, W140Y+Y242F+E259G, W140Y+Y242F+F266Y, W140Y+Y242F+N279S, W140Y+Y242F+K280Y, W140Y+Y242F+G303R, W140Y+Y242F+E390A, W140Y+Y242F+G475K, W140Y+Y242F+A185Q, W140Y+Y242F+N194F, W140Y+E259G+F266Y, W140Y+E259G+N279S, W140Y+E259G+K280Y, W140Y+E259G+G303R, W140Y+E259G+E390A, W140Y+E259G+G475K, W140Y+E259G+A185Q, W140Y+E259G+N194F, W140Y+F266Y+N279S, W140Y+F266Y+K280Y, W140Y+F266Y+G303R, W140Y+F266Y+E390A, W140Y+F266Y+G475K, W140Y+F266Y+A185Q, W140Y+F266Y+N194F, W140Y+N279S+K280Y, W140Y+N279S+G303R, W140Y+N279S+E390A, W140Y+N279S+G475K, W140Y+N279S+A185Q, W140Y+N279S+N194F, W140Y+K280Y+G303R, W140Y+K280Y+E390A, W140Y+K280Y+G475K, W140Y+K280Y+A185Q, W140Y+K280Y+N194F, W140Y+G303R+E390A, W140Y+G303R+G475K, W140Y+G303R+A185Q, W140Y+G303R+N194F, W140Y+E390A+G475K, W140Y+E390A+A185Q, W140Y+E390A+N194F, W140Y+G475K+A185Q, W140Y+G475K+N194F, W140Y+A185Q+N194F, T182S+A185D+E189P, T182S+A185D+Y242F, T182S+A185D+E259G, T182S+A185D+F266Y, T182S+A185D+N279S, T182S+A185D+K280Y, T182S+A185D+G303R, T182S+A185D+E390A, T182S+A185D+G45K, T182S+A185D+A185Q, T182S+A185D+N194F, T182S+E189P+Y242F, T182S+E189P+E259G, T182S+E189P+F266Y, T182S+E189P+N279S, T182S+E189P+K280Y, T182S+E189P+G303R, T182S+E189P+E390A, T182S+E189P+G475K, T182S+E189P+A185Q, T182S+E189P+N194F, T182S+Y242F+E259G, T182S+Y242F+F266Y, T182S+Y242F+N279S, T182S+Y242F+K280Y, T182S+Y242F+G303R, T182S+Y242F+E390A, T182S+Y242F+G475K, T182S+Y242F+A185Q, T182S+Y242F+N194F, T182S+E259G+F266Y, T182S+E259G+N279S, T182S+E259G+K280Y, T182S+E259G+G303R, T182S+E259G+E390A, T182S+E259G+G475K, T182S+E259G+A185Q, T182S+E259G+N194F, T182S+F266Y+N279S, T182S+F266Y+K280Y, T182S+F266Y+G303R, T182S+F266Y+E390A, T182S+F266Y+G475K, T182S+F266Y+A185Q, T182S+F266Y+N194F, T182S+N279S+K280Y, T182S+N279S+G303R, T182S+N279S+E390A, T182S+N279S+G475K, T182S+N279S+A185Q, T182S+N279S+N194F, T182S+K280Y+G303R, T182S+K280Y+E390A, T182S+K280Y+G475K, T182S+K280Y+A185Q, T182S+K280Y+N194F, T182S+G303R+E390A, T182S+G303R+G475K, T182S+G303R+A185Q, T182S+G303R+N194F, T182S+E390A+G475K, T182S+E390A+A185Q, T182S+E390A+N194F, T182S+G475K+A185Q, T182S+G475K+N194F, T182S+A185Q+N194F, A185D+E189P+Y242F, A185D+E189P+E259G, A185D+E189P+F266Y, A185D+E189P+N279S, A185D+E189P+K280Y, A185D+E189P+G303R, A185D+E189P+E390A, A185D+E189P+G475K, A185D+E189P+A185Q, A185D+E189P+N194F, A185D+Y242F+E259G, A185D+Y242F+F266Y, A185D+Y242F+N279S, A185D+Y242F+K280Y, A185D+Y242F+G303R, A185D+Y242F+E390A, A185D+Y242F+G475K, A185D+Y242F+A185Q, A185D+Y242F+N194F, A185D+E259G+F266Y, A185D+E259G+N279S, A185D+E259G+K280Y, A185D+E259G+G303R, A185D+E259G+E390A, A185D+E259G+G475K, A185D+E259G+A185Q, A185D+E259G+N194F, A185D+F266Y+N279S, A185D+F266Y+K280Y, A185D+F266Y+G303R, A185D+F266Y+E390A, A185D+F266Y+G475K, A185D+F266Y+A185Q, A185D+F266Y+N194F, A185D+N279S+K280Y, A185D+N279S+G303R, A185D+N279S+E390A, A185D+N279S+G475K, A185D+N279S+A185Q, A185D+N279S+N194F, A185D+K280Y+G303R, A185D+K280Y+E390A, A185D+K280Y+G475K, A185D+K280Y+A185Q, A185D+K280Y+N194F, A185D+G303R+E390A, A185D+G303R+G475K, A185D+G303R+A185Q, A185D+G303R+N194F, A185D+E390A+G475K, A185D+E390A+A185Q, A185D+E390A+N194F, A185D+G475K+A185Q, A185D+G475K+N194F, A185D+A185Q+N194F, E189P+Y242F+E259G, E189P+Y242F+F266Y, E189P+Y242F+N279S, E189P+Y242F+K280Y, E189P+Y242F+G303R, E189P+Y242F+E390A, E189P+Y242F+G475K, E189P+Y242F+A185Q, E189P+Y242F+N194F, E189P+E259G+F266Y, E189P+E259G+N279S, E189P+E259G+K280Y, E189P+E259G+G303R, E189P+E259G+E390A, E189P+E259G+G475K, E189P+E259G+A185Q, E189P+E259G+N194F, E189P+F266Y+N279S, E189P+F266Y+K280Y, E189P+F266Y+G303R, E189P+F266Y+E390A, E189P+F266Y+G475K, E189P+F266Y+A185Q, E189P+F266Y+N194F, E189P+N279S+K280Y, E189P+N279S+G303R, E189P+N279S+E390A, E189P+N279S+G475K, E189P+N279S+A185Q, E189P+N279S+N194F, E189P+K280Y+G303R, E189P+K280Y+E390A, E189P+K280Y+G475K, E189P+K280Y+A185Q, E189P+K280Y+N194F, E189P+G303R+E390A, E189P+G303R+G475K, E189P+G303R+A185Q, E189P+G303R+N194F, E189P+E390A+G475K, E189P+E390A+A185Q, E189P+E390A+N194F, E189P+G475K+A185Q, E189P+G475K+N194F, E189P+A185Q+N194F, Y242F+E259G+F266Y, Y242F+E259G+N279S, Y242F+E259G+K280Y, Y242F+E259G+G303R, Y242F+E259G+E390A, Y242F+E259G+G475K, Y242F+E259G+A185Q, Y242F+E259G+N194F, Y242F+F266Y+N279S, Y242F+F266Y+K280Y, Y242F+F266Y+G303R, Y242F+F266Y+E390A, Y242F+F266Y+G475K, Y242F+F266Y+A185Q, Y242F+F266Y+N194F, Y242F+N279S+K280Y, Y242F+N279S+G303R, Y242F+N279S+E390A, Y242F+N279S+G475K, Y242F+N279S+A185Q, Y242F+N279S+N194F, Y242F+K280Y+G303R, Y242F+K280Y+E390A, Y242F+K280Y+G475K, Y242F+K280Y+A185Q, Y242F+K280Y+N194F, Y242F+G303R+E390A, Y242F+G303R+G475K, Y242F+G303R+A185Q, Y242F+G303R+N194F, Y242F+E390A+G475K, Y242F+E390A+A185Q, Y242F+E390A+N194F, Y242F+G475K+A185Q, Y242F+G475K+N194F, Y242F+A185Q+N194F, E259G+F266Y+N279S, E259G+F266Y+K280Y, E259G+F266Y+G303R, E259G+F266Y+E390A, E259G+F266Y+G475K, E259G+F266Y+A185Q, E259G+F266Y+N194F, E259G+N279S+K280Y, E259G+N279S+G303R, E259G+N279S+E390A, E259G+N279S+G475K, E259G+N279S+A185Q, E259G+N279S+N194F, E259G+K280Y+G303R, E259G+K280Y+E390A, E259G+K280Y+G475K, E259G+K280Y+A185Q, E259G+K280Y+N194F, E259G+G303R+E390A, E259G+G303R+G475K, E259G+G303R+A185Q, E259G+G303R+N194F, E259G+E390A+G475K, E259G+E390A+A185Q, E259G+E390A+N194F, E259G+G475K+A185Q, E259G+G475K+N194F, E259G+A185Q+N194F, F266Y+N279S+K280Y, F266Y+N279S+G303R, F266Y+N279S+E390A, F266Y+N279S+G475K, F266Y+N279S+A185Q, F266Y+N279S+N194F, F266Y+K280Y+G303R, F266Y+K280Y+E390A, F266Y+K280Y+G475K, F266Y+K280Y+A185Q, F266Y+K280Y+N194F, F266Y+G303R+E390A, F266Y+G303R+G475K, F266Y+G303R+A185Q, F266Y+G303R+N194F, F266Y+E390A+G475K, F266Y+E390A+A185Q, F266Y+E390A+N194F, F266Y+G475K+A185Q, F266Y+G475K+N194F, F266Y+A185Q+N194F, N279S+K280Y+G303R, N279S+K280Y+E390A, N279S+K280Y+G475K, N279S+K280Y+A185Q, N279S+K280Y+N194F, N279S+G303R+E390A, N279S+G303R+G475K, N279S+G303R+A185Q, N279S+G303R+N194F, N279S+E390A+G475K, N279S+E390A+A185Q, N279S+E390A+N194F, N279S+G475K+A185Q, N279S+G475K+N194F, N279S+A185Q+N194F, K280Y+G303R+E390A, K280Y+G303R+G475K, K280Y+G303R+A185Q, K280Y+G303R+N194F, K280Y+E390A+G475K, K280Y+E390A+A185Q, K280Y+E390A+N194F, K280Y+G475K+A185Q, K280Y+G475K+N194F, K280Y+A185Q+N194F, G303R+E390A+G475K, G303R+E390A+A185Q, G303R+E390A+N194F, G303R+G475K+A185Q, G303R+G475K+N194F, G303R+A185Q+N194F, E390A+G475K+A185Q, E390A+G475K+N194F, E390A+A185Q+N194F, G475K+A185Q+N194F, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 14 comprising:

a) deletion at positions corresponding to positions R180* and S181*, using SEQ ID NO: 1 for numbering, and

b) modification at positions corresponding to positions: H1*+G7A+W140Y+E189P, H1*+G7A+W140Y+Y242F, H1*+G7A+W140Y+E259G, H1*+G7A+W140Y+N279S, H1*+G7A+W140Y+K280Y, H1*+G7A+W140Y+G303R, H1*+G7A+W140Y+E390A, H1*+G7A+E189P+Y242F, H1*+G7A+E189P+E259G, H1*+G7A+E189P+N279S, H1*+G7A+E189P+K280Y, H1*+G7A+E189P+G303R, H1*+G7A+E189P+E390A, H1*+G7A+Y242F+E259G, H1*+G7A+Y242F+N279S, H1*+G7A+Y242F+K280Y, H1*+G7A+Y242F+G303R, H1*+G7A+Y242F+E390A, H1*+G7A+E259G+N279S, H1*+G7A+E259G+K280Y, H1*+G7A+E259G+G303R, H1*+G7A+E259G+E390A, H1*+G7A+N279S+K280Y, H1*+G7A+N279S+G303R, H1*+G7A+N279S+E390A, H1*+G7A+K280Y+G303R, H1*+G7A+K280Y+E390A, H1*+G7A+G303R+E390A, H1*+W140Y+E189P+Y242F, H1*+W140Y+E189P+E259G, H1*+W140Y+E189P+N279S, H1*+W140Y+E189P+K280Y, H1*+W140Y+E189P+G303R, H1*+W140Y+E189P+E390A, H1*+W140Y+Y242F+E259G, H1*+W140Y+Y242F+N279S, H1*+W140Y+Y242F+K280Y, H1*+W140Y+Y242F+G303R, H1*+W140Y+Y242F+E390A, H1*+W140Y+E259G+N279S, H1*+W140Y+E259G+K280Y, H1*+W140Y+E259G+G303R, H1*+W140Y+E259G+E390A, H1*+W140Y+N279S+K280Y, H1*+W140Y+N279S+G303R, H1*+W140Y+N279S+E390A, H1*+W140Y+K280Y+G303R, H1*+W140Y+K280Y+E390A, H1*+W140Y+G303R+E390A, H1*+E189P+Y242F+E259G, H1*+E189P+Y242F+N279S, H1*+E189P+Y242F+K280Y, H1*+E189P+Y242F+G303R, H1*+E189P+Y242F+E390A, H1*+E189P+E259G+N279S, H1*+E189P+E259G+K280Y, H1*+E189P+E259G+G303R, H1*+E189P+E259G+E390A, H1*+E189P+N279S+K280Y, H1*+E189P+N279S+G303R, H1*+E189P+N279S+E390A, H1*+E189P+K280Y+G303R, H1*+E189P+K280Y+E390A, H1*+E189P+G303R+E390A, H1*+Y242F+E259G+N279S, H1*+Y242F+E259G+K280Y, H1*+Y242F+E259G+G303R, H1*+Y242F+E259G+E390A, H1*+Y242F+N279S+K280Y, H1*+Y242F+N279S+G303R, H1*+Y242F+N279S+E390A, H1*+Y242F+K280Y+G303R, H1*+Y242F+K280Y+E390A, H1*+Y242F+G303R+E390A, H1*+E259G+N279S+K280Y, H1*+E259G+N279S+G303R, H1*+E259G+N279S+E390A, H1*+E259G+K280Y+G303R, H1*+E259G+K280Y+E390A, H1*+E259G+G303R+E390A, H1*+N279S+K280Y+G303R, H1*+N279S+K280Y+E390A, H1*+N279S+G303R+E390A, H1*+K280Y+G303R+E390A, G7A+W140Y+E189P+Y242F, G7A+W140Y+E189P+E259G, G7A+W140Y+E189P+N279S, G7A+W140Y+E189P+K280Y, G7A+W140Y+E189P+G303R, G7A+W140Y+E189P+E390A, G7A+W140Y+Y242F+E259G, G7A+W140Y+Y242F+N279S, G7A+W140Y+Y242F+K280Y, G7A+W140Y+Y242F+G303R, G7A+W140Y+Y242F+E390A, G7A+W140Y+E259G+N279S, G7A+W140Y+E259G+K280Y, G7A+W140Y+E259G+G303R, G7A+W140Y+E259G+E390A, G7A+W140Y+N279S+K280Y, G7A+W140Y+N279S+G303R, G7A+W140Y+N279S+E390A, G7A+W140Y+K280Y+G303R, G7A+W140Y+K280Y+E390A, G7A+W140Y+G303R+E390A, G7A+E189P+Y242F+E259G, G7A+E189P+Y242F+N279S, G7A+E189P+Y242F+K280Y, G7A+E189P+Y242F+G303R, G7A+E189P+Y242F+E390A, G7A+E189P+E259G+N279S, G7A+E189P+E259G+K280Y, G7A+E189P+E259G+G303R, G7A+E189P+E259G+E390A, G7A+E189P+N279S+K280Y, G7A+E189P+N279S+G303R, G7A+E189P+N279S+E390A, G7A+E189P+K280Y+G303R, G7A+E189P+K280Y+E390A, G7A+E189P+G303R+E390A, G7A+Y242F+E259G+N279S, G7A+Y242F+E259G+K280Y, G7A+Y242F+E259G+G303R, G7A+Y242F+E259G+E390A, G7A+Y242F+N279S+K280Y, G7A+Y242F+N279S+G303R, G7A+Y242F+N279S+E390A, G7A+Y242F+K280Y+G303R, G7A+Y242F+K280Y+E390A, G7A+Y242F+G303R+E390A, G7A+E259G+N279S+K280Y, G7A+E259G+N279S+G303R, G7A+E259G+N279S+E390A, G7A+E259G+K280Y+G303R, G7A+E259G+K280Y+E390A, G7A+E259G+G303R+E390A, G7A+N279S+K280Y+G303R, G7A+N279S+K280Y+E390A, G7A+N279S+G303R+E390A, G7A+K280Y+G303R+E390A, W140Y+E189P+Y242F+E259G, W140Y+E189P+Y242F+N279S, W140Y+E189P+Y242F+K280Y, W140Y+E189P+Y242F+G303R, W140Y+E189P+Y242F+E390A, W140Y+E189P+E259G+N279S, W140Y+E189P+E259G+K280Y, W140Y+E189P+E259G+G303R, W140Y+E189P+E259G+E390A, W140Y+E189P+N279S+K280Y, W140Y+E189P+N279S+G303R, W140Y+E189P+N279S+E390A, W140Y+E189P+K280Y+G303R, W140Y+E189P+K280Y+E390A, W140Y+E189P+G303R+E390A, W140Y+Y242F+E259G+N279S, W140Y+Y242F+E259G+K280Y, W140Y+Y242F+E259G+G303R, W140Y+Y242F+E259G+E390A, W140Y+Y242F+N279S+K280Y, W140Y+Y242F+N279S+G303R, W140Y+Y242F+N279S+E390A, W140Y+Y242F+K280Y+G303R, W140Y+Y242F+K280Y+E390A, W140Y+Y242F+G303R+E390A, W140Y+E259G+N279S+K280Y, W140Y+E259G+N279S+G303R, W140Y+E259G+N279S+E390A, W140Y+E259G+K280Y+G303R, W140Y+E259G+K280Y+E390A, W140Y+E259G+G303R+E390A, W140Y+N279S+K280Y+G303R, W140Y+N279S+K280Y+E390AW140Y+N279S+G303R+E390A, W140Y+K280Y+G303R+E390A, E189P+Y242F+E259G+N279S, E189P+Y242F+E259G+K280Y, E189P+Y242F+E259G+G303R, E189P+Y242F+E259G+E390A, E189P+Y242F+N279S+K280Y, E189P+Y242F+N279S+G303R, E189P+Y242F+N279S+E390A, E189P+Y242F+K280Y+G303R, E189P+Y242F+K280Y+E390A, E189P+Y242F+G303R+E390A, E189P+E259G+N279S+K280Y, E189P+E259G+N279S+G303R, E189P+E259G+N279S+E390A, E189P+E259G+K280Y+G303R, E189P+E259G+K280Y+E390A, E189P+E259G+G303R+E390A, E189P+N279S+K280Y+G303R, E189P+N279S+K280Y+E390A, E189P+N279S+G303R+E390A, E189P+K280Y+G303R+E390A, Y242F+E259G+N279S+K280Y, Y242F+E259G+N279S+G303R, Y242F+E259G+N279S+E390A, Y242F+E259G+K280Y+G303R, Y242F+E259G+K280Y+E390A, Y242F+E259G+G303R+E390A, Y242F+N279S+K280Y+G303R, Y242F+N279S+K280Y+E390A, Y242F+N279S+G303R+E390A, Y242F+K280Y+G303R+E390A, E259G+N279S+K280Y+G303R, E259G+N279S+K280Y+E390A, E259G+N279S+G303R+E390A, E259G+K280Y+G303R+E390A, N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 14 comprising:

a) deletion at positions corresponding to positions R180* and S181*, using SEQ ID NO: 1 for numbering, and

b) modification at positions corresponding to positions: H1*+G7A+W140Y+E189P+Y242F, H1*+G7A+W140Y+E189P+E259G, H1*+G7A+W140Y+E189P+N279S, H1*+G7A+W140Y+E189P+K280Y, H1*+G7A+W140Y+E189P+G303R, H1*+G7A+W140Y+E189P+E390A, H1*+G7A+W140Y+Y242F+E259G, H1*+G7A+W140Y+Y242F+N279S, H1*+G7A+W140Y+Y242F+K280Y, H1*+G7A+W140Y+Y242F+G303R, H1*+G7A+W140Y+Y242F+E390A, H1*+G7A+W140Y+E259G+N279S, H1*+G7A+W140Y+E259G+K280YH1*+G7A+W140Y+E259G+G303R, H1*+G7A+W140Y+E259G+E390A, H1*+G7A+W140Y+N279S+K280Y, H1*+G7A+W140Y+N279S+G303R, H1*+G7A+W140Y+N279S+E390A, H1*+G7A+W140Y+K280Y+G303R, H1*+G7A+W140Y+K280Y+E390A, H1*+G7A+W140Y+G303R+E390A, H1*+G7A+E189P+Y242F+E259G, H1*+G7A+E189P+Y242F+N279S, H1*+G7A+E189P+Y242F+K280Y, H1*+G7A+E189P+Y242F+G303R, H1*+G7A+E189P+Y242F+E390A, H1*+G7A+E189P+E259G+N279S, H1*+G7A+E189P+E259G+K280Y, H1*+G7A+E189P+E259G+G303R, H1*+G7A+E189P+E259G+E390A, H1*+G7A+E189P+N279S+K280Y, H1*+G7A+E189P+N279S+G303R, H1*+G7A+E189P+N279S+E390A, H1*+G7A+E189P+K280Y+G303R, H1*+G7A+E189P+K280Y+E390A, H1*+G7A+E189P+G303R+E390A, H1*+G7A+Y242F+E259G+N279S, H1*+G7A+Y242F+E259G+K280Y, H1*+G7A+Y242F+E259G+G303R, H1*+G7A+Y242F+E259G+E390A, H1*+G7A+Y242F+N279S+K280Y, H1*+G7A+Y242F+N279S+G303R, H1*+G7A+Y242F+N279S+E390A, H1*+G7A+Y242F+K280Y+G303R, H1*+G7A+Y242F+K280Y+E390A, H1*+G7A+Y242F+G303R+E390A, H1*+G7A+E259G+N279S+K280Y, H1*+G7A+E259G+N279S+G303R, H1*+G7A+E259G+N279S+E390A, H1*+G7A+E259G+K280Y+G303R, H1*+G7A+E259G+K280Y+E390A, H1*+G7A+E259G+G303R+E390A, H1*+G7A+N279S+K280Y+G303R, H1*+G7A+N279S+K280Y+E390A, H1*+G7A+N279S+G303R+E390A, H1*+G7A+K280Y+G303R+E390A, H1*+W140Y+E189P+Y242F+E259G, H1*+W140Y+E189P+Y242F+N279S, H1*+W140Y+E189P+Y242F+K280Y, H1*+W140Y+E189P+Y242F+G303R, H1*+W140Y+E189P+Y242F+E390A, H1*+W140Y+E189P+E259G+N279S, H1*+W140Y+E189P+E259G+K280Y, H1*+W140Y+E189P+E259G+G303R, H1*+W140Y+E189P+E259G+E390A, H1*+W140Y+E189P+N279S+K280Y, H1*+W140Y+E189P+N279S+G303R, H1*+W140Y+E189P+N279S+E390A, H1*+W140Y+E189P+K280Y+G303R, H1*+W140Y+E189P+K280Y+E390A, H1*+W140Y+E189P+G303R+E390A, H1*+W140Y+Y242F+E259G+N279S, H1*+W140Y+Y242F+E259G+K280Y, H1*+W140Y+Y242F+E259G+G303R, H1*+W140Y+Y242F+E259G+E390A, H1*+W140Y+Y242F+N279S+K280Y, H1*+W140Y+Y242F+N279S+G303R, H1*+W140Y+Y242F+N279S+E390A, H1*+W140Y+Y242F+K280Y+G303R, H1*+W140Y+Y242F+K280Y+E390A, H1*+W140Y+Y242F+G303R+E390A, H1*+W140Y+E259G+N279S+K280Y, H1*+W140Y+E259G+N279S+G303R, H1*+W140Y+E259G+N279S+E390A, H1*+W140Y+E259G+K280Y+G303R, H1*+W140Y+E259G+K280Y+E390A, H1*+W140Y+E259G+G303R+E390A, H1*+W140Y+N279S+K280Y+G303R, H1*+W140Y+N279S+K280Y+E390A, H1*+W140Y+N279S+G303R+E390A, H1*+W140Y+K280Y+G303R+E390A, H1*+E189P+Y242F+E259G+N279S, H1*+E189P+Y242F+E259G+K280Y, H1*+E189P+Y242F+E259G+G303R, H1*+E189P+Y242F+E259G+E390A, H1*+E189P+Y242F+N279S+K280Y, H1*+E189P+Y242F+N279S+G303R, H1*+E189P+Y242F+N279S+E390A, H1*+E189P+Y242F+K280Y+G303R, H1*+E189P+Y242F+K280Y+E390A, H1*+E189P+Y242F+G303R+E390A, H1*+E189P+E259G+N279S+K280Y, H1*+E189P+E259G+N279S+G303R, H1*+E189P+E259G+N279S+E390A, H1*+E189P+E259G+K280Y+G303R, H1*+E189P+E259G+K280Y+E390A, H1*+E189P+E259G+G303R+E390A, H1*+E189P+N279S+K280Y+G303R, H1*+E189P+N279S+K280Y+E390A, H1*+E189P+N279S+G303R+E390A, H1*+E189P+K280Y+G303R+E390A, H1*+Y242F+E259G+N279S+K280Y, H1*+Y242F+E259G+N279S+G303R, H1*+Y242F+E259G+N279S+E390A, H1*+Y242F+E259G+K280Y+G303R, H1*+Y242F+E259G+K280Y+E390A, H1*+Y242F+E259G+G303R+E390A, H1*+Y242F+N279S+K280Y+G303R, H1*+Y242F+N279S+K280Y+E390A, H1*+Y242F+N279S+G303R+E390A, H1*+Y242F+K280Y+G303R+E390A, H1*+E259G+N279S+K280Y+G303R, H1*+E259G+N279S+K280Y+E390A, H1*+E259G+N279S+G303R+E390A, H1*+E259G+K280Y+G303R+E390A, H1*+N279S+K280Y+G303R+E390A, G7A+W140Y+E189P+Y242F+E259G, G7A+W140Y+E189P+Y242F+N279S, G7A+W140Y+E189P+Y242F+K280Y, G7A+W140Y+E189P+Y242F+G303R, G7A+W140Y+E189P+Y242F+E390A, G7A+W140Y+E189P+E259G+N279S, G7A+W140Y+E189P+E259G+K280Y, G7A+W140Y+E189P+E259G+G303R, G7A+W140Y+E189P+E259G+E390A, G7A+W140Y+E189P+N279S+K280Y, G7A+W140Y+E189P+N279S+G303R, G7A+W140Y+E189P+N279S+E390A, G7A+W140Y+E189P+K280Y+G303R, G7A+W140Y+E189P+K280Y+E390A, G7A+W140Y+E189P+G303R+E390A, G7A+W140Y+Y242F+E259G+N279S, G7A+W140Y+Y242F+E259G+K280Y, G7A+W140Y+Y242F+E259G+G303R, G7A+W140Y+Y242F+E259G+E390A, G7A+W140Y+Y242F+N279S+K280Y, G7A+W140Y+Y242F+N279S+G303R, G7A+W140Y+Y242F+N279S+E390A, G7A+W140Y+Y242F+K280Y+G303R, G7A+W140Y+Y242F+K280Y+E390A, G7A+W140Y+Y242F+G303R+E390A, G7A+W140Y+E259G+N279S+K280Y, G7A+W140Y+E259G+N279S+G303R, G7A+W140Y+E259G+N279S+E390A, G7A+W140Y+E259G+K280Y+G303R, G7A+W140Y+E259G+K280Y+E390A, G7A+W140Y+E259G+G303R+E390A, G7A+W140Y+N279S+K280Y+G303R, G7A+W140Y+N279S+K280Y+E390A, G7A+W140Y+N279S+G303R+E390A, G7A+W140Y+K280Y+G303R+E390A, G7A+E189P+Y242F+E259G+N279S, G7A+E189P+Y242F+E259G+K280Y, G7A+E189P+Y242F+E259G+G303R, G7A+E189P+Y242F+E259G+E390A, G7A+E189P+Y242F+N279S+K280Y, G7A+E189P+Y242F+N279S+G303R, G7A+E189P+Y242F+N279S+E390A, G7A+E189P+Y242F+K280Y+G303R, G7A+E189P+Y242F+K280Y+E390A, G7A+E189P+Y242F+G303R+E390A, G7A+E189P+E259G+N279S+K280Y, G7A+E189P+E259G+N279S+G303R, G7A+E189P+E259G+N279S+E390A, G7A+E189P+E259G+K280Y+G303R, G7A+E189P+E259G+K280Y+E390A, G7A+E189P+E259G+G303R+E390A, G7A+E189P+N279S+K280Y+G303R, G7A+E189P+N279S+K280Y+E390A, G7A+E189P+N279S+G303R+E390A, G7A+E189P+K280Y+G303R+E390A, G7A+Y242F+E259G+N279S+K280Y, G7A+Y242F+E259G+N279S+G303R, G7A+Y242F+E259G+N279S+E390A, G7A+Y242F+E259G+K280Y+G303R, G7A+Y242F+E259G+K280Y+E390A, G7A+Y242F+E259G+G303R+E390A, G7A+Y242F+N279S+K280Y+G303R, G7A+Y242F+N279S+K280Y+E390A, G7A+Y242F+N279S+G303R+E390A, G7A+Y242F+K280Y+G303R+E390A, G7A+E259G+N279S+K280Y+G303R, G7A+E259G+N279S+K280Y+E390A, G7A+E259G+N279S+G303R+E390A, G7A+E259G+K280Y+G303R+E390A, G7A+N279S+K280Y+G303R+E390A, W140Y+E189P+Y242F+E259G+N279S, W140Y+E189P+Y242F+E259G+K280Y, W140Y+E189P+Y242F+E259G+G303R, W140Y+E189P+Y242F+E259G+E390A, W140Y+E189P+Y242F+N279S+K280Y, W140Y+E189P+Y242F+N279S+G303R, W140Y+E189P+Y242F+N279S+E390A, W140Y+E189P+Y242F+K280Y+G303R, W140Y+E189P+Y242F+K280Y+E390A, W140Y+E189P+Y242F+G303R+E390A, W140Y+E189P+E259G+N279S+K280Y, W140Y+E189P+E259G+N279S+G303R, W140Y+E189P+E259G+N279S+E390A, W140Y+E189P+E259G+K280Y+G303R, W140Y+E189P+E259G+K280Y+E390A, W140Y+E189P+E259G+G303R+E390A, W140Y+E189P+N279S+K280Y+G303R, W140Y+E189P+N279S+K280Y+E390A, W140Y+E189P+N279S+G303R+E390A, W140Y+E189P+K280Y+G303R+E390A, W140Y+Y242F+E259G+N279S+K280Y, W140Y+Y242F+E259G+N279S+G303R, W140Y+Y242F+E259G+N279S+E390A, W140Y+Y242F+E259G+K280Y+G303R, W140Y+Y242F+E259G+K280Y+E390A, W140Y+Y242F+E259G+G303R+E390A, W140Y+Y242F+N279S+K280Y+G303R, W140Y+Y242F+N279S+K280Y+E390A, W140Y+Y242F+N279S+G303R+E390A, W140Y+Y242F+K280Y+G303R+E390A, W140Y+E259G+N279S+K280Y+G303R, W140Y+E259G+N279S+K280Y+E390A, W140Y+E259G+N279S+G303R+E390A, W140Y+E259G+K280Y+G303R+E390A, W140Y+N279S+K280Y+G303R+E390A, E189P+Y242F+E259G+N279S+K280Y, E189P+Y242F+E259G+N279S+G303R, E189P+Y242F+E259G+N279S+E390A, E189P+Y242F+E259G+K280Y+G303R, E189P+Y242F+E259G+K280Y+E390A, E189P+Y242F+E259G+G303R+E390A, E189P+Y242F+N279S+K280Y+G303R, E189P+Y242F+N279S+K280Y+E390A, E189P+Y242F+N279S+G303R+E390A, E189P+Y242F+K280Y+G303R+E390A, E189P+E259G+N279S+K280Y+G303R, E189P+E259G+N279S+K280Y+E390A, E189P+E259G+N279S+G303R+E390A, E189P+E259G+K280Y+G303R+E390A, E189P+N279S+K280Y+G303R+E390A, Y242F+E259G+N279S+K280Y+G303R, Y242F+E259G+N279S+K280Y+E390A, Y242F+E259G+N279S+G303R+E390A, Y242F+E259G+K280Y+G303R+E390A, Y242F+N279S+K280Y+G303R+E390A, E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 14 comprising:

a) deletion at positions corresponding to positions R180* and S181*, using SEQ ID NO: 1 for numbering, and

b) modification at positions corresponding to positions: H1*+G7A+W140Y+E189P+Y242F+E259G, H1*+G7A+W140Y+E189P+Y242F+N279S, H1*+G7A+W140Y+E189P+Y242F+K280Y, H1*+G7A+W140Y+E189P+Y242F+G303R, H1*+G7A+W140Y+E189P+Y242F+E390A, H1*+G7A+W140Y+E189P+E259G+N279S, H1*+G7A+W140Y+E189P+E259G+K280Y, H1*+G7A+W140Y+E189P+E259G+G303R, H1*+G7A+W140Y+E189P+E259G+E390A, H1*+G7A+W140Y+E189P+N279S+K280Y, H1*+G7A+W140Y+E189P+N279S+G303R, H1*+G7A+W140Y+E189P+N279S+E390A, H1*+G7A+W140Y+E189P+K280Y+G303R, H1*+G7A+W140Y+E189P+K280Y+E390A, H1*+G7A+W140Y+E189P+G303R+E390A, H1*+G7A+W140Y+Y242F+E259G+N279S, H1*+G7A+W140Y+Y242F+E259G+K280Y, H1*+G7A+W140Y+Y242F+E259G+G303R, H1*+G7A+W140Y+Y242F+E259G+E390A, H1*+G7A+W140Y+Y242F+N279S+K280Y, H1*+G7A+W140Y+Y242F+N279S+G303R, H1*+G7A+W140Y+Y242F+N279S+E390A, H1*+G7A+W140Y+Y242F+K280Y+G303R, H1*+G7A+W140Y+Y242F+K280Y+E390A, H1*+G7A+W140Y+Y242F+G303R+E390A, H1*+G7A+W140Y+E259G+N279S+K280Y, H1*+G7A+W140Y+E259G+N279S+G303R, H1*+G7A+W140Y+E259G+N279S+E390A, H1*+G7A+W140Y+E259G+K280Y+G303R, H1*+G7A+W140Y+E259G+K280Y+E390A, H1*+G7A+W140Y+E259G+G303R+E390A, H1*+G7A+W140Y+N279S+K280Y+G303R, H1*+G7A+W140Y+N279S+K280Y+E390A, H1*+G7A+W140Y+N279S+G303R+E390A, H1*+G7A+W140Y+K280Y+G303R+E390A, H1*+G7A+E189P+Y242F+E259G+N279S, H1*+G7A+E189P+Y242F+E259G+K280Y, H1*+G7A+E189P+Y242F+E259G+G303R, H1*+G7A+E189P+Y242F+E259G+E390A, H1*+G7A+E189P+Y242F+N279S+K280Y, H1*+G7A+E189P+Y242F+N279S+G303R, H1*+G7A+E189P+Y242F+N279S+E390A, H1*+G7A+E189P+Y242F+K280Y+G303R, H1*+G7A+E189P+Y242F+K280Y+E390A, H1*+G7A+E189P+Y242F+G303R+E390A, H1*+G7A+E189P+E259G+N279S+K280Y, H1*+G7A+E189P+E259G+N279S+G303R, H1*+G7A+E189P+E259G+N279S+E390A, H1*+G7A+E189P+E259G+K280Y+G303R, H1*+G7A+E189P+E259G+K280Y+E390A, H1*+G7A+E189P+E259G+G303R+E390A, H1*+G7A+E189P+N279S+K280Y+G303R, H1*+G7A+E189P+N279S+K280Y+E390A, H1*+G7A+E189P+N279S+G303R+E390A, H1*+G7A+E189P+K280Y+G303R+E390A, H1*+G7A+Y242F+E259G+N279S+K280Y, H1*+G7A+Y242F+E259G+N279S+G303R, H1*+G7A+Y242F+E259G+N279S+E390A, H1*+G7A+Y242F+E259G+K280Y+G303R, H1*+G7A+Y242F+E259G+K280Y+E390A, H1*+G7A+Y242F+E259G+G303R+E390A, H1*+G7A+Y242F+N279S+K280Y+G303R, H1*+G7A+Y242F+N279S+K280Y+E390A, H1*+G7A+Y242F+N279S+G303R+E390A, H1*+G7A+Y242F+K280Y+G303R+E390A, H1*+G7A+E259G+N279S+K280Y+G303R, H1*+G7A+E259G+N279S+K280Y+E390A, H1*+G7A+E259G+N279S+G303R+E390A, H1*+G7A+E259G+K280Y+G303R+E390A, H1*+G7A+N279S+K280Y+G303R+E390A, H1*+W140Y+E189P+Y242F+E259G+N279S, H1*+W140Y+E189P+Y242F+E259G+K280Y, H1*+W140Y+E189P+Y242F+E259G+G303R, H1*+W140Y+E189P+Y242F+E259G+E390A, H1*+W140Y+E189P+Y242F+N279S+K280Y, H1*+W140Y+E189P+Y242F+N279S+G303R, H1*+W140Y+E189P+Y242F+N279S+E390A, H1*+W140Y+E189P+Y242F+K280Y+G303R, H1*+W140Y+E189P+Y242F+K280Y+E390A, H1*+W140Y+E189P+Y242F+G303R+E390A, H1*+W140Y+E189P+E259G+N279S+K280Y, H1*+W140Y+E189P+E259G+N279S+G303R, H1*+W140Y+E189P+E259G+N279S+E390A, H1*+W140Y+E189P+E259G+K280Y+G303R, H1*+W140Y+E189P+E259G+K280Y+E390A, H1*+W140Y+E189P+E259G+G303R+E390A, H1*+W140Y+E189P+N279S+K280Y+G303R, H1*+W140Y+E189P+N279S+K280Y+E390A, H1*+W140Y+E189P+N279S+G303R+E390A, H1*+W140Y+E189P+K280Y+G303R+E390A, H1*+W140Y+Y242F+E259G+N279S+K280Y, H1*+W140Y+Y242F+E259G+N279S+G303R, H1*+W140Y+Y242F+E259G+N279S+E390A, H1*+W140Y+Y242F+E259G+K280Y+G303R, H1*+W140Y+Y242F+E259G+K280Y+E390A, H1*+W140Y+Y242F+E259G+G303R+E390A, H1*+W140Y+Y242F+N279S+K280Y+G303R, H1*+W140Y+Y242F+N279S+K280Y+E390A, H1*+W140Y+Y242F+N279S+G303R+E390A, H1*+W140Y+Y242F+K280Y+G303R+E390A, H1*+W140Y+E259G+N279S+K280Y+G303R, H1*+W140Y+E259G+N279S+K280Y+E390A, H1*+W140Y+E259G+N279S+G303R+E390A, H1*+W140Y+E259G+K280Y+G303R+E390A, H1*+W140Y+N279S+K280Y+G303R+E390A, H1*+E189P+Y242F+E259G+N279S+K280Y, H1*+E189P+Y242F+E259G+N279S+G303R, H1*+E189P+Y242F+E259G+N279S+E390A, H1*+E189P+Y242F+E259G+K280Y+G303R, H1*+E189P+Y242F+E259G+K280Y+E390A, H1*+E189P+Y242F+E259G+G303R+E390A, H1*+E189P+Y242F+N279S+K280Y+G303R, H1*+E189P+Y242F+N279S+K280Y+E390A, H1*+E189P+Y242F+N279S+G303R+E390A, H1*+E189P+Y242F+K280Y+G303R+E390A, H1*+E189P+E259G+N279S+K280Y+G303R, H1*+E189P+E259G+N279S+K280Y+E390A, H1*+E189P+E259G+N279S+G303R+E390A, H1*+E189P+E259G+K280Y+G303R+E390A, H1*+E189P+N279S+K280Y+G303R+E390A, H1*+Y242F+E259G+N279S+K280Y+G303R, H1*+Y242F+E259G+N279S+K280Y+E390A, H1*+Y242F+E259G+N279S+G303R+E390A, H1*+Y242F+E259G+K280Y+G303R+E390A, H1*+Y242F+N279S+K280Y+G303R+E390A, H1*+E259G+N279S+K280Y+G303R+E390A, G7A+W140Y+E189P+Y242F+E259G+N279S, G7A+W140Y+E189P+Y242F+E259G+K280Y, G7A+W140Y+E189P+Y242F+E259G+G303R, G7A+W140Y+E189P+Y242F+E259G+E390A, G7A+W140Y+E189P+Y242F+N279S+K280Y, G7A+W140Y+E189P+Y242F+N279S+G303R, G7A+W140Y+E189P+Y242F+N279S+E390A, G7A+W140Y+E189P+Y242F+K280Y+G303R, G7A+W140Y+E189P+Y242F+K280Y+E390A, G7A+W140Y+E189P+Y242F+G303R+E390A, G7A+W140Y+E189P+E259G+N279S+K280Y, G7A+W140Y+E189P+E259G+N279S+G303R, G7A+W140Y+E189P+E259G+N279S+E390A, G7A+W140Y+E189P+E259G+K280Y+G303R, G7A+W140Y+E189P+E259G+K280Y+E390A, G7A+W140Y+E189P+E259G+G303R+E390A, G7A+W140Y+E189P+N279S+K280Y+G303R, G7A+W140Y+E189P+N279S+K280Y+E390A, G7A+W140Y+E189P+N279S+G303R+E390A, G7A+W140Y+E189P+K280Y+G303R+E390A, G7A+W140Y+Y242F+E259G+N279S+K280Y, G7A+W140Y+Y242F+E259G+N279S+G303R, G7A+W140Y+Y242F+E259G+N279S+E390A, G7A+W140Y+Y242F+E259G+K280Y+G303R, G7A+W140Y+Y242F+E259G+K280Y+E390A, G7A+W140Y+Y242F+E259G+G303R+E390A, G7A+W140Y+Y242F+N279S+K280Y+G303R, G7A+W140Y+Y242F+N279S+K280Y+E390A, G7A+W140Y+Y242F+N279S+G303R+E390A, G7A+W140Y+Y242F+K280Y+G303R+E390A, G7A+W140Y+E259G+N279S+K280Y+G303R, G7A+W140Y+E259G+N279S+K280Y+E390A, G7A+W140Y+E259G+N279S+G303R+E390A, G7A+W140Y+E259G+K280Y+G303R+E390A, G7A+W140Y+N279S+K280Y+G303R+E390A, G7A+E189P+Y242F+E259G+N279S+K280Y, G7A+E189P+Y242F+E259G+N279S+G303R, G7A+E189P+Y242F+E259G+N279S+E390A, G7A+E189P+Y242F+E259G+K280Y+G303R, G7A+E189P+Y242F+E259G+K280Y+E390A, G7A+E189P+Y242F+E259G+G303R+E390A, G7A+E189P+Y242F+N279S+K280Y+G303R, G7A+E189P+Y242F+N279S+K280Y+E390A, G7A+E189P+Y242F+N279S+G303R+E390A, G7A+E189P+Y242F+K280Y+G303R+E390A, G7A+E189P+E259G+N279S+K280Y+G303R, G7A+E189P+E259G+N279S+K280Y+E390A, G7A+E189P+E259G+N279S+G303R+E390A, G7A+E189P+E259G+K280Y+G303R+E390A, G7A+E189P+N279S+K280Y+G303R+E390A, G7A+Y242F+E259G+N279S+K280Y+G303R, G7A+Y242F+E259G+N279S+K280Y+E390A, G7A+Y242F+E259G+N279S+G303R+E390A, G7A+Y242F+E259G+K280Y+G303R+E390A, G7A+Y242F+N279S+K280Y+G303R+E390A, G7A+E259G+N279S+K280Y+G303R+E390A, W140Y+E189P+Y242F+E259G+N279S+K280Y, W140Y+E189P+Y242F+E259G+N279S+G303R, W140Y+E189P+Y242F+E259G+N279S+E390A, W140Y+E189P+Y242F+E259G+K280Y+G303R, W140Y+E189P+Y242F+E259G+K280Y+E390A, W140Y+E189P+Y242F+E259G+G303R+E390A, W140Y+E189P+Y242F+N279S+K280Y+G303R, W140Y+E189P+Y242F+N279S+K280Y+E390A, W140Y+E189P+Y242F+N279S+G303R+E390A, W140Y+E189P+Y242F+K280Y+G303R+E390A, W140Y+E189P+E259G+N279S+K280Y+G303R, W140Y+E189P+E259G+N279S+K280Y+E390A, W140Y+E189P+E259G+N279S+G303R+E390A, W140Y+E189P+E259G+K280Y+G303R+E390A, W140Y+E189P+N279S+K280Y+G303R+E390A, W140Y+Y242F+E259G+N279S+K280Y+G303R, W140Y+Y242F+E259G+N279S+K280Y+E390A, W140Y+Y242F+E259G+N279S+G303R+E390A, W140Y+Y242F+E259G+K280Y+G303R+E390A, W140Y+Y242F+N279S+K280Y+G303R+E390A, W140Y+E259G+N279S+K280Y+G303R+E390A, E189P+Y242F+E259G+N279S+K280Y+G303R, E189P+Y242F+E259G+N279S+K280Y+E390A, E189P+Y242F+E259G+N279S+G303R+E390A, E189P+Y242F+E259G+K280Y+G303R+E390A, E189P+Y242F+N279S+K280Y+G303R+E390A, E189P+E259G+N279S+K280Y+G303R+E390A, Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 14 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S, H1*+G7A+W140Y+E189P+Y242F+E259G+K280Y, H1*+G7A+W140Y+E189P+Y242F+E259G+G303R, H1*+G7A+W140Y+E189P+Y242F+E259G+E390A, H1*+G7A+W140Y+E189P+Y242F+N279S+K280Y, H1*+G7A+W140Y+E189P+Y242F+N279S+G303R, H1*+G7A+W140Y+E189P+Y242F+N279S+E390A, H1*+G7A+W140Y+E189P+Y242F+K280Y+G303R, H1*+G7A+W140Y+E189P+Y242F+K280Y+E390A, H1*+G7A+W140Y+E189P+Y242F+G303R+E390A, H1*+G7A+W140Y+E189P+E259G+N279S+K280Y, H1*+G7A+W140Y+E189P+E259G+N279S+G303R, H1*+G7A+W140Y+E189P+E259G+N279S+E390A, H1*+G7A+W140Y+E189P+E259G+K280Y+G303R, H1*+G7A+W140Y+E189P+E259G+K280Y+E390A, H1*+G7A+W140Y+E189P+E259G+G303R+E390A, H1*+G7A+W140Y+E189P+N279S+K280Y+G303R, H1*+G7A+W140Y+E189P+N279S+K280Y+E390A, H1*+G7A+W140Y+E189P+N279S+G303R+E390A, H1*+G7A+W140Y+E189P+K280Y+G303R+E390A, H1*+G7A+W140Y+Y242F+E259G+N279S+K280Y, H1*+G7A+W140Y+Y242F+E259G+N279S+G303R, H1*+G7A+W140Y+Y242F+E259G+N279S+E390A, H1*+G7A+W140Y+Y242F+E259G+K280Y+G303R, H1*+G7A+W140Y+Y242F+E259G+K280Y+E390A, H1*+G7A+W140Y+Y242F+E259G+G303R+E390A, H1*+G7A+W140Y+Y242F+N279S+K280Y+G303R, H1*+G7A+W140Y+Y242F+N279S+K280Y+E390A, H1*+G7A+W140Y+Y242F+N279S+G303R+E390A, H1*+G7A+W140Y+Y242F+K280Y+G303R+E390A, H1*+G7A+W140Y+E259G+N279S+K280Y+G303R, H1*+G7A+W140Y+E259G+N279S+K280Y+E390A, H1*+G7A+W140Y+E259G+N279S+G303R+E390A, H1*+G7A+W140Y+E259G+K280Y+G303R+E390A, H1*+G7A+W140Y+N279S+K280Y+G303R+E390A, H1*+G7A+E189P+Y242F+E259G+N279S+K280Y, H1*+G7A+E189P+Y242F+E259G+N279S+G303R, H1*+G7A+E189P+Y242F+E259G+N279S+E390A, H1*+G7A+E189P+Y242F+E259G+K280Y+G303R, H1*+G7A+E189P+Y242F+E259G+K280Y+E390A, H1*+G7A+E189P+Y242F+E259G+G303R+E390A, H1*+G7A+E189P+Y242F+N279S+K280Y+G303R, H1*+G7A+E189P+Y242F+N279S+K280Y+E390A, H1*+G7A+E189P+Y242F+N279S+G303R+E390A, H1*+G7A+E189P+Y242F+K280Y+G303R+E390A, H1*+G7A+E189P+E259G+N279S+K280Y+G303R, H1*+G7A+E189P+E259G+N279S+K280Y+E390A, H1*+G7A+E189P+E259G+N279S+G303R+E390A, H1*+G7A+E189P+E259G+K280Y+G303R+E390A, H1*+G7A+E189P+N279S+K280Y+G303R+E390A, H1*+G7A+Y242F+E259G+N279S+K280Y+G303R, H1*+G7A+Y242F+E259G+N279S+K280Y+E390A, H1*+G7A+Y242F+E259G+N279S+G303R+E390A, H1*+G7A+Y242F+E259G+K280Y+G303R+E390A, H1*+G7A+Y242F+N279S+K280Y+G303R+E390A, H1*+G7A+E259G+N279S+K280Y+G303R+E390A, H1*+W140Y+E189P+Y242F+E259G+N279S+K280Y, H1*+W140Y+E189P+Y242F+E259G+N279S+G303R, H1*+W140Y+E189P+Y242F+E259G+N279S+E390A, H1*+W140Y+E189P+Y242F+E259G+K280Y+G303R, H1*+W140Y+E189P+Y242F+E259G+K280Y+E390A, H1*+W140Y+E189P+Y242F+E259G+G303R+E390A, H1*+W140Y+E189P+Y242F+N279S+K280Y+G303R, H1*+W140Y+E189P+Y242F+N279S+K280Y+E390A, H1*+W140Y+E189P+Y242F+N279S+G303R+E390A, H1*+W140Y+E189P+Y242F+K280Y+G303R+E390A, H1*+W140Y+E189P+E259G+N279S+K280Y+G303R, H1*+W140Y+E189P+E259G+N279S+K280Y+E390A, H1*+W140Y+E189P+E259G+N279S+G303R+E390A, H1*+W140Y+E189P+E259G+K280Y+G303R+E390A, H1*+W140Y+E189P+N279S+K280Y+G303R+E390A, H1*+W140Y+Y242F+E259G+N279S+K280Y+G303R, H1*+W140Y+Y242F+E259G+N279S+K280Y+E390A, H1*+W140Y+Y242F+E259G+N279S+G303R+E390A, H1*+W140Y+Y242F+E259G+K280Y+G303R+E390A, H1*+W140Y+Y242F+N279S+K280Y+G303R+E390A, H1*+W140Y+E259G+N279S+K280Y+G303R+E390A, H1*+E189P+Y242F+E259G+N279S+K280Y+G303R, H1*+E189P+Y242F+E259G+N279S+K280Y+E390A, H1*+E189P+Y242F+E259G+N279S+G303R+E390A, H1*+E189P+Y242F+E259G+K280Y+G303R+E390A, H1*+E189P+Y242F+N279S+K280Y+G303R+E390A, H1*+E189P+E259G+N279S+K280Y+G303R+E390A, H1*+Y242F+E259G+N279S+K280Y+G303R+E390A, G7A+W140Y+E189P+Y242F+E259G+N279S+K280Y, G7A+W140Y+E189P+Y242F+E259G+N279S+G303R, G7A+W140Y+E189P+Y242F+E259G+N279S+E390A, G7A+W140Y+E189P+Y242F+E259G+K280Y+G303R, G7A+W140Y+E189P+Y242F+E259G+K280Y+E390A, G7A+W140Y+E189P+Y242F+E259G+G303R+E390A, G7A+W140Y+E189P+Y242F+N279S+K280Y+G303R, G7A+W140Y+E189P+Y242F+N279S+K280Y+E390A, G7A+W140Y+E189P+Y242F+N279S+G303R+E390A, G7A+W140Y+E189P+Y242F+K280Y+G303R+E390A, G7A+W140Y+E189P+E259G+N279S+K280Y+G303R, G7A+W140Y+E189P+E259G+N279S+K280Y+E390A, G7A+W140Y+E189P+E259G+N279S+G303R+E390A, G7A+W140Y+E189P+E259G+K280Y+G303R+E390A, G7A+W140Y+E189P+N279S+K280Y+G303R+E390A, G7A+W140Y+Y242F+E259G+N279S+K280Y+G303R, G7A+W140Y+Y242F+E259G+N279S+K280Y+E390A, G7A+W140Y+Y242F+E259G+N279S+G303R+E390A, G7A+W140Y+Y242F+E259G+K280Y+G303R+E390A, G7A+W140Y+Y242F+N279S+K280Y+G303R+E390A, G7A+W140Y+E259G+N279S+K280Y+G303R+E390A, G7A+E189P+Y242F+E259G+N279S+K280Y+G303R, G7A+E189P+Y242F+E259G+N279S+K280Y+E390A, G7A+E189P+Y242F+E259G+N279S+G303R+E390A, G7A+E189P+Y242F+E259G+K280Y+G303R+E390A, G7A+E189P+Y242F+N279S+K280Y+G303R+E390A, G7A+E189P+E259G+N279S+K280Y+G303R+E390A, G7A+Y242F+E259G+N279S+K280Y+G303R+E390A, W140Y+E189P+Y242F+E259G+N279S+K280Y+G303R, W140Y+E189P+Y242F+E259G+N279S+K280Y+E390A, W140Y+E189P+Y242F+E259G+N279S+G303R+E390A, W140Y+E189P+Y242F+E259G+K280Y+G303R+E390A, W140Y+E189P+Y242F+N279S+K280Y+G303R+E390A, W140Y+E189P+E259G+N279S+K280Y+G303R+E390A, W140Y+Y242F+E259G+N279S+K280Y+G303R+E390A, E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 14 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y, H1*+G7A+W140Y+E189P+Y242F+E259G+N279S+G303R, H1*+G7A+W140Y+E189P+Y242F+E259G+N279S+E390A, H1*+G7A+W140Y+E189P+Y242F+E259G+K280Y+G303R, H1*+G7A+W140Y+E189P+Y242F+E259G+K280Y+E390A, H1*+G7A+W140Y+E189P+Y242F+E259G+G303R+E390A, H1*+G7A+W140Y+E189P+Y242F+N279S+K280Y+G303R, H1*+G7A+W140Y+E189P+Y242F+N279S+K280Y+E390A, H1*+G7A+W140Y+E189P+Y242F+N279S+G303R+E390A, H1*+G7A+W140Y+E189P+Y242F+K280Y+G303R+E390A, H1*+G7A+W140Y+E189P+E259G+N279S+K280Y+G303R, H1*+G7A+W140Y+E189P+E259G+N279S+K280Y+E390A, H1*+G7A+W140Y+E189P+E259G+N279S+G303R+E390A, H1*+G7A+W140Y+E189P+E259G+K280Y+G303R+E390A, H1*+G7A+W140Y+E189P+N279S+K280Y+G303R+E390A, H1*+G7A+W140Y+Y242F+E259G+N279S+K280Y+G303R, H1*+G7A+W140Y+Y242F+E259G+N279S+K280Y+E390A, H1*+G7A+W140Y+Y242F+E259G+N279S+G303R+E390A, H1*+G7A+W140Y+Y242F+E259G+K280Y+G303R+E390A, H1*+G7A+W140Y+Y242F+N279S+K280Y+G303R+E390A, H1*+G7A+W140Y+E259G+N279S+K280Y+G303R+E390A, H1*+G7A+E189P+Y242F+E259G+N279S+K280Y+G303R, H1*+G7A+E189P+Y242F+E259G+N279S+K280Y+E390A, H1*+G7A+E189P+Y242F+E259G+N279S+G303R+E390A, H1*+G7A+E189P+Y242F+E259G+K280Y+G303R+E390A, H1*+G7A+E189P+Y242F+N279S+K280Y+G303R+E390A, H1*+G7A+E189P+E259G+N279S+K280Y+G303R+E390A, H1*+G7A+Y242F+E259G+N279S+K280Y+G303R+E390A, H1*+W140Y+E189P+Y242F+E259G+N279S+K280Y+G303R, H1*+W140Y+E189P+Y242F+E259G+N279S+K280Y+E390A, H1*+W140Y+E189P+Y242F+E259G+N279S+G303R+E390A, H1*+W140Y+E189P+Y242F+E259G+K280Y+G303R+E390A, H1*+W140Y+E189P+Y242F+N279S+K280Y+G303R+E390A, H1*+W140Y+E189P+E259G+N279S+K280Y+G303R+E390A, H1*+W140Y+Y242F+E259G+N279S+K280Y+G303R+E390A, H1*+E189P+Y242F+E259G+N279S+K280Y+G303R+E390A, G7A+W140Y+E189P+Y242F+E259G+N279S+K280Y+G303R, G7A+W140Y+E189P+Y242F+E259G+N279S+K280Y+E390A, G7A+W140Y+E189P+Y242F+E259G+N279S+G303R+E390A, G7A+W140Y+E189P+Y242F+E259G+K280Y+G303R+E390A, G7A+W140Y+E189P+Y242F+N279S+K280Y+G303R+E390A, G7A+W140Y+E189P+E259G+N279S+K280Y+G303R+E390A, G7A+W140Y+Y242F+E259G+N279S+K280Y+G303R+E390A, G7A+E189P+Y242F+E259G+N279S+K280Y+G303R+E390A, W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 14 comprising:

a) deletion at positions corresponding to positions R180* and S181*, using SEQ ID NO: 1 for numbering, and

b) modification at positions corresponding to positions: H1*+G7A+W140Y+E189P+Y242F+E259G+N279S+K280Y+G303R, H1*+G7A+W140Y+E189P+Y242F+E259G+N279S+K280Y+E390A, H1*+G7A+W140Y+E189P+Y242F+E259G+N279S+G303R+E390A, H1*+G7A+W140Y+E189P+Y242F+E259G+K280Y+G303R+E390A, H1*+G7A+W140Y+E189P+Y242F+N279S+K280Y+G303R+E390A, H1*+G7A+W140Y+E189P+E259G+N279S+K280Y+G303R+E390A, H1*+G7A+W140Y+Y242F+E259G+N279S+K280Y+G303R+E390A, H1*+G7A+E189P+Y242F+E259G+N279S+K280Y+G303R+E390A, H1*+W140Y+E189P+Y242F+E259G+N279S+K280Y+G303R+E390A, G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 14 comprising:

a) deletion at positions corresponding to positions R180* and S181*, using SEQ ID NO: 1 for numbering, and

H1*+G7A+W140Y+E189P+Y242F+E259G+N279S+K280Y+G303R+E390A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 1 comprising modifications in the positions corresponding to: H1*+G7A+R180*+S181*, H1*+G7A+R180*+S181*+Y242F, H1*+G7A+R180*+S181*+Y242F+N279S, H1*+G7A+R180*+S181*+Y242F+N279S+G303R, H1*+G7A+R180*+S181*+Y242F+E259G+N279S+G303R, H1*+G7A+R180*+S181*+Y242F+E259G+N279S+G303R+E390A, H1*+G7A+R180*+S181*+Y242F+E259G+N279S+G303R+E390A+G475K, H1*+G7A+R180*+S181*+Y242F+E259G+N279S+E390A+G476A, H1*+G7A+R180*+S181*+Y242F+E259G+N279S+G303R+E390A+G476A, H1*+G7A+R180*+S181*+D118T+Y242F+E259G+N279S+G303R+E390A+G475K, H1*+G7A+R180*+S181*+D118T+W140Y+Y242F+E259G+N279S+E390A+G476A, H1*+G7A+R180*+S181*+D118T+W140Y+Y242F+E259G+N279S+G303R+E390A+G476A, H1*+G7A+R180*+S181*+D118T+W140Y+Y242F+E259G+N279S+G303R+E390A+G475K, H1*+G7A+R180*+S181*+D118T+W140Y+Y242F+E259G+N279S+E390A+G476A, H1*+G7A+R180*+S181*+D118T+W140Y+Y242F+E259G+N279S+G303R+E390A+G476A, H1*+G7A+R180*+S181*+D118T+W140Y+N194F+Y242F+E259G+N279S+G303R+E390A+G475K, H1*+G7A+R180*+S181*+D118T+W140Y+N194F+Y242F+E259G+N279S+E390A+G476A, H1*+G7A+R180*+S181*+D118T+W140Y+N194F+Y242F+E259G+N279S+G303R+E390A+G476A, H1*+G7A+R180*+S181*+D118T+W140Y+N194F+Y242F+E259G+K280Y+N279S+G303R+E390A+G475K, H1*+G7A+R180*+S181*+D118T+W140Y+N194F+Y242F+E259G+K280Y+N279S+E390A+G476A, H1*+G7A+R180*+S181*+D118T+W140Y+N194F+Y242F+E259G+K280Y+N279S+G303R+E390A+G476A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

In one aspect, the alpha-amylase variant of SEQ ID NO: 1 comprising modifications in the positions corresponding to E169I+R180*+S181*, T114K+R180*+S181*, W167A+R180*+S181*, E169V+R180*+S181*, R180*+S181*+W188C, W167D+R180*+S181*, Y177D+R180*+S181*, W167S+R180*+S181*, W167E+R180*+S181*, W167P+R180*+S181*, R180*+S181*+L205R, R180*+S181*+A203F, W159L+R180*+S181*, Y160P+R180*+S181*, W159H+R180*+S181*, K178A+R180*+S181*, R180*+S181*+G183P, Q136T+R180*+S181*, K142P+R180*+S181*, K142E+R180*+S181*, Y152L+R180*+S181*, A111L+R180*+S181*, G110K+R180*+S181*, A111G+R180*+S181* and V117G+R180*+S181*, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to R180*+S181*+E193+H323N, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to G7H+Q98L+G110K+R180*+S181*, G7H+W167P+R180*+S181*+G455R and Q98L+V103A+G110K+R180*+S181*, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to A60V+Q98L+G110K+R180*+S181*+A383D, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 1 comprising modifications in the positions corresponding to F116L+D123N+N126D+T131I+R180*+S181*+F202Y and Q98L+G110K+S132D+Y135E+R180*+S181*+T315A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to H1*+G7A+R180*+S181*+Y242F+E259G+N279S+G303R+E390A+G475K, H1*+G7A+S181*+T182*+Y242F+E259G+N279S+G303R+E390A+G475K and H1*+G7S+R180*+S181*+Y242F+E259G+N279S+G303R+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to H1*+G7A+R180*+S181*+T182S+Y242F+E259G+N279S+G303R+E390A+G475K, H1*+G7A+R180*+T182L+G183*+Y242F+E259G+N279S+G303R+E390A+G475K, H1*+G7A+S181L+T182*+G183*+Y242F+E259G+N279S+G303R+E390A+G475K, H1*+G7A+R180S+T182*+G183*+Y242F+E259G+N279S+G303R+E390A+G475K, H1*+G7A+A60P+R180*+S181*+Y242F+E259G+N279S+G303R+E390A+G475K, H1*+G7A+M105L+R180*+S181*+Y242F+E259G+N279S+G303R+E390A+G475K, H1*+G7A+M105I+R180*+S181*+Y242F+E259G+N279S+G303R+E390A+G475K, H1*+G7A+R180*+S181*+T182D+Y242F+E259G+N279S+G303R+E390A+G475K, H1*+G7A+R180*+S181*+T182E+Y242F+E259G+N279S+G303R+E390A+G475K, H1*+G7A+R180*+S181*+T182G+Y242F+E259G+N279S+G303R+E390A+G475K, H1*+G7A+R180*+S181*+T182M+Y242F+E259G+N279S+G303R+E390A+G475K, H1*+G7A+R180*+S181*+A185K+Y242F+E259G+N279S+G303R+E390A+G475K, H1*+G7A+R180*+S181*+A185D+Y242F+E259G+N279S+G303R+E390A+G475K, H1*+G7A+R180*+S181*+A185Q+Y242F+E259G+N279S+G303R+E390A+G475K, H1*+G7A+R180*+S181*+A185N+Y242F+E259G+N279S+G303R+E390A+G475K, H1*+G7A+R180*+S181*+A185R+Y242F+E259G+N279S+G303R+E390A+G475K, H1*+G7A+R180*+S181*+A185E+Y242F+E259G+N279S+G303R+E390A+G475K, H1*+G7A+R180*+S181*+E189P+Y242F+E259G+N279S+G303R+E390A+G475K, H1*+G7A+R180*+S181*+L205Y+Y242F+E259G+N279S+G303R+E390A+G475K, H1*+G7A+R180*+S181*+L205F+Y242F+E259G+N279S+G303R+E390A+G475K, H1*+G7A+R180*+S181*+Y242F+S243Q+E259G+N279S+G303R+E390A+G475K, H1*+G7A+R180*+S181*+Y242F+F244L+E259G+N279S+G303R+E390A+G475K, H1*+G7A+R180*+S181*+Y242F+F244I+E259G+N279S+G303R+E390A+G475K, H1*+G7A+R180*+S181*+Y242F+E259G+F266Y+N279S+G303R+E390A+G475K, H1*+G7A+R180*+S181*+N194F+Y242F+E259G+N279S+G303R+E390A+G475K, H1*+G7A+R180*+S181*+N196F+Y242F+E259G+N279S+G303R+E390A+G475K, H1*+G7A+R180*+S181*+Y242F+E259G+N279S+G303R+M322G+E390A+G475K, H1*+G7A+T40N+R180*+S181*+Y242F+E259G+N279S+G303R+E390A+G475K, H1*+G7S+Q98L+R180*+S181*+Y242F+E259G+N279S+G303R+E390A+G475K, H1*+G7S+R180*+S181*+Y242F+E259G+F261K+N279S+G303R+E390A+G475K, H1*+G7S+R180*+S181*+Y242F+E259G+N279S+G303R+Y362V+E390A+G475K, H1*+G7A+R180*+S181*+Y242F+E259G+N279S+G303R+Y362V+E390A+G475K, H1*+G7S+R180*+S181*+Y242F+E259G+F261S+N279S+G303R+E390A+G475K, H1*+G7A+R180*+S181*+Y242F+E259G+N279S+S286L+G303R+E390A+G475K, H1*+G7A+G110K+R180*+S181*+Y242F+E259G+N279S+G303R+E390A+G475K, H1*+G7A+W140S+R180*+S181*+Y242F+E259G+N279S+G303R+E390A+G475K, H1*+G7A+W167P+R180*+S181*+Y242F+E259G+N279S+G303R+E390A+G475K, H1*+G7A+R180*+S181*+Y242F+E259G+N279S+G303R+H323A+E390A+G475K, H1*+G7A+R180*+S181*+Y242F+E259G+N279S+G303R+H320T+E390A+G475K, H1*+G7A+R180*+S181*+Y242F+E259G+N279S+G303R+P321H+E390A+G475K, H1*+G7A+R180*+S181*+Y242F+E259G+N279S+G303R+M322Y+E390A+G475K, H1*+G7A+R180*+S181*+Y242F+E259G+N279S+G303R+Q360Y+E390A+G475K, H1*+G7A+Q98L+R180*+S181*+Y242F+E259G+N279S+G303R+E390A+G475K, H1*+G7A+R180*+S181*+Y242F+E259G+F261S+N279S+G303R+E390A+G475K, H1*+G7H+G110K+R180*+S181*+Y242F+E259G+N279S+G303R+E390A+G475K, H1*+G7A+Q98L+R180*+S181*+Y242F+E259G+N279S+G303R+E390A+G475K, H1*+G7A+R180*+S181*+Y242F+E259G+F261K+N279S+G303R+E390A+G475K and L63F+W140Y+L173F+R180*+S181*+N194F+L205Y+Y242F+E259G+N279S+H323W, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to H1*+G7A+R180*+S181*+N194F+L205Y+Y242F+E259G+N279S+G303R+E390A+G475K, H1*+G7A+R180*+S181*+N194F+L205F+Y242F+E259G+N279S+G303R+E390A+G475K, H1*+G7A+R180*+S181*+N196F+L205Y+Y242F+E259G+N279S+G303R+E390A+G475K, H1*+G7A+R180*+S181*+E189P+Y242F+E259G+N276F+N279S+G303R+E390A+G475K, H1*+G7A+R180*+S181*+E189P+Y242F+E259G+N276Y+N279S+G303R+E390A+G475K, H1*+G7A+R180*+S181*+E189P+Y242F+E259G+N276W+N279S+G303R+E390A+G475K, H1*+G7A+R180*+S181*+E189P+Y242F+E259G+N279S+K280Y+G303R+E390A+G475K, H1*+G7A+R180*+S181*+E189P+Y242F+E259G+N279S+K280W+G303R+E390A+G475K, H1*+G7A+R180*+S181*+Y242F+E259G+N279S+G303R+M322G+Q360W+E390A+G475K, H1*+G7A+R180*+S181*+Y242F+E259G+N279S+G303R+E359W+Q360W+E390A+G475K, H1*+G7S+R180*+S181*+Y242F+E259G+F261S+N279S+G303R+Y362V+E390A+G475K, H1*+G7A+T40N+Q98L+R180*+S181*+Y242F+E259G+N279S+G303R+E390A+G475K, H1*+G7S+T40N+Q98L+R180*+S181*+Y242F+E259G+N279S+G303R+E390A+G475K, H1*+G7A+D123N+I176V+R180*+S181*+Y242F+E259G+N279S+G303R+E390A+G475K, H1*+G7A+R180*+S181*+Y242F+E259G+N279S+G303R+M322G+Q360W+E390A+G475K, H1*+G7A+T40N+Q98L+R180*+S181*+Y242F+E259G+N279S+G303R+E390A+G475K, H1*+G7A+R180*+S181*+Y242F+E259G+N279S+S286K+G303R+H323A+E390A+G475K, H1*+G7A+R180*+S181*+Y242F+E259G+N279S+G303R+H320T+H323A+E390A+G475K, H1*+G7A+R180*+S181*+Y242F+E259G+N279S+G303R+P321H+M322Y+E390A+G475K, H1*+G7A+R180*+S181*+Y242F+E259G+N279S+G303R+P321H+Q360Y+E390A+G475K, H1*+G7A+R180*+S181*+Y242F+E259G+N279S+G303R+M322Y+Q360Y+E390A+G475K, H1*+G7S+T40N+R180*+S181*+Y242F+E259G+N279S+G303R+Y362V+E390A+G475K, H1*+G7S+R180*+S181*+Y242F+E259G+F261S+N279S+G303R+Y362V+E390A+G475K, H1*+G7S+T40N+Q98L+R180*+S181*+Y242F+E259G+N279S+G303R+E390A+G475K, H1*+G7A+R180*+S181*+Y242F+E259G+F261S+N279S+G303R+Y362V+E390A+G475K, H1*+G7A+R180*+S181*+Y242F+E259G+N279S+S286L+G303R+H320T+E390A+G475K, H1*+G7H+Q98L+G110K+R180*+S181*+Y242F+E259G+N279S+G303R+E390A+G475K, H1*+G7A+Q98L+G110K+R180*+S181*+Y242F+E259G+N279S+G303R+E390A+G475K, H1*+G7H+Q98L+G110K+R180*+S181*+Y242F+E259G+N279S+G303R+E390A+G475K, H1*+G7A+Q98L+G110K+R180*+S181*+Y242F+E259G+N279S+G303R+E390A+G475K, H1*+G7A+R180*+S181*+Y242F+E259G+N279S+S286L+G303R+Y362V+E390A+G475K, H1*+G7A+R180*+S181*+Y242F+E259G+N279S+W283N+S286K+G303R+E390A+G475K, H1*+G7A+R180*+S181*+Y242F+E259G+N279S+W283N+G303R+H320T+E390A+G475K, H1*+G7A+R180*+S181*+Y242F+E259G+N279S+W283N+G303R+H323A+E390A+G475K, H1*+G7A+R180*+S181*+Y242F+E259G+N279S+S286K+G303R+H320T+E390A+G475K, H1*+G7A+R180*+S181*+Y242F+E259G+N279S+S286K+G303R+Y362V+E390A+G475K, H1*+G7A+R180*+S181*+Y242F+E259G+N279S+G303R+M322G+E359W+E390A+G475K and W140Y+L173F+R180*+S181*+N194F+L205Y+N225S+Y242F+E259G+N279S+G303R+H323A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to H1*+G7A+R180*+S181*+E189P+L205F+Y242F+E259G+N276F+N279S+G303R+E390A+G475K, H1*+G7A+R180*+S181*+E189P+L205F+Y242F+E259G+N276Y+N279S+G303R+E390A+G475K, H1*+G7A+R180*+S181*+E189P+L205F+Y242F+E259G+N276W+N279S+G303R+E390A+G475K, H1*+G7A+R180*+S181*+E189P+L205F+Y242F+E259G+N279S+K280F+G303R+E390A+G475K, H1*+G7A+R180*+S181*+E189P+L205F+Y242F+E259G+N279S+K280Y+G303R+E390A+G475K, H1*+G7A+R180*+S181*+E189P+L205F+Y242F+E259G+N279S+K280W+G303R+E390A+G475K, H1*+G7A+R180*+S181*+E189P+L205Y+Y242F+E259G+N276F+N279S+G303R+E390A+G475K, H1*+G7A+R180*+S181*+E189P+L205Y+Y242F+E259G+N276Y+N279S+G303R+E390A+G475K, H1*+G7A+R180*+S181*+E189P+L205Y+Y242F+E259G+N276W+N279S+G303R+E390A+G475K, H1*+G7A+R180*+S181*+E189P+L205Y+Y242F+E259G+N279S+K280F+G303R+E390A+G475K, H1*+G7A+R180*+S181*+E189P+L205Y+Y242F+E259G+N279S+K280Y+G303R+E390A+G475K, H1*+G7A+R180*+S181*+E189P+L205Y+Y242F+E259G+N279S+K280W+G303R+E390A+G475K, H1*+G7A+G113S+F116W+D123N+S181*+T182*+Y242F+E259G+N279S+G303R+E390A+G475K, H1*+G7S+T40N+Q98L+R180*+S181*+Y242F+E259G+N279S+G303R+Y362V+E390A+G475K, H1*+G7A+R180*+S181*+Y242F+E259G+N279S+G303R+M322G+E359W+Q360W+E390A+G475K, H1*+G7A+R180*+S181*+Y242F+E259G+F261S+N279S+S286K+G303R+Y362V+E390A+G475K, H1*+G7A+R180*+S181*+Y242F+E259G+N279S+G303R+P321H+M322Y+Q360Y+E390A+G475K, H1*+G7A+G113S+F116W+D123N+R180*+S181*+Y242F+E259G+N279S+G303R+E390A+G475K, H1*+G7S+T40N+Q98L+R180*+S181*+Y242F+E259G+N279S+G303R+H320T+E390A+G475K, H1*+G7S+T40N+Q98L+R180*+S181*+Y242F+E259G+N279S+G303R+Y362V+E390A+G475K, H1*+G7A+1137F+R180*+S181*+Y242F+E259G+F261S+N279S+G303R+Y362V+E390A+G475K, H1*+G7A+R180*+S181*+Y242F+E259G+F261S+N279S+S286K+G303R+Y362V+E390A+G475K, H1*+G7A+R180*+S181*+Y242F+E259G+N279S+W283N+G303R+H320T+H323A+E390A+G475K, H1*+G7A+Q98L+R180*+S181*+Y242F+E259G+F261K+N279S+G303R+Y362V+E390A+G475K, H1*+G7H+Q98L+R180*+S181*+Y242F+E259G+N279S+G303R+A383D+E390A+D405G+G475K, H1*+G7A+Q98L+G110K+R180*+S181*+Y242F+T256A+E259G+N279S+G303R+E390A+G475K, H1*+G7A+Q98L+G110K+R180*+S181*+Y242F+E259G+N279S+G303R+A383D+E390A+G475K, H1*+G7A+W140Y+R180*+S181*+N194F+L205Y+Y242F+E259G+N279S+G303R+E390A+G476A, H1*+G7A+W140Y+R180*+S181*+N194F+L205Y+Y242F+E259G+N279S+G303R+N394W+G475K, H1*+G7A+W140Y+R180*+S181*+N194F+L205Y+Y242F+E259G+N279S+G303R+E390A+N474L, H1*+G7A+W140Y+R180*+S181*+N194F+L205Y+Y242F+E259G+N279S+S302T+E390A+G475K, H1*+G7A+W140Y+R180*+S181*+N194F+L205Y+Y242F+E259G+N279S+G303R+E390A+G475E, H1*+G7A+W140Y+R180*+S181*+N194F+L205Y+Y242F+E259G+N279S+G303R+E390A+N474F, H1*+G7A+W140Y+R180*+S181*+N194F+L205Y+Y242F+E259G+N279S+S302Y+E390A+G475K, H1*+G7A+W140Y+R180*+S181*+N194F+L205Y+Y242F+T256D+N279S+G303R+E390A+G475K, H1*+G7A+R37A+W140Y+R180*+S181*+N194F+L205Y+Y242F+E259G+N279S+G303R+G475K, H1*+W140Y+R180*+S181*+N194F+L205Y+Y242F+E259G+N279S+G303R+Q360Y+E390A+G475K, H1*+G7A+R37N+W140Y+R180*+S181*+N194F+L205Y+Y242F+E259G+N279S+G303R+G475K, H1*+W140Y+R180*+S181*+N194F+L205Y+Y242F+E259G+N279S+G303R+E390A+R414M+G475K, H1*+W140Y+R180*+S181*+N194F+L205Y+Y242F+E259G+F261P+N279S+G303R+E390A+G475K, H1*+W140Y+R180*+S181*+N194F+L205Y+Y242F+E259G+N279S+G303R+H323W+E390A+G475K, H1*+W140Y+R180*+S181*+N194F+L205Y+Y242F+E259G+N279S+G303R+P321Y+E390A+G475K, H1*+W140Y+R180*+S181*+N194F+L205Y+Y242F+E259G+N279S+G303R+Q360M+E390A+G475K, H1*+G7A+W140Y+R180*+S181*+Y242F+S243Q+E259G+N279S+K280Y+G303R+E390A+G475K, H1*+G7A+W140Y+R180*+S181*+Y242F+E259G+F266Y+N279S+K280Y+G303R+E390A+G475K and H1*+G7A+W140Y+R180*+S181*+T182D+A185Q+Y242F+E259G+N279S+G303R+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding: H1*+G7A+R180*+S181*+Y242F+E259G+N279S+W283N+S286K+G303R+H320T+H323A+E390A+G475K, H1*+G7A+W140Y+R180*+S181*+T182D+N194F+L205Y+Y242F+E259G+N279S+G303R+E390A+G475K, H1*+G7A+W140Y+R180*+S181*+T182D+N194F+L205V+Y242F+E259G+N279S+G303R+E390A+G475K, H1*+G7A+W140Y+R180*+S181*+T182S+N194F+L205Y+Y242F+E259G+N279S+G303R+E390A+G475K, H1*+G7A+W140Y+R180*+S181*+T182S+N194F+L205V+Y242F+E259G+N279S+G303R+E390A+G475K, H1*+G7A+D123N+W140Y+R180*+S181*+N194F+L205V+Y242F+E259G+N279S+G303R+E390A+G475K, H1*+G7A+D123N+W140Y+R180*+S181*+N194F+L205Y+Y242F+E259G+N279S+G303R+E390A+G475K, H1*+G7A+Q98L+G110K+P146S+R180*+S181*+Y242F+E259G+N279S+G303R+A383D+E390A+G475K, H1*+N3S+G7A+Q98L+G110K+R180*+S181*+Y242F+V252+E259G+N279S+G303R+E390A+G475K, H1*+G7A+W140Y+R180*+S181*+N194F+L205V+Y242F+E259G+F266Y+N279S+G303R+E390A+G475K, H1*+G7A+W140Y+R180*+S181*+A185Q+N194F+L205V+Y242F+E259G+N279S+G303R+E390A+G475K, H1*+G7A+W140Y+R180*+S181*+A185Q+N194F+L205Y+Y242F+E259G+N279S+G303R+E390A+G475K, H1*+G7A+W140Y+R180*+S181*+E189P+N194F+Y242F+E259G+N279S+K280Y+G303R+E390A+G475K, H1*+G7A+W140Y+R180*+S181*+E189P+N194F+Y242F+E259G+N279S+K280Y+G303R+E390A+G475K, H1*+G7A+W140Y+R180*+S181*+N194F+L205Y+Y242F+E259G+F266Y+N279S+G303R+E390A+G475K, H1*+G7A+W140Y+R180*+S181*+N194F+L205Y+Y242F+E259G+N279S+S302T+G303R+E390A+G475K, H1*+G7A+W140Y+R180*+S181*+N194F+L205Y+Y242F+E259G+N279S+G303R+E390A+N474F+G475K, H1*+G7A+W140Y+R180*+S181*+N194F+L205Y+Y242F+E259G+N279S+G303R+E390A+N422V+G475K, H1*+G7A+W140Y+R180*+S181*+N194F+L205Y+Y242F+E259G+F261P+N279S+G303R+E390A+G475K, H1*+G7A+W140Y+R180*+S181*+N194F+L205Y+Y242F+E259G+N279S+G303R+E390A+N470A+G475K, H1*+G7A+W140Y+R180*+S181*+N194F+L205Y+Y242L+E259G+N279S+G303R+W346N+E390A+G475K, H1*+G7A+W140Y+R180*+S181*+N194F+L205Y+Y242F+E259G+N279S+G303R+E390A+H420P+G475K, H1*+G7A+R26Y+W140Y+R180*+S181*+N194F+L205Y+Y242F+E259G+N279S+G303R+E390A+G475K, H1*+G7A+W140Y+R180*+S181*+N194F+L205Y+Y242F+E259G+N279S+G303R+Y371S+E390A+G475K, H1*+G7A+W140Y+R180*+S181*+T182D+A185Q+Y242F+E259G+N279S+K280Y+G303R+E390A+G475K, H1*+G7A+W140Y+R180*+S181*+A185Q+E189P+Y242F+E259G+N279S+K280Y+G303R+E390A+G475K, H1*+G7A+W140Y+R180*+S181*+E189P+Y242F+S243Q+E259G+N279S+K280Y+G303R+E390A+G475K, H1*+G7A+W140Y+R180*+S181*+T182D+Y242F+S243Q+E259G+N279S+K280Y+G303R+E390A+G475K, H1*+G7A+W140Y+R180*+S181*+Y242F+S243Q+E259G+F266Y+N279S+K280Y+G303R+E390A+G475K, H1*+G7A+A41D+W140Y+R180*+S181*+N194F+L205Y+Y242F+E259G+N279S+G303R+E390A+G475K and H1*+G7A+T40G+W140Y+R180*+S181*+N194F+L205Y+Y242F+E259G+N279S+G303R+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding: H1*+G7A+F116L+D123N+N126D+T131I+S181*+T182*+F202Y+Y242F+E259G+N279S+G303R+E390A+G475K, H1*+G7A+F116L+D123N+N126D+T131 I+R180*+S181*+F202Y+Y242F+E259G+N279S+G303R+E390A+G475K, H1*+G7A+D118T+W140Y+R180*+S181*+T182D+N194F+L205Y+Y242F+E259G+N279S+G303R+E390A+G475K, H1*+G7A+F116N+W140Y+R180*+S181*+T182D+N194F+L205Y+Y242F+E259G+N279S+G303R+E390A+G475K, H1*+G7A+W140Y+R180*+S181*+E189P+N194F+L205Y+Y242F+E259G+N279S+K280Y+G303R+E390A+G475K, H1*+G7A+W140Y+R180*+S181*+T182D+E189P+N194F+L205Y+Y242F+E259G+N279S+G303R+E390A+G475K, H1*+G7A+T134D+W140Y+R180*+S181*+E189P+N194F+L205V+Y242F+E259G+N279S+G303R+E390A+G475K, H1*+G7A+D118T+W140Y+R180*+S181*+T182S+N194F+L205Y+Y242F+E259G+N279S+G303R+E390A+G475K, H1*+G7A+F116N+W140Y+R180*+S181*+T182S+N194F+L205Y+Y242F+E259G+N279S+G303R+E390A+G475K, H1*+G7A+W140Y+R180*+S181*+T182S+E189P+N194F+L205Y+Y242F+E259G+N279S+G303R+E390A+G475K, H1*+G7A+W140Y+R180*+S181*+T182S+E189P+N194F+L205V+Y242F+E259G+N279S+G303R+E390A+G475K, H1*+G7A+D123N+W140Y+I176V+R180*+S181*+N194F+L205V+Y242F+E259G+N279S+G303R+E390A+G475K, H1*+G7A+D123N+W140Y+I176V+R180*+S181*+N194F+L205Y+Y242F+E259G+N279S+G303R+E390A+G475K, H1*+G7A+Q98L+G110K+S132D+Y135E+R180*+S181*+Y242F+E259G+N279S+G303R+T315A+E390A+G475K, H1*+A60V+Q98L+W140Y+R180*+S181*+N194F+L205Y+Y242F+E259G+N279S+G303R+A383D+E390A+G475K, H1*+G7A+W140Y+T151A+R180*+S181*+N194F+L205Y+Y242F+K258V+E259V+N279S+G303R+E390A+G475K, H1*+N3S+G7A+N94D+Q98L+G110K+R180*+S181*+Y242F+V252I+E259G+N279S+G303R+E390A+G475K, H1*+G7A+W140Y+T151A+R180*+S181*+N194F+L205Y+Y242F+K258V+E259V+N279S+G303R+E390A+G475K, H1*+G7A+F116N+W140Y+R180*+S181*+N194F+L205Y+Y242F+E259G+F266Y+N279S+G303R+E390A+G475K, H1*+G7A+F116N+W140Y+R180*+S181*+A185Q+N194F+L205Y+Y242F+E259G+N279S+G303R+E390A+G475K, H1*+G7A+D118T+W140Y+R180*+S181*+E189P+N194F+Y242F+E259G+N279S+K280Y+G303R+E390A+G475K, H1*+G7A+D118T+W140Y+R180*+S181*+N194F+L205Y+Y242F+E259G+F266Y+N279S+G303R+E390A+G475K, H1*+G7A+D118T+W140Y+R180*+S181*+A185Q+N194F+L205Y+Y242F+E259G+N279S+G303R+E390A+G475K, H1*+G7A+W140Y+R180*+S181*+E189P+N194F+L205V+Y242F+E259G+N279S+K280Y+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+T182S+E189P+N194F+Y242F+E259G+N279S+K280Y+G303R+E390A+G475K, H1*+G7A+W140Y+R180*+S181*+T182D+N194F+L205V+Y242F+E259G+F266Y+N279S+G303R+E390A+G475K, H1*+G7A+W140Y+R180*+S181*+A185Q+N194F+L205V+Y242F+E259G+F266Y+N279S+G303R+E390A+G475K, H1*+G7A+W140Y+R180*+S181*+T182D+A185Q+N194F+L205V+Y242F+E259G+N279S+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+T182S+E189P+N194F+Y242F+E259G+N279S+K280Y+G303R+E390A+G475K, H1*+G7A+W140Y+R180*+S181*+T182D+N194F+L205Y+Y242F+E259G+F266Y+N279S+G303R+E390A+G475K, H1*+G7A+W140Y+R180*+S181*+A185Q+N194F+L205Y+Y242F+E259G+F266Y+N279S+G303R+E390A+G475K, H1*+G7A+W140Y+R180*+S181*+T182D+A185Q+N194F+L205Y+Y242F+E259G+N279S+G303R+E390A+G475K, H1*+G7A+F116N+W140Y+R180*+S181*+E189P+N194F+Y242F+E259G+N279S+K280Y+G303R+E390A+G475K, H1*+G7A+W140Y+R180*+S181*+T182D+Y242F+S243Q+E259G+F266Y+N279S+K280Y+G303R+E390A+G475K, H1*+G7A+W140Y+R180*+S181*+T182D+E189P+Y242F+S243Q+E259G+N279S+K280Y+G303R+E390A+G475K, H1*+G7A+W140Y+R180*+S181*+A185Q+Y242F+E259G+F266Y+N279S+K280Y+G303R+E390A+T427L+G475K, H1*+G7A+W140Y+R180*+S181*+A185Q+Y242F+S243Q+E259G+F266Y+N279S+K280Y+G303R+E390A+G475K, H1*+G7A+W140Y+R180*+S181*+T182D+A185Q+Y242F+S243Q+E259G+F266Y+N279S+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+A185Q+N194F+Y242F+S243Q+E259G+N279S+K280Y+G303R+E390A+G475K, H1*+G7A+W140Y+R180*+S181*+T182D+N194F+Y242F+S243Q+E259G+N279S+K280Y+G303R+E390A+G475K, H1*+G7A+W140Y+R180*+S181*+A185Q+E189P+N194F+Y242F+E259G+N279S+K280Y+G303R+E390A+G475K, H1*+G7A+W140Y+R180*+S181*+E189P+L205Y+Y242F+S243Q+E259G+N279S+K280Y+G303R+E390A+G475K, H1*+G7A+W140Y+R180*+S181*+T182D+A185Q+N194F+Y242F+S243Q+E259G+N279S+G303R+E390A+G475K, H1*+G7A+W140Y+R180*+S181*+T182D+N194F+Y242F+S243Q+E259G+F266Y+N279S+G303R+E390A+G475K, H1*+G7A+W140Y+R180*+S181*+E189P+N194F+Y242F+E259G+F266Y+N279S+K280Y+G303R+E390A+G475K, H1*+G7A+W140Y+R180*+S181*+T182D+A185Q+N194F+Y242F+E259G+F266Y+N279S+G303R+E390A+G475K, H1*+G7A+W140Y+R180*+S181*+A185Q+N194F+Y242F+E259G+F266Y+N279S+K280Y+G303R+E390A+G475K, H1*+G7A+W140Y+R180*+S181*+A185Q+E189P+L205Y+Y242F+E259G+N279S+K280Y+G303R+E390A+G475K, H1*+G7A+W140Y+R180*+S181*+E189P+N194F+Y242F+E259G+F266Y+N279S+K280Y+S302T+E390A+G475K, H1*+G7A+W140Y+R180*+S181*+E189P+N194F+Y242F+E259G+M260D+N279S+K280Y+S302T+E390A+G475K, H1*+G7A+W140Y+R180*+S181*+E189P+N194F+Y242F+E259G+F266Y+N279S+K280Y+G303R+E390A+G476A and H1*+G7A+W140Y+R180*+S181*+E189P+N194F+Y242F+E259G+M260W+N279S+K280Y+G303R+E390A+G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding: H1*+G7A+D118T+W140Y+R180*+S181*+E189P+N194F+L205Y+Y242F+E259G+N279S+K280Y+G303R+E390A+G475K, H1*+G7A+F116N+W140Y+R180*+S181*+E189P+N194F+L205Y+Y242F+E259G+N279S+K280Y+G303R+E390A+G475K, H1*+G7A+F116N+W140Y+R180*+S181*+T182D+E189P+N194F+L205Y+Y242F+E259G+N279S+G303R+E390A+G475K, H1*+G7A+G113S+F116W+D123N+W140Y+R180*+S181*+N194F+L205V+Y242F+E259G+N279S+G303R+E390A+G475K, H1*+G7A+N106D+A109G+W140Y+R180*+S181*+N194F+F202Y+L205V+Y242F+E259G+N279S+G303R+E390A+G475K, H1*+G7A+G113S+F116W+D123N+W140Y+R180*+S181*+N194F+L205Y+Y242F+E259G+N279S+G303R+E390A+G475K, H1*+G7A+F116N+W140Y+R180*+S181*+T182D+E189P+N194F+Y242F+E259G+N279S+K280Y+G303R+E390A+G475K, H1*+G7A+F116N+W140Y+R180*+S181*+T182S+E189P+N194F+Y242F+E259G+N279S+K280Y+G303R+E390A+G475K, H1*+G7A+F116N+W140Y+R180*+S181*+T182D+N194F+L205Y+Y242F+E259G+F266Y+N279S+G303R+E390A+G475K, H1*+G7A+F116N+W140Y+R180*+S181*+A185Q+N194F+L205Y+Y242F+E259G+F266Y+N279S+G303R+E390A+G475K, H1*+G7A+F116N+W140Y+R180*+S181*+T182D+A185Q+N194F+L205Y+Y242F+E259G+N279S+G303R+E390A+G475K, H1*+G7A+D118T+W140Y+R180*+S181*+T182D+N194F+L205Y+Y242F+E259G+F266Y+N279S+G303R+E390A+G475K, H1*+G7A+D118T+W140Y+R180*+S181*+A185Q+N194F+L205Y+Y242F+E259G+F266Y+N279S+G303R+E390A+G475K, H1*+G7A+D118T+W140Y+R180*+S181*+T182D+A185Q+N194F+L205Y+Y242F+E259G+N279S+G303R+E390A+G475K, H1*+G7A+W140Y+R180*+S181*+T182D+E189P+N194F+L205V+Y242F+E259G+N279S+K280Y+G303R+E390A+G475K, H1*+G7A+W140Y+R180*+S181*+T182D+A185Q+N194F+L205V+Y242F+E259G+F266Y+N279S+G303R+E390A+G475K, H1*+G7A+W140Y+R180*+S181*+T182D+E189P+N194F+L205Y+Y242F+E259G+N279S+K280Y+G303R+E390A+G475K, H1*+G7A+W140Y+R180*+S181*+T182S+E189P+N194F+L205Y+Y242F+E259G+N279S+K280Y+G303R+E390A+G475K, H1*+G7A+W140Y+R180*+S181*+T182D+A185Q+N194F+L205Y+Y242F+E259G+F266Y+N279S+G303R+E390A+G475K, H1*+G7A+D118T+W140Y+R180*+S181*+T182D+E189P+N194F+Y242F+E259G+N279S+K280Y+G303R+E390A+G475K, H1*+G7A+D118T+W140Y+R180*+S181*+T182S+E189P+N194F+Y242F+E259G+N279S+K280Y+G303R+E390A+G475K, H1*+G7A+W140Y+R180*+S181*+T182D+A185Q+N194F+Y242F+S243Q+E259G+N279S+K280Y+G303R+E390A+G475K, H1*+G7A+W140Y+R180*+S181*+A185Q+N194F+Y242F+S243Q+E259G+F266Y+N279S+K280Y+G303R+E390A+G475K, H1*+G7A+W140Y+R180*+S181*+T182D+E189P+L205Y+Y242F+E259G+F266Y+N279S+K280Y+G303R+E390A+G475K, H1*+G7A+W140Y+R180*+S181*+A185Q+E189P+N194F+Y242F+E259G+F266Y+N279S+K280Y+G303R+E390A+G475K, H1*+G7A+W140Y+R180*+S181*+T182D+E189P+N194F+Y242F+S243Q+E259G+N279S+K280Y+G303R+E390A+G475K, H1*+D118T+W140Y+R180*+S181*+T182S+E189P+N194F+L205Y+Y242F+E259G+N279S+K280Y+G303R+E390A+G475K, H1*+G7A+W140Y+R180*+S181*+T182S+E189P+N194F+L205Y+Y242F+E259G+N279S+K280Y+G303R+E390A+G475K, H1*+G7A+D118T+R180*+S181*+T182S+E189P+N194F+L205Y+Y242F+E259G+N279S+K280Y+G303R+E390A+G475K, H1*+G7A+D118T+W140Y+R180*+S181*+E189P+N194F+L205Y+Y242F+E259G+N279S+K280Y+G303R+E390A+G475K, H1*+G7A+D118T+W140Y+R180*+S181*+T182S+N194F+L205Y+Y242F+E259G+N279S+K280Y+G303R+E390A+G475K, H1*+G7A+D118T+W140Y+R180*+S181*+T182S+E189P+L205Y+Y242F+E259G+N279S+K280Y+G303R+E390A+G475K, H1*+G7A+D118T+W140Y+R180*+S181*+T182S+E189P+N194F+Y242F+E259G+N279S+K280Y+G303R+E390A+G475K, H1*+G7A+D118T+W140Y+R180*+S181*+T182S+E189P+N194F+L205Y+E259G+N279S+K280Y+G303R+E390A+G475K, H1*+G7A+D118T+W140Y+R180*+S181*+T182S+E189P+N194F+L205Y+Y242F+N279S+K280Y+G303R+E390A+G475K, H1*+G7A+D118T+W140Y+R180*+S181*+T182S+E189P+N194F+L205Y+Y242F+E259G+K280Y+G303R+E390A+G475K, H1*+G7A+D118T+W140Y+R180*+S181*+T182S+E189P+N194F+L205Y+Y242F+E259G+N279S+G303R+E390A+G475K, H1*+G7A+D118T+W140Y+R180*+S181*+T182S+E189P+N194F+L205Y+Y242F+E259G+N279S+K280Y+E390A+G475K, H1*+G7A+D118T+W140Y+R180*+S181*+T182S+E189P+N194F+L205Y+Y242F+E259G+N279S+K280Y+G303R+G475K, H1*+G7A+D118T+W140Y+R180*+S181*+T182S+E189P+N194F+L205Y+Y242F+E259G+N279S+K280Y+G303R+E390A, H1*+G7A+D118T+W140Y+R180*+S181*+T182S+E189P+N194F+Y242F+E259G+N279S+K280Y+S302T+E390A+G475K, H1*+G7A+D118T+W140Y+R180*+S181*+T182D+E189P+N194F+Y242F+E259G+N279S+K280Y+G303R+E390A+G476A, H1*+G7A+R37N+W140Y+R180*+S181*+E189P+N194F+Y242F+E259G+F266Y+N279S+K280Y+S302Y+E390A+G475K, H1*+G7A+D118T+W140Y+R180*+S181*+T182S+E189P+N194F+T224Y+Y242F+N279S+K280Y+G303R+E390A+G476A, H1*+G7A+D118T+W140Y+R180*+S181*+T182D+E189P+N194F+T224Y+Y242F+N279S+K280Y+G303R+E390A+G476A, H1*+G7A+D118T+W140Y+R180*+S181*+T182D+A185Q+Y242F+S243Q+E259G+F266Y+N279S+G303R+E390A+G475K, H1*+G7A+D118T+W140Y+R180*+S181*+T182D+N194F+Y242F+S243Q+E259G+F266Y+N279S+G303R+E390A+G475K, H1*+G7A+D118T+W140Y+R180*+S181*+T182D+E189P+N194F+T224Y+Y242F+N279S+K280Y+G303R+E390A+G476A, H1*+G7A+D118T+W140Y+R180*+S181*+T182S+E189P+N194F+T224Y+Y242F+N279S+K280Y+G303R+E390A+G476A, H1*+G7A+D118T+W140Y+R180*+S181*+T182D+A185Q+N194F+Y242F+E259G+N279S+K280Y+S302T+E390A+G475K, H1*+G7A+W140Y+R180*+S181*+E189P+N194F+Y242F+E259G+M260D+N279S+K280Y+S302Y+E390A+N470A+G475K, H1*+G7A+S132L+W140Y+R180*+S181*+E189P+N194F+Y242F+E259G+M260D+N279S+K280Y+G303R+E390A+G475K, and T40N+F116L+D123N+N126D+N128Y+T131I+F155W+R180*+S181*+T182D+E189P+F202Y+K457N+P458S+G475K+G476E, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to H1*+G7A+G113A+F116W+D123N+T131V+Q136K+E169Q+K172Q+R180*+S181*+Y242F+E259G+N279S+G303R+E390A+G475K, H1*+G7A+F116N+W140Y+R180*+S181*+T182S+E189P+N194F+L205Y+Y242F+E259G+N279S+K280Y+G303R+E390A+G475K, H1*+N3S+G7A+N94D+Q98L+W140Y+R180*+S181*+N194F+L205Y+Y242F+V252+E259G+N279S+G303R+E390A+G475K, H1*+G7A+F116N+W140Y+R180*+S181*+T182D+E189P+N194F+L205Y+Y242F+E259G+N279S+K280Y+G303R+E390A+G475K, H1*+G7A+F116N+W140Y+R180*+S181*+T182D+A185Q+N194F+L205Y+Y242F+E259G+F266Y+N279S+G303R+E390A+G475K, H1*+G7A+D118T+W140Y+R180*+S181*+T182D+E189P+N194F+L205Y+Y242F+E259G+N279S+K280Y+G303R+E390A+G475K, H1*+G7A+D118T+W140Y+R180*+S181*+T182D+A185Q+N194F+L205Y+Y242F+E259G+F266Y+N279S+G303R+E390A+G475K, H1*+G7A+D118T+W140Y+R180*+S181*+T182D+E189P+N194F+L205Y+Y242F+E259G+N279S+K280Y+G303R+E390A+G475K, H1*+G7A+W140Y+R180*+S181*+T182D+A185Q+E189P+N194F+Y242F+S243Q+E259G+N279S+K280Y+G303R+E390A+G475K, H1*+G7A+W140Y+R180*+S181*+A185Q+E189P+N194F+Y242F+S243Q+E259G+F266Y+N279S+K280Y+G303R+E390A+G475K, H1*+G7A+W140Y+R180*+S181*+T182D+A185Q+E189P+N194F+Y242F+E259G+F266Y+N279S+K280Y+G303R+E390A+G475K, H1*+G7A+W140Y+R180*+S181*+A185Q+E189P+N194F+L205Y+Y242F+S243Q+E259G+N279S+K280Y+G303R+E390A+G475K, H1*+G7A+W140Y+R180*+S181*+T182D+E189P+L205Y+Y242F+S243Q+E259G+N279S+K280Y+G303R+E390A+I409L+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+S302Y+N394W+G475K, H1*+G7A+D118T+W140Y+R180*+S181*+T182S+E189P+N194F+L205Y+Y242F+E259G+N279S+K280Y+S302T+N394W+G475K, H1*+G7A+D118T+W140Y+R180*+S181*+T182S+E189P+N194F+L205Y+Y242F+E259G+N279S+K280Y+S302T+E390A+G476A, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302Y+E390A+G475K H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+G303R+E390A+N474L, H1*+G7A+D118T+W140Y+R180*+S181*+T182S+E189P+N194F+L205Y+T224Y+Y242F+N279S+K280Y+G303R+E390A+G476A H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302Y+E390A+N474F, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+G475K, H1*+G7A+D118T+W140Y+R180*+S181*+T182S+E189P+N194F+L205V+Y242F+E259G+N279S+K280Y+G303R+E390A+G475K, H1*+G7A+D118T+W140Y+R180*+S181*+T182S+E189P+N194F+L205N+Y242F+E259G+N279S+K280Y+G303R+E390A+G475K, H1*+G7A+D118T+W140Y+R180*+S181*+T182S+E189P+N194F+L205Q+Y242F+E259G+N279S+K280Y+G303R+E390A+G475K, H1*+G7A+D118T+W140Y+R180*+S181*+A185Q+E189P+N194F+L205Y+Y242F+E259G+N279S+K280Y+G303R+E390A+G475K, H1*+G7A+D118T+W140Y+R180*+S181*+T182S+A185Q+E189P+Y242F+E259G+F266Y+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+N474F, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+N394D+G475K, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302T+N394W+G475K, H1*+G7A+D118T+W140Y+R180*+S181*+T182S+E189P+N194F+L205Y+Y242F+E259G+N279S+K280Y+S302T+N394D+G475K, H1*+G7A+D118T+W140Y+R180*+S181*+T182S+A185D+E189P+Y242F+E259G+F266Y+N279S+K280Y+G303R+E390A+G475K, H1*+G7A+D118T+W140Y+R180Q+S181*+T182*+A185Q+E189P+Y242F+E259G+F266Y+N279S+K280Y+G303R+E390A+G475K, H1*+G7A+D118T+W140Y+R180*+S181*+T182S+E189P+N194F+Y242F+E259G+M260D+N279S+K280Y+S302T+E390A+G475K, H1*+G7A+R37N+D118T+W140Y+R180*+S181*+T182S+E189P+N194F+Y242F+E259G+N279S+K280Y+S302Y+E390A+G475K, H1*+G7A+D118T+W140Y+R180*+S181*+T182S+E189P+N194F+Y242F+E259G+M260W+N279S+K280Y+G303R+E390A+G476A, H1*+G7A+D118T+W140Y+R180*+S181*+T182D+E189P+N194F+Y242F+E259G+M260D+N279S+K280Y+S302T+E390A+G475K, H1*+G7A+R37N+D118T+W140Y+R180*+S181*+T182D+E189P+N194F+Y242F+E259G+N279S+K280Y+S302Y+E390A+G475K, H1*+G7A+D118T+W140Y+R180*+S181*+T182S+A185Q+E189P+N194F+Y242F+E259G+N279S+K280Y+G303R+E390A+G476A, H1*+G7A+D118T+W140Y+R180*+S181*+T182S+E189P+N194F+T224Y+Y242F+F266Y+N279S+K280Y+G303R+E390A+G476A, H1*+G7A+D118T+W140Y+R180*+S181*+T182S+E189P+N194F+Y242F+E259G+F266Y+N279S+K280Y+S302T+E390A+G475K, H1*+G7A+D118T+W140Y+R180*+S181*+T182S+E189P+N194F+Y242F+E259G+F266Y+N279S+K280Y+G303R+E390A+G476A, H1*+G7A+D118T+W140Y+R180*+S181*+T182D+E189P+N194F+Y242F+E259G+F266Y+N279S+K280Y+S302T+E390A+G475K, H1*+N3D+G7A+W140Y+R180*+S181*+E189P+N194F+Y242F+E259G+F266Y+N279S+K280Y+S302T+E390A+H420A+G475K, H1*+G7A+D118T+W140Y+R180*+S181*+T182D+E189P+N194F+Y242F+E259G+M260W+N279S+K280Y+G303R+E390A+G476A, H1*+G7A+D118T+W140Y+R180*+S181*+T182S+A185Q+E189P+N194F+T224Y+Y242F+N279S+K280Y+G303R+E390A+G476A, H1*+G7A+D118T+W140Y+R180*+S181*+T182S+A185Q+E189P+N194F+Y242F+E259G+N279S+K280Y+S302T+E390A+G475K, H1*+G7A+D118T+W140Y+R180*+S181*+T182D+E189P+N194F+Y242F+E259G+F266Y+N279S+K280Y+G303R+E390A+G476A, H1*+G7A+D118T+W140Y+R180*+S181*+T182D+A185Q+E189P+N194F+T224Y+Y242F+N279S+K280Y+G303R+E390A+G476A and H1*+G7A+D118T+W140Y+R180*+S181*+T182S+A185Q+E189P+N194F+Y242F+E259G+N279S+K280Y+S302T+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to H1*+G7A+F116L+D123N+N126D+T131I+W140Y+R180*+S181*+N194F+F202Y+L205V+Y242F+E259G+N279S+G303R+E390A+G475K, H1*+G7A+F116L+D123N+N126D+T131I+W140Y+R180*+S181*+N194F+F202Y+L205Y+Y242F+E259G+N279S+G303R+E390A+G475K, H1*+G7A+W140Y+R180*+S181*+T182D+A185Q+E189P+N194F+L205Y+Y242F+S243Q+E259G+N279S+K280Y+G303R+E390A+G475K, H1*+G7A+W140Y+R180*+S181*+A185Q+E189P+N194F+L205Y+Y242F+S243Q+E259G+F266Y+N279S+K280Y+G303R+E390A+G475K, H1*+G7A+W140Y+R180*+S181*+T182D+A185Q+E189P+N194F+L205Y+Y242F+E259G+F266Y+N279S+K280Y+G303R+E390A+G475K, H1*+N3D+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+H420A+G475K, H1*+G7A+D118T+W140Y+R180*+S181*+T182S+E189P+N194F+L205Y+Y242F+E259G+N279S+K280Y+S302Y+E390A+H420A+G475K, H1*+N3D+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+H420A+G475K, H1*+G7A+D118T+W140Y+R180*+S181*+T182S+E189P+N194F+L205Y+Y242F+E259G+N279S+K280Y+G303R+E390A+H420A+G476A, H1*+N3D+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+H420A+N474L, H1*+G7A+D118T+W140Y+R180*+S181*+T182S+E189P+N194F+L205Y+Y242F+E259G+N279S+K280Y+G303R+N394W+H420P+N474L, H1*+G7A+G73L+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302Y+E390A+G475K, H1*+G7A+R37N+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302T+E390A+G476H, H1*+G7A+D118T+W140Y+R180*+S181*+T182S+E189P+N194F+L205Y+Y242F+E259G+N279S+K280Y+G303R+N394D+H420A+G475K, H1*+G7A+D118T+W140Y+R180*+S181*+T182S+E189P+N194F+L205Y+Y242F+E259G+N279S+K280Y+G303R+E390A+H420P+G475K, N3G+G7A+D118T+W140Y+R180*+S181*+T182S+E189P+N194F+L205Y+Y242F+E259G+N279S+K280Y+G303R+E390A+H420A+N474L, H1*+G7A+D118T+Y135E+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+G303R+N394W+H420A+G475K, H1*+G7A+V103I+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+M260D+N279S+K280Y+S302Y+E390A+G475K, H1*+G7A+D118T+W140Y+P146H+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+F261P+N279S+K280Y+G303R+E390A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+Q98L+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+G303R+P321H+E390A+G475K, H1*+G7A+D118T+W140Y+R180*+S181*+T182S+E189P+N194F+L205Y+Y242F+E259G+N279S+K280Y+G303R+P321H+E390A+N474L, H1*+G7A+D118T+S132D+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+G303R+N394D+R414M+G475K, H1*+G7A+T40G+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+M260D+N279S+K280Y+S302T+E390A+G475K, H1*+G7A+D118T+W140Y+R180*+S181*+T182S+E189P+N194F+L205Y+Y242F+E259G+M260W+N279S+K280Y+S302Y+E390A+G476A, H1*+G7A+D118T+W140Y+R180*+S181*+T182S+E189P+N194F+L205Y+Y242F+E259G+M260W+N279S+K280Y+G303R+E390A+G476A, H1*+G7A+D118T+W140Y+R180*+S181*+T182S+E189P+N194F+L205Y+Y242F+E259G+M260W+N279S+K280Y+G303R+E390A+G476A, H1*+G7A+D118T+W140Y+R180*+S181*+T182S+E189P+N194F+L205Y+Y242F+E259G+M260W+N279S+K280Y+S302Y+E390A+G475K, H1*+G7A+D118T+W140Y+R180*+S181*+T182S+E189P+N194F+L205Y+T224Y+Y242F+E259G+N279S+K280Y+G303R+E390A+N474L, H1*+G7A+D118T+W140Y+R180*+S181*+T182S+E189P+N194F+L205Y+Y242F+E259G+N279S+K280Y+S302Y+E390A+H420A+G475K, H1*+G7A+D118T+W140Y+P146H+R180*+S181*+T182S+E189P+N194F+L205Y+Y242F+E259G+N279S+K280Y+S302T+E390A+G475K, H1*+N3G+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+A185Q+E189P+N194F+L205Y+Y242F+E259G+N279S+K280Y+G303R+E390A+G475K, H1*+G7A+D118T+W140Y+R180*+S181*+T182S+E189P+N194F+L205Y+Y242F+E259G+F266Y+N279S+K280Y+G303R+E390A+G475K, H1*+G7A+D118T+W140Y+R180*+S181*+T182S+A185D+E189P+N194F+Y242F+E259G+F266Y+N279S+K280Y+G303R+E390A+G475K, H1*+G7A+D118T+W140Y+R180*+S181*+T182S+A185D+E189P+Y242F+S243Q+E259G+F266Y+N279S+K280Y+G303R+E390A+G475K, H1*+G7A+D118T+W140Y+R180Q+S181*+T182*+A185Q+E189P+Y242F+S243Q+E259G+F266Y+N279S+K280Y+G303R+E390A+G475K, H1*+G7A+D118T+W140Y+R180*+S181*+T182S+A185Q+E189P+L205Y+Y242F+E259G+F266Y+N279S+K280Y+G303R+E390A+G475K, H1*+G7A+D118T+W140Y+R180*+S181*+T182S+A185D+E189P+L205Y+Y242F+E259G+F266Y+N279S+K280Y+G303R+E390A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+T40G+D118T+W140Y+R180*+S181*+T182S+E189P+N194F+L205Y+Y242F+E259G+N279S+K280Y+G303R+E390A+G476A, H1*+G7A+T40G+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+G303R+N394D+N422V+G475K, H1*+G7A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+F261P+N279S+K280Y+G303R+E390A+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+T256D+N279S+K280Y+G303R+E390A+N470M+G475K, H1*+N3D+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+S302T+Y362V+E390A+G475K, H1*+G7A+D118T+W140Y+R180*+S181*+T182S+E189P+N194F+L205Y+Y242F+E259G+N279S+K280Y+G303R+Y362V+E390A+G476A, H1*+G7A+D118T+W140Y+R180*+S181*+T182S+E189P+N194F+L205Y+Y242F+E259G+M260D+N279S+K280Y+G303R+E390A+N474L, H1*+G7A+D118T+W140Y+R180*+S181*+T182S+E189P+N194F+L205Y+T224Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+Y362V+E390A+G475K, H1*+G7A+D118T+W140Y+R180*+S181*+T182S+E189P+N194F+L205Y+Y242F+E259G+M260W+N279S+K280Y+S302T+E390A+G475K, H1*+G7A+D118T+W140Y+R180*+S181*+T182S+E189P+N194F+L205Y+T224Y+Y242F+E259G+N279S+K280Y+G303R+E390A+G476A, H1*+G7A+D118T+W140Y+R180*+S181*+T182S+E189P+N194F+L205Y+Y242F+E259G+M260D+N279S+K280Y+G303R+E390A+N474F, H1*+G7A+D118T+W140Y+R180*+S181*+T182S+E189P+N194F+L205Y+Y242F+E259G+N279S+K280Y+G303R+E390A+H420P+G475K, H1*+G7A+D118T+W140Y+R180*+S181*+T182S+E189P+N194F+L205Y+Y242F+E259G+N279S+K280Y+G303R+E390A+N470M+G476A, H1*+G7A+D118T+W140Y+R180*+S181*+T182S+E189P+N194F+L205Y+Y242F+E259G+N279S+K280Y+S302T+E390A+H420A+G475K, H1*+G7A+D118T+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+N279S+K280Y+G303R+N394W+H420P+G475K, H1*+G7A+D118T+W140Y+R180*+S181*+T182S+E189P+N194F+L205Y+Y242F+E259G+N279S+K280Y+S302T+H323W+E390A+G475K, H1*+G7A+R37A+D118T+W140Y+R180*+S181*+T182S+E189P+N194F+L205Y+Y242F+E259G+N279S+K280Y+S302Y+E390A+N474L, H1*+G7A+R37A+D118T+W140Y+R180*+S181*+T182S+E189P+N194F+L205Y+Y242F+E259G+N279S+K280Y+S302Y+E390A+G475K, H1*+G7A+R37A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302T+N394W+H420A+G475K, H1*+G7A+R37N+D118T+W140Y+R180*+S181*+T182S+E189P+N194F+L205Y+Y242F+E259G+N279S+K280Y+S302Y+N394D+G475K, H1*+G7A+R37N+D118T+W140Y+R180*+S181*+T182S+E189P+N194F+L205Y+Y242F+E259G+N279S+K280Y+S302T+N394D+G475K, H1*+G7A+R37A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302Y+E390A+N474L, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302Y+E390A+G475K, H1*+G7A+R37A+D118T+W140Y+R180*+S181*+T182S+E189P+N194F+L205Y+T224F+Y242F+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+H420A+G475K, H1*+G7A+R37N+D118T+W140Y+R180*+S181*+T182S+E189P+N194F+L205Y+T224Y+Y242F+N279S+K280Y+G303R+E390A+G475K, H1*+G7A+D118T+W140Y+R180*+S181*+T182S+E189P+N194F+L205Y+Y242F+E259G+N279S+K280Y+G303R+N394W+H420A+G475K, H1*+G7A+D118T+W140Y+R180*+S181*+T182S+E189P+N194F+L205Y+Y242F+E259G+N279S+K280Y+G303R+N394W+H420A+N474L, H1*+G7A+D118T+W140Y+P146H+R180*+S181*+T182S+E189P+N194F+L205Y+Y242F+E259G+N279S+K280Y+S302T+E390A+G476A, H1*+G7A+D118T+W140Y+P146H+R180*+S181*+T182S+E189P+N194F+L205Y+Y242F+E259G+N279S+K280Y+S302Y+E390A+N474L, H1*+G7A+D118T+W140Y+R180*+S181*+T182S+A185Q+E189P+N194F+Y242F+E259G+F266Y+N279S+K280Y+G303R+E390A+G475K, H1*+G7A+D118T+W140Y+R180Q+S181*+T182*+A185Q+E189P+N194F+Y242F+E259G+F266Y+N279S+K280Y+G303R+E390A+G475K, H1*+G7A+D118T+W140Y+R180*+S181*+T182S+A185Q+E189P+Y242F+S243Q+E259G+F266Y+N279S+K280Y+G303R+E390A+G475K, H1*+N3D+G7A+D118T+W140Y+R180*+S181*+T182S+E189P+N194F+L205Y+Y242F+E259G+N279S+K280Y+G303R+E390A+G475K, H1*+G7A+D118T+W140Y+R180*+S181*+T182S+E189P+N194F+L205Y+Y242F+E259G+N279S+K280Y+G303R+E390A+N470M+G475K, H1*+G7A+D118T+W140Y+R180*+S181*+T182S+E189P+N194F+L205Y+Y242F+E259G+N279S+K280Y+S302Y+Y371N+E390A+G475K, H1*+G7A+D118T+Y135E+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+T224F+Y242F+E259G+N279S+K280Y+S302T+E390A+G475K, H1*+G7A+D118T+W140Y+R180*+S181*+T182S+E189P+N194F+L205Y+Y242F+E259G+M260D+N279S+K280Y+G303R+N394W+G475K, H1*+G7A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+Y242F+T256D+N279S+K280Y+G303R+E390A+G475K, H1*+G7A+R37A+D118T+W140Y+R180*+S181*+T182S+E189P+N194F+L205Y+T224Y+Y242F+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+E259G+N279S+K280Y+S302T+E390A+N470M+G475K, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+S132L+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+H420A+G476A, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302Y+E390A+G475K, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302T+E390A+G475K, H1*+G7A+D118T+S132D+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+S302Y+N394D+G475K H1*+G7A+D118T+S132L+W140Y+R180*+S181*+T182S+E189P+N194F+L205Y+Y242F+E259G+N279S+K280Y+S302Y+N394W+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+H420A+G476H, H1*+G7A+D118T+W140Y+R180*+S181*+T182S+E189P+N194F+L205Y+T224F+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+S302Y+E390A+G475K, H1*+G7A+D118T+Q138V+W140Y+R180*+S181*+T182S+E189P+N194F+L205Y+Y242F+E259G+N279S+K280Y+S302Y+E390A+G475K, H1*+G7A+D118T+S132L+W140Y+R180*+S181*+T182S+E189P+N194F+L205Y+Y242F+E259G+N279S+K280Y+S302Y+N394W+G475K, H1*+G7A+Q98L+D118T+W140Y+R180*+S181*+T182S+E189P+N194F+L205Y+Y242F+E259G+N279S+K280Y+G303R+E390A+G475K, H1*+G7A+R37N+D118T+W140Y+R180*+S181*+T182S+E189P+N194F+L205Y+Y242F+E259G+N279S+K280Y+G303R+E390A+G475K, H1*+N3D+G7A+D118T+W140Y+R180*+S181*+T182D+E189P+N194F+Y242F+E259G+N279S+K280Y+S302T+E390A+H420A+G475K, H1*+G7A+D118T+W140Y+R180*+S181*+T182S+A185Q+E189P+N194F+L205Y+T224Y+Y242F+N279S+K280Y+G303R+E390A+G476A, H1*+G7A+D118T+W140Y+R180*+S181*+T182S+A185Q+E189P+N194F+Y242F+E259G+M260D+N279S+K280Y+S302T+E390A+G475K, H1*+G7A+R37N+D118T+W140Y+R180*+S181*+T182S+A185Q+E189P+N194F+Y242F+E259G+N279S+K280Y+S302Y+E390A+G475K, H1*+G7A+D118T+W140Y+R180*+S181*+T182S+E189P+N194F+Y242F+E259G+M260D+F266Y+N279S+K280Y+S302T+E390A+G475K, H1*+G7A+D118T+W140Y+R180*+S181*+T182S+E189P+N194F+Y242F+E259G+M260W+F266Y+N279S+K280Y+G303R+E390A+G476A, H1*+G7A+D118T+W140Y+R180*+S181*+T182D+E189P+N194F+Y242F+E259G+M260D+F266Y+N279S+K280Y+S302T+E390A+G475K, H1*+G7A+R37N+D118T+W140Y+R180*+S181*+T182D+E189P+N194F+Y242F+E259G+F266Y+N279S+K280Y+S302Y+E390A+G475K, H1*+G7A+D118T+W140Y+R180*+S181*+T182S+A185Q+E189P+N194F+T224Y+Y242F+F266Y+N279S+K280Y+G303R+E390A+G476A, H1*+G7A+D118T+W140Y+R180*+S181*+T182S+A185Q+E189P+N194F+Y242F+E259G+F266Y+N279S+K280Y+S302T+E390A+G475K, H1*+G7A+D118T+W140Y+R180*+S181*+T182S+A185Q+E189P+N194F+Y242F+E259G+F266Y+N279S+K280Y+G303R+E390A+G476A, H1*+G7A+D118T+W140Y+R180*+S181*+T182S+E189P+N194F+Y242F+E259G+M260D+N279S+K280Y+S302Y+E390A+N470M+G475K, H1*+G7A+D118T+W140Y+R180*+S181*+T182D+E189P+N194F+Y242F+E259G+M260D+N279S+K280Y+S302Y+E390A+N470M+G475K, H1*+G7A+D118T+W140Y+R180*+S181*+T182S+A185Q+E189P+N194F+Y242F+E259G+M260W+N279S+K280Y+G303R+E390A+G476A, H1*+G7A+D118T+W140Y+R180*+S181*+T182D+E189P+N194F+Y242F+E259G+M260D+N279S+K280Y+S302Y+E390A+N470M+G475K, H1*+G7A+D118T+W140Y+R180*+S181*+T182D+A185Q+E189P+N194F+Y242F+E259G+F266Y+N279S+K280Y+G303R+E390A+G475K, H1*+G7A+D118T+W140Y+R180*+S181*+T182S+A185Q+E189P+N196I+Y242F+E259G+F266Y+N279S+K280Y+G303R+E390A+G475K, H1*+G7A+D118T+W140Y+R180*+S181*+T182S+A185D+E189P+Y242F+E259G+F266Y+N279S+K280Y+G303R+E390A+H420Y+G475K, H1*+G7A+D118T+W140Y+R180*+S181*+A185Q+E189P+N194F+L205Y+Y242F+E259G+F266Y+N279S+K280Y+G303R+E390A+G475K, H1*+G7A+D118T+W140Y+R180*+S181*+T182S+E189P+N194F+Y242F+E259G+M260D+N279S+K280Y+S302Y+E390A+N470M+G475K, H1*+G7A+D118T+W140Y+R180*+S181*+T182S+A185Q+E189P+N194F+Y242F+E259G+M260W+N279S+K280Y+G303R+E390A+G476A and H1*+G7A+D118T+W140Y+R180*+S181*+T182D+E189P+N194F+L205Y+T224Y+Y242F+F266Y+N279S+K280Y+G303R+E390A+G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to H1*+N3D+G7A+D118T+W140Y+R180*+S181*+T182S+E189P+N194F+L205Y+Y242F+E259G+N279S+K280Y+S302T+V365I+E390A+N474L, H1*+N3D+G7A+D118T+W140Y+R180*+S181*+T182S+E189P+N194F+L205Y+Y242F+E259G+N279S+K280Y+S302T+E390A+H420A+G475K, H1*+N3D+G7A+D118T+W140Y+R180*+S181*+T182S+E189P+N194F+L205Y+Y242F+E259G+N279S+K280Y+S302T+E390A+H420A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+R37A+D118T+W140Y+R180*+S181*+T182S+E189P+N194F+L205Y+Y242F+E259G+N279S+K280Y+G303R+N394D+N474F, H1*+N3D+G7A+D118T+W140Y+R180*+S181*+T182S+E189P+N194F+L205Y+Y242F+E259G+N279S+K280Y+S302Y+E390A+H420A+G475K, H1*+N3D+G7A+D118T+W140Y+R180*+S181*+T182S+E189P+N194F+L205Y+Y242F+E259G+N279S+K280Y+G303R+E390D+H420P+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+D118T+W140Y+R180*+S181*+T182S+E189P+N194F+L205Y+Y242F+E259G+N279S+K280Y+G303R+N394W+H420A+G475K, H1*+G7A+R37N+D118T+W140Y+R180*+S181*+T182S+E189P+N194F+L205Y+Y242F+E259G+N279S+K280Y+G303R+E390A+H420A+G475K, H1*+N3D+G7A+D118T+W140Y+R180*+S181*+T182S+E189P+N194F+L205Y+Y242F+E259G+N279S+K280Y+G303R+E390A+H420A+G475K, H1*+N3D+G7A+D118T+W140Y+R180*+S181*+T182S+E189P+N194F+L205Y+Y242F+E259G+N279S+K280Y+G303R+E390A+H420A+N474L, H1*+G7A+R37N+D118T+W140Y+R180*+S181*+T182S+E189P+N194F+L205Y+Y242F+E259G+N279S+K280Y+G303R+N394W+H420A+G475K, H1*+N3D+G7A+D118T+W140Y+R180*+S181*+T182S+E189P+N194F+L205Y+Y242F+E259G+N279S+K280Y+G303R+E390A+H420P+G475K, H1*+G7A+D118T+Q138V+W140Y+R180*+S181*+T182S+E189P+N194F+L205Y+Y242F+E259G+N279S+K280Y+G303R+H323W+E390A+N474L, H1*+G7A+D118T+W140Y+R180*+S181*+T182S+E189P+N194F+L205Y+T224F+Y242F+E259G+N279S+K280Y+G303R+N394W+H420A+G475K, H1*+G7A+D118T+W140Y+R180*+S181*+T182S+E189P+N194F+L205Y+Y242F+E259G+N279S+K280Y+G303R+P321H+Q360Y+E390A+G476A, H1*+G7A+D118T+W140Y+R180*+S181*+T182S+E189P+N194F+L205Y+Y242F+E259G+M260D+N279S+K280Y+S302Y+E390A+N470M+G475K, H1*+N3D+G7A+D118T+W140Y+R180*+S181*+T182S+E189P+N194F+L205Y+Y242F+E259G+N279S+K280Y+S302T+E390A+H420A+G475K, H1*+G7A+D118T+S132L+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+A185Q+E189P+N194F+L205Y+Y242F+E259G+F266Y+N279S+K280Y+G303R+E390A+G475K, H1*+G7A+D118T+W140Y+R180*+S181*+T182S+A185Q+E189P+N194F+L205N+Y242F+E259G+F266Y+N279S+K280Y+G303R+E390A+G475K, H1*+G7A+D118T+W140Y+R180Q+S181*+T182*+A185Q+E189P+N194F+L205Y+Y242F+E259G+F266Y+N279S+K280Y+G303R+E390A+G475K, H1*+N3D+G7A+D118T+W140Y+R180*+S181*+T182S+E189P+N194F+L205Y+Y242F+E259G+N279S+K280Y+S302T+E390A+H420A+G475K, H1*+G7A+Q98L+D118T+W140Y+R180*+S181*+T182S+E189P+N194F+L205Y+Y242F+E259G+N279S+K280Y+G303R+N394D+H420A+G475K, H1*+G7A+D118T+W140Y+R180*+S181*+T182S+E189P+N194F+L205Y+Y242F+E259G+M260D+N279S+K280Y+G303R+Y362V+E390A+G475K, H1*+G7A+D118T+Y135E+W140Y+R180*+S181*+T182S+E189P+N194F+L205Y+T224F+Y242F+E259G+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+Y371N+E390A+G475K, H1*+G7A+D118T+Y135E+W140Y+R180*+S181*+T182S+E189P+N194F+L205Y+Y242F+E259G+N279S+K280Y+G303R+P321Y+E390A+G475K, H1*+G7A+D118T+S132D+W140Y+R180*+S181*+T182S+E189P+N194F+L205Y+Y242F+E259G+N279S+K280Y+G303R+P321Y+E390A+G475K, H1*+G7A+D118T+S132D+W140Y+R180*+S181*+T182S+E189P+N194F+L205Y+Y242F+E259G+N279S+K280Y+G303R+Y362V+E390A+N474L, H1*+G7A+D118T+W140Y+R180*+S181*+T182S+E189P+N194F+L205Y+Y242F+E259G+M260D+N279S+K280Y+G303R+E390A+H420A+G475K, H1*+G7A+D118T+Y135E+W140Y+R180*+S181*+T182S+E189P+N194F+L205Y+Y242F+E259G+N279S+K280Y+G303R+N394W+H420A+G475K, H1*+N3D+G7A+D118T+W140Y+R180*+S181*+T182S+E189P+N194F+L205Y+Y242F+E259G+N279S+K280Y+G303R+N394D+H420A+G475K, H1*+G7A+D118T+Q138V+W140Y+R180*+S181*+T182S+E189P+N194F+L205Y+Y242F+E259G+N279S+K280Y+G303R+E390A+H420Q+G475K, H1*+G7A+R37N+D118T+W140Y+R180*+S181*+T182S+E189P+N194F+L205Y+Y242F+E259G+N279S+K280Y+G303R+E390A+H420P+G476H, H1*+G7A+R37A+Q98L+D118T+W140Y+R180*+S181*+T182S+E189P+N194F+L205Y+Y242F+E259G+N279S+K280Y+S302Y+E390A+G475K, H1*+G7A+R37A+D118T+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+M260D+N279S+K280Y+G303R+E390A+H420P+G476A, H1*+G7A+R37A+D118T+W140Y+R180*+S181*+T182S+E189P+N194F+L205Y+Y242F+E259G+N279S+K280Y+S302Y+E390A+H420A+G475K, H1*+G7A+D118T+Y135E+W140Y+R180*+S181*+T182S+E189P+N194F+L205Y+Y242F+E259G+N279S+K280Y+S302T+Y371N+E390A+G475K, H1*+G7A+D118T+W140Y+R180*+S181*+T182S+E189P+N194F+L205Y+Y242F+E259G+N279S+K280Y+G303R+H323W+E390A+H420A+N474L, H1*+G7A+D118T+Y135E+W140Y+R180*+S181*+T182S+E189P+N194F+L205Y+Y242F+E259G+N279S+K280Y+G303R+E390A+R414M+G476H, H1*+G7A+R37N+D118T+W140Y+R180*+S181*+T182S+E189P+N194F+L205Y+Y242F+E259G+N279S+K280Y+S302Y+E390A+H420A+G475K, H1*+G7A+R37A+D118T+W140Y+R180*+S181*+T182S+E189P+N194F+L205Y+Y242F+E259G+N279S+K280Y+S302T+E390A+H420A+G476A, H1*+G7A+R37N+D118T+W140Y+R180*+S181*+T182S+E189P+N194F+L205Y+Y242F+E259G+N279S+K280Y+S302Y+E390A+H420A+G475K, H1*+G7A+R37A+D118T+W140Y+R180*+S181*+T182S+E189P+N194F+L205Y+Y242F+E259G+N279S+K280Y+S302Y+E390A+H420A+G476H, H1*+G7A+R37A+D118T+W140Y+R180*+S181*+T182S+E189P+N194F+L205Y+Y242F+E259G+N279S+K280Y+S302Y+N394D+H420A+G475K, H1*+G7A+R37N+D118T+W140Y+R180*+S181*+T182S+E189P+N194F+L205Y+Y242F+E259G+N279S+K280Y+S302T+E390A+H420A+G475K, H1*+G7A+R37A+D118T+W140Y+R180*+S181*+T182S+E189P+N194F+L205Y+Y242F+E259G+N279S+K280Y+S302Y+E390A+H420A+G475K, H1*+G7A+R37N+D118T+W140Y+R180*+S181*+T182S+E189P+N194F+L205Y+Y242F+E259G+N279S+K280Y+S302T+E390A+H420A+G475K, H1*+G7A+R37A+D118T+W140Y+R180*+S181*+T182S+E189P+N194F+L205Y+Y242F+E259G+N279S+K280Y+S302T+E390A+H420A+G475K, H1*+G7A+R37N+D118T+W140Y+R180*+S181*+T182S+E189P+N194F+L205Y+Y242F+E259G+N279S+K280Y+S302Y+E390A+H420A+G476H, H1*+G7A+R37N+D118T+W140Y+R180*+S181*+T182S+E189P+N194F+L205Y+Y242F+E259G+N279S+K280Y+S302T+N394D+H420A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+R37A+D118T+W140Y+R180*+S181*+T182S+E189P+N194F+L205Y+Y242F+E259G+N279S+K280Y+G303R+E390A+G475K, H1*+G7A+R37A+D118T+W140Y+R180*+S181*+T182S+E189P+N194F+L205Y+Y242F+E259G+M260D+N279S+K280Y+G303R+N394W+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+Y242F+E259G+M260D+N279S+K280Y+G303R+N394D+G475K, H1*+G7A+R37N+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+A185Q+E189P+N194F+L205Q+Y242F+E259G+F266Y+N279S+K280Y+G303R+E390A+G475K, H1*+N3G+G7A+V104M+D118T+W140Y+R180*+S181*+T182S+E189P+N194F+L205Y+Y242F+E259G+N279S+K280Y+S302Y+N394W+G475K, H1*+N3G+G7A+D118T+W140Y+R180*+S181*+T182S+E189P+N194F+L205Y+Y242F+E259G+N279S+K280Y+G303R+N394W+H420A+G475K, H1*+N3D+G7A+D118T+W140Y+R180*+S181*+T182S+E189P+N194F+L205Y+Y242F+E259G+N279S+K280Y+G303R+E390A+H420A+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+E390A+N470M+G475K, H1*+G7A+R37A+D118T+W140Y+R180*+S181*+T182S+E189P+N194F+L205Y+Y242F+E259G+M260D+N279S+K280Y+G303R+N394W+G475K, H1*+G7A+R37A+D118T+W140Y+R180*+S181*+T182S+E189P+N194F+L205Y+Y242F+E259G+N279S+K280Y+G303R+E390A+H420A+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+R37A+D118T+W140Y+R180*+S181*+T182S+E189P+N194F+L205Y+T224Y+Y242F+E259G+N279S+K280Y+G303R+N394W+G475K, H1*+G7A+R37A+D118T+W140Y+R180*+S181*+T182S+E189P+N194F+L205Y+Y242F+E259G+F261P+N279S+K280Y+G303R+E390A+G475K, H1*+G7A+R37A+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+T224Y+Y242F+E259G+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+H420A+G475K, H1*+N3D+G7A+D118T+W140Y+R180*+S181*+T182S+E189P+N194F+L205Y+Y242F+E259G+M260D+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3G+G7A+R37N+D118T+W140Y+R180*+S181*+T182S+E189P+N194F+L205Y+Y242F+E259G+N279S+K280Y+G303R+E390A+G475K, H1*+G7A+D118T+W140Y+R180*+S181*+T182S+E189P+N194F+L205Y+T224F+Y242F+E259G+N279S+K280Y+G303R+E390A+H420A+G475K, H1*+G7A+D118T+S132L+W140Y+R180*+S181*+T182S+E189P+N194F+L205Y+T224F+Y242F+E259G+N279S+K280Y+S302Y+E390A+G475K, H1*+G7A+D118T+Q138V+W140Y+R180*+S181*+T182S+E189P+N194F+L205Y+T224F+Y242F+E259G+N279S+K280Y+S302Y+E390A+G475K, H1*+G7A+R37N+D118T+W140Y+R180*+S181*+T182S+E189P+N194F+L205Y+Y242F+T256D+E259G+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+N394D+G475K, N3G+G7A+R37V+D118T+W140M+R180*+S181*+T182S+E189P+N194F+L205Y+Y242F+E259G+N279S+K280Y+G303R+E390A+H420A+G475K, H1*+G7A+R37N+D118T+W140Y+R180*+S181*+T182S+E189P+N194F+L205Y+Y242F+E259G+N279S+K280Y+G303R+H323W+N394D+G475K, H1*+N3D+G7A+D118T+W140Y+R180*+S181*+T182S+E189P+N194F+L205Y+Y242F+E259G+N279S+K280Y+G303R+H323W+E390A+G475K, H1*+N3D+G7A+D118T+W140Y+R180*+S181*+T182S+E189P+N194F+L205Y+Y242F+E259G+N279S+K280Y+G303R+H323W+E390A+G475K, H1*+G7A+D118T+S132L+W140Y+R180*+S181*+T182D+E189P+N194F+L205Y+Y242F+E259G+M260D+N279S+K280Y+G303R+E390A+G475K, H1*+G7A+D118T+W140Y+R180*+S181*+T182S+A185Q+E189P+N194F+Y242F+E259G+M260D+N279S+K280Y+S302Y+E390A+N470M+G475K, H1*+N3D+G7A+D118T+W140Y+R180*+S181*+T182S+A185Q+E189P+N194F+Y242F+E259G+N279S+K280Y+S302T+E390A+H420A+G475K, H1*+G7A+D118T+W140Y+R180*+S181*+T182S+A185Q+E189P+N194F+L205Y+Y242F+E259G+M260W+N279S+K280Y+G303R+E390A+G476A, H1*+G7A+D118T+W140Y+R180*+S181*+T182S+E189P+N194F+Y242F+E259G+M260D+F266Y+N279S+K280Y+S302Y+E390A+N470M+G475K, H1*+G7A+D118T+S132L+W140Y+R180*+S181*+T182S+E189P+N194F+Y242F+E259G+M260D+F266Y+N279S+K280Y+G303R+E390A+G475K, H1*+N3D+G7A+D118T+W140Y+R180*+S181*+T182D+E189P+N194F+Y242F+E259G+F266Y+N279S+K280Y+S302T+E390A+H420A+G475K, H1*+G7A+D118T+W140Y+R180*+S181*+T182S+A185Q+E189P+N194F+Y242F+E259G+M260W+F266Y+N279S+K280Y+G303R+E390A+G476A, H1*+G7A+R37N+D118T+W140Y+R180*+S181*+T182S+A185Q+E189P+N194F+L205Y+Y242F+E259G+N279S+K280Y+S302Y+E390A+G475K, H1*+G7A+D118T+W140Y+R180*+S181*+T182S+A185Q+E189P+N194F+L205Y+Y242F+E259G+M260D+N279S+K280Y+S302T+E390A+G475K and H1*+N3D+G7A+D118T+W140Y+R180*+S181*+T182D+E189P+N194F+L205Y+Y242F+E259G+N279S+K280Y+S302T+E390A+H420A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to H1*+G7A+N106D+A109G+F116W+D123N+N126D+T131I+T165V+N174S+R180*+S181*+T182I+F202Y+Y242F+E259G+N279S+G303R+E390A+G475K, H1*+N3D+G7A+D118T+W140Y+R180*+S181*+T182S+E189P+N194F+L205Y+Y242F+E259G+N279S+K280Y+G303R+Y362V+E390A+H420A+N474L, H1*+N3D+G7A+K93R+D118T+W140Y+R180*+S181*+T182S+E189P+N194F+L205Y+Y242F+E259G+N279S+K280Y+G303R+E390A+H420A+N474L, H1*+N3D+G7A+R37N+D118T+Q138V+W140Y+R180*+S181*+T182S+E189P+N194F+L205Y+Y242F+E259G+N279S+K280Y+G303R+E390A+G475K, H1*+G7A+D118T+Y135E+W140Y+R180*+S181*+T182S+E189P+N194F+L205Y+Y242F+E259G+M260D+N279S+K280Y+G303R+W346N+E390A+G475K, H1*+G7A+D118T+Y135E+W140Y+R180*+S181*+T182S+E189P+N194F+L205Y+Y242F+E259G+F261P+N279S+K280Y+G303R+Y362V+E390A+G475K, H1*+N3G+G7A+D118T+Q138V+W140Y+R180*+S181*+T182S+E189P+N194F+L205Y+Y242F+E259G+N279S+K280Y+G303R+E390A+H420A+G476A, H1*+G7A+R37N+D118T+S132D+W140Y+R180*+S181*+T182S+E189P+N194F+L205Y+Y242F+E259G+N279S+K280Y+G303R+E390A+N470M+G475K, H1*+N3D+G7A+R37A+D118T+W140Y+R180*+S181*+T182S+E189P+N194F+L205Y+Y242F+E259G+N279S+K280Y+S302Y+N394D+H420A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3G+G7A+R37N+D118T+W140Y+R180*+S181*+T182S+E189P+N194F+L205Y+Y242F+E259G+N279S+K280Y+G303R+N394W+H420A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3D+G7A+R37N+D118T+W140Y+R180*+S181*+T182S+E189P+N194F+L205Y+Y242F+E259G+N279S+K280Y+G303R+G361M+H420A+G475K, H1*+N3D+G7A+R37A+D118T+W140Y+R180*+S181*+T182S+E189P+N194F+L205Y+Y242F+E259G+N279S+K280Y+G303R+E390A+H420A+G475K, H1*+N3D+G7A+D118T+S132D+W140Y+R180*+S181*+T182S+E189P+N194F+L205Y+Y242F+E259G+N279S+K280Y+G303R+E390A+H420A+G475K, H1*+G7A+R37A+D118T+W140Y+R180*+S181*+T182S+E189P+N194F+L205Y+Y242F+E259G+M260W+N279S+K280Y+G303R+E390A+N470M+G475K, H1*+G7A+R37N+D118T+W140Y+R180*+S181*+T182S+E189P+N194F+L205Y+Y242F+E259G+M260W+N279S+K280Y+G303R+E390A+H420A+G475K, H1*+G7A+R37N+D118T+W140Y+R180*+S181*+T182S+E189P+N194F+L205Y+Y242F+E259G+M260D+N279S+K280Y+G303R+N394W+H420A+G475K, H1*+G7A+R37A+D118T+W140Y+R180*+S181*+T182S+E189P+N194F+L205Y+Y242F+E259G+M260D+N279S+K280Y+G303R+E390A+N470M+G475K, H1*+G7A+R37A+D118T+W140Y+R180*+S181*+T182S+E189P+N194F+L205Y+Y242F+E259G+M260D+N279S+K280Y+G303R+N394D+H420A+G475K, H1*+G7A+R37N+D118T+W140Y+N174K+R180*+S181*+T182S+E189P+N194F+L205Y+Y242F+E259G+M260D+N279S+K280Y+G303R+N394W+G475K, H1*+G7A+R37A+D118T+W140Y+R180*+S181*+T182S+E189P+N194F+L205Y+Y242F+E259G+N279S+K280Y+G303R+P321Y+L354V+N394D+G475K, H1*+G7A+R37N+D118T+W140Y+R180*+S181*+T182S+E189P+N194F+L205Y+Y242F+E259G+N279S+K280Y+G303R+H323W+Q360M+E390A+G475K, H1*+N3D+G7A+D118T+W140Y+R180*+S181*+T182S+A185Q+E189P+N194F+Y242F+E259G+F266Y+N279S+K280Y+S302T+E390A+H420A+G475K and H1*+G7A+A31S+R37N+D118T+W140Y+R180*+S181*+T182S+A185Q+E189P+N194F+Y242F+E259G+F266Y+N279S+K280Y+S302Y+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to H1*+G7A+D118T+S132L+W140Y+R180*+S181*+T182S+A185Q+E189P+N194F+L205Y+Y242F+E259G+M260D+F266Y+N279S+K280Y+G303R+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to H1*+N54S+V56T+K72R+G113Q+F116Q+D118Q+D123N+N126D+T131 I+K142G+D144N+W167F+K172G+N174S+S181T+T182*+G183*+F202Y+E390A+F395Y+K457R+S472R+V473C+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 1 comprising modifications in the positions corresponding to H1*+N54S+V56T+K72R+N106D+A109G+G113Q+F116Q+D118Q+D123N+N126D+T131I+K142G+D144N+W167F+K172G+N174S+S181T+T182*+G183*+F202Y+E390A+F395Y+K457R+S472R+V473C+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 2 comprising modifications in the positions corresponding to E169I+R180*+S181*, T114K+R180*+S181*, W167A+R180*+S181*, E169V+R180*+S181*, R180*+S181*+W188C, W167D+R180*+S181*, Y177D+R180*+S181*, W167S+R180*+S181*, W167E+R180*+S181*, W167P+R180*+S181*, R180*+S181*+L205R, R180*+S181*+A203F, W159L+R180*+S181*, Y160P+R180*+S181*, W159H+R180*+S181*, K178A+R180*+S181*, R180*+S181*+G183P, Q136T+R180*+S181*, K142P+R180*+S181*, K142E+R180*+S181*, Y152L+R180*+S181*, A111L+R180*+S181*, G110K+R180*+S181*, A111G+R180*+S181* and V117G+R180*+S181*, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 2 comprising modifications in the positions corresponding to R180*+S181*+E193+H323N, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: SEQ ID No: 1, 2, 3, 4, 5, 6, 7, 8, 9, 10, 11, 12, 13 or 14.

In one aspect, the alpha-amylase variant of SEQ ID NO: 2 comprising modifications in the positions corresponding to G7H+Q98L+G110K+R180*+S181*, G7H+W167P+R180*+S181*+G455R and Q98L+V103A+G110K+R180*+S181*, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: SEQ ID No: 1, 2, 3, 4, 5, 6, 7, 8, 9, 10, 11, 12, 13 or 14.

In one aspect, the alpha-amylase variant of SEQ ID NO: 2 comprising modifications in the positions corresponding to A60V+Q98L+G110K+R180*+S181*+A383D, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding to F116L+D123N+N126D+T131I+R180*+S181*+F202Y and Q98L+G110K+S132D+Y135E+R180*+S181*+T315A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14.

In one aspect, the alpha-amylase variant of SEQ ID NO: 2 comprising modifications in the positions corresponding to H1*+G7A+R180*+S181*+Y242F+E259G+N279S+G303R+E390A+G475K, H1*+G7A+S181*+T182*+Y242F+E259G+N279S+G303R+E390A+G475K and H1*+G7S+R180*+S181*+Y242F+E259G+N279S+G303R+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding to H1*+G7A+R180*+S181*+T182S+Y242F+E259G+N279S+G303R+E390A+G475K, H1*+G7A+R180*+T182L+G183*+Y242F+E259G+N279S+G303R+E390A+G475K, H1*+G7A+S181 L+T182*+G183*+Y242F+E259G+N279S+G303R+E390A+G475K, H1*+G7A+R180S+T182*+G183*+Y242F+E259G+N279S+G303R+E390A+G475K, H1*+G7A+A60P+R180*+S181*+Y242F+E259G+N279S+G303R+E390A+G475K, H1*+G7A+M105L+R180*+S181*+Y242F+E259G+N279S+G303R+E390A+G475K, H1*+G7A+M105I+R180*+S181*+Y242F+E259G+N279S+G303R+E390A+G475K, H1*+G7A+R180*+S181*+T182D+Y242F+E259G+N279S+G303R+E390A+G475K, H1*+G7A+R180*+S181*+T182E+Y242F+E259G+N279S+G303R+E390A+G475K, H1*+G7A+R180*+S181*+T182G+Y242F+E259G+N279S+G303R+E390A+G475K, H1*+G7A+R180*+S181*+T182M+Y242F+E259G+N279S+G303R+E390A+G475K, H1*+G7A+R180*+S181*+A185K+Y242F+E259G+N279S+G303R+E390A+G475K, H1*+G7A+R180*+S181*+A185D+Y242F+E259G+N279S+G303R+E390A+G475K, H1*+G7A+R180*+S181*+A185Q+Y242F+E259G+N279S+G303R+E390A+G475K, H1*+G7A+R180*+S181*+A185N+Y242F+E259G+N279S+G303R+E390A+G475K, H1*+G7A+R180*+S181*+A185R+Y242F+E259G+N279S+G303R+E390A+G475K, H1*+G7A+R180*+S181*+A185E+Y242F+E259G+N279S+G303R+E390A+G475K, H1*+G7A+R180*+S181*+E189P+Y242F+E259G+N279S+G303R+E390A+G475K, H1*+G7A+R180*+S181*+L205Y+Y242F+E259G+N279S+G303R+E390A+G475K, H1*+G7A+R180*+S181*+L205F+Y242F+E259G+N279S+G303R+E390A+G475K, H1*+G7A+R180*+S181*+Y242F+S243Q+E259G+N279S+G303R+E390A+G475K, H1*+G7A+R180*+S181*+Y242F+F244L+E259G+N279S+G303R+E390A+G475K, H1*+G7A+R180*+S181*+Y242F+F244I+E259G+N279S+G303R+E390A+G475K, H1*+G7A+R180*+S181*+Y242F+E259G+F266Y+N279S+G303R+E390A+G475K, H1*+G7A+R180*+S181*+N194F+Y242F+E259G+N279S+G303R+E390A+G475K, H1*+G7A+R180*+S181*+N196F+Y242F+E259G+N279S+G303R+E390A+G475K, H1*+G7A+R180*+S181*+Y242F+E259G+N279S+G303R+M322G+E390A+G475K, H1*+G7A+T40N+R180*+S181*+Y242F+E259G+N279S+G303R+E390A+G475K, H1*+G7S+Q98L+R180*+S181*+Y242F+E259G+N279S+G303R+E390A+G475K, H1*+G7S+R180*+S181*+Y242F+E259G+F261K+N279S+G303R+E390A+G475K, H1*+G7S+R180*+S181*+Y242F+E259G+N279S+G303R+Y362V+E390A+G475K, H1*+G7A+R180*+S181*+Y242F+E259G+N279S+G303R+Y362V+E390A+G475K, H1*+G7S+R180*+S181*+Y242F+E259G+F261S+N279S+G303R+E390A+G475K, H1*+G7A+R180*+S181*+Y242F+E259G+N279S+S286L+G303R+E390A+G475K, H1*+G7A+G110K+R180*+S181*+Y242F+E259G+N279S+G303R+E390A+G475K, H1*+G7A+W140S+R180*+S181*+Y242F+E259G+N279S+G303R+E390A+G475K, H1*+G7A+W167P+R180*+S181*+Y242F+E259G+N279S+G303R+E390A+G475K, H1*+G7A+R180*+S181*+Y242F+E259G+N279S+G303R+H323A+E390A+G475K, H1*+G7A+R180*+S181*+Y242F+E259G+N279S+G303R+H320T+E390A+G475K, H1*+G7A+R180*+S181*+Y242F+E259G+N279S+G303R+P321H+E390A+G475K, H1*+G7A+R180*+S181*+Y242F+E259G+N279S+G303R+M322Y+E390A+G475K, H1*+G7A+R180*+S181*+Y242F+E259G+N279S+G303R+Q360Y+E390A+G475K, H1*+G7A+Q98L+R180*+S181*+Y242F+E259G+N279S+G303R+E390A+G475K, H1*+G7A+R180*+S181*+Y242F+E259G+F261S+N279S+G303R+E390A+G475K, H1*+G7H+G110K+R180*+S181*+Y242F+E259G+N279S+G303R+E390A+G475K, H1*+G7A+Q98L+R180*+S181*+Y242F+E259G+N279S+G303R+E390A+G475K, H1*+G7A+R180*+S181*+Y242F+E259G+F261K+N279S+G303R+E390A+G475K and L63F+W140Y+L173F+R180*+S181*+N194F+L205Y+Y242F+E259G+N279S+H323W, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding to H1*+G7A+R180*+S181*+N194F+L205Y+Y242F+E259G+N279S+G303R+E390A+G475K, H1*+G7A+R180*+S181*+N194F+L205F+Y242F+E259G+N279S+G303R+E390A+G475K, H1*+G7A+R180*+S181*+N196F+L205Y+Y242F+E259G+N279S+G303R+E390A+G475K, H1*+G7A+R180*+S181*+E189P+Y242F+E259G+N276F+N279S+G303R+E390A+G475K, H1*+G7A+R180*+S181*+E189P+Y242F+E259G+N276Y+N279S+G303R+E390A+G475K, H1*+G7A+R180*+S181*+E189P+Y242F+E259G+N276W+N279S+G303R+E390A+G475K, H1*+G7A+R180*+S181*+E189P+Y242F+E259G+N279S+K280Y+G303R+E390A+G475K, H1*+G7A+R180*+S181*+E189P+Y242F+E259G+N279S+K280W+G303R+E390A+G475K, H1*+G7A+R180*+S181*+Y242F+E259G+N279S+G303R+M322G+Q360W+E390A+G475K, H1*+G7A+R180*+S181*+Y242F+E259G+N279S+G303R+E359W+Q360W+E390A+G475K, H1*+G7S+R180*+S181*+Y242F+E259G+F261S+N279S+G303R+Y362V+E390A+G475K, H1*+G7A+T40N+Q98L+R180*+S181*+Y242F+E259G+N279S+G303R+E390A+G475K, H1*+G7S+T40N+Q98L+R180*+S181*+Y242F+E259G+N279S+G303R+E390A+G475K, H1*+G7A+D123N+I176V+R180*+S181*+Y242F+E259G+N279S+G303R+E390A+G475K, H1*+G7A+R180*+S181*+Y242F+E259G+N279S+G303R+M322G+Q360W+E390A+G475K, H1*+G7A+T40N+Q98L+R180*+S181*+Y242F+E259G+N279S+G303R+E390A+G475K, H1*+G7A+R180*+S181*+Y242F+E259G+N279S+S286K+G303R+H323A+E390A+G475K, H1*+G7A+R180*+S181*+Y242F+E259G+N279S+G303R+H320T+H323A+E390A+G475K, H1*+G7A+R180*+S181*+Y242F+E259G+N279S+G303R+P321H+M322Y+E390A+G475K, H1*+G7A+R180*+S181*+Y242F+E259G+N279S+G303R+P321H+Q360Y+E390A+G475K, H1*+G7A+R180*+S181*+Y242F+E259G+N279S+G303R+M322Y+Q360Y+E390A+G475K, H1*+G7S+T40N+R180*+S181*+Y242F+E259G+N279S+G303R+Y362V+E390A+G475K, H1*+G7S+R180*+S181*+Y242F+E259G+F261S+N279S+G303R+Y362V+E390A+G475K, H1*+G7S+T40N+Q98L+R180*+S181*+Y242F+E259G+N279S+G303R+E390A+G475K, H1*+G7A+R180*+S181*+Y242F+E259G+F261S+N279S+G303R+Y362V+E390A+G475K, H1*+G7A+R180*+S181*+Y242F+E259G+N279S+S286L+G303R+H320T+E390A+G475K, H1*+G7H+Q98L+G110K+R180*+S181*+Y242F+E259G+N279S+G303R+E390A+G475K, H1*+G7A+Q98L+G110K+R180*+S181*+Y242F+E259G+N279S+G303R+E390A+G475K, H1*+G7H+Q98L+G110K+R180*+S181*+Y242F+E259G+N279S+G303R+E390A+G475K, H1*+G7A+Q98L+G110K+R180*+S181*+Y242F+E259G+N279S+G303R+E390A+G475K, H1*+G7A+R180*+S181*+Y242F+E259G+N279S+S286L+G303R+Y362V+E390A+G475K, H1*+G7A+R180*+S181*+Y242F+E259G+N279S+W283N+S286K+G303R+E390A+G475K, H1*+G7A+R180*+S181*+Y242F+E259G+N279S+W283N+G303R+H320T+E390A+G475K, H1*+G7A+R180*+S181*+Y242F+E259G+N279S+W283N+G303R+H323A+E390A+G475K, H1*+G7A+R180*+S181*+Y242F+E259G+N279S+S286K+G303R+H320T+E390A+G475K, H1*+G7A+R180*+S181*+Y242F+E259G+N279S+S286K+G303R+Y362V+E390A+G475K, H1*+G7A+R180*+S181*+Y242F+E259G+N279S+G303R+M322G+E359W+E390A+G475K and W140Y+L173F+R180*+S181*+N194F+L205Y+N225S+Y242F+E259G+N279S+G303R+H323A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding to H1*+G7A+R180*+S181*+E189P+L205F+Y242F+E259G+N276F+N279S+G303R+E390A+G475K, H1*+G7A+R180*+S181*+E189P+L205F+Y242F+E259G+N276Y+N279S+G303R+E390A+G475K, H1*+G7A+R180*+S181*+E189P+L205F+Y242F+E259G+N276W+N279S+G303R+E390A+G475K, H1*+G7A+R180*+S181*+E189P+L205F+Y242F+E259G+N279S+K280F+G303R+E390A+G475K, H1*+G7A+R180*+S181*+E189P+L205F+Y242F+E259G+N279S+K280Y+G303R+E390A+G475K, H1*+G7A+R180*+S181*+E189P+L205F+Y242F+E259G+N279S+K280W+G303R+E390A+G475K, H1*+G7A+R180*+S181*+E189P+L205Y+Y242F+E259G+N276F+N279S+G303R+E390A+G475K, H1*+G7A+R180*+S181*+E189P+L205Y+Y242F+E259G+N276Y+N279S+G303R+E390A+G475K, H1*+G7A+R180*+S181*+E189P+L205Y+Y242F+E259G+N276W+N279S+G303R+E390A+G475K, H1*+G7A+R180*+S181*+E189P+L205Y+Y242F+E259G+N279S+K280F+G303R+E390A+G475K, H1*+G7A+R180*+S181*+E189P+L205Y+Y242F+E259G+N279S+K280Y+G303R+E390A+G475K, H1*+G7A+R180*+S181*+E189P+L205Y+Y242F+E259G+N279S+K280W+G303R+E390A+G475K, H1*+G7A+G113S+F116W+D123N+S181*+T182*+Y242F+E259G+N279S+G303R+E390A+G475K, H1*+G7S+T40N+Q98L+R180*+S181*+Y242F+E259G+N279S+G303R+Y362V+E390A+G475K, H1*+G7A+R180*+S181*+Y242F+E259G+N279S+G303R+M322G+E359W+Q360W+E390A+G475K, H1*+G7A+R180*+S181*+Y242F+E259G+F261S+N279S+S286K+G303R+Y362V+E390A+G475K, H1*+G7A+R180*+S181*+Y242F+E259G+N279S+G303R+P321H+M322Y+Q360Y+E390A+G475K, H1*+G7A+G113S+F116W+D123N+R180*+S181*+Y242F+E259G+N279S+G303R+E390A+G475K, H1*+G7S+T40N+Q98L+R180*+S181*+Y242F+E259G+N279S+G303R+H320T+E390A+G475K, H1*+G7S+T40N+Q98L+R180*+S181*+Y242F+E259G+N279S+G303R+Y362V+E390A+G475K, H1*+G7A+1137F+R180*+S181*+Y242F+E259G+F261S+N279S+G303R+Y362V+E390A+G475K, H1*+G7A+R180*+S181*+Y242F+E259G+F261S+N279S+S286K+G303R+Y362V+E390A+G475K, H1*+G7A+R180*+S181*+Y242F+E259G+N279S+W283N+G303R+H320T+H323A+E390A+G475K, H1*+G7A+Q98L+R180*+S181*+Y242F+E259G+F261K+N279S+G303R+Y362V+E390A+G475K, H1*+G7H+Q98L+R180*+S181*+Y242F+E259G+N279S+G303R+A383D+E390A+D405G+G475K, H1*+G7A+Q98L+G110K+R180*+S181*+Y242F+T256A+E259G+N279S+G303R+E390A+G475K, H1*+G7A+Q98L+G110K+R180*+S181*+Y242F+E259G+N279S+G303R+A383D+E390A+G475K, H1*+G7A+W140Y+R180*+S181*+N194F+L205Y+Y242F+E259G+N279S+G303R+E390A+G476A, H1*+G7A+W140Y+R180*+S181*+N194F+L205Y+Y242F+E259G+N279S+G303R+N394W+G475K, H1*+G7A+W140Y+R180*+S181*+N194F+L205Y+Y242F+E259G+N279S+G303R+E390A+N474L, H1*+G7A+W140Y+R180*+S181*+N194F+L205Y+Y242F+E259G+N279S+S302T+E390A+G475K, H1*+G7A+W140Y+R180*+S181*+N194F+L205Y+Y242F+E259G+N279S+G303R+E390A+G475E, H1*+G7A+W140Y+R180*+S181*+N194F+L205Y+Y242F+E259G+N279S+G303R+E390A+N474F, H1*+G7A+W140Y+R180*+S181*+N194F+L205Y+Y242F+E259G+N279S+S302Y+E390A+G475K, H1*+G7A+W140Y+R180*+S181*+N194F+L205Y+Y242F+T256D+N279S+G303R+E390A+G475K, H1*+G7A+R37A+W140Y+R180*+S181*+N194F+L205Y+Y242F+E259G+N279S+G303R+G475K, H1*+W140Y+R180*+S181*+N194F+L205Y+Y242F+E259G+N279S+G303R+Q360Y+E390A+G475K, H1*+G7A+R37N+W140Y+R180*+S181*+N194F+L205Y+Y242F+E259G+N279S+G303R+G475K, H1*+W140Y+R180*+S181*+N194F+L205Y+Y242F+E259G+N279S+G303R+E390A+R414M+G475K, H1*+W140Y+R180*+S181*+N194F+L205Y+Y242F+E259G+F261P+N279S+G303R+E390A+G475K, H1*+W140Y+R180*+S181*+N194F+L205Y+Y242F+E259G+N279S+G303R+H323W+E390A+G475K, H1*+W140Y+R180*+S181*+N194F+L205Y+Y242F+E259G+N279S+G303R+P321Y+E390A+G475K, H1*+W140Y+R180*+S181*+N194F+L205Y+Y242F+E259G+N279S+G303R+Q360M+E390A+G475K, H1*+G7A+W140Y+R180*+S181*+Y242F+S243Q+E259G+N279S+K280Y+G303R+E390A+G475K, H1*+G7A+W140Y+R180*+S181*+Y242F+E259G+F266Y+N279S+K280Y+G303R+E390A+G475K and H1*+G7A+W140Y+R180*+S181*+T182D+A185Q+Y242F+E259G+N279S+G303R+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding: H1*+G7A+R180*+S181*+Y242F+E259G+N279S+W283N+S286K+G303R+H320T+H323A+E390A+G475K, H1*+G7A+W140Y+R180*+S181*+T182D+N194F+L205Y+Y242F+E259G+N279S+G303R+E390A+G475K, H1*+G7A+W140Y+R180*+S181*+T182D+N194F+L205V+Y242F+E259G+N279S+G303R+E390A+G475K, H1*+G7A+W140Y+R180*+S181*+T182S+N194F+L205Y+Y242F+E259G+N279S+G303R+E390A+G475K, H1*+G7A+W140Y+R180*+S181*+T182S+N194F+L205V+Y242F+E259G+N279S+G303R+E390A+G475K, H1*+G7A+D123N+W140Y+R180*+S181*+N194F+L205V+Y242F+E259G+N279S+G303R+E390A+G475K, H1*+G7A+D123N+W140Y+R180*+S181*+N194F+L205Y+Y242F+E259G+N279S+G303R+E390A+G475K, H1*+G7A+Q98L+G110K+P146S+R180*+S181*+Y242F+E259G+N279S+G303R+A383D+E390A+G475K, H1*+N3S+G7A+Q98L+G110K+R180*+S181*+Y242F+V252+E259G+N279S+G303R+E390A+G475K, H1*+G7A+W140Y+R180*+S181*+N194F+L205V+Y242F+E259G+F266Y+N279S+G303R+E390A+G475K, H1*+G7A+W140Y+R180*+S181*+A185Q+N194F+L205V+Y242F+E259G+N279S+G303R+E390A+G475K, H1*+G7A+W140Y+R180*+S181*+A185Q+N194F+L205Y+Y242F+E259G+N279S+G303R+E390A+G475K, H1*+G7A+W140Y+R180*+S181*+E189P+N194F+Y242F+E259G+N279S+K280Y+G303R+E390A+G475K, H1*+G7A+W140Y+R180*+S181*+E189P+N194F+Y242F+E259G+N279S+K280Y+G303R+E390A+G475K, H1*+G7A+W140Y+R180*+S181*+N194F+L205Y+Y242F+E259G+F266Y+N279S+G303R+E390A+G475K, H1*+G7A+W140Y+R180*+S181*+N194F+L205Y+Y242F+E259G+N279S+S302T+G303R+E390A+G475K, H1*+G7A+W140Y+R180*+S181*+N194F+L205Y+Y242F+E259G+N279S+G303R+E390A+N474F+G475K, H1*+G7A+W140Y+R180*+S181*+N194F+L205Y+Y242F+E259G+N279S+G303R+E390A+N422V+G475K, H1*+G7A+W140Y+R180*+S181*+N194F+L205Y+Y242F+E259G+F261P+N279S+G303R+E390A+G475K, H1*+G7A+W140Y+R180*+S181*+N194F+L205Y+Y242F+E259G+N279S+G303R+E390A+N470A+G475K, H1*+G7A+W140Y+R180*+S181*+N194F+L205Y+Y242L+E259G+N279S+G303R+W346N+E390A+G475K, H1*+G7A+W140Y+R180*+S181*+N194F+L205Y+Y242F+E259G+N279S+G303R+E390A+H420P+G475K, H1*+G7A+R26Y+W140Y+R180*+S181*+N194F+L205Y+Y242F+E259G+N279S+G303R+E390A+G475K, H1*+G7A+W140Y+R180*+S181*+N194F+L205Y+Y242F+E259G+N279S+G303R+Y371S+E390A+G475K, H1*+G7A+W140Y+R180*+S181*+T182D+A185Q+Y242F+E259G+N279S+K280Y+G303R+E390A+G475K, H1*+G7A+W140Y+R180*+S181*+A185Q+E189P+Y242F+E259G+N279S+K280Y+G303R+E390A+G475K, H1*+G7A+W140Y+R180*+S181*+E189P+Y242F+S243Q+E259G+N279S+K280Y+G303R+E390A+G475K, H1*+G7A+W140Y+R180*+S181*+T182D+Y242F+S243Q+E259G+N279S+K280Y+G303R+E390A+G475K, H1*+G7A+W140Y+R180*+S181*+Y242F+S243Q+E259G+F266Y+N279S+K280Y+G303R+E390A+G475K, H1*+G7A+A41D+W140Y+R180*+S181*+N194F+L205Y+Y242F+E259G+N279S+G303R+E390A+G475K and H1*+G7A+T40G+W140Y+R180*+S181*+N194F+L205Y+Y242F+E259G+N279S+G303R+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding: H1*+G7A+F116L+D123N+N126D+T131 I+S181*+T182*+F202Y+Y242F+E259G+N279S+G303R+E390A+G475K, H1*+G7A+F116L+D123N+N126D+T131I+R180*+S181*+F202Y+Y242F+E259G+N279S+G303R+E390A+G475K, H1*+G7A+D118T+W140Y+R180*+S181*+T182D+N194F+L205Y+Y242F+E259G+N279S+G303R+E390A+G475K, H1*+G7A+F116N+W140Y+R180*+S181*+T182D+N194F+L205Y+Y242F+E259G+N279S+G303R+E390A+G475K, H1*+G7A+W140Y+R180*+S181*+E189P+N194F+L205Y+Y242F+E259G+N279S+K280Y+G303R+E390A+G475K, H1*+G7A+W140Y+R180*+S181*+T182D+E189P+N194F+L205Y+Y242F+E259G+N279S+G303R+E390A+G475K, H1*+G7A+T134D+W140Y+R180*+S181*+E189P+N194F+L205V+Y242F+E259G+N279S+G303R+E390A+G475K, H1*+G7A+D118T+W140Y+R180*+S181*+T182S+N194F+L205Y+Y242F+E259G+N279S+G303R+E390A+G475K, H1*+G7A+F116N+W140Y+R180*+S181*+T182S+N194F+L205Y+Y242F+E259G+N279S+G303R+E390A+G475K, H1*+G7A+W140Y+R180*+S181*+T182S+E189P+N194F+L205Y+Y242F+E259G+N279S+G303R+E390A+G475K, H1*+G7A+W140Y+R180*+S181*+T182S+E189P+N194F+L205V+Y242F+E259G+N279S+G303R+E390A+G475K, H1*+G7A+D123N+W140Y+I176V+R180*+S181*+N194F+L205V+Y242F+E259G+N279S+G303R+E390A+G475K, H1*+G7A+D123N+W140Y+I176V+R180*+S181*+N194F+L205Y+Y242F+E259G+N279S+G303R+E390A+G475K, H1*+G7A+Q98L+G110K+S132D+Y135E+R180*+S181*+Y242F+E259G+N279S+G303R+T315A+E390A+G475K, H1*+A60V+Q98L+W140Y+R180*+S181*+N194F+L205Y+Y242F+E259G+N279S+G303R+A383D+E390A+G475K, H1*+G7A+W140Y+T151A+R180*+S181*+N194F+L205Y+Y242F+K258V+E259V+N279S+G303R+E390A+G475K, H1*+N3S+G7A+N94D+Q98L+G110K+R180*+S181*+Y242F+V252I+E259G+N279S+G303R+E390A+G475K, H1*+G7A+W140Y+T151A+R180*+S181*+N194F+L205Y+Y242F+K258V+E259V+N279S+G303R+E390A+G475K, H1*+G7A+F116N+W140Y+R180*+S181*+N194F+L205Y+Y242F+E259G+F266Y+N279S+G303R+E390A+G475K, H1*+G7A+F116N+W140Y+R180*+S181*+A185Q+N194F+L205Y+Y242F+E259G+N279S+G303R+E390A+G475K, H1*+G7A+D118T+W140Y+R180*+S181*+E189P+N194F+Y242F+E259G+N279S+K280Y+G303R+E390A+G475K, H1*+G7A+D118T+W140Y+R180*+S181*+N194F+L205Y+Y242F+E259G+F266Y+N279S+G303R+E390A+G475K, H1*+G7A+D118T+W140Y+R180*+S181*+A185Q+N194F+L205Y+Y242F+E259G+N279S+G303R+E390A+G475K, H1*+G7A+W140Y+R180*+S181*+E189P+N194F+L205V+Y242F+E259G+N279S+K280Y+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+T182S+E189P+N194F+Y242F+E259G+N279S+K280Y+G303R+E390A+G475K, H1*+G7A+W140Y+R180*+S181*+T182D+N194F+L205V+Y242F+E259G+F266Y+N279S+G303R+E390A+G475K, H1*+G7A+W140Y+R180*+S181*+A185Q+N194F+L205V+Y242F+E259G+F266Y+N279S+G303R+E390A+G475K, H1*+G7A+W140Y+R180*+S181*+T182D+A185Q+N194F+L205V+Y242F+E259G+N279S+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+T182S+E189P+N194F+Y242F+E259G+N279S+K280Y+G303R+E390A+G475K, H1*+G7A+W140Y+R180*+S181*+T182D+N194F+L205Y+Y242F+E259G+F266Y+N279S+G303R+E390A+G475K, H1*+G7A+W140Y+R180*+S181*+A185Q+N194F+L205Y+Y242F+E259G+F266Y+N279S+G303R+E390A+G475K, H1*+G7A+W140Y+R180*+S181*+T182D+A185Q+N194F+L205Y+Y242F+E259G+N279S+G303R+E390A+G475K, H1*+G7A+F116N+W140Y+R180*+S181*+E189P+N194F+Y242F+E259G+N279S+K280Y+G303R+E390A+G475K, H1*+G7A+W140Y+R180*+S181*+T182D+Y242F+S243Q+E259G+F266Y+N279S+K280Y+G303R+E390A+G475K, H1*+G7A+W140Y+R180*+S181*+T182D+E189P+Y242F+S243Q+E259G+N279S+K280Y+G303R+E390A+G475K, H1*+G7A+W140Y+R180*+S181*+A185Q+Y242F+E259G+F266Y+N279S+K280Y+G303R+E390A+T427L+G475K, H1*+G7A+W140Y+R180*+S181*+A185Q+Y242F+S243Q+E259G+F266Y+N279S+K280Y+G303R+E390A+G475K, H1*+G7A+W140Y+R180*+S181*+T182D+A185Q+Y242F+S243Q+E259G+F266Y+N279S+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+A185Q+N194F+Y242F+S243Q+E259G+N279S+K280Y+G303R+E390A+G475K, H1*+G7A+W140Y+R180*+S181*+T182D+N194F+Y242F+S243Q+E259G+N279S+K280Y+G303R+E390A+G475K, H1*+G7A+W140Y+R180*+S181*+A185Q+E189P+N194F+Y242F+E259G+N279S+K280Y+G303R+E390A+G475K, H1*+G7A+W140Y+R180*+S181*+E189P+L205Y+Y242F+S243Q+E259G+N279S+K280Y+G303R+E390A+G475K, H1*+G7A+W140Y+R180*+S181*+T182D+A185Q+N194F+Y242F+S243Q+E259G+N279S+G303R+E390A+G475K, H1*+G7A+W140Y+R180*+S181*+T182D+N194F+Y242F+S243Q+E259G+F266Y+N279S+G303R+E390A+G475K, H1*+G7A+W140Y+R180*+S181*+E189P+N194F+Y242F+E259G+F266Y+N279S+K280Y+G303R+E390A+G475K, H1*+G7A+W140Y+R180*+S181*+T182D+A185Q+N194F+Y242F+E259G+F266Y+N279S+G303R+E390A+G475K, H1*+G7A+W140Y+R180*+S181*+A185Q+N194F+Y242F+E259G+F266Y+N279S+K280Y+G303R+E390A+G475K, H1*+G7A+W140Y+R180*+S181*+A185Q+E189P+L205Y+Y242F+E259G+N279S+K280Y+G303R+E390A+G475K, H1*+G7A+W140Y+R180*+S181*+E189P+N194F+Y242F+E259G+F266Y+N279S+K280Y+S302T+E390A+G475K, H1*+G7A+W140Y+R180*+S181*+E189P+N194F+Y242F+E259G+M260D+N279S+K280Y+S302T+E390A+G475K, H1*+G7A+W140Y+R180*+S181*+E189P+N194F+Y242F+E259G+F266Y+N279S+K280Y+G303R+E390A+G476A and

H1*+G7A+W140Y+R180*+S181*+E189P+N194F+Y242F+E259G+M260W+N279S+K280Y+G303R+E390A+G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding: H1*+G7A+D118T+W140Y+R180*+S181*+E189P+N194F+L205Y+Y242F+E259G+N279S+K280Y+G303R+E390A+G475K, H1*+G7A+F116N+W140Y+R180*+S181*+E189P+N194F+L205Y+Y242F+E259G+N279S+K280Y+G303R+E390A+G475K, H1*+G7A+F116N+W140Y+R180*+S181*+T182D+E189P+N194F+L205Y+Y242F+E259G+N279S+G303R+E390A+G475K, H1*+G7A+G113S+F116W+D123N+W140Y+R180*+S181*+N194F+L205V+Y242F+E259G+N279S+G303R+E390A+G475K, H1*+G7A+N106D+A109G+W140Y+R180*+S181*+N194F+F202Y+L205V+Y242F+E259G+N279S+G303R+E390A+G475K, H1*+G7A+G113S+F116W+D123N+W140Y+R180*+S181*+N194F+L205Y+Y242F+E259G+N279S+G303R+E390A+G475K, H1*+G7A+F116N+W140Y+R180*+S181*+T182D+E189P+N194F+Y242F+E259G+N279S+K280Y+G303R+E390A+G475K, H1*+G7A+F116N+W140Y+R180*+S181*+T182S+E189P+N194F+Y242F+E259G+N279S+K280Y+G303R+E390A+G475K, H1*+G7A+F116N+W140Y+R180*+S181*+T182D+N194F+L205Y+Y242F+E259G+F266Y+N279S+G303R+E390A+G475K, H1*+G7A+F116N+W140Y+R180*+S181*+A185Q+N194F+L205Y+Y242F+E259G+F266Y+N279S+G303R+E390A+G475K, H1*+G7A+F116N+W140Y+R180*+S181*+T182D+A185Q+N194F+L205Y+Y242F+E259G+N279S+G303R+E390A+G475K, H1*+G7A+D118T+W140Y+R180*+S181*+T182D+N194F+L205Y+Y242F+E259G+F266Y+N279S+G303R+E390A+G475K, H1*+G7A+D118T+W140Y+R180*+S181*+A185Q+N194F+L205Y+Y242F+E259G+F266Y+N279S+G303R+E390A+G475K, H1*+G7A+D118T+W140Y+R180*+S181*+T182D+A185Q+N194F+L205Y+Y242F+E259G+N279S+G303R+E390A+G475K, H1*+G7A+W140Y+R180*+S181*+T182D+E189P+N194F+L205V+Y242F+E259G+N279S+K280Y+G303R+E390A+G475K, H1*+G7A+W140Y+R180*+S181*+T182D+A185Q+N194F+L205V+Y242F+E259G+F266Y+N279S+G303R+E390A+G475K, H1*+G7A+W140Y+R180*+S181*+T182D+E189P+N194F+L205Y+Y242F+E259G+N279S+K280Y+G303R+E390A+G475K, H1*+G7A+W140Y+R180*+S181*+T182S+E189P+N194F+L205Y+Y242F+E259G+N279S+K280Y+G303R+E390A+G475K, H1*+G7A+W140Y+R180*+S181*+T182D+A185Q+N194F+L205Y+Y242F+E259G+F266Y+N279S+G303R+E390A+G475K, H1*+G7A+D118T+W140Y+R180*+S181*+T182D+E189P+N194F+Y242F+E259G+N279S+K280Y+G303R+E390A+G475K, H1*+G7A+D118T+W140Y+R180*+S181*+T182S+E189P+N194F+Y242F+E259G+N279S+K280Y+G303R+E390A+G475K, H1*+G7A+W140Y+R180*+S181*+T182D+A185Q+N194F+Y242F+S243Q+E259G+N279S+K280Y+G303R+E390A+G475K, H1*+G7A+W140Y+R180*+S181*+A185Q+N194F+Y242F+S243Q+E259G+F266Y+N279S+K280Y+G303R+E390A+G475K, H1*+G7A+W140Y+R180*+S181*+T182D+E189P+L205Y+Y242F+E259G+F266Y+N279S+K280Y+G303R+E390A+G475K, H1*+G7A+W140Y+R180*+S181*+A185Q+E189P+N194F+Y242F+E259G+F266Y+N279S+K280Y+G303R+E390A+G475K, H1*+G7A+W140Y+R180*+S181*+T182D+E189P+N194F+Y242F+S243Q+E259G+N279S+K280Y+G303R+E390A+G475K, H1*+D118T+W140Y+R180*+S181*+T182S+E189P+N194F+L205Y+Y242F+E259G+N279S+K280Y+G303R+E390A+G475K, H1*+G7A+W140Y+R180*+S181*+T182S+E189P+N194F+L205Y+Y242F+E259G+N279S+K280Y+G303R+E390A+G475K, H1*+G7A+D118T+R180*+S181*+T182S+E189P+N194F+L205Y+Y242F+E259G+N279S+K280Y+G303R+E390A+G475K, H1*+G7A+D118T+W140Y+R180*+S181*+E189P+N194F+L205Y+Y242F+E259G+N279S+K280Y+G303R+E390A+G475K, H1*+G7A+D118T+W140Y+R180*+S181*+T182S+N194F+L205Y+Y242F+E259G+N279S+K280Y+G303R+E390A+G475K, H1*+G7A+D118T+W140Y+R180*+S181*+T182S+E189P+L205Y+Y242F+E259G+N279S+K280Y+G303R+E390A+G475K, H1*+G7A+D118T+W140Y+R180*+S181*+T182S+E189P+N194F+Y242F+E259G+N279S+K280Y+G303R+E390A+G475K, H1*+G7A+D118T+W140Y+R180*+S181*+T182S+E189P+N194F+L205Y+E259G+N279S+K280Y+G303R+E390A+G475K, H1*+G7A+D118T+W140Y+R180*+S181*+T182S+E189P+N194F+L205Y+Y242F+N279S+K280Y+G303R+E390A+G475K, H1*+G7A+D118T+W140Y+R180*+S181*+T182S+E189P+N194F+L205Y+Y242F+E259G+K280Y+G303R+E390A+G475K, H1*+G7A+D118T+W140Y+R180*+S181*+T182S+E189P+N194F+L205Y+Y242F+E259G+N279S+G303R+E390A+G475K, H1*+G7A+D118T+W140Y+R180*+S181*+T182S+E189P+N194F+L205Y+Y242F+E259G+N279S+K280Y+E390A+G475K, H1*+G7A+D118T+W140Y+R180*+S181*+T182S+E189P+N194F+L205Y+Y242F+E259G+N279S+K280Y+G303R+G475K, H1*+G7A+D118T+W140Y+R180*+S181*+T182S+E189P+N194F+L205Y+Y242F+E259G+N279S+K280Y+G303R+E390A, H1*+G7A+D118T+W140Y+R180*+S181*+T182S+E189P+N194F+Y242F+E259G+N279S+K280Y+S302T+E390A+G475K, H1*+G7A+D118T+W140Y+R180*+S181*+T182D+E189P+N194F+Y242F+E259G+N279S+K280Y+G303R+E390A+G476A, H1*+G7A+R37N+W140Y+R180*+S181*+E189P+N194F+Y242F+E259G+F266Y+N279S+K280Y+S302Y+E390A+G475K, H1*+G7A+D118T+W140Y+R180*+S181*+T182S+E189P+N194F+T224Y+Y242F+N279S+K280Y+G303R+E390A+G476A, H1*+G7A+D118T+W140Y+R180*+S181*+T182D+E189P+N194F+T224Y+Y242F+N279S+K280Y+G303R+E390A+G476A, H1*+G7A+D118T+W140Y+R180*+S181*+T182D+A185Q+Y242F+S243Q+E259G+F266Y+N279S+G303R+E390A+G475K, H1*+G7A+D118T+W140Y+R180*+S181*+T182D+N194F+Y242F+S243Q+E259G+F266Y+N279S+G303R+E390A+G475K, H1*+G7A+D118T+W140Y+R180*+S181*+T182D+E189P+N194F+T224Y+Y242F+N279S+K280Y+G303R+E390A+G476A, H1*+G7A+D118T+W140Y+R180*+S181*+T182S+E189P+N194F+T224Y+Y242F+N279S+K280Y+G303R+E390A+G476A, H1*+G7A+D118T+W140Y+R180*+S181*+T182D+A185Q+N194F+Y242F+E259G+N279S+K280Y+S302T+E390A+G475K, H1*+G7A+W140Y+R180*+S181*+E189P+N194F+Y242F+E259G+M260D+N279S+K280Y+S302Y+E390A+N470A+G475K,

H1*+G7A+S132L+W140Y+R180*+S181*+E189P+N194F+Y242F+E259G+M260D+N279S+K280Y+G303R+E390A+G475K, and T40N+F116L+D123N+N126D+N128Y+T131I+F155W+R180*+S181*+T182D+E189P+F202Y+K457N+P458S+G475K+G476E, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding to H1*+G7A+G113A+F116W+D123N+T131V+Q136K+E169Q+K172Q+R180*+S181*+Y242F+E259G+N279S+G303R+E390A+G475K, H1*+G7A+F116N+W140Y+R180*+S181*+T182S+E189P+N194F+L205Y+Y242F+E259G+N279S+K280Y+G303R+E390A+G475K, H1*+N3S+G7A+N94D+Q98L+W140Y+R180*+S181*+N194F+L205Y+Y242F+V252+E259G+N279S+G303R+E390A+G475K, H1*+G7A+F116N+W140Y+R180*+S181*+T182D+E189P+N194F+L205Y+Y242F+E259G+N279S+K280Y+G303R+E390A+G475K, H1*+G7A+F116N+W140Y+R180*+S181*+T182D+A185Q+N194F+L205Y+Y242F+E259G+F266Y+N279S+G303R+E390A+G475K, H1*+G7A+D118T+W140Y+R180*+S181*+T182S+E189P+N194F+L205Y+Y242F+E259G+N279S+K280Y+G303R+E390A+G475K, H1*+G7A+D118T+W140Y+R180*+S181*+T182D+A185Q+N194F+L205Y+Y242F+E259G+F266Y+N279S+G303R+E390A+G475K, H1*+G7A+D118T+W140Y+R180*+S181*+T182D+E189P+N194F+L205Y+Y242F+E259G+N279S+K280Y+G303R+E390A+G475K, H1*+G7A+W140Y+R180*+S181*+T182D+A185Q+E189P+N194F+Y242F+S243Q+E259G+N279S+K280Y+G303R+E390A+G475K, H1*+G7A+W140Y+R180*+S181*+A185Q+E189P+N194F+Y242F+S243Q+E259G+F266Y+N279S+K280Y+G303R+E390A+G475K, H1*+G7A+W140Y+R180*+S181*+T182D+A185Q+E189P+N194F+Y242F+E259G+F266Y+N279S+K280Y+G303R+E390A+G475K, H1*+G7A+W140Y+R180*+S181*+A185Q+E189P+N194F+L205Y+Y242F+S243Q+E259G+N279S+K280Y+G303R+E390A+G475K, H1*+G7A+W140Y+R180*+S181*+T182D+E189P+L205Y+Y242F+S243Q+E259G+N279S+K280Y+G303R+E390A+I409L+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+S302Y+N394W+G475K, H1*+G7A+D118T+W140Y+R180*+S181*+T182S+E189P+N194F+L205Y+Y242F+E259G+N279S+K280Y+S302T+N394W+G475K, H1*+G7A+D118T+W140Y+R180*+S181*+T182S+E189P+N194F+L205Y+Y242F+E259G+N279S+K280Y+S302T+E390A+G476A, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302Y+E390A+G475K H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302T+E390A+G475K, H1*+G7A+R37N+D118T+W140Y+R180*+S181*+T182S+E189P+N194F+L205Y+Y242F+E259+N279S+K280Y+S302Y+E390A+G476H, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+G303R+E390A+N474L, H1*+G7A+D118T+W140Y+R180*+S181*+T182S+E189P+N194F+L205Y+T224Y+Y242F+N279S+K280Y+G303R+E390A+G476A H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302Y+E390A+N474F, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+G475K, H1*+G7A+D118T+W140Y+R180*+S181*+T182S+E189P+N194F+L205V+Y242F+E259G+N279S+K280Y+G303R+E390A+G475K, H1*+G7A+D118T+W140Y+R180*+S181*+T182S+E189P+N194F+L205N+Y242F+E259G+N279S+K280Y+G303R+E390A+G475K, H1*+G7A+D118T+W140Y+R180*+S181*+T182S+E189P+N194F+L205Q+Y242F+E259G+N279S+K280Y+G303R+E390A+G475K, H1*+G7A+D118T+W140Y+R180*+S181*+A185Q+E189P+N194F+L205Y+Y242F+E259G+N279S+K280Y+G303R+E390A+G475K, H1*+G7A+D118T+W140Y+R180*+S181*+T182S+A185Q+E189P+Y242F+E259G+F266Y+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+N474F, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+N394D+G475K, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302T+N394W+G475K, H1*+G7A+D118T+W140Y+R180*+S181*+T182S+E189P+N194F+L205Y+Y242F+E259G+N279S+K280Y+S302T+N394D+G475K, H1*+G7A+D118T+W140Y+R180*+S181*+T182S+A185D+E189P+Y242F+E259G+F266Y+N279S+K280Y+G303R+E390A+G475K, H1*+G7A+D118T+W140Y+R180Q+S181*+T182*+A185Q+E189P+Y242F+E259G+F266Y+N279S+K280Y+G303R+E390A+G475K, H1*+G7A+D118T+W140Y+R180*+S181*+T182S+E189P+N194F+Y242F+E259G+M260D+N279S+K280Y+S302T+E390A+G475K, H1*+G7A+R37N+D118T+W140Y+R180*+S181*+T182S+E189P+N194F+Y242F+E259G+N279S+K280Y+S302Y+E390A+G475K, H1*+G7A+D118T+W140Y+R180*+S181*+T182S+E189P+N194F+Y242F+E259G+M260W+N279S+K280Y+G303R+E390A+G476A, H1*+G7A+D118T+W140Y+R180*+S181*+T182D+E189P+N194F+Y242F+E259G+M260D+N279S+K280Y+S302T+E390A+G475K, H1*+G7A+R37N+D118T+W140Y+R180*+S181*+T182D+E189P+N194F+Y242F+E259G+N279S+K280Y+S302Y+E390A+G475K, H1*+G7A+D118T+W140Y+R180*+S181*+T182S+A185Q+E189P+N194F+Y242F+E259G+N279S+K280Y+G303R+E390A+G476A, H1*+G7A+D118T+W140Y+R180*+S181*+T182S+E189P+N194F+T224Y+Y242F+F266Y+N279S+K280Y+G303R+E390A+G476A, H1*+G7A+D118T+W140Y+R180*+S181*+T182S+E189P+N194F+Y242F+E259G+F266Y+N279S+K280Y+S302T+E390A+G475K, H1*+G7A+D118T+W140Y+R180*+S181*+T182S+E189P+N194F+Y242F+E259G+F266Y+N279S+K280Y+G303R+E390A+G476A, H1*+G7A+D118T+W140Y+R180*+S181*+T182D+E189P+N194F+Y242F+E259G+F266Y+N279S+K280Y+S302T+E390A+G475K, H1*+N3D+G7A+W140Y+R180*+S181*+E189P+N194F+Y242F+E259G+F266Y+N279S+K280Y+S302T+E390A+H420A+G475K, H1*+G7A+D118T+W140Y+R180*+S181*+T182D+E189P+N194F+Y242F+E259G+M260W+N279S+K280Y+G303R+E390A+G476A, H1*+G7A+D118T+W140Y+R180*+S181*+T182S+A185Q+E189P+N194F+T224Y+Y242F+N279S+K280Y+G303R+E390A+G476A, H1*+G7A+D118T+W140Y+R180*+S181*+T182S+A185Q+E189P+N194F+Y242F+E259G+N279S+K280Y+S302T+E390A+G475K, H1*+G7A+D118T+W140Y+R180*+S181*+T182D+E189P+N194F+Y242F+E259G+F266Y+N279S+K280Y+G303R+E390A+G476A, H1*+G7A+D118T+W140Y+R180*+S181*+T182D+A185Q+E189P+N194F+T224Y+Y242F+N279S+K280Y+G303R+E390A+G476A and H1*+G7A+D118T+W140Y+R180*+S181*+T182S+A185Q+E189P+N194F+Y242F+E259G+N279S+K280Y+S302T+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding to H1*+G7A+F116L+D123N+N126D+T131I+W140Y+R180*+S181*+N194F+F202Y+L205V+Y242F+E259G+N279S+G303R+E390A+G475K, H1*+G7A+F116L+D123N+N126D+T131I+W140Y+R180*+S181*+N194F+F202Y+L205Y+Y242F+E259G+N279S+G303R+E390A+G475K, H1*+G7A+W140Y+R180*+S181*+T182D+A185Q+E189P+N194F+L205Y+Y242F+S243Q+E259G+N279S+K280Y+G303R+E390A+G475K, H1*+G7A+W140Y+R180*+S181*+A185Q+E189P+N194F+L205Y+Y242F+S243Q+E259G+F266Y+N279S+K280Y+G303R+E390A+G475K, H1*+G7A+W140Y+R180*+S181*+T182D+A185Q+E189P+N194F+L205Y+Y242F+E259G+F266Y+N279S+K280Y+G303R+E390A+G475K, H1*+N3D+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+H420A+G475K, H1*+G7A+D118T+W140Y+R180*+S181*+T182S+E189P+N194F+L205Y+Y242F+E259G+N279S+K280Y+S302Y+E390A+H420A+G475K, H1*+N3D+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+H420A+G475K, H1*+G7A+D118T+W140Y+R180*+S181*+T182S+E189P+N194F+L205Y+Y242F+E259G+N279S+K280Y+G303R+E390A+H420A+G476A, H1*+N3D+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+H420A+N474L, H1*+G7A+D118T+W140Y+R180*+S181*+T182S+E189P+N194F+L205Y+Y242F+E259G+N279S+K280Y+G303R+N394W+H420P+N474L, H1*+G7A+G73L+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302Y+E390A+G475K, H1*+G7A+R37N+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302T+E390A+G476H, H1*+G7A+D118T+W140Y+R180*+S181*+T182S+E189P+N194F+L205Y+Y242F+E259G+N279S+K280Y+G303R+N394D+H420A+G475K, H1*+G7A+D118T+W140Y+R180*+S181*+T182S+E189P+N194F+L205Y+Y242F+E259G+N279S+K280Y+G303R+E390A+H420P+G475K, N3G+G7A+D118T+W140Y+R180*+S181*+T182S+E189P+N194F+L205Y+Y242F+E259G+N279S+K280Y+G303R+E390A+H420A+N474L, H1*+G7A+D118T+Y135E+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+G303R+N394W+H420A+G475K, H1*+G7A+V103I+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+M260D+N279S+K280Y+S302Y+E390A+G475K, H1*+G7A+D118T+W140Y+P146H+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+F261P+N279S+K280Y+G303R+E390A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+Q98L+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+G303R+P321H+E390A+G475K, H1*+G7A+D118T+W140Y+R180*+S181*+T182S+E189P+N194F+L205Y+Y242F+E259G+N279S+K280Y+G303R+P321H+E390A+N474L, H1*+G7A+D118T+S132D+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+G303R+N394D+R414M+G475K, H1*+G7A+T40G+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+M260D+N279S+K280Y+S302T+E390A+G475K, H1*+G7A+D118T+W140Y+R180*+S181*+T182S+E189P+N194F+L205Y+Y242F+E259G+M260W+N279S+K280Y+S302Y+E390A+G476A, H1*+G7A+D118T+W140Y+R180*+S181*+T182S+E189P+N194F+L205Y+Y242F+E259G+M260W+N279S+K280Y+G303R+E390A+G476A, H1*+G7A+D118T+W140Y+R180*+S181*+T182S+E189P+N194F+L205Y+Y242F+E259G+M260W+N279S+K280Y+G303R+E390A+G476A, H1*+G7A+D118T+W140Y+R180*+S181*+T182S+E189P+N194F+L205Y+Y242F+E259G+M260W+N279S+K280Y+S302Y+E390A+G475K, H1*+G7A+D118T+W140Y+R180*+S181*+T182S+E189P+N194F+L205Y+T224Y+Y242F+E259G+N279S+K280Y+G303R+E390A+N474L, H1*+G7A+D118T+W140Y+R180*+S181*+T182S+E189P+N194F+L205Y+Y242F+E259G+N279S+K280Y+S302Y+E390A+H420A+G475K, H1*+G7A+D118T+W140Y+P146H+R180*+S181*+T182S+E189P+N194F+L205Y+Y242F+E259G+N279S+K280Y+S302T+E390A+G475K, H1*+N3G+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+A185Q+E189P+N194F+L205Y+Y242F+E259G+N279S+K280Y+G303R+E390A+G475K, H1*+G7A+D118T+W140Y+R180*+S181*+T182S+E189P+N194F+L205Y+Y242F+E259G+F266Y+N279S+K280Y+G303R+E390A+G475K, H1*+G7A+D118T+W140Y+R180*+S181*+T182S+A185D+E189P+N194F+Y242F+E259G+F266Y+N279S+K280Y+G303R+E390A+G475K, H1*+G7A+D118T+W140Y+R180*+S181*+T182S+A185D+E189P+Y242F+S243Q+E259G+F266Y+N279S+K280Y+G303R+E390A+G475K, H1*+G7A+D118T+W140Y+R180Q+S181*+T182*+A185Q+E189P+Y242F+S243Q+E259G+F266Y+N279S+K280Y+G303R+E390A+G475K, H1*+G7A+D118T+W140Y+R180*+S181*+T182S+A185Q+E189P+L205Y+Y242F+E259G+F266Y+N279S+K280Y+G303R+E390A+G475K, H1*+G7A+D118T+W140Y+R180*+S181*+T182S+A185D+E189P+L205Y+Y242F+E259G+F266Y+N279S+K280Y+G303R+E390A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+T40G+D118T+W140Y+R180*+S181*+T182S+E189P+N194F+L205Y+Y242F+E259G+N279S+K280Y+G303R+E390A+G476A, H1*+G7A+T40G+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+G303R+N394D+N422V+G475K, H1*+G7A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+F261P+N279S+K280Y+G303R+E390A+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+T256D+N279S+K280Y+G303R+E390A+N470M+G475K, H1*+N3D+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+S302T+Y362V+E390A+G475K, H1*+G7A+D118T+W140Y+R180*+S181*+T182S+E189P+N194F+L205Y+Y242F+E259G+N279S+K280Y+G303R+Y362V+E390A+G476A, H1*+G7A+D118T+W140Y+R180*+S181*+T182S+E189P+N194F+L205Y+Y242F+E259G+M260D+N279S+K280Y+G303R+E390A+N474L, H1*+G7A+D118T+W140Y+R180*+S181*+T182S+E189P+N194F+L205Y+T224Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+Y362V+E390A+G475K, H1*+G7A+D118T+W140Y+R180*+S181*+T182S+E189P+N194F+L205Y+Y242F+E259G+M260W+N279S+K280Y+S302T+E390A+G475K, H1*+G7A+D118T+W140Y+R180*+S181*+T182S+E189P+N194F+L205Y+T224Y+Y242F+E259G+N279S+K280Y+G303R+E390A+G476A, H1*+G7A+D118T+W140Y+R180*+S181*+T182S+E189P+N194F+L205Y+Y242F+E259G+M260D+N279S+K280Y+G303R+E390A+N474F, H1*+G7A+D118T+W140Y+R180*+S181*+T182S+E189P+N194F+L205Y+Y242F+E259G+N279S+K280Y+G303R+E390A+H420P+G475K, H1*+G7A+D118T+W140Y+R180*+S181*+T182S+E189P+N194F+L205Y+Y242F+E259G+N279S+K280Y+G303R+E390A+N470M+G476A, H1*+G7A+D118T+W140Y+R180*+S181*+T182S+E189P+N194F+L205Y+Y242F+E259G+N279S+K280Y+S302T+E390A+H420A+G475K, H1*+G7A+D118T+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+N279S+K280Y+G303R+N394W+H420P+G475K, H1*+G7A+D118T+W140Y+R180*+S181*+T182S+E189P+N194F+L205Y+Y242F+E259G+N279S+K280Y+S302T+H323W+E390A+G475K, H1*+G7A+R37A+D118T+W140Y+R180*+S181*+T182S+E189P+N194F+L205Y+Y242F+E259G+N279S+K280Y+S302Y+E390A+N474L, H1*+G7A+R37A+D118T+W140Y+R180*+S181*+T182S+E189P+N194F+L205Y+Y242F+E259G+N279S+K280Y+S302Y+E390A+G475K, H1*+G7A+R37A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302T+N394W+H420A+G475K, H1*+G7A+R37A+D118T+W140Y+R180*+S181*+T182S+E189P+N194F+L205Y+Y242F+E259G+N279S+K280Y+S302Y+N394D+G475K, H1*+G7A+R37N+D118T+W140Y+R180*+S181*+T182S+E189P+N194F+L205Y+Y242F+E259G+N279S+K280Y+S302T+N394D+G475K, H1*+G7A+R37A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302Y+E390A+N474L, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302Y+E390A+G475K, H1*+G7A+R37A+D118T+W140Y+R180*+S181*+T182S+E189P+N194F+L205Y+T224F+Y242F+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+H420A+G475K, H1*+G7A+R37N+D118T+W140Y+R180*+S181*+T182S+E189P+N194F+L205Y+T224Y+Y242F+N279S+K280Y+G303R+E390A+G475K, H1*+G7A+D118T+W140Y+R180*+S181*+T182S+E189P+N194F+L205Y+Y242F+E259G+N279S+K280Y+G303R+N394W+H420A+G475K, H1*+G7A+D118T+W140Y+R180*+S181*+T182S+E189P+N194F+L205Y+Y242F+E259G+N279S+K280Y+G303R+N394W+H420A+N474L, H1*+G7A+D118T+W140Y+P146H+R180*+S181*+T182S+E189P+N194F+L205Y+Y242F+E259G+N279S+K280Y+S302T+E390A+G476A, H1*+G7A+D118T+W140Y+P146H+R180*+S181*+T182S+E189P+N194F+L205Y+Y242F+E259G+N279S+K280Y+S302Y+E390A+N474L, H1*+G7A+D118T+W140Y+R180*+S181*+T182S+A185Q+E189P+N194F+Y242F+E259G+F266Y+N279S+K280Y+G303R+E390A+G475K, H1*+G7A+D118T+W140Y+R180Q+S181*+T182*+A185Q+E189P+N194F+Y242F+E259G+F266Y+N279S+K280Y+G303R+E390A+G475K, H1*+G7A+D118T+W140Y+R180*+S181*+T182S+A185Q+E189P+Y242F+S243Q+E259G+F266Y+N279S+K280Y+G303R+E390A+G475K, H1*+N3D+G7A+D118T+W140Y+R180*+S181*+T182S+E189P+N194F+L205Y+Y242F+E259G+N279S+K280Y+G303R+E390A+G475K, H1*+G7A+D118T+W140Y+R180*+S181*+T182S+E189P+N194F+L205Y+Y242F+E259G+N279S+K280Y+G303R+E390A+N470M+G475K, H1*+G7A+D118T+W140Y+R180*+S181*+T182S+E189P+N194F+L205Y+Y242F+E259G+N279S+K280Y+S302Y+Y371N+E390A+G475K, H1*+G7A+D118T+Y135E+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+T224F+Y242F+E259G+N279S+K280Y+S302T+E390A+G475K, H1*+G7A+D118T+W140Y+R180*+S181*+T182S+E189P+N194F+L205Y+Y242F+E259G+M260D+N279S+K280Y+G303R+N394W+G475K, H1*+G7A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+Y242F+T256D+N279S+K280Y+G303R+E390A+G475K, H1*+G7A+R37A+D118T+W140Y+R180*+S181*+T182S+E189P+N194F+L205Y+T224Y+Y242F+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+E259G+N279S+K280Y+S302T+E390A+N470M+G475K, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+S132L+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+H420A+G476A, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302Y+E390A+G475K, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302T+E390A+G475K, H1*+G7A+D118T+S132D+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+S302Y+N394D+G475K H1*+G7A+D118T+S132L+W140Y+R180*+S181*+T182S+E189P+N194F+L205Y+Y242F+E259G+N279S+K280Y+S302Y+N394W+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+H420A+G476H, H1*+G7A+D118T+W140Y+R180*+S181*+T182S+E189P+N194F+L205Y+T224F+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+S302Y+E390A+G475K, H1*+G7A+D118T+Q138V+W140Y+R180*+S181*+T182S+E189P+N194F+L205Y+Y242F+E259G+N279S+K280Y+S302Y+E390A+G475K, H1*+G7A+D118T+S132L+W140Y+R180*+S181*+T182S+E189P+N194F+L205Y+Y242F+E259G+N279S+K280Y+S302Y+N394W+G475K, H1*+G7A+Q98L+D118T+W140Y+R180*+S181*+T182S+E189P+N194F+L205Y+Y242F+E259G+N279S+K280Y+G303R+E390A+G475K, H1*+G7A+R37A+D118T+W140Y+R180*+S181*+T182S+E189P+N194F+L205Y+Y242F+E259G+N279S+K280Y+G303R+E390A+G475K, H1*+N3D+G7A+D118T+W140Y+R180*+S181*+T182D+E189P+N194F+Y242F+E259G+N279S+K280Y+S302T+E390A+H420A+G475K, H1*+G7A+D118T+W140Y+R180*+S181*+T182S+A185Q+E189P+N194F+L205Y+T224Y+Y242F+N279S+K280Y+G303R+E390A+G476A, H1*+G7A+D118T+W140Y+R180*+S181*+T182S+A185Q+E189P+N194F+Y242F+E259G+M260D+N279S+K280Y+S302T+E390A+G475K, H1*+G7A+R37N+D118T+W140Y+R180*+S181*+T182S+A185Q+E189P+N194F+Y242F+E259G+N279S+K280Y+S302Y+E390A+G475K, H1*+G7A+D118T+W140Y+R180*+S181*+T182S+E189P+N194F+Y242F+E259G+M260D+F266Y+N279S+K280Y+S302T+E390A+G475K, H1*+G7A+D118T+W140Y+R180*+S181*+T182S+E189P+N194F+Y242F+E259G+M260W+F266Y+N279S+K280Y+G303R+E390A+G476A, H1*+G7A+D118T+W140Y+R180*+S181*+T182D+E189P+N194F+Y242F+E259G+M260D+F266Y+N279S+K280Y+S302T+E390A+G475K, H1*+G7A+R37N+D118T+W140Y+R180*+S181*+T182D+E189P+N194F+Y242F+E259G+F266Y+N279S+K280Y+S302Y+E390A+G475K, H1*+G7A+D118T+W140Y+R180*+S181*+T182S+A185Q+E189P+N194F+T224Y+Y242F+F266Y+N279S+K280Y+G303R+E390A+G476A, H1*+G7A+D118T+W140Y+R180*+S181*+T182S+A185Q+E189P+N194F+Y242F+E259G+F266Y+N279S+K280Y+S302T+E390A+G475K, H1*+G7A+D118T+W140Y+R180*+S181*+T182S+A185Q+E189P+N194F+Y242F+E259G+F266Y+N279S+K280Y+G303R+E390A+G476A, H1*+G7A+D118T+W140Y+R180*+S181*+T182S+E189P+N194F+Y242F+E259G+M260D+N279S+K280Y+S302Y+E390A+N470M+G475K, H1*+G7A+D118T+W140Y+R180*+S181*+T182D+E189P+N194F+Y242F+E259G+M260D+N279S+K280Y+S302Y+E390A+N470M+G475K, H1*+G7A+D118T+W140Y+R180*+S181*+T182S+A185Q+E189P+N194F+Y242F+E259G+M260W+N279S+K280Y+G303R+E390A+G476A, H1*+G7A+D118T+W140Y+R180*+S181*+T182D+E189P+N194F+Y242F+E259G+M260D+N279S+K280Y+S302Y+E390A+N470M+G475K, H1*+G7A+D118T+W140Y+R180*+S181*+T182D+A185Q+E189P+N194F+Y242F+E259G+F266Y+N279S+K280Y+G303R+E390A+G475K, H1*+G7A+D118T+W140Y+R180*+S181*+T182S+A185Q+E189P+N196I+Y242F+E259G+F266Y+N279S+K280Y+G303R+E390A+G475K, H1*+G7A+D118T+W140Y+R180*+S181*+T182S+A185D+E189P+Y242F+E259G+F266Y+N279S+K280Y+G303R+E390A+H420Y+G475K, H1*+G7A+D118T+W140Y+R180*+S181*+A185Q+E189P+N194F+L205Y+Y242F+E259G+F266Y+N279S+K280Y+G303R+E390A+G475K, H1*+G7A+D118T+W140Y+R180*+S181*+T182S+E189P+N194F+Y242F+E259G+M260D+N279S+K280Y+S302Y+E390A+N470M+G475K, H1*+G7A+D118T+W140Y+R180*+S181*+T182S+A185Q+E189P+N194F+Y242F+E259G+M260W+N279S+K280Y+G303R+E390A+G476A and

H1*+G7A+D118T+W140Y+R180*+S181*+T182D+E189P+N194F+L205Y+T224Y+Y242F+F266Y+N279S+K280Y+G303R+E390A+G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding to H1*+N3D+G7A+D118T+W140Y+R180*+S181*+T182S+E189P+N194F+L205Y+Y242F+E259G+N279S+K280Y+S302T+V365I+E390A+N474L, H1*+N3D+G7A+D118T+W140Y+R180*+S181*+T182S+E189P+N194F+L205Y+Y242F+E259G+N279S+K280Y+S302T+E390A+H420A+G475K, H1*+N3D+G7A+D118T+W140Y+R180*+S181*+T182S+E189P+N194F+L205Y+Y242F+E259G+N279S+K280Y+S302T+E390A+H420A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+R37A+D118T+W140Y+R180*+S181*+T182S+E189P+N194F+L205Y+Y242F+E259G+N279S+K280Y+G303R+N394D+N474F, H1*+N3D+G7A+D118T+W140Y+R180*+S181*+T182S+E189P+N194F+L205Y+Y242F+E259G+N279S+K280Y+S302Y+E390A+H420A+G475K, H1*+N3D+G7A+D118T+W140Y+R180*+S181*+T182S+E189P+N194F+L205Y+Y242F+E259G+N279S+K280Y+G303R+E390D+H420P+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+D118T+W140Y+R180*+S181*+T182S+E189P+N194F+L205Y+Y242F+E259G+N279S+K280Y+G303R+N394W+H420A+G475K, H1*+G7A+R37N+D118T+W140Y+R180*+S181*+T182S+E189P+N194F+L205Y+Y242F+E259G+N279S+K280Y+G303R+E390A+H420A+G475K, H1*+N3D+G7A+D118T+W140Y+R180*+S181*+T182S+E189P+N194F+L205Y+Y242F+E259G+N279S+K280Y+G303R+E390A+H420A+G475K, H1*+N3D+G7A+D118T+W140Y+R180*+S181*+T182S+E189P+N194F+L205Y+Y242F+E259G+N279S+K280Y+G303R+E390A+H420A+N474L, H1*+G7A+R37N+D118T+W140Y+R180*+S181*+T182S+E189P+N194F+L205Y+Y242F+E259G+N279S+K280Y+G303R+N394W+H420A+G475K, H1*+N3D+G7A+D118T+W140Y+R180*+S181*+T182S+E189P+N194F+L205Y+Y242F+E259G+N279S+K280Y+G303R+E390A+H420P+G475K, H1*+G7A+D118T+Q138V+W140Y+R180*+S181*+T182S+E189P+N194F+L205Y+Y242F+E259G+N279S+K280Y+G303R+H323W+E390A+N474L, H1*+G7A+D118T+W140Y+R180*+S181*+T182S+E189P+N194F+L205Y+T224F+Y242F+E259G+N279S+K280Y+G303R+N394W+H420A+G475K, H1*+G7A+D118T+W140Y+R180*+S181*+T182S+E189P+N194F+L205Y+Y242F+E259G+N279S+K280Y+G303R+P321H+Q360Y+E390A+G476A, H1*+G7A+D118T+W140Y+R180*+S181*+T182S+E189P+N194F+L205Y+Y242F+E259G+M260D+N279S+K280Y+S302Y+E390A+N470M+G475K, H1*+N3D+G7A+D118T+W140Y+R180*+S181*+T182S+E189P+N194F+L205Y+Y242F+E259G+N279S+K280Y+S302T+E390A+H420A+G475K, H1*+G7A+D118T+S132L+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+A185Q+E189P+N194F+L205Y+Y242F+E259G+F266Y+N279S+K280Y+G303R+E390A+G475K, H1*+G7A+D118T+W140Y+R180*+S181*+T182S+A185Q+E189P+N194F+L205N+Y242F+E259G+F266Y+N279S+K280Y+G303R+E390A+G475K, H1*+G7A+D118T+W140Y+R180Q+S181*+T182*+A185Q+E189P+N194F+L205Y+Y242F+E259G+F266Y+N279S+K280Y+G303R+E390A+G475K, H1*+N3D+G7A+D118T+W140Y+R180*+S181*+T182S+E189P+N194F+L205Y+Y242F+E259G+N279S+K280Y+S302T+E390A+H420A+G475K, H1*+G7A+Q98L+D118T+W140Y+R180*+S181*+T182S+E189P+N194F+L205Y+Y242F+E259G+N279S+K280Y+G303R+N394D+H420A+G475K, H1*+G7A+D118T+W140Y+R180*+S181*+T182S+E189P+N194F+L205Y+Y242F+E259G+M260D+N279S+K280Y+G303R+Y362V+E390A+G475K, H1*+G7A+D118T+Y135E+W140Y+R180*+S181*+T182S+E189P+N194F+L205Y+T224F+Y242F+E259G+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+Y371N+E390A+G475K, H1*+G7A+D118T+Y135E+W140Y+R180*+S181*+T182S+E189P+N194F+L205Y+Y242F+E259G+N279S+K280Y+G303R+P321Y+E390A+G475K, H1*+G7A+D118T+S132D+W140Y+R180*+S181*+T182S+E189P+N194F+L205Y+Y242F+E259G+N279S+K280Y+G303R+P321Y+E390A+G475K, H1*+G7A+D118T+S132D+W140Y+R180*+S181*+T182S+E189P+N194F+L205Y+Y242F+E259G+N279S+K280Y+G303R+Y362V+E390A+N474L, H1*+G7A+D118T+W140Y+R180*+S181*+T182S+E189P+N194F+L205Y+Y242F+E259G+M260D+N279S+K280Y+G303R+E390A+H420A+G475K, H1*+G7A+D118T+Y135E+W140Y+R180*+S181*+T182S+E189P+N194F+L205Y+Y242F+E259G+N279S+K280Y+G303R+N394W+H420A+G475K, H1*+N3D+G7A+D118T+W140Y+R180*+S181*+T182S+E189P+N194F+L205Y+Y242F+E259G+N279S+K280Y+G303R+N394D+H420A+G475K, H1*+G7A+D118T+Q138V+W140Y+R180*+S181*+T182S+E189P+N194F+L205Y+Y242F+E259G+N279S+K280Y+G303R+E390A+H420Q+G475K, H1*+G7A+R37N+D118T+W140Y+R180*+S181*+T182S+E189P+N194F+L205Y+Y242F+E259G+N279S+K280Y+G303R+E390A+H420P+G476H, H1*+G7A+R37A+Q98L+D118T+W140Y+R180*+S181*+T182S+E189P+N194F+L205Y+Y242F+E259G+N279S+K280Y+S302Y+E390A+G475K, H1*+G7A+R37A+D118T+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+M260D+N279S+K280Y+G303R+E390A+H420P+G476A, H1*+G7A+R37A+D118T+W140Y+R180*+S181*+T182S+E189P+N194F+L205Y+Y242F+E259G+N279S+K280Y+S302Y+E390A+H420A+G475K, H1*+G7A+D118T+Y135E+W140Y+R180*+S181*+T182S+E189P+N194F+L205Y+Y242F+E259G+N279S+K280Y+S302T+Y371N+E390A+G475K, H1*+G7A+D118T+W140Y+R180*+S181*+T182S+E189P+N194F+L205Y+Y242F+E259G+N279S+K280Y+G303R+H323W+E390A+H420A+N474L, H1*+G7A+D118T+Y135E+W140Y+R180*+S181*+T182S+E189P+N194F+L205Y+Y242F+E259G+N279S+K280Y+G303R+E390A+R414M+G476H, H1*+G7A+R37N+D118T+W140Y+R180*+S181*+T182S+E189P+N194F+L205Y+Y242F+E259G+N279S+K280Y+S302Y+E390A+H420A+G475K, H1*+G7A+R37A+D118T+W140Y+R180*+S181*+T182S+E189P+N194F+L205Y+Y242F+E259G+N279S+K280Y+S302T+E390A+H420A+G476A, H1*+G7A+R37N+D118T+W140Y+R180*+S181*+T182S+E189P+N194F+L205Y+Y242F+E259G+N279S+K280Y+S302Y+E390A+H420A+G475K, H1*+G7A+R37A+D118T+W140Y+R180*+S181*+T182S+E189P+N194F+L205Y+Y242F+E259G+N279S+K280Y+S302Y+E390A+H420A+G476H, H1*+G7A+R37A+D118T+W140Y+R180*+S181*+T182S+E189P+N194F+L205Y+Y242F+E259G+N279S+K280Y+S302Y+N394D+H420A+G475K, H1*+G7A+R37N+D118T+W140Y+R180*+S181*+T182S+E189P+N194F+L205Y+Y242F+E259G+N279S+K280Y+S302T+E390A+H420A+G475K, H1*+G7A+R37A+D118T+W140Y+R180*+S181*+T182S+E189P+N194F+L205Y+Y242F+E259G+N279S+K280Y+S302Y+E390A+H420A+G475K, H1*+G7A+R37N+D118T+W140Y+R180*+S181*+T182S+E189P+N194F+L205Y+Y242F+E259G+N279S+K280Y+S302T+E390A+H420A+G475K, H1*+G7A+R37A+D118T+W140Y+R180*+S181*+T182S+E189P+N194F+L205Y+Y242F+E259G+N279S+K280Y+S302T+E390A+H420A+G475K, H1*+G7A+R37N+D118T+W140Y+R180*+S181*+T182S+E189P+N194F+L205Y+Y242F+E259G+N279S+K280Y+S302Y+E390A+H420A+G476H, H1*+G7A+R37N+D118T+W140Y+R180*+S181*+T182S+E189P+N194F+L205Y+Y242F+E259G+N279S+K280Y+S302T+N394D+H420A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+R37A+D118T+W140Y+R180*+S181*+T182S+E189P+N194F+L205Y+Y242F+E259G+N279S+K280Y+G303R+E390A+G475K, H1*+G7A+R37A+D118T+W140Y+R180*+S181*+T182S+E189P+N194F+L205Y+Y242F+E259G+M260D+N279S+K280Y+G303R+N394W+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+Y242F+E259G+M260D+N279S+K280Y+G303R+N394D+G475K, H1*+G7A+R37N+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+A185Q+E189P+N194F+L205Q+Y242F+E259G+F266Y+N279S+K280Y+G303R+E390A+G475K, H1*+N3G+G7A+V104M+D118T+W140Y+R180*+S181*+T182S+E189P+N194F+L205Y+Y242F+E259G+N279S+K280Y+S302Y+N394W+G475K, H1*+N3G+G7A+D118T+W140Y+R180*+S181*+T182S+E189P+N194F+L205Y+Y242F+E259G+N279S+K280Y+G303R+N394W+H420A+G475K, H1*+N3D+G7A+D118T+W140Y+R180*+S181*+T182S+E189P+N194F+L205Y+Y242F+E259G+N279S+K280Y+G303R+E390A+H420A+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+E390A+N470M+G475K, H1*+G7A+R37A+D118T+W140Y+R180*+S181*+T182S+E189P+N194F+L205Y+Y242F+E259G+M260D+N279S+K280Y+G303R+N394W+G475K, H1*+G7A+R37A+D118T+W140Y+R180*+S181*+T182S+E189P+N194F+L205Y+Y242F+E259G+N279S+K280Y+G303R+E390A+H420A+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+R37A+D118T+W140Y+R180*+S181*+T182S+E189P+N194F+L205Y+T224Y+Y242F+E259G+N279S+K280Y+G303R+N394W+G475K, H1*+G7A+R37A+D118T+W140Y+R180*+S181*+T182S+E189P+N194F+L205Y+Y242F+E259G+F261P+N279S+K280Y+G303R+E390A+G475K, H1*+G7A+R37A+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+T224Y+Y242F+E259G+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+H420A+G475K, H1*+N3D+G7A+D118T+W140Y+R180*+S181*+T182S+E189P+N194F+L205Y+Y242F+E259G+M260D+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3G+G7A+R37N+D118T+W140Y+R180*+S181*+T182S+E189P+N194F+L205Y+Y242F+E259G+N279S+K280Y+G303R+E390A+G475K, H1*+G7A+D118T+W140Y+R180*+S181*+T182S+E189P+N194F+L205Y+T224F+Y242F+E259G+N279S+K280Y+G303R+E390A+H420A+G475K, H1*+G7A+D118T+S132L+W140Y+R180*+S181*+T182S+E189P+N194F+L205Y+T224F+Y242F+E259G+N279S+K280Y+S302Y+E390A+G475K, H1*+G7A+D118T+Q138V+W140Y+R180*+S181*+T182S+E189P+N194F+L205Y+T224F+Y242F+E259G+N279S+K280Y+S302Y+E390A+G475K, H1*+G7A+R37N+D118T+W140Y+R180*+S181*+T182S+E189P+N194F+L205Y+Y242F+T256D+E259G+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+N394D+G475K, N3G+G7A+R37V+D118T+W140M+R180*+S181*+T182S+E189P+N194F+L205Y+Y242F+E259G+N279S+K280Y+G303R+E390A+H420A+G475K, H1*+G7A+R37N+D118T+W140Y+R180*+S181*+T182S+E189P+N194F+L205Y+Y242F+E259G+N279S+K280Y+G303R+H323W+N394D+G475K, H1*+N3D+G7A+D118T+W140Y+R180*+S181*+T182S+E189P+N194F+L205Y+Y242F+E259G+N279S+K280Y+G303R+H323W+E390A+G475K, H1*+N3D+G7A+D118T+W140Y+R180*+S181*+T182S+E189P+N194F+L205Y+Y242F+E259G+N279S+K280Y+G303R+H323W+E390A+G475K, H1*+G7A+D118T+S132L+W140Y+R180*+S181*+T182D+E189P+N194F+L205Y+Y242F+E259G+M260D+N279S+K280Y+G303R+E390A+G475K, H1*+G7A+D118T+W140Y+R180*+S181*+T182S+A185Q+E189P+N194F+Y242F+E259G+M260D+N279S+K280Y+S302Y+E390A+N470M+G475K, H1*+N3D+G7A+D118T+W140Y+R180*+S181*+T182S+A185Q+E189P+N194F+Y242F+E259G+N279S+K280Y+S302T+E390A+H420A+G475K, H1*+G7A+D118T+W140Y+R180*+S181*+T182S+A185Q+E189P+N194F+L205Y+Y242F+E259G+M260W+N279S+K280Y+G303R+E390A+G476A, H1*+G7A+D118T+W140Y+R180*+S181*+T182S+E189P+N194F+Y242F+E259G+M260D+F266Y+N279S+K280Y+S302Y+E390A+N470M+G475K, H1*+G7A+D118T+S132L+W140Y+R180*+S181*+T182S+E189P+N194F+Y242F+E259G+M260D+F266Y+N279S+K280Y+G303R+E390A+G475K, H1*+N3D+G7A+D118T+W140Y+R180*+S181*+T182D+E189P+N194F+Y242F+E259G+F266Y+N279S+K280Y+S302T+E390A+H420A+G475K, H1*+G7A+D118T+W140Y+R180*+S181*+T182S+A185Q+E189P+N194F+Y242F+E259G+M260W+F266Y+N279S+K280Y+G303R+E390A+G476A, H1*+G7A+R37N+D118T+W140Y+R180*+S181*+T182S+A185Q+E189P+N194F+L205Y+Y242F+E259G+N279S+K280Y+S302Y+E390A+G475K, H1*+G7A+D118T+W140Y+R180*+S181*+T182S+A185Q+E189P+N194F+L205Y+Y242F+E259G+M260D+N279S+K280Y+S302T+E390A+G475K and H1*+N3D+G7A+D118T+W140Y+R180*+S181*+T182D+E189P+N194F+L205Y+Y242F+E259G+N279S+K280Y+S302T+E390A+H420A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding to H1*+G7A+N106D+A109G+F116W+D123N+N126D+T131I+T165V+N174S+R180*+S181*+T182I+F202Y+Y242F+E259G+N279S+G303R+E390A+G475K, H1*+N3D+G7A+D118T+W140Y+R180*+S181*+T182S+E189P+N194F+L205Y+Y242F+E259G+N279S+K280Y+G303R+Y362V+E390A+H420A+N474L, H1*+N3D+G7A+K93R+D118T+W140Y+R180*+S181*+T182S+E189P+N194F+L205Y+Y242F+E259G+N279S+K280Y+G303R+E390A+H420A+N474L, H1*+N3D+G7A+R37N+D118T+Q138V+W140Y+R180*+S181*+T182S+E189P+N194F+L205Y+Y242F+E259G+N279S+K280Y+G303R+E390A+G475K, H1*+G7A+D118T+Y135E+W140Y+R180*+S181*+T182S+E189P+N194F+L205Y+Y242F+E259G+M260D+N279S+K280Y+G303R+W346N+E390A+G475K, H1*+G7A+D118T+Y135E+W140Y+R180*+S181*+T182S+E189P+N194F+L205Y+Y242F+E259G+F261P+N279S+K280Y+G303R+Y362V+E390A+G475K, H1*+N3G+G7A+D118T+Q138V+W140Y+R180*+S181*+T182S+E189P+N194F+L205Y+Y242F+E259G+N279S+K280Y+G303R+E390A+H420A+G476A, H1*+G7A+R37N+D118T+S132D+W140Y+R180*+S181*+T182S+E189P+N194F+L205Y+Y242F+E259G+N279S+K280Y+G303R+E390A+N470M+G475K, H1*+N3D+G7A+R37A+D118T+W140Y+R180*+S181*+T182S+E189P+N194F+L205Y+Y242F+E259G+N279S+K280Y+S302Y+N394D+H420A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3G+G7A+R37N+D118T+W140Y+R180*+S181*+T182S+E189P+N194F+L205Y+Y242F+E259G+N279S+K280Y+G303R+N394W+H420A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3D+G7A+R37N+D118T+W140Y+R180*+S181*+T182S+E189P+N194F+L205Y+Y242F+E259G+N279S+K280Y+G303R+G361M+H420A+G475K, H1*+N3D+G7A+R37A+D118T+W140Y+R180*+S181*+T182S+E189P+N194F+L205Y+Y242F+E259G+N279S+K280Y+G303R+E390A+H420A+G475K, H1*+N3D+G7A+D118T+S132D+W140Y+R180*+S181*+T182S+E189P+N194F+L205Y+Y242F+E259G+N279S+K280Y+G303R+E390A+H420A+G475K, H1*+G7A+R37A+D118T+W140Y+R180*+S181*+T182S+E189P+N194F+L205Y+Y242F+E259G+M260W+N279S+K280Y+G303R+E390A+N470M+G475K, H1*+G7A+R37N+D118T+W140Y+R180*+S181*+T182S+E189P+N194F+L205Y+Y242F+E259G+M260W+N279S+K280Y+G303R+E390A+H420A+G475K, H1*+G7A+R37N+D118T+W140Y+R180*+S181*+T182S+E189P+N194F+L205Y+Y242F+E259G+M260D+N279S+K280Y+G303R+N394W+H420A+G475K, H1*+G7A+R37A+D118T+W140Y+R180*+S181*+T182S+E189P+N194F+L205Y+Y242F+E259G+M260D+N279S+K280Y+G303R+E390A+N470M+G475K, H1*+G7A+R37A+D118T+W140Y+R180*+S181*+T182S+E189P+N194F+L205Y+Y242F+E259G+M260D+N279S+K280Y+G303R+N394D+H420A+G475K, H1*+G7A+R37N+D118T+W140Y+N174K+R180*+S181*+T182S+E189P+N194F+L205Y+Y242F+E259G+M260D+N279S+K280Y+G303R+N394W+G475K, H1*+G7A+R37A+D118T+W140Y+R180*+S181*+T182S+E189P+N194F+L205Y+Y242F+E259G+N279S+K280Y+G303R+P321Y+L354V+N394D+G475K, H1*+G7A+R37N+D118T+W140Y+R180*+S181*+T182S+E189P+N194F+L205Y+Y242F+E259G+N279S+K280Y+G303R+H323W+Q360M+E390A+G475K, H1*+N3D+G7A+D118T+W140Y+R180*+S181*+T182S+A185Q+E189P+N194F+Y242F+E259G+F266Y+N279S+K280Y+S302T+E390A+H420A+G475K and

H1*+G7A+A31S+R37N+D118T+W140Y+R180*+S181*+T182S+A185Q+E189P+N194F+Y242F+E259G+F266Y+N279S+K280Y+S302Y+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding to

H1*+G7A+D118T+S132L+W140Y+R180*+S181*+T182S+A185Q+E189P+N194F+L205Y+Y242F+E259G+M260D+F266Y+N279S+K280Y+G303R+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding to

H1*+N54S+V56T+K72R+G113Q+F116Q+D118Q+D123N+N126D+T131I+K142G+D144N+W167F+K172G+N174S+S181T+T182*+G183*+F202Y+E390A+F395Y+K457R+S472R+V473C+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 2 comprising modifications in the positions corresponding to

H1*+N54S+V56T+K72R+N106D+A109G+G113Q+F116Q+D118Q+D123N+N126D+T131I+K142G+D144N+W167F+K172G+N174S+S181T+T182*+G183*+F202Y+E390A+F395Y+K457R+S472R+V473C+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to E169I+R180*+S181*, T114K+R180*+S181*, W167A+R180*+S181*, E169V+R180*+S181*, R180*+S181*+W188C, W167D+R180*+S181*, Y177D+R180*+S181*, W167S+R180*+S181*, W167E+R180*+S181*, W167P+R180*+S181*, R180*+S181*+L205R, R180*+S181*+A203F, W159L+R180*+S181*, Y160P+R180*+S181*, W159H+R180*+S181*, K178A+R180*+S181*, R180*+S181*+G183P, Q136T+R180*+S181*, K142P+R180*+S181*, K142E+R180*+S181*, Y152L+R180*+S181*, A111L+R180*+S181*, G110K+R180*+S181*, A111G+R180*+S181* and V117G+R180*+S181*, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to R180*+S181*+E193+H323N, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to G7H+Q98L+G110K+R180*+S181*, G7H+W167P+R180*+S181*+G455R and Q98L+V103A+G110K+R180*+S181*, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to A60V+Q98L+G110K+R180*+S181*+A383D, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to F116L+D123N+N126D+T131I+R180*+S181*+F202Y and Q98L+G110K+S132D+Y135E+R180*+S181*+T315A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to H1*+G7A+R180*+S181*+Y242F+E259G+N279S+G303R+E390A+G475K, H1*+G7A+S181*+T182*+Y242F+E259G+N279S+G303R+E390A+G475K and H1*+G7S+R180*+S181*+Y242F+E259G+N279S+G303R+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to H1*+G7A+R180*+S181*+T182S+Y242F+E259G+N279S+G303R+E390A+G475K, H1*+G7A+R180*+T182L+G183*+Y242F+E259G+N279S+G303R+E390A+G475K, H1*+G7A+S181 L+T182*+G183*+Y242F+E259G+N279S+G303R+E390A+G475K, H1*+G7A+R180S+T182*+G183*+Y242F+E259G+N279S+G303R+E390A+G475K, H1*+G7A+A60P+R180*+S181*+Y242F+E259G+N279S+G303R+E390A+G475K, H1*+G7A+M105L+R180*+S181*+Y242F+E259G+N279S+G303R+E390A+G475K, H1*+G7A+M105I+R180*+S181*+Y242F+E259G+N279S+G303R+E390A+G475K, H1*+G7A+R180*+S181*+T182D+Y242F+E259G+N279S+G303R+E390A+G475K, H1*+G7A+R180*+S181*+T182E+Y242F+E259G+N279S+G303R+E390A+G475K, H1*+G7A+R180*+S181*+T182G+Y242F+E259G+N279S+G303R+E390A+G475K, H1*+G7A+R180*+S181*+T182M+Y242F+E259G+N279S+G303R+E390A+G475K, H1*+G7A+R180*+S181*+A185K+Y242F+E259G+N279S+G303R+E390A+G475K, H1*+G7A+R180*+S181*+A185D+Y242F+E259G+N279S+G303R+E390A+G475K, H1*+G7A+R180*+S181*+A185Q+Y242F+E259G+N279S+G303R+E390A+G475K, H1*+G7A+R180*+S181*+A185N+Y242F+E259G+N279S+G303R+E390A+G475K, H1*+G7A+R180*+S181*+A185R+Y242F+E259G+N279S+G303R+E390A+G475K, H1*+G7A+R180*+S181*+A185E+Y242F+E259G+N279S+G303R+E390A+G475K, H1*+G7A+R180*+S181*+E189P+Y242F+E259G+N279S+G303R+E390A+G475K, H1*+G7A+R180*+S181*+L205Y+Y242F+E259G+N279S+G303R+E390A+G475K, H1*+G7A+R180*+S181*+L205F+Y242F+E259G+N279S+G303R+E390A+G475K, H1*+G7A+R180*+S181*+Y242F+S243Q+E259G+N279S+G303R+E390A+G475K, H1*+G7A+R180*+S181*+Y242F+F244L+E259G+N279S+G303R+E390A+G475K, H1*+G7A+R180*+S181*+Y242F+F244I+E259G+N279S+G303R+E390A+G475K, H1*+G7A+R180*+S181*+Y242F+E259G+F266Y+N279S+G303R+E390A+G475K, H1*+G7A+R180*+S181*+N194F+Y242F+E259G+N279S+G303R+E390A+G475K, H1*+G7A+R180*+S181*+N196F+Y242F+E259G+N279S+G303R+E390A+G475K, H1*+G7A+R180*+S181*+Y242F+E259G+N279S+G303R+M322G+E390A+G475K, H1*+G7A+T40N+R180*+S181*+Y242F+E259G+N279S+G303R+E390A+G475K, H1*+G7S+Q98L+R180*+S181*+Y242F+E259G+N279S+G303R+E390A+G475K, H1*+G7S+R180*+S181*+Y242F+E259G+F261K+N279S+G303R+E390A+G475K, H1*+G7S+R180*+S181*+Y242F+E259G+N279S+G303R+Y362V+E390A+G475K, H1*+G7A+R180*+S181*+Y242F+E259G+N279S+G303R+Y362V+E390A+G475K, H1*+G7S+R180*+S181*+Y242F+E259G+F261S+N279S+G303R+E390A+G475K, H1*+G7A+R180*+S181*+Y242F+E259G+N279S+S286L+G303R+E390A+G475K, H1*+G7A+G110K+R180*+S181*+Y242F+E259G+N279S+G303R+E390A+G475K, H1*+G7A+W140S+R180*+S181*+Y242F+E259G+N279S+G303R+E390A+G475K, H1*+G7A+W167P+R180*+S181*+Y242F+E259G+N279S+G303R+E390A+G475K, H1*+G7A+R180*+S181*+Y242F+E259G+N279S+G303R+H323A+E390A+G475K, H1*+G7A+R180*+S181*+Y242F+E259G+N279S+G303R+H320T+E390A+G475K, H1*+G7A+R180*+S181*+Y242F+E259G+N279S+G303R+P321H+E390A+G475K, H1*+G7A+R180*+S181*+Y242F+E259G+N279S+G303R+M322Y+E390A+G475K, H1*+G7A+R180*+S181*+Y242F+E259G+N279S+G303R+Q360Y+E390A+G475K, H1*+G7A+Q98L+R180*+S181*+Y242F+E259G+N279S+G303R+E390A+G475K, H1*+G7A+R180*+S181*+Y242F+E259G+F261S+N279S+G303R+E390A+G475K, H1*+G7H+G110K+R180*+S181*+Y242F+E259G+N279S+G303R+E390A+G475K, H1*+G7A+Q98L+R180*+S181*+Y242F+E259G+N279S+G303R+E390A+G475K, H1*+G7A+R180*+S181*+Y242F+E259G+F261K+N279S+G303R+E390A+G475K and L63F+W140Y+L173F+R180*+S181*+N194F+L205Y+Y242F+E259G+N279S+H323W, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to H1*+G7A+R180*+S181*+N194F+L205Y+Y242F+E259G+N279S+G303R+E390A+G475K, H1*+G7A+R180*+S181*+N194F+L205F+Y242F+E259G+N279S+G303R+E390A+G475K, H1*+G7A+R180*+S181*+N196F+L205Y+Y242F+E259G+N279S+G303R+E390A+G475K, H1*+G7A+R180*+S181*+E189P+Y242F+E259G+N276F+N279S+G303R+E390A+G475K, H1*+G7A+R180*+S181*+E189P+Y242F+E259G+N276Y+N279S+G303R+E390A+G475K, H1*+G7A+R180*+S181*+E189P+Y242F+E259G+N276W+N279S+G303R+E390A+G475K, H1*+G7A+R180*+S181*+E189P+Y242F+E259G+N279S+K280Y+G303R+E390A+G475K, H1*+G7A+R180*+S181*+E189P+Y242F+E259G+N279S+K280W+G303R+E390A+G475K, H1*+G7A+R180*+S181*+Y242F+E259G+N279S+G303R+M322G+Q360W+E390A+G475K, H1*+G7A+R180*+S181*+Y242F+E259G+N279S+G303R+E359W+Q360W+E390A+G475K, H1*+G7S+R180*+S181*+Y242F+E259G+F261S+N279S+G303R+Y362V+E390A+G475K, H1*+G7A+T40N+Q98L+R180*+S181*+Y242F+E259G+N279S+G303R+E390A+G475K, H1*+G7S+T40N+Q98L+R180*+S181*+Y242F+E259G+N279S+G303R+E390A+G475K, H1*+G7A+D123N+I176V+R180*+S181*+Y242F+E259G+N279S+G303R+E390A+G475K, H1*+G7A+R180*+S181*+Y242F+E259G+N279S+G303R+M322G+Q360W+E390A+G475K, H1*+G7A+T40N+Q98L+R180*+S181*+Y242F+E259G+N279S+G303R+E390A+G475K, H1*+G7A+R180*+S181*+Y242F+E259G+N279S+S286K+G303R+H323A+E390A+G475K, H1*+G7A+R180*+S181*+Y242F+E259G+N279S+G303R+H320T+H323A+E390A+G475K, H1*+G7A+R180*+S181*+Y242F+E259G+N279S+G303R+P321H+M322Y+E390A+G475K, H1*+G7A+R180*+S181*+Y242F+E259G+N279S+G303R+P321H+Q360Y+E390A+G475K, H1*+G7A+R180*+S181*+Y242F+E259G+N279S+G303R+M322Y+Q360Y+E390A+G475K, H1*+G7S+T40N+R180*+S181*+Y242F+E259G+N279S+G303R+Y362V+E390A+G475K, H1*+G7S+R180*+S181*+Y242F+E259G+F261S+N279S+G303R+Y362V+E390A+G475K, H1*+G7S+T40N+Q98L+R180*+S181*+Y242F+E259G+N279S+G303R+E390A+G475K, H1*+G7A+R180*+S181*+Y242F+E259G+F261S+N279S+G303R+Y362V+E390A+G475K, H1*+G7A+R180*+S181*+Y242F+E259G+N279S+S286L+G303R+H320T+E390A+G475K, H1*+G7H+Q98L+G110K+R180*+S181*+Y242F+E259G+N279S+G303R+E390A+G475K, H1*+G7A+Q98L+G110K+R180*+S181*+Y242F+E259G+N279S+G303R+E390A+G475K, H1*+G7H+Q98L+G110K+R180*+S181*+Y242F+E259G+N279S+G303R+E390A+G475K, H1*+G7A+Q98L+G110K+R180*+S181*+Y242F+E259G+N279S+G303R+E390A+G475K, H1*+G7A+R180*+S181*+Y242F+E259G+N279S+S286L+G303R+Y362V+E390A+G475K, H1*+G7A+R180*+S181*+Y242F+E259G+N279S+W283N+S286K+G303R+E390A+G475K, H1*+G7A+R180*+S181*+Y242F+E259G+N279S+W283N+G303R+H320T+E390A+G475K, H1*+G7A+R180*+S181*+Y242F+E259G+N279S+W283N+G303R+H323A+E390A+G475K, H1*+G7A+R180*+S181*+Y242F+E259G+N279S+S286K+G303R+H320T+E390A+G475K, H1*+G7A+R180*+S181*+Y242F+E259G+N279S+S286K+G303R+Y362V+E390A+G475K, H1*+G7A+R180*+S181*+Y242F+E259G+N279S+G303R+M322G+E359W+E390A+G475K and W140Y+L173F+R180*+S181*+N194F+L205Y+N225S+Y242F+E259G+N279S+G303R+H323A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to H1*+G7A+R180*+S181*+E189P+L205F+Y242F+E259G+N276F+N279S+G303R+E390A+G475K, H1*+G7A+R180*+S181*+E189P+L205F+Y242F+E259G+N276Y+N279S+G303R+E390A+G475K, H1*+G7A+R180*+S181*+E189P+L205F+Y242F+E259G+N276W+N279S+G303R+E390A+G475K, H1*+G7A+R180*+S181*+E189P+L205F+Y242F+E259G+N279S+K280F+G303R+E390A+G475K, H1*+G7A+R180*+S181*+E189P+L205F+Y242F+E259G+N279S+K280Y+G303R+E390A+G475K, H1*+G7A+R180*+S181*+E189P+L205F+Y242F+E259G+N279S+K280W+G303R+E390A+G475K, H1*+G7A+R180*+S181*+E189P+L205Y+Y242F+E259G+N276F+N279S+G303R+E390A+G475K, H1*+G7A+R180*+S181*+E189P+L205Y+Y242F+E259G+N276Y+N279S+G303R+E390A+G475K, H1*+G7A+R180*+S181*+E189P+L205Y+Y242F+E259G+N276W+N279S+G303R+E390A+G475K, H1*+G7A+R180*+S181*+E189P+L205Y+Y242F+E259G+N279S+K280F+G303R+E390A+G475K, H1*+G7A+R180*+S181*+E189P+L205Y+Y242F+E259G+N279S+K280Y+G303R+E390A+G475K, H1*+G7A+R180*+S181*+E189P+L205Y+Y242F+E259G+N279S+K280W+G303R+E390A+G475K, H1*+G7A+G113S+F116W+D123N+S181*+T182*+Y242F+E259G+N279S+G303R+E390A+G475K, H1*+G7S+T40N+Q98L+R180*+S181*+Y242F+E259G+N279S+G303R+Y362V+E390A+G475K, H1*+G7A+R180*+S181*+Y242F+E259G+N279S+G303R+M322G+E359W+Q360W+E390A+G475K, H1*+G7A+R180*+S181*+Y242F+E259G+F261S+N279S+S286K+G303R+Y362V+E390A+G475K, H1*+G7A+R180*+S181*+Y242F+E259G+N279S+G303R+P321H+M322Y+Q360Y+E390A+G475K, H1*+G7A+G113S+F116W+D123N+R180*+S181*+Y242F+E259G+N279S+G303R+E390A+G475K, H1*+G7S+T40N+Q98L+R180*+S181*+Y242F+E259G+N279S+G303R+H320T+E390A+G475K, H1*+G7S+T40N+Q98L+R180*+S181*+Y242F+E259G+N279S+G303R+Y362V+E390A+G475K, H1*+G7A+1137F+R180*+S181*+Y242F+E259G+F261S+N279S+G303R+Y362V+E390A+G475K, H1*+G7A+R180*+S181*+Y242F+E259G+F261S+N279S+S286K+G303R+Y362V+E390A+G475K, H1*+G7A+R180*+S181*+Y242F+E259G+N279S+W283N+G303R+H320T+H323A+E390A+G475K, H1*+G7A+Q98L+R180*+S181*+Y242F+E259G+F261K+N279S+G303R+Y362V+E390A+G475K, H1*+G7H+Q98L+R180*+S181*+Y242F+E259G+N279S+G303R+A383D+E390A+D405G+G475K, H1*+G7A+Q98L+G110K+R180*+S181*+Y242F+T256A+E259G+N279S+G303R+E390A+G475K, H1*+G7A+Q98L+G110K+R180*+S181*+Y242F+E259G+N279S+G303R+A383D+E390A+G475K, H1*+G7A+W140Y+R180*+S181*+N194F+L205Y+Y242F+E259G+N279S+G303R+E390A+G476A, H1*+G7A+W140Y+R180*+S181*+N194F+L205Y+Y242F+E259G+N279S+G303R+N394W+G475K, H1*+G7A+W140Y+R180*+S181*+N194F+L205Y+Y242F+E259G+N279S+G303R+E390A+N474L, H1*+G7A+W140Y+R180*+S181*+N194F+L205Y+Y242F+E259G+N279S+S302T+E390A+G475K, H1*+G7A+W140Y+R180*+S181*+N194F+L205Y+Y242F+E259G+N279S+G303R+E390A+G475E, H1*+G7A+W140Y+R180*+S181*+N194F+L205Y+Y242F+E259G+N279S+G303R+E390A+N474F, H1*+G7A+W140Y+R180*+S181*+N194F+L205Y+Y242F+E259G+N279S+S302Y+E390A+G475K, H1*+G7A+W140Y+R180*+S181*+N194F+L205Y+Y242F+T256D+N279S+G303R+E390A+G475K, H1*+G7A+R37A+W140Y+R180*+S181*+N194F+L205Y+Y242F+E259G+N279S+G303R+G475K, H1*+W140Y+R180*+S181*+N194F+L205Y+Y242F+E259G+N279S+G303R+Q360Y+E390A+G475K, H1*+G7A+R37N+W140Y+R180*+S181*+N194F+L205Y+Y242F+E259G+N279S+G303R+G475K, H1*+W140Y+R180*+S181*+N194F+L205Y+Y242F+E259G+N279S+G303R+E390A+R414M+G475K, H1*+W140Y+R180*+S181*+N194F+L205Y+Y242F+E259G+F261P+N279S+G303R+E390A+G475K, H1*+W140Y+R180*+S181*+N194F+L205Y+Y242F+E259G+N279S+G303R+H323W+E390A+G475K, H1*+W140Y+R180*+S181*+N194F+L205Y+Y242F+E259G+N279S+G303R+P321Y+E390A+G475K, H1*+W140Y+R180*+S181*+N194F+L205Y+Y242F+E259G+N279S+G303R+Q360M+E390A+G475K, H1*+G7A+W140Y+R180*+S181*+Y242F+S243Q+E259G+N279S+K280Y+G303R+E390A+G475K, H1*+G7A+W140Y+R180*+S181*+Y242F+E259G+F266Y+N279S+K280Y+G303R+E390A+G475K and H1*+G7A+W140Y+R180*+S181*+T182D+A185Q+Y242F+E259G+N279S+G303R+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding: H1*+G7A+R180*+S181*+Y242F+E259G+N279S+W283N+S286K+G303R+H320T+H323A+E390A+G475K, H1*+G7A+W140Y+R180*+S181*+T182D+N194F+L205Y+Y242F+E259G+N279S+G303R+E390A+G475K, H1*+G7A+W140Y+R180*+S181*+T182D+N194F+L205V+Y242F+E259G+N279S+G303R+E390A+G475K, H1*+G7A+W140Y+R180*+S181*+T182S+N194F+L205Y+Y242F+E259G+N279S+G303R+E390A+G475K, H1*+G7A+W140Y+R180*+S181*+T182S+N194F+L205V+Y242F+E259G+N279S+G303R+E390A+G475K, H1*+G7A+D123N+W140Y+R180*+S181*+N194F+L205V+Y242F+E259G+N279S+G303R+E390A+G475K, H1*+G7A+D123N+W140Y+R180*+S181*+N194F+L205Y+Y242F+E259G+N279S+G303R+E390A+G475K, H1*+G7A+Q98L+G110K+P146S+R180*+S181*+Y242F+E259G+N279S+G303R+A383D+E390A+G475K, H1*+N3S+G7A+Q98L+G110K+R180*+S181*+Y242F+V252+E259G+N279S+G303R+E390A+G475K, H1*+G7A+W140Y+R180*+S181*+N194F+L205V+Y242F+E259G+F266Y+N279S+G303R+E390A+G475K, H1*+G7A+W140Y+R180*+S181*+A185Q+N194F+L205V+Y242F+E259G+N279S+G303R+E390A+G475K, H1*+G7A+W140Y+R180*+S181*+A185Q+N194F+L205Y+Y242F+E259G+N279S+G303R+E390A+G475K, H1*+G7A+W140Y+R180*+S181*+E189P+N194F+Y242F+E259G+N279S+K280Y+G303R+E390A+G475K, H1*+G7A+W140Y+R180*+S181*+E189P+N194F+Y242F+E259G+N279S+K280Y+G303R+E390A+G475K, H1*+G7A+W140Y+R180*+S181*+N194F+L205Y+Y242F+E259G+F266Y+N279S+G303R+E390A+G475K, H1*+G7A+W140Y+R180*+S181*+N194F+L205Y+Y242F+E259G+N279S+S302T+G303R+E390A+G475K, H1*+G7A+W140Y+R180*+S181*+N194F+L205Y+Y242F+E259G+N279S+G303R+E390A+N474F+G475K, H1*+G7A+W140Y+R180*+S181*+N194F+L205Y+Y242F+E259G+N279S+G303R+E390A+N422V+G475K, H1*+G7A+W140Y+R180*+S181*+N194F+L205Y+Y242F+E259G+F261P+N279S+G303R+E390A+G475K, H1*+G7A+W140Y+R180*+S181*+N194F+L205Y+Y242F+E259G+N279S+G303R+E390A+N470A+G475K, H1*+G7A+W140Y+R180*+S181*+N194F+L205Y+Y242L+E259G+N279S+G303R+W346N+E390A+G475K, H1*+G7A+W140Y+R180*+S181*+N194F+L205Y+Y242F+E259G+N279S+G303R+E390A+H420P+G475K, H1*+G7A+R26Y+W140Y+R180*+S181*+N194F+L205Y+Y242F+E259G+N279S+G303R+E390A+G475K, H1*+G7A+W140Y+R180*+S181*+N194F+L205Y+Y242F+E259G+N279S+G303R+Y371S+E390A+G475K, H1*+G7A+W140Y+R180*+S181*+T182D+A185Q+Y242F+E259G+N279S+K280Y+G303R+E390A+G475K, H1*+G7A+W140Y+R180*+S181*+A185Q+E189P+Y242F+E259G+N279S+K280Y+G303R+E390A+G475K, H1*+G7A+W140Y+R180*+S181*+E189P+Y242F+S243Q+E259G+N279S+K280Y+G303R+E390A+G475K, H1*+G7A+W140Y+R180*+S181*+T182D+Y242F+S243Q+E259G+N279S+K280Y+G303R+E390A+G475K, H1*+G7A+W140Y+R180*+S181*+Y242F+S243Q+E259G+F266Y+N279S+K280Y+G303R+E390A+G475K, H1*+G7A+A41D+W140Y+R180*+S181*+N194F+L205Y+Y242F+E259G+N279S+G303R+E390A+G475K and H1*+G7A+T40G+W140Y+R180*+S181*+N194F+L205Y+Y242F+E259G+N279S+G303R+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding: H1*+G7A+F116L+D123N+N126D+T131 I+S181*+T182*+F202Y+Y242F+E259G+N279S+G303R+E390A+G475K, H1*+G7A+F116L+D123N+N126D+T131 I+R180*+S181*+F202Y+Y242F+E259G+N279S+G303R+E390A+G475K, H1*+G7A+D118T+W140Y+R180*+S181*+T182D+N194F+L205Y+Y242F+E259G+N279S+G303R+E390A+G475K, H1*+G7A+F116N+W140Y+R180*+S181*+T182D+N194F+L205Y+Y242F+E259G+N279S+G303R+E390A+G475K, H1*+G7A+W140Y+R180*+S181*+E189P+N194F+L205Y+Y242F+E259G+N279S+K280Y+G303R+E390A+G475K, H1*+G7A+W140Y+R180*+S181*+T182D+E189P+N194F+L205Y+Y242F+E259G+N279S+G303R+E390A+G475K, H1*+G7A+T134D+W140Y+R180*+S181*+E189P+N194F+L205V+Y242F+E259G+N279S+G303R+E390A+G475K, H1*+G7A+D118T+W140Y+R180*+S181*+T182S+N194F+L205Y+Y242F+E259G+N279S+G303R+E390A+G475K, H1*+G7A+F116N+W140Y+R180*+S181*+T182S+N194F+L205Y+Y242F+E259G+N279S+G303R+E390A+G475K, H1*+G7A+W140Y+R180*+S181*+T182S+E189P+N194F+L205Y+Y242F+E259G+N279S+G303R+E390A+G475K, H1*+G7A+W140Y+R180*+S181*+T182S+E189P+N194F+L205V+Y242F+E259G+N279S+G303R+E390A+G475K, H1*+G7A+D123N+W140Y+I176V+R180*+S181*+N194F+L205V+Y242F+E259G+N279S+G303R+E390A+G475K, H1*+G7A+D123N+W140Y+I176V+R180*+S181*+N194F+L205Y+Y242F+E259G+N279S+G303R+E390A+G475K, H1*+G7A+Q98L+G110K+S132D+Y135E+R180*+S181*+Y242F+E259G+N279S+G303R+T315A+E390A+G475K, H1*+A60V+Q98L+W140Y+R180*+S181*+N194F+L205Y+Y242F+E259G+N279S+G303R+A383D+E390A+G475K, H1*+G7A+W140Y+T151A+R180*+S181*+N194F+L205Y+Y242F+K258V+E259V+N279S+G303R+E390A+G475K, H1*+N3S+G7A+N94D+Q98L+G110K+R180*+S181*+Y242F+V252I+E259G+N279S+G303R+E390A+G475K, H1*+G7A+W140Y+T151A+R180*+S181*+N194F+L205Y+Y242F+K258V+E259V+N279S+G303R+E390A+G475K, H1*+G7A+F116N+W140Y+R180*+S181*+N194F+L205Y+Y242F+E259G+F266Y+N279S+G303R+E390A+G475K, H1*+G7A+F116N+W140Y+R180*+S181*+A185Q+N194F+L205Y+Y242F+E259G+N279S+G303R+E390A+G475K, H1*+G7A+D118T+W140Y+R180*+S181*+E189P+N194F+Y242F+E259G+N279S+K280Y+G303R+E390A+G475K, H1*+G7A+D118T+W140Y+R180*+S181*+N194F+L205Y+Y242F+E259G+F266Y+N279S+G303R+E390A+G475K, H1*+G7A+D118T+W140Y+R180*+S181*+A185Q+N194F+L205Y+Y242F+E259G+N279S+G303R+E390A+G475K, H1*+G7A+W140Y+R180*+S181*+E189P+N194F+L205V+Y242F+E259G+N279S+K280Y+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+T182S+E189P+N194F+Y242F+E259G+N279S+K280Y+G303R+E390A+G475K, H1*+G7A+W140Y+R180*+S181*+T182D+N194F+L205V+Y242F+E259G+F266Y+N279S+G303R+E390A+G475K, H1*+G7A+W140Y+R180*+S181*+A185Q+N194F+L205V+Y242F+E259G+F266Y+N279S+G303R+E390A+G475K, H1*+G7A+W140Y+R180*+S181*+T182D+A185Q+N194F+L205V+Y242F+E259G+N279S+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+T182S+E189P+N194F+Y242F+E259G+N279S+K280Y+G303R+E390A+G475K, H1*+G7A+W140Y+R180*+S181*+T182D+N194F+L205Y+Y242F+E259G+F266Y+N279S+G303R+E390A+G475K, H1*+G7A+W140Y+R180*+S181*+A185Q+N194F+L205Y+Y242F+E259G+F266Y+N279S+G303R+E390A+G475K, H1*+G7A+W140Y+R180*+S181*+T182D+A185Q+N194F+L205Y+Y242F+E259G+N279S+G303R+E390A+G475K, H1*+G7A+F116N+W140Y+R180*+S181*+E189P+N194F+Y242F+E259G+N279S+K280Y+G303R+E390A+G475K, H1*+G7A+W140Y+R180*+S181*+T182D+Y242F+S243Q+E259G+F266Y+N279S+K280Y+G303R+E390A+G475K, H1*+G7A+W140Y+R180*+S181*+T182D+E189P+Y242F+S243Q+E259G+N279S+K280Y+G303R+E390A+G475K, H1*+G7A+W140Y+R180*+S181*+A185Q+Y242F+E259G+F266Y+N279S+K280Y+G303R+E390A+T427L+G475K, H1*+G7A+W140Y+R180*+S181*+A185Q+Y242F+S243Q+E259G+F266Y+N279S+K280Y+G303R+E390A+G475K, H1*+G7A+W140Y+R180*+S181*+T182D+A185Q+Y242F+S243Q+E259G+F266Y+N279S+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+A185Q+N194F+Y242F+S243Q+E259G+N279S+K280Y+G303R+E390A+G475K, H1*+G7A+W140Y+R180*+S181*+T182D+N194F+Y242F+S243Q+E259G+N279S+K280Y+G303R+E390A+G475K, H1*+G7A+W140Y+R180*+S181*+A185Q+E189P+N194F+Y242F+E259G+N279S+K280Y+G303R+E390A+G475K, H1*+G7A+W140Y+R180*+S181*+E189P+L205Y+Y242F+S243Q+E259G+N279S+K280Y+G303R+E390A+G475K, H1*+G7A+W140Y+R180*+S181*+T182D+A185Q+N194F+Y242F+S243Q+E259G+N279S+G303R+E390A+G475K, H1*+G7A+W140Y+R180*+S181*+T182D+N194F+Y242F+S243Q+E259G+F266Y+N279S+G303R+E390A+G475K, H1*+G7A+W140Y+R180*+S181*+E189P+N194F+Y242F+E259G+F266Y+N279S+K280Y+G303R+E390A+G475K, H1*+G7A+W140Y+R180*+S181*+T182D+A185Q+N194F+Y242F+E259G+F266Y+N279S+G303R+E390A+G475K, H1*+G7A+W140Y+R180*+S181*+A185Q+N194F+Y242F+E259G+F266Y+N279S+K280Y+G303R+E390A+G475K, H1*+G7A+W140Y+R180*+S181*+A185Q+E189P+L205Y+Y242F+E259G+N279S+K280Y+G303R+E390A+G475K, H1*+G7A+W140Y+R180*+S181*+E189P+N194F+Y242F+E259G+F266Y+N279S+K280Y+S302T+E390A+G475K, H1*+G7A+W140Y+R180*+S181*+E189P+N194F+Y242F+E259G+M260D+N279S+K280Y+S302T+E390A+G475K, H1*+G7A+W140Y+R180*+S181*+E189P+N194F+Y242F+E259G+F266Y+N279S+K280Y+G303R+E390A+G476A and H1*+G7A+W140Y+R180*+S181*+E189P+N194F+Y242F+E259G+M260W+N279S+K280Y+G303R+E390A+G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding: H1*+G7A+D118T+W140Y+R180*+S181*+E189P+N194F+L205Y+Y242F+E259G+N279S+K280Y+G303R+E390A+G475K, H1*+G7A+F116N+W140Y+R180*+S181*+E189P+N194F+L205Y+Y242F+E259G+N279S+K280Y+G303R+E390A+G475K, H1*+G7A+F116N+W140Y+R180*+S181*+T182D+E189P+N194F+L205Y+Y242F+E259G+N279S+G303R+E390A+G475K, H1*+G7A+G113S+F116W+D123N+W140Y+R180*+S181*+N194F+L205V+Y242F+E259G+N279S+G303R+E390A+G475K, H1*+G7A+N106D+A109G+W140Y+R180*+S181*+N194F+F202Y+L205V+Y242F+E259G+N279S+G303R+E390A+G475K, H1*+G7A+G113S+F116W+D123N+W140Y+R180*+S181*+N194F+L205Y+Y242F+E259G+N279S+G303R+E390A+G475K, H1*+G7A+F116N+W140Y+R180*+S181*+T182D+E189P+N194F+Y242F+E259G+N279S+K280Y+G303R+E390A+G475K, H1*+G7A+F116N+W140Y+R180*+S181*+T182S+E189P+N194F+Y242F+E259G+N279S+K280Y+G303R+E390A+G475K, H1*+G7A+F116N+W140Y+R180*+S181*+T182D+N194F+L205Y+Y242F+E259G+F266Y+N279S+G303R+E390A+G475K, H1*+G7A+F116N+W140Y+R180*+S181*+A185Q+N194F+L205Y+Y242F+E259G+F266Y+N279S+G303R+E390A+G475K, H1*+G7A+F116N+W140Y+R180*+S181*+T182D+A185Q+N194F+L205Y+Y242F+E259G+N279S+G303R+E390A+G475K, H1*+G7A+D118T+W140Y+R180*+S181*+T182D+N194F+L205Y+Y242F+E259G+F266Y+N279S+G303R+E390A+G475K, H1*+G7A+D118T+W140Y+R180*+S181*+A185Q+N194F+L205Y+Y242F+E259G+F266Y+N279S+G303R+E390A+G475K, H1*+G7A+D118T+W140Y+R180*+S181*+T182D+A185Q+N194F+L205Y+Y242F+E259G+N279S+G303R+E390A+G475K, H1*+G7A+W140Y+R180*+S181*+T182D+E189P+N194F+L205V+Y242F+E259G+N279S+K280Y+G303R+E390A+G475K, H1*+G7A+W140Y+R180*+S181*+T182D+A185Q+N194F+L205V+Y242F+E259G+F266Y+N279S+G303R+E390A+G475K, H1*+G7A+W140Y+R180*+S181*+T182D+E189P+N194F+L205Y+Y242F+E259G+N279S+K280Y+G303R+E390A+G475K, H1*+G7A+W140Y+R180*+S181*+T182S+E189P+N194F+L205Y+Y242F+E259G+N279S+K280Y+G303R+E390A+G475K, H1*+G7A+W140Y+R180*+S181*+T182D+A185Q+N194F+L205Y+Y242F+E259G+F266Y+N279S+G303R+E390A+G475K, H1*+G7A+D118T+W140Y+R180*+S181*+T182D+E189P+N194F+Y242F+E259G+N279S+K280Y+G303R+E390A+G475K, H1*+G7A+D118T+W140Y+R180*+S181*+T182S+E189P+N194F+Y242F+E259G+N279S+K280Y+G303R+E390A+G475K, H1*+G7A+W140Y+R180*+S181*+T182D+A185Q+N194F+Y242F+S243Q+E259G+N279S+K280Y+G303R+E390A+G475K, H1*+G7A+W140Y+R180*+S181*+A185Q+N194F+Y242F+S243Q+E259G+F266Y+N279S+K280Y+G303R+E390A+G475K, H1*+G7A+W140Y+R180*+S181*+T182D+E189P+L205Y+Y242F+E259G+F266Y+N279S+K280Y+G303R+E390A+G475K, H1*+G7A+W140Y+R180*+S181*+A185Q+E189P+N194F+Y242F+E259G+F266Y+N279S+K280Y+G303R+E390A+G475K, H1*+G7A+W140Y+R180*+S181*+T182D+E189P+N194F+Y242F+S243Q+E259G+N279S+K280Y+G303R+E390A+G475K, H1*+D118T+W140Y+R180*+S181*+T182S+E189P+N194F+L205Y+Y242F+E259G+N279S+K280Y+G303R+E390A+G475K, H1*+G7A+W140Y+R180*+S181*+T182S+E189P+N194F+L205Y+Y242F+E259G+N279S+K280Y+G303R+E390A+G475K, H1*+G7A+D118T+R180*+S181*+T182S+E189P+N194F+L205Y+Y242F+E259G+N279S+K280Y+G303R+E390A+G475K, H1*+G7A+D118T+W140Y+R180*+S181*+E189P+N194F+L205Y+Y242F+E259G+N279S+K280Y+G303R+E390A+G475K, H1*+G7A+D118T+W140Y+R180*+S181*+T182S+N194F+L205Y+Y242F+E259G+N279S+K280Y+G303R+E390A+G475K, H1*+G7A+D118T+W140Y+R180*+S181*+T182S+E189P+L205Y+Y242F+E259G+N279S+K280Y+G303R+E390A+G475K, H1*+G7A+D118T+W140Y+R180*+S181*+T182S+E189P+N194F+Y242F+E259G+N279S+K280Y+G303R+E390A+G475K, H1*+G7A+D118T+W140Y+R180*+S181*+T182S+E189P+N194F+L205Y+E259G+N279S+K280Y+G303R+E390A+G475K, H1*+G7A+D118T+W140Y+R180*+S181*+T182S+E189P+N194F+L205Y+Y242F+N279S+K280Y+G303R+E390A+G475K, H1*+G7A+D118T+W140Y+R180*+S181*+T182S+E189P+N194F+L205Y+Y242F+E259G+K280Y+G303R+E390A+G475K, H1*+G7A+D118T+W140Y+R180*+S181*+T182S+E189P+N194F+L205Y+Y242F+E259G+N279S+G303R+E390A+G475K, H1*+G7A+D118T+W140Y+R180*+S181*+T182S+E189P+N194F+L205Y+Y242F+E259G+N279S+K280Y+E390A+G475K, H1*+G7A+D118T+W140Y+R180*+S181*+T182S+E189P+N194F+L205Y+Y242F+E259G+N279S+K280Y+G303R+G475K, H1*+G7A+D118T+W140Y+R180*+S181*+T182S+E189P+N194F+L205Y+Y242F+E259G+N279S+K280Y+G303R+E390A, H1*+G7A+D118T+W140Y+R180*+S181*+T182S+E189P+N194F+Y242F+E259G+N279S+K280Y+S302T+E390A+G475K, H1*+G7A+D118T+W140Y+R180*+S181*+T182D+E189P+N194F+Y242F+E259G+N279S+K280Y+G303R+E390A+G476A, H1*+G7A+R37N+W140Y+R180*+S181*+E189P+N194F+Y242F+E259G+F266Y+N279S+K280Y+S302Y+E390A+G475K, H1*+G7A+D118T+W140Y+R180*+S181*+T182S+E189P+N194F+T224Y+Y242F+N279S+K280Y+G303R+E390A+G476A, H1*+G7A+D118T+W140Y+R180*+S181*+T182D+E189P+N194F+T224Y+Y242F+N279S+K280Y+G303R+E390A+G476A, H1*+G7A+D118T+W140Y+R180*+S181*+T182D+A185Q+Y242F+S243Q+E259G+F266Y+N279S+G303R+E390A+G475K, H1*+G7A+D118T+W140Y+R180*+S181*+T182D+N194F+Y242F+S243Q+E259G+F266Y+N279S+G303R+E390A+G475K, H1*+G7A+D118T+W140Y+R180*+S181*+T182D+E189P+N194F+T224Y+Y242F+N279S+K280Y+G303R+E390A+G476A, H1*+G7A+D118T+W140Y+R180*+S181*+T182S+E189P+N194F+T224Y+Y242F+N279S+K280Y+G303R+E390A+G476A, H1*+G7A+D118T+W140Y+R180*+S181*+T182D+A185Q+N194F+Y242F+E259G+N279S+K280Y+S302T+E390A+G475K, H1*+G7A+W140Y+R180*+S181*+E189P+N194F+Y242F+E259G+M260D+N279S+K280Y+S302Y+E390A+N470A+G475K, H1*+G7A+S132L+W140Y+R180*+S181*+E189P+N194F+Y242F+E259G+M260D+N279S+K280Y+G303R+E390A+G475K, and T40N+F116L+D123N+N126D+N128Y+T131I+F155W+R180*+S181*+T182D+E189P+F202Y+K457N+P458S+G475K+G476E, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to H1*+G7A+G113A+F116W+D123N+T131V+Q136K+E169Q+K172Q+R180*+S181*+Y242F+E259G+N279S+G303R+E390A+G475K, H1*+G7A+F116N+W140Y+R180*+S181*+T182S+E189P+N194F+L205Y+Y242F+E259G+N279S+K280Y+G303R+E390A+G475K, H1*+N3S+G7A+N94D+Q98L+W140Y+R180*+S181*+N194F+L205Y+Y242F+V252+E259G+N279S+G303R+E390A+G475K, H1*+G7A+F116N+W140Y+R180*+S181*+T182D+E189P+N194F+L205Y+Y242F+E259G+N279S+K280Y+G303R+E390A+G475K, H1*+G7A+F116N+W140Y+R180*+S181*+T182D+A185Q+N194F+L205Y+Y242F+E259G+F266Y+N279S+G303R+E390A+G475K, H1*+G7A+D118T+W140Y+R180*+S181*+T182S+E189P+N194F+L205Y+Y242F+E259G+N279S+K280Y+G303R+E390A+G475K, H1*+G7A+D118T+W140Y+R180*+S181*+T182D+A185Q+N194F+L205Y+Y242F+E259G+F266Y+N279S+G303R+E390A+G475K, H1*+G7A+D118T+W140Y+R180*+S181*+T182D+E189P+N194F+L205Y+Y242F+E259G+N279S+K280Y+G303R+E390A+G475K, H1*+G7A+W140Y+R180*+S181*+T182D+A185Q+E189P+N194F+Y242F+S243Q+E259G+N279S+K280Y+G303R+E390A+G475K, H1*+G7A+W140Y+R180*+S181*+A185Q+E189P+N194F+Y242F+S243Q+E259G+F266Y+N279S+K280Y+G303R+E390A+G475K, H1*+G7A+W140Y+R180*+S181*+T182D+A185Q+E189P+N194F+Y242F+E259G+F266Y+N279S+K280Y+G303R+E390A+G475K, H1*+G7A+W140Y+R180*+S181*+A185Q+E189P+N194F+L205Y+Y242F+S243Q+E259G+N279S+K280Y+G303R+E390A+G475K, H1*+G7A+W140Y+R180*+S181*+T182D+E189P+L205Y+Y242F+S243Q+E259G+N279S+K280Y+G303R+E390A+I409L+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+S302Y+N394W+G475K, H1*+G7A+D118T+W140Y+R180*+S181*+T182S+E189P+N194F+L205Y+Y242F+E259G+N279S+K280Y+S302T+N394W+G475K, H1*+G7A+D118T+W140Y+R180*+S181*+T182S+E189P+N194F+L205Y+Y242F+E259G+N279S+K280Y+S302T+E390A+G476A, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302Y+E390A+G475K H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+G303R+E390A+N474L, H1*+G7A+D118T+W140Y+R180*+S181*+T182S+E189P+N194F+L205Y+T224Y+Y242F+N279S+K280Y+G303R+E390A+G476A H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302Y+E390A+N474F, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+G475K, H1*+G7A+D118T+W140Y+R180*+S181*+T182S+E189P+N194F+L205V+Y242F+E259G+N279S+K280Y+G303R+E390A+G475K, H1*+G7A+D118T+W140Y+R180*+S181*+T182S+E189P+N194F+L205N+Y242F+E259G+N279S+K280Y+G303R+E390A+G475K, H1*+G7A+D118T+W140Y+R180*+S181*+T182S+E189P+N194F+L205Q+Y242F+E259G+N279S+K280Y+G303R+E390A+G475K, H1*+G7A+D118T+W140Y+R180*+S181*+A185Q+E189P+N194F+L205Y+Y242F+E259G+N279S+K280Y+G303R+E390A+G475K, H1*+G7A+D118T+W140Y+R180*+S181*+T182S+A185Q+E189P+Y242F+E259G+F266Y+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+N474F, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+N394D+G475K, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302T+N394W+G475K, H1*+G7A+D118T+W140Y+R180*+S181*+T182S+E189P+N194F+L205Y+Y242F+E259G+N279S+K280Y+S302T+N394D+G475K, H1*+G7A+D118T+W140Y+R180*+S181*+T182S+A185D+E189P+Y242F+E259G+F266Y+N279S+K280Y+G303R+E390A+G475K, H1*+G7A+D118T+W140Y+R180Q+S181*+T182*+A185Q+E189P+Y242F+E259G+F266Y+N279S+K280Y+G303R+E390A+G475K, H1*+G7A+D118T+W140Y+R180*+S181*+T182S+E189P+N194F+Y242F+E259G+M260D+N279S+K280Y+S302T+E390A+G475K, H1*+G7A+R37N+D118T+W140Y+R180*+S181*+T182S+E189P+N194F+Y242F+E259G+N279S+K280Y+S302Y+E390A+G475K, H1*+G7A+D118T+W140Y+R180*+S181*+T182S+E189P+N194F+Y242F+E259G+M260W+N279S+K280Y+G303R+E390A+G476A, H1*+G7A+D118T+W140Y+R180*+S181*+T182D+E189P+N194F+Y242F+E259G+M260D+N279S+K280Y+S302T+E390A+G475K, H1*+G7A+R37N+D118T+W140Y+R180*+S181*+T182D+E189P+N194F+Y242F+E259G+N279S+K280Y+S302Y+E390A+G475K, H1*+G7A+D118T+W140Y+R180*+S181*+T182S+A185Q+E189P+N194F+Y242F+E259G+N279S+K280Y+G303R+E390A+G476A, H1*+G7A+D118T+W140Y+R180*+S181*+T182S+E189P+N194F+T224Y+Y242F+F266Y+N279S+K280Y+G303R+E390A+G476A, H1*+G7A+D118T+W140Y+R180*+S181*+T182S+E189P+N194F+Y242F+E259G+F266Y+N279S+K280Y+S302T+E390A+G475K, H1*+G7A+D118T+W140Y+R180*+S181*+T182S+E189P+N194F+Y242F+E259G+F266Y+N279S+K280Y+G303R+E390A+G476A, H1*+G7A+D118T+W140Y+R180*+S181*+T182D+E189P+N194F+Y242F+E259G+F266Y+N279S+K280Y+S302T+E390A+G475K, H1*+N3D+G7A+W140Y+R180*+S181*+E189P+N194F+Y242F+E259G+F266Y+N279S+K280Y+S302T+E390A+H420A+G475K, H1*+G7A+D118T+W140Y+R180*+S181*+T182D+E189P+N194F+Y242F+E259G+M260W+N279S+K280Y+G303R+E390A+G476A, H1*+G7A+D118T+W140Y+R180*+S181*+T182S+A185Q+E189P+N194F+T224Y+Y242F+N279S+K280Y+G303R+E390A+G476A, H1*+G7A+D118T+W140Y+R180*+S181*+T182S+A185Q+E189P+N194F+Y242F+E259G+N279S+K280Y+S302T+E390A+G475K, H1*+G7A+D118T+W140Y+R180*+S181*+T182D+E189P+N194F+Y242F+E259G+F266Y+N279S+K280Y+G303R+E390A+G476A, H1*+G7A+D118T+W140Y+R180*+S181*+T182D+A185Q+E189P+N194F+T224Y+Y242F+N279S+K280Y+G303R+E390A+G476A and H1*+G7A+D118T+W140Y+R180*+S181*+T182S+A185Q+E189P+N194F+Y242F+E259G+N279S+K280Y+S302T+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to H1*+G7A+F116L+D123N+N126D+T131I+W140Y+R180*+S181*+N194F+F202Y+L205V+Y242F+E259G+N279S+G303R+E390A+G475K, H1*+G7A+F116L+D123N+N126D+T131I+W140Y+R180*+S181*+N194F+F202Y+L205Y+Y242F+E259G+N279S+G303R+E390A+G475K, H1*+G7A+W140Y+R180*+S181*+T182D+A185Q+E189P+N194F+L205Y+Y242F+S243Q+E259G+N279S+K280Y+G303R+E390A+G475K, H1*+G7A+W140Y+R180*+S181*+A185Q+E189P+N194F+L205Y+Y242F+S243Q+E259G+F266Y+N279S+K280Y+G303R+E390A+G475K, H1*+G7A+W140Y+R180*+S181*+T182D+A185Q+E189P+N194F+L205Y+Y242F+E259G+F266Y+N279S+K280Y+G303R+E390A+G475K, H1*+N3D+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+H420A+G475K, H1*+G7A+D118T+W140Y+R180*+S181*+T182S+E189P+N194F+L205Y+Y242F+E259G+N279S+K280Y+S302Y+E390A+H420A+G475K, H1*+N3D+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+H420A+G475K, H1*+G7A+D118T+W140Y+R180*+S181*+T182S+E189P+N194F+L205Y+Y242F+E259G+N279S+K280Y+G303R+E390A+H420A+G476A, H1*+N3D+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+H420A+N474L, H1*+G7A+D118T+W140Y+R180*+S181*+T182S+E189P+N194F+L205Y+Y242F+E259G+N279S+K280Y+G303R+N394W+H420P+N474L, H1*+G7A+G73L+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302Y+E390A+G475K, H1*+G7A+R37N+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302T+E390A+G476H, H1*+G7A+D118T+W140Y+R180*+S181*+T182S+E189P+N194F+L205Y+Y242F+E259G+N279S+K280Y+G303R+N394D+H420A+G475K, H1*+G7A+D118T+W140Y+R180*+S181*+T182S+E189P+N194F+L205Y+Y242F+E259G+N279S+K280Y+G303R+E390A+H420P+G475K, N3G+G7A+D118T+W140Y+R180*+S181*+T182S+E189P+N194F+L205Y+Y242F+E259G+N279S+K280Y+G303R+E390A+H420A+N474L, H1*+G7A+D118T+Y135E+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+G303R+N394W+H420A+G475K, H1*+G7A+V103I+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+M260D+N279S+K280Y+S302Y+E390A+G475K, H1*+G7A+D118T+W140Y+P146H+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+F261P+N279S+K280Y+G303R+E390A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+Q98L+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+G303R+P321H+E390A+G475K, H1*+G7A+D118T+W140Y+R180*+S181*+T182S+E189P+N194F+L205Y+Y242F+E259G+N279S+K280Y+G303R+P321H+E390A+N474L, H1*+G7A+D118T+S132D+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+G303R+N394D+R414M+G475K, H1*+G7A+T40G+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+M260D+N279S+K280Y+S302T+E390A+G475K, H1*+G7A+D118T+W140Y+R180*+S181*+T182S+E189P+N194F+L205Y+Y242F+E259G+M260W+N279S+K280Y+S302Y+E390A+G476A, H1*+G7A+D118T+W140Y+R180*+S181*+T182S+E189P+N194F+L205Y+Y242F+E259G+M260W+N279S+K280Y+G303R+E390A+G476A, H1*+G7A+D118T+W140Y+R180*+S181*+T182S+E189P+N194F+L205Y+Y242F+E259G+M260W+N279S+K280Y+G303R+E390A+G476A, H1*+G7A+D118T+W140Y+R180*+S181*+T182S+E189P+N194F+L205Y+Y242F+E259G+M260W+N279S+K280Y+S302Y+E390A+G475K, H1*+G7A+D118T+W140Y+R180*+S181*+T182S+E189P+N194F+L205Y+T224Y+Y242F+E259G+N279S+K280Y+G303R+E390A+N474L, H1*+G7A+D118T+W140Y+R180*+S181*+T182S+E189P+N194F+L205Y+Y242F+E259G+N279S+K280Y+S302Y+E390A+H420A+G475K, H1*+G7A+D118T+W140Y+P146H+R180*+S181*+T182S+E189P+N194F+L205Y+Y242F+E259G+N279S+K280Y+S302T+E390A+G475K, H1*+N3G+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+A185Q+E189P+N194F+L205Y+Y242F+E259G+N279S+K280Y+G303R+E390A+G475K, H1*+G7A+D118T+W140Y+R180*+S181*+T182S+E189P+N194F+L205Y+Y242F+E259G+F266Y+N279S+K280Y+G303R+E390A+G475K, H1*+G7A+D118T+W140Y+R180*+S181*+T182S+A185D+E189P+N194F+Y242F+E259G+F266Y+N279S+K280Y+G303R+E390A+G475K, H1*+G7A+D118T+W140Y+R180*+S181*+T182S+A185D+E189P+Y242F+S243Q+E259G+F266Y+N279S+K280Y+G303R+E390A+G475K, H1*+G7A+D118T+W140Y+R180Q+S181*+T182*+A185Q+E189P+Y242F+S243Q+E259G+F266Y+N279S+K280Y+G303R+E390A+G475K, H1*+G7A+D118T+W140Y+R180*+S181*+T182S+A185Q+E189P+L205Y+Y242F+E259G+F266Y+N279S+K280Y+G303R+E390A+G475K, H1*+G7A+D118T+W140Y+R180*+S181*+T182S+A185D+E189P+L205Y+Y242F+E259G+F266Y+N279S+K280Y+G303R+E390A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+T40G+D118T+W140Y+R180*+S181*+T182S+E189P+N194F+L205Y+Y242F+E259G+N279S+K280Y+G303R+E390A+G476A, H1*+G7A+T40G+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+G303R+N394D+N422V+G475K, H1*+G7A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+F261P+N279S+K280Y+G303R+E390A+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+T256D+N279S+K280Y+G303R+E390A+N470M+G475K, H1*+N3D+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+S302T+Y362V+E390A+G475K, H1*+G7A+D118T+W140Y+R180*+S181*+T182S+E189P+N194F+L205Y+Y242F+E259G+N279S+K280Y+G303R+Y362V+E390A+G476A, H1*+G7A+D118T+W140Y+R180*+S181*+T182S+E189P+N194F+L205Y+Y242F+E259G+M260D+N279S+K280Y+G303R+E390A+N474L, H1*+G7A+D118T+W140Y+R180*+S181*+T182S+E189P+N194F+L205Y+T224Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+Y362V+E390A+G475K, H1*+G7A+D118T+W140Y+R180*+S181*+T182S+E189P+N194F+L205Y+Y242F+E259G+M260W+N279S+K280Y+S302T+E390A+G475K, H1*+G7A+D118T+W140Y+R180*+S181*+T182S+E189P+N194F+L205Y+T224Y+Y242F+E259G+N279S+K280Y+G303R+E390A+G476A, H1*+G7A+D118T+W140Y+R180*+S181*+T182S+E189P+N194F+L205Y+Y242F+E259G+M260D+N279S+K280Y+G303R+E390A+N474F, H1*+G7A+D118T+W140Y+R180*+S181*+T182S+E189P+N194F+L205Y+Y242F+E259G+N279S+K280Y+G303R+E390A+H420P+G475K, H1*+G7A+D118T+W140Y+R180*+S181*+T182S+E189P+N194F+L205Y+Y242F+E259G+N279S+K280Y+G303R+E390A+N470M+G476A, H1*+G7A+D118T+W140Y+R180*+S181*+T182S+E189P+N194F+L205Y+Y242F+E259G+N279S+K280Y+S302T+E390A+H420A+G475K, H1*+G7A+D118T+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+N279S+K280Y+G303R+N394W+H420P+G475K, H1*+G7A+D118T+W140Y+R180*+S181*+T182S+E189P+N194F+L205Y+Y242F+E259G+N279S+K280Y+S302T+H323W+E390A+G475K, H1*+G7A+R37A+D118T+W140Y+R180*+S181*+T182S+E189P+N194F+L205Y+Y242F+E259G+N279S+K280Y+S302Y+E390A+N474L, H1*+G7A+R37A+D118T+W140Y+R180*+S181*+T182S+E189P+N194F+L205Y+Y242F+E259G+N279S+K280Y+S302Y+E390A+G475K, H1*+G7A+R37A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302T+N394W+H420A+G475K, H1*+G7A+R37A+D118T+W140Y+R180*+S181*+T182S+E189P+N194F+L205Y+Y242F+E259G+N279S+K280Y+S302Y+N394D+G475K, H1*+G7A+R37N+D118T+W140Y+R180*+S181*+T182S+E189P+N194F+L205Y+Y242F+E259G+N279S+K280Y+S302T+N394D+G475K, H1*+N3D+G7A+D118T+W140Y+R180*+S181*+T182S+E189P+N194F+L205Y+Y242F+E259 G+N279S+K280Y+S302T+E390A+G475K, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302Y+E390A+N474L, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302Y+E390A+G475K, H1*+G7A+R37A+D118T+W140Y+R180*+S181*+T182S+E189P+N194F+L205Y+T224F+Y242F+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+H420A+G475K, H1*+G7A+R37N+D118T+W140Y+R180*+S181*+T182S+E189P+N194F+L205Y+T224Y+Y242F+N279S+K280Y+G303R+E390A+G475K, H1*+G7A+D118T+W140Y+R180*+S181*+T182S+E189P+N194F+L205Y+Y242F+E259G+N279S+K280Y+G303R+N394W+H420A+G475K, H1*+G7A+D118T+W140Y+R180*+S181*+T182S+E189P+N194F+L205Y+Y242F+E259G+N279S+K280Y+G303R+N394W+H420A+N474L, H1*+G7A+D118T+W140Y+P146H+R180*+S181*+T182S+E189P+N194F+L205Y+Y242F+E259G+N279S+K280Y+S302T+E390A+G476A, H1*+G7A+D118T+W140Y+P146H+R180*+S181*+T182S+E189P+N194F+L205Y+Y242F+E259G+N279S+K280Y+S302Y+E390A+N474L, H1*+G7A+D118T+W140Y+R180*+S181*+T182S+A185Q+E189P+N194F+Y242F+E259G+F266Y+N279S+K280Y+G303R+E390A+G475K, H1*+G7A+D118T+W140Y+R180Q+S181*+T182*+A185Q+E189P+N194F+Y242F+E259G+F266Y+N279S+K280Y+G303R+E390A+G475K, H1*+G7A+D118T+W140Y+R180*+S181*+T182S+A185Q+E189P+Y242F+S243Q+E259G+F266Y+N279S+K280Y+G303R+E390A+G475K, H1*+N3D+G7A+D118T+W140Y+R180*+S181*+T182S+E189P+N194F+L205Y+Y242F+E259G+N279S+K280Y+G303R+E390A+G475K, H1*+G7A+D118T+W140Y+R180*+S181*+T182S+E189P+N194F+L205Y+Y242F+E259G+N279S+K280Y+G303R+E390A+N470M+G475K, H1*+G7A+D118T+W140Y+R180*+S181*+T182S+E189P+N194F+L205Y+Y242F+E259G+N279S+K280Y+S302Y+Y371N+E390A+G475K, H1*+G7A+D118T+Y135E+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+T224F+Y242F+E259G+N279S+K280Y+S302T+E390A+G475K, H1*+G7A+D118T+W140Y+R180*+S181*+T182S+E189P+N194F+L205Y+Y242F+E259G+M260D+N279S+K280Y+G303R+N394W+G475K, H1*+G7A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+Y242F+T256D+N279S+K280Y+G303R+E390A+G475K, H1*+G7A+R37A+D118T+W140Y+R180*+S181*+T182S+E189P+N194F+L205Y+T224Y+Y242F+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+E259G+N279S+K280Y+S302T+E390A+N470M+G475K, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+S132L+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+H420A+G476A, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302Y+E390A+G475K, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302T+E390A+G475K, H1*+G7A+D118T+S132D+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+S302Y+N394D+G475K H1*+G7A+D118T+S132L+W140Y+R180*+S181*+T182S+E189P+N194F+L205Y+Y242F+E259G+N279S+K280Y+S302Y+N394W+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+H420A+G476H, H1*+G7A+D118T+W140Y+R180*+S181*+T182S+E189P+N194F+L205Y+T224F+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+S302Y+E390A+G475K, H1*+G7A+D118T+Q138V+W140Y+R180*+S181*+T182S+E189P+N194F+L205Y+Y242F+E259G+N279S+K280Y+S302Y+E390A+G475K, H1*+G7A+D118T+S132L+W140Y+R180*+S181*+T182S+E189P+N194F+L205Y+Y242F+E259G+N279S+K280Y+S302Y+N394W+G475K, H1*+G7A+Q98L+D118T+W140Y+R180*+S181*+T182S+E189P+N194F+L205Y+Y242F+E259G+N279S+K280Y+G303R+E390A+G475K, H1*+G7A+R37A+D118T+W140Y+R180*+S181*+T182S+E189P+N194F+L205Y+Y242F+E259G+N279S+K280Y+G303R+E390A+G475K, H1*+N3D+G7A+D118T+W140Y+R180*+S181*+T182D+E189P+N194F+Y242F+E259G+N279S+K280Y+S302T+E390A+H420A+G475K, H1*+G7A+D118T+W140Y+R180*+S181*+T182S+A185Q+E189P+N194F+L205Y+T224Y+Y242F+N279S+K280Y+G303R+E390A+G476A, H1*+G7A+D118T+W140Y+R180*+S181*+T182S+A185Q+E189P+N194F+Y242F+E259G+M260D+N279S+K280Y+S302T+E390A+G475K, H1*+G7A+R37N+D118T+W140Y+R180*+S181*+T182S+A185Q+E189P+N194F+Y242F+E259G+N279S+K280Y+S302Y+E390A+G475K, H1*+G7A+D118T+W140Y+R180*+S181*+T182S+E189P+N194F+Y242F+E259G+M260D+F266Y+N279S+K280Y+S302T+E390A+G475K, H1*+G7A+D118T+W140Y+R180*+S181*+T182S+E189P+N194F+Y242F+E259G+M260W+F266Y+N279S+K280Y+G303R+E390A+G476A, H1*+G7A+D118T+W140Y+R180*+S181*+T182D+E189P+N194F+Y242F+E259G+M260D+F266Y+N279S+K280Y+S302T+E390A+G475K, H1*+G7A+R37N+D118T+W140Y+R180*+S181*+T182D+E189P+N194F+Y242F+E259G+F266Y+N279S+K280Y+S302Y+E390A+G475K, H1*+G7A+D118T+W140Y+R180*+S181*+T182S+A185Q+E189P+N194F+T224Y+Y242F+F266Y+N279S+K280Y+G303R+E390A+G476A, H1*+G7A+D118T+W140Y+R180*+S181*+T182S+A185Q+E189P+N194F+Y242F+E259G+F266Y+N279S+K280Y+S302T+E390A+G475K, H1*+G7A+D118T+W140Y+R180*+S181*+T182S+A185Q+E189P+N194F+Y242F+E259G+F266Y+N279S+K280Y+G303R+E390A+G476A, H1*+G7A+D118T+W140Y+R180*+S181*+T182S+E189P+N194F+Y242F+E259G+M260D+N279S+K280Y+S302Y+E390A+N470M+G475K, H1*+G7A+D118T+W140Y+R180*+S181*+T182D+E189P+N194F+Y242F+E259G+M260D+N279S+K280Y+S302Y+E390A+N470M+G475K, H1*+G7A+D118T+W140Y+R180*+S181*+T182S+A185Q+E189P+N194F+Y242F+E259G+M260W+N279S+K280Y+G303R+E390A+G476A, H1*+G7A+D118T+W140Y+R180*+S181*+T182D+E189P+N194F+Y242F+E259G+M260D+N279S+K280Y+S302Y+E390A+N470M+G475K, H1*+G7A+D118T+W140Y+R180*+S181*+T182D+A185Q+E189P+N194F+Y242F+E259G+F266Y+N279S+K280Y+G303R+E390A+G475K, H1*+G7A+D118T+W140Y+R180*+S181*+T182S+A185Q+E189P+N196I+Y242F+E259G+F266Y+N279S+K280Y+G303R+E390A+G475K, H1*+G7A+D118T+W140Y+R180*+S181*+T182S+A185D+E189P+Y242F+E259G+F266Y+N279S+K280Y+G303R+E390A+H420Y+G475K, H1*+G7A+D118T+W140Y+R180*+S181*+A185Q+E189P+N194F+L205Y+Y242F+E259G+F266Y+N279S+K280Y+G303R+E390A+G475K, H1*+G7A+D118T+W140Y+R180*+S181*+T182S+E189P+N194F+Y242F+E259G+M260D+N279S+K280Y+S302Y+E390A+N470M+G475K, H1*+G7A+D118T+W140Y+R180*+S181*+T182S+A185Q+E189P+N194F+Y242F+E259G+M260W+N279S+K280Y+G303R+E390A+G476A and H1*+G7A+D118T+W140Y+R180*+S181*+T182D+E189P+N194F+L205Y+T224Y+Y242F+F266Y+N279S+K280Y+G303R+E390A+G476A, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to H1*+N3D+G7A+D118T+W140Y+R180*+S181*+T182S+E189P+N194F+L205Y+Y242F+E259G+N279S+K280Y+S302T+V365I+E390A+N474L, H1*+N3D+G7A+D118T+W140Y+R180*+S181*+T182S+E189P+N194F+L205Y+Y242F+E259G+N279S+K280Y+S302T+E390A+H420A+G475K, H1*+N3D+G7A+D118T+W140Y+R180*+S181*+T182S+E189P+N194F+L205Y+Y242F+E259G+N279S+K280Y+S302T+E390A+H420A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+R37A+D118T+W140Y+R180*+S181*+T182S+E189P+N194F+L205Y+Y242F+E259G+N279S+K280Y+G303R+N394D+N474F, H1*+N3D+G7A+D118T+W140Y+R180*+S181*+T182S+E189P+N194F+L205Y+Y242F+E259G+N279S+K280Y+S302Y+E390A+H420A+G475K, H1*+N3D+G7A+D118T+W140Y+R180*+S181*+T182S+E189P+N194F+L205Y+Y242F+E259G+N279S+K280Y+G303R+E390D+H420P+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+D118T+W140Y+R180*+S181*+T182S+E189P+N194F+L205Y+Y242F+E259G+N279S+K280Y+G303R+N394W+H420A+G475K, H1*+G7A+R37N+D118T+W140Y+R180*+S181*+T182S+E189P+N194F+L205Y+Y242F+E259G+N279S+K280Y+G303R+E390A+H420A+G475K, H1*+N3D+G7A+D118T+W140Y+R180*+S181*+T182S+E189P+N194F+L205Y+Y242F+E259G+N279S+K280Y+G303R+E390A+H420A+G475K, H1*+N3D+G7A+D118T+W140Y+R180*+S181*+T182S+E189P+N194F+L205Y+Y242F+E259G+N279S+K280Y+G303R+E390A+H420A+N474L, H1*+G7A+R37N+D118T+W140Y+R180*+S181*+T182S+E189P+N194F+L205Y+Y242F+E259G+N279S+K280Y+G303R+N394W+H420A+G475K, H1*+N3D+G7A+D118T+W140Y+R180*+S181*+T182S+E189P+N194F+L205Y+Y242F+E259G+N279S+K280Y+G303R+E390A+H420P+G475K, H1*+G7A+D118T+Q138V+W140Y+R180*+S181*+T182S+E189P+N194F+L205Y+Y242F+E259G+N279S+K280Y+G303R+H323W+E390A+N474L, H1*+G7A+D118T+W140Y+R180*+S181*+T182S+E189P+N194F+L205Y+T224F+Y242F+E259G+N279S+K280Y+G303R+N394W+H420A+G475K, H1*+G7A+D118T+W140Y+R180*+S181*+T182S+E189P+N194F+L205Y+Y242F+E259G+N279S+K280Y+G303R+P321H+Q360Y+E390A+G476A, H1*+G7A+D118T+W140Y+R180*+S181*+T182S+E189P+N194F+L205Y+Y242F+E259G+M260D+N279S+K280Y+S302Y+E390A+N470M+G475K, H1*+N3D+G7A+D118T+W140Y+R180*+S181*+T182S+E189P+N194F+L205Y+Y242F+E259G+N279S+K280Y+S302T+E390A+H420A+G475K, H1*+G7A+D118T+S132L+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+A185Q+E189P+N194F+L205Y+Y242F+E259G+F266Y+N279S+K280Y+G303R+E390A+G475K, H1*+G7A+D118T+W140Y+R180*+S181*+T182S+A185Q+E189P+N194F+L205N+Y242F+E259G+F266Y+N279S+K280Y+G303R+E390A+G475K, H1*+G7A+D118T+W140Y+R180Q+S181*+T182*+A185Q+E189P+N194F+L205Y+Y242F+E259G+F266Y+N279S+K280Y+G303R+E390A+G475K, H1*+N3D+G7A+D118T+W140Y+R180*+S181*+T182S+E189P+N194F+L205Y+Y242F+E259G+N279S+K280Y+S302T+E390A+H420A+G475K, H1*+G7A+Q98L+D118T+W140Y+R180*+S181*+T182S+E189P+N194F+L205Y+Y242F+E259G+N279S+K280Y+G303R+N394D+H420A+G475K, H1*+G7A+D118T+W140Y+R180*+S181*+T182S+E189P+N194F+L205Y+Y242F+E259G+M260D+N279S+K280Y+G303R+Y362V+E390A+G475K, H1*+G7A+D118T+Y135E+W140Y+R180*+S181*+T182S+E189P+N194F+L205Y+T224F+Y242F+E259G+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+Y371N+E390A+G475K, H1*+G7A+D118T+Y135E+W140Y+R180*+S181*+T182S+E189P+N194F+L205Y+Y242F+E259G+N279S+K280Y+G303R+P321Y+E390A+G475K, H1*+G7A+D118T+S132D+W140Y+R180*+S181*+T182S+E189P+N194F+L205Y+Y242F+E259G+N279S+K280Y+G303R+P321Y+E390A+G475K, H1*+G7A+D118T+S132D+W140Y+R180*+S181*+T182S+E189P+N194F+L205Y+Y242F+E259G+N279S+K280Y+G303R+Y362V+E390A+N474L, H1*+G7A+D118T+W140Y+R180*+S181*+T182S+E189P+N194F+L205Y+Y242F+E259G+M260D+N279S+K280Y+G303R+E390A+H420A+G475K, H1*+G7A+D118T+Y135E+W140Y+R180*+S181*+T182S+E189P+N194F+L205Y+Y242F+E259G+N279S+K280Y+G303R+N394W+H420A+G475K, H1*+N3D+G7A+D118T+W140Y+R180*+S181*+T182S+E189P+N194F+L205Y+Y242F+E259G+N279S+K280Y+G303R+N394D+H420A+G475K, H1*+G7A+D118T+Q138V+W140Y+R180*+S181*+T182S+E189P+N194F+L205Y+Y242F+E259G+N279S+K280Y+G303R+E390A+H420Q+G475K, H1*+G7A+R37N+D118T+W140Y+R180*+S181*+T182S+E189P+N194F+L205Y+Y242F+E259G+N279S+K280Y+G303R+E390A+H420P+G476H, H1*+G7A+R37A+Q98L+D118T+W140Y+R180*+S181*+T182S+E189P+N194F+L205Y+Y242F+E259G+N279S+K280Y+S302Y+E390A+G475K, H1*+G7A+R37A+D118T+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+M260D+N279S+K280Y+G303R+E390A+H420P+G476A, H1*+G7A+R37A+D118T+W140Y+R180*+S181*+T182S+E189P+N194F+L205Y+Y242F+E259G+N279S+K280Y+S302Y+E390A+H420A+G475K, H1*+G7A+D118T+Y135E+W140Y+R180*+S181*+T182S+E189P+N194F+L205Y+Y242F+E259G+N279S+K280Y+S302T+Y371N+E390A+G475K, H1*+G7A+D118T+W140Y+R180*+S181*+T182S+E189P+N194F+L205Y+Y242F+E259G+N279S+K280Y+G303R+H323W+E390A+H420A+N474L, H1*+G7A+D118T+Y135E+W140Y+R180*+S181*+T182S+E189P+N194F+L205Y+Y242F+E259G+N279S+K280Y+G303R+E390A+R414M+G476H, H1*+G7A+R37N+D118T+W140Y+R180*+S181*+T182S+E189P+N194F+L205Y+Y242F+E259G+N279S+K280Y+S302Y+E390A+H420A+G475K, H1*+G7A+R37A+D118T+W140Y+R180*+S181*+T182S+E189P+N194F+L205Y+Y242F+E259G+N279S+K280Y+S302T+E390A+H420A+G476A, H1*+G7A+R37N+D118T+W140Y+R180*+S181*+T182S+E189P+N194F+L205Y+Y242F+E259G+N279S+K280Y+S302Y+E390A+H420A+G475K, H1*+G7A+R37A+D118T+W140Y+R180*+S181*+T182S+E189P+N194F+L205Y+Y242F+E259G+N279S+K280Y+S302Y+E390A+H420A+G476H, H1*+N3D+G7A+D118T+W140Y+R180*+S181*+T182S+E189P+N194F+L205Y+Y242F+E259 G+N279S+K280Y+S302Y+N394D+H420A+G475K, H1*+G7A+R37N+D118T+W140Y+R180*+S181*+T182S+E189P+N194F+L205Y+Y242F+E259G+N279S+K280Y+S302T+E390A+H420A+G475K, H1*+G7A+R37A+D118T+W140Y+R180*+S181*+T182S+E189P+N194F+L205Y+Y242F+E259G+N279S+K280Y+S302Y+E390A+H420A+G475K, H1*+G7A+R37N+D118T+W140Y+R180*+S181*+T182S+E189P+N194F+L205Y+Y242F+E259G+N279S+K280Y+S302T+E390A+H420A+G475K, H1*+G7A+R37A+D118T+W140Y+R180*+S181*+T182S+E189P+N194F+L205Y+Y242F+E259G+N279S+K280Y+S302T+E390A+H420A+G475K, H1*+G7A+R37N+D118T+W140Y+R180*+S181*+T182S+E189P+N194F+L205Y+Y242F+E259G+N279S+K280Y+S302Y+E390A+H420A+G476H, H1*+G7A+R37N+D118T+W140Y+R180*+S181*+T182S+E189P+N194F+L205Y+Y242F+E259G+N279S+K280Y+S302T+N394D+H420A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+R37A+D118T+W140Y+R180*+S181*+T182S+E189P+N194F+L205Y+Y242F+E259G+N279S+K280Y+G303R+E390A+G475K, H1*+G7A+R37A+D118T+W140Y+R180*+S181*+T182S+E189P+N194F+L205Y+Y242F+E259G+M260D+N279S+K280Y+G303R+N394W+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+Y242F+E259G+M260D+N279S+K280Y+G303R+N394D+G475K, H1*+G7A+R37N+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+A185Q+E189P+N194F+L205Q+Y242F+E259G+F266Y+N279S+K280Y+G303R+E390A+G475K, H1*+N3G+G7A+V104M+D118T+W140Y+R180*+S181*+T182S+E189P+N194F+L205Y+Y242F+E259G+N279S+K280Y+S302Y+N394W+G475K, H1*+N3G+G7A+D118T+W140Y+R180*+S181*+T182S+E189P+N194F+L205Y+Y242F+E259G+N279S+K280Y+G303R+N394W+H420A+G475K, H1*+N3D+G7A+D118T+W140Y+R180*+S181*+T182S+E189P+N194F+L205Y+Y242F+E259G+N279S+K280Y+G303R+E390A+H420A+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+E390A+N470M+G475K, H1*+G7A+R37A+D118T+W140Y+R180*+S181*+T182S+E189P+N194F+L205Y+Y242F+E259G+M260D+N279S+K280Y+G303R+N394W+G475K, H1*+G7A+R37A+D118T+W140Y+R180*+S181*+T182S+E189P+N194F+L205Y+Y242F+E259G+N279S+K280Y+G303R+E390A+H420A+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+R37A+D118T+W140Y+R180*+S181*+T182S+E189P+N194F+L205Y+T224Y+Y242F+E259G+N279S+K280Y+G303R+N394W+G475K, H1*+G7A+R37A+D118T+W140Y+R180*+S181*+T182S+E189P+N194F+L205Y+Y242F+E259G+F261P+N279S+K280Y+G303R+E390A+G475K, H1*+G7A+R37A+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+T224Y+Y242F+E259G+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+H420A+G475K, H1*+N3D+G7A+D118T+W140Y+R180*+S181*+T182S+E189P+N194F+L205Y+Y242F+E259G+M260D+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3G+G7A+R37N+D118T+W140Y+R180*+S181*+T182S+E189P+N194F+L205Y+Y242F+E259G+N279S+K280Y+G303R+E390A+G475K, H1*+G7A+D118T+W140Y+R180*+S181*+T182S+E189P+N194F+L205Y+T224F+Y242F+E259G+N279S+K280Y+G303R+E390A+H420A+G475K, H1*+G7A+D118T+S132L+W140Y+R180*+S181*+T182S+E189P+N194F+L205Y+T224F+Y242F+E259G+N279S+K280Y+S302Y+E390A+G475K, H1*+G7A+D118T+Q138V+W140Y+R180*+S181*+T182S+E189P+N194F+L205Y+T224F+Y242F+E259G+N279S+K280Y+S302Y+E390A+G475K, H1*+G7A+R37N+D118T+W140Y+R180*+S181*+T182S+E189P+N194F+L205Y+Y242F+T256D+E259G+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+N394D+G475K, N3G+G7A+R37V+D118T+W140M+R180*+S181*+T182S+E189P+N194F+L205Y+Y242F+E259G+N279S+K280Y+G303R+E390A+H420A+G475K, H1*+G7A+R37N+D118T+W140Y+R180*+S181*+T182S+E189P+N194F+L205Y+Y242F+E259G+N279S+K280Y+G303R+H323W+N394D+G475K, H1*+N3D+G7A+D118T+W140Y+R180*+S181*+T182S+E189P+N194F+L205Y+Y242F+E259G+N279S+K280Y+G303R+H323W+E390A+G475K, H1*+N3D+G7A+D118T+W140Y+R180*+S181*+T182S+E189P+N194F+L205Y+Y242F+E259G+N279S+K280Y+G303R+H323W+E390A+G475K, H1*+G7A+D118T+S132L+W140Y+R180*+S181*+T182D+E189P+N194F+L205Y+Y242F+E259G+M260D+N279S+K280Y+G303R+E390A+G475K, H1*+G7A+D118T+W140Y+R180*+S181*+T182S+A185Q+E189P+N194F+Y242F+E259G+M260D+N279S+K280Y+S302Y+E390A+N470M+G475K, H1*+N3D+G7A+D118T+W140Y+R180*+S181*+T182S+A185Q+E189P+N194F+Y242F+E259G+N279S+K280Y+S302T+E390A+H420A+G475K, H1*+G7A+D118T+W140Y+R180*+S181*+T182S+A185Q+E189P+N194F+L205Y+Y242F+E259G+M260W+N279S+K280Y+G303R+E390A+G476A, H1*+G7A+D118T+W140Y+R180*+S181*+T182S+E189P+N194F+Y242F+E259G+M260D+F266Y+N279S+K280Y+S302Y+E390A+N470M+G475K, H1*+G7A+D118T+S132L+W140Y+R180*+S181*+T182S+E189P+N194F+Y242F+E259G+M260D+F266Y+N279S+K280Y+G303R+E390A+G475K, H1*+N3D+G7A+D118T+W140Y+R180*+S181*+T182D+E189P+N194F+Y242F+E259G+F266Y+N279S+K280Y+S302T+E390A+H420A+G475K, H1*+G7A+D118T+W140Y+R180*+S181*+T182S+A185Q+E189P+N194F+Y242F+E259G+M260W+F266Y+N279S+K280Y+G303R+E390A+G476A, H1*+G7A+R37N+D118T+W140Y+R180*+S181*+T182S+A185Q+E189P+N194F+L205Y+Y242F+E259G+N279S+K280Y+S302Y+E390A+G475K, H1*+G7A+D118T+W140Y+R180*+S181*+T182S+A185Q+E189P+N194F+L205Y+Y242F+E259G+M260D+N279S+K280Y+S302T+E390A+G475K and H1*+N3D+G7A+D118T+W140Y+R180*+S181*+T182D+E189P+N194F+L205Y+Y242F+E259G+N279S+K280Y+S302T+E390A+H420A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to H1*+G7A+N106D+A109G+F116W+D123N+N126D+T131I+T165V+N174S+R180*+S181*+T182I+F202Y+Y242F+E259G+N279S+G303R+E390A+G475K, H1*+N3D+G7A+D118T+W140Y+R180*+S181*+T182S+E189P+N194F+L205Y+Y242F+E259G+N279S+K280Y+G303R+Y362V+E390A+H420A+N474L, H1*+N3D+G7A+K93R+D118T+W140Y+R180*+S181*+T182S+E189P+N194F+L205Y+Y242F+E259G+N279S+K280Y+G303R+E390A+H420A+N474L, H1*+N3D+G7A+R37N+D118T+Q138V+W140Y+R180*+S181*+T182S+E189P+N194F+L205Y+Y242F+E259G+N279S+K280Y+G303R+E390A+G475K, H1*+G7A+D118T+Y135E+W140Y+R180*+S181*+T182S+E189P+N194F+L205Y+Y242F+E259G+M260D+N279S+K280Y+G303R+W346N+E390A+G475K, H1*+G7A+D118T+Y135E+W140Y+R180*+S181*+T182S+E189P+N194F+L205Y+Y242F+E259G+F261P+N279S+K280Y+G303R+Y362V+E390A+G475K, H1*+N3G+G7A+D118T+Q138V+W140Y+R180*+S181*+T182S+E189P+N194F+L205Y+Y242F+E259G+N279S+K280Y+G303R+E390A+H420A+G476A, H1*+G7A+R37N+D118T+S132D+W140Y+R180*+S181*+T182S+E189P+N194F+L205Y+Y242F+E259G+N279S+K280Y+G303R+E390A+N470M+G475K, H1*+N3D+G7A+R37A+D118T+W140Y+R180*+S181*+T182S+E189P+N194F+L205Y+Y242F+E259G+N279S+K280Y+S302Y+N394D+H420A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3G+G7A+R37N+D118T+W140Y+R180*+S181*+T182S+E189P+N194F+L205Y+Y242F+E259G+N279S+K280Y+G303R+N394W+H420A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3D+G7A+R37N+D118T+W140Y+R180*+S181*+T182S+E189P+N194F+L205Y+Y242F+E259G+N279S+K280Y+G303R+G361M+H420A+G475K, H1*+N3D+G7A+R37A+D118T+W140Y+R180*+S181*+T182S+E189P+N194F+L205Y+Y242F+E259G+N279S+K280Y+G303R+E390A+H420A+G475K, H1*+N3D+G7A+D118T+S132D+W140Y+R180*+S181*+T182S+E189P+N194F+L205Y+Y242F+E259G+N279S+K280Y+G303R+E390A+H420A+G475K, H1*+G7A+R37A+D118T+W140Y+R180*+S181*+T182S+E189P+N194F+L205Y+Y242F+E259G+M260W+N279S+K280Y+G303R+E390A+N470M+G475K, H1*+G7A+R37N+D118T+W140Y+R180*+S181*+T182S+E189P+N194F+L205Y+Y242F+E259G+M260W+N279S+K280Y+G303R+E390A+H420A+G475K, H1*+G7A+R37N+D118T+W140Y+R180*+S181*+T182S+E189P+N194F+L205Y+Y242F+E259G+M260D+N279S+K280Y+G303R+N394W+H420A+G475K, H1*+G7A+R37A+D118T+W140Y+R180*+S181*+T182S+E189P+N194F+L205Y+Y242F+E259G+M260D+N279S+K280Y+G303R+E390A+N470M+G475K, H1*+G7A+R37A+D118T+W140Y+R180*+S181*+T182S+E189P+N194F+L205Y+Y242F+E259G+M260D+N279S+K280Y+G303R+N394D+H420A+G475K, H1*+G7A+R37N+D118T+W140Y+N174K+R180*+S181*+T182S+E189P+N194F+L205Y+Y242F+E259G+M260D+N279S+K280Y+G303R+N394W+G475K, H1*+G7A+R37A+D118T+W140Y+R180*+S181*+T182S+E189P+N194F+L205Y+Y242F+E259G+N279S+K280Y+G303R+P321Y+L354V+N394D+G475K, H1*+G7A+R37N+D118T+W140Y+R180*+S181*+T182S+E189P+N194F+L205Y+Y242F+E259G+N279S+K280Y+G303R+H323W+Q360M+E390A+G475K, H1*+N3D+G7A+D118T+W140Y+R180*+S181*+T182S+A185Q+E189P+N194F+Y242F+E259G+F266Y+N279S+K280Y+S302T+E390A+H420A+G475K and H1*+G7A+A31S+R37N+D118T+W140Y+R180*+S181*+T182S+A185Q+E189P+N194F+Y242F+E259G+F266Y+N279S+K280Y+S302Y+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to H1*+G7A+D118T+S132L+W140Y+R180*+S181*+T182S+A185Q+E189P+N194F+L205Y+Y242F+E259G+M260D+F266Y+N279S+K280Y+G303R+E390A+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to

H1*+N54S+V56T+K72R+G113Q+F116Q+D118Q+D123N+N126D+T131 I+K142G+D144N+W167F+K172G+N174S+S181T+T182*+G183*+F202Y+E390A+F395Y+K457R+S472R+V473C+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

In one aspect, the alpha-amylase variant of SEQ ID NO: 3 comprising modifications in the positions corresponding to

H1*+N54S+V56T+K72R+N106D+A109G+G113Q+F116Q+D118Q+D123N+N126D+T131I+K142G+D144N+W167F+K172G+N174S+S181T+T182*+G183*+F202Y+E390A+F395Y+K457R+S472R+V473C+G475K, using SEQ ID NO: 1 for numbering and wherein said variant has alpha-amylase activity and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14

The variants may further comprise one or more additional substitution and/or deletion at one or more (e.g., several) other positions.

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Therefore even though the alpha-amylase variants useful in the invention may only comprise one specific substitution providing the improved property according to the invention it may still have addition modifications leading to an alpha-amylase variant having at least 80%, at least 85%, at least 90%, at least 91%, at least 92%, at least 93%, at least 94%, at least 95%, at least 96%, at least 97%, at least 98%, or at least 99%, but less than 100% sequence identity, to the amino acid sequence of the SEQ ID No: 1, 2, or 3. These additional modifications should preferably not significantly change the improved properties of the alpha-amylase variant.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for protease activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64.

In one aspect, the alpha-amylase variants useful in the present invention have an improved property relative to the parent polypeptide, wherein the improved property is selected from the group consisting of increased catalytic efficiency, increased catalytic rate, increased chemical stability, increased oxidation stability, increased pH activity, increased pH stability, increased specific activity, increased stability under storage conditions, increased substrate binding, increased substrate cleavage, increased substrate specificity, increased substrate stability, increased surface properties, increased thermal activity, and increased thermostability.

In an embodiment, the alpha-amylase variant(s) has improved (increased) specific activity compared to the parent polypeptide.

In an embodiment, the alpha-amylase variant(s) has improved (increased) wash performance compared to the parent polypeptide.

In an embodiment, the alpha-amylase variant(s) has improved (increased) wash performance compared to SEQ ID NO: 1, SEQ ID NO: 2 or SEQ ID NO: 3.

In one aspect, the alpha-amylase variant(s) has improved property (increased) relative to said parent polypeptide, wherein said improved property is improved wash performance for a measure as increased specific activity and wherein said increased specific activity in Model A detergent composition.

In one aspect, the alpha-amylase variant(s) has improved property (increased) relative to said parent polypeptide, wherein said improved property is improved wash performance for a measure of increased specific activity and wherein said increased specific activity in Model A detergent composition in micro-swatch assay and/or AMSA assay as described in examples.

In one aspect, the alpha-amylase variant(s) has improved wash performance for a measure of increased specific activity in Model A detergent composition measured as Improvement Factor (IF) is >1.0, at least 1.1, at least 1.2, at least 1.3, at least 1.4, at least 1.5, at least 1.6, at least 1.7, at least 1.8, at least 1.9, at least 2.0, at least 2.5, at least 3.0, at least 3.5, at least 4.0, at least 4.5, at least 5.0, at least 5.5, at least 6.0, at least 6.5, at least 7.0, at least 7.5, at least 8.0, at least 8.5, at least 9.0, at least 9.5, at least 10.0 relative to said parent polypeptide.

In one aspect, the alpha-amylase variant(s) has improved wash performance for a measure of increased specific activity in Model A detergent composition measured as Improvement Factor (IF) is >1.0, at least 1.1, at least 1.2, at least 1.3, at least 1.4, at least 1.5, at least 1.6, at least 1.7, at least 1.8, at least 1.9, at least 2.0, at least 2.5, at least 3.0, at least 3.5, at least 4.0, at least 4.5, at least 5.0, at least 5.5, at least 6.0, at least 6.5, at least 7.0, at least 7.5, at least 8.0, at least 8.5, at least 9.0, at least 9.5, at least 10.0 relative to SEQ ID NO: 1, SEQ ID NO: 2 or SEQ ID NO: 3.

In one aspect, the alpha-amylase variant(s) has improved wash performance for a measure of increased specific activity in Model A detergent composition measured as Improvement Factor (IF) is >1.0, at least 1.1, at least 1.2, at least 1.3, at least 1.4, at least 1.5, at least 1.6, at least 1.7, at least 1.8, at least 1.9, at least 2.0, at least 2.5, at least 3.0, at least 3.5, at least 4.0, at least 4.5, at least 5.0, at least 5.5, at least 6.0, at least 6.5, at least 7.0, at least 7.5, at least 8.0, at least 8.5, at least 9.0, at least 9.5, at least 10.0 relative to SEQ ID NO: 1, SEQ ID NO: 2 or SEQ ID NO: 3.

In one aspect, the alpha-amylase variant comprising a modification at a position corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475, 476, using SEQ ID NO: 1 for numbering, and wherein said alpha-amylase variant has improved wash performance for a measure as increased specific activity of Improvement Factor (IF) of >1.1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14 and wherein the variant has alpha-amylase activity.

In one aspect, the alpha-amylase variant comprising a modification at a position corresponding to positions: H1, N3, G7, R26, A31, R37, T40, A41, N54, V56, A60, L63, K72, G73, K93, N94, Q98, V103, V104, M105, N106, A109, G110, A111, G113, T114, F116, V117, D118, D123, N126, N128, T131, S132, T134, Y135, Q136, I137, Q138, W140, K142, D144, P146, T151, Y152, F155, W159, Y160, T165, W167, E169, K172, L173, N174, I176, Y177, K178, R180, S181, T182, G183, A185, W188, E189, E193, N194, N196, F202, A203, L205, T224, N225, Y242, S243, F244, V252, T256, K258, E259, M260, F261, F266, N276, N279, K280, W283, S286, S302, G303, T315, H320, P321, M322, H323, W346, L354, E359, Q360, G361, Y362, V365, Y371, A383, E390, N394, F395, D405, I409, R414, H420, N422, T427, G455, K457, P458, N470, S472, V473, N474, G475, G476, using SEQ ID NO: 1 for numbering, and wherein said alpha-amylase variant has improved wash performance for a measure as increased specific activity measured as Improvement Factor (IF) of >1.1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14 and wherein the variant has alpha-amylase activity.

In one aspect, the alpha-amylase variant comprising a modification at a position corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, W140S, W140M, W140Y, K142E, K142G, K142P, D144N, P146S, T151A, Y152L, F155W, W159H, W159L, Y160P, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, Y242F, Y242Y, S243Q, F244I, F244L, V252I, T256A, T256D, K258V, E259G, E259V, M260D, M260W, F261K, F261P, F261S, N279S, N280F, N280W, N280Y, W283N, S286K, S286L, S302T, S302Y, G303R, T315A, H320T, P321H, P321Y, M322G, M322Y, H323A, H323N, H323W, W346N, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering, and wherein said alpha-amylase variant has improved wash performance for a measure as increased specific activity of Improvement Factor (IF) of >1.1 and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14 and wherein the variant has alpha-amylase activity.

In one aspect, the alpha-amylase variant comprising a modification at a position corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475, 476, using SEQ ID NO: 1 for numbering, and wherein said alpha-amylase variant has improved wash performance for a measure as increased specific activity of Improvement Factor (IF) of >1.1 in Model A detergent composition and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14 and wherein the variant has alpha-amylase activity.

In one aspect, the alpha-amylase variant comprising a modification at a position corresponding to positions: H1, H2, N3, G7, R26, A31, R37, T40, A41, N54, V56, A60, L63, K72, G73, K93, N94, Q98, V103, V104, M105, N106, A109, G110, A111, G113, T114, F116, V117, D118, D123, N126, N128, T131, S132, T134, Y135, Q136, I137, Q138, W140, K142, D144, P146, T151, Y152, F155, W159, Y160, T165, W167, E169, K172, L173, N174, I176, Y177, K178, R180, S181, T182, G183, A185, W188, E189, E193, N194, N196, F202, A203, L205, T224, N225, Y242, S243, F244, V252, T256, K258, E259, M260, F261, F266, N276, N279, K280, W283, S286, S302, G303, T315, H320, P321, M322, H323, W346, L354, E359, Q360, G361, Y362, V365, Y371, A383, E390, N394, F395, D405, I409, R414, H420, N422, T427, G455, K457, P458, N470, S472, V473, N474, G475, G476, using SEQ ID NO: 1 for numbering, and wherein said alpha-amylase variant has improved wash performance for a measure as increased specific activity of Improvement Factor (IF) of >1.1 in Model A detergent composition and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14 and wherein the variant has alpha-amylase activity.

In one aspect, the alpha-amylase variant comprising a modification at a position corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, W140S, W140M, W140Y, K142E, K142G, K142P, D144N, P146S, T151A, Y152L, F155W, W159H, W159L, Y160P, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, Y242F, Y242Y, S243Q, F244I, F244L, V252I, T256A, T256D, K258V, E259G, E259V, M260D, M260W, F261K, F261P, F261S, N279S, N280F, N280W, N280Y, W283N, S286K, S286L, S302T, S302Y, G303R, T315A, H320T, P321H, P321Y, M322G, M322Y, H323A, H323N, H323W, W346N, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering, and wherein said alpha-amylase variant has improved wash performance for a measure as increased specific activity of Improvement Factor (IF) of >1.1 in Model A detergent composition and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14 and wherein the variant has alpha-amylase activity.

In one aspect, the alpha-amylase variant comprising a modification at a position corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475, 476, using SEQ ID NO: 1 for numbering, and wherein said alpha-amylase variant has improved wash performance for a measure as increased specific activity of Improvement Factor (IF) of >1.1 in Model A detergent composition and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14 and wherein the variant has alpha-amylase activity.

In one aspect, the alpha-amylase variant comprising a modification at a position corresponding to positions: H1, H2, N3, G7, R26, A31, R37, T40, A41, N54, V56, A60, L63, K72, G73, K93, N94, Q98, V103, V104, M105, N106, A109, G110, A111, G113, T114, F116, V117, D118, D123, N126, N128, T131, S132, T134, Y135, Q136, I137, Q138, W140, K142, D144, P146, T151, Y152, F155, W159, Y160, T165, W167, E169, K172, L173, N174, I176, Y177, K178, R180, S181, T182, G183, A185, W188, E189, E193, N194, N196, F202, A203, L205, T224, N225, Y242, S243, F244, V252, T256, K258, E259, M260, F261, F266, N276, N279, K280, W283, S286, S302, G303, T315, H320, P321, M322, H323, W346, L354, E359, Q360, G361, Y362, V365, Y371, A383, E390, N394, F395, D405, I409, R414, H420, N422, T427, G455, K457, P458, N470, S472, V473, N474, G475, G476, using SEQ ID NO: 1 for numbering, and wherein said alpha-amylase variant has improved wash performance for a measure as increased specific activity of Improvement Factor (IF) of >1.1 in Model A detergent composition and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14 and wherein the variant has alpha-amylase activity.

In one aspect, the alpha-amylase variant comprising a modification at a position corresponding to positions: H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, W140S, W140M, W140Y, K142E, K142G, K142P, D144N, P146S, T151A, Y152L, F155W, W159H, W159L, Y160P, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, Y242F, Y242Y, S243Q, F244I, F244L, V252I, T256A, T256D, K258V, E259G, E259V, M260D, M260W, F261K, F261P, F261S, N279S, N280F, N280W, N280Y, W283N, S286K, S286L, S302T, S302Y, G303R, T315A, H320T, P321H, P321Y, M322G, M322Y, H323A, H323N, H323W, W346N, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering, and wherein said alpha-amylase variant has improved wash performance for a measure as increased specific activity of Improvement Factor (IF) of >1.1 in Model A detergent composition and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14 and wherein the variant has alpha-amylase activity.

In a one aspect, the alpha-amylase variant has improved wash performance for a measure as increased specific activity of Improvement Factor (IF) of >1.1 in Model A detergent composition compared to parent polypeptide, and wherein said variant is selected from group consisting of: H1*+G7A+R180*+S181*+Y242F+E259G+N279S+G303R+E390A+G475K H1*+G7A+S181*+T182*+Y242F+E259G+N279S+G303R+E390A+G475K H1*+G7A+R180*+S181*+T182S+Y242F+E259G+N279S+G303R+E390A+G475K H1*+G7A+R180*+T182L+G183*+Y242F+E259G+N279S+G303R+E390A+G475K H1*+G7A+S181 L+T182*+G183*+Y242F+E259G+N279S+G303R+E390A+G475K H1*+G7A+R180S+T182*+G183*+Y242F+E259G+N279S+G303R+E390A+G475K H1*+G7A+A60P+R180*+S181*+Y242F+E259G+N279S+G303R+E390A+G475K H1*+G7A+M105L+R180*+S181*+Y242F+E259G+N279S+G303R+E390A+G475K H1*+G7A+M105I+R180*+S181*+Y242F+E259G+N279S+G303R+E390A+G475K H1*+G7A+R180*+S181*+T182D+Y242F+E259G+N279S+G303R+E390A+G475K H1*+G7A+R180*+S181*+T182E+Y242F+E259G+N279S+G303R+E390A+G475K H1*+G7A+R180*+S181*+T182G+Y242F+E259G+N279S+G303R+E390A+G475K H1*+G7A+R180*+S181*+T182M+Y242F+E259G+N279S+G303R+E390A+G475K H1*+G7A+R180*+S181*+A185K+Y242F+E259G+N279S+G303R+E390A+G475K H1*+G7A+R180*+S181*+A185D+Y242F+E259G+N279S+G303R+E390A+G475K H1*+G7A+R180*+S181*+A185Q+Y242F+E259G+N279S+G303R+E390A+G475K H1*+G7A+R180*+S181*+A185N+Y242F+E259G+N279S+G303R+E390A+G475K H1*+G7A+R180*+S181*+A185R+Y242F+E259G+N279S+G303R+E390A+G475K H1*+G7A+R180*+S181*+A185E+Y242F+E259G+N279S+G303R+E390A+G475K H1*+G7A+R180*+S181*+E189P+Y242F+E259G+N279S+G303R+E390A+G475K H1*+G7A+R180*+S181*+L205Y+Y242F+E259G+N279S+G303R+E390A+G475K H1*+G7A+R180*+S181*+L205F+Y242F+E259G+N279S+G303R+E390A+G475K H1*+G7A+R180*+S181*+Y242F+S243Q+E259G+N279S+G303R+E390A+G475K H1*+G7A+R180*+S181*+Y242F+F244L+E259G+N279S+G303R+E390A+G475K H1*+G7A+R180*+S181*+Y242F+F244I+E259G+N279S+G303R+E390A+G475K H1*+G7A+R180*+S181*+Y242F+E259G+F266Y+N279S+G303R+E390A+G475K H1*+G7A+R180*+S181*+N194F+Y242F+E259G+N279S+G303R+E390A+G475K H1*+G7A+R180*+S181*+N196F+Y242F+E259G+N279S+G303R+E390A+G475K H1*+G7A+R180*+S181*+N194F+L205Y+Y242F+E259G+N279S+G303R+E390A+G475K H1*+G7A+R180*+S181*+N194F+L205F+Y242F+E259G+N279S+G303R+E390A+G475K H1*+G7A+R180*+S181*+N196F+L205Y+Y242F+E259G+N279S+G303R+E390A+G475K H1*+G7A+R180*+S181*+E189P+Y242F+E259G+N276F+N279S+G303R+E390A+G475K H1*+G7A+R180*+S181*+E189P+Y242F+E259G+N276Y+N279S+G303R+E390A+G475K H1*+G7A+R180*+S181*+E189P+Y242F+E259G+N276W+N279S+G303R+E390A+G475K H1*+G7A+R180*+S181*+E189P+Y242F+E259G+N279S+K280Y+G303R+E390A+G475K H1*+G7A+R180*+S181*+E189P+Y242F+E259G+N279S+K280W+G303R+E390A+G475K H1*+G7A+R180*+S181*+E189P+L205F+Y242F+E259G+N276F+N279S+G303R+E390A+G475K H1*+G7A+R180*+S181*+E189P+L205F+Y242F+E259G+N276Y+N279S+G303R+E390A+G475K H1*+G7A+R180*+S181*+E189P+L205F+Y242F+E259G+N276W+N279S+G303R+E390A+G475K H1*+G7A+R180*+S181*+E189P+L205F+Y242F+E259G+N279S+K280F+G303R+E390A+G475K H1*+G7A+R180*+S181*+E189P+L205F+Y242F+E259G+N279S+K280Y+G303R+E390A+G475K H1*+G7A+R180*+S181*+E189P+L205F+Y242F+E259G+N279S+K280W+G303R+E390A+G475K H1*+G7A+R180*+S181*+E189P+L205Y+Y242F+E259G+N276F+N279S+G303R+E390A+G475K H1*+G7A+R180*+S181*+E189P+L205Y+Y242F+E259G+N276Y+N279S+G303R+E390A+G475K H1*+G7A+R180*+S181*+E189P+L205Y+Y242F+E259G+N276W+N279S+G303R+E390A+G475K H1*+G7A+R180*+S181*+E189P+L205Y+Y242F+E259G+N279S+K280F+G303R+E390A+G475K H1*+G7A+R180*+S181*+E189P+L205Y+Y242F+E259G+N279S+K280Y+G303R+E390A+G475K H1*+G7A+R180*+S181*+E189P+L205Y+Y242F+E259G+N279S+K280W+G303R+E390A+G475K H1*+G7A+G113S+F116W+D123N+S181*+T182*+Y242F+E259G+N279S+G303R+E390A+G475K H1*+G7A+F116L+D123N+N126D+T131I+S181*+T182*+F202Y+Y242F+E259G+N279S+G303R+E390A+G475K H1*+G7S+T40N+Q98L+R180*+S181*+Y242F+E259G+N279S+G303R+Y362V+E390A+G475K H1*+G7A+R180*+S181*+Y242F+E259G+N279S+G303R+M322G+E359W+Q360W+E390A+G475K H1*+G7A+R180*+S181*+Y242F+E259G+N279S+G303R+M322G+Q360W+E390A+G475K H1*+G7A+R180*+S181*+Y242F+E259G+N279S+G303R+E359W+Q360W+E390A+G475K H1*+G7S+R180*+S181*+Y242F+E259G+F261S+N279S+G303R+Y362V+E390A+G475K H1*+G7A+T40N+Q98L+R180*+S181*+Y242F+E259G+N279S+G303R+E390A+G475K H1*+G7S+T40N+Q98L+R180*+S181*+Y242F+E259G+N279S+G303R+E390A+G475K H1*+G7A+R180*+S181*+Y242F+E259G+F261S+N279S+S286K+G303R+Y362V+E390A+G475K H1*+G7A+R180*+S181*+Y242F+E259G+N279S+W283N+S286K+G303R+H320T+H323A+E390A+G475K H1*+G7A+R180*+S181*+Y242F+E259G+N279S+G303R+P321H+M322Y+Q360Y+E390A+G475K H1*+G7A+D123N+I176V+R180*+S181*+Y242F+E259G+N279S+G303R+E390A+G475K H1*+G7A+G113A+F116W+D123N+T131V+Q136K+E169Q+K172Q+R180*+S181*+Y242F+E259G+N279S+G303R+E390A+G475K H1*+G7A+G113S+F116W+D123N+R180*+S181*+Y242F+E259G+N279S+G303R+E390A+G475K H1*+G7A+F116L+D123N+N126D+T131I+R180*+S181*+F202Y+Y242F+E259G+N279S+G303R+E390A+G475K H1*+G7A+N106D+A109G+F116W+D123N+N126D+T131+T165V+N174S+R180*+S181*+T182I+F202Y+Y242F+E259G+N279S+G303R+E390A+G475K H1*+G7S+T40N+Q98L+R180*+S181*+Y242F+E259G+N279S+G303R+H320T+E390A+G475K H1*+G7S+T40N+Q98L+R180*+S181*+Y242F+E259G+N279S+G303R+Y362V+E390A+G475K H1*+G7A+R180*+S181*+Y242F+E259G+N279S+G303R+M322G+E390A+G475K H1*+G7A+R180*+S181*+Y242F+E259G+N279S+G303R+M322G+Q360W+E390A+G475K H1*+G7A+T40N+R180*+S181*+Y242F+E259G+N279S+G303R+E390A+G475K H1*+G7S+Q98L+R180*+S181*+Y242F+E259G+N279S+G303R+E390A+G475K H1*+G7S+R180*+S181*+Y242F+E259G+F261K+N279S+G303R+E390A+G475K H1*+G7S+R180*+S181*+Y242F+E259G+N279S+G303R+Y362V+E390A+G475K H1*+G7A+T40N+Q98L+R180*+S181*+Y242F+E259G+N279S+G303R+E390A+G475K H1*+G7A+R180*+S181*+Y242F+E259G+N279S+G303R+Y362V+E390A+G475K H1*+G7S+R180*+S181*+Y242F+E259G+F261S+N279S+G303R+E390A+G475K H1*+G7A+1137F+R180*+S181*+Y242F+E259G+F261S+N279S+G303R+Y362V+E390A+G475K H1*+G7A+R180*+S181*+Y242F+E259G+N279S+S286K+G303R+H323A+E390A+G475K H1*+G7A+R180*+S181*+Y242F+E259G+N279S+G303R+H320T+H323A+E390A+G475K H1*+G7A+R180*+S181*+Y242F+E259G+N279S+G303R+P321H+M322Y+E390A+G475K H1*+G7A+R180*+S181*+Y242F+E259G+N279S+G303R+P321H+Q360Y+E390A+G475K H1*+G7A+R180*+S181*+Y242F+E259G+N279S+G303R+M322Y+Q360Y+E390A+G475K H1*+G7S+T40N+R180*+S181*+Y242F+E259G+N279S+G303R+Y362V+E390A+G475K H1*+G7S+R180*+S181*+Y242F+E259G+F261S+N279S+G303R+Y362V+E390A+G475K H1*+G7A+R180*+S181*+Y242F+E259G+F261S+N279S+S286K+G303R+Y362V+E390A+G475K H1*+G7A+R180*+S181*+Y242F+E259G+N279S+S286L+G303R+E390A+G475K H1*+G7A+G110K+R180*+S181*+Y242F+E259G+N279S+G303R+E390A+G475K H1*+G7A+W140S+R180*+S181*+Y242F+E259G+N279S+G303R+E390A+G475K H1*+G7A+W167P+R180*+S181*+Y242F+E259G+N279S+G303R+E390A+G475K H1*+G7A+R180*+S181*+Y242F+E259G+N279S+G303R+H323A+E390A+G475K H1*+G7A+R180*+S181*+Y242F+E259G+N279S+W283N+G303R+H320T+H323A+E390A+G475K H1*+G7A+R180*+S181*+Y242F+E259G+N279S+G303R+H320T+E390A+G475K H1*+G7A+R180*+S181*+Y242F+E259G+N279S+G303R+P321H+E390A+G475K H1*+G7A+R180*+S181*+Y242F+E259G+N279S+G303R+M322Y+E390A+G475K H1*+G7A+R180*+S181*+Y242F+E259G+N279S+G303R+Q360Y+E390A+G475K H1*+G7S+T40N+Q98L+R180*+S181*+Y242F+E259G+N279S+G303R+E390A+G475K H1*+G7A+Q98L+R180*+S181*+Y242F+E259G+F261K+N279S+G303R+Y362V+E390A+G475K H1*+G7A+Q98L+R180*+S181*+Y242F+E259G+N279S+G303R+E390A+G475K H1*+G7A+R180*+S181*+Y242F+E259G+F261S+N279S+G303R+Y362V+E390A+G475K H1*+G7A+R180*+S181*+Y242F+E259G+N279S+S286L+G303R+H320T+E390A+G475K H1*+G7A+R180*+S181*+Y242F+E259G+F261S+N279S+G303R+E390A+G475K H1*+G7A+W140Y+R180*+S181*+T182D+N194F+L205Y+Y242F+E259G+N279S+G303R+E390A+G475K H1*+G7A+W140Y+R180*+S181*+T182D+N194F+L205V+Y242F+E259G+N279S+G303R+E390A+G475K H1*+G7A+D118T+W140Y+R180*+S181*+T182D+N194F+L205Y+Y242F+E259G+N279S+G303R+E390A+G475K H1*+G7A+F116N+W140Y+R180*+S181*+T182D+N194F+L205Y+Y242F+E259G+N279S+G303R+E390A+G475K H1*+G7A+W140Y+R180*+S181*+E189P+N194F+L205Y+Y242F+E259G+N279S+K280Y+G303R+E390A+G475K H1*+G7A+D118T+W140Y+R180*+S181*+E189P+N194F+L205Y+Y242F+E259G+N279S+K280Y+G303R+E390A+G475K H1*+G7A+F116N+W140Y+R180*+S181*+E189P+N194F+L205Y+Y242F+E259G+N279S+K280Y+G303R+E390A+G475K H1*+G7A+W140Y+R180*+S181*+T182D+E189P+N194F+L205Y+Y242F+E259G+N279S+G303R+E390A+G475K H1*+G7A+T134D+W140Y+R180*+S181*+E189P+N194F+L205V+Y242F+E259G+N279S+G303R+E390A+G475K H1*+G7A+F116N+W140Y+R180*+S181*+T182D+E189P+N194F+L205Y+Y242F+E259G+N279S+G303R+E390A+G475K H1*+G7A+W140Y+R180*+S181*+T182S+N194F+L205Y+Y242F+E259G+N279S+G303R+E390A+G475K H1*+G7A+W140Y+R180*+S181*+T182S+N194F+L205V+Y242F+E259G+N279S+G303R+E390A+G475K H1*+G7A+D118T+W140Y+R180*+S181*+T182S+N194F+L205Y+Y242F+E259G+N279S+G303R+E390A+G475K H1*+G7A+F116N+W140Y+R180*+S181*+T182S+N194F+L205Y+Y242F+E259G+N279S+G303R+E390A+G475K H1*+G7A+W140Y+R180*+S181*+T182S+E189P+N194F+L205Y+Y242F+E259G+N279S+G303R+E390A+G475K H1*+G7A+W140Y+R180*+S181*+T182S+E189P+N194F+L205V+Y242F+E259G+N279S+G303R+E390A+G475K H1*+G7A+F116N+W140Y+R180*+S181*+T182S+E189P+N194F+L205Y+Y242F+E259G+N279S+K280Y+G303R+E390A+G475K H1*+G7A+D123N+W140Y+R180*+S181*+N194F+L205V+Y242F+E259G+N279S+G303R+E390A+G475K H1*+G7A+D123N+W140Y+I176V+R180*+S181*+N194F+L205V+Y242F+E259G+N279S+G303R+E390A+G475K H1*+G7A+G113S+F116W+D123N+W140Y+R180*+S181*+N194F+L205V+Y242F+E259G+N279S+G303R+E390A+G475K H1*+G7A+F116L+D123N+N126D+T131I+W140Y+R180*+S181*+N194F+F202Y+L205V+Y242F+E259G+N279S+G303R+E390A+G475K H1*+G7A+N106D+A109G+W140Y+R180*+S181*+N194F+F202Y+L205V+Y242F+E259G+N279S+G303R+E390A+G475K H1*+G7A+D123N+W140Y+R180*+S181*+N194F+L205Y+Y242F+E259G+N279S+G303R+E390A+G475K H1*+G7A+D123N+W140Y+I176V+R180*+S181*+N194F+L205Y+Y242F+E259G+N279S+G303R+E390A+G475K H1*+G7A+G113S+F116W+D123N+W140Y+R180*+S181*+N194F+L205Y+Y242F+E259G+N279S+G303R+E390A+G475K H1*+G7A+F116L+D123N+N126D+T131I+W140Y+R180*+S181*+N194F+F202Y+L205Y+Y242F+E259G+N279S+G303R+E390A+G475K H1*+G7A+Q98L+G110K+P146S+R180*+S181*+Y242F+E259G+N279S+G303R+A383D+E390A+G475K H1*+N3S+G7A+Q98L+G110K+R180*+S181*+Y242F+V252+E259G+N279S+G303R+E390A+G475K H1*+G7H+Q98L+R180*+S181*+Y242F+E259G+N279S+G303R+A383D+E390A+D405G+G475K H1*+G7H+Q98L+G110K+R180*+S181*+Y242F+E259G+N279S+G303R+E390A+G475K H1*+G7A+Q98L+G110K+S132D+Y135E+R180*+S181*+Y242F+E259G+N279S+G303R+T315A+E390A+G475K H1*+G7H+G110K+R180*+S181*+Y242F+E259G+N279S+G303R+E390A+G475K H1*+A60V+Q98L+W140Y+R180*+S181*+N194F+L205Y+Y242F+E259G+N279S+G303R+A383D+E390A+G475K H1*+G7A+W140Y+T151A+R180*+S181*+N194F+L205Y+Y242F+K258V+E259V+N279S+G303R+E390A+G475K H1*+N3S+G7A+N94D+Q98L+W140Y+R180*+S181*+N194F+L205Y+Y242F+V252+E259G+N279S+G303R+E390A+G475K H1*+G7A+Q98L+G110K+R180*+S181*+Y242F+T256A+E259G+N279S+G303R+E390A+G475K H1*+G7A+Q98L+G110K+R180*+S181*+Y242F+E259G+N279S+G303R+A383D+E390A+G475K H1*+N3S+G7A+N94D+Q98L+G110K+R180*+S181*+Y242F+V252+E259G+N279S+G303R+E390A+G475K H1*+G7A+Q98L+G110K+R180*+S181*+Y242F+E259G+N279S+G303R+E390A+G475K H1*+G7H+Q98L+G110K+R180*+S181*+Y242F+E259G+N279S+G303R+E390A+G475K H1*+G7A+W140Y+T151A+R180*+S181*+N194F+L205Y+Y242F+K258V+E259V+N279S+G303R+E390A+G475K H1*+G7A+Q98L+G110K+R180*+S181*+Y242F+E259G+N279S+G303R+E390A+G475K H1*+G7A+R180*+S181*+Y242F+E259G+N279S+S286L+G303R+Y362V+E390A+G475K H1*+G7A+R180*+S181*+Y242F+E259G+N279S+W283N+S286K+G303R+E390A+G475K H1*+G7A+R180*+S181*+Y242F+E259G+N279S+W283N+G303R+H320T+E390A+G475K H1*+G7A+R180*+S181*+Y242F+E259G+N279S+W283N+G303R+H323A+E390A+G475K H1*+G7A+R180*+S181*+Y242F+E259G+N279S+S286K+G303R+H320T+E390A+G475K H1*+G7A+R180*+S181*+Y242F+E259G+N279S+S286K+G303R+Y362V+E390A+G475K H1*+G7S+R180*+S181*+Y242F+E259G+N279S+G303R+E390A+G475K H1*+G7A+Q98L+R180*+S181*+Y242F+E259G+N279S+G303R+E390A+G475K H1*+G7A+R180*+S181*+Y242F+E259G+F261K+N279S+G303R+E390A+G475K H1*+G7A+R180*+S181*+Y242F+E259G+N279S+G303R+M322G+E359W+E390A+G475K H1*+G7A+F116N+W140Y+R180*+S181*+T182D+E189P+N194F+L205Y+Y242F+E259G+N279S+K280Y+G303R+E390A+G475K H1*+G7A+F116N+W140Y+R180*+S181*+T182D+E189P+N194F+Y242F+E259G+N279S+K280Y+G303R+E390A+G475K H1*+G7A+F116N+W140Y+R180*+S181*+T182S+E189P+N194F+Y242F+E259G+N279S+K280Y+G303R+E390A+G475K H1*+G7A+F116N+W140Y+R180*+S181*+T182D+N194F+L205Y+Y242F+E259G+F266Y+N279S+G303R+E390A+G475K H1*+G7A+F116N+W140Y+R180*+S181*+A185Q+N194F+L205Y+Y242F+E259G+F266Y+N279S+G303R+E390A+G475K H1*+G7A+F116N+W140Y+R180*+S181*+T182D+A185Q+N194F+L205Y+Y242F+E259G+F266Y+N279S+G303R+E390A+G475K H1*+G7A+F116N+W140Y+R180*+S181*+N194F+L205Y+Y242F+E259G+F266Y+N279S+G303R+E390A+G475K H1*+G7A+F116N+W140Y+R180*+S181*+A185Q+N194F+L205Y+Y242F+E259G+N279S+G303R+E390A+G475K H1*+G7A+F116N+W140Y+R180*+S181*+T182D+A185Q+N194F+L205Y+Y242F+E259G+N279S+G303R+E390A+G475K H1*+G7A+D118T+W140Y+R180*+S181*+E189P+N194F+Y242F+E259G+N279S+K280Y+G303R+E390A+G475K H1*+G7A+D118T+W140Y+R180*+S181*+T182S+E189P+N194F+L205Y+Y242F+E259G+N279S+K280Y+G303R+E390A+G475K H1*+G7A+D118T+W140Y+R180*+S181*+T182D+N194F+L205Y+Y242F+E259G+F266Y+N279S+G303R+E390A+G475K H1*+G7A+D118T+W140Y+R180*+S181*+A185Q+N194F+L205Y+Y242F+E259G+F266Y+N279S+G303R+E390A+G475K H1*+G7A+D118T+W140Y+R180*+S181*+T182D+A185Q+N194F+L205Y+Y242F+E259G+F266Y+N279S+G303R+E390A+G475K H1*+G7A+D118T+W140Y+R180*+S181*+N194F+L205Y+Y242F+E259G+F266Y+N279S+G303R+E390A+G475K H1*+G7A+D118T+W140Y+R180*+S181*+A185Q+N194F+L205Y+Y242F+E259G+N279S+G303R+E390A+G475K H1*+G7A+D118T+W140Y+R180*+S181*+T182D+A185Q+N194F+L205Y+Y242F+E259G+N279S+G303R+E390A+G475K H1*+G7A+W140Y+R180*+S181*+E189P+N194F+L205V+Y242F+E259G+N279S+K280Y+G303R+E390A+G475K H1*+G7A+W140Y+R180*+S181*+T182D+E189P+N194F+L205V+Y242F+E259G+N279S+K280Y+G303R+E390A+G475K H1*+G7A+W140Y+R180*+S181*+T182D+E189P+N194F+Y242F+E259G+N279S+K280Y+G303R+E390A+G475K H1*+G7A+W140Y+R180*+S181*+T182S+E189P+N194F+Y242F+E259G+N279S+K280Y+G303R+E390A+G475K H1*+G7A+W140Y+R180*+S181*+T182D+N194F+L205V+Y242F+E259G+F266Y+N279S+G303R+E390A+G475K H1*+G7A+W140Y+R180*+S181*+A185Q+N194F+L205V+Y242F+E259G+F266Y+N279S+G303R+E390A+G475K H1*+G7A+W140Y+R180*+S181*+T182D+A185Q+N194F+L205V+Y242F+E259G+F266Y+N279S+G303R+E390A+G475K H1*+G7A+W140Y+R180*+S181*+N194F+L205V+Y242F+E259G+F266Y+N279S+G303R+E390A+G475K H1*+G7A+W140Y+R180*+S181*+A185Q+N194F+L205V+Y242F+E259G+N279S+G303R+E390A+G475K H1*+G7A+W140Y+R180*+S181*+T182D+A185Q+N194F+L205V+Y242F+E259G+N279S+G303R+E390A+G475K H1*+G7A+W140Y+R180*+S181*+T182D+E189P+N194F+L205Y+Y242F+E259G+N279S+K280Y+G303R+E390A+G475K H1*+G7A+W140Y+R180*+S181*+T182D+E189P+N194F+Y242F+E259G+N279S+K280Y+G303R+E390A+G475K H1*+G7A+W140Y+R180*+S181*+T182S+E189P+N194F+L205Y+Y242F+E259G+N279S+K280Y+G303R+E390A+G475K H1*+G7A+W140Y+R180*+S181*+T182S+E189P+N194F+Y242F+E259G+N279S+K280Y+G303R+E390A+G475K H1*+G7A+W140Y+R180*+S181*+T182D+N194F+L205Y+Y242F+E259G+F266Y+N279S+G303R+E390A+G475K H1*+G7A+W140Y+R180*+S181*+A185Q+N194F+L205Y+Y242F+E259G+F266Y+N279S+G303R+E390A+G475K H1*+G7A+W140Y+R180*+S181*+T182D+A185Q+N194F+L205Y+Y242F+E259G+F266Y+N279S+G303R+E390A+G475K H1*+G7A+W140Y+R180*+S181*+A185Q+N194F+L205Y+Y242F+E259G+N279S+G303R+E390A+G475K H1*+G7A+W140Y+R180*+S181*+T182D+A185Q+N194F+L205Y+Y242F+E259G+N279S+G303R+E390A+G475K H1*+G7A+F116N+W140Y+R180*+S181*+E189P+N194F+Y242F+E259G+N279S+K280Y+G303R+E390A+G475K H1*+G7A+D118T+W140Y+R180*+S181*+T182D+E189P+N194F+L205Y+Y242F+E259G+N279S+K280Y+G303R+E390A+G475K H1*+G7A+D118T+W140Y+R180*+S181*+T182D+E189P+N194F+Y242F+E259G+N279S+K280Y+G303R+E390A+G475K H1*+G7A+D118T+W140Y+R180*+S181*+T182S+E189P+N194F+Y242F+E259G+N279S+K280Y+G303R+E390A+G475K H1*+G7A+W140Y+R180*+S181*+E189P+N194F+Y242F+E259G+N279S+K280Y+G303R+E390A+G475K H1*+G7A+W140Y+R180*+S181*+E189P+N194F+Y242F+E259G+N279S+K280Y+G303R+E390A+G475K H1*+G7A+W140Y+R180*+S181*+N194F+L205Y+Y242F+E259G+F266Y+N279S+G303R+E390A+G475K H1*+G7A+W140Y+R180*+S181*+N194F+L205Y+Y242F+E259G+N279S+S302T+G303R+E390A+G475K H1*+G7A+W140Y+R180*+S181*+N194F+L205Y+Y242F+E259G+N279S+G303R+E390A+N474F+G475K H1*+G7A+W140Y+R180*+S181*+N194F+L205Y+Y242F+E259G+N279S+G303R+E390A+N422V+G475K H1*+G7A+W140Y+R180*+S181*+N194F+L205Y+Y242F+E259G+N279S+G303R+E390A+G476A H1*+G7A+W140Y+R180*+S181*+N194F+L205Y+Y242F+E259G+N279S+G303R+N394W+G475K H1*+G7A+W140Y+R180*+S181*+N194F+L205Y+Y242F+E259G+N279S+G303R+E390A+N474L H1*+G7A+W140Y+R180*+S181*+N194F+L205Y+Y242F+E259G+N279S+S302T+E390A+G475K H1*+G7A+W140Y+R180*+S181*+N194F+L205Y+Y242F+E259G+N279S+G303R+E390A+G475E H1*+G7A+W140Y+R180*+S181*+N194F+L205Y+Y242F+E259G+F261P+N279S+G303R+E390A+G475K H1*+G7A+W140Y+R180*+S181*+N194F+L205Y+Y242F+E259G+N279S+G303R+E390A+N474F H1*+G7A+W140Y+R180*+S181*+N194F+L205Y+Y242F+E259G+N279S+S302Y+E390A+G475K H1*+G7A+W140Y+R180*+S181*+N194F+L205Y+Y242F+E259G+N279S+G303R+E390A+N470A+G475K H1*+G7A+W140Y+R180*+S181*+N194F+L205Y+Y242F+T256D+N279S+G303R+E390A+G475K H1*+G7A+W140Y+R180*+S181*+N194F+L205Y+Y242L+E259G+N279S+G303R+W346N+E390A+G475K H1*+G7A+W140Y+R180*+S181*+N194F+L205Y+Y242F+E259G+N279S+G303R+E390A+H420P+G475K H1*+G7A+R26Y+W140Y+R180*+S181*+N194F+L205Y+Y242F+E259G+N279S+G303R+E390A+G475K H1*+G7A+R37A+W140Y+R180*+S181*+N194F+L205Y+Y242F+E259G+N279S+G303R+G475K H1*+W140Y+R180*+S181*+N194F+L205Y+Y242F+E259G+N279S+G303R+Q360Y+E390A+G475K H1*+G7A+R37N+W140Y+R180*+S181*+N194F+L205Y+Y242F+E259G+N279S+G303R+G475K H1*+W140Y+R180*+S181*+N194F+L205Y+Y242F+E259G+N279S+G303R+E390A+R414M+G475K H1*+W140Y+R180*+S181*+N194F+L205Y+Y242F+E259G+F261P+N279S+G303R+E390A+G475K H1*+W140Y+R180*+S181*+N194F+L205Y+Y242F+E259G+N279S+G303R+H323W+E390A+G475K H1*+W140Y+R180*+S181*+N194F+L205Y+Y242F+E259G+N279S+G303R+P321Y+E390A+G475K H1*+W140Y+R180*+S181*+N194F+L205Y+Y242F+E259G+N279S+G303R+Q360M+E390A+G475K H1*+G7A+W140Y+R180*+S181*+N194F+L205Y+Y242F+E259G+N279S+G303R+Y371S+E390A+G475K H1*+G7A+W140Y+R180*+S181*+T182D+A185Q+N194F+Y242F+S243Q+E259G+N279S+K280Y+G303R+E390A+G475K H1*+G7A+W140Y+R180*+S181*+T182D+Y242F+S243Q+E259G+F266Y+N279S+K280Y+G303R+E390A+G475K H1*+G7A+W140Y+R180*+S181*+A185Q+N194F+Y242F+S243Q+E259G+F266Y+N279S+K280Y+G303R+E390A+G475K H1*+G7A+W140Y+R180*+S181*+T182D+A185Q+Y242F+E259G+N279S+K280Y+G303R+E390A+G475K H1*+G7A+W140Y+R180*+S181*+T182D+E189P+Y242F+S243Q+E259G+N279S+K280Y+G303R+E390A+G475K H1*+G7A+W140Y+R180*+S181*+A185Q+Y242F+E259G+F266Y+N279S+K280Y+G303R+E390A+T427L+G475K H1*+G7A+W140Y+R180*+S181*+A185Q+E189P+Y242F+E259G+N279S+K280Y+G303R+E390A+G475K H1*+G7A+W140Y+R180*+S181*+T182D+A185Q+E189P+N194F+Y242F+S243Q+E259G+N279S+K280Y+G303R+E390A+G475K H1*+G7A+W140Y+R180*+S181*+T182D+A185Q+E189P+N194F+L205Y+Y242F+S243Q+E259G+N279S+K280Y+G303R+E390A+G475K H1*+G7A+W140Y+R180*+S181*+T182D+E189P+L205Y+Y242F+E259G+F266Y+N279S+K280Y+G303R+E390A+G475K H1*+G7A+W140Y+R180*+S181*+A185Q+E189P+N194F+Y242F+E259G+F266Y+N279S+K280Y+G303R+E390A+G475K H1*+G7A+W140Y+R180*+S181*+A185Q+E189P+N194F+Y242F+S243Q+E259G+F266Y+N279S+K280Y+G303R+E390A+G475K H1*+G7A+W140Y+R180*+S181*+A185Q+E189P+N194F+L205Y+Y242F+S243Q+E259G+F266Y+N279S+K280Y+G303R+E390A+G475K H1*+G7A+W140Y+R180*+S181*+T182D+A185Q+E189P+N194F+L205Y+Y242F+E259G+F266Y+N279S+K280Y+G303R+E390A+G475K H1*+G7A+W140Y+R180*+S181*+T182D+A185Q+E189P+N194F+Y242F+E259G+F266Y+N279S+K280Y+G303R+E390A+G475K H1*+G7A+W140Y+R180*+S181*+A185Q+Y242F+S243Q+E259G+F266Y+N279S+K280Y+G303R+E390A+G475K H1*+G7A+W140Y+R180*+S181*+T182D+A185Q+Y242F+S243Q+E259G+F266Y+N279S+G303R+E390A+G475K H1*+G7A+W140Y+R180*+S181*+Y242F+S243Q+E259G+N279S+K280Y+G303R+E390A+G475K H1*+G7A+W140Y+R180*+S181*+T182D+E189P+N194F+Y242F+E259G+N279S+K280Y+G303R+E390A+G475K H1*+G7A+W140Y+R180*+S181*+A185Q+N194F+Y242F+S243Q+E259G+N279S+K280Y+G303R+E390A+G475K H1*+G7A+W140Y+R180*+S181*+T182D+N194F+Y242F+S243Q+E259G+N279S+K280Y+G303R+E390A+G475K H1*+G7A+W140Y+R180*+S181*+A185Q+E189P+N194F+Y242F+E259G+N279S+K280Y+G303R+E390A+G475K H1*+G7A+W140Y+R180*+S181*+E189P+Y242F+S243Q+E259G+N279S+K280Y+G303R+E390A+G475K H1*+G7A+W140Y+R180*+S181*+T182D+Y242F+S243Q+E259G+N279S+K280Y+G303R+E390A+G475K H1*+G7A+W140Y+R180*+S181*+E189P+L205Y+Y242F+S243Q+E259G+N279S+K280Y+G303R+E390A+G475K H1*+G7A+W140Y+R180*+S181*+Y242F+E259G+F266Y+N279S+K280Y+G303R+E390A+G475K H1*+G7A+W140Y+R180*+S181*+T182D+A185Q+Y242F+E259G+N279S+G303R+E390A+G475K H1*+G7A+W140Y+R180*+S181*+T182D+A185Q+N194F+Y242F+S243Q+E259G+N279S+G303R+E390A+G475K H1*+G7A+W140Y+R180*+S181*+T182D+N194F+Y242F+S243Q+E259G+F266Y+N279S+G303R+E390A+G475K H1*+G7A+W140Y+R180*+S181*+T182D+E189P+N194F+Y242F+S243Q+E259G+N279S+K280Y+G303R+E390A+G475K H1*+G7A+W140Y+R180*+S181*+E189P+N194F+Y242F+E259G+F266Y+N279S+K280Y+G303R+E390A+G475K H1*+G7A+W140Y+R180*+S181*+A185Q+E189P+N194F+L205Y+Y242F+S243Q+E259G+N279S+K280Y+G303R+E390A+G475K H1*+G7A+W140Y+R180*+S181*+T182D+A185Q+N194F+Y242F+E259G+F266Y+N279S+G303R+E390A+G475K H1*+G7A+W140Y+R180*+S181*+A185Q+N194F+Y242F+E259G+F266Y+N279S+K280Y+G303R+E390A+G475K H1*+G7A+W140Y+R180*+S181*+A185Q+E189P+L205Y+Y242F+E259G+N279S+K280Y+G303R+E390A+G475K H1*+G7A+W140Y+R180*+S181*+Y242F+S243Q+E259G+F266Y+N279S+K280Y+G303R+E390A+G475K H1*+G7A+W140Y+R180*+S181*+T182D+E189P+L205Y+Y242F+S243Q+E259G+N279S+K280Y+G303R+E390A+I409L+G475K H1*+G7A+A41D+W140Y+R180*+S181*+N194F+L205Y+Y242F+E259G+N279S+G303R+E390A+G475K H1*+G7A+T40G+W140Y+R180*+S181*+N194F+L205Y+Y242F+E259G+N279S+G303R+E390A+G475K L63F+W140Y+L173F+R180*+S181*+N194F+L205Y+Y242F+E259G+N279S+H323W W140Y+L173F+R180*+S181*+N194F+L205Y+N225S+Y242F+E259G+N279S+G303R+H323A H1*+N3D+G7A+D118T+W140Y+R180*+S181*+T182S+E189P+N194F+L205Y+Y242F+E259G+N279S+K280Y+S302Y+E390A+N474L H1*+N3D+G7A+D118T+W140Y+R180*+S181*+T182S+E189P+N194F+L205Y+Y242F+E259G+N279S+K280Y+G303R+Y362V+E390A+H420A+N474L H1*+N3D+G7A+D118T+W140Y+R180*+S181*+T182S+E189P+N194F+L205Y+Y242F+E259G+N279S+K280Y+S302T+V365I+E390A+N474L H1*+G7A+D118T+W140Y+R180*+S181*+T182S+E189P+N194F+L205Y+Y242F+E259G+N279S+K280Y+S302Y+E390A+H420A+G475K H1*+G7A+D118T+W140Y+R180*+S181*+T182S+E189P+N194F+L205Y+Y242F+E259G+N279S+K280Y+S302Y+E390A+H420A+G475K H1*+N3D+G7A+D118T+W140Y+R180*+S181*+T182S+E189P+N194F+L205Y+Y242F+E259G+N279S+K280Y+S302Y+E390A+N474L H1*+N3D+G7A+D118T+W140Y+R180*+S181*+T182S+E189P+N194F+L205Y+Y242F+E259G+N279S+K280Y+S302T+E390A+H420A+G475K H1*+G7A+D118T+W140Y+R180*+S181*+T182S+E189P+N194F+L205Y+Y242F+E259G+N279S+K280Y+S302Y+E390A+H420A+G475K H1*+N3D+G7A+D118T+W140Y+R180*+S181*+T182S+E189P+N194F+L205Y+Y242F+E259G+N279S+K280Y+S302T+E390A+H420A+G475K H1*+G7A+D118T+W140Y+R180*+S181*+T182S+E189P+N194F+L205Y+Y242F+E259G+N279S+K280Y+G303R+E390A+H420A+G476A H1*+N3D+G7A+D118T+W140Y+R180*+S181*+T182S+E189P+N194F+L205Y+Y242F+E259G+N279S+K280Y+S302T+E390A+G475K H1*+N3G+G7A+D118T+W140Y+R180*+S181*+T182S+E189P+N194F+L205Y+Y242F+E259G+N279S+K280Y+S302T+E390A+H420A+G475K H1*+G7A+D118T+W140Y+R180*+S181*+T182S+E189P+N194F+L205Y+Y242F+E259G+N279S+K280Y+G303R+E390A+H420A+N474L H1*+N3D+G7A+R37A+D118T+W140Y+R180*+S181*+T182S+E189P+N194F+L205Y+Y242F+E259G+N279S+K280Y+G303R+N394D+N474F H1*+G7A+D118T+W140Y+R180*+S181*+T182S+E189P+N194F+L205Y+Y242F+E259G+N279S+K280Y+G303R+N394W+H420P+N474L H1*+G7A+G73L+D118T+W140Y+R180*+S181*+T182S+E189P+N194F+L205Y+Y242F+E259G+N279S+K280Y+S302Y+E390A+G475K H1*+G7A+D118T+W140Y+R180*+S181*+T182S+E189P+N194F+L205Y+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+S302Y+E390A+N474L H1*+G7A+D118T+W140Y+R180*+S181*+T182S+E189P+N194F+L205Y+Y242F+E259G+N279S+K280Y+S302T+E390A+N474L H1*+G7A+D118T+W140Y+R180*+S181*+T182S+E189P+N194F+L205Y+Y242F+E259G+N279S+K280Y+G303R+E390A+G476A H1*+N3D+G7A+D118T+W140Y+R180*+S181*+T182S+E189P+N194F+L205Y+Y242F+E259G+N279S+K280Y+S302Y+E390A+H420A+G475K H1*+N3D+G7A+D118T+W140Y+R180*+S181*+T182S+E189P+N194F+L205Y+Y242F+E259G+N279S+K280Y+S302Y+E390A+G475K H1*+G7A+D118T+W140Y+R180*+S181*+T182S+E189P+N194F+L205Y+Y242F+E259G+N279S+K280Y+S302Y+N394W+G475K H1*+N3D+G7A+K93R+D118T+W140Y+R180*+S181*+T182S+E189P+N194F+L205Y+Y242F+E259G+N279S+K280Y+G303R+E390A+H420A+N474L H1*+G7A+R37N+D118T+W140Y+R180*+S181*+T182S+E189P+N194F+L205Y+Y242F+E259G+N279S+K280Y+S302Y+E390A+G475K H1*+N3D+G7A+D118T+W140Y+R180*+S181*+T182S+E189P+N194F+L205Y+Y242F+E259G+N279S+K280Y+G303R+E390D+H420P+G475K H1*+G7A+D118T+W140Y+R180*+S181*+T182S+E189P+N194F+L205Y+Y242F+E259G+N279S+K280Y+S302T+N394W+G475K H1*+N3G+G7A+D118T+W140Y+R180*+S181*+T182S+E189P+N194F+L205Y+Y242F+E259G+N279S+K280Y+S302T+E390A+H420A+G475K H1*+N3D+G7A+D118T+W140Y+R180*+S181*+T182S+E189P+N194F+L205Y+Y242F+E259G+N279S+K280Y+G303R+N394W+H420A+G475K H1*+G7A+R37N+D118T+W140Y+R180*+S181*+T182S+E189P+N194F+L205Y+Y242F+E259G+N279S+K280Y+G303R+E390A+H420A+G475K H1*+N3D+G7A+D118T+W140Y+R180*+S181*+T182S+E189P+N194F+L205Y+Y242F+E259G+N279S+K280Y+S302T+E390A+G476H H1*+G7A+D118T+W140Y+R180*+S181*+T182S+E189P+N194F+L205Y+Y242F+E259G+N279S+K280Y+G303R+N394D+H420A+G475K H1*+N3D+G7A+D118T+W140Y+R180*+S181*+T182S+E189P+N194F+L205Y+Y242F+E259G+N279S+K280Y+G303R+E390A+H420A+G475K H1*+G7A+D118T+W140Y+R180*+S181*+T182S+E189P+N194F+L205Y+Y242F+E259G+N279S+K280Y+S302T+E390A+G476A H1*+G7A+D118T+W140Y+R180*+S181*+T182S+E189P+N194F+L205Y+Y242F+E259G+N279S+K280Y+G303R+E390A+H420P+G475K H1*+N3D+G7A+D118T+W140Y+R180*+S181*+T182S+E189P+N194F+L205Y+Y242F+E259G+N279S+K280Y+G303R+E390A+H420A+N474L N3G+G7A+D118T+W140Y+R180*+S181*+T182S+E189P+N194F+L205Y+Y242F+E259G+N279S+K280Y+G303R+E390A+H420A+N474L H1*+G7A+R37N+D118T+W140Y+R180*+S181*+T182S+E189P+N194F+L205Y+Y242F+E259G+N279S+K280Y+G303R+N394W+H420A+G475K H1*+N3D+G7A+R37N+D118T+Q138V+W140Y+R180*+S181*+T182S+E189P+N194F+L205Y+Y242F+E259G+N279S+K280Y+G303R+E390A+G475K H1*+G7A+D118T+W140Y+R180*+S181*+T182S+E189P+N194F+L205Y+Y242F+E259G+N279S+K280Y+S302Y+E390A+G476H H1*+G7A+D118T+Y135E+W140Y+R180*+S181*+T182S+E189P+N194F+L205Y+Y242F+E259G+N279S+K280Y+S302T+E390A+N474L H1*+G7A+D118T+W140Y+R180*+S181*+T182S+E189P+N194F+L205Y+Y242F+E259G+N279S+K280Y+G303R+N394W+H420A+G475K H1*+G7A+D118T+W140Y+R180*+S181*+T182S+E189P+N194F+L205Y+Y242F+E259G+N279S+K280Y+S302Y+E390A+G475K H1*+N3D+G7A+D118T+W140Y+R180*+S181*+T182S+E189P+N194F+L205Y+Y242F+E259G+N279S+K280Y+G303R+E390A+H420P+G475K H1*+G7A+V103I+D118T+W140Y+R180*+S181*+T182S+E189P+N194F+L205Y+Y242F+E259G+N279S+K280Y+S302Y+E390A+N474L H1*+G7A+D118T+W140Y+R180*+S181*+T182S+E189P+N194F+L205Y+Y242F+E259G+M260D+N279S+K280Y+S302Y+E390A+G475K H1*+G7A+D118T+W140Y+R180*+S181*+T182S+E189P+N194F+L205Y+Y242F+E259G+N279S+K280Y+S302Y+E390A+N474L H1*+G7A+D118T+W140Y+R180*+S181*+T182S+E189P+N194F+L205Y+Y242F+E259G+N279S+K280Y+S302T+E390A+G475K H1*+G7A+D118T+Q138V+W140Y+R180*+S181*+T182S+E189P+N194F+L205Y+Y242F+E259G+N279S+K280Y+G303R+H323W+E390A+N474L H1*+G7A+D118T+W140Y+R180*+S181*+T182S+E189P+N194F+L205Y+Y242F+E259G+N279S+K280Y+S302Y+E390A+G476H H1*+G7A+D118T+W140Y+R180*+S181*+T182S+E189P+N194F+L205Y+Y242F+E259G+N279S+K280Y+S302T+E390A+N474L H1*+G7A+D118T+W140Y+P146H+R180*+S181*+T182S+E189P+N194F+L205Y+Y242F+E259G+N279S+K280Y+S302T+E390A+G475K H1*+G7A+D118T+W140Y+R180*+S181*+T182S+E189P+N194F+L205Y+Y242F+E259G+F261P+N279S+K280Y+G303R+E390A+G475K H1*+G7A+T40G+D118T+W140Y+R180*+S181*+T182S+E189P+N194F+L205Y+Y242F+E259G+N279S+K280Y+S302Y+E390A+G475K H1*+G7A+Q98L+D118T+W140Y+R180*+S181*+T182S+E189P+N194F+L205Y+Y242F+E259G+N279S+K280Y+S302Y+E390A+G475K H1*+G7A+D118T+W140Y+R180*+S181*+T182S+E189P+N194F+L205Y+Y242F+E259G+N279S+K280Y+G303R+E390A+N474L H1*+G7A+D118T+W140Y+R180*+S181*+T182S+E189P+N194F+L205Y+Y242F+E259G+N279S+K280Y+G303R+P321H+E390A+G475K H1*+G7A+D118T+W140Y+R180*+S181*+T182S+E189P+N194F+L205Y+Y242F+E259G+N279S+K280Y+G303R+P321H+E390A+N474L H1*+G7A+D118T+S132D+W140Y+R180*+S181*+T182S+E189P+N194F+L205Y+Y242F+E259G+N279S+K280Y+S302Y+E390A+G475K H1*+G7A+D118T+W140Y+R180*+S181*+T182S+E189P+N194F+L205Y+Y242F+E259G+N279S+K280Y+G303R+N394D+R414M+G475K H1*+G7A+T40G+D118T+W140Y+R180*+S181*+T182S+E189P+N194F+L205Y+Y242F+E259G+N279S+K280Y+G303R+E390A+G476A H1*+G7A+D118T+W140Y+R180*+S181*+T182S+E189P+N194F+L205Y+T224F+Y242F+E259G+N279S+K280Y+G303R+N394W+H420A+G475K H1*+G7A+D118T+W140Y+R180*+S181*+T182S+E189P+N194F+L205Y+Y242F+E259G+M260D+N279S+K280Y+S302T+E390A+G475K H1*+G7A+D118T+W140Y+R180*+S181*+T182S+E189P+N194F+L205Y+Y242F+E259G+M260W+N279S+K280Y+S302Y+E390A+G476A H1*+G7A+D118T+Y135E+W140Y+R180*+S181*+T182S+E189P+N194F+L205Y+Y242F+E259G+M260D+N279S+K280Y+G303R+W346N+E390A+G475K H1*+G7A+D118T+W140Y+R180*+S181*+T182S+E189P+N194F+L205Y+Y242F+E259G+N279S+K280Y+G303R+P321H+Q360Y+E390A+G476A H1*+G7A+D118T+W140Y+R180*+S181*+T182S+E189P+N194F+L205Y+Y242F+E259G+M260D+N279S+K280Y+S302Y+E390A+N470M+G475K H1*+G7A+D118T+W140Y+R180*+S181*+T182S+E189P+N194F+L205Y+Y242F+E259G+M260W+N279S+K280Y+G303R+E390A+G476A H1*+G7A+D118T+W140Y+R180*+S181*+T182S+E189P+N194F+L205Y+T224Y+Y242F+N279S+K280Y+G303R+E390A+G476A H1*+G7A+D118T+W140Y+R180*+S181*+T182S+E189P+N194F+L205Y+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+S302Y+E390A+N474L H1*+G7A+D118T+W140Y+R180*+S181*+T182S+E189P+N194F+L205Y+Y242F+E259G+N279S+K280Y+S302Y+E390A+G476H H1*+G7A+D118T+W140Y+R180*+S181*+T182S+E189P+N194F+L205Y+Y242F+E259G+N279S+K280Y+S302Y+E390A+N474F H1*+G7A+D118T+W140Y+R180*+S181*+T182S+E189P+N194F+L205Y+Y242F+E259G+M260W+N279S+K280Y+G303R+E390A+G476A H1*+G7A+D118T+W140Y+R180*+S181*+T182S+E189P+N194F+L205Y+Y242F+E259G+M260W+N279S+K280Y+S302Y+E390A+G475K H1*+N3D+G7A+D118T+W140Y+R180*+S181*+T182S+E189P+N194F+L205Y+Y242F+E259G+N279S+K280Y+S302T+E390A+H420A+G475K H1*+G7A+D118T+W140Y+R180*+S181*+T182S+E189P+N194F+L205Y+T224Y+Y242F+E259G+N279S+K280Y+G303R+E390A+N474L H1*+G7A+D118T+W140Y+R180*+S181*+T182S+E189P+N194F+L205Y+Y242F+E259G+N279S+K280Y+S302Y+E390A+G475K H1*+G7A+D118T+W140Y+R180*+S181*+T182S+E189P+N194F+L205Y+Y242F+E259G+N279S+K280Y+S302Y+E390A+H420A+G475K H1*+G7A+D118T+W140Y+R180*+S181*+T182S+E189P+N194F+L205Y+Y242F+E259G+N279S+K280Y+G303R+E390A+G476A H1*+G7A+D118T+W140Y+R180*+S181*+T182S+E189P+N194F+L205Y+Y242F+E259G+N279S+K280Y+S302T+E390A+N474L H1*+G7A+D118T+W140Y+P146H+R180*+S181*+T182S+E189P+N194F+L205Y+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+S302Y+E390A+G475K H1*+N3G+G7A+D118T+W140Y+R180*+S181*+T182S+E189P+N194F+L205Y+Y242F+E259G+N279S+K280Y+S302T+E390A+G475K H1*+G7A+D118T+S132L+W140Y+R180*+S181*+T182S+E189P+N194F+L205Y+Y242F+E259G+M260D+N279S+K280Y+G303R+E390A+G475K H1*+G7A+D118T+W140Y+R180*+S181*+T182S+E189P+N194F+L205Y+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+G303R+E390A+G475K H1*+D118T+W140Y+R180*+S181*+T182S+E189P+N194F+L205Y+Y242F+E259G+N279S+K280Y+G303R+E390A+G475K H1*+G7A+W140Y+R180*+S181*+T182S+E189P+N194F+L205Y+Y242F+E259G+N279S+K280Y+G303R+E390A+G475K H1*+G7A+D118T+R180*+S181*+T182S+E189P+N194F+L205Y+Y242F+E259G+N279S+K280Y+G303R+E390A+G475K H1*+G7A+D118T+W140Y+R180*+S181*+E189P+N194F+L205Y+Y242F+E259G+N279S+K280Y+G303R+E390A+G475K H1*+G7A+D118T+W140Y+R180*+S181*+T182S+N194F+L205Y+Y242F+E259G+N279S+K280Y+G303R+E390A+G475K H1*+G7A+D118T+W140Y+R180*+S181*+T182S+E189P+L205Y+Y242F+E259G+N279S+K280Y+G303R+E390A+G475K H1*+G7A+D118T+W140Y+R180*+S181*+T182S+E189P+N194F+Y242F+E259G+N279S+K280Y+G303R+E390A+G475K H1*+G7A+D118T+W140Y+R180*+S181*+T182S+E189P+N194F+L205Y+E259G+N279S+K280Y+G303R+E390A+G475K H1*+G7A+D118T+W140Y+R180*+S181*+T182S+E189P+N194F+L205Y+Y242F+N279S+K280Y+G303R+E390A+G475K H1*+G7A+D118T+W140Y+R180*+S181*+T182S+E189P+N194F+L205Y+Y242F+E259G+K280Y+G303R+E390A+G475K H1*+G7A+D118T+W140Y+R180*+S181*+T182S+E189P+N194F+L205Y+Y242F+E259G+N279S+G303R+E390A+G475K H1*+G7A+D118T+W140Y+R180*+S181*+T182S+E189P+N194F+L205Y+Y242F+E259G+N279S+K280Y+E390A+G475K H1*+G7A+D118T+W140Y+R180*+S181*+T182S+E189P+N194F+L205Y+Y242F+E259G+N279S+K280Y+G303R+G475K H1*+G7A+D118T+W140Y+R180*+S181*+T182S+E189P+N194F+L205Y+Y242F+E259G+N279S+K280Y+G303R+E390A H1*+G7A+D118T+W140Y+R180*+S181*+T182S+E189P+N194F+L205V+Y242F+E259G+N279S+K280Y+G303R+E390A+G475K H1*+G7A+D118T+W140Y+R180*+S181*+T182S+E189P+N194F+L205N+Y242F+E259G+N279S+K280Y+G303R+E390A+G475K H1*+G7A+D118T+W140Y+R180*+S181*+T182S+E189P+N194F+L205Q+Y242F+E259G+N279S+K280Y+G303R+E390A+G475K H1*+G7A+D118T+W140Y+R180*+S181*+T182S+A185Q+E189P+N194F+L205Y+Y242F+E259G+N279S+K280Y+G303R+E390A+G475K H1*+G7A+D118T+W140Y+R180*+S181*+T182S+E189P+N194F+L205Y+Y242F+E259G+F266Y+N279S+K280Y+G303R+E390A+G475K H1*+G7A+D118T+W140Y+R180*+S181*+A185Q+E189P+N194F+L205Y+Y242F+E259G+N279S+K280Y+G303R+E390A+G475K H1*+G7A+D118T+W140Y+R180*+S181*+T182S+A185Q+E189P+N194F+L205Y+Y242F+E259G+F266Y+N279S+K280Y+G303R+E390A+G475K H1*+G7A+D118T+W140Y+R180*+S181*+T182S+A185Q+E189P+N194F+L205N+Y242F+E259G+F266Y+N279S+K280Y+G303R+E390A+G475K H1*+G7A+D118T+W140Y+R180*+S181*+T182S+A185D+E189P+N194F+Y242F+E259G+F266Y+N279S+K280Y+G303R+E390A+G475K H1*+G7A+D118T+W140Y+R180*+S181*+T182S+A185D+E189P+Y242F+S243Q+E259G+F266Y+N279S+K280Y+G303R+E390A+G475K H1*+G7A+D118T+W140Y+R180Q+S181*+T182*+A185Q+E189P+Y242F+S243Q+E259G+F266Y+N279S+K280Y+G303R+E390A+G475K H1*+G7A+D118T+W140Y+R180*+S181*+T182S+A185Q+E189P+Y242F+E259G+F266Y+N279S+K280Y+G303R+E390A+G475K H1*+G7A+D118T+W140Y+R180*+S181*+T182S+A185Q+E189P+L205Y+Y242F+E259G+F266Y+N279S+K280Y+G303R+E390A+G475K H1*+G7A+D118T+W140Y+R180*+S181*+T182S+A185D+E189P+L205Y+Y242F+E259G+F266Y+N279S+K280Y+G303R+E390A+G475K H1*+G7A+D118T+W140Y+R180Q+S181*+T182*+A185Q+E189P+N194F+L205Y+Y242F+E259G+F266Y+N279S+K280Y+G303R+E390A+G475K H1*+N3D+G7A+D118T+W140Y+R180*+S181*+T182S+E189P+N194F+L205Y+Y242F+E259G+N279S+K280Y+S302T+E390A+H420A+G475K H1*+G7A+T40G+D118T+W140Y+R180*+S181*+T182S+E189P+N194F+L205Y+Y242F+E259G+N279S+K280Y+S302Y+E390A+G475K H1*+G7A+T40G+D118T+W140Y+R180*+S181*+T182S+E189P+N194F+L205Y+Y242F+E259G+N279S+K280Y+S302Y+E390A+G475K H1*+G7A+T40G+D118T+W140Y+R180*+S181*+T182S+E189P+N194F+L205Y+Y242F+E259G+N279S+K280Y+G303R+E390A+G476A H1*+G7A+T40G+D118T+W140Y+R180*+S181*+T182S+E189P+N194F+L205Y+Y242F+E259G+N279S+K280Y+G303R+E390A+G476A H1*+G7A+D118T+W140Y+R180*+S181*+T182S+E189P+N194F+L205Y+Y242F+E259G+N279S+K280Y+G303R+N394D+N422V+G475K H1*+G7A+Q98L+D118T+W140Y+R180*+S181*+T182S+E189P+N194F+L205Y+Y242F+E259G+N279S+K280Y+G303R+N394D+H420A+G475K H1*+G7A+D118T+W140Y+R180*+S181*+T182S+E189P+N194F+L205Y+Y242F+E259G+M260D+N279S+K280Y+G303R+Y362V+E390A+G475K H1*+G7A+D118T+Y135E+W140Y+R180*+S181*+T182S+E189P+N194F+L205Y+Y242F+E259G+F261P+N279S+K280Y+G303R+Y362V+E390A+G475K H1*+G7A+D118T+W140Y+R180*+S181*+T182S+E189P+N194F+L205Y+Y242F+E259G+M260D+N279S+K280Y+G303R+E390A+G475K H1*+G7A+D118T+W140Y+R180*+S181*+T182S+E189P+N194F+L205Y+Y242F+T256D+N279S+K280Y+G303R+E390A+G475K H1*+G7A+D118T+Y135E+W140Y+R180*+S181*+T182S+E189P+N194F+L205Y+T224F+Y242F+E259G+N279S+K280Y+G303R+E390A+G475K H1*+G7A+D118T+W140Y+R180*+S181*+T182S+E189P+N194F+L205Y+Y242F+T256D+N279S+K280Y+G303R+E390A+G475K H1*+G7A+D118T+W140Y+R180*+S181*+T182S+E189P+N194F+L205Y+Y242F+T256D+N279S+K280Y+G303R+E390A+G475K H1*+G7A+D118T+W140Y+R180*+S181*+T182S+E189P+N194F+L205Y+Y242F+E259G+F261P+N279S+K280Y+G303R+E390A+G475K H1*+G7A+D118T+W140Y+R180*+S181*+T182S+E189P+N194F+L205Y+Y242F+T256D+N279S+K280Y+G303R+E390A+G475K H1*+G7A+D118T+W140Y+R180*+S181*+T182S+E189P+N194F+L205Y+Y242F+E259G+M260D+N279S+K280Y+G303R+Y371N+E390A+G475K H1*+G7A+D118T+W140Y+R180*+S181*+T182S+E189P+N194F+L205Y+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+S302T+E390A+N474F H1*+G7A+D118T+Y135E+W140Y+R180*+S181*+T182S+E189P+N194F+L205Y+Y242F+E259G+N279S+K280Y+G303R+P321Y+E390A+G475K H1*+G7A+D118T+S132D+W140Y+R180*+S181*+T182S+E189P+N194F+L205Y+Y242F+E259G+N279S+K280Y+G303R+P321Y+E390A+G475K H1*+N3G+G7A+D118T+Q138V+W140Y+R180*+S181*+T182S+E189P+N194F+L205Y+Y242F+E259G+N279S+K280Y+G303R+E390A+H420A+G476A H1*+G7A+D118T+W140Y+R180*+S181*+T182S+E189P+N194F+L205Y+Y242F+E259G+N279S+K280Y+S302Y+E390A+G475K H1*+N3D+G7A+D118T+W140Y+R180*+S181*+T182S+E189P+N194F+L205Y+Y242F+E259G+N279S+K280Y+S302T+E390A+G475K H1*+G7A+D118T+Q138V+W140Y+R180*+S181*+T182S+E189P+N194F+L205Y+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+S302T+E390A+G475K H1*+G7A+D118T+W140Y+R180*+S181*+T182S+E189P+N194F+L205Y+Y242F+T256D+N279S+K280Y+G303R+E390A+N470M+G475K H1*+N3D+G7A+D118T+W140Y+R180*+S181*+T182S+E189P+N194F+L205Y+Y242F+E259G+N279S+K280Y+S302Y+E390A+G475K H1*+N3D+G7A+D118T+W140Y+R180*+S181*+T182S+E189P+N194F+L205Y+Y242F+E259G+N279S+K280Y+S302T+E390A+G475K H1*+G7A+D118T+S132D+W140Y+R180*+S181*+T182S+E189P+N194F+L205Y+Y242F+E259G+N279S+K280Y+G303R+Y362V+E390A+N474L H1*+G7A+D118T+W140Y+R180*+S181*+T182S+E189P+N194F+L205Y+Y242F+E259G+N279S+K280Y+S302T+E390A+G475K H1*+G7A+D118T+Q138V+W140Y+R180*+S181*+T182S+E189P+N194F+L205Y+Y242F+E259G+N279S+K280Y+G303R+E390A+G476A H1*+G7A+D118T+W140Y+R180*+S181*+T182S+E189P+N194F+L205Y+Y242F+E259G+N279S+K280Y+S302T+Y362V+E390A+G475K H1*+G7A+D118T+W140Y+R180*+S181*+T182S+E189P+N194F+L205Y+Y242F+E259G+N279S+K280Y+G303R+Y362V+E390A+G476A H1*+G7A+D118T+W140Y+R180*+S181*+T182S+E189P+N194F+L205Y+Y242F+E259G+M260D+N279S+K280Y+G303R+E390A+N474L H1*+G7A+D118T+W140Y+R180*+S181*+T182S+E189P+N194F+L205Y+T224Y+Y242F+E259G+N279S+K280Y+S302Y+E390A+G475K H1*+G7A+D118T+W140Y+R180*+S181*+T182S+E189P+N194F+L205Y+Y242F+E259G+N279S+K280Y+S302Y+Y362V+E390A+G475K H1*+G7A+D118T+W140Y+R180*+S181*+T182S+E189P+N194F+L205Y+Y242F+E259G+M260W+N279S+K280Y+S302T+E390A+G475K H1*+G7A+D118T+W140Y+R180*+S181*+T182S+E189P+N194F+L205Y+T224Y+Y242F+E259G+N279S+K280Y+G303R+E390A+G476A H1*+G7A+D118T+W140Y+R180*+S181*+T182S+E189P+N194F+L205Y+Y242F+E259G+M260D+N279S+K280Y+G303R+E390A+N474F H1*+G7A+D118T+W140Y+R180*+S181*+T182S+E189P+N194F+L205Y+Y242F+E259G+N279S+K280Y+S302Y+E390A+N474L H1*+G7A+D118T+W140Y+R180*+S181*+T182S+E189P+N194F+L205Y+Y242F+E259G+N279S+K280Y+S302Y+E390A+G476H H1*+G7A+D118T+W140Y+R180*+S181*+T182S+E189P+N194F+L205Y+Y242F+E259G+M260D+N279S+K280Y+G303R+E390A+H420A+G475K H1*+G7A+D118T+Y135E+W140Y+R180*+S181*+T182S+E189P+N194F+L205Y+Y242F+E259G+N279S+K280Y+G303R+N394W+H420A+G475K H1*+N3D+G7A+D118T+W140Y+R180*+S181*+T182S+E189P+N194F+L205Y+Y242F+E259G+N279S+K280Y+G303R+N394D+H420A+G475K H1*+G7A+D118T+Q138V+W140Y+R180*+S181*+T182S+E189P+N194F+L205Y+Y242F+E259G+N279S+K280Y+G303R+E390A+H420Q+G475K H1*+G7A+R37N+D118T+W140Y+R180*+S181*+T182S+E189P+N194F+L205Y+Y242F+E259G+N279S+K280Y+G303R+E390A+H420P+G476H H1*+G7A+R37A+Q98L+D118T+W140Y+R180*+S181*+T182S+E189P+N194F+L205Y+Y242F+E259G+N279S+K280Y+S302Y+E390A+G475K H1*+G7A+R37A+D118T+W140Y+R180*+S181*+T182S+E189P+N194F+L205Y+Y242F+E259G+N279S+K280Y+G303R+E390A+H420A+G475K H1*+G7A+D118T+W140Y+R180*+S181*+T182S+E189P+N194F+L205Y+Y242F+E259G+N279S+K280Y+G303R+E390A+H420P+G475K H1*+G7A+D118T+W140Y+R180*+S181*+T182S+E189P+N194F+L205Y+Y242F+E259G+N279S+K280Y+G303R+E390A+N470M+G476A H1*+G7A+D118T+W140Y+R180*+S181*+T182S+E189P+N194F+L205Y+Y242F+E259G+M260D+N279S+K280Y+G303R+E390A+H420P+G476A H1*+G7A+D118T+W140Y+R180*+S181*+T182S+E189P+N194F+L205Y+Y242F+E259G+N279S+K280Y+S302T+E390A+H420A+G475K H1*+G7A+R37A+D118T+W140Y+R180*+S181*+T182S+E189P+N194F+L205Y+Y242F+E259G+N279S+K280Y+S302Y+E390A+H420A+G475K H1*+G7A+D118T+Y135E+W140Y+R180*+S181*+T182S+E189P+N194F+L205Y+Y242F+E259G+N279S+K280Y+S302T+Y371N+E390A+G475K H1*+G7A+R37N+D118T+S132D+W140Y+R180*+S181*+T182S+E189P+N194F+L205Y+Y242F+E259G+N279S+K280Y+G303R+E390A+N470M+G475K H1*+G7A+D118T+W140Y+R180*+S181*+T182S+E189P+N194F+L205Y+Y242F+E259G+N279S+K280Y+G303R+E390A+H420A+G475K H1*+G7A+D118T+W140Y+R180*+S181*+T182S+E189P+N194F+L205Y+Y242F+E259G+N279S+K280Y+G303R+H323W+E390A+H420A+N474L H1*+G7A+D118T+W140Y+R180*+S181*+T182S+E189P+N194F+L205Y+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+G303R+N394W+H420P+G475K H1*+G7A+D118T+W140Y+R180*+S181*+T182S+E189P+N194F+L205Y+Y242F+E259G+N279S+K280Y+S302T+H323W+E390A+G475K H1*+G7A+D118T+W140Y+R180*+S181*+T182S+E189P+N194F+L205Y+Y242F+E259G+N279S+K280Y+S302T+E390A+G475K H1*+G7A+D118T+Y135E+W140Y+R180*+S181*+T182S+E189P+N194F+L205Y+Y242F+E259G+N279S+K280Y+G303R+E390A+R414M+G476H H1*+G7A+R37N+D118T+W140Y+R180*+S181*+T182S+E189P+N194F+L205Y+Y242F+E259G+N279S+K280Y+S302Y+E390A+H420A+G475K H1*+G7A+R37A+D118T+W140Y+R180*+S181*+T182S+E189P+N194F+L205Y+Y242F+E259G+N279S+K280Y+S302T+E390A+H420A+G476A H1*+G7A+R37N+D118T+W140Y+R180*+S181*+T182S+E189P+N194F+L205Y+Y242F+E259G+N279S+K280Y+S302Y+E390A+H420A+G475K H1*+G7A+R37A+D118T+W140Y+R180*+S181*+T182S+E189P+N194F+L205Y+Y242F+E259G+N279S+K280Y+S302Y+E390A+N474L H1*+G7A+R37A+D118T+W140Y+R180*+S181*+T182S+E189P+N194F+L205Y+Y242F+E259G+N279S+K280Y+S302Y+E390A+G475K H1*+N3D+G7A+R37A+D118T+W140Y+R180*+S181*+T182S+E189P+N194F+L205Y+Y242F+E259G+N279S+K280Y+S302Y+N394D+H420A+G475K H1*+G7A+R37A+D118T+W140Y+R180*+S181*+T182S+E189P+N194F+L205Y+Y242F+E259G+N279S+K280Y+S302Y+E390A+H420A+G476H H1*+G7A+R37A+D118T+W140Y+R180*+S181*+T182S+E189P+N194F+L205Y+Y242F+E259G+N279S+K280Y+S302Y+N394D+H420A+G475K H1*+G7A+R37A+D118T+W140Y+R180*+S181*+T182S+E189P+N194F+L205Y+Y242F+E259G+N279S+K280Y+S302Y+E390A+N474L H1*+G7A+R37N+D118T+W140Y+R180*+S181*+T182S+E189P+N194F+L205Y+Y242F+E259G+N279S+K280Y+S302T+E390A+H420A+G475K H1*+G7A+R37A+D118T+W140Y+R180*+S181*+T182S+E189P+N194F+L205Y+Y242F+E259G+N279S+K280Y+S302Y+E390A+H420A+G475K H1*+G7A+R37N+D118T+W140Y+R180*+S181*+T182S+E189P+N194F+L205Y+Y242F+E259G+N279S+K280Y+S302T+E390A+H420A+G475K H1*+G7A+R37A+D118T+W140Y+R180*+S181*+T182S+E189P+N194F+L205Y+Y242F+E259G+N279S+K280Y+S302T+E390A+H420A+G475K H1*+G7A+D118T+W140Y+R180*+S181*+T182S+E189P+N194F+L205Y+Y242F+E259G+N279S+K280Y+S302T+N394W+H420A+G475K H1*+G7A+R37A+D118T+W140Y+R180*+S181*+T182S+E189P+N194F+L205Y+Y242F+E259G+N279S+K280Y+S302Y+N394D+G475K H1*+G7A+R37N+D118T+W140Y+R180*+S181*+T182S+E189P+N194F+L205Y+Y242F+E259G+N279S+K280Y+S302Y+E390A+H420A+G476H H1*+G7A+R37N+D118T+W140Y+R180*+S181*+T182S+E189P+N194F+L205Y+Y242F+E259G+N279S+K280Y+S302T+N394D+G475K H1*+G7A+R37A+D118T+W140Y+R180*+S181*+T182S+E189P+N194F+L205Y+Y242F+E259G+N279S+K280Y+S302T+E390A+G475K H1*+G7A+R37N+D118T+W140Y+R180*+S181*+T182S+E189P+N194F+L205Y+Y242F+E259G+N279S+K280Y+S302T+N394D+H420A+G475K H1*+N3G+G7A+D118T+W140Y+R180*+S181*+T182S+E189P+N194F+L205Y+Y242F+E259G+N279S+K280Y+S302T+E390A+H420A+G475K H1*+N3D+G7A+D118T+W140Y+R180*+S181*+T182S+E189P+N194F+L205Y+Y242F+E259G+N279S+K280Y+S302T+E390A+G475K H1*+N3G+G7A+D118T+W140Y+R180*+S181*+T182S+E189P+N194F+L205Y+Y242F+E259G+N279S+K280Y+S302T+E390A+H420A+G475K H1*+N3D+G7A+D118T+W140Y+R180*+S181*+T182S+E189P+N194F+L205Y+Y242F+E259G+N279S+K280Y+S302T+E390A+G475K H1*+N3D+G7A+D118T+W140Y+R180*+S181*+T182S+E189P+N194F+L205Y+Y242F+E259G+N279S+K280Y+S302T+E390A+G475K H1*+N3D+G7A+D118T+W140Y+R180*+S181*+T182S+E189P+N194F+L205Y+Y242F+E259G+N279S+K280Y+S302Y+E390A+N474L H1*+N3D+G7A+D118T+W140Y+R180*+S181*+T182S+E189P+N194F+L205Y+Y242F+E259G+N279S+K280Y+S302T+E390A+G475K H1*+N3D+G7A+D118T+W140Y+R180*+S181*+T182S+E189P+N194F+L205Y+Y242F+E259G+N279S+K280Y+S302Y+E390A+G475K H1*+N3D+G7A+D118T+W140Y+R180*+S181*+T182S+E189P+N194F+L205Y+Y242F+E259G+N279S+K280Y+S302Y+E390A+G475K H1*+N3D+G7A+R37N+D118T+W140Y+R180*+S181*+T182S+E189P+N194F+L205Y+Y242F+E259G+N279S+K280Y+G303R+E390A+H420A+G475K H1*+N3D+G7A+R37N+D118T+W140Y+R180*+S181*+T182S+E189P+N194F+L205Y+Y242F+E259G+N279S+K280Y+G303R+E390A+H420A+G475K H1*+N3D+G7A+R37A+D118T+W140Y+R180*+S181*+T182S+E189P+N194F+L205Y+Y242F+E259G+N279S+K280Y+G303R+E390A+G475K H1*+N3G+G7A+R37N+D118T+W140Y+R180*+S181*+T182S+E189P+N194F+L205Y+Y242F+E259G+N279S+K280Y+G303R+N394W+H420A+G475K H1*+N3D+G7A+R37N+D118T+W140Y+R180*+S181*+T182S+E189P+N194F+L205Y+Y242F+E259G+N279S+K280Y+G303R+E390A+H420A+G475K H1*+N3D+G7A+R37N+D118T+W140Y+R180*+S181*+T182S+E189P+N194F+L205Y+Y242F+E259G+N279S+K280Y+G303R+G361M+H420A+G475K H1*+N3D+G7A+R37A+D118T+W140Y+R180*+S181*+T182S+E189P+N194F+L205Y+Y242F+E259G+N279S+K280Y+G303R+E390A+H420A+G475K H1*+G7A+R37A+D118T+W140Y+R180*+S181*+T182S+E189P+N194F+L205Y+Y242F+E259G+M260D+N279S+K280Y+G303R+N394W+G475K H1*+G7A+R37N+D118T+W140Y+R180*+S181*+T182S+E189P+N194F+L205Y+Y242F+E259G+M260D+N279S+K280Y+G303R+E390A+G475K H1*+G7A+R37A+D118T+W140Y+R180*+S181*+T182S+E189P+N194F+L205Y+T224F+Y242F+N279S+K280Y+G303R+E390A+G475K H1*+G7A+R37N+D118T+W140Y+R180*+S181*+T182S+E189P+N194F+L205Y+Y242F+E259G+M260D+N279S+K280Y+G303R+N394D+G475K H1*+G7A+R37A+D118T+W140Y+R180*+S181*+T182S+E189P+N194F+L205Y+Y242F+E259G+M260D+N279S+K280Y+G303R+N394D+G475K H1*+G7A+R37N+D118T+W140Y+R180*+S181*+T182S+E189P+N194F+L205Y+Y242F+E259G+M260D+N279S+K280Y+G303R+N394D+G475K H1*+G7A+D118T+W140Y+R180*+S181*+T182S+E189P+N194F+L205Y+Y242F+E259G+M260D+N279S+K280Y+G303R+H420A+G475K H1*+G7A+R37A+D118T+W140Y+R180*+S181*+T182S+E189P+N194F+L205Y+Y242F+E259G+M260D+N279S+K280Y+G303R+E390A+G475K H1*+G7A+R37N+D118T+W140Y+R180*+S181*+T182S+E189P+N194F+L205Y+T224Y+Y242F+N279S+K280Y+G303R+E390A+G475K H1*+G7A+D118T+W140Y+R180*+S181*+T182S+E189P+N194F+L205Y+Y242F+E259G+N279S+K280Y+G303R+N394W+H420A+G475K H1*+G7A+D118T+W140Y+R180*+S181*+T182S+E189P+N194F+L205Y+Y242F+E259G+N279S+K280Y+G303R+N394W+H420A+N474L H1*+G7A+D118T+W140Y+P146H+R180*+S181*+T182S+E189P+N194F+L205Y+Y242F+E259G+N279S+K280Y+S302T+E390A+G476A H1*+G7A+D118T+W140Y+P146H+R180*+S181*+T182S+E189P+N194F+L205Y+Y242F+E259G+N279S+K280Y+S302Y+E390A+N474L H1*+G7A+D118T+W140Y+R180*+S181*+T182S+A185Q+E189P+N194F+Y242F+E259G+F266Y+N279S+K280Y+G303R+E390A+G475K H1*+G7A+D118T+W140Y+R180*+S181*+T182S+A185Q+E189P+N194F+L205Q+Y242F+E259G+F266Y+N279S+K280Y+G303R+E390A+G475K H1*+G7A+D118T+W140Y+R180Q+S181*+T182*+A185Q+E189P+N194F+Y242F+E259G+F266Y+N279S+K280Y+G303R+E390A+G475K H1*+G7A+D118T+W140Y+R180*+S181*+T182S+A185Q+E189P+Y242F+S243Q+E259G+F266Y+N279S+K280Y+G303R+E390A+G475K H1*+N3G+G7A+V104M+D118T+W140Y+R180*+S181*+T182S+E189P+N194F+L205Y+Y242F+E259G+N279S+K280Y+S302Y+N394W+G475K H1*+N3D+G7A+D118T+W140Y+R180*+S181*+T182S+E189P+N194F+L205Y+Y242F+E259G+N279S+K280Y+G303R+E390A+G475K H1*+N3G+G7A+D118T+W140Y+R180*+S181*+T182S+E189P+N194F+L205Y+Y242F+E259G+N279S+K280Y+G303R+N394W+H420A+G475K H1*+N3D+G7A+D118T+W140Y+R180*+S181*+T182S+E189P+N194F+L205Y+Y242F+E259G+N279S+K280Y+G303R+E390A+H420A+G475K H1*+N3D+G7A+D118T+S132D+W140Y+R180*+S181*+T182S+E189P+N194F+L205Y+Y242F+E259G+N279S+K280Y+G303R+E390A+H420A+G475K H1*+G7A+D118T+W140Y+R180*+S181*+T182S+E189P+N194F+L205Y+Y242F+E259G+N279S+K280Y+G303R+E390A+N470M+G475K H1*+G7A+D118T+W140Y+R180*+S181*+T182S+E189P+N194F+L205Y+Y242F+E259G+N279S+K280Y+S302Y+Y371N+E390A+G475K H1*+G7A+D118T+W140Y+R180*+S181*+T182S+E189P+N194F+L205Y+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+S302Y+E390A+G475K H1*+G7A+D118T+Y135E+W140Y+R180*+S181*+T182S+E189P+N194F+L205Y+Y242F+E259G+N279S+K280Y+S302Y+E390A+G475K H1*+G7A+D118T+W140Y+R180*+S181*+T182S+E189P+N194F+L205Y+T224F+Y242F+E259G+N279S+K280Y+S302T+E390A+G475K H1*+G7A+R37A+D118T+W140Y+R180*+S181*+T182S+E189P+N194F+L205Y+Y242F+E259G+M260D+N279S+K280Y+G303R+E390A+G475K H1*+G7A+R37N+D118T+W140Y+R180*+S181*+T182S+E189P+N194F+L205Y+Y242F+E259G+M260D+N279S+K280Y+G303R+E390A+G475K H1*+G7A+D118T+W140Y+R180*+S181*+T182S+E189P+N194F+L205Y+Y242F+E259G+M260D+N279S+K280Y+G303R+N394W+G475K H1*+G7A+D118T+W140Y+R180*+S181*+T182S+E189P+N194F+L205Y+Y242F+E259G+M260D+N279S+K280Y+G303R+E390A+G475K H1*+G7A+R37A+D118T+W140Y+R180*+S181*+T182S+E189P+N194F+L205Y+Y242F+E259G+M260W+N279S+K280Y+G303R+E390A+N470M+G475K H1*+G7A+R37N+D118T+W140Y+R180*+S181*+T182S+E189P+N194F+L205Y+Y242F+E259G+M260D+N279S+K280Y+G303R+E390A+G475K H1*+G7A+D118T+W140Y+R180*+S181*+T182S+E189P+N194F+L205Y+Y242F+E259G+M260D+N279S+K280Y+G303R+E390A+G475K H1*+G7A+D118T+W140Y+R180*+S181*+T182S+E189P+N194F+L205Y+Y242F+E259G+M260D+N279S+K280Y+G303R+N394D+G475K H1*+G7A+R37N+D118T+W140Y+R180*+S181*+T182S+E189P+N194F+L205Y+Y242F+E259G+M260W+N279S+K280Y+G303R+E390A+H420A+G475K H1*+G7A+D118T+W140Y+R180*+S181*+T182S+E189P+N194F+L205Y+Y242F+E259G+M260D+N279S+K280Y+G303R+E390A+N470M+G475K H1*+G7A+R37A+D118T+W140Y+R180*+S181*+T182S+E189P+N194F+L205Y+Y242F+E259G+M260D+N279S+K280Y+G303R+N394W+G475K H1*+G7A+R37A+D118T+W140Y+R180*+S181*+T182S+E189P+N194F+L205Y+Y242F+E259G+N279S+K280Y+G303R+E390A+H420A+G475K H1*+G7A+R37A+D118T+W140Y+R180*+S181*+T182S+E189P+N194F+L205Y+Y242F+E259G+M260D+N279S+K280Y+G303R+E390A+G475K H1*+G7A+R37A+D118T+W140Y+R180*+S181*+T182S+E189P+N194F+L205Y+T224Y+Y242F+E259G+N279S+K280Y+G303R+N394W+G475K H1*+G7A+R37N+D118T+W140Y+R180*+S181*+T182S+E189P+N194F+L205Y+Y242F+E259G+M260D+N279S+K280Y+G303R+N394W+H420A+G475K H1*+G7A+R37A+D118T+W140Y+R180*+S181*+T182S+E189P+N194F+L205Y+Y242F+E259G+M260D+N279S+K280Y+G303R+E390A+N470M+G475K H1*+G7A+R37A+D118T+W140Y+R180*+S181*+T182S+E189P+N194F+L205Y+Y242F+T256D+N279S+K280Y+G303R+E390A+G475K H1*+G7A+R37A+D118T+W140Y+R180*+S181*+T182S+E189P+N194F+L205Y+Y242F+E259G+F261P+N279S+K280Y+G303R+E390A+G475K H1*+G7A+R37A+D118T+W140Y+R180*+S181*+T182S+E189P+N194F+L205Y+Y242F+E259G+M260D+N279S+K280Y+G303R+N394D+H420A+G475K H1*+G7A+R37A+D118T+W140Y+R180*+S181*+T182S+E189P+N194F+L205Y+Y242F+E259G+M260D+N279S+K280Y+G303R+N394D+G475K H1*+G7A+R37N+D118T+W140Y+N174K+R180*+S181*+T182S+E189P+N194F+L205Y+Y242F+E259G+M260D+N279S+K280Y+G303R+N394W+G475K H1*+G7A+R37A+D118T+W140Y+R180*+S181*+T182S+E189P+N194F+L205Y+T224Y+Y242F+E259G+N279S+K280Y+G303R+E390A+G475K H1*+G7A+R37A+D118T+W140Y+R180*+S181*+T182S+E189P+N194F+L205Y+T224Y+Y242F+N279S+K280Y+G303R+E390A+G475K H1*+G7A+R37N+D118T+W140Y+R180*+S181*+T182S+E189P+N194F+L205Y+Y242F+E259G+M260D+N279S+K280Y+G303R+H420A+G475K H1*+N3D+G7A+D118T+W140Y+R180*+S181*+T182S+E189P+N194F+L205Y+Y242F+E259G+M260D+N279S+K280Y+G303R+E390A+G475K H1*+N3D+G7A+D118T+W140Y+R180*+S181*+T182S+E189P+N194F+L205Y+T224Y+Y242F+E259G+N279S+K280Y+G303R+E390A+G475K H1*+N3D+G7A+D118T+W140Y+R180*+S181*+T182S+E189P+N194F+L205Y+T224Y+Y242F+E259G+N279S+K280Y+G303R+E390A+G475K H1*+N3D+G7A+D118T+W140Y+R180*+S181*+T182S+E189P+N194F+L205Y+T224Y+Y242F+E259G+N279S+K280Y+G303R+E390A+G475K H1*+N3G+G7A+R37N+D118T+W140Y+R180*+S181*+T182S+E189P+N194F+L205Y+Y242F+E259G+N279S+K280Y+G303R+E390A+G475K H1*+N3D+G7A+D118T+W140Y+R180*+S181*+T182S+E189P+N194F+L205Y+Y242F+T256D+N279S+K280Y+G303R+E390A+G475K H1*+N3D+G7A+D118T+W140Y+R180*+S181*+T182S+E189P+N194F+L205Y+Y242F+T256D+N279S+K280Y+G303R+E390A+G475K H1*+N3D+G7A+D118T+W140Y+R180*+S181*+T182S+E189P+N194F+L205Y+Y242F+T256D+N279S+K280Y+G303R+E390A+G475K H1*+N3D+G7A+D118T+W140Y+R180*+S181*+T182S+E189P+N194F+L205Y+Y242F+T256D+N279S+K280Y+G303R+E390A+G475K H1*+G7A+D118T+W140Y+R180*+S181*+T182S+E189P+N194F+L205Y+Y242F+E259G+N279S+K280Y+S302T+E390A+N470M+G475K H1*+G7A+D118T+W140Y+R180*+S181*+T182S+E189P+N194F+L205Y+T224F+Y242F+E259G+N279S+K280Y+S302Y+E390A+G475K H1*+G7A+D118T+S132L+W140Y+R180*+S181*+T182S+E189P+N194F+L205Y+Y242F+E259G+N279S+K280Y+S302Y+E390A+G475K H1*+G7A+D118T+W140Y+R180*+S181*+T182S+E189P+N194F+L205Y+T224Y+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+G303R+E390A+H420A+G476A H1*+G7A+D118T+W140Y+R180*+S181*+T182S+E189P+N194F+L205Y+T224F+Y242F+E259G+N279S+K280Y+S302Y+E390A+G475K H1*+G7A+D118T+W140Y+R180*+S181*+T182S+E189P+N194F+L205Y+T224Y+Y242F+E259G+N279S+K280Y+S302Y+E390A+G475K H1*+G7A+D118T+W140Y+R180*+S181*+T182S+E189P+N194F+L205Y+Y242F+E259G+N279S+K280Y+S302Y+N394D+G475K H1*+G7A+D118T+W140Y+R180*+S181*+T182S+E189P+N194F+L205Y+T224F+Y242F+E259G+N279S+K280Y+G303R+E390A+H420A+G475K H1*+G7A+D118T+W140Y+R180*+S181*+T182S+E189P+N194F+L205Y+T224F+Y242F+E259G+N279S+K280Y+S302Y+E390A+G475K H1*+G7A+D118T+W140Y+R180*+S181*+T182S+E189P+N194F+L205Y+T224Y+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+S302Y+E390A+G475K H1*+G7A+D118T+S132D+W140Y+R180*+S181*+T182S+E189P+N194F+L205Y+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+S302T+N394W+G475K H1*+G7A+D118T+S132L+W140Y+R180*+S181*+T182S+E189P+N194F+L205Y+T224F+Y242F+E259G+N279S+K280Y+S302Y+E390A+G475K H1*+G7A+D118T+Q138V+W140Y+R180*+S181*+T182S+E189P+N194F+L205Y+Y242F+E259G+N279S+K280Y+S302Y+N394D+G475K H1*+G7A+D118T+S132L+W140Y+R180*+S181*+T182S+E189P+N194F+L205Y+Y242F+E259G+N279S+K280Y+S302Y+N394W+G475K H1*+N3D+G7A+D118T+W140Y+R180*+S181*+T182S+E189P+N194F+L205Y+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+G303R+E390A+H420A+G476H H1*+G7A+D118T+W140Y+R180*+S181*+T182S+E189P+N194F+L205Y+T224F+Y242F+E259G+N279S+K280Y+S302T+E390A+G475K H1*+G7A+D118T+W140Y+R180*+S181*+T182S+E189P+N194F+L205Y+Y242F+E259G+N279S+K280Y+S302T+N394D+G475K H1*+G7A+D118T+Q138V+W140Y+R180*+S181*+T182S+E189P+N194F+L205Y+Y242F+E259G+N279S+K280Y+S302Y+E390A+G475K H1*+G7A+D118T+Q138V+W140Y+R180*+S181*+T182S+E189P+N194F+L205Y+Y242F+E259G+N279S+K280Y+S302Y+E390A+G475K H1*+G7A+D118T+S132L+W140Y+R180*+S181*+T182S+E189P+N194F+L205Y+Y242F+E259G+N279S+K280Y+S302Y+N394W+G475K H1*+G7A+D118T+Q138V+W140Y+R180*+S181*+T182S+E189P+N194F+L205Y+T224F+Y242F+E259G+N279S+K280Y+S302Y+E390A+G475K H1*+G7A+Q98L+D118T+W140Y+R180*+S181*+T182S+E189P+N194F+L205Y+Y242F+E259G+N279S+K280Y+G303R+E390A+G475K H1*+G7A+R37N+D118T+W140Y+R180*+S181*+T182S+E189P+N194F+L205Y+Y242F+T256D+E259G+N279S+K280Y+G303R+E390A+G475K H1*+G7A+R37A+D118T+W140Y+R180*+S181*+T182S+E189P+N194F+L205Y+Y242F+E259G+N279S+K280Y+G303R+E390A+G475K H1*+G7A+R37N+D118T+W140Y+R180*+S181*+T182S+E189P+N194F+L205Y+Y242F+E259G+M260D+N279S+K280Y+G303R+N394D+G475K N3G+G7A+R37V+D118T+W140M+R180*+S181*+T182S+E189P+N194F+L205Y+Y242F+E259G+N279S+K280Y+G303R+E390A+H420A+G475K H1*+G7A+R37N+D118T+W140Y+R180*+S181*+T182S+E189P+N194F+L205Y+Y242F+E259G+N279S+K280Y+G303R+H323W+N394D+G475K H1*+G7A+R37A+D118T+W140Y+R180*+S181*+T182S+E189P+N194F+L205Y+Y242F+E259G+N279S+K280Y+G303R+P321Y+L354V+N394D+G475K H1*+G7A+R37N+D118T+W140Y+R180*+S181*+T182S+E189P+N194F+L205Y+Y242F+E259G+N279S+K280Y+G303R+H323W+Q360M+E390A+G475K H1*+N3D+G7A+D118T+W140Y+R180*+S181*+T182S+E189P+N194F+L205Y+Y242F+E259G+N279S+K280Y+G303R+H323W+E390A+G475K H1*+N3D+G7A+D118T+W140Y+R180*+S181*+T182S+E189P+N194F+L205Y+Y242F+E259G+N279S+K280Y+G303R+H323W+E390A+G475K H1*+G7A+D118T+W140Y+R180*+S181*+T182S+A185D+E189P+Y242F+E259G+F266Y+N279S+K280Y+G303R+E390A+G475K H1*+G7A+D118T+W140Y+R180Q+S181*+T182*+A185Q+E189P+Y242F+E259G+F266Y+N279S+K280Y+G303R+E390A+G475K H1*+G7A+D118T+W140Y+R180*+S181*+T182S+E189P+N194F+Y242F+E259G+N279S+K280Y+S302T+E390A+G475K H1*+G7A+D118T+W140Y+R180*+S181*+T182S+E189P+N194F+Y242F+E259G+M260D+N279S+K280Y+S302T+E390A+G475K H1*+G7A+R37N+D118T+W140Y+R180*+S181*+T182S+E189P+N194F+Y242F+E259G+N279S+K280Y+S302Y+E390A+G475K H1*+G7A+D118T+W140Y+R180*+S181*+T182S+E189P+N194F+Y242F+E259G+M260W+N279S+K280Y+G303R+E390A+G476A H1*+G7A+W140Y+R180*+S181*+E189P+N194F+Y242F+E259G+F266Y+N279S+K280Y+S302T+E390A+G475K H1*+G7A+D118T+W140Y+R180*+S181*+T182D+E189P+N194F+Y242F+E259G+N279S+K280Y+G303R+E390A+G476A H1*+G7A+D118T+S132L+W140Y+R180*+S181*+T182D+E189P+N194F+L205Y+Y242F+E259G+M260D+N279S+K280Y+G303R+E390A+G475K H1*+G7A+D118T+W140Y+R180*+S181*+T182D+E189P+N194F+Y242F+E259G+M260D+N279S+K280Y+S302T+E390A+G475K H1*+N3D+G7A+D118T+W140Y+R180*+S181*+T182D+E189P+N194F+Y242F+E259G+N279S+K280Y+S302T+E390A+H420A+G475K H1*+G7A+R37N+D118T+W140Y+R180*+S181*+T182D+E189P+N194F+Y242F+E259G+N279S+K280Y+S302Y+E390A+G475K H1*+G7A+D118T+W140Y+R180*+S181*+T182S+A185Q+E189P+N194F+L205Y+T224Y+Y242F+N279S+K280Y+G303R+E390A+G476A H1*+G7A+D118T+W140Y+R180*+S181*+T182S+A185Q+E189P+N194F+Y242F+E259G+M260D+N279S+K280Y+S302Y+E390A+N470M+G475K H1*+G7A+D118T+W140Y+R180*+S181*+T182S+A185Q+E189P+N194F+Y242F+E259G+N279S+K280Y+G303R+E390A+G476A H1*+G7A+D118T+W140Y+R180*+S181*+T182S+A185Q+E189P+N194F+Y242F+E259G+M260D+N279S+K280Y+S302T+E390A+G475K H1*+N3D+G7A+D118T+W140Y+R180*+S181*+T182S+A185Q+E189P+N194F+Y242F+E259G+N279S+K280Y+S302T+E390A+H420A+G475K H1*+G7A+R37N+D118T+W140Y+R180*+S181*+T182S+A185Q+E189P+N194F+Y242F+E259G+N279S+K280Y+S302Y+E390A+G475K H1*+G7A+D118T+W140Y+R180*+S181*+T182S+A185Q+E189P+N194F+L205Y+Y242F+E259G+M260W+N279S+K280Y+G303R+E390A+G476A H1*+G7A+D118T+W140Y+R180*+S181*+T182S+E189P+N194F+T224Y+Y242F+F266Y+N279S+K280Y+G303R+E390A+G476A H1*+G7A+D118T+W140Y+R180*+S181*+T182S+E189P+N194F+Y242F+E259G+M260D+F266Y+N279S+K280Y+S302Y+E390A+N470M+G475K H1*+G7A+D118T+W140Y+R180*+S181*+T182S+E189P+N194F+Y242F+E259G+F266Y+N279S+K280Y+S302T+E390A+G475K H1*+G7A+D118T+W140Y+R180*+S181*+T182S+E189P+N194F+Y242F+E259G+F266Y+N279S+K280Y+G303R+E390A+G476A H1*+G7A+D118T+S132L+W140Y+R180*+S181*+T182S+E189P+N194F+Y242F+E259G+M260D+F266Y+N279S+K280Y+G303R+E390A+G475K H1*+G7A+D118T+W140Y+R180*+S181*+T182S+E189P+N194F+Y242F+E259G+M260D+F266Y+N279S+K280Y+S302T+E390A+G475K H1*+G7A+D118T+W140Y+R180*+S181*+T182S+E189P+N194F+Y242F+E259G+M260W+F266Y+N279S+K280Y+G303R+E390A+G476A H1*+G7A+D118T+W140Y+R180*+S181*+T182D+E189P+N194F+Y242F+E259G+F266Y+N279S+K280Y+S302T+E390A+G475K H1*+G7A+D118T+W140Y+R180*+S181*+T182D+E189P+N194F+Y242F+E259G+M260D+F266Y+N279S+K280Y+S302T+E390A+G475K H1*+N3D+G7A+D118T+W140Y+R180*+S181*+T182D+E189P+N194F+Y242F+E259G+F266Y+N279S+K280Y+S302T+E390A+H420A+G475K H1*+G7A+R37N+D118T+W140Y+R180*+S181*+T182D+E189P+N194F+Y242F+E259G+F266Y+N279S+K280Y+S302Y+E390A+G475K H1*+G7A+D118T+W140Y+R180*+S181*+T182S+A185Q+E189P+N194F+T224Y+Y242F+F266Y+N279S+K280Y+G303R+E390A+G476A H1*+G7A+D118T+W140Y+R180*+S181*+T182S+A185Q+E189P+N194F+Y242F+E259G+F266Y+N279S+K280Y+S302T+E390A+G475K H1*+G7A+D118T+W140Y+R180*+S181*+T182S+A185Q+E189P+N194F+Y242F+E259G+F266Y+N279S+K280Y+G303R+E390A+G476A H1*+G7A+D118T+S132L+W140Y+R180*+S181*+T182S+A185Q+E189P+N194F+L205Y+Y242F+E259G+M260D+F266Y+N279S+K280Y+G303R+E390A+G475K H1*+N3D+G7A+D118T+W140Y+R180*+S181*+T182S+A185Q+E189P+N194F+Y242F+E259G+F266Y+N279S+K280Y+S302T+E390A+H420A+G475K H1*+G7A+D118T+W140Y+R180*+S181*+T182S+A185Q+E189P+N194F+Y242F+E259G+M260W+F266Y+N279S+K280Y+G303R+E390A+G476A H1*+G7A+W140Y+R180*+S181*+E189P+N194F+Y242F+E259G+M260D+N279S+K280Y+S302T+E390A+G475K H1*+N3D+G7A+W140Y+R180*+S181*+E189P+N194F+Y242F+E259G+F266Y+N279S+K280Y+S302T+E390A+H420A+G475K H1*+G7A+R37N+W140Y+R180*+S181*+E189P+N194F+Y242F+E259G+F266Y+N279S+K280Y+S302Y+E390A+G475K H1*+G7A+W140Y+R180*+S181*+E189P+N194F+Y242F+E259G+F266Y+N279S+K280Y+G303R+E390A+G476A H1*+G7A+D118T+W140Y+R180*+S181*+T182S+E189P+N194F+Y242F+E259G+M260D+N279S+K280Y+S302Y+E390A+N470M+G475K H1*+G7A+D118T+W140Y+R180*+S181*+T182D+E189P+N194F+Y242F+E259G+M260D+N279S+K280Y+S302Y+E390A+N470M+G475K H1*+G7A+D118T+W140Y+R180*+S181*+T182D+E189P+N194F+Y242F+E259G+M260W+N279S+K280Y+G303R+E390A+G476A H1*+G7A+D118T+W140Y+R180*+S181*+T182S+A185Q+E189P+N194F+Y242F+E259G+M260W+N279S+K280Y+G303R+E390A+G476A H1*+G7A+D118T+W140Y+R180*+S181*+T182S+E189P+N194F+T224Y+Y242F+N279S+K280Y+G303R+E390A+G476A H1*+G7A+D118T+W140Y+R180*+S181*+T182D+E189P+N194F+T224Y+Y242F+N279S+K280Y+G303R+E390A+G476A H1*+G7A+D118T+W140Y+R180*+S181*+T182S+A185Q+E189P+N194F+T224Y+Y242F+N279S+K280Y+G303R+E390A+G476A H1*+G7A+D118T+W140Y+R180*+S181*+T182S+A185Q+E189P+N194F+Y242F+E259G+N279S+K280Y+S302T+E390A+G475K H1*+G7A+D118T+W140Y+R180*+S181*+T182D+E189P+N194F+Y242F+E259G+F266Y+N279S+K280Y+G303R+E390A+G476A H1*+G7A+D118T+W140Y+R180*+S181*+T182D+E189P+N194F+Y242F+E259G+M260D+N279S+K280Y+S302Y+E390A+N470M+G475K H1*+G7A+D118T+W140Y+R180*+S181*+T182D+A185Q+E189P+N194F+Y242F+E259G+F266Y+N279S+K280Y+G303R+E390A+G475K H1*+G7A+D118T+W140Y+R180*+S181*+T182D+A185Q+Y242F+S243Q+E259G+F266Y+N279S+G303R+E390A+G475K H1*+G7A+D118T+W140Y+R180*+S181*+T182D+N194F+Y242F+S243Q+E259G+F266Y+N279S+G303R+E390A+G475K H1*+G7A+D118T+W140Y+R180*+S181*+T182S+A185Q+E189P+N196I+Y242F+E259G+F266Y+N279S+K280Y+G303R+E390A+G475K H1*+G7A+D118T+W140Y+R180*+S181*+T182S+A185D+E189P+Y242F+E259G+F266Y+N279S+K280Y+G303R+E390A+H420Y+G475K H1*+G7A+D118T+W140Y+R180*+S181*+A185Q+E189P+N194F+L205Y+Y242F+E259G+F266Y+N279S+K280Y+G303R+E390A+G475K H1*+G7A+W140Y+R180*+S181*+E189P+N194F+Y242F+E259G+M260W+N279S+K280Y+G303R+E390A+G476A H1*+G7A+D118T+W140Y+R180*+S181*+T182S+E189P+N194F+Y242F+E259G+M260D+N279S+K280Y+S302Y+E390A+N470M+G475K H1*+G7A+D118T+W140Y+R180*+S181*+T182D+E189P+N194F+T224Y+Y242F+N279S+K280Y+G303R+E390A+G476A H1*+G7A+D118T+W140Y+R180*+S181*+T182S+E189P+N194F+T224Y+Y242F+N279S+K280Y+G303R+E390A+G476A H1*+G7A+D118T+W140Y+R180*+S181*+T182D+A185Q+E189P+N194F+T224Y+Y242F+N279S+K280Y+G303R+E390A+G476A H1*+G7A+D118T+W140Y+R180*+S181*+T182D+A185Q+N194F+Y242F+E259G+N279S+K280Y+S302T+E390A+G475K H1*+G7A+R37N+D118T+W140Y+R180*+S181*+T182S+A185Q+E189P+N194F+L205Y+Y242F+E259G+N279S+K280Y+S302Y+E390A+G475K H1*+G7A+D118T+W140Y+R180*+S181*+T182S+A185Q+E189P+N194F+L205Y+Y242F+E259G+M260D+N279S+K280Y+S302T+E390A+G475K H1*+N3D+G7A+D118T+W140Y+R180*+S181*+T182D+E189P+N194F+L205Y+Y242F+E259G+N279S+K280Y+S302T+E390A+H420A+G475K H1*+G7A+W140Y+R180*+S181*+E189P+N194F+Y242F+E259G+M260D+N279S+K280Y+S302Y+E390A+N470A+G475K H1*+G7A+S132L+W140Y+R180*+S181*+E189P+N194F+Y242F+E259G+M260D+N279S+K280Y+G303R+E390A+G475K H1*+G7A+A31S+R37N+D118T+W140Y+R180*+S181*+T182S+A185Q+E189P+N194F+Y242F+E259G+F266Y+N279S+K280Y+S302Y+E390A+G475K H1*+G7A+D118T+W140Y+R180*+S181*+T182S+A185Q+E189P+N194F+Y242F+E259G+M260W+N279S+K280Y+G303R+E390A+G476A H1*+G7A+D118T+W140Y+R180*+S181*+T182D+E189P+N194F+L205Y+T224Y+Y242F+F266Y+N279S+K280Y+G303R+E390A+G476A H1*+G7A+D118T+W140Y+R180*+S181*+T182S+A185Q+E189P+N194F+Y242F+E259G+N279S+K280Y+S302T+E390A+G475K R180*+S181* T40N+F116L+D123N+N126D+N128Y+T131I+F155W+R180*+S181*+T182D+E189P+F202Y+K457N+P458S+G475K+G476E A60V+Q98L+G110K+R180*+S181*+A383D Q98L+G110K+S132D+Y135E+R180*+S181*+T315A G7H+Q98L+G110K+R180*+S181* G7H+W167P+R180*+S181*+G455R Q98L+V103A+G110K+R180*+S181* T114K+R180*+S181* E169V+R180*+S181* R180*+S181*+W188C W167D+R180*+S181* W167S+R180*+S181* W167E+R180*+S181* W167P+R180*+S181* R180*+S181*+E193I+H323N R180*+S181*+A203F W159L+R180*+S181* W159H+R180*+S181* K178A+R180*+S181* A111L+R180*+S181* G110K+R180*+S181* A111G+R180*+S181*, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID No: 1, 2, 3, 4, 5, 6, 7, 8, 9, 10, 11, 12, 13 or 14 and wherein the variant has alpha-amylase activity.

### Polynucleotides

The present invention also relates to polynucleotides encoding alpha-amylase variant(s) of the present invention.

In one aspect, the polynucleotide encoding alpha-amylase variants comprising a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475, 476, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

The techniques used to isolate or clone a polynucleotide are known in the art and include isolation from genomic DNA or cDNA, or a combination thereof. The cloning of the polynucleotides from genomic DNA can be effected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Trichoderma, Lecanicillium, Simplicillium, Aspergillus, Cornyascus, Acrophialophora, Rhinocladiella, Nemania, Talaromyces, Collariella, Rigidoporous, and/or Loramyces, or a related organism and thus, for example, may be a species variant of the polypeptide encoding region of the polynucleotide.

Modification of a polynucleotide encoding alpha-amylase variant(s) of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide encoding alpha-amylase variant(s) of the present invention.

In one aspect, the nucleic acid constructs comprising a polynucleotide encoding alpha-amylase variants comprising a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475, 476, using SEQ ID NO: 1 for numbering, wherein the polynucleotide is operably linked to one or more control sequences that direct the expression of the coding sequence in a suitable host cell under conditions compatible with the control sequences.

The present invention also relates to nucleic acid constructs comprising a polynucleotide encoding alpha-amylase variant(s) of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide which is recognized by a host cell for expression of the polynucleotide. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the polynucleotide of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is heterologous to the coding sequence. A heterologous signal peptide coding sequence may be required where the coding sequence does not naturally contain a signal peptide coding sequence. Alternatively, a heterologous signal peptide coding sequence may simply replace the natural signal peptide coding sequence to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiol. Rev. 57: 109-137.

Effective signal peptide coding sequences for filamentous fungal host cells are the signal peptide coding sequences obtained from the genes for Aspergillus niger neutral amylase, Aspergillus nigerglucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide encoding alpha-amylase variant(s) of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

In one aspect, the recombinant vectors comprising a polynucleotide encoding alpha-amylase variant comprising a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475, 476, using SEQ ID NO: 1 for numbering, a promoter, and transcriptional and translational stop signals. The various nucleotide and control sequences may be joined together to produce a recombinant expression vector that may include one or more (several) convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is a hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMB1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide encoding alpha-amylase variant(s) of the present invention operably linked to one or more control sequences that direct the production of a variant of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the variant and its source.

The host cell may be any cell useful in the recombinant production of alpha-amylase variant(s), e.g., a prokaryote or a eukaryote.

In some embodiments, the polypeptide is heterologous to the recombinant host cell.

In some embodiments, at least one of the one or more control sequences is heterologous to the polynucleotide encoding the alpha-amylase variant(s).

In some embodiments, the recombinant host cell comprises at least two copies, e.g., three, four, or five, of the polynucleotide of the present invention.

The host cell may be any microbial or plant cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 196I, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

Methods of Production of the Alpha-Amylase

The variant may be produced by a method comprising: (a) cultivating a host cell of the present invention under conditions suitable for the expression of the variant; and (b) recovering the variant.

In one aspect, the method of obtaining an alpha-amylase variant comprising: introducing into a parent alpha-amylase a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475, 476, using SEQ ID NO: 1 for numbering and the variant has alpha-amylase activity; and optionally recovering the variant.

The host cells are cultivated in a nutrient medium suitable for production of the hybrid or variant using methods known in the art. For example, the cell may be cultivated by shake flask cultivation, or small-scale or large-scale fermentation (including continuous, batch, fed-batch, or solid-state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the hybrid or variant is secreted into the nutrient medium, the hybrid or variant can be recovered directly from the medium. If the hybrid or variant is not secreted, it can be recovered from cell lysates.

The hybrid or variant may be detected using methods known in the art that are specific for the hybrid or variant. These detection methods may include use of specific antibodies, formation of an enzyme product, or disappearance of an enzyme substrate. For example, an enzyme assay may be used to determine the activity of the hybrid or variant.

In an alternative aspect, the hybrid or variant is not recovered, but rather a host cell as described herein expressing a hybrid or variant is used as a source of the hybrid or variant.

In one aspect, a method of improving wash performance of a variant of a polypeptide having alpha-amylase, said variant having the amino acid sequence of SEQ ID NO: 1, or having at least 60% sequence identity thereto, said method comprising the steps of: (a) a substitution and/or deletion of two, three or four positions in the polypeptide having alpha-amylase activity said positions corresponding to position 180, 181, 182, and 183 using SEQ ID NO: 1 for numbering; and (b) a modification at one or more positions corresponding to positions H1, H2, N3, G7, R26, A31, R37, T40, A41, N54, V56, A60, L63, K72, G73, K93, N94, Q98, V103, V104, M105, N106, A109, G110, A111, G113, T114, F116, V117, D118, D123, N126, N128, T131, S132, T134, Y135, Q136, I137, Q138, W140, K142, D144, P146, T151, Y152, F155, W159, Y160, T165, W167, E169, K172, L173, N174, I176, Y177, K178, R180, S181, T182, G183, A185, W188, E189, E193, N194, N196, F202, A203, L205, T224, N225, Y242, S243, F244, V252, T256, K258, E259, M260, F261, F266, N276, N279, K280, W283, S286, S302, G303, T315, H320, P321, M322, H323, W346, L354, E359, Q360, G361, Y362, V365, Y371, A383, E390, N394, F395, D405, I409, R414, H420, N422, T427, G455, K457, P458, N470, S472, V473, N474, G475 and G476, using SEQ ID NO: 1 for numbering and wherein said variant has alpha-amylase activity and improved (increased) wash performance compared to said polypeptide.

### Alpha-Amylase Variant Granules

The present invention also relates to enzyme granules/particles comprising the alpha-amylase variant(s) of the invention. In an embodiment, the granule comprises a core, and optionally one or more coatings (outer layers) surrounding the core.

The core may have a diameter, measured as equivalent spherical diameter (volume based average particle size), of 20-2000 μm, particularly 50-1500 μm, 100-1500 μm or 250-1200 μm.

In an embodiment, the core comprises one or more alpha-amylase variant of the present invention. In an embodiment, the core comprises one or more polypeptides having alpha-amylase variant of the present invention. In an embodiment, the core comprises one or more polypeptides having alpha-amylase variant and/or one or more polypeptides having alpha-amylase variant of the present invention.

The core may include additional materials such as fillers, fiber materials (cellulose or synthetic fibers), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

The core may include an inert particle with the enzyme absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA).

The coating may be applied in an amount of at least 0.1% by weight of the core, e.g., at least 0.5%, at least 1%, at least 5%, at least 10%, or at least 15%. The amount may be at most 100% 70%, 50%, 40% or 30%.

The coating is preferably at least 0.1 μm thick, particularly at least 0.5 μm, at least 1 μm or at least 5 μm. In some embodiments, the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit it is encapsulating/enclosing has few or none uncoated areas. The layer or coating should, in particular, be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

To provide acceptable protection, the salt coating is preferably at least 0.1 μm thick, e.g., at least 0.5 μm, at least 1 μm, at least 2 μm, at least 4 μm, at least 5 μm, or at least 8 μm. In a particular embodiment, the thickness of the salt coating is below 100 μm, such as below 60 μm, or below 40 μm.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular, having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminum. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular, alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710. Specific examples of suitable salts are NaCl(CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl(CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl(CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄(CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e. a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄.7H₂O), zinc sulfate heptahydrate (ZnSO₄.7H₂O), sodium phosphate dibasic heptahydrate (Na₂HPO₄.7H₂O), magnesium nitrate hexahydrate (Mg(NO₃)₂(6H₂O)), sodium citrate dihydrate and magnesium acetate tetrahydrate.

Preferably the salt is applied as a solution of the salt, e.g., using a fluid bed.

The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granule may optionally have one or more additional coatings. Examples of suitable coating materials are polyethylene glycol (PEG), methyl hydroxy-propyl cellulose (MHPC) and polyvinyl alcohol (PVA). Examples of enzyme granules with multiple coatings are described in WO 93/07263 and WO 97/23606.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in the Handbook of Powder Technology; Particle size enlargement by C. E. Capes; Volume 1; 1980; Elsevier. Preparation methods include known feed and granule formulation technologies, e.g.:

(a) Spray dried products, wherein a liquid enzyme-containing solution is atomized in a spray drying tower to form small droplets which during their way down the drying tower dry to form an enzyme-containing particulate material. Very small particles can be produced this way (Michael S. Showell (editor); Powdered detergents; Surfactant Science Series; 1998; vol. 71; page 140-142; Marcel Dekker).

(b) Layered products, wherein the enzyme is coated as a layer around a pre-formed inert core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in, e.g., WO 97/23606.

(c) Absorbed core particles, wherein rather than coating the enzyme as a layer around the core, the enzyme is absorbed onto and/or into the surface of the core. Such a process is described in WO 97/39116.

(d) Extrusion or pelletized products, wherein an enzyme-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme (Michael S. Showell (editor); Powdered detergents; Surfactant Science Series; 1998; vol. 71; pages 140-142; Marcel Dekker).

(e) Prilled products, wherein an enzyme-containing powder is suspended in molten wax and the suspension is sprayed, e.g., through a rotating disk atomizer, into a cooling chamber where the droplets quickly solidify (Michael S. Showell (editor); Powdered detergents; Surfactant Science Series; 1998; vol. 71; page 140-142; Marcel Dekker). The product obtained is one wherein the enzyme is uniformly distributed throughout an inert material instead of being concentrated on its surface. U.S. Pat. Nos. 4,016,040 and 4,713,245 describe this technique.

(f) Mixer granulation products, wherein an enzyme-containing liquid is added to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granulates comprising the enzyme. Such a process is described in U.S. Pat. No. 4,106,991 and related documents EP 170360, EP 304332, EP 304331, WO 90/09440 and WO 90/09428. In a particular product of this process, various high-shear mixers can be used as granulators. Granulates consisting of enzyme, fillers and binders etc. are mixed with cellulose fibers to reinforce the particles to produce a so-called T-granulate. Reinforced particles, are more robust, and release less enzymatic dust.

(g) Size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in Martin Rhodes (editor); Principles of Powder Technology; 1990; Chapter 10; John Wiley & Sons.

(h) Fluid bed granulation. Fluid bed granulation involves suspending particulates in an air stream and spraying a liquid onto the fluidized particles via nozzles. Particles hit by spray droplets get wetted and become tacky. The tacky particles collide with other particles and adhere to them to form a granule.

(i) The cores may be subjected to drying, such as in a fluid bed drier. Other known methods for drying granules in the feed or enzyme industry can be used by the skilled person. The drying preferably takes place at a product temperature of from 25 to 90° C. For some enzymes, it is important the cores comprising the enzyme contain a low amount of water before coating with the salt. If water sensitive enzymes are coated with a salt before excessive water is removed, it will be trapped within the core and may affect the activity of the enzyme negatively. After drying, the cores preferably contain 0.1-10% w/w water.

Non-dusting granulates may be produced, e.g., as disclosed in U.S. Pat. Nos. 4,106,991 and 4,661,452 and may optionally be coated by methods known in the art.

The granulate may further one or more additional enzymes. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of enzymes by the use of co-granulates is disclosed in WO 2013/188331.

The present invention also relates to protected enzymes prepared according to the method disclosed in EP 238,216.

In an embodiment, the granule further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof.

### Fermentation Broth Formulations or Cell Compositions

The present invention also relates to a fermentation broth formulation or a cell composition comprising alpha-amylase variant(s) of the present invention. The fermentation broth formulation or the cell composition further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In some embodiments, the fermentation broth formulation or the cell composition comprises a first organic acid component comprising at least one 1-5 carbon organic acid and/or a salt thereof and a second organic acid component comprising at least one 6 or more carbon organic acid and/or a salt thereof. In some embodiments, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In some embodiments, the killed cells and/or cell debris are removed from a cell-killed whole broth to provide a composition that is free of these components.

The fermentation broth formulation or cell composition may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell composition of the present invention may be produced by a method described in WO 90/15861 or WO 2010/096673.

### Cleaning Compositions

The cleaning composition of the present invention preferably relates to products for and/or methods relating to and/or use of the claimed compositions that are for air care, car care, dish-washing, laundry cleaning, care, (conditioning and/or softening), laundry and rinse additives, hard surface cleaning and/or treatment, and other cleaning for consumer or institutional use. Preferably the composition comprises a laundry detergent or hard surface cleaning composition, such as a dish-washing composition. The composition of the invention may be a solid (for example, a bar, tablet, powder, granules), liquid, gel and/or paste or may be in the form of a sheet. A preferred form is a unit dose form which may be a tablet, sheet, or preferably a pouch comprising liquid and/or solid compositions. Preferred are multi-compartment pouches. The composition may be a powder-form all-purpose “heavy-duty” washing agent, a paste-form all-purpose, a heavy-duty liquid type, a liquid fine-fabric, a hand dishwashing agent, a light duty dishwashing agent, a high-foaming type, a machine dishwashing agent. The dishwashing composition may be in the form of a liquid, gel or powder and it may be in the form of a unit dose, such as a tablet or pouch, and is preferably a main wash composition or a rinse-aid type. The composition can also be in unit dose packages, including those known in the art and those that are water soluble, water insoluble and/or water permeable. A liquid detergent may be aqueous, typically containing up to 70% water and 0-30% organic solvent, or non-aqueous or a solution containing more than 0.5 g/L of the detergent composition.

The composition of the invention may for example be formulated as a hand or machine laundry detergent composition including a laundry additive composition suitable for pre-treatment of stained fabrics and a rinse added fabric softener composition, or be formulated as a detergent composition for use in general household hard surface cleaning operations, or be formulated for hand or machine dishwashing operations.

Preferred unit dose compositions comprise solid (preferably in the form of a powder), or liquid or a combination thereof, preferably in a multi-compartment unit dose. Especially preferred is a unit dose form comprising liquid detergent encapsulated in water-soluble material in the form of a pouch, optionally in the form of a multi-component pouch. A preferred unit dose comprises liquid laundry detergent/care composition, optionally in a unit dose form.

The cleaning composition of the invention may be in the form of soap bar, preferably a laundry soap bar and may be used for hand washing laundry, fabrics and/or textiles. The term laundry soap bar includes laundry bars, soap bars, combo bars, syndet bars and detergent bars.

The cleaning compositions, in addition to the hybrid polypeptide or amylase variant enzyme, comprise a cleaning/detergent adjunct. The expression cleaning adjunct herein typically comprises a combination of more than one cleaning adjunct. Typically the cleaning adjunct will be present in the composition in an amount from 0.0001 to 99.9999 wt %, preferably from 10 to 99.999 wt %, preferably at least 30 or 40 or 50 wt % cleaning adjunct. Suitable cleaning adjuncts comprise: surfactants, builders, bleaches, bleach catalysts, colorants, bleach boosters, chelating agents, dye transfer agents, deposition aids, dispersants, additional enzymes, and enzyme stabilizers, catalytic materials, bleach activators, hydrogen peroxide, sources of hydrogen peroxide, optical brighteners, photoactivators, fluorescers, fabric hueing agents, fabric conditioners, preformed peracids, polymeric dispersing agents, clay soil removal/anti-redeposition agents, filler salts, hydrotropes, brighteners, suds suppressors, structure elasticizing agents, fabric softeners, hydrolyzable surfactants, preservatives, anti-oxidants, anti-shrinkage agents, germicides, fungicides, anti-tarnish, anti-corrosion agents, alkalinity sources, solubilizing agents, carriers, processing aids, pigments, dyes, perfumes and pH control agents, encapsulates, polymers and mixtures thereof. For example, these may include: bleach ingredients such as imine bleach boosters; sources of hydrogen peroxide such as percarbonate and/or perborate, especially percarbonate coated with material such as carbonate and/or sulphate salt, silicate salt, borosilicate, and any mixture thereof; pre-formed peracid, including pre-formed peracid in encapsulated form; transition metal catalysts; suds suppressors or suppressor systems such as silicone based suds suppressors and/or fatty acid based suds suppressors; fabric-softeners such as clay, silicone and/or quaternary ammonium compounds; flocculants such as polyethylene oxide; dye transfer inhibitors such as polyvinylpyrrolidone, poly 4-vinylpyridine N-oxide and/or co-polymer of vinylpyrrolidone and vinylimidazole; fabric integrity components such as oligomers produced by the condensation of imidazole and epichlorhydrin; soil dispersants and soil anti-redeposition aids such as alkoxylated polyamines and ethoxylated ethyleneimine polymers; anti-redeposition components such as polyesters; carboxylate polymers such as maleic acid polymers or co-polymers of maleic and acrylic acid; perfumes such as perfume microcapsules, starch encapsulated accords, perfume spray-on; soap rings; aesthetic particles; dyes; fillers such as sodium sulphate and/or citrus fibres, although it may be preferred for the composition to be substantially free of fillers; silicate salt such as sodium silicate, including 1.6R and 2.0R sodium silicate, or sodium metasilicate; co-polyesters of di-carboxylic acids and diols; cellulosic polymers such as methyl cellulose, carboxymethyl cellulose, hydroxyethoxycellulose, or other alkyl or alkylalkoxy cellulose; solvents such as 1,2 propanediol, monoethanolamine; diethylene glycol, ethanol, and any mixture thereof; hydrotropes such as sodium cumene sulphonate, sodium xylene sulphonate, sodium toluene sulphonate, and any mixtures; organic acids such as citric acid; and any combination thereof. Preferably the composition comprises a cleaning adjunct comprising surfactant and additional enzyme. Preferably the composition comprises one or more adjuncts selected from the group consisting of (i) perfume microcapsule; (ii) fabric hueing agent; (iii) protease; (iv) amphiphilic cleaning polymer; (v) lipase, (vi) DNase and/or RNase, (vii) mannanase, (viii) xanthan lyase; (ix) hexosaminidase; or (viii) mixtures thereof.

Preferably the composition comprises a surfactant, preferably from 0.1 to 60 weight % or from 0.5 to 50 wt % or 1 to 40 wt % of the composition, surfactant. The surfactant preferably comprises a surfactant system comprising a mixture of more than one surfactant, which may comprise for example, anionic, non-ionic including semi-polar, cationic, zwitterionic and/or amphoteric surfactants and mixtures thereof.

Preferably the composition comprises an anionic surfactant. Preferred anionic surfactants are sulfonate and sulfate surfactants, preferably alkylbenzene sulphonates and/or (optionally alkoxylated) alkyl sulfates. Particularly preferred anionic surfactant comprises linear alkylbenzenesulfonates (LAS). Preferred alkyl sulfates comprise alkyl ether sulfates, especially C-9-15 alcohol ether sulfates, especially those having an average degree of ethoxylation from 0.5 to 7, preferably from 1 to 5, C8-C16 ester sulfates and C10-C14 ester sulfates, such as mono dodecyl ester sulfates. In a preferred composition according to the invention the surfactant comprises anionic surfactant, preferably comprising alkyl benzene sulphonate and/or optionally ethoxylated alkyl sulfate, preferably having a degree of ethoxylation from 0 to 7, more preferably from 0.5 to 3. Isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ether sulfates (AES or AEOS or FES, also known as alcohol ethoxy sulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof are also suitable anionic surfactants. In a preferred embodiment the surfactant comprises anionic surfactant, preferably comprising alkyl benzene sulphonate and/or optionally ethoxylated alkyl sulfate, preferably having a degree of ethoxylation from 0 to 7, more preferably from 0.5 to 3.

The anionic surfactants are preferably added to the cleaning composition in the form of salts. Preferred cations are alkali metal ions, such as sodium and potassium. However, the salt form of the anionic surfactant may be formed in situ by neutralization of the acid form of the surfactant with alkali such as sodium hydroxide or an amine, such as mono-, di-, or tri-ethanolamine. Preferably the surfactant comprises non-ionic surfactant. Preferably the surfactant comprises an anionic and a nonionic surfactant, preferably in a weight ratio of anionic to nonionic of from 30:1 to 1:2, preferably from 20:1 to 2:3 or 1:1.

Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof. Alcohol ethoxylates are particularly preferred, preferably having a C9-18 alkyl chain, preferably from C12-15 and preferably having an average degree of ethoxylation 3 to 9, more preferably from 3 to 7. Commercially available nonionic surfactants include Plurafac™, Lutensol™ and Pluronic™ from BASF, Dehypon™ series from Cognis and Genapol™ series from Clariant.

The cleaning composition preferably comprises from about 1% to about 40% of an anionic surfactant. The cleaning composition preferably comprises from 0.2% to about 40% of a non-ionic surfactant such as alcohol ethoxylate, nonyl-phenol ethoxylate, alkylpolyglycoside, alkyldimethylamine-oxide, ethoxylated fatty acid monoethanol-amide, fatty acid monoethanolamide, polyhydroxy alkyl fatty acid amide, or N-acyl N-alkyl derivatives of glucosamine (“glucamides”).

The cleaning composition preferably comprises one or more additional enzymes. Therefore a preferred composition comprises (a) amylase as defined herein, and (b) one or more additional enzymes preferably selected from the group consisting of aminopeptidase, amylase, carbohydrase, carboxypeptidase, catalase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, haloperoxidase, haloperoxygenase, hexosaminidase, invertase, laccase, licheninase, lipase, mannanase, mannosidase, oxidase, pectinases, pectin lyases, pectinolytic enzyme, peptidoglutaminase, peroxidase, phytase, polyphenoloxidase, proteolytic enzyme (protease), peroxidase, ribonuclease, transglutaminase, xylanase, xanthan lyase, xanthanase, xyloglucanase and mixtures thereof. Preferably the composition comprises additional enzymes selected from xanthan lyase, xanthanase, mannanase and mixtures thereof. Mannanase is particularly preferred. Xanthan lyase and xanthanase and mixtures thereof are also particularly preferred. Proteases, lipases and/or cellulases are preferred, particularly proteases. The additional enzyme(s) may be produced, for example, by a microorganism belonging to the genus Aspergillus, e.g., Aspergillus aculeatus, Aspergillus awamori, Aspergillus foetidus, Aspergillus fumigatus, Aspergillus japonicus, Aspergillus nidulans, Aspergillus niger, or Aspergillus oryzae; Fusarium, e.g., Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sulphureum, Fusarium toruloseum, Fusarium trichothecioides, or Fusarium venenatum; Humicola, e.g., Humicola insolens or Humicola lanuginosa; or Trichoderma, e.g., Trichoderma harzianum, Trichoderma koningii, Trichoderma longibrachiatum, Trichoderma reesei, or Trichoderma viride.

Preferably the composition comprises one or more, of one or more of: a protease, a lipase, a peroxidase, additional amylolytic enzyme, e.g., an additional alpha-amylase, glucoamylase, isoamylase, maltogenic amylase, preferably an additional alpha-amylase, CGTase, cellulase, mannanase (such as MANNAWAY™ from Novozymes, Denmark), pectinase, pectate lyase, cutinase, and/or laccase or mixtures of more than one of these.

In general the properties of the chosen enzyme(s) should be compatible with the selected detergent, (i.e., pH-optimum, compatibility with other enzymatic and non-enzymatic ingredients, etc.), and the enzyme(s) should be present in effective amounts. Preferably, the composition of the invention comprises at least 0.01 mg, preferably from about 0.05 to about 10, more preferably from about 0.1 to about 6, especially from about 0.2 to about 5 mg of active further enzyme/g of composition.

Proteases: Suitable proteases include metalloproteases and/or serine proteases, including neutral or alkaline microbial serine proteases, such as subtilisins (EC 3.4.21.62). Suitable proteases include those of animal, vegetable or microbial origin. In one aspect, such suitable protease may be of microbial origin. The suitable proteases include chemically or genetically modified mutants of the aforementioned suitable proteases. In one aspect, the suitable protease may be a serine protease, such as an alkaline microbial protease or/and a trypsin-type protease. Examples of suitable neutral or alkaline proteases include:

(a) subtilisins (EC 3.4.21.62), including those derived from Bacillus, such as Bacillus lentus, B. alkalophilus, B. subtilis, B. amyloliquefaciens, Bacillus pumilus and Bacillus gibsonii described in U.S. Pat. No. 6,312,936 B1, U.S. Pat. Nos. 5,679,630, 4,760,025, 7,262,042 and WO09/021867.

(b) trypsin-type or chymotrypsin-type proteases, such as trypsin (e.g., of porcine or bovine origin), including the Fusarium protease described in WO 89/06270 and the chymotrypsin proteases derived from Cellumonas described in WO 05/052161 and WO 05/052146.

(c) metalloproteases, including those derived from Bacillus amyloliquefaciens described in WO 07/044993A2.

Preferred proteases include those derived from Bacillus gibsonii or Bacillus Lentus.

Suitable commercially available protease enzymes include those sold under the trade names Alcalase®, Savinase®, Primase®, Durazym®, Polarzyme®, Kannase®, Liquanase®, Liquanase Ultra®, Savinase Ultra®, Ovozyme®, Neutrase®, Everlase® and Esperase® by Novozymes A/S (Denmark), those sold under the tradename Maxatase®, Maxacal®, Maxapem®, Properase®, Purafect®, Purafect Prime®, Purafect Ox®, FN3®, FN4®, Excellase® and Purafect OXP® by Genencor International, those sold under the tradename Opticlean® and Optimase® by Solvay Enzymes, those available from Henkel/Kemira, namely BLAP (sequence shown in FIG. 29 of U.S. Pat. No. 5,352,604 with the folowing mutations S99D+S101 R+S103A+V104I+G159S, hereinafter referred to as BLAP), BLAP R (BLAP with S3T+V41+V199M+V205I+L217D), BLAP X (BLAP with S3T+V41+V205I) and BLAP F49 (BLAP with S3T+V41+A194P+V199M+V205I+L217D)—all from Henkel/Kemira; and KAP (Bacillus alkalophilus subtilisin with mutations A230V+S256G+S259N) from Kao. Further suitable proteases are described in WO2011/03623, WO2011/140316, WO2011/140364 and WO2012/05778.

Lipases: Suitable lipases include those of bacterial or fungal origin. Chemically modified or protein engineered mutants are included. Examples of useful lipases include lipases from Humicola (synonym Thermomyces), e.g., from H. lanuginosa (T. lanuginosus) or from H. insolens, a Pseudomonas lipase, e.g., from P. alcaligenes or P. pseudoalcaligenes, P. cepacia P. stutzeri, P. fluorescens, Pseudomonas sp. strain SD 705, P. wisconsinensis, a Bacillus lipase, e.g., from B. subtilis (Dartois et al. (1993), Biochemica et Biophysica Acta, 1131, 253-360), B. stearothermophilus or B. pumilus.

The lipase may be a “first cycle lipase” such as those described in U.S. Pat. No. 6,939,702 B1 and US PA 2009/0217464. In one aspect, the lipase is a first-wash lipase, preferably a variant of the wild-type lipase from Thermomyces lanuginosus comprising T231R and N233R mutations. The wild-type sequence is the 269 amino acids (amino acids 23-291) of the Swissprot accession number Swiss-Prot O59952 (derived from Thermomyces lanuginosus (Humicola lanuginosa)). Preferred lipases would include those sold under the tradenames Lipex®, Lipolex® and Lipoclean®.

Cellulases: Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum.

In one aspect, preferred enzymes include microbial-derived endoglucanases exhibiting endo-beta-1,4-glucanase activity (E.C. 3.2.1.4), preferably selected from the group comprising:
(a) a bacterial polypeptide endogenous to a member of the genus Bacillus which has a sequence of at least 90%, 94%, 97% and even 99% identity to the amino acid sequence SEQ ID NO:2 in U.S. Pat. No. 7,141,403B2;
(b) a glycosyl hydrolase having enzymatic activity towards both xyloglucan and amorphous cellulose substrates, wherein the glycosyl hydrolase is selected from GH families 5, 12, 44 or 74;
(c) a glycosyl hydrolase having a sequence of at least 90%, 94%, 97% and even 99% identity to the amino acid sequence SEQ ID NO:3 in WO09/148983;
(d) and mixtures thereof.

Suitable endoglucanases are sold under the tradenames Celluclean® and Whitezyme® (Novozymes A/S, Bagsvaerd, Denmark).

Other commercially available cellulases include CELLUZYME®, and CAREZYME® (Novozymes A/S), CLAZINASE®, and PURADAX HA® (Genencor International Inc.), and KAC-500(B)® (Kao Corporation).

Additional amylases: Preferably the composition comprises an additional (further) amylase. Suitable further amylases include alpha-amylases including those of bacterial or fungal origin. Chemically or genetically modified mutants (variants) are included. A preferred alkaline alpha-amylase is derived from a strain of Bacillus, such as Bacillus licheniformis, Bacillus amyloliquefaciens, Bacillus stearothermophilus, Bacillus subtilis, or other Bacillus sp., such as Bacillus sp. NCBI 12289, NCBI 12512, NCBI 12513, DSM 9375 (U.S. Pat. No. 7,153,818) DSM 12368, DSMZ no. 12649, KSM AP1378 (WO 97/00324), KSM K36 or KSM K38 (EP 1,022,334). Preferred further amylases may be selected from: (a) variants described in WO 94/02597, WO 94/18314, WO96/23874 and WO 97/43424, especially the variants with substitutions in one or more of the following positions versus the enzyme listed as SEQ ID No. 2 in WO 96/23874: 15, 23, 105, 106, 124, 128, 133, 154, 156, 181, 188, 190, 197, 202, 208, 209, 243, 264, 304, 305, 391, 408, and 444; (b) variants described in WO 96/23873, WO00/60060, WO06/002643 and WO2017/192657, especially the variants with one or more substitutions in the following positions versus the AA560 enzyme listed as SEQ ID No. 12 in WO 06/002643: 26, 30, 33, 82, 37, 106, 118, 128, 133, 149, 150, 160, 178, 182, 186, 193, 203, 214, 231, 246, 256, 257, 258, 269, 270, 272, 283, 295, 296, 298, 299, 303, 304, 305, 311, 314, 315, 318, 319, 339, 345, 361, 378, 383, 419, 421, 437, 441, 444, 445, 446, 447, 450, 461, 471, 482, 484, preferably that also contain the deletions of D183* and G184*; (c) variants exhibiting at least 90% identity with SEQ ID No. 4 in WO06/002643, the wild-type enzyme from Bacillus SP722, especially variants with deletions in the 183 and 184 positions and variants described in WO 00/60060, which is incorporated herein by reference; (d) variants exhibiting at least 95% identity with the wild-type enzyme from Bacillus sp.707 (SEQ ID NO:7 in U.S. Pat. No. 6,093,562), especially those comprising one or more of the following mutations M202, M208, S255, R172, and/or M261. Preferably said amylase comprises one or more of M202L, M202V, M202S, M202T, M202I, M202Q, M202W, S255N and/or R172Q. Particularly preferred are those comprising the M202L or M202T mutations; (e) variants described in WO 09/149130, preferably those exhibiting at least 90% identity with SEQ ID NO: 1 or SEQ ID NO:2 in WO 09/149130, the wild-type enzyme from Geobacillus Stearothermophilus or a truncated version thereof; (f) variants exhibiting at least 89% identity with SEQ ID NO:1 in WO2016091688, especially those comprising deletions at positions H183+G184 and additionally one or more mutations at positions 405, 421, 422 and/or 428; (g) variants exhibiting at least 60% amino acid sequence identity with the “PcuAmyl α-amylase” from Paenibacillus curdlanolyticus YK9 (SEQ ID NO:3 in WO2014099523); (h) variants exhibiting at least 60% amino acid sequence identity with the “CspAmy2 amylase” from Cytophaga sp. (SEQ ID NO:1 in WO2014164777); (i) variants exhibiting at least 85% identity with AmyE from Bacillus subtilis (SEQ ID NO:1 in WO2009149271); (j) variants exhibiting at least 90% identity with the wild-type amylase from Bacillus sp. KSM-K38 with accession number AB051102; (k) variants exhibiting at least 80% identity with the mature amino acid sequence of AAI10 from Bacillus sp (SEQ ID NO:7 in WO2016180748); (l) variants exhibiting at least 80% identity with the mature amino acid sequence of Alicyclobacillus sp. amylase (SEQ ID NO:8 in WO2016180748); or mixtures thereof. Where present, the composition of the invention preferably comprises from at least 0.01 mg, preferably from about 0.05 to about 10, more preferably from about 0.1 to about 6, especially from about 0.2 to about 5 mg of active further amylase/g of composition.

Suitable commercially available alpha-amylases include DURAMYL®, LIQUEZYME®, TERMAMYL®, TERMAMYL ULTRA®, NATALASE®, SUPRAMYL®, STAINZYME®, STAINZYME PLUS®, FUNGAMYL®, ATLANTIC®, INTENSA® and BAN® (Novozymes A/S, Bagsvaerd, Denmark), KEMZYM® AT 9000 Biozym Biotech Trading GmbH Wehlistrasse 27b A-1200 Wien Austria, RAPIDASE®, PURASTAR®, ENZYSIZE®, OPTISIZE HT PLUS®, POWERASE®, PREFERENZ S® series (including PREFERENZ S1000® and PREFERENZ S2000® and PURASTAR OXAM® (DuPont., Palo Alto, Calif.) and KAM® (Kao, 14-10 Nihonbashi Kayabacho, 1-chome, Chuo-ku Tokyo 103-8210, Japan). In one aspect, suitable amylases include ATLANTIC®, STAINZYME®, POWERASE®, INTENSA® and STAINZYME PLUS® and mixtures thereof.

Peroxidases/Oxidases: Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257.

Commercially available peroxidases include GUARDZYME® (Novozymes A/S).

Other enzymes: Other preferred enzymes include pectate lyases sold under the tradenames Pectawash®, Pectaway® and mannanases sold under the tradenames Mannaway® (all from Novozymes A/S, Bagsvaerd, Denmark), and Purabrite® (Genencor International Inc., Palo Alto, Calif.).

The enzyme(s) may be included in the cleaning composition by adding separate additives (pre-mixes) containing one or more enzymes, or by adding a combined additive (pre-mix) comprising all of these enzymes. A detergent additive of the invention, i.e., a separate additive or a combined additive, can be formulated, e.g., granulate, a liquid, a slurry, etc. Preferred detergent additive formulations are granulates, in particular non-dusting granulates, liquids, in particular stabilized liquids, or slurries.

Non-dusting granulates may be produced and may optionally be coated by methods known in the art. Examples of waxy coating materials are poly(ethylene oxide) products (polyethyleneglycol, PEG) with mean molar weights of 1000 to 20000; ethoxylated nonyl-phenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Film-forming coating materials may be applied for example by fluid bed techniques. Liquid enzyme preparations may, for instance, be stabilized by adding a polyol such as propylene glycol, a sugar or sugar alcohol, lactic acid or boric acid according to established methods.

The cleaning composition may comprise a fabric hueing agent (sometimes referred to as shading, bluing or whitening agents). Typically the hueing agent provides a blue or violet shade to fabric. Hueing agents can be used either alone or in combination to create a specific shade of hueing and/or to shade different fabric types. This may be provided for example by mixing a red and green-blue dye to yield a blue or violet shade. Hueing agents may be selected from any known chemical class of dye, including but not limited to acridine, anthraquinone (including polycyclic quinones), azine, azo (e.g., monoazo, disazo, trisazo, tetrakisazo, polyazo), including premetallized azo, benzodifurane and benzodifuranone, carotenoid, coumarin, cyanine, diazahemicyanine, diphenylmethane, formazan, hemicyanine, indigoids, methane, naphthalimides, naphthoquinone, nitro and nitroso, oxazine, phthalocyanine, pyrazoles, stilbene, styryl, triarylmethane, triphenylmethane, xanthenes and mixtures thereof, preferred dyes are azo, especially bis azo, anthraquinone, azine and triarylmethane dyes. Azo dyes are especially preferred.

Suitable fabric hueing agents include dyes, dye-clay conjugates, and organic and inorganic pigments. Suitable dyes include small molecule dyes and polymeric dyes; polymeric dyes are preferred. Suitable small molecule dyes include small molecule dyes selected from the group consisting of dyes falling into the Colour Index (C.I.) classifications of Direct, Basic, Reactive or hydrolysed Reactive, Solvent or Disperse dyes for example that are classified as Blue, Violet, Red, Green or Black, and provide the desired shade either alone or in combination. In another aspect, suitable small molecule dyes include small molecule dyes selected from the group consisting of Colour Index (Society of Dyers and Colourists, Bradford, UK) numbers Direct Violet dyes such as 9, 35, 48, 51, 66, and 99, Direct Blue dyes such as 1, 71, 80 and 279, Acid Red dyes such as 17, 73, 52, 88 and 150, Acid Violet dyes such as 15, 17, 24, 43, 49 and 50, Acid Blue dyes such as 15, 17, 25, 29, 40, 45, 75, 80, 83, 90 and 113, Acid Black dyes such as 1, Basic Violet dyes such as 1, 3, 4, 10 and 35, Basic Blue dyes such as 3, 16, 22, 47, 66, 75 and 159, Disperse or Solvent dyes such as those described in EP1794275 or EP1794276, or dyes as disclosed in U.S. Pat. No. 7,208,459 B2, and mixtures thereof. In another aspect, suitable small molecule dyes include small molecule dyes selected from the group consisting of Colour Index numbers Acid Violet 17, Direct Blue 71, Direct Violet 51, Direct Blue 1, Acid Red 88, Acid Red 150, Acid Blue 29, Acid Blue 113 or mixtures thereof.

Suitable polymeric dyes include polymeric dyes selected from the group consisting of polymers containing covalently bound (sometimes referred to as conjugated) chromogens, (dye-polymer conjugates), for example polymers with chromogens co-polymerized into the backbone of the polymer and mixtures thereof. Polymeric dyes include those described in WO2011/98355, WO2011/47987, US2012/090102, WO2010/145887, WO2006/055787 and WO2010/142503.

In another aspect, suitable polymeric dyes include polymeric dyes selected from the group consisting of fabric-substantive colorants sold under the name of Liquitint® (Milliken, Spartanburg, S.C., USA), dye-polymer conjugates formed from at least one reactive dye and a polymer selected from the group consisting of polymers comprising a moiety selected from the group consisting of a hydroxyl moiety, a primary amine moiety, a secondary amine moiety, a thiol moiety and mixtures thereof. In still another aspect, suitable polymeric dyes include polymeric dyes selected from the group consisting of Liquitint® Violet CT, carboxymethyl cellulose (CMC) covalently bound to a reactive blue, reactive violet or reactive red dye such as CMC conjugated with C.I. Reactive Blue 19, sold by Megazyme, Wicklow, Ireland under the product name AZO-CM-CELLULOSE, product code S-ACMC, alkoxylated triphenyl-methane polymeric colourants, alkoxylated thiophene polymeric colourants, and mixtures thereof.

Preferred hueing dyes include the alkoxylated thiophene azo whitening agents, for example as described in US2008/0177090 which may be optionally anionic, such as those selected from Examples 1-42 in Table 5 of WO2011/011799. Other preferred dyes are disclosed in U.S. Pat. No. 8,138,222.

Suitable pigments include pigments selected from the group consisting of flavanthrone, indanthrone, chlorinated indanthrone containing from 1 to 4 chlorine atoms, pyranthrone, dichloropyranthrone, monobromodichloropyranthrone, dibromodichloropyranthrone, tetrabromopyranthrone, perylene-3,4,9,10-tetracarboxylic acid diimide, wherein the imide groups may be unsubstituted or substituted by C1-C3-alkyl or a phenyl or heterocyclic radical, and wherein the phenyl and heterocyclic radicals may additionally carry substituents which do not confer solubility in water, anthrapyrimidinecarboxylic acid amides, violanthrone, isoviolanthrone, dioxazine pigments, copper phthalocyanine which may contain up to 2 chlorine atoms per molecule, polychloro-copper phthalocyanine or polybromochloro-copper phthalocyanine containing up to 14 bromine atoms per molecule, Ultramarine Blue (C.I. Pigment Blue 29), Ultramarine Violet (C.I. Pigment Violet 15) and mixtures thereof.

Builders—The cleaning composition may further contain builders, such as builders based on carbonate, bicarbonate or silicates which may be Zeolites, such as Zeolite A, Zeolite MAP (Maximum Aluminium type P). Zeolites, useable in laundry preferably has the formula Na₁₂(AlO₂)₁₂(SiO₂)₁₂.27H₂O and the particle size is usually between 1-10 μm forzeolite A and 0.7-2 um for zeolite MAP. Other builders are Sodium metasilicate (Na₂SiO₃.nH₂O or Na₂Si₂O₅.nH₂O) strong alkaline and preferably used in dish wash. In preferred embodiments, the amount of a detergent builder may be above 5%, above 10%, above 20%, above 30%, above 40% or above 50%, and may be below 80%, 65%. In a dishwash detergent, the level of builder is typically 40-65%, particularly 50-65% or even 75-90%.

Encapsulates—The composition may comprise an encapsulate. In one aspect, an encapsulate comprising a core, a shell having an inner and outer surface, said shell encapsulating said core.

In one aspect of said encapsulate, said core may comprise a material selected from the group consisting of perfumes; brighteners; dyes; insect repellants; silicones; waxes; flavors; vitamins; fabric softening agents; skin care agents in one aspect, paraffins; enzymes; anti-bacterial agents; bleaches; sensates; and mixtures thereof; and said shell may comprise a material selected from the group consisting of polyethylenes; polyamides; polystyrenes; polyisoprenes; polycarbonates; polyesters; polyacrylates; aminoplasts, in one aspect said aminoplast may comprise a polyureas, polyurethane, and/or polyureaurethane, in one aspect said polyurea may comprise polyoxymethyleneurea and/or melamine formaldehyde; polyolefins; polysaccharides, in one aspect said polysaccharide may comprise alginate and/or chitosan; gelatin; shellac; epoxy resins; vinyl polymers; water insoluble inorganics; silicone; and mixtures thereof.

In one aspect of said encapsulate, said core may comprise perfume. Such encapsulates are perfume microcapsules. Suitable encapsulate shell may comprise melamine formaldehyde and/or cross linked melamine formaldehyde, and/or polyacrylate.

In one aspect, at least 75%, 85% or even 90% of said encapsulates may have a particle size of from about 1 microns to about 80 microns, about 5 microns to 60 microns, from about 10 microns to about 50 microns, or even from about 15 microns to about 40 microns.

In one aspect, at least 75%, 85% or even 90% of said encapsulates may have a particle wall thickness of from about 30 nm to about 250 nm, from about 80 nm to about 180 nm, or even from about 100 nm to about 160 nm.

In one aspect, said encapsulates' core material may comprise a material selected from the group consisting of a perfume raw material and/or optionally a material selected from the group consisting of vegetable oil, including neat and/or blended vegetable oils including caster oil, coconut oil, cottonseed oil, grape oil, rapeseed, soybean oil, corn oil, palm oil, linseed oil, safflower oil, olive oil, peanut oil, coconut oil, palm kernel oil, castor oil, lemon oil and mixtures thereof; esters of vegetable oils, esters, including dibutyl adipate, dibutyl phthalate, butyl benzyl adipate, benzyl octyl adipate, tricresyl phosphate, trioctyl phosphate and mixtures thereof; straight or branched chain hydrocarbons, including those straight or branched chain hydrocarbons having a boiling point of greater than about 80° C.; partially hydrogenated terphenyls, dialkyl phthalates, alkyl biphenyls, including monoisopropylbiphenyl, alkylated naphthalene, including dipropylnaphthalene, petroleum spirits, including kerosene, mineral oil and mixtures thereof; aromatic solvents, including benzene, toluene and mixtures thereof; silicone oils; and mixtures thereof.

In one aspect, said encapsulates' wall material may comprise a suitable resin including the reaction product of an aldehyde and an amine, suitable aldehydes include, formaldehyde.

Suitable amines include melamine, urea, benzoguanamine, glycoluril, and mixtures thereof.

Suitable melamines include, methylol melamine, methylated methylol melamine, imino melamine and mixtures thereof. Suitable ureas include, dimethylol urea, methylated dimethylol urea, urea-resorcinol, and mixtures thereof.

In one aspect, suitable formaldehyde scavengers may be employed with the encapsulates, for example, in a capsule slurry and/or added to a consumer product before, during or after the encapsulates are added to such consumer product.

Suitable capsules can be purchased from Appleton Papers Inc. of Appleton, Wis. USA.

In addition, the materials for making the aforementioned encapsulates can be obtained from Solutia Inc. (St Louis, Mo. U.S.A.), Cytec Industries (West Paterson, N.J. U.S.A.), sigma-Aldrich (St. Louis, Mo. U.S.A.), CP Kelco Corp. of San Diego, Calif., USA; BASF AG of Ludwigshafen, Germany; Rhodia Corp. of Cranbury, N.J., USA; Hercules Corp. of Wilmington, Del., USA; Agrium Inc. of Calgary, Alberta, Canada, ISP of New Jersey U.S.A., Akzo Nobel of Chicago, Ill., USA; Stroever Shellac Bremen of Bremen, Germany; Dow Chemical Company of Midland, Mich., USA; Bayer AG of Leverkusen, Germany; Sigma-Aldrich Corp., St. Louis, Mo., USA.

The composition may comprise a structurant selected from the group consisting of diglycerides and triglycerides, ethylene glycol distearate microcrystalline cellulose, cellulose-based materials, microfiber cellulose, biopolymers, xanthan gum, gellan gum, and mixtures thereof.

### Polymers

The cleaning composition may comprise one or more polymers. Examples are carboxymethylcellulose, poly(vinyl-pyrrolidone), poly (ethylene glycol), poly(vinyl alcohol), poly(vinylpyridine-N-oxide), poly(vinylimidazole), polycarboxylates such as polyacrylates, maleic/acrylic acid copolymers and lauryl methacrylate/acrylic acid co-polymers and amphiphilic polymers.

The detergent may contain a bleaching system, which may comprise a H₂O₂ source such as perborate or percarbonate which may be combined with a peracid-forming bleach activator such as tetraacetylethylenediamine or nonanoyloxybenzenesulfonate. Alternatively, the bleaching system may comprise peroxyacids of, e.g., the amide, imide, or sulfone type. In general, when a bleaching agent is used, the compositions of the present invention may comprise from about 0.1% to about 50% or even from about 0.1% to about 25% bleaching agent by weight of the subject cleaning composition.

Chelating Agents—The cleaning compositions herein may contain a chelating agent. Suitable chelating agents include copper, iron and/or manganese chelating agents and mixtures thereof. When a chelating agent is used, the subject cleaning composition may comprise from about 0.005% to about 15% or even from about 3.0% to about 10% chelating agent by weight of the subject cleaning composition. Suitable chelants include DTPA (Diethylene triamine pentaacetic acid), HEDP (Hydroxyethane diphosphonic acid), DTPMP (Diethylene triamine penta(methylene phosphonic acid)), 1,2-Dihydroxybenzene-3,5-disulfonic acid disodium salt hydrate, ethylenediamine, diethylene triamine, ethylenediaminedisuccinic acid (EDDS), N-hydroxyethylethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP) and derivatives thereof.

The enzyme variants of the invention may be stabilized using conventional stabilizing agents, and/or protease inhibitors e.g., a polyol such as propylene glycol or glycerol, a sugar or sugar alcohol, salts such as sodium chloride and potassium chloride, lactic acid, formic acid, boric acid, or a boric acid derivative, e.g., an aromatic borate ester, or a phenyl boronic acid derivative such as 4-formylphenyl boronic acid, or a peptide aldehyde such as di-, tri- or tetrapeptide aldehydes or aldehyde analogues (either of the form B1-B0-R wherein, R is H, CH3, CX3, CHX2, or CH2X (X=halogen), B0 is a single amino acid residue (preferably with an optionally substituted aliphatic or aromatic side chain); and 1 consists of one or more amino acid residues (preferably one, two or three), optionally comprising an N-terminal protection group, or as described in WO09118375, WO98/13459) or a protease inhibitor of the protein type such as RASI, BASI, WASI (bifunctional alpha-amylase/subtilisin inhibitors of rice, barley and wheat) or C12 or SSI. In some embodiments, the enzymes employed herein are stabilized by the presence of water-soluble sources of zinc (II), calcium (II) and/or magnesium (II) ions in the finished compositions that provide such ions to the enzymes, as well as other metal ions (e.g., barium (II), scandium (II), iron (II), manganese (II), aluminum (Ill), Tin (II), cobalt (II), copper (II), Nickel (II), and oxovanadium (IV)).

The composition may comprise an enzyme stabilizer selected from the group consisting of (a) inorganic salts selected from the group consisting of calcium salts, magnesium salts and mixtures thereof; (b) carbohydrates selected from the group consisting of oligosaccharides, polysaccharides and mixtures thereof; (c) mass efficient reversible protease inhibitors selected from the group consisting of phenyl boronic acid and derivatives thereof; and (d) mixtures thereof.

The composition may comprise: (1) reversible protease inhibitors such as a boron containing compound; (2) 1-2 propane diol; (3) calcium formate and/or sodium formate; and (4) any combination thereof.

The cleaning composition may also contain (as cleaning adjunct) other conventional detergent ingredients such as e.g. fabric conditioners including clays, foam boosters, suds suppressors, anti-corrosion agents, soil-suspending agents, anti-soil re-deposition agents, dyes, bactericides, optical brighteners, hydrotropes, tarnish inhibitors, organic solvents such as ethanol or perfumes. Furthermore, the detergent could contain a pre-spotter or a booster, which is added to the wash to increase the general cleaning level, some of these additives may also be used as a pre-treatment agent applied to the textile before the washing step.

It is at present contemplated that in the cleaning compositions any enzyme, in particular the enzyme essential to the present invention, may be added in an amount corresponding to 0.001-100 mg of enzyme protein per liter of wash liquor, preferably 0.005-5 mg of enzyme protein per liter of wash liquor, more preferably 0.01-1 mg of enzyme protein per liter of wash liquor and in particular 0.1-1 mg of enzyme protein per liter of wash liquor. However, the compositions of the present invention comprise at least 0.0001 to about 0.1% weight percent of pure enzyme protein, such as from about 0.0001% to about 0.01%, from about 0.001% to about 0.01% or from about 0.001% to about 0.01%. However, when using a formulated enzyme the detergent composition comprises from about 0.02% to about 20% weight percent, such as or from about 0.05% to about 15% weight, or from about 0.05 to about 20%, or from about 0.05% to about 5%, or from about 0.05% to about 3%. The alpha-amylase variants useful in the present invention may additionally be incorporated in the detergent formulations disclosed in WO 97/07202, which is hereby incorporated as reference.

### Method of Use

The present invention includes a method for cleaning and/or treating a surface, preferably a fabric. In one aspect, such method comprises the steps of optionally washing and/or rinsing said surface, contacting said surface with an aqueous wash liquor comprising the cleaning composition described herein, then optionally washing and/or rinsing said surface.

The aqueous wash liquor preferably has a pH of from about 4 or from about 7 or 8 to about 12, preferably to about 10.5. The compositions are preferably employed at concentrations of from about 500 ppm to about 15,000 ppm in solution. The water temperatures typically range from about 5° C. to about 90° C. The water to fabric ratio is typically from about 1:1 to about 30:1.

The present invention also provides use of a cleaning composition of the invention to provide an improved wash performance in detergent compositions and in detergent applications, such as dish wash or laundering at low temperatures.

In a further aspect, the invention relates to a method for removing a stain from a surface comprising contacting the surface with an hybrid polypeptide or amylases variant as described herein, and a cleaning adjunct, wherein the cleaning adjunct comprises surfactant and optionally one or more cleaning adjunct selected from the list comprising of hydrotropes, builders and co-builders, bleaching systems, polymers, fabric hueing agents and adjunct materials, or any mixture thereof in detergent compositions and in detergent applications. A further aspect is a method for removing a stain from a surface comprising contacting the surface with a cleaning composition as described herein.

### Uses

The present invention is directed to methods for using the polypeptides having alpha-amylase activity, or compositions thereof, in a cleaning process such as laundry or hard surface cleaning processes including automated dish wash.

The soils and stains that are important for cleaning are composed of many different substances, and a range of different enzymes, all with different substrate specificities, have been developed for use in detergents both in relation to laundry and hard surface cleaning, such as dishwashing. These enzymes are considered to provide an enzyme detergency benefit, since they specifically improve stain removal in the cleaning process that they are used in, compared to the same process without enzymes. Stain removing enzymes that are known in the art include enzymes such as proteases, amylases, lipases, cutinases, cellulases, endoglucanases, xyloglucanases, pectinases, pectin lyases, xanthanases, peroxidaes, lichenases, haloperoxygenases, catalases and mannanases.

In one aspect, the invention concerns the use of alpha-amylases of the present invention in detergent compositions, for use in cleaning hard-surfaces, such as dish wash, or in laundering or for stain removal. In an additional aspect, the present invention demonstrates that the use of the alpha amylases of the invention have an improved wash performance in detergent compositions and in detergent applications, such as dish wash or laundering at low temperatures.

Another aspect of the invention is the use of the detergent composition comprising an alpha-amylase of the present invention together with one or more surfactants and optionally one or more detergent components, selected from the list comprising of hydrotropes, builders and co-builders, bleaching systems, polymers, fabric hueing agents and adjunct materials, or any mixture thereof in detergent compositions and in detergent applications.

A further aspect is the use of the detergent composition comprising an alpha-amylase of the present invention together with one or more surfactants, and one or more additional enzymes selected from the group comprising of proteases, lipases, cutinases, cellulases, endoglucanases, xyloglucanases, pectinases, pectin lyases, xanthanases, peroxidaes, lichenases, haloperoxygenases, catalases and mannanases, or any mixture thereof in detergent compositions and in detergent applications.

In another aspect, the invention relates to a dish wash process which may be for household dish wash as well as industrial dish wash. The term “dish wash” as used herein, refers to both manual dish wash and automated dish wash. Furthermore, the invention relates to a process for the washing of hard surfaces (e.g. cutlery such as knives, forks, spoons; crockery such as plates, glasses, bowls; and pans) where the process comprises treating the hard surface with a washing solution containing a detergent composition and an alpha-amylase variant of the present invention. The hard surface washing can for example be carried out using a household or an industrial dishwasher or be carried out by hand using a detergent composition containing an alpha-amylase of the invention, optionally together with one or more further enzymes selected from the group comprising of proteases, amylases, lipases, cutinases, cellulases, endoglucanases, xyloglucanases, pectinases, pectin lyases, xanthanases, peroxidaes, haloperoxygenases, catalases, mannanases, or any mixture thereof.

In a further aspect, the invention relates to a method for removing a stain from a surface comprising contacting the surface with a composition comprising an alpha-amylase of the present invention together with one or more surfactants and optionally one or more detergent components selected from the list comprising of hydrotropes, builders and co-builders, bleaching systems, polymers, fabric hueing agents and adjunct materials, or any mixture thereof in detergent compositions and in detergent applications. A further aspect is a method for removing a stain from a surface comprising contacting the surface with a composition comprising an alpha-amylase variant of the present invention together with one or more surfactants, one or more additional enzymes selected from the group comprising of proteases, lipases, cutinases, cellulases, endoglucanases, xyloglucanases, pectinases, pectin lyases, xanthanases, peroxidaes, lichenases, haloperoxygenases, catalases and mannanases, or any mixture thereof in detergent compositions and in detergent applications.

### Method of Use

The present invention also relates to a method for cleaning and/or treating a situs inter alia a surface or fabric. In one aspect, such method comprises the steps of optionally washing and/or rinsing said surface or fabric, contacting said surface or fabric with any consumer product disclosed in this specification then optionally washing and/or rinsing said surface or fabric is disclosed.

As used herein, washing includes but is not limited to, scrubbing, and mechanical agitation. Drying of such surfaces or fabrics may be accomplished by any one of the common means employed either in domestic or industrial settings. Such means include but are not limited to forced air or still air drying at ambient or elevated temperatures at pressures between 5 and 0.01 atmospheres in the presence or absence of electromagnetic radiation, including sunlight, infrared, ultraviolet and microwave irradiation. In one aspect, said drying may be accomplished at temperatures above ambient by employing an iron wherein, for example, said fabric may be in direct contact with said iron for relatively short or even extended periods of time and wherein pressure may be exerted beyond that otherwise normally present due to gravitational force. In another aspect, said drying may be accomplished at temperatures above ambient by employing a dryer. Apparatus for drying fabric is well known and it is frequently referred to as a clothes dryer. In addition to clothes such appliances are used to dry many other items including towels, sheets, pillowcases, diapers and so forth and such equipment has been accepted as a standard convenience in many nations of the world substantially replacing the use of clothes lines for drying of fabric. Most dryers in use today use heated air which is passed over and or through the fabric as it is tumbled within the dryer. The air may be heated, for example, either electronically, via gas flame, or even with microwave radiation. Such air may be heated from about 15° C. to about 400° C., from about 25° C. to about 200° C., from about 35° C. to about 100° C., or even from about 40° C. to about 85° C. and used in the dryer to dry a surface and/or a fabric. As will be appreciated by one skilled in the art, the cleaning compositions of the present invention are ideally suited for use in laundry applications. Accordingly, the present invention includes a method for laundering a fabric. The method comprises the steps of contacting a fabric to be laundered with a said cleaning laundry solution comprising at least one embodiment of Applicants' cleaning composition, cleaning additive or mixture thereof. The fabric may comprise most any fabric capable of being laundered in normal consumer or institutional use conditions. The solution preferably has a pH of from about 4.0 to about 12.0. The compositions may be employed at concentrations of from about 500 ppm to about 15,000 ppm in solution. The water temperatures typically range from about 5° C. to about 90° C. The water to fabric ratio is typically from about 1:1 to about 30:1.

The invention described above is further defined in the following paragraphs:

Paragraph 1. An alpha-amylase variant of a parent alpha-amylase comprising a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering.
Paragraph 2. The alpha-amylase variant according to paragraph 1, wherein said variant comprises a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the to the SEQ ID No: 1, 2, 3, 4, 5, 6, 7, 8, 9, 10, 11, 12, 13 or 14.
Paragraph 3. The alpha-amylase variant according to any one of the preceding paragraphs, wherein said variant comprises a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the to the SEQ ID No: 1, 2, 3, 4, 5, 6, 7, 8, 9, 10, 11, 12, 13 or 14 and wherein said variant has an improved performance, such as an improved wash performance for a measure of specific activity of Improvement Factor (IF)>1.0, when compared to said parent polypeptide, preferably SEQ ID NO: 2 or 3.
Paragraph 4. The alpha-amylase variant according to any one of preceding paragraphs, wherein said modification in one or more positions corresponding to position: H1, H2, N3, G7, R26, A31, R37, T40, A41, N54, V56, A60, L63, K72, G73, K93, N94, Q98, V103, V104, M105, N106, A109, G110, A111, G113, T114, F116, V117, D118, D123, N126, N128, T131, S132, T134, Y135, Q136, 1137, Q138, W140, K142, D144, P146, T151, Y152, F155, W159, Y160, T165, W167, E169, K172, L173, N174, I176, Y177, K178, A185, W188, E189, E193, N194, N196, F202, A203, L205, T224, N225, Y242, S243, F244, V252, T256, K258, E259, M260, F261, F266, N276, N279, K280, W283, S286, S302, G303, T315, H320, P321, M322, H323, W346, L354, E359, Q360, G361, Y362, V365, Y371, A383, E390, N394, F395, D405, I409, R414, H420, N422, T427, G455, K457, P458, N470, S472, V473, N474, G475, G476, using SEQ ID NO: 1 for numbering.
Paragraph 5. The alpha-amylase variant according to any one of preceeding paragraphs, wherein wherein said modification in one or more positions corresponding to positions: H1*, H2, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering.
Paragraph 6. The alpha-amylase variant according to any one of preceding paragraphs, wherein the variant comprises deletion at two or more positions corresponding to positions R180, S181, T182, and G183, using SEQ ID NO: 1 for numbering.
Paragraph 7. The alpha-amylase variant according to paragraph 6, wherein the deletion is selected from the group consisting of R180*+S181*, R180*+T182*, R180*+G183*, S181*+T182*, S181*+G183*, or T182*+G183*, using SEQ ID NO: 1 for numbering.
Paragraph 8. The alpha-amylase variant according to any one of preceding paragraphs, wherein the variant comprises of one or more a modification selected from the group consisting of: H1*+G7A+R180*+S181*, H1*+G7A+R180*+S181*+Y242F, H1*+G7A+R180*+S181*+Y242F+N279S, H1*+G7A+R180*+S181*+Y242F+N279S+G303R, H1*+G7A+R180*+S181*+Y242F+E259G+N279S+G303R, H1*+G7A+R180*+S181*+Y242F+E259G+N279S+G303R+E390A, H1*+G7A+R180*+S181*+Y242F+E259G+N279S+G303R+E390A+G475K, H1*+G7A+R180*+S181*+Y242F+E259G+N279S+E390A+G476A, H1*+G7A+R180*+S181*+Y242F+E259G+N279S+G303R+E390A+G476A, H1*+G7A+R180*+S181*+D118T+Y242F+E259G+N279S+G303R+E390A+G475K, H1*+G7A+R180*+S181*+D118T+W140Y+Y242F+E259G+N279S+E390A+G476A, H1*+G7A+R180*+S181*+D118T+W140Y+Y242F+E259G+N279S+G303R+E390A+G476A, H1*+G7A+R180*+S181*+D118T+W140Y+Y242F+E259G+N279S+G303R+E390A+G475K, H1*+G7A+R180*+S181*+D118T+W140Y+Y242F+E259G+N279S+E390A+G476A, H1*+G7A+R180*+S181*+D118T+W140Y+Y242F+E259G+N279S+G303R+E390A+G476A, H1*+G7A+R180*+S181*+D118T+W140Y+N194F+Y242F+E259G+N279S+G303R+E390A+G475K, H1*+G7A+R180*+S181*+D118T+W140Y+N194F+Y242F+E259G+N279S+E390A+G476A, H1*+G7A+R180*+S181*+D118T+W140Y+N194F+Y242F+E259G+N279S+G303R+E390A+G476A, H1*+G7A+R180*+S181*+D118T+W140Y+N194F+Y242F+E259G+K280Y+N279S+G303R+E390A+G475K, H1*+G7A+R180*+S181*+D118T+W140Y+N194F+Y242F+E259G+K280Y+N279S+E390A+G476A, H1*+G7A+R180*+S181*+D118T+W140Y+N194F+Y242F+E259G+K280Y+N279S+G303R+E390A+G476A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13, or 14 and wherein the variant has alpha-amylase activity.
Paragraph 9. The alpha-amylase variant according to any one of preceding paragraphs, wherein the variant comprises or consists of one or more a modification selected from the group consisting of: H1*+G7A+R180*+S181*+Y242F+E259G+N279S+G303R+E390A+G475K, H1*+G7A+S181*+T182*+Y242F+E259G+N279S+G303R+E390A+G475K, H1*+G7A+R180*+S181*+T182S+Y242F+E259G+N279S+G303R+E390A+G475K, H1*+G7A+R180*+T182L+G183*+Y242F+E259G+N279S+G303R+E390A+G475K, H1*+G7A+S181 L+T182*+G183*+Y242F+E259G+N279S+G303R+E390A+G475K, H1*+G7A+R180S+T182*+G183*+Y242F+E259G+N279S+G303R+E390A+G475K, H1*+G7A+A60P+R180*+S181*+Y242F+E259G+N279S+G303R+E390A+G475K, H1*+G7A+M105L+R180*+S181*+Y242F+E259G+N279S+G303R+E390A+G475K, H1*+G7A+M105+R180*+S181*+Y242F+E259G+N279S+G303R+E390A+G475K, H1*+G7A+R180*+S181*+T182D+Y242F+E259G+N279S+G303R+E390A+G475K, H1*+G7A+R180*+S181*+T182E+Y242F+E259G+N279S+G303R+E390A+G475K, H1*+G7A+R180*+S181*+T182G+Y242F+E259G+N279S+G303R+E390A+G475K, H1*+G7A+R180*+S181*+T182M+Y242F+E259G+N279S+G303R+E390A+G475K, H1*+G7A+R180*+S181*+A185K+Y242F+E259G+N279S+G303R+E390A+G475K, H1*+G7A+R180*+S181*+A185D+Y242F+E259G+N279S+G303R+E390A+G475K, H1*+G7A+R180*+S181*+A185Q+Y242F+E259G+N279S+G303R+E390A+G475K, H1*+G7A+R180*+S181*+A185N+Y242F+E259G+N279S+G303R+E390A+G475K, H1*+G7A+R180*+S181*+A185R+Y242F+E259G+N279S+G303R+E390A+G475K, H1*+G7A+R180*+S181*+A185E+Y242F+E259G+N279S+G303R+E390A+G475K, H1*+G7A+R180*+S181*+E189P+Y242F+E259G+N279S+G303R+E390A+G475K, H1*+G7A+R180*+S181*+L205Y+Y242F+E259G+N279S+G303R+E390A+G475K, H1*+G7A+R180*+S181*+L205F+Y242F+E259G+N279S+G303R+E390A+G475K, H1*+G7A+R180*+S181*+Y242F+S243Q+E259G+N279S+G303R+E390A+G475K, H1*+G7A+R180*+S181*+Y242F+F244L+E259G+N279S+G303R+E390A+G475K, H1*+G7A+R180*+S181*+Y242F+F244I+E259G+N279S+G303R+E390A+G475K, H1*+G7A+R180*+S181*+Y242F+E259G+F266Y+N279S+G303R+E390A+G475K, H1*+G7A+R180*+S181*+N194F+Y242F+E259G+N279S+G303R+E390A+G475K, H1*+G7A+R180*+S181*+N196F+Y242F+E259G+N279S+G303R+E390A+G475K, H1*+G7A+R180*+S181*+N194F+L205Y+Y242F+E259G+N279S+G303R+E390A+G475K, H1*+G7A+R180*+S181*+N194F+L205F+Y242F+E259G+N279S+G303R+E390A+G475K, H1*+G7A+R180*+S181*+N196F+L205Y+Y242F+E259G+N279S+G303R+E390A+G475K, H1*+G7A+R180*+S181*+E189P+Y242F+E259G+N276F+N279S+G303R+E390A+G475K, H1*+G7A+R180*+S181*+E189P+Y242F+E259G+N276Y+N279S+G303R+E390A+G475K, H1*+G7A+R180*+S181*+E189P+Y242F+E259G+N276W+N279S+G303R+E390A+G475K, H1*+G7A+R180*+S181*+E189P+Y242F+E259G+N279S+K280Y+G303R+E390A+G475K, H1*+G7A+R180*+S181*+E189P+Y242F+E259G+N279S+K280W+G303R+E390A+G475K, H1*+G7A+R180*+S181*+E189P+L205F+Y242F+E259G+N276F+N279S+G303R+E390A+G475K, H1*+G7A+R180*+S181*+E189P+L205F+Y242F+E259G+N276Y+N279S+G303R+E390A+G475K, H1*+G7A+R180*+S181*+E189P+L205F+Y242F+E259G+N276W+N279S+G303R+E390A+G475K, H1*+G7A+R180*+S181*+E189P+L205F+Y242F+E259G+N279S+K280F+G303R+E390A+G475K, H1*+G7A+R180*+S181*+E189P+L205F+Y242F+E259G+N279S+K280Y+G303R+E390A+G475K, H1*+G7A+R180*+S181*+E189P+L205F+Y242F+E259G+N279S+K280W+G303R+E390A+G475K, H1*+G7A+R180*+S181*+E189P+L205Y+Y242F+E259G+N276F+N279S+G303R+E390A+G475K, H1*+G7A+R180*+S181*+E189P+L205Y+Y242F+E259G+N276Y+N279S+G303R+E390A+G475K, H1*+G7A+R180*+S181*+E189P+L205Y+Y242F+E259G+N276W+N279S+G303R+E390A+G475K, H1*+G7A+R180*+S181*+E189P+L205Y+Y242F+E259G+N279S+K280F+G303R+E390A+G475K, H1*+G7A+R180*+S181*+E189P+L205Y+Y242F+E259G+N279S+K280Y+G303R+E390A+G475K, H1*+G7A+R180*+S181*+E189P+L205Y+Y242F+E259G+N279S+K280W+G303R+E390A+G475K, H1*+G7A+G113S+F116W+D123N+S181*+T182*+Y242F+E259G+N279S+G303R+E390A+G475K, H1*+G7A+F116L+D123N+N126D+T131 I+S181*+T182*+F202Y+Y242F+E259G+N279S+G303R+E390A+G475K, H1*+G7S+T40N+Q98L+R180*+S181*+Y242F+E259G+N279S+G303R+Y362V+E390A+G475K, H1*+G7A+R180*+S181*+Y242F+E259G+N279S+G303R+M322G+E359W+Q360W+E390A+G475K, H1*+G7A+R180*+S181*+Y242F+E259G+N279S+G303R+M322G+Q360W+E390A+G475K, H1*+G7A+R180*+S181*+Y242F+E259G+N279S+G303R+E359W+Q360W+E390A+G475K, H1*+G7S+R180*+S181*+Y242F+E259G+F261S+N279S+G303R+Y362V+E390A+G475K, H1*+G7A+T40N+Q98L+R180*+S181*+Y242F+E259G+N279S+G303R+E390A+G475K, H1*+G7S+T40N+Q98L+R180*+S181*+Y242F+E259G+N279S+G303R+E390A+G475K, H1*+G7A+R180*+S181*+Y242F+E259G+F261S+N279S+S286K+G303R+Y362V+E390A+G475K, H1*+G7A+R180*+S181*+Y242F+E259G+N279S+W283N+S286K+G303R+H320T+H323A+E390A+G475K, H1*+G7A+R180*+S181*+Y242F+E259G+N279S+G303R+P321H+M322Y+Q360Y+E390A+G475K, H1*+G7A+D123N+I176V+R180*+S181*+Y242F+E259G+N279S+G303R+E390A+G475K, H1*+G7A+G113A+F116W+D123N+T131V+Q136K+E169Q+K172Q+R180*+S181*+Y242F+E259G+N279S+G303R+E390A+G475K, H1*+G7A+G113S+F116W+D123N+R180*+S181*+Y242F+E259G+N279S+G303R+E390A+G475K, H1*+G7A+F116L+D123N+N126D+T131 I+R180*+S181*+F202Y+Y242F+E259G+N279S+G303R+E390A+G475K, H1*+G7A+N106D+A109G+F116W+D123N+N126D+T131I+T165V+N174S+R180*+S181*+T182I+F202Y+Y242F+E259G+N279S+G303R+E390A+G475K, H1*+G7S+T40N+Q98L+R180*+S181*+Y242F+E259G+N279S+G303R+H320T+E390A+G475K, H1*+G7S+T40N+Q98L+R180*+S181*+Y242F+E259G+N279S+G303R+Y362V+E390A+G475K, H1*+G7A+R180*+S181*+Y242F+E259G+N279S+G303R+M322G+E390A+G475K, H1*+G7A+R180*+S181*+Y242F+E259G+N279S+G303R+M322G+Q360W+E390A+G475K, H1*+G7A+T40N+R180*+S181*+Y242F+E259G+N279S+G303R+E390A+G475K, H1*+G7S+Q98L+R180*+S181*+Y242F+E259G+N279S+G303R+E390A+G475K, H1*+G7S+R180*+S181*+Y242F+E259G+F261K+N279S+G303R+E390A+G475K, H1*+G7S+R180*+S181*+Y242F+E259G+N279S+G303R+Y362V+E390A+G475K, H1*+G7A+T40N+Q98L+R180*+S181*+Y242F+E259G+N279S+G303R+E390A+G475K, H1*+G7A+R180*+S181*+Y242F+E259G+N279S+G303R+Y362V+E390A+G475K, H1*+G7S+R180*+S181*+Y242F+E259G+F261S+N279S+G303R+E390A+G475K, H1*+G7A+I137F+R180*+S181*+Y242F+E259G+F261S+N279S+G303R+Y362V+E390A+G475K, H1*+G7A+R180*+S181*+Y242F+E259G+N279S+S286K+G303R+H323A+E390A+G475K, H1*+G7A+R180*+S181*+Y242F+E259G+N279S+G303R+H320T+H323A+E390A+G475K, H1*+G7A+R180*+S181*+Y242F+E259G+N279S+G303R+P321H+M322Y+E390A+G475K, H1*+G7A+R180*+S181*+Y242F+E259G+N279S+G303R+P321H+Q360Y+E390A+G475K, H1*+G7A+R180*+S181*+Y242F+E259G+N279S+G303R+M322Y+Q360Y+E390A+G475K, H1*+G7S+T40N+R180*+S181*+Y242F+E259G+N279S+G303R+Y362V+E390A+G475K, H1*+G7S+R180*+S181*+Y242F+E259G+F261S+N279S+G303R+Y362V+E390A+G475K, H1*+G7A+R180*+S181*+Y242F+E259G+F261S+N279S+S286K+G303R+Y362V+E390A+G475K, H1*+G7A+R180*+S181*+Y242F+E259G+N279S+S286L+G303R+E390A+G475K, H1*+G7A+G110K+R180*+S181*+Y242F+E259G+N279S+G303R+E390A+G475K, H1*+G7A+W140S+R180*+S181*+Y242F+E259G+N279S+G303R+E390A+G475K, H1*+G7A+W167P+R180*+S181*+Y242F+E259G+N279S+G303R+E390A+G475K, H1*+G7A+R180*+S181*+Y242F+E259G+N279S+G303R+H323A+E390A+G475K, H1*+G7A+R180*+S181*+Y242F+E259G+N279S+W283N+G303R+H320T+H323A+E390A+G475K, H1*+G7A+R180*+S181*+Y242F+E259G+N279S+G303R+H320T+E390A+G475K, H1*+G7A+R180*+S181*+Y242F+E259G+N279S+G303R+P321H+E390A+G475K, H1*+G7A+R180*+S181*+Y242F+E259G+N279S+G303R+M322Y+E390A+G475K, H1*+G7A+R180*+S181*+Y242F+E259G+N279S+G303R+Q360Y+E390A+G475K, H1*+G7S+T40N+Q98L+R180*+S181*+Y242F+E259G+N279S+G303R+E390A+G475K, H1*+G7A+Q98L+R180*+S181*+Y242F+E259G+F261K+N279S+G303R+Y362V+E390A+G475K, H1*+G7A+Q98L+R180*+S181*+Y242F+E259G+N279S+G303R+E390A+G475K, H1*+G7A+R180*+S181*+Y242F+E259G+F261S+N279S+G303R+Y362V+E390A+G475K, H1*+G7A+R180*+S181*+Y242F+E259G+N279S+S286L+G303R+H320T+E390A+G475K, H1*+G7A+R180*+S181*+Y242F+E259G+F261S+N279S+G303R+E390A+G475K, H1*+G7A+W140Y+R180*+S181*+T182D+N194F+L205Y+Y242F+E259G+N279S+G303R+E390A+G475K, H1*+G7A+W140Y+R180*+S181*+T182D+N194F+L205V+Y242F+E259G+N279S+G303R+E390A+G475K, H1*+G7A+D118T+W140Y+R180*+S181*+T182D+N194F+L205Y+Y242F+E259G+N279S+G303R+E390A+G475K, H1*+G7A+F116N+W140Y+R180*+S181*+T182D+N194F+L205Y+Y242F+E259G+N279S+G303R+E390A+G475K, H1*+G7A+W140Y+R180*+S181*+E189P+N194F+L205Y+Y242F+E259G+N279S+K280Y+G303R+E390A+G475K, H1*+G7A+D118T+W140Y+R180*+S181*+E189P+N194F+L205Y+Y242F+E259G+N279S+K280Y+G303R+E390A+G475K, H1*+G7A+F116N+W140Y+R180*+S181*+E189P+N194F+L205Y+Y242F+E259G+N279S+K280Y+G303R+E390A+G475K, H1*+G7A+W140Y+R180*+S181*+T182D+E189P+N194F+L205Y+Y242F+E259G+N279S+G303R+E390A+G475K, H1*+G7A+T134D+W140Y+R180*+S181*+E189P+N194F+L205V+Y242F+E259G+N279S+G303R+E390A+G475K, H1*+G7A+F116N+W140Y+R180*+S181*+T182D+E189P+N194F+L205Y+Y242F+E259G+N279S+G303R+E390A+G475K, H1*+G7A+W140Y+R180*+S181*+T182S+N194F+L205Y+Y242F+E259G+N279S+G303R+E390A+G475K, H1*+G7A+W140Y+R180*+S181*+T182S+N194F+L205V+Y242F+E259G+N279S+G303R+E390A+G475K, H1*+G7A+D118T+W140Y+R180*+S181*+T182S+N194F+L205Y+Y242F+E259G+N279S+G303R+E390A+G475K, H1*+G7A+F116N+W140Y+R180*+S181*+T182S+N194F+L205Y+Y242F+E259G+N279S+G303R+E390A+G475K, H1*+G7A+W140Y+R180*+S181*+T182S+E189P+N194F+L205Y+Y242F+E259G+N279S+G303R+E390A+G475K, H1*+G7A+W140Y+R180*+S181*+T182S+E189P+N194F+L205V+Y242F+E259G+N279S+G303R+E390A+G475K, H1*+G7A+F116N+W140Y+R180*+S181*+T182S+E189P+N194F+L205Y+Y242F+E259G+N279S+K280Y+G303R+E390A+G475K, H1*+G7A+D123N+W140Y+R180*+S181*+N194F+L205V+Y242F+E259G+N279S+G303R+E390A+G475K, H1*+G7A+D123N+W140Y+I176V+R180*+S181*+N194F+L205V+Y242F+E259G+N279S+G303R+E390A+G475K, H1*+G7A+G113S+F116W+D123N+W140Y+R180*+S181*+N194F+L205V+Y242F+E259G+N279S+G303R+E390A+G475K, H1*+G7A+F116L+D123N+N126D+T131I+W140Y+R180*+S181*+N194F+F202Y+L205V+Y242F+E259G+N279S+G303R+E390A+G475K, H1*+G7A+N106D+A109G+W140Y+R180*+S181*+N194F+F202Y+L205V+Y242F+E259G+N279S+G303R+E390A+G475K, H1*+G7A+D123N+W140Y+R180*+S181*+N194F+L205Y+Y242F+E259G+N279S+G303R+E390A+G475K, H1*+G7A+D123N+W140Y+I176V+R180*+S181*+N194F+L205Y+Y242F+E259G+N279S+G303R+E390A+G475K, H1*+G7A+G113S+F116W+D123N+W140Y+R180*+S181*+N194F+L205Y+Y242F+E259G+N279S+G303R+E390A+G475K, H1*+G7A+F116L+D123N+N126D+T131I+W140Y+R180*+S181*+N194F+F202Y+L205Y+Y242F+E259G+N279S+G303R+E390A+G475K, H1*+G7A+Q98L+G110K+P146S+R180*+S181*+Y242F+E259G+N279S+G303R+A383D+E390A+G475K, H1*+N3S+G7A+Q98L+G110K+R180*+S181*+Y242F+V252+E259G+N279S+G303R+E390A+G475K, H1*+G7H+Q98L+R180*+S181*+Y242F+E259G+N279S+G303R+A383D+E390A+D405G+G475K, H1*+G7H+Q98L+G110K+R180*+S181*+Y242F+E259G+N279S+G303R+E390A+G475K, H1*+G7A+Q98L+G110K+S132D+Y135E+R180*+S181*+Y242F+E259G+N279S+G303R+T315A+E390A+G475K, H1*+G7H+G110K+R180*+S181*+Y242F+E259G+N279S+G303R+E390A+G475K, H1*+A60V+Q98L+W140Y+R180*+S181*+N194F+L205Y+Y242F+E259G+N279S+G303R+A383D+E390A+G475K, H1*+G7A+W140Y+T151A+R180*+S181*+N194F+L205Y+Y242F+K258V+E259V+N279S+G303R+E390A+G475K, H1*+N3S+G7A+N94D+Q98L+W140Y+R180*+S181*+N194F+L205Y+Y242F+V252I+E259G+N279S+G303R+E390A+G475K, H1*+G7A+Q98L+G110K+R180*+S181*+Y242F+T256A+E259G+N279S+G303R+E390A+G475K, H1*+G7A+Q98L+G110K+R180*+S181*+Y242F+E259G+N279S+G303R+A383D+E390A+G475K, H1*+N3S+G7A+N94D+Q98L+G110K+R180*+S181*+Y242F+V252I+E259G+N279S+G303R+E390A+G475K, H1*+G7A+Q98L+G110K+R180*+S181*+Y242F+E259G+N279S+G303R+E390A+G475K, H1*+G7H+Q98L+G110K+R180*+S181*+Y242F+E259G+N279S+G303R+E390A+G475K, H1*+G7A+W140Y+T151A+R180*+S181*+N194F+L205Y+Y242F+K258V+E259V+N279S+G303R+E390A+G475K, H1*+G7A+Q98L+G110K+R180*+S181*+Y242F+E259G+N279S+G303R+E390A+G475K, H1*+G7A+R180*+S181*+Y242F+E259G+N279S+S286L+G303R+Y362V+E390A+G475K, H1*+G7A+R180*+S181*+Y242F+E259G+N279S+W283N+S286K+G303R+E390A+G475K, H1*+G7A+R180*+S181*+Y242F+E259G+N279S+W283N+G303R+H320T+E390A+G475K, H1*+G7A+R180*+S181*+Y242F+E259G+N279S+W283N+G303R+H323A+E390A+G475K, H1*+G7A+R180*+S181*+Y242F+E259G+N279S+S286K+G303R+H320T+E390A+G475K, H1*+G7A+R180*+S181*+Y242F+E259G+N279S+S286K+G303R+Y362V+E390A+G475K, H1*+G7S+R180*+S181*+Y242F+E259G+N279S+G303R+E390A+G475K, H1*+G7A+Q98L+R180*+S181*+Y242F+E259G+N279S+G303R+E390A+G475K, H1*+G7A+R180*+S181*+Y242F+E259G+F261K+N279S+G303R+E390A+G475K, H1*+G7A+R180*+S181*+Y242F+E259G+N279S+G303R+M322G+E359W+E390A+G475K, H1*+G7A+F116N+W140Y+R180*+S181*+T182D+E189P+N194F+L205Y+Y242F+E259G+N279S+K280Y+G303R+E390A+G475K, H1*+G7A+F116N+W140Y+R180*+S181*+T182D+E189P+N194F+Y242F+E259G+N279S+K280Y+G303R+E390A+G475K, H1*+G7A+F116N+W140Y+R180*+S181*+T182S+E189P+N194F+Y242F+E259G+N279S+K280Y+G303R+E390A+G475K, H1*+G7A+F116N+W140Y+R180*+S181*+T182D+N194F+L205Y+Y242F+E259G+F266Y+N279S+G303R+E390A+G475K, H1*+G7A+F116N+W140Y+R180*+S181*+A185Q+N194F+L205Y+Y242F+E259G+F266Y+N279S+G303R+E390A+G475K, H1*+G7A+F116N+W140Y+R180*+S181*+T182D+A185Q+N194F+L205Y+Y242F+E259G+F266Y+N279S+G303R+E390A+G475K, H1*+G7A+F116N+W140Y+R180*+S181*+N194F+L205Y+Y242F+E259G+F266Y+N279S+G303R+E390A+G475K, H1*+G7A+F116N+W140Y+R180*+S181*+A185Q+N194F+L205Y+Y242F+E259G+N279S+G303R+E390A+G475K, H1*+G7A+F116N+W140Y+R180*+S181*+T182D+A185Q+N194F+L205Y+Y242F+E259G+N279S+G303R+E390A+G475K, H1*+G7A+D118T+W140Y+R180*+S181*+E189P+N194F+Y242F+E259G+N279S+K280Y+G303R+E390A+G475K, H1*+G7A+D118T+W140Y+R180*+S181*+T182S+E189P+N194F+L205Y+Y242F+E259G+N279S+K280Y+G303R+E390A+G475K, H1*+G7A+D118T+W140Y+R180*+S181*+T182D+N194F+L205Y+Y242F+E259G+F266Y+N279S+G303R+E390A+G475K, H1*+G7A+D118T+W140Y+R180*+S181*+A185Q+N194F+L205Y+Y242F+E259G+F266Y+N279S+G303R+E390A+G475K, H1*+G7A+D118T+W140Y+R180*+S181*+T182D+A185Q+N194F+L205Y+Y242F+E259G+F266Y+N279S+G303R+E390A+G475K, H1*+G7A+D118T+W140Y+R180*+S181*+N194F+L205Y+Y242F+E259G+F266Y+N279S+G303R+E390A+G475K, H1*+G7A+D118T+W140Y+R180*+S181*+A185Q+N194F+L205Y+Y242F+E259G+N279S+G303R+E390A+G475K, H1*+G7A+D118T+W140Y+R180*+S181*+T182D+A185Q+N194F+L205Y+Y242F+E259G+N279S+G303R+E390A+G475K, H1*+G7A+W140Y+R180*+S181*+E189P+N194F+L205V+Y242F+E259G+N279S+K280Y+G303R+E390A+G475K, H1*+G7A+W140Y+R180*+S181*+T182D+E189P+N194F+L205V+Y242F+E259G+N279S+K280Y+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+T182S+E189P+N194F+Y242F+E259G+N279S+K280Y+G303R+E390A+G475K, H1*+G7A+W140Y+R180*+S181*+T182D+N194F+L205V+Y242F+E259G+F266Y+N279S+G303R+E390A+G475K, H1*+G7A+W140Y+R180*+S181*+A185Q+N194F+L205V+Y242F+E259G+F266Y+N279S+G303R+E390A+G475K, H1*+G7A+W140Y+R180*+S181*+T182D+A185Q+N194F+L205V+Y242F+E259G+F266Y+N279S+G303R+E390A+G475K, H1*+G7A+W140Y+R180*+S181*+N194F+L205V+Y242F+E259G+F266Y+N279S+G303R+E390A+G475K, H1*+G7A+W140Y+R180*+S181*+A185Q+N194F+L205V+Y242F+E259G+N279S+G303R+E390A+G475K, H1*+G7A+W140Y+R180*+S181*+T182D+A185Q+N194F+L205V+Y242F+E259G+N279S+G303R+E390A+G475K, H1*+G7A+W140Y+R180*+S181*+T182D+E189P+N194F+L205Y+Y242F+E259G+N279S+K280Y+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+T182S+E189P+N194F+L205Y+Y242F+E259G+N279S+K280Y+G303R+E390A+G475K, H1*+G7A+W140Y+R180*+S181*+T182S+E189P+N194F+Y242F+E259G+N279S+K280Y+G303R+E390A+G475K, H1*+G7A+W140Y+R180*+S181*+T182D+N194F+L205Y+Y242F+E259G+F266Y+N279S+G303R+E390A+G475K, H1*+G7A+W140Y+R180*+S181*+A185Q+N194F+L205Y+Y242F+E259G+F266Y+N279S+G303R+E390A+G475K, H1*+G7A+W140Y+R180*+S181*+T182D+A185Q+N194F+L205Y+Y242F+E259G+F266Y+N279S+G303R+E390A+G475K, H1*+G7A+W140Y+R180*+S181*+A185Q+N194F+L205Y+Y242F+E259G+N279S+G303R+E390A+G475K, H1*+G7A+W140Y+R180*+S181*+T182D+A185Q+N194F+L205Y+Y242F+E259G+N279S+G303R+E390A+G475K, H1*+G7A+F116N+W140Y+R180*+S181*+E189P+N194F+Y242F+E259G+N279S+K280Y+G303R+E390A+G475K, H1*+G7A+D118T+W140Y+R180*+S181*+T182D+E189P+N194F+L205Y+Y242F+E259G+N279S+K280Y+G303R+E390A+G475K, H1*+G7A+D118T+W140Y+R180*+S181*+T182D+E189P+N194F+Y242F+E259G+N279S+K280Y+G303R+E390A+G475K, H1*+G7A+D118T+W140Y+R180*+S181*+T182S+E189P+N194F+Y242F+E259G+N279S+K280Y+G303R+E390A+G475K, H1*+G7A+W140Y+R180*+S181*+E189P+N194F+Y242F+E259G+N279S+K280Y+G303R+E390A+G475K, H1*+G7A+W140Y+R180*+S181*+E189P+N194F+Y242F+E259G+N279S+K280Y+G303R+E390A+G475K, H1*+G7A+W140Y+R180*+S181*+N194F+L205Y+Y242F+E259G+F266Y+N279S+G303R+E390A+G475K, H1*+G7A+W140Y+R180*+S181*+N194F+L205Y+Y242F+E259G+N279S+S302T+G303R+E390A+G475K, H1*+G7A+W140Y+R180*+S181*+N194F+L205Y+Y242F+E259G+N279S+G303R+E390A+N474F+G475K, H1*+G7A+W140Y+R180*+S181*+N194F+L205Y+Y242F+E259G+N279S+G303R+E390A+N422V+G475K, H1*+G7A+W140Y+R180*+S181*+N194F+L205Y+Y242F+E259G+N279S+G303R+E390A+G476A, H1*+G7A+W140Y+R180*+S181*+N194F+L205Y+Y242F+E259G+N279S+G303R+N394W+G475K, H1*+G7A+W140Y+R180*+S181*+N194F+L205Y+Y242F+E259G+N279S+G303R+E390A+N474L, H1*+G7A+W140Y+R180*+S181*+N194F+L205Y+Y242F+E259G+N279S+S302T+E390A+G475K, H1*+G7A+W140Y+R180*+S181*+N194F+L205Y+Y242F+E259G+N279S+G303R+E390A+G475E, H1*+G7A+W140Y+R180*+S181*+N194F+L205Y+Y242F+E259G+F261P+N279S+G303R+E390A+G475K, H1*+G7A+W140Y+R180*+S181*+N194F+L205Y+Y242F+E259G+N279S+G303R+E390A+N474F, H1*+G7A+W140Y+R180*+S181*+N194F+L205Y+Y242F+E259G+N279S+S302Y+E390A+G475K, H1*+G7A+W140Y+R180*+S181*+N194F+L205Y+Y242F+E259G+N279S+G303R+E390A+N470A+G475K, H1*+G7A+W140Y+R180*+S181*+N194F+L205Y+Y242F+T256D+N279S+G303R+E390A+G475K, H1*+G7A+W140Y+R180*+S181*+N194F+L205Y+Y242L+E259G+N279S+G303R+W346N+E390A+G475K, H1*+G7A+W140Y+R180*+S181*+N194F+L205Y+Y242F+E259G+N279S+G303R+E390A+H420P+G475K, H1*+G7A+R26Y+W140Y+R180*+S181*+N194F+L205Y+Y242F+E259G+N279S+G303R+E390A+G475K, H1*+G7A+R37A+W140Y+R180*+S181*+N194F+L205Y+Y242F+E259G+N279S+G303R+G475K, H1*+W140Y+R180*+S181*+N194F+L205Y+Y242F+E259G+N279S+G303R+Q360Y+E390A+G475K, H1*+G7A+R37N+W140Y+R180*+S181*+N194F+L205Y+Y242F+E259G+N279S+G303R+G475K, H1*+W140Y+R180*+S181*+N194F+L205Y+Y242F+E259G+N279S+G303R+E390A+R414M+G475K, H1*+W140Y+R180*+S181*+N194F+L205Y+Y242F+E259G+F261P+N279S+G303R+E390A+G475K, H1*+W140Y+R180*+S181*+N194F+L205Y+Y242F+E259G+N279S+G303R+H323W+E390A+G475K, H1*+W140Y+R180*+S181*+N194F+L205Y+Y242F+E259G+N279S+G303R+P321Y+E390A+G475K, H1*+W140Y+R180*+S181*+N194F+L205Y+Y242F+E259G+N279S+G303R+Q360M+E390A+G475K, H1*+G7A+W140Y+R180*+S181*+N194F+L205Y+Y242F+E259G+N279S+G303R+Y371S+E390A+G475K, H1*+G7A+W140Y+R180*+S181*+T182D+A185Q+N194F+Y242F+S243Q+E259G+N279S+K280Y+G303R+E390A+G475K, H1*+G7A+W140Y+R180*+S181*+T182D+Y242F+S243Q+E259G+F266Y+N279S+K280Y+G303R+E390A+G475K, H1*+G7A+W140Y+R180*+S181*+A185Q+N194F+Y242F+S243Q+E259G+F266Y+N279S+K280Y+G303R+E390A+G475K, H1*+G7A+W140Y+R180*+S181*+T182D+A185Q+Y242F+E259G+N279S+K280Y+G303R+E390A+G475K, H1*+G7A+W140Y+R180*+S181*+T182D+E189P+Y242F+S243Q+E259G+N279S+K280Y+G303R+E390A+G475K, H1*+G7A+W140Y+R180*+S181*+A185Q+Y242F+E259G+F266Y+N279S+K280Y+G303R+E390A+T427L+G475K, H1*+G7A+W140Y+R180*+S181*+A185Q+E189P+Y242F+E259G+N279S+K280Y+G303R+E390A+G475K, H1*+G7A+W140Y+R180*+S181*+T182D+A185Q+E189P+N194F+Y242F+S243Q+E259G+N279S+K280Y+G303R+E390A+G475K, H1*+G7A+W140Y+R180*+S181*+T182D+A185Q+E189P+N194F+L205Y+Y242F+S243Q+E259G+N279S+K280Y+G303R+E390A+G475K, H1*+G7A+W140Y+R180*+S181*+T182D+E189P+L205Y+Y242F+E259G+F266Y+N279S+K280Y+G303R+E390A+G475K, H1*+G7A+W140Y+R180*+S181*+A185Q+E189P+N194F+Y242F+E259G+F266Y+N279S+K280Y+G303R+E390A+G475K, H1*+G7A+W140Y+R180*+S181*+A185Q+E189P+N194F+Y242F+S243Q+E259G+F266Y+N279S+K280Y+G303R+E390A+G475K, H1*+G7A+W140Y+R180*+S181*+A185Q+E189P+N194F+L205Y+Y242F+S243Q+E259G+F266Y+N279S+K280Y+G303R+E390A+G475K, H1*+G7A+W140Y+R180*+S181*+T182D+A185Q+E189P+N194F+L205Y+Y242F+E259G+F266Y+N279S+K280Y+G303R+E390A+G475K, H1*+G7A+W140Y+R180*+S181*+T182D+A185Q+E189P+N194F+Y242F+E259G+F266Y+N279S+K280Y+G303R+E390A+G475K, H1*+G7A+W140Y+R180*+S181*+A185Q+Y242F+S243Q+E259G+F266Y+N279S+K280Y+G303R+E390A+G475K, H1*+G7A+W140Y+R180*+S181*+T182D+A185Q+Y242F+S243Q+E259G+F266Y+N279S+G303R+E390A+G475K, H1*+G7A+W140Y+R180*+S181*+Y242F+S243Q+E259G+N279S+K280Y+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+A185Q+N194F+Y242F+S243Q+E259G+N279S+K280Y+G303R+E390A+G475K, H1*+G7A+W140Y+R180*+S181*+T182D+N194F+Y242F+S243Q+E259G+N279S+K280Y+G303R+E390A+G475K, H1*+G7A+W140Y+R180*+S181*+A185Q+E189P+N194F+Y242F+E259G+N279S+K280Y+G303R+E390A+G475K, H1*+G7A+W140Y+R180*+S181*+E189P+Y242F+S243Q+E259G+N279S+K280Y+G303R+E390A+G475K, H1*+G7A+W140Y+R180*+S181*+T182D+Y242F+S243Q+E259G+N279S+K280Y+G303R+E390A+G475K, H1*+G7A+W140Y+R180*+S181*+E189P+L205Y+Y242F+S243Q+E259G+N279S+K280Y+G303R+E390A+G475K, H1*+G7A+W140Y+R180*+S181*+Y242F+E259G+F266Y+N279S+K280Y+G303R+E390A+G475K, H1*+G7A+W140Y+R180*+S181*+T182D+A185Q+Y242F+E259G+N279S+G303R+E390A+G475K, H1*+G7A+W140Y+R180*+S181*+T182D+A185Q+N194F+Y242F+S243Q+E259G+N279S+G303R+E390A+G475K, H1*+G7A+W140Y+R180*+S181*+T182D+N194F+Y242F+S243Q+E259G+F266Y+N279S+G303R+E390A+G475K, H1*+G7A+W140Y+R180*+S181*+T182D+E189P+N194F+Y242F+S243Q+E259G+N279S+K280Y+G303R+E390A+G475K, H1*+G7A+W140Y+R180*+S181*+E189P+N194F+Y242F+E259G+F266Y+N279S+K280Y+G303R+E390A+G475K, H1*+G7A+W140Y+R180*+S181*+A185Q+E189P+N194F+L205Y+Y242F+S243Q+E259G+N279S+K280Y+G303R+E390A+G475K, H1*+G7A+W140Y+R180*+S181*+T182D+A185Q+N194F+Y242F+E259G+F266Y+N279S+G303R+E390A+G475K, H1*+G7A+W140Y+R180*+S181*+A185Q+N194F+Y242F+E259G+F266Y+N279S+K280Y+G303R+E390A+G475K, H1*+G7A+W140Y+R180*+S181*+A185Q+E189P+L205Y+Y242F+E259G+N279S+K280Y+G303R+E390A+G475K, H1*+G7A+W140Y+R180*+S181*+Y242F+S243Q+E259G+F266Y+N279S+K280Y+G303R+E390A+G475K, H1*+G7A+W140Y+R180*+S181*+T182D+E189P+L205Y+Y242F+S243Q+E259G+N279S+K280Y+G303R+E390A+I409L+G475K, H1*+G7A+A41D+W140Y+R180*+S181*+N194F+L205Y+Y242F+E259G+N279S+G303R+E390A+G475K, H1*+G7A+T40G+W140Y+R180*+S181*+N194F+L205Y+Y242F+E259G+N279S+G303R+E390A+G475K, L63F+W140Y+L173F+R180*+S181*+N194F+L205Y+Y242F+E259G+N279S+H323W W140Y+L173F+R180*+S181*+N194F+L205Y+N225S+Y242F+E259G+N279S+G303R+H323A, H1*+N3D+G7A+D118T+W140Y+R180*+S181*+T182S+E189P+N194F+L205Y+Y242F+E259G+N279S+K280Y+S302Y+E390A+N474L, H1*+N3D+G7A+D118T+W140Y+R180*+S181*+T182S+E189P+N194F+L205Y+Y242F+E259G+N279S+K280Y+G303R+Y362V+E390A+H420A+N474L, H1*+N3D+G7A+D118T+W140Y+R180*+S181*+T182S+E189P+N194F+L205Y+Y242F+E259G+N279S+K280Y+S302T+V365I+E390A+N474L, H1*+G7A+D118T+W140Y+R180*+S181*+T182S+E189P+N194F+L205Y+Y242F+E259G+N279S+K280Y+S302Y+E390A+H420A+G475K, H1*+G7A+D118T+W140Y+R180*+S181*+T182S+E189P+N194F+L205Y+Y242F+E259G+N279S+K280Y+S302Y+E390A+H420A+G475K, H1*+N3D+G7A+D118T+W140Y+R180*+S181*+T182S+E189P+N194F+L205Y+Y242F+E259G+N279S+K280Y+S302Y+E390A+N474L, H1*+N3D+G7A+D118T+W140Y+R180*+S181*+T182S+E189P+N194F+L205Y+Y242F+E259G+N279S+K280Y+S302T+E390A+H420A+G475K, H1*+G7A+D118T+W140Y+R180*+S181*+T182S+E189P+N194F+L205Y+Y242F+E259G+N279S+K280Y+S302Y+E390A+H420A+G475K, H1*+N3D+G7A+D118T+W140Y+R180*+S181*+T182S+E189P+N194F+L205Y+Y242F+E259G+N279S+K280Y+S302T+E390A+H420A+G475K, H1*+G7A+D118T+W140Y+R180*+S181*+T182S+E189P+N194F+L205Y+Y242F+E259G+N279S+K280Y+G303R+E390A+H420A+G476A, H1*+N3D+G7A+D118T+W140Y+R180*+S181*+T182S+E189P+N194F+L205Y+Y242F+E259G+N279S+K280Y+S302T+E390A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+G7A+D118T+W140Y+R180*+S181*+T182S+E189P+N194F+L205Y+Y242F+E259G+N279S+K280Y+G303R+E390A+H420A+N474L, H1*+N3D+G7A+R37A+D118T+W140Y+R180*+S181*+T182S+E189P+N194F+L205Y+Y242F+E259G+N279S+K280Y+G303R+N394D+N474F, H1*+G7A+D118T+W140Y+R180*+S181*+T182S+E189P+N194F+L205Y+Y242F+E259G+N279S+K280Y+G303R+N394W+H420P+N474L, H1*+G7A+G73L+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+G303R+E390A+G476A, H1*+N3D+G7A+D118T+W140Y+R180*+S181*+T182S+E189P+N194F+L205Y+Y242F+E259G+N279S+K280Y+S302Y+E390A+H420A+G475K, H1*+N3D+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+N394W+G475K, H1*+N3D+G7A+K93R+D118T+W140Y+R180*+S181*+T182S+E189P+N194F+L205Y+Y242F+E259G+N279S+K280Y+G303R+E390A+H420A+N474L, H1*+G7A+R37N+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+G303R+E390D+H420P+G475K, H1*+G7A+D118T+W140Y+R180*+S181*+T182S+E189P+N194F+L205Y+Y242F+E259G+N279S+K280Y+S302T+N394W+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+D118T+W140Y+R180*+S181*+T182S+E189P+N194F+L205Y+Y242F+E259G+N279S+K280Y+G303R+N394W+H420A+G475K, H1*+G7A+R37N+D118T+W140Y+R180*+S181*+T182S+E189P+N194F+L205Y+Y242F+E259G+N279S+K280Y+G303R+E390A+H420A+G475K, H1*+N3D+G7A+D118T+W140Y+R180*+S181*+T182S+E189P+N194F+L205Y+Y242F+E259G+N279S+K280Y+S302T+E390A+G476H, H1*+G7A+D118T+W140Y+R180*+S181*+T182S+E189P+N194F+L205Y+Y242F+E259G+N279S+K280Y+G303R+N394D+H420A+G475K, H1*+N3D+G7A+D118T+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+N279S+K280Y+S302T+E390A+G476A, H1*+G7A+D118T+W140Y+R180*+S181*+T182S+E189P+N194F+L205Y+Y242F+E259G+N279S+K280Y+G303R+E390A+H420P+G475K, H1*+N3D+G7A+D118T+W140Y+R180*+S181*+T182S+E189P+N194F+L205Y+Y242F+E259G+N279S+K280Y+G303R+E390A+H420A+N474L, N3G+G7A+D118T+W140Y+R180*+S181*+T182S+E189P+N194F+L205Y+Y242F+E259G+N279S+K280Y+G303R+E390A+H420A+N474L, H1*+G7A+R37N+D118T+W140Y+R180*+S181*+T182S+E189P+N194F+L205Y+Y242F+E259G+N279S+K280Y+G303R+N394W+H420A+G475K, H1*+N3D+G7A+R37N+D118T+Q138V+W140Y+R180*+S181*+T182S+E189P+N194F+L205Y+Y242F+E259G+N279S+K280Y+G303R+E390A+G475K, H1*+G7A+D118T+W140Y+R180*+S181*+T182S+E189P+N194F+L205Y+Y242F+E259G+N279S+K280Y+S302Y+E390A+G476H, H1*+G7A+D118T+Y135E+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+G303R+N394W+H420A+G475K, H1*+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+G303R+E390A+H420P+G475K, H1*+G7A+V103I+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+M260D+N279S+K280Y+S302Y+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+G303R+H323W+E390A+N474L, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+P146H+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+F261P+N279S+K280Y+G303R+E390A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+Q98L+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+G303R+E390A+N474L, H1*+G7A+D118T+W140Y+R180*+S181*+T182S+E189P+N194F+L205Y+Y242F+E259G+N279S+K280Y+G303R+P321H+E390A+G475K, H1*+G7A+D118T+W140Y+R180*+S181*+T182S+E189P+N194F+L205Y+Y242F+E259G+N279S+K280Y+G303R+P321H+E390A+N474L, H1*+G7A+D118T+S132D+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+G303R+N394D+R414M+G475K, H1*+G7A+T40G+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+T224F+Y242F+E259G+N279S+K280Y+G303R+N394W+H420A+G475K, H1*+G7A+D118T+W140Y+R180*+S181*+T182S+E189P+N194F+L205Y+Y242F+E259G+M260D+N279S+K280Y+S302T+E390A+G475K, H1*+G7A+D118T+W140Y+R180*+S181*+T182S+E189P+N194F+L205Y+Y242F+E259G+M260W+N279S+K280Y+S302Y+E390A+G476A, H1*+G7A+D118T+Y135E+W140Y+R180*+S181*+T182S+E189P+N194F+L205Y+Y242F+E259G+M260D+N279S+K280Y+G303R+W346N+E390A+G475K, H1*+G7A+D118T+W140Y+R180*+S181*+T182S+E189P+N194F+L205Y+Y242F+E259G+N279S+K280Y+G303R+P321H+Q360Y+E390A+G476A, H1*+G7A+D118T+W140Y+R180*+S181*+T182S+E189P+N194F+L205Y+Y242F+E259G+M260D+N279S+K280Y+S302Y+E390A+N470M+G475K, H1*+G7A+D118T+W140Y+R180*+S181*+T182S+E189P+N194F+L205Y+Y242F+E259G+M260W+N279S+K280Y+G303R+E390A+G476A, H1*+G7A+D118T+W140Y+R180*+S181*+T182S+E189P+N194F+L205Y+T224Y+Y242F+N279S+K280Y+G303R+E390A+G476A, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302Y+E390A+N474F, H1*+G7A+D118T+W140Y+R180*+S181*+T182S+E189P+N194F+L205Y+Y242F+E259G+M260W+N279S+K280Y+G303R+E390A+G476A, H1*+G7A+D118T+W140Y+R180*+S181*+T182S+E189P+N194F+L205Y+Y242F+E259G+M260W+N279S+K280Y+S302Y+E390A+G475K, H1*+N3D+G7A+D118T+W140Y+R180*+S181*+T182S+E189P+N194F+L205Y+Y242F+E259G+N279S+K280Y+S302T+E390A+H420A+G475K, H1*+G7A+D118T+W140Y+R180*+S181*+T182S+E189P+N194F+L205Y+T224Y+Y242F+E259G+N279S+K280Y+G303R+E390A+N474L, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+E390A+H420A+G475K, H1*+G7A+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+P146H+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+G475K, H1*+N3G+G7A+D118T+W140Y+R180*+S181*+T182S+E189P+N194F+L205Y+Y242F+E259G+N279S+K280Y+S302T+E390A+G475K, H1*+G7A+D118T+S132L+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+G475K, H1*+D118T+W140Y+R180*+S181*+T182S+E189P+N194F+L205Y+Y242F+E259G+N279S+K280Y+G303R+E390A+G475K, H1*+G7A+W140Y+R180*+S181*+T182S+E189P+N194F+L205Y+Y242F+E259G+N279S+K280Y+G303R+E390A+G475K, H1*+G7A+D118T+R180*+S181*+T182S+E189P+N194F+L205Y+Y242F+E259G+N279S+K280Y+G303R+E390A+G475K, H1*+G7A+D118T+W140Y+R180*+S181*+E189P+N194F+L205Y+Y242F+E259G+N279S+K280Y+G303R+E390A+G475K, H1*+G7A+D118T+W140Y+R180*+S181*+T182S+N194F+L205Y+Y242F+E259G+N279S+K280Y+G303R+E390A+G475K, H1*+G7A+D118T+W140Y+R180*+S181*+T182S+E189P+L205Y+Y242F+E259G+N279S+K280Y+G303R+E390A+G475K, H1*+G7A+D118T+W140Y+R180*+S181*+T182S+E189P+N194F+Y242F+E259G+N279S+K280Y+G303R+E390A+G475K, H1*+G7A+D118T+W140Y+R180*+S181*+T182S+E189P+N194F+L205Y+E259G+N279S+K280Y+G303R+E390A+G475K, H1*+G7A+D118T+W140Y+R180*+S181*+T182S+E189P+N194F+L205Y+Y242F+N279S+K280Y+G303R+E390A+G475K, H1*+G7A+D118T+W140Y+R180*+S181*+T182S+E189P+N194F+L205Y+Y242F+E259G+K280Y+G303R+E390A+G475K, H1*+G7A+D118T+W140Y+R180*+S181*+T182S+E189P+N194F+L205Y+Y242F+E259G+N279S+G303R+E390A+G475K, H1*+G7A+D118T+W140Y+R180*+S181*+T182S+E189P+N194F+L205Y+Y242F+E259G+N279S+K280Y+E390A+G475K, H1*+G7A+D118T+W140Y+R180*+S181*+T182S+E189P+N194F+L205Y+Y242F+E259G+N279S+K280Y+G303R+G475K, H1*+G7A+D118T+W140Y+R180*+S181*+T182S+E189P+N194F+L205Y+Y242F+E259G+N279S+K280Y+G303R+E390A, H1*+G7A+D118T+W140Y+R180*+S181*+T182S+E189P+N194F+L205V+Y242F+E259G+N279S+K280Y+G303R+E390A+G475K, H1*+G7A+D118T+W140Y+R180*+S181*+T182S+E189P+N194F+L205N+Y242F+E259G+N279S+K280Y+G303R+E390A+G475K, H1*+G7A+D118T+W140Y+R180*+S181*+T182S+E189P+N194F+L205Q+Y242F+E259G+N279S+K280Y+G303R+E390A+G475K, H1*+G7A+D118T+W140Y+R180*+S181*+T182S+A185Q+E189P+N194F+L205Y+Y242F+E259G+N279S+K280Y+G303R+E390A+G475K, H1*+G7A+D118T+W140Y+R180*+S181*+T182S+E189P+N194F+L205Y+Y242F+E259G+F266Y+N279S+K280Y+G303R+E390A+G475K, H1*+G7A+D118T+W140Y+R180*+S181*+A185Q+E189P+N194F+L205Y+Y242F+E259G+N279S+K280Y+G303R+E390A+G475K, H1*+G7A+D118T+W140Y+R180*+S181*+T182S+A185Q+E189P+N194F+L205Y+Y242F+E259G+F266Y+N279S+K280Y+G303R+E390A+G475K, H1*+G7A+D118T+W140Y+R180*+S181*+T182S+A185Q+E189P+N194F+L205N+Y242F+E259G+F266Y+N279S+K280Y+G303R+E390A+G475K, H1*+G7A+D118T+W140Y+R180*+S181*+T182S+A185D+E189P+N194F+Y242F+E259G+F266Y+N279S+K280Y+G303R+E390A+G475K, H1*+G7A+D118T+W140Y+R180*+S181*+T182S+A185D+E189P+Y242F+S243Q+E259G+F266Y+N279S+K280Y+G303R+E390A+G475K, H1*+G7A+D118T+W140Y+R180Q+S181*+T182*+A185Q+E189P+Y242F+S243Q+E259G+F266Y+N279S+K280Y+G303R+E390A+G475K, H1*+G7A+D118T+W140Y+R180*+S181*+T182S+A185Q+E189P+Y242F+E259G+F266Y+N279S+K280Y+G303R+E390A+G475K, H1*+G7A+D118T+W140Y+R180*+S181*+T182S+A185Q+E189P+L205Y+Y242F+E259G+F266Y+N279S+K280Y+G303R+E390A+G475K, H1*+G7A+D118T+W140Y+R180*+S181*+T182S+A185D+E189P+L205Y+Y242F+E259G+F266Y+N279S+K280Y+G303R+E390A+G475K, H1*+G7A+D118T+W140Y+R180Q+S181*+T182*+A185Q+E189P+N194F+L205Y+Y242F+E259G+F266Y+N279S+K280Y+G303R+E390A+G475K, H1*+N3D+G7A+D118T+W140Y+R180*+S181*+T182S+E189P+N194F+L205Y+Y242F+E259G+N279S+K280Y+S302T+E390A+H420A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+T40G+D118T+W140Y+R180*+S181*+T182S+E189P+N194F+L205Y+Y242F+E259G+N279S+K280Y+G303R+E390A+G476A, H1*+G7A+T40G+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+G303R+N394D+N422V+G475K, H1*+G7A+Q98L+D118T+W140Y+R180*+S181*+T182S+E189P+N194F+L205Y+Y242F+E259G+N279S+K280Y+G303R+N394D+H420A+G475K, H1*+G7A+D118T+W140Y+R180*+S181*+T182S+E189P+N194F+L205Y+Y242F+E259G+M260D+N279S+K280Y+G303R+Y362V+E390A+G475K, H1*+G7A+D118T+Y135E+W140Y+R180*+S181*+T182S+E189P+N194F+L205Y+Y242F+E259G+F261P+N279S+K280Y+G303R+Y362V+E390A+G475K, H1*+G7A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+Y135E+W140Y+R180*+S181*+T182S+E189P+N194F+L205Y+T224F+Y242F+E259G+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+E259G+F261P+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+Y371N+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+N474F, H1*+G7A+D118T+Y135E+W140Y+R180*+S181*+T182S+E189P+N194F+L205Y+Y242F+E259G+N279S+K280Y+G303R+P321Y+E390A+G475K, H1*+G7A+D118T+S132D+W140Y+R180*+S181*+T182S+E189P+N194F+L205Y+Y242F+E259G+N279S+K280Y+G303R+P321Y+E390A+G475K, H1*+N3G+G7A+D118T+Q138V+W140Y+R180*+S181*+T182S+E189P+N194F+L205Y+Y242F+E259G+N279S+K280Y+G303R+E390A+H420A+G476A, H1*+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+T256D+N279S+K280Y+G303R+E390A+N470M+G475K, H1*+N3D+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+S132D+W140Y+R180*+S181*+T182S+E189P+N194F+L205Y+Y242F+E259G+N279S+K280Y+G303R+Y362V+E390A+N474L, H1*+G7A+D118T+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+S302T+Y362V+E390A+G475K, H1*+G7A+D118T+W140Y+R180*+S181*+T182S+E189P+N194F+L205Y+Y242F+E259G+N279S+K280Y+G303R+Y362V+E390A+G476A, H1*+G7A+D118T+W140Y+R180*+S181*+T182S+E189P+N194F+L205Y+Y242F+E259G+M260D+N279S+K280Y+G303R+E390A+N474L, H1*+G7A+D118T+W140Y+R180*+S181*+T182S+E189P+N194F+L205Y+T224Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+Y362V+E390A+G475K, H1*+G7A+D118T+W140Y+R180*+S181*+T182S+E189P+N194F+L205Y+Y242F+E259G+M260W+N279S+K280Y+S302T+E390A+G475K, H1*+G7A+D118T+W140Y+R180*+S181*+T182S+E189P+N194F+L205Y+T224Y+Y242F+E259G+N279S+K280Y+G303R+E390A+G476A, H1*+G7A+D118T+W140Y+R180*+S181*+T182S+E189P+N194F+L205Y+Y242F+E259G+M260D+N279S+K280Y+G303R+E390A+N474F, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+M260D+N279S+K280Y+G303R+E390A+H420A+G475K, H1*+G7A+D118T+Y135E+W140Y+R180*+S181*+T182S+E189P+N194F+L205Y+Y242F+E259G+N279S+K280Y+G303R+N394W+H420A+G475K, H1*+N3D+G7A+D118T+W140Y+R180*+S181*+T182S+E189P+N194F+L205Y+Y242F+E259G+N279S+K280Y+G303R+N394D+H420A+G475K, H1*+G7A+D118T+Q138V+W140Y+R180*+S181*+T182S+E189P+N194F+L205Y+Y242F+E259G+N279S+K280Y+G303R+E390A+H420Q+G475K, H1*+G7A+R37N+D118T+W140Y+R180*+S181*+T182S+E189P+N194F+L205Y+Y242F+E259G+N279S+K280Y+G303R+E390A+H420P+G476H, H1*+G7A+R37A+Q98L+D118T+W140Y+R180*+S181*+T182S+E189P+N194F+L205Y+Y242F+E259G+N279S+K280Y+S302Y+E390A+G475K, H1*+G7A+R37A+D118T+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+N279S+K280Y+G303R+E390A+H420P+G475K, H1*+G7A+D118T+W140Y+R180*+S181*+T182S+E189P+N194F+L205Y+Y242F+E259G+N279S+K280Y+G303R+E390A+N470M+G476A, H1*+G7A+D118T+W140Y+R180*+S181*+T182S+E189P+N194F+L205Y+Y242F+E259G+M260D+N279S+K280Y+G303R+E390A+H420P+G476A, H1*+G7A+D118T+W140Y+R180*+S181*+T182S+E189P+N194F+L205Y+Y242F+E259G+N279S+K280Y+S302T+E390A+H420A+G475K, H1*+G7A+R37A+D118T+W140Y+R180*+S181*+T182S+E189P+N194F+L205Y+Y242F+E259G+N279S+K280Y+S302Y+E390A+H420A+G475K, H1*+G7A+D118T+Y135E+W140Y+R180*+S181*+T182S+E189P+N194F+L205Y+Y242F+E259G+N279S+K280Y+S302T+Y371N+E390A+G475K, H1*+G7A+R37N+D118T+S132D+W140Y+R180*+S181*+T182S+E189P+N194F+L205Y+Y242F+E259G+N279S+K280Y+G303R+E390A+N470M+G475K, H1*+G7A+D118T+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+N279S+K280Y+G303R+H323W+E390A+H420A+N474L, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+N394W+H420P+G475K, H1*+G7A+D118T+W140Y+R180*+S181*+T182S+E189P+N194F+L205Y+Y242F+E259G+N279S+K280Y+S302T+H323W+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+Y135E+W140Y+R180*+S181*+T182S+E189P+N194F+L205Y+Y242F+E259G+N279S+K280Y+G303R+E390A+R414M+G476H, H1*+G7A+R37N+D118T+W140Y+R180*+S181*+T182S+E189P+N194F+L205Y+Y242F+E259G+N279S+K280Y+S302Y+E390A+H420A+G475K, H1*+G7A+R37A+D118T+W140Y+R180*+S181*+T182S+E189P+N194F+L205Y+Y242F+E259G+N279S+K280Y+S302T+E390A+H420A+G476A, H1*+G7A+R37N+D118T+W140Y+R180*+S181*+T182S+E189P+N194F+L205Y+Y242F+E259G+N279S+K280Y+S302Y+E390A+H420A+G475K, H1*+G7A+R37A+D118T+W140Y+R180*+S181*+T182S+E189P+N194F+L205Y+Y242F+E259G+N279S+K280Y+S302Y+E390A+N474L, H1*+G7A+R37A+D118T+W140Y+R180*+S181*+T182S+E189P+N194F+L205Y+Y242F+E259G+N279S+K280Y+S302Y+E390A+G475K, H1*+N3D+G7A+R37A+D118T+W140Y+R180*+S181*+T182S+E189P+N194F+L205Y+Y242F+E259G+N279S+K280Y+S302Y+N394D+H420A+G475K, H1*+G7A+R37A+D118T+W140Y+R180*+S181*+T182S+E189P+N194F+L205Y+Y242F+E259G+N279S+K280Y+S302Y+E390A+H420A+G476H, H1*+G7A+R37A+D118T+W140Y+R180*+S181*+T182S+E189P+N194F+L205Y+Y242F+E259G+N279S+K280Y+S302Y+N394D+H420A+G475K, H1*+G7A+R37A+D118T+W140Y+R180*+S181*+T182S+E189P+N194F+L205Y+Y242F+E259G+N279S+K280Y+S302Y+E390A+N474L, H1*+G7A+R37N+D118T+W140Y+R180*+S181*+T182S+E189P+N194F+L205Y+Y242F+E259G+N279S+K280Y+S302T+E390A+H420A+G475K, H1*+G7A+R37A+D118T+W140Y+R180*+S181*+T182S+E189P+N194F+L205Y+Y242F+E259G+N279S+K280Y+S302Y+E390A+H420A+G475K, H1*+G7A+R37N+D118T+W140Y+R180*+S181*+T182S+E189P+N194F+L205Y+Y242F+E259G+N279S+K280Y+S302T+E390A+H420A+G475K, H1*+G7A+R37A+D118T+W140Y+R180*+S181*+T182S+E189P+N194F+L205Y+Y242F+E259G+N279S+K280Y+S302T+E390A+H420A+G475K, H1*+G7A+D118T+W140Y+R180*+S181*+T182S+E189P+N194F+L205Y+Y242F+E259G+N279S+K280Y+S302T+N394W+H420A+G475K, H1*+G7A+R37A+D118T+W140Y+R180*+S181*+T182S+E189P+N194F+L205Y+Y242F+E259G+N279S+K280Y+S302Y+N394D+G475K, H1*+G7A+R37N+D118T+W140Y+R180*+S181*+T182S+E189P+N194F+L205Y+Y242F+E259G+N279S+K280Y+S302Y+E390A+H420A+G476H, H1*+G7A+R37N+D118T+W140Y+R180*+S181*+T182S+E189P+N194F+L205Y+Y242F+E259G+N279S+K280Y+S302T+N394D+G475K, H1*+G7A+R37A+D118T+W140Y+R180*+S181*+T182S+E189P+N194F+L205Y+Y242F+E259G+N279S+K280Y+S302T+E390A+G475K, H1*+G7A+R37N+D118T+W140Y+R180*+S181*+T182S+E189P+N194F+L205Y+Y242F+E259G+N279S+K280Y+S302T+N394D+H420A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+D118T+W140Y+R180*+S181*+T182S+E189P+N194F+L205Y+Y242F+E259G+N279S+K280Y+S302T+E390A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302Y+E390A+N474L, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302Y+E390A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3D+G7A+R37A+D118T+W140Y+R180*+S181*+T182S+E189P+N194F+L205Y+Y242F+E259G+N279S+K280Y+G303R+E390A+G475K, H1*+N3G+G7A+R37N+D118T+W140Y+R180*+S181*+T182S+E189P+N194F+L205Y+Y242F+E259G+N279S+K280Y+G303R+N394W+H420A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3D+G7A+R37N+D118T+W140Y+R180*+S181*+T182S+E189P+N194F+L205Y+Y242F+E259G+N279S+K280Y+G303R+G361M+H420A+G475K, H1*+N3D+G7A+R37A+D118T+W140Y+R180*+S181*+T182S+E189P+N194F+L205Y+Y242F+E259G+N279S+K280Y+G303R+E390A+H420A+G475K, H1*+G7A+R37A+D118T+W140Y+R180*+S181*+T182S+E189P+N194F+L205Y+Y242F+E259G+M260D+N279S+K280Y+G303R+N394W+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+R37A+D118T+W140Y+R180*+S181*+T182S+E189P+N194F+L205Y+T224F+Y242F+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+Y242F+E259G+M260D+N279S+K280Y+G303R+N394D+G475K, H1*+G7A+R37N+D118T+W140Y+R180*+S181*+T182S+E189P+N194F+L205Y+Y242F+E259G+M260D+N279S+K280Y+G303R+N394D+G475K, H1*+G7A+D118T+W140Y+R180*+S181*+T182S+E189P+N194F+L205Y+Y242F+E259G+M260D+N279S+K280Y+G303R+H420A+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+R37N+D118T+W140Y+R180*+S181*+T182S+E189P+N194F+L205Y+T224Y+Y242F+N279S+K280Y+G303R+E390A+G475K, H1*+G7A+D118T+W140Y+R180*+S181*+T182S+E189P+N194F+L205Y+Y242F+E259G+N279S+K280Y+G303R+N394W+H420A+G475K, H1*+G7A+D118T+W140Y+R180*+S181*+T182S+E189P+N194F+L205Y+Y242F+E259G+N279S+K280Y+G303R+N394W+H420A+N474L, H1*+G7A+D118T+W140Y+P146H+R180*+S181*+T182S+E189P+N194F+L205Y+Y242F+E259G+N279S+K280Y+S302T+E390A+G476A, H1*+G7A+D118T+W140Y+P146H+R180*+S181*+T182S+E189P+N194F+L205Y+Y242F+E259G+N279S+K280Y+S302Y+E390A+N474L, H1*+G7A+D118T+W140Y+R180*+S181*+T182S+A185Q+E189P+N194F+Y242F+E259G+F266Y+N279S+K280Y+G303R+E390A+G475K, H1*+G7A+D118T+W140Y+R180*+S181*+T182S+A185Q+E189P+N194F+L205Q+Y242F+E259G+F266Y+N279S+K280Y+G303R+E390A+G475K, H1*+G7A+D118T+W140Y+R180Q+S181*+T182*+A185Q+E189P+N194F+Y242F+E259G+F266Y+N279S+K280Y+G303R+E390A+G475K, H1*+G7A+D118T+W140Y+R180*+S181*+T182S+A185Q+E189P+Y242F+S243Q+E259G+F266Y+N279S+K280Y+G303R+E390A+G475K, H1*+N3G+G7A+V104M+D118T+W140Y+R180*+S181*+T182S+E189P+N194F+L205Y+Y242F+E259G+N279S+K280Y+S302Y+N394W+G475K, H1*+N3D+G7A+D118T+W140Y+R180*+S181*+T182S+E189P+N194F+L205Y+Y242F+E259G+N279S+K280Y+G303R+E390A+G475K, H1*+N3G+G7A+D118T+W140Y+R180*+S181*+T182S+E189P+N194F+L205Y+Y242F+E259G+N279S+K280Y+G303R+N394W+H420A+G475K, H1*+N3D+G7A+D118T+W140Y+R180*+S181*+T182S+E189P+N194F+L205Y+Y242F+E259G+N279S+K280Y+G303R+E390A+H420A+G475K, H1*+N3D+G7A+D118T+S132D+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+N279S+K280Y+G303R+E390A+N470M+G475K, H1*+G7A+D118T+W140Y+R180*+S181*+T182S+E189P+N194F+L205Y+Y242F+E259G+N279S+K280Y+S302Y+Y371N+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+G475K, H1*+G7A+D118T+Y135E+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+T224F+Y242F+E259G+N279S+K280Y+S302T+E390A+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+N394W+G475K, H1*+G7A+D118T+W140Y+R180*+S181*+T182S+E189P+N194F+L205Y+Y242F+E259G+M260D+N279S+K280Y+G303R+E390A+G475K, H1*+G7A+R37A+D118T+W140Y+R180*+S181*+T182S+E189P+N194F+L205Y+Y242F+E259G+M260W+N279S+K280Y+G303R+E390A+N470M+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+N394D+G475K, H1*+G7A+R37N+D118T+W140Y+R180*+S181*+T182S+E189P+N194F+L205Y+Y242F+E259G+M260W+N279S+K280Y+G303R+E390A+H420A+G475K, H1*+G7A+D118T+W140Y+R180*+S181*+T182S+E189P+N194F+L205Y+Y242F+E259G+M260D+N279S+K280Y+G303R+E390A+N470M+G475K, H1*+G7A+R37A+D118T+W140Y+R180*+S181*+T182S+E189P+N194F+L205Y+Y242F+E259G+M260D+N279S+K280Y+G303R+N394W+G475K, H1*+G7A+R37A+D118T+W140Y+R180*+S181*+T182S+E189P+N194F+L205Y+Y242F+E259G+N279S+K280Y+G303R+E390A+H420A+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+R37A+D118T+W140Y+R180*+S181*+T182S+E189P+N194F+L205Y+T224Y+Y242F+E259G+N279S+K280Y+G303R+N394W+G475K, H1*+G7A+R37N+D118T+W140Y+R180*+S181*+T182S+E189P+N194F+L205Y+Y242F+E259G+M260D+N279S+K280Y+G303R+N394W+H420A+G475K, H1*+G7A+R37A+D118T+W140Y+R180*+S181*+T182S+E189P+N194F+L205Y+Y242F+E259G+M260D+N279S+K280Y+G303R+E390A+N470M+G475K, H1*+G7A+R37A+D118T+W140Y+R180*+S181*+T182S+E189P+N194F+L205Y+Y242F+T256D+N279S+K280Y+G303R+E390A+G475K, H1*+G7A+R37A+D118T+W140Y+R180*+S181*+T182S+E189P+N194F+L205Y+Y242F+E259G+F261P+N279S+K280Y+G303R+E390A+G475K, H1*+G7A+R37A+D118T+W140Y+R180*+S181*+T182S+E189P+N194F+L205Y+Y242F+E259G+M260D+N279S+K280Y+G303R+N394D+H420A+G475K, H1*+G7A+R37A+D118T+W140Y+R180*+S181*+T182S+E189P+N194F+L205Y+Y242F+E259G+M260D+N279S+K280Y+G303R+N394D+G475K, H1*+G7A+R37N+D118T+W140Y+N174K+R180*+S181*+T182S+E189P+N194F+L205Y+Y242F+E259G+M260D+N279S+K280Y+G303R+N394W+G475K, H1*+G7A+R37A+D118T+W140Y+R180*+S181*+T182S+E189P+N194F+L205Y+T224Y+Y242F+E259G+N279S+K280Y+G303R+E390A+G475K, H1*+G7A+R37A+D118T+W140Y+R180*+S181*+T182S+E189P+N194F+L205Y+T224Y+Y242F+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+H420A+G475K, H1*+N3D+G7A+D118T+W140Y+R180*+S181*+T182S+E189P+N194F+L205Y+Y242F+E259G+M260D+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3G+G7A+R37N+D118T+W140Y+R180*+S181*+T182S+E189P+N194F+L205Y+Y242F+E259G+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+E259G+N279+K280Y+S302T+E390A+N470M+G475K, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+S132L+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+H420A+G476A, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+N394D+G475K, H1*+G7A+D118T+W140Y+R180*+S181*+T182S+E189P+N194F+L205Y+T224F+Y242F+E259G+N279S+K280Y+G303R+E390A+H420A+G475K, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+G475K, H1*+G7A+D118T+S132D+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+N394W+G475K, H1*+G7A+D118T+S132L+W140Y+R180*+S181*+T182S+E189P+N194F+L205Y+T224F+Y242F+E259G+N279S+K280Y+S302Y+E390A+G475K, H1*+G7A+D118T+Q138V+W140Y+R180*+S181*+T182S+E189P+N194F+L205Y+Y242F+E259G+N279S+K280Y+S302Y+N394D+G475K, H1*+G7A+D118T+S132L+W140Y+R180*+S181*+T182S+E189P+N194F+L205Y+Y242F+E259G+N279S+K280Y+S302Y+N394W+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+H420A+G476H, H1*+G7A+D118T+W140Y+R180*+S181*+T182S+E189P+N194F+L205Y+T224F+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+N394D+G475K, H1*+G7A+D118T+Q138V+W140Y+R180*+S181*+T182S+E189P+N194F+L205Y+Y242F+E259G+N279S+K280Y+S302Y+E390A+G475K, H1*+G7A+D118T+Q138V+W140Y+R180*+S181*+T182S+E189P+N194F+L205Y+Y242F+E259G+N279S+K280Y+S302Y+E390A+G475K, H1*+G7A+D118T+S132L+W140Y+R180*+S181*+T182S+E189P+N194F+L205Y+Y242F+E259G+N279S+K280Y+S302Y+N394W+G475K, H1*+G7A+D118T+Q138V+W140Y+R180*+S181*+T182S+E189P+N194F+L205Y+T224F+Y242F+E259G+N279S+K280Y+S302Y+E390A+G475K, H1*+G7A+Q98L+D118T+W140Y+R180*+S181*+T182S+E189P+N194F+L205Y+Y242F+E259G+N279S+K280Y+G303R+E390A+G475K, H1*+G7A+R37N+D118T+W140Y+R180*+S181*+T182S+E189P+N194F+L205Y+Y242F+T256D+E259G+N279S+K280Y+G303R+E390A+G475K, H1*+G7A+R37A+D118T+W140Y+R180*+S181*+T182S+E189P+N194F+L205Y+Y242F+E259G+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+N394D+G475K, N3G+G7A+R37V+D118T+W140M+R180*+S181*+T182S+E189P+N194F+L205Y+Y242F+E259G+N279S+K280Y+G303R+E390A+H420A+G475K, H1*+G7A+R37N+D118T+W140Y+R180*+S181*+T182S+E189P+N194F+L205Y+Y242F+E259G+N279S+K280Y+G303R+H323W+N394D+G475K, H1*+G7A+R37A+D118T+W140Y+R180*+S181*+T182S+E189P+N194F+L205Y+Y242F+E259G+N279S+K280Y+G303R+P321Y+L354V+N394D+G475K, H1*+G7A+R37N+D118T+W140Y+R180*+S181*+T182S+E189P+N194F+L205Y+Y242F+E259G+N279S+K280Y+G303R+H323W+Q360M+E390A+G475K, H1*+N3D+G7A+D118T+W140Y+R180*+S181*+T182S+E189P+N194F+L205Y+Y242F+E259G+N279S+K280Y+G303R+H323W+E390A+G475K, H1*+N3D+G7A+D118T+W140Y+R180*+S181*+T182S+E189P+N194F+L205Y+Y242F+E259G+N279S+K280Y+G303R+H323W+E390A+G475K, H1*+G7A+D118T+W140Y+R180*+S181*+T182S+A185D+E189P+Y242F+E259G+F266Y+N279S+K280Y+G303R+E390A+G475K, H1*+G7A+D118T+W140Y+R180Q+S181*+T182*+A185Q+E189P+Y242F+E259G+F266Y+N279S+K280Y+G303R+E390A+G475K, H1*+G7A+D118T+W140Y+R180*+S181*+T182S+E189P+N194F+Y242F+E259G+N279S+K280Y+S302T+E390A+G475K, H1*+G7A+D118T+W140Y+R180*+S181*+T182S+E189P+N194F+Y242F+E259G+M260D+N279S+K280Y+S302T+E390A+G475K, H1*+G7A+R37N+D118T+W140Y+R180*+S181*+T182S+E189P+N194F+Y242F+E259G+N279S+K280Y+S302Y+E390A+G475K, H1*+G7A+D118T+W140Y+R180*+S181*+T182S+E189P+N194F+Y242F+E259G+M260W+N279S+K280Y+G303R+E390A+G476A, H1*+G7A+W140Y+R180*+S181*+E189P+N194F+Y242F+E259G+F266Y+N279S+K280Y+S302T+E390A+G475K, H1*+G7A+D118T+W140Y+R180*+S181*+T182D+E189P+N194F+Y242F+E259G+N279S+K280Y+G303R+E390A+G476A, H1*+G7A+D118T+S132L+W140Y+R180*+S181*+T182D+E189P+N194F+L205Y+Y242F+E259G+M260D+N279S+K280Y+G303R+E390A+G475K, H1*+G7A+D118T+W140Y+R180*+S181*+T182D+E189P+N194F+Y242F+E259G+M260D+N279S+K280Y+S302T+E390A+G475K, H1*+N3D+G7A+D118T+W140Y+R180*+S181*+T182D+E189P+N194F+Y242F+E259G+N279S+K280Y+S302T+E390A+H420A+G475K, H1*+G7A+R37N+D118T+W140Y+R180*+S181*+T182D+E189P+N194F+Y242F+E259G+N279S+K280Y+S302Y+E390A+G475K, H1*+G7A+D118T+W140Y+R180*+S181*+T182S+A185Q+E189P+N194F+L205Y+T224Y+Y242F+N279S+K280Y+G303R+E390A+G476A, H1*+G7A+D118T+W140Y+R180*+S181*+T182S+A185Q+E189P+N194F+Y242F+E259G+M260D+N279S+K280Y+S302Y+E390A+N470M+G475K, H1*+G7A+D118T+W140Y+R180*+S181*+T182S+A185Q+E189P+N194F+Y242F+E259G+N279S+K280Y+G303R+E390A+G476A, H1*+G7A+D118T+W140Y+R180*+S181*+T182S+A185Q+E189P+N194F+Y242F+E259G+M260D+N279S+K280Y+S302T+E390A+G475K, H1*+N3D+G7A+D118T+W140Y+R180*+S181*+T182S+A185Q+E189P+N194F+Y242F+E259G+N279S+K280Y+S302T+E390A+H420A+G475K, H1*+G7A+R37N+D118T+W140Y+R180*+S181*+T182S+A185Q+E189P+N194F+Y242F+E259G+N279S+K280Y+S302Y+E390A+G475K, H1*+G7A+D118T+W140Y+R180*+S181*+T182S+A185Q+E189P+N194F+L205Y+Y242F+E259G+M260W+N279S+K280Y+G303R+E390A+G476A, H1*+G7A+D118T+W140Y+R180*+S181*+T182S+E189P+N194F+T224Y+Y242F+F266Y+N279S+K280Y+G303R+E390A+G476A, H1*+G7A+D118T+W140Y+R180*+S181*+T182S+E189P+N194F+Y242F+E259G+M260D+F266Y+N279S+K280Y+S302Y+E390A+N470M+G475K, H1*+G7A+D118T+W140Y+R180*+S181*+T182S+E189P+N194F+Y242F+E259G+F266Y+N279S+K280Y+S302T+E390A+G475K, H1*+G7A+D118T+W140Y+R180*+S181*+T182S+E189P+N194F+Y242F+E259G+F266Y+N279S+K280Y+G303R+E390A+G476A, H1*+G7A+D118T+S132L+W140Y+R180*+S181*+T182S+E189P+N194F+Y242F+E259G+M260D+F266Y+N279S+K280Y+G303R+E390A+G475K, H1*+G7A+D118T+W140Y+R180*+S181*+T182S+E189P+N194F+Y242F+E259G+M260D+F266Y+N279S+K280Y+S302T+E390A+G475K, H1*+G7A+D118T+W140Y+R180*+S181*+T182S+E189P+N194F+Y242F+E259G+M260W+F266Y+N279S+K280Y+G303R+E390A+G476A, H1*+G7A+D118T+W140Y+R180*+S181*+T182D+E189P+N194F+Y242F+E259G+F266Y+N279S+K280Y+S302T+E390A+G475K, H1*+G7A+D118T+W140Y+R180*+S181*+T182D+E189P+N194F+Y242F+E259G+M260D+F266Y+N279S+K280Y+S302T+E390A+G475K, H1*+N3D+G7A+D118T+W140Y+R180*+S181*+T182D+E189P+N194F+Y242F+E259G+F266Y+N279S+K280Y+S302T+E390A+H420A+G475K, H1*+G7A+R37N+D118T+W140Y+R180*+S181*+T182D+E189P+N194F+Y242F+E259G+F266Y+N279S+K280Y+S302Y+E390A+G475K, H1*+G7A+D118T+W140Y+R180*+S181*+T182S+A185Q+E189P+N194F+T224Y+Y242F+F266Y+N279S+K280Y+G303R+E390A+G476A, H1*+G7A+D118T+W140Y+R180*+S181*+T182S+A185Q+E189P+N194F+Y242F+E259G+F266Y+N279S+K280Y+S302T+E390A+G475K, H1*+G7A+D118T+W140Y+R180*+S181*+T182S+A185Q+E189P+N194F+Y242F+E259G+F266Y+N279S+K280Y+G303R+E390A+G476A, H1*+G7A+D118T+S132L+W140Y+R180*+S181*+T182S+A185Q+E189P+N194F+L205Y+Y242F+E259G+M260D+F266Y+N279S+K280Y+G303R+E390A+G475K, H1*+N3D+G7A+D118T+W140Y+R180*+S181*+T182S+A185Q+E189P+N194F+Y242F+E259G+F266Y+N279S+K280Y+S302T+E390A+H420A+G475K, H1*+G7A+D118T+W140Y+R180*+S181*+T182S+A185Q+E189P+N194F+Y242F+E259G+M260W+F266Y+N279S+K280Y+G303R+E390A+G476A, H1*+G7A+W140Y+R180*+S181*+E189P+N194F+Y242F+E259G+M260D+N279S+K280Y+S302T+E390A+G475K, H1*+N3D+G7A+W140Y+R180*+S181*+E189P+N194F+Y242F+E259G+F266Y+N279S+K280Y+S302T+E390A+H420A+G475K, H1*+G7A+R37N+W140Y+R180*+S181*+E189P+N194F+Y242F+E259G+F266Y+N279S+K280Y+S302Y+E390A+G475K, H1*+G7A+W140Y+R180*+S181*+E189P+N194F+Y242F+E259G+F266Y+N279S+K280Y+G303R+E390A+G476A, H1*+G7A+D118T+W140Y+R180*+S181*+T182S+E189P+N194F+Y242F+E259G+M260D+N279S+K280Y+S302Y+E390A+N470M+G475K, H1*+G7A+D118T+W140Y+R180*+S181*+T182D+E189P+N194F+Y242F+E259G+M260D+N279S+K280Y+S302Y+E390A+N470M+G475K, H1*+G7A+D118T+W140Y+R180*+S181*+T182D+E189P+N194F+Y242F+E259G+M260W+N279S+K280Y+G303R+E390A+G476A, H1*+G7A+D118T+W140Y+R180*+S181*+T182S+A185Q+E189P+N194F+Y242F+E259G+M260W+N279S+K280Y+G303R+E390A+G476A, H1*+G7A+D118T+W140Y+R180*+S181*+T182S+E189P+N194F+T224Y+Y242F+N279S+K280Y+G303R+E390A+G476A, H1*+G7A+D118T+W140Y+R180*+S181*+T182D+E189P+N194F+T224Y+Y242F+N279S+K280Y+G303R+E390A+G476A, H1*+G7A+D118T+W140Y+R180*+S181*+T182S+A185Q+E189P+N194F+T224Y+Y242F+N279S+K280Y+G303R+E390A+G476A, H1*+G7A+D118T+W140Y+R180*+S181*+T182S+A185Q+E189P+N194F+Y242F+E259G+N279S+K280Y+S302T+E390A+G475K, H1*+G7A+D118T+W140Y+R180*+S181*+T182D+E189P+N194F+Y242F+E259G+F266Y+N279S+K280Y+G303R+E390A+G476A, H1*+G7A+D118T+W140Y+R180*+S181*+T182D+E189P+N194F+Y242F+E259G+M260D+N279S+K280Y+S302Y+E390A+N470M+G475K, H1*+G7A+D118T+W140Y+R180*+S181*+T182D+A185Q+E189P+N194F+Y242F+E259G+F266Y+N279S+K280Y+G303R+E390A+G475K, H1*+G7A+D118T+W140Y+R180*+S181*+T182D+A185Q+Y242F+S243Q+E259G+F266Y+N279S+G303R+E390A+G475K, H1*+G7A+D118T+W140Y+R180*+S181*+T182D+N194F+Y242F+S243Q+E259G+F266Y+N279S+G303R+E390A+G475K, H1*+G7A+D118T+W140Y+R180*+S181*+T182S+A185Q+E189P+N196I+Y242F+E259G+F266Y+N279S+K280Y+G303R+E390A+G475K, H1*+G7A+D118T+W140Y+R180*+S181*+T182S+A185D+E189P+Y242F+E259G+F266Y+N279S+K280Y+G303R+E390A+H420Y+G475K, H1*+G7A+D118T+W140Y+R180*+S181*+A185Q+E189P+N194F+L205Y+Y242F+E259G+F266Y+N279S+K280Y+G303R+E390A+G475K, H1*+G7A+W140Y+R180*+S181*+E189P+N194F+Y242F+E259G+M260W+N279S+K280Y+G303R+E390A+G476A, H1*+G7A+D118T+W140Y+R180*+S181*+T182S+E189P+N194F+Y242F+E259G+M260D+N279S+K280Y+S302Y+E390A+N470M+G475K, H1*+G7A+D118T+W140Y+R180*+S181*+T182D+E189P+N194F+T224Y+Y242F+N279S+K280Y+G303R+E390A+G476A, H1*+G7A+D118T+W140Y+R180*+S181*+T182S+E189P+N194F+T224Y+Y242F+N279S+K280Y+G303R+E390A+G476A, H1*+G7A+D118T+W140Y+R180*+S181*+T182D+A185Q+E189P+N194F+T224Y+Y242F+N279S+K280Y+G303R+E390A+G476A, H1*+G7A+D118T+W140Y+R180*+S181*+T182D+A185Q+N194F+Y242F+E259G+N279S+K280Y+S302T+E390A+G475K, H1*+G7A+R37N+D118T+W140Y+R180*+S181*+T182S+A185Q+E189P+N194F+L205Y+Y242F+E259G+N279S+K280Y+S302Y+E390A+G475K, H1*+G7A+D118T+W140Y+R180*+S181*+T182S+A185Q+E189P+N194F+L205Y+Y242F+E259G+M260D+N279S+K280Y+S302T+E390A+G475K, H1*+N3D+G7A+D118T+W140Y+R180*+S181*+T182D+E189P+N194F+L205Y+Y242F+E259G+N279S+K280Y+S302T+E390A+H420A+G475K, H1*+G7A+W140Y+R180*+S181*+E189P+N194F+Y242F+E259G+M260D+N279S+K280Y+S302Y+E390A+N470A+G475K, H1*+G7A+S132L+W140Y+R180*+S181*+E189P+N194F+Y242F+E259G+M260D+N279S+K280Y+G303R+E390A+G475K, H1*+G7A+A31S+R37N+D118T+W140Y+R180*+S181*+T182S+A185Q+E189P+N194F+Y242F+E259G+F266Y+N279S+K280Y+S302Y+E390A+G475K, H1*+G7A+D118T+W140Y+R180*+S181*+T182S+A185Q+E189P+N194F+Y242F+E259G+M260W+N279S+K280Y+G303R+E390A+G476A, H1*+G7A+D118T+W140Y+R180*+S181*+T182D+E189P+N194F+L205Y+T224Y+Y242F+F266Y+N279S+K280Y+G303R+E390A+G476A, H1*+G7A+D118T+W140Y+R180*+S181*+T182S+A185Q+E189P+N194F+Y242F+E259G+N279S+K280Y+S302T+E390A+G475K, R180*+S181*, T40N+F116L+D123N+N126D+N128Y+T131I+F155W+R180*+S181*+T182D+E189P+F202Y+K457N+P458S+G475K+G476E, A60V+Q98L+G110K+R180*+S181*+A383D, Q98L+G110K+S132D+Y135E+R180*+S181*+T315A, G7H+Q98L+G110K+R180*+S181*, G7H+W167P+R180*+S181*+G455R, Q98L+V103A+G110K+R180*+S181*, T114K+R180*+S181*, E169V+R180*+S181*, R180*+S181*+W188C, W167D+R180*+S181*, W167S+R180*+S181*, W167E+R180*+S181*, W167P+R180*+S181*, R180*+S181*+E193I+H323N, R180*+S181*+A203F, W159L+R180*+S181*, W159H+R180*+S181*, K178A+R180*+S181*, A111L+R180*+S181*, G110K+R180*+S181*, and A111G+R180*+S181*, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO:1, 2, 3, 4, 5, 6, 7, 8, 9, 10, 11, 12, 13 or 14 and wherein the variant has alpha-amylase activity.
Paragraph 10. The alpha-amylase variant according to any one of preceding paragraphs, wherein said variant has improved property, wherein said improved property is improved wash performance for a measure of increased specific activity of Improvement Factor (IF) of >1.0.
Paragraph 11. The alpha-amylase variant according to any one of preceding paragraphs, wherein said variant has improved property, wherein said improved property is improved wash performance for a measure of increased specific activity of Improvement Factor (IF) of >1.0 compared to said parent polypeptide.
Paragraph 12. The alpha-amylase variant according to any one of preceding paragraphs, wherein said variant has improved property, wherein said improved property is improved wash performance for a measure of increased specific activity of Improvement Factor (IF) of >1.0 compared to said parent polypeptide of SEQ ID NO: 2 and/or SEQ ID NO: 3.
Paragraph 13. The alpha-amylase variant according to any one of preceding paragraphs, wherein the number of modification is 1 to 20, e.g., 1 to 10 and 1 to 5, such as 1, 2, 3, 4, 5, 6, 7, 8, 9, 10, 11, 12, 13, 14, 15, 16, 17, 18, 19, or 20 modification.
Paragraph 14. The alpha-amylase variant according to any one of preceding paragraphs, wherein said alpha-amylase is derived from a Bacillus subtilis, B. licheniformis, Bacillus sp. KSM-K38, B. lentos, B. brevis, B. Stearothermophilus, B. alkalophilus, B. amyloliquefaciens, B. coagulans, B. circulans, B. lautus, B. thuringiensis, Streptomyces lividans, Bacillus halmapalus or S. murinus.
Paragraph 15. The alpha-amylase variant according to any one of preceding paragraphs, wherein said parent alpha-amylase is selected from the amino acid sequences set forth in SEQ ID NOs: 1, 2, 3, 4, 5, 6, 7, 8, 9, 10, 11, 12, 13 and 14, or any alpha-amylase has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to any of the amino acid sequences set forth in SEQ ID NOs: 1, 2 or 3 Paragraph 16. The alpha-amylase variant according to paragraphs, wherein said parent alpha-amylase comprises or consists of the amino acid sequence set forth in SEQ ID NO: 1, 2 and 3.
Paragraph 17. A polynucleotide encoding the alpha-amylase variant according to paragraphs 1-16.
Paragraph 18. A nucleic acid construct comprising the polynucleotide according to paragraph 17.
Paragraph 19. An expression vector comprising the polynucleotide according to paragraph 17.
Paragraph 20. A host cell comprising the polynucleotide according to paragraph 17, the nucleic acid construct according to paragraph 18, or the expression vector according to paragraph 19.
Paragraph 21. A method of producing an alpha-amylase variant comprising a. cultivating the host cell of paragraph 20 under conditions suitable for expression of said variant; and b. optionally recovering said variant.
Paragraph 22. A method of obtaining an alpha-amylase variant of a parent alpha-amylase by introducing a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering, and wherein the said variant has 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the SEQ ID No: 1, 2, 3, 4, 5, 6, 7, 8, 9, 10, 11, 12, 13 or 14.
Paragraph 23. A method of improving wash performance of alpha-amylase variant of a parent alpha-amylase having the amino acid sequence of SEQ ID NO: 1, or having at least 60% sequence identity thereto, said method comprising the steps of:
a) a substitution and/or deletion of two, three or four positions in the hybrid alpha-amylase said positions corresponding to position 180, 181, 182, and 183 using SEQ ID NO: 1 for numbering; and
b) a modification at one or more positions corresponding to positions: H1, H2, N3, G7, R26, A31, R37, T40, A41, N54, V56, A60, L63, K72, G73, K93, N94, Q98, V103, V104, M105, N106, A109, G110, A111, G113, T114, F116, V117, D118, D123, N126, N128, T131, S132, T134, Y135, Q136, I137, Q138, W140, K142, D144, P146, T151, Y152, F155, W159, Y160, T165, W167, E169, K172, L173, N174, I176, Y177, K178, A185, W188, E189, E193, N194, N196, F202, A203, L205, T224, N225, Y242, S243, F244, V252, T256, K258, E259, M260, F261, F266, N276, N279, K280, W283, S286, S302, G303, T315, H320, P321, M322, H323, W346, L354, E359, Q360, G361, Y362, V365, Y371, A383, E390, N394, F395, D405, I409, R414, H420, N422, T427, G455, K457, P458, N470, S472, V473, N474, G475, G476, using SEQ ID NO: 1 for numbering and wherein the said variant has 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the SEQ ID No: 1, 2, 3, 4, 5, 6, 7, 8, 9, 10, 11, 12, 13 or 14 and wherein said variant has alpha-amylase activity and improved wash performance compared to said parent polypeptide.
   Paragraph 24. A composition comprising the variant according to any one of paragraphs 1-16.
   Paragraph 25. The composition according to paragraph 24, further comprising at least one further active component.
   Paragraph 26. The composition according to paragraph 25, wherein active component is selected from the group consisting of surfactants, builders, flocculating aid, chelating agents, dye transfer inhibitors, enzyme stabilizers, enzyme inhibitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric hueing agents, anti-foaming agents, dispersants, processing aids, and/or pigments.
   Paragraph 27. The composition according to paragraph 26, wherein said active component is an enzyme.
   Paragraph 28. The composition according to any one of paragraphs 24-27, wherein the enzyme is selected from the group comprising proteases, amylases, lipases, cutinases, cellulases, endoglucanases, xyloglucanases, pectinases, pectin lyases, xanthanases, peroxidaes, haloperoxygenases, catalases and mannanases, or any mixture thereof.
   Paragraph 29. The composition according to any one of paragraphs 24-28, which is a detergent composition, such as a liquid detergent, solid detergent, gel detergent, powder detergent, tabs, pods, pouch and granule detergent.
   Paragraph 30. The composition according to any one of paragraphs 24-29, which is a liquid laundry or liquid dish wash composition, such as an Automatic Dish Wash (ADW) liquid detergent composition, or a powder laundry, such as a soap bar, or powder dish wash composition, such as an ADW unit dose detergent composition.
   Paragraph 31. Use of an alpha-amylase variant according to any one of paragraphs 1-16 in a cleaning process such as laundry or hard surface cleaning including dish wash and industrial cleaning.
   Paragraph 32. A hybrid polypeptide having alpha-amylase activity comprising an A-domain with at least 60% sequence identity with the A-domain of any of SEQ ID NOs: 1, 2, 3, 4 or 5, a B-domain with at least 60% sequence identity with the B-domain of any of SEQ ID NOs: 1, 2, 10, 11 or 12, and a C-domain with at least 60% sequence identity with the C-domain of any of SEQ ID NOs: 1, 2, 3, 4 or 5.
   Paragraph 33. The hybrid according to paragraph 32, wherein the A-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the A-domain of SEQ ID NO: 4.
   Paragraph 34. The hybrid according to paragraphs 32-33, wherein the A1-domain has at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the sequence starting at a position in the range of positions 1-5 and ending a position in the range of positions 94-114, e.g., starting at a position in the range of positions 1-3 and ending at a position in the range of positions 99-110 or starting at a position in the range of positions 1-3 and ending at a position in the range of positions 104-109, in particular positions 1-108 of SEQ ID NO: 4.
   Paragraph 35. The hybrid according to paragraphs 32-34, wherein the B-domain has at least 60% sequence identity, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the sequence starting at a position in the range of positions 93-113 and ending at a position in the range of positions 195-215, e.g., starting at a position in the range of positions 97-109 and ending at a position in the range of positions 199-211 or starting at a position in the range of positions 100-106 and ending at a position in the range of positions 202-208, in particular positions 109-205 of SEQ ID NO: 12.
   Paragraph 36. The hybrid according to paragraphs 32-35, wherein the C-domain has at least 60% sequence identity, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the C-domain of SEQ ID NO: 4.
   Paragraph 37. The hybrid according to paragraphs 32-36, wherein the A2 and C-domains have at least 60% sequence identity, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100% sequence identity with the sequence starting at a position in the range of positions 201-221 and ending at a position in the range of positions 478-514, e.g., starting at a position in the range of positions 203-211 and ending at a position in the range of positions 480-510 or starting at a position in the range of positions 205-216 and ending at a position in the range of positions 480-500, in particular positions 206-485 of SEQ ID NO: 4.
   Paragraph 38. The hybrid polypeptide according to any one of paragraphs 32-37, wherein the said hybrid polypeptide has improved property relative to said parent polypeptide of SEQ ID NO: 3.
   Paragraph 39. The hybrid polypeptide according to any one of paragraphs 32-38, wherein the improved property is increased wash performance as increased specific activity, particularly increased specific activity in Model A detergent composition.
   Paragraph 40. The hybrid polypeptide according to any one of paragraphs 32-39, wherein improved property is increased specific activity measured as Improvement Factor (IF) of >1.0 when compared to said parent polypeptide of SEQ ID NO: 3.
   Paragraph 41. The hybrid polypeptide according to any one of paragraphs 32-40, wherein said parent polypeptide has at least 60% such as 65%, such as at least 70%, such as at least 75%, such as at least 80%, such as at least 85%, such as at least 90%, such as at least 95%, such as at least 97%, such as at least 99%, but less than 100% sequence identity to any one of the SEQ ID NOs:1-14.
   Paragraph 42. The hybrid polypeptide according to any one of paragraphs 32-41, wherein said hybrid polypeptide has at least 60% such as 65%, such as at least 70%, such as at least 75%, such as at least 80%, such as at least 85%, such as at least 90%, such as at least 95%, such as at least 97%, such as at least 99%, but less than 100% sequence identity to any one of the SEQ ID NOs:1-14.
   Paragraph 43. The hybrid polypeptide according to any one of paragraphs 32-42 further comprises a deletion of one or more amino acids corresponding to positions 180, 181, 182 and 183 using SEQ ID NO: 1 for numbering.
   Paragraph 44. The hybrid polypeptide according to paragraph 43 comprises a deletion of two or more amino acids corresponding to positions 180, 181, 182 and 183 using SEQ ID NO: 1 for numbering.
   Paragraph 45. The hybrid polypeptide according to any of the preceding paragraph 43-44 comprises a deletion of the amino acids corresponding to positions 180 and 181 using SEQ ID NO: 1 for numbering.
   Paragraph 46. The hybrid polypeptide according to any of the preceding paragraphs 43-44 comprises a deletion of the amino acids corresponding to positions 180 and 182 using SEQ ID NO: 1 for numbering.
   Paragraph 47. The hybrid polypeptide according to any of the preceding paragraphs 43-44 comprises a deletion of the amino acids corresponding to positions 180 and 183 using SEQ ID NO: 1 for numbering.
   Paragraph 48. The hybrid polypeptide according to any of the preceding paragraphs 43-44 comprises a deletion of the amino acids corresponding to positions 181 and 182 using SEQ ID NO: 1 for numbering.
   Paragraph 49. The hybrid polypeptide according to any of the preceding paragraphs 43-44 comprises a deletion of the amino acids corresponding to positions 181 and 183 using SEQ ID NO: 1 for numbering.
   Paragraph 50. The hybrid polypeptide according to any of the preceding paragraphs 43-44 comprises a deletion of the amino acids corresponding to positions 182 and 183 using SEQ ID NO: 1 for numbering.
   Paragraph 51. The hybrid polypeptide according to any of the preceding paragraphs 43-44 comprising or consisting of SEQ ID NO: 1 or 2.
   Paragraph 52. The hybrid polypeptide according to any of the preceding paragraphs 32-51 which has at least one improved property relative to the parent polypeptide of SEQ ID NO: 3 wherein the improved property is selected from the group comprising detergent stability, increased specific activity, substrate specificity, thermal stability, pH-dependent activity, pH-dependent stability, oxidative stability, Ca2+ dependency and wash performance.
   Paragraph 53. The hybrid polypeptide according to any of the preceding paragraphs 32-52, wherein said alpha-amylase is derived from a Bacillus subtilis, B. licheniformis, Bacillus sp. KSM-K38, B. lentos, B. brevis, B. Stearothermophilus, B. alkalophilus, B. amyloliquefaciens, B. coagulans, B. circulans, B. lautus, B. thuringiensis, Streptomyces lividans, Bacillus halmapalus or S. murinus.
   Paragraph 54. A polynucleotide encoding the hybrid polypeptide according to any one of the preceding paragraphs 32-53.
   Paragraph 55. A nucleic acid construct comprising the polynucleotide according to paragraph 54.
   Paragraph 56. An expression vector comprising the polynucleotide according to paragraph 54.
   Paragraph 57. A host cell comprising the polynucleotide according to paragraph 54, the nucleic acid construct according to paragraph 55, or the expression vector according to paragraph 56.
   Paragraph 58. A method of improving the wash performance of an alpha amylase having at least 60% identity to the alpha amylase of SEQ ID NO: 4 or 5 said method comprising replacing the B domain of said alpha amylase with a B domain having amino acid 109-206 of SEQ ID NO: 12 or a sequence which has at least 60% identity hereto.
   Paragraph 59. A method of producing a hybrid polypeptide comprising: a. cultivating the host cell of paragraph 57 under conditions suitable for expression of said hybrid; and b. optionally recovering said hybrid.
   Paragraph 60. A cleaning composition comprising the hybrid according to any one of paragraphs 32-53.
   Paragraph 61. The composition according to paragraph 60, further comprising at least one further active component.
   Paragraph 62. The composition according to paragraph 61, wherein active component is selected from the group consisting of surfactants, builders, flocculating aid, chelating agents, dye transfer inhibitors, enzyme stabilizers, enzyme inhibitors, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, structure elasticizing agents, fabric softeners, carriers, hydrotropes, builders and co-builders, fabric hueing agents, anti-foaming agents, dispersants, processing aids, and/or pigments.
   Paragraph 63. The composition according to paragraph 62, wherein said active component is an enzyme.
   Paragraph 64. The composition according to any one of paragraphs 60-63, wherein the enzyme is selected from the group comprising proteases, amylases, lipases, cutinases, cellulases, endoglucanases, xyloglucanases, pectinases, pectin lyases, xanthanases, peroxidaes, haloperoxygenases, catalases and mannanases, or any mixture thereof.
   Paragraph 65. The composition according to any one of paragraphs 60-64, wherein the composition is selected from a group comprising: liquid detergent, solid detergent, gel detergent, powder detergent, tabs, pods, pouch and granule detergent.
   Paragraph 66 The composition according to any one of paragraphs 60-65, which is a liquid laundry or liquid dish wash composition, such as an Automatic Dish Wash (ADW) liquid detergent composition, or a powder laundry, such as a soap bar, or powder dish wash composition, such as an ADW unit dose detergent composition.
   Paragraph 67. Use of a hybrid polypeptide according to any one of paragraphs 60-66 in a cleaning process such as laundry or hard surface cleaning including dish wash and industrial cleaning.

### Material and Methods

### Assays for Alpha-Amylase Activity

pNP-G7 Assay

The alpha-amylase activity may be determined by a method employing the G7-pNP substrate. G7-pNP which is an abbreviation for 4,6-ethylidene(G₇)-p-nitrophenyl(G₁)-α,D-maltoheptaoside, a blocked oligosaccharide which can be cleaved by an endo-amylase, such as an alpha-amylase. Following the cleavage, the alpha-Glucosidase included in the kit digest the hydrolysed substrate further to liberate a free PNP molecule which has a yellow color and thus can be measured by visible spectophometry at λ=405 nm (400-420 nm.). Kits containing G7-pNP substrate and alpha-Glucosidase is manufactured by Roche/Hitachi (cat. No. 11876473).

### Reagents:

The G7-pNP substrate from this kit contains 22 mM 4,6-ethylidene-G7-pNP and 52.4 mM HEPES (2-[4-(2-hydroxyethyl)-1-piperazinyl]-ethanesulfonic acid), pH 7.0).

The alpha-Glucosidase reagent contains 52.4 mM HEPES, 87 mM NaCl, 12.6 mM MgCl₂, 0.075 mM CaCl₂, ≥4 kU/L alpha-glucosidase).

The substrate working solution is made by mixing 1 mL of the alpha-Glucosidase reagent with 0.2 mL of the G7-pNP substrate. This substrate working solution is made immediately before use.

Dilution buffer: 50 mM MOPS, 0.05% (w/v) Triton X100 (polyethylene glycol p-(1,1,3,3-tetramethylbutyl)-phenyl ether (C₁₄H₂₂O(C₂H₄O)ₙ (n=9-10))), 1 mM CaCl2, pH8.0.

### Procedure:

The amylase sample to be analyzed is diluted in dilution buffer to ensure the pH in the diluted sample is 7. The assay is performed by transferring 20 μl diluted enzyme samples to 96 well microtiter plate and adding 80 μl substrate working solution. The solution is mixed and pre-incubated 1 minute at room temperature and absorption is measured every 20 sec. over 5 minutes at OD 405 nm.

The slope (absorbance per minute) of the time dependent absorption-curve is directly proportional to the specific activity (activity per mg enzyme) of the alpha-amylase in question under the given set of conditions. The amylase sample should be diluted to a level where the slope is below 0.4 absorbance units per minute.

### Phadebas Activity Assay:

The alpha-amylase activity can also be determined by a method using the Phadebas substrate (from for example Magle Life Sciences, Lund, Sweden). A Phadebas tablet includes interlinked starch polymers that are in the form of globular microspheres that are insoluble in water. A blue dye is covantly bound to these microspheres. The interlinked starch polymers in the microsphere are degraded at a speed that is proportional to the alpha-amylase activity. When the alpha-amylase degrades the starch polymers, the released blue dye is water soluble and concentration of dye can be determined by measuring absorbance at 620 nm. The concentration of blue is proportional to the alpha-amylase activity in the sample.

The amylase sample to be analysed is diluted in activity buffer with the desired pH. One substrate tablet is suspended in 5 mL activity buffer and mixed on magnetic stirrer. During mixing of substrate transfer 150 μl to microtiter plate (MTP) or PCR-MTP. Add 30 μl diluted amylase sample to 150 μl substrate and mix. Incubate for 15 minutes at 37° C. The reaction is stopped by adding 30 μl 1 M NaOH and mix. Centrifuge MTP for 5 minutes at 4000×g. Transfer 100 μl to new MTP and measure absorbance at 620 nm.
The amylase sample should be diluted so that the absorbance at 620 nm is between 0 and 2.2, and is within the linear range of the activity assay.

### Reducing Sugar Activity Assay:

The alpha-amylase activity can also be determined by reducing sugar assay with for example corn starch substrate. The number of reducing ends formed by the alpha-amylase hydrolysing the alpha-1,4-glycosidic linkages in starch is determined by reaction with p-Hydroxybenzoic acid hydrazide (PHBAH). After reaction with PHBAH the number of reducing ends can be measured by absorbance at 405 nm and the concentration of reducing ends is proportional to the alpha-amylase activity in the sample.

The corns starch substrate (3 mg/ml) is solubilised by cooking for 5 minutes in milliQ water and cooled down before assay. For the stop solution prepare a Ka-Na-tartrate/NaOH solution (K—Na-tartrate (Merck 8087) 50 g/l, NaOH 20 g/l) and prepare freshly the stop solution by adding p-Hydroxybenzoic acid hydrazide (PHBAH, Sigma H9882) to Ka-Na-tartrate/NaOH solution to 15 mg/ml.
In PCR-MTP 50 μl activity buffer is mixed with 50 μl substrate. Add 50 μl diluted enzyme and mix. Incubate at the desired temperature in PCR machine for 5 minutes. Reaction is stopped by adding 75 μl stop solution (Ka-Na-tartrate/NaOH/PHBAH). Incubate in PCR machine for 10 minutes at 95° C. Transfer 150 μl to new MTP and measure absorbance at 405 nm.
The amylase sample should be diluted so that the absorbance at 405 nm is between 0 and 2.2, and is within the linear range of the activity assay.

### EnzChek® Assay:

For the determination of residual amylase activity an EnzChek® Ultra Amylase Assay Kit (E33651, Invitrogen, La Jolla, Calif., USA) may be used.

The substrate is a corn starch derivative, DQ™ starch, which is corn starch labeled with BODIPY® FL dye to such a degree that fluorescence is quenched. One vial containing approx. 1 mg lyophilized substrate is dissolved in 100 microliters of 50 mM sodium acetate (pH 4.0). The vial is vortexed for 20 seconds and left at room temperature, in the dark, with occasional mixing until dissolved. Then 900 microliters of 100 mM acetate, 0.01% (w/v) TRITON® X100, 0.125 mM CaCl₂, pH 5.5 is added, vortexed thoroughly and stored at room temperature, in the dark until ready to use. The stock substrate working solution is prepared by diluting 10-fold in residual activity buffer (100 mM acetate, 0.01% (w/v) TRITON® X100, 0.125 mM CaCl₂, pH 5.5). Immediately after incubation the enzyme is diluted to a concentration of 10-20 ng enzyme protein/ml in 100 mM acetate, 0.01% (W/v) TRITON® X100, 0.125 mM CaCl₂, pH 5.5.

For the assay, 25 microliters of the substrate working solution is mixed for 10 second with 25 microliters of the diluted enzyme in a black 384 well microtiter plate. The fluorescence intensity is measured (excitation: 485 nm, emission: 555 nm) once every minute for 15 minutes in each well at 25° C. and the Vₘₐₓ is calculated as the slope of the plot of fluorescence intensity against time. The plot should be linear and the residual activity assay has been adjusted so that the diluted reference enzyme solution is within the linear range of the activity assay.

### EXAMPLES

### Example 1: Construction of the Hybrid Polypeptide as Set Forth in SEQ ID NO: 1

Construction of a hybrid between the A and C domain from a polypeptide having amino acid no 1 to amino acid no 108 and amino acid no 207 to amino acid no 485 of amino acid sequence as set forth in SEQ ID NO: 3 (the first parent polypeptide) and the B domain from a polypeptide having amino acid no 109 to amino acid no 206 of amino acid sequence set forth SEQ ID NO: 11 (the second parent polypeptide).

Based on 3D structural alignment of the two amylases, amino acid no 1 to amino acid no 108 and amino acid no 207 to amino acid no 485 of the first parent polypeptide are defined as domain A and C and amino acid 109 to amino acid no. 206 of the second parent polypeptide are defined as the B domain. Synthetic DNA fragments coding for part of the A-C domain from the first parent polypeptide and the B domain from the second parent polypeptide were designed and purchased from an external vendor.

The resulting amylase consisting of the A and C domain from the first parent polypeptide and the B domain from the second parent polypeptide is the polypeptide as set forth in SEQ ID NO: 1. Further, the resulting amylase having the deletions of R180*+S181* is the polypeptide as set forth in SEQ ID NO: 2.

### Example 2: Specific Activity of the Hybrid Polypeptide

In order to determine whether the hybrid polypeptide generated as described in Example 1 has a maintained or even improved activity, the hybrid polypeptide was evaluated by the micro-swatch assay. The following detergent composition was prepared

TABLE 1
Model detergent A (percentages given in w/w)

| | Content of | % active |
|---|---|---|
| | compound | component |
| Compound | (% w/w) | (% w/w) |
| LAS | 12.00 | 11.60 |
| AEOS, SLES | 17.63 | 4.90 |
| soy fatty acid | 2.75 | 2.48 |
| coco fatty acid | 2.75 | 2.80 |
| AEO | 11.00 | 11.00 |
| Sodium hydroxide | 1.75 | 1.80 |
| Ethanol/propan-2-ol | 3.00 | 2.70/0.30 |
| MPG | 6.00 | 6.00 |
| Glycerol | 1.71 | 1.70 |
| TEA | 3.33 | 3.30 |
| Sodium formate | 1.00 | 1.00 |
| Sodium citrate | 2.00 | 2.00 |
| DTMPA | 0.48 | 0.20 |
| PCA | 0.46 | 0.18 |
| Phenoxy ethanol | 0.50 | 0.50 |
| H₂O, ion exchanged | 33.64 | 33.64 |

Preparation of Model A Detergent (0.33%): 4:1 Molar Ratio of CaCl₂ and MgCl₂ Stock Solution with 6000 dH (Water Hardness). 125.8 g of CaCl₂.2H₂O was weighed into 1-liter bottle and to this 500 ml of type I water was added and stirred well. To this 43.8 g of MgCl₂.6H₂O was weighed and added and dissolved well and the final volume was made up to 1000 ml with type I water.
0.535 M Solution of NaHCO₃: 44.9 g of Sodium Hydrogen carbonate was dissolved in 100 ml of type I water.
Model A Detergent with a Water Hardness of 15 (15° dH): 3.335 g of Model A detergent was weighed and transferred into 1 litre bottle and to this 865 ml of type I water was added and mixed well. To this 7.5 ml of 0.535M NaHCO₃ was added, mixed well and made up the volume to 1 liter with type 1 water. To adjust the water hardness to 15° dH, 2.5 ml of 4:1 molar ratio of CaCl₂.2H₂O and MgCl₂.6H₂O stock solution with 6000° dH was added and the mixture was stirred for 15 min.

### Experimental Procedure

### Preparation of the Mother Plates:

Colonies were picked from the transformed plate by colony picker (KBiosystems) and inoculated in 96-well culture plate comprising TBGly media for growth. The cultures were grown for 3 days at 37° C. and the supernatant was recovered from the plates by centrifugation.

### Preparation of the Substrate Plates:

CS-27, 5-meter swatches were obtained from Center for Test materials (CFT, The Netherlands) and were cut into 6 mm micro-swatches using swatch puncher. One or two swatches were placed in each well of 96-well Nunc plates to which 180 μl of Model A detergent (0.33%) was added. The culture supernatants or purified protein were diluted to 0.1 ppm or 0.4 ppm with 100 mM MOPS, pH 7 with 0.1 mM CaCl2, 0.01% Triton-X. To the plates containing the swatches and Model A detergent, 20 ul of diluted enzyme samples were added to get final concentration of 0.01 or 0.04 ppm and incubated at 25° C., 800 RPM for 10 min. 40 ul of the reaction mixture was transferred to another 96-well plate and in order to digest all the released oligosaccharides to glucose, 10 ul of 0.1 mg/ml of Amyloglucosidase (AMG) was added and incubated for 5 min at room temperature. AMG was purified from commercial product AMG 300L (Novozymes). The reducing ends were detected by p-hydroxybenzoic acid hydrazide (PAHBAH) method (Lever M Anal Biochem. 81:21-7). To prepare 1.5% PAHBAH reagent, 1.5 g of p-hydroxybenzoic acid hydrazide (PAHBAH) was dissolved in the buffer containing 1.0 M Na2CO3, 0.17 M potassium sodium tartrate and 5 mM Bi(NO3)3.5H2O. 50 ul AMG digest was added 50 ul of 1.5% PAHBAH reagent and heated at 95° C. for 10 mins. Absorbance of the resultant mixture was measured at 405 nm which is proportional to the enzyme activity. In case of culture supernatants, the concentration of expressed enzyme was determined by ELISA using specific antibodies. The specific activity was calculated by taking the ratio of the activity by protein concentration and the hit was identified as anything higher than the specific activity of the parent polypeptide (alpha-amylase).
The Improvement Factor (IF) was calculated as:

Improvement Factor (IF)=[Specific Activity of Hybrid polypeptide]/[Specific Activity of parent polypeptide (alpha-amylase)]

Specific activity of Hybrid Polypeptide=15.70
Specific activity of Parent Polypeptide=11.41

Improvement Factor (IF)=15.70/11.41=1.4.

Therefore, the IF of SEQ ID NO: 2 (hybrid polypeptide) is 1.4 and IF of SEQ ID NO: 3 (parent polypeptide) is 1.0.

### Example 3: Generation of Variants According to the Invention

The amylase variants of SEQ ID NO: 1 were prepared by standard procedures, in brief: Introducing random and/or site-directed mutations into the gene, transforming Bacillus subtilis host cells with the mutated genes, fermenting the transformed host cells (e.g. as described in Example 1 of WO 2004/111220). The alpha-amylase variants were produced recombinantly in Bacillus subtilis in a similar manner. A library of variants was generated where different combinations of alterations in positions were made. The generated variants are listed in the following examples.

### Example 4: Specific Activity of Variants

In order to determine whether the variants generated as described in Example 3 have a maintained or even improved activity, the variants were evaluated by the micro-swatch assay as described in Example 2. The specific activity was calculated by taking the ratio of the activity by protein concentration and the variants were identified as anything higher than the specific activity of the alpha-amylase of SEQ ID NO: 3 or SEQ ID NO: 2.

TABLE 2
The specific activity of the variants according to the invention obtained by the method described
above are the following (amino acid substitutions refer to SEQ ID NO: 1)

| | IF | IF |
|---|---|---|
| | (SEQ | (SEQ |
| | ID | ID |
| Modifications in SEQ ID NO: 1 | NO: 3) | NO: 2) |
| Parent polypeptide (alpha-amylase) SEQ ID NO: 3 | 1.0 | — |
| Hybrid polypeptide (alpha-amylase) SEQ ID No: 2 | — | 1.4 |
| H1* + G7A + R180* + S181* + Y242F + E259G + N279S + G303R + E390A + G475K | 7.0 | 5.1 |
| H1* + G7A + S181* + T182* + Y242F + E259G + N279S + G303R + E390A + G475K | 3.9 | 2.9 |
| H1* + G7A + R180* + S181* + T182S + Y242F + E259G + N279S + G303R + E390A + | 6.1 | 4.4 |
| G475K | | |
| H1* + G7A + R180* + T182L + G183* + Y242F + E259G + N279S + G303R + E390A + | 3.7 | 2.7 |
| G475K | | |
| H1* + G7A + S181L + T182* + G183* + Y242F + E259G + N279S + G303R + E390A + | 5.0 | 3.6 |
| G475K | | |
| H1* + G7A + R180S + T182* + G183* + Y242F + E259G + N279S + G303R + E390A + | 3.2 | 2.3 |
| G475K | | |
| H1* + G7A + A60P + R180* + S181* + Y242F + E259G + N279S + G303R + E390A + | 3.8 | 2.8 |
| G475K | | |
| H1* + G7A + M105L + R180* + S181* + Y242F + E259G + N279S + G303R + E390A + | 4.4 | 3.2 |
| G475K | | |
| H1* + G7A + M105I + R180* + S181* + Y242F + E259G + N279S + G303R + E390A + | 3.9 | 2.9 |
| G475K | | |
| H1* + G7A + R180* + S181* + T182D + Y242F + E259G + N279S + G303R + E390A + | 6.2 | 4.5 |
| G475K | | |
| H1* + G7A + R180* + S181* + T182E + Y242F + E259G + N279S + G303R + E390A + | 5.7 | 4.1 |
| G475K | | |
| H1* + G7A + R180* + S181* + T182G + Y242F + E259G + N279S + G303R + E390A + | 5.7 | 4.2 |
| G475K | | |
| H1* + G7A + R180* + S181* + T182M + Y242F + E259G + N279S + G303R + E390A + | 4.9 | 3.5 |
| G475K | | |
| H1* + G7A + R180* + S181* + A185K + Y242F + E259G + N279S + G303R + E390A + | 4.7 | 3.4 |
| G475K | | |
| H1* + G7A + R180* + S181* + A185D + Y242F + E259G + N279S + G303R + E390A + | 5.0 | 3.6 |
| G475K | | |
| H1* + G7A + R180* + S181* + A185Q + Y242F + E259G + N279S + G303R + E390A + | 5.7 | 4.1 |
| G475K | | |
| H1* + G7A + R180* + S181* + A185N + Y242F + E259G + N279S + G303R + E390A + | 5.1 | 3.7 |
| G475K | | |
| H1* + G7A + R180* + S181* + A185R + Y242F + E259G + N279S + G303R + E390A + | 4.3 | 3.1 |
| G475K | | |
| H1* + G7A + R180* + S181* + A185E + Y242F + E259G + N279S + G303R + E390A + | 6.1 | 4.5 |
| G475K | | |
| H1* + G7A + R180* + S181* + E189P + Y242F + E259G + N279S + G303R + E390A + | 3.7 | 2.7 |
| G475K | | |
| H1* + G7A + R180* + S181* + L205Y + Y242F + E259G + N279S + G303R + E390A + | 4.3 | 3.1 |
| G475K | | |
| H1* + G7A + R180* + S181* + L205F + Y242F + E259G + N279S + G303R + E390A + | 4.6 | 3.4 |
| G475K | | |
| H1* + G7A + R180* + S181* + Y242F + S243Q + E259G + N279S + G303R + E390A + | 5.1 | 3.7 |
| G475K | | |
| H1* + G7A + R180* + S181* + Y242F + F244L + E259G + N279S + G303R + E390A + | 4.8 | 3.5 |
| G475K | | |
| H1* + G7A + R180* + S181* + Y242F + F244I + E259G + N279S + G303R + E390A + | 4.4 | 3.2 |
| G475K | | |
| H1* + G7A + R180* + S181* + Y242F + E259G + F266Y + N279S + G303R + E390A + | 5.9 | 4.3 |
| G475K | | |
| H1* + G7A + R180* + S181* + N194F + Y242F + E259G + N279S + G303R + E390A + | 6.5 | 4.7 |
| G475K | | |
| H1* + G7A + R180* + S181* + N196F + Y242F + E259G + N279S + G303R + E390A + | 5.2 | 3.8 |
| G475K | | |
| H1* + G7A + R180* + S181* + N194F + L205Y + Y242F + E259G + N279S + G303R + | 5.5 | 4.0 |
| E390A + G475K | | |
| H1* + G7A + R180* + S181* + N194F + L205F + Y242F + E259G + N279S + G303R + | 5.3 | 3.9 |
| E390A + G475K | | |
| H1* + G7A + R180* + S181* + N196F + L205Y + Y242F + E259G + N279S + G303R + | 4.6 | 3.4 |
| E390A + G475K | | |
| H1* + G7A + R180* + S181* + E189P + Y242F + E259G + N276F + N279S + G303R + | 3.8 | 2.7 |
| E390A + G475K | | |
| H1* + G7A + R180* + S181* + E189P + Y242F + E259G + N276Y + N279S + G303R + | 3.5 | 2.5 |
| E390A + G475K | | |
| H1* + G7A + R180* + S181* + E189P + Y242F + E259G + N276W + N279S + G303R + | 2.9 | 2.1 |
| E390A + G475K | | |
| H1* + G7A + R180* + S181* + E189P + Y242F + E259G + N279S + K280Y + G303R + | 7.1 | 5.1 |
| E390A + G475K | | |
| H1* + G7A + R180* + S181* + E189P + Y242F + E259G + N279S + K280W + G303R + | 5.3 | 3.9 |
| E390A + G475K | | |
| H1* + G7A + R180* + S181* + E189P + L205F + Y242F + E259G + N276F + N279S + | 3.5 | 2.5 |
| G303R + E390A + G475K | | |
| H1* + G7A + R180* + S181* + E189P + L205F + Y242F + E259G + N276Y + N279S + | 3.2 | 2.3 |
| G303R + E390A + G475K | | |
| H1* + G7A + R180* + S181* + E189P + L205F + Y242F + E259G + N276W + N279S + | 2.2 | 1.6 |
| G303R + E390A + G475K | | |
| H1* + G7A + R180* + S181* + E189P + L205F + Y242F + E259G + N279S + K280F + | 4.3 | 3.1 |
| G303R + E390A + G475K | | |
| H1* + G7A + R180* + S181* + E189P + L205F + Y242F + E259G + N279S + K280Y + | 4.2 | 3.1 |
| G303R + E390A + G475K | | |
| H1* + G7A + R180* + S181* + E189P + L205F + Y242F + E259G + N279S + K280W + | 4.2 | 3.0 |
| G303R + E390A + G475K | | |
| H1* + G7A + R180* + S181* + E189P + L205Y + Y242F + E259G + N276F + N279S + | 3.1 | 2.3 |
| G303R + E390A + G475K | | |
| H1* + G7A + R180* + S181* + E189P + L205Y + Y242F + E259G + N276Y + N279S + | 3.0 | 2.2 |
| G303R + E390A + G475K | | |
| H1* + G7A + R180* + S181* + E189P + L205Y + Y242F + E259G + N276W + N279S + | 2.1 | 1.6 |
| G303R + E390A + G475K | | |
| H1* + G7A + R180* + S181* + E189P + L205Y + Y242F + E259G + N279S + K280F + | 4.1 | 3.0 |
| G303R + E390A + G475K | | |
| H1* + G7A + R180* + S181* + E189P + L205Y + Y242F + E259G + N279S + K280Y + | 4.2 | 3.0 |
| G303R + E390A + G475K | | |
| H1* + G7A + R180* + S181* + E189P + L205Y + Y242F + E259G + N279S + K280W + | 4.2 | 3.1 |
| G303R + E390A + G475K | | |
| H1* + G7A + G113S + F116W + D123N + S181* + T182* + Y242F + E259G + N279S + | 3.0 | 2.1 |
| G303R + E390A + G475K | | |
| H1* + G7A + F116L + D123N + N126D + T131I + S181* + T182* + F202Y + Y242F + | 4.6 | 3.4 |
| E259G + N279S + G303R + E390A + G475K | | |
| H1* + G7S + T40N + Q98L + R180* + S181* + Y242F + E259G + N279S + G303R + | 8.6 | 6.3 |
| Y362V + E390A + G475K | | |
| H1* + G7A + R180* + S181* + Y242F + E259G + N279S + G303R + M322G + E359W + | 6.4 | 4.6 |
| Q360W + E390A + G475K | | |
| H1* + G7A + R180* + S181* + Y242F + E259G + N279S + G303R + M322G + Q360W + | 8.0 | 5.8 |
| E390A + G475K | | |
| H1* + G7A + R180* + S181* + Y242F + E259G + N279S + G303R + E359W + Q360W + | 5.8 | 4.2 |
| E390A + G475K | | |
| H1* + G7S + R180* + S181* + Y242F + E259G + F261S + N279S + G303R + Y362V + | 7.5 | 5.4 |
| E390A + G475K | | |
| H1* + G7A + T40N + Q98L + R180* + S181* + Y242F + E259G + N279S + G303R + | 7.7 | 5.6 |
| E390A + G475K | | |
| H1* + G7S + T40N + Q98L + R180* + S181* + Y242F + E259G + N279S + G303R + | 7.8 | 5.7 |
| E390A + G475K | | |
| H1* + G7A + R180* + S181* + Y242F + E259G + F261S + N279S + S286K + G303R + | 8.0 | 5.8 |
| Y362V + E390A + G475K | | |
| H1* + G7A + R180* + S181* + Y242F + E259G + N279S + W283N + S286K + G303R + | 6.1 | 4.5 |
| H320T + H323A + E390A + G475K | | |
| H1* + G7A + R180* + S181* + Y242F + E259G + N279S + G303R + P321H + M322Y + | 5.8 | 4.2 |
| Q360Y + E390A + G475K | | |
| H1* + G7A + D123N + I176V + R180* + S181* + Y242F + E259G + N279S + G303R + | 6.6 | 4.8 |
| E390A + G475K | | |
| H1* + G7A + G113A + F116W + D123N + T131V + Q136K + E169Q + K172Q + R180* + | 4.3 | 3.1 |
| S181* + Y242F + E259G + N279S + G303R + E390A + G475K | | |
| H1* + G7A + G113S + F116W + D123N + R180* + S181* + Y242F + E259G + N279S + | 6.7 | 4.8 |
| G303R + E390A + G475K | | |
| H1* + G7A + F116L + D123N + N126D + T131I + R180* + S181* + F202Y + Y242F + | 8.6 | 6.3 |
| E259G + N279S + G303R + E390A + G475K | | |
| H1* + G7A + N106D + A109G + F116W + D123N + N126D + T131I + T165V + N174S + | 6.7 | 4.9 |
| R180* + S181* + T182I + F202Y + Y242F + E259G + N279S + G303R + E390A + | | |
| G475K | | |
| H1* + G7S + T40N + Q98L + R180* + S181* + Y242F + E259G + N279S + G303R + | 7.8 | 5.6 |
| H320T + E390A + G475K | | |
| H1* + G7S + T40N + Q98L + R180* + S181* + Y242F + E259G + N279S + G303R + | 7.4 | 5.4 |
| Y362V + E390A + G475K | | |
| H1* + G7A + R180* + S181* + Y242F + E259G + N279S + G303R + M322G + E390A + | 6.0 | 4.4 |
| G475K | | |
| H1* + G7A + R180* + S181* + Y242F + E259G + N279S + G303R + M322G + Q360W + | 7.3 | 5.3 |
| E390A + G475K | | |
| H1* + G7A + T40N + R180* + S181* + Y242F + E259G + N279S + G303R + E390A + | 7.2 | 5.2 |
| G475K | | |
| H1* + G7S + Q98L + R180* + S181* + Y242F + E259G + N279S + G303R + E390A + | 7.2 | 5.2 |
| G475K | | |
| H1* + G7S + R180* + S181* + Y242F + E259G + F261K + N279S + G303R + E390A + | 7.5 | 5.4 |
| G475K | | |
| H1* + G7S + R180* + S181* + Y242F + E259G + N279S + G303R + Y362V + E390A + | 6.4 | 4.6 |
| G475K | | |
| H1* + G7A + T40N + Q98L + R180* + S181* + Y242F + E259G + N279S + G303R + | 7.3 | 5.3 |
| E390A + G475K | | |
| H1* + G7A + R180* + S181* + Y242F + E259G + N279S + G303R + Y362V + E390A + | 5.9 | 4.3 |
| G475K | | |
| H1* + G7S + R180* + S181* + Y242F + E259G + F261S + N279S + G303R + E390A + | 6.0 | 4.3 |
| G475K | | |
| H1* + G7A + I137F + R180* + S181* + Y242F + E259G + F261S + N279S + G303R + | 5.7 | 4.1 |
| Y362V + E390A + G475K | | |
| H1* + G7A + R180* + S181* + Y242F + E259G + N279S + S286K + G303R + H323A + | 5.6 | 4.1 |
| E390A + G475K | | |
| H1* + G7A + R180* + S181* + Y242F + E259G + N279S + G303R + H320T + H323A + | 5.5 | 4.0 |
| E390A + G475K | | |
| H1* + G7A + R180* + S181* + Y242F + E259G + N279S + G303R + P321H + M322Y + | 5.0 | 3.6 |
| E390A + G475K | | |
| H1* + G7A + R180* + S181* + Y242F + E259G + N279S + G303R + P321H + Q360Y + | 5.2 | 3.8 |
| E390A + G475K | | |
| H1* + G7A + R180* + S181* + Y242F + E259G + N279S + G303R + M322Y + Q360Y + | 6.0 | 4.3 |
| E390A + G475K | | |
| H1* + G7S + T40N + R180* + S181* + Y242F + E259G + N279S + G303R + Y362V + | 6.2 | 4.5 |
| E390A + G475K | | |
| H1* + G7S + R180* + S181* + Y242F + E259G + F261S + N279S + G303R + Y362V + | 8.1 | 5.9 |
| E390A + G475K | | |
| H1* + G7A + R180* + S181* + Y242F + E259G + F261S + N279S + S286K + G303R + | 6.8 | 4.9 |
| Y362V + E390A + G475K | | |
| H1* + G7A + R180* + S181* + Y242F + E259G + N279S + S286L + G303R + E390A + | 6.5 | 4.7 |
| G475K | | |
| H1* + G7A + G110K + R180* + S181* + Y242F + E259G + N279S + G303R + E390A + | 7.7 | 5.6 |
| G475K | | |
| H1* + G7A + W140S + R180* + S181* + Y242F + E259G + N279S + G303R + E390A + | 1.9 | 1.4 |
| G475K | | |
| H1* + G7A + W167P + R180* + S181* + Y242F + E259G + N279S + G303R + E390A + | 3.5 | 2.5 |
| G475K | | |
| H1* + G7A + R180* + S181* + Y242F + E259G + N279S + G303R + H323A + E390A + | 6.6 | 4.8 |
| G475K | | |
| H1* + G7A + R180* + S181* + Y242F + E259G + N279S + W283N + G303R + H320T + | 6.3 | 4.5 |
| H323A + E390A + G475K | | |
| H1* + G7A + R180* + S181* + Y242F + E259G + N279S + G303R + H320T + E390A + | 7.0 | 5.1 |
| G475K | | |
| H1* + G7A + R180* + S181* + Y242F + E259G + N279S + G303R + P321H + E390A + | 6.6 | 4.8 |
| G475K | | |
| H1* + G7A + R180* + S181* + Y242F + E259G + N279S + G303R + M322Y + E390A + | 6.3 | 4.6 |
| G475K | | |
| H1* + G7A + R180* + S181* + Y242F + E259G + N279S + G303R + Q360Y + E390A + | 6.6 | 4.8 |
| G475K | | |
| H1* + G7S + T40N + Q98L + R180* + S181* + Y242F + E259G + N279S + G303R + | 6.5 | 4.7 |
| E390A + G475K | | |
| H1* + G7A + Q98L + R180* + S181* + Y242F + E259G + F261K + N279S + G303R + | 6.9 | 5.0 |
| Y362V + E390A + G475K | | |
| H1* + G7A + Q98L + R180* + S181* + Y242F + E259G + N279S + G303R + E390A + | 7.0 | 5.1 |
| G475K | | |
| H1* + G7A + R180* + S181* + Y242F + E259G + F261S + N279S + G303R + Y362V + | 6.4 | 4.6 |
| E390A + G475K | | |
| H1* + G7A + R180* + S181* + Y242F + E259G + N279S + S286L + G303R + H320T + | 6.1 | 4.4 |
| E390A + G475K | | |
| H1* + G7A + R180* + S181* + Y242F + E259G + F261S + N279S + G303R + E390A + | 6.5 | 4.7 |
| G475K | | |
| H1* + G7A + W140Y + R180* + S181* + T182D + N194F + L205Y + Y242F + E259G + | 6.7 | 4.9 |
| N279S + G303R + E390A + G475K | | |
| H1* + G7A + W140Y + R180* + S181* + T182D + N194F + L205V + Y242F + E259G + | 8.1 | 5.9 |
| N279S + G303R + E390A + G475K | | |
| H1* + G7A + D118T + W140Y + R180* + S181* + T182D + N194F + L205Y + Y242F + | 7.6 | 5.5 |
| E259G + N279S + G303R + E390A + G475K | | |
| H1* + G7A + F116N + W140Y + R180* + S181* + T182D + N194F + L205Y + Y242F + | 7.0 | 5.1 |
| E259G + N279S + G303R + E390A + G475K | | |
| H1* + G7A + W140Y + R180* + S181* + E189P + N194F + L205Y + Y242F + E259G + | 8.1 | 5.9 |
| N279S + K280Y + G303R + E390A + G475K | | |
| H1* + G7A + D118T + W140Y + R180* + S181* + E189P + N194F + L205Y + Y242F + | 4.4 | 3.2 |
| E259G + N279S + K280Y + G303R + E390A + G475K | | |
| H1* + G7A + F116N + W140Y + R180* + S181* + E189P + N194F + L205Y + Y242F + | 8.0 | 5.8 |
| E259G + N279S + K280Y + G303R + E390A + G475K | | |
| H1* + G7A + W140Y + R180* + S181* + T182D + E189P + N194F + L205Y + Y242F + | 6.6 | 4.8 |
| E259G + N279S + G303R + E390A + G475K | | |
| H1* + G7A + T134D + W1 40Y + R180* + S181* + E189P + N194F + L205V + Y242F + | 6.8 | 5.0 |
| E259G + N279S + G303R + E390A + G475K | | |
| H1* + G7A + F116N + W140Y + R180* + S181* + T182D + E189P + N194F + L205Y + | 6.7 | 4.9 |
| Y242F + E259G + N279S + G303R + E390A + G475K | | |
| H1* + G7A + W140Y + R180* + S181* + T182S + N194F + L205Y + Y242F + E259G + | 7.5 | 5.5 |
| N279S + G303R + E390A + G475K | | |
| H1* + G7A + W140Y + R180* + S181* + T182S + N194F + L205V + Y242F + E259G + | 8.7 | 6.3 |
| N279S + G303R + E390A + G475K | | |
| H1* + G7A + D118T + W140Y + R180* + S181* + T182S + N194F + L205Y + Y242F + | 7.1 | 5.2 |
| E259G + N279S + G303R + E390A + G475K | | |
| H1* + G7A + F116N + W140Y + R180* + S181* + T182S + N194F + L205Y + Y242F + | 7.9 | 5.8 |
| E259G + N279S + G303R + E390A + G475K | | |
| H1* + G7A + W140Y + R180* + S181* + T182S + E189P + N194F + L205Y + Y242F + | 6.5 | 4.7 |
| E259G + N279S + G303R + E390A + G475K | | |
| H1* + G7A + W140Y + R180* + S181* + T182S + E189P + N194F + L205V + Y242F + | 7.0 | 5.1 |
| E259G + N279S + G303R + E390A + G475K | | |
| H1* + G7A + F116N + W140Y + R180* + S181* + T182S + E189P + N194F + L205Y + | 4.9 | 3.6 |
| Y242F + E259G + N279S + K280Y + G303R + E390A + G475K | | |
| H1* + G7A + D123N + W140Y + R180* + S181* + N194F + L205V + Y242F + E259G + | 6.7 | 4.9 |
| N279S + G303R + E390A + G475K | | |
| H1* + G7A + D123N + W140Y + I176V + R180* + S181* + N194F + L205V + Y242F + | 6.7 | 4.9 |
| E259G + N279S + G303R + E390A + G475K | | |
| H1* + G7A + G113S + F116W + D123N + W140Y + R180* + S181* + N194F + L205V + | 5.6 | 4.1 |
| Y242F + E259G + N279S + G303R + E390A + G475K | | |
| H1* + G7A + F116L + D123N + N126D + T131I + W140Y + R180* + S181* + N194F + | 7.1 | 5.2 |
| F202Y + L205V + Y242F + E259G + N279S + G303R + E390A + G475K | | |
| H1* + G7A + N106D + A109G + W140Y + R180* + S181* + N194F + F202Y + | 5.3 | 3.8 |
| L205V + Y242F + E259G + N279S + G303R + E390A + G475K | | |
| H1* + G7A + D123N + W140Y + R180* + S181* + N194F + L205Y + Y242F + E259G + | 6.0 | 4.4 |
| N279S + G303R + E390A + G475K | | |
| H1* + G7A + D123N + W140Y + I176V + R180* + S181* + N194F + L205Y + Y242F + | 6.1 | 4.4 |
| E259G + N279S + G303R + E390A + G475K | | |
| H1* + G7A + G113S + F116W + D123N + W140Y + R180* + S181* + N194F + L205Y + | 4.5 | 3.3 |
| Y242F + E259G + N279S + G303R + E390A + G475K | | |
| H1* + G7A + F116L + D123N + N126D + T131I + W140Y + R180* + S181* + N194F + | 6.6 | 4.8 |
| F202Y + L205Y + Y242F + E259G + N279S + G303R + E390A + G475K | | |
| H1* + G7A + Q98L + G110K + P146S + R180* + S181* + Y242F + E259G + N279S + | 5.8 | 4.2 |
| G303R + A383D + E390A + G475K | | |
| H1* + N3S + G7A + Q98L + G110K + R180* + S181* + Y242F + V252I + E259G + | 5.4 | 3.9 |
| N279S + G303R + E390A + G475K | | |
| H1* + G7H + Q98L + R180* + S181* + Y242F + E259G + N279S + G303R + A383D + | 4.9 | 3.5 |
| E390A + D405G + G475K | | |
| H1* + G7H + Q98L + G 110K + R180* + S181* + Y242F + E259G + N279S + G303R + | 5.3 | 3.9 |
| E390A + G475K | | |
| H1* + G7A + Q98L + G110K + S132D + Y135E + R180* + S181* + Y242F + E259G + | 5.6 | 4.1 |
| N279S + G303R + T315A + E390A + G475K | | |
| H1* + G7H + G110K + R180* + S181* + Y242F + E259G + N279S + G303R + E390A + | 5.9 | 4.3 |
| G475K | | |
| H1* + A60V + Q98L + W140Y + R180* + S181* + N194F + L205Y + Y242F + | 6.1 | 4.4 |
| E259G + N279S + G303R + A383D + E390A + G475K | | |
| H1* + G7A + W140Y + T151A + R180* + S181* + N194F + L205Y + Y242F + K258V + | 7.1 | 5.2 |
| E259V + N279S + G303R + E390A + G475K | | |
| H1* + N3S + G7A + N94D + Q98L + W140Y + R180* + S181* + N194F + L205Y + | 6.0 | 4.3 |
| Y242F + V252V + E259G + N279S + G303R + E390A + G475K | | |
| H1* + G7A + Q98L + G110K + R180* + S181* + Y242F + T256A + E259G + N279S + | 5.7 | 4.1 |
| G303R + E390A + G475K | | |
| H1* + G7A + Q98L + G110K + R180* + S181* + Y242F + E259G + N279S + G303R + | 5.7 | 4.1 |
| A383D + E390A + G475K | | |
| H1* + N3S + G7A + N94D + Q98L + G110K + R180* + S181* + Y242F + V252I + | 5.9 | 4.3 |
| E259G + N279S + G303R + E390A + G475K | | |
| H1* + G7A + Q98L + G110K + R180* + S181* + Y242F + E259G + N279S + G303R+ | 5.0 | 3.7 |
| E390A + G475K | | |
| H1* + G7H + Q98L + G110K + R180* + S181* + Y242F + E259G + N279S + G303R + | 5.3 | 3.9 |
| E390A + G475K | | |
| H1* + G7A + W140Y + T151A + R180* + S181* + N194F + L205Y + Y242F + K258V + | 6.0 | 4.4 |
| E259V + N279S + G303R + E390A + G475K | | |
| H1* + G7A + Q98L + G110K + R180* + S181* + Y242F + E259G + N279S + G303R + | 5.9 | 4.3 |
| E390A + G475K | | |
| H1* + G7A + R180* + S181* + Y242F + E259G + N279S + S286L + G303R + Y362V + | 5.3 | 3.8 |
| E390A + G475K | | |
| H1* + G7A + R180* + S181* + Y242F + E259G + N279S + W283N + S286K + G303R + | 4.8 | 3.5 |
| E390A + G475K | | |
| H1* + G7A + R180* + S181* + Y242F + E259G + N279S + W283N + G303R + H320T + | 5.7 | 4.1 |
| E390A + G475K | | |
| H1* + G7A + R180* + S181* + Y242F + E259G + N279S + W283N + G303R + H323A + | 5.2 | 3.8 |
| E390A + G475K | | |
| H1* + G7A + R180* + S181* + Y242F + E259G + N279S + S286K + G303R + H320T + | 4.7 | 3.5 |
| E390A + G475K | | |
| H1* + G7A + R180* + S181* + Y242F + E259G + N279S + S286K + G303R + Y362V + | 5.7 | 4.2 |
| E390A + G475K | | |
| H1* + G7S + R180* + S181* + Y242F + E259G + N279S + G303R + E390A + G475K | 5.4 | 4.0 |
| H1* + G7A + Q98L + R180* + S181* + Y242F + E259G + N279S + G303R + E390A + | 5.4 | 3.9 |
| G475K | | |
| H1* + G7A + R180* + S181* + Y242F + E259G + F261K + N279S + G303R + E390A + | 5.4 | 3.9 |
| G475K | | |
| H1* + G7A + R180* + S181* + Y242F + E259G + N279S + G303R + M322G + E359W + | 4.5 | 3.3 |
| E390A + G475K | | |
| H1* + G7A + F116N + W140Y + R180* + S181* + T182D + E189P + N194F + L205Y + | 5.6 | 4.0 |
| Y242F + E259G + N279S + K280Y + G303R + E390A + G475K | | |
| H1* + G7A + F116N + W140Y + R180* + S181* + T182D + E189P + N194F + Y242F + | 6.4 | 4.6 |
| E259G + N279S + K280Y + G303R + E390A + G475K | | |
| H1* + G7A + F116N + W140Y + R180* + S181* + T182S + E189P + N194F + Y242F + | 6.4 | 4.6 |
| E259G + N279S + K280Y + G303R + E390A + G475K | | |
| H1* + G7A + F116N + W140Y + R180* + S181* + T182D + N194F + L205Y + Y242F + | 5.7 | 4.1 |
| E259G + F266Y + N279S + G303R + E390A + G475K | | |
| H1* + G7A + F116N + W140Y + R180* + S181* + A185Q + N194F + L205Y + Y242F + | 6.1 | 4.4 |
| E259G + F266Y + N279S + G303R + E390A + G475K | | |
| H1* + G7A + F116N + W140Y + R180* + S181* + T182D + A185Q + N194F + L205Y + | 5.6 | 4.1 |
| Y242F + E259G + F266Y + N279S + G303R + E390A + G475K | | |
| H1* + G7A + F116N + W140Y + R180* + S181* + N194F + L205Y + Y242F + | 6.4 | 4.6 |
| E259G + F266Y + N279S + G303R + E390A + G475K | | |
| H1* + G7A + F116N + W140Y + R180* + S181* + A185Q + N194F + L205Y + | 6.6 | 4.8 |
| Y242F + E259G + N279S + G303R + E390A + G475K | | |
| H1* + G7A + F116N + W140Y + R180* + S181* + T182D + A185Q + N194F + L205Y + | 6.0 | 4.3 |
| Y242F + E259G + N279S + G303R + E390A + G475K | | |
| H1* + G7A + D118T + W140Y + R180* + S181* + E189P + N194F + Y242F + | 6.1 | 4.4 |
| E259G + N279S + K280Y + G303R + E390A + G475K | | |
| H1* + G7A + D118T + W140Y + R180* + S181* + T182S + E189P + N194F + L205Y + | 5.5 | 4.0 |
| Y242F + E259G + N279S + K280Y + G303R + E390A + G475K | | |
| H1* + G7A + D118T + W140Y + R180* + S181* + T182D + N194F + L205Y + | 5.8 | 4.2 |
| Y242F + E259G + F266Y + N279S + G303R + E390A + G475K | | |
| H1* + G7A + D118T + W140Y + R180* + S181* + A185Q + N194F + L205Y + | 5.2 | 3.8 |
| Y242F + E259G + F266Y + N279S + G303R + E390A + G475K | | |
| H1* + G7A + D118T + W140Y + R180* + S181* + T182D + A185Q + N194F + L205Y + | 5.5 | 4.0 |
| Y242F + E259G + F266Y + N279S + G303R + E390A + G475K | | |
| H1* + G7A + D118T + W140Y + R180* + S181* + N194F + L205Y + Y242F + | 5.4 | 3.9 |
| E259G + F266Y + N279S + G303R + E390A + G475K | | |
| H1* + G7A + D118T + W140Y + R180* + S181* + A185Q + N194F + L205Y + | 5.5 | 4.0 |
| Y242F + E259G + N279S + G303R + E390A + G475K | | |
| H1* + G7A + D118T + W140Y + R180* + S181* + T182D + A185Q + N194F + | 6.2 | 4.5 |
| L205Y + Y242F + E259G + N279S + G303R + E390A + G475K | | |
| H1* + G7A + W140Y + R180* + S181* + E189P + N194F + L205V + Y242F + | 6.4 | 4.6 |
| E259G + N279S + K280Y + G303R + E390A + G475K | | |
| H1* + G7A + W140Y + R180* + S181* + T182D + E189P + N194F + L205V + | 6.6 | 4.8 |
| Y242F + E259G + N279S + K280Y + G303R + E390A + G475K | | |
| H1* + G7A + W140Y + R180* + S181* + T182D + E189P + N194F + Y242F + | 6.3 | 4.6 |
| E259G + N279S + K280Y + G303R + E390A + G475K | | |
| H1* + G7A + W140Y + R180* + S181* + T182S + E189P + N194F + Y242F + | 6.5 | 4.7 |
| E259G + N279S + K280Y + G303R + E390A + G475K | | |
| H1* + G7A + W140Y + R180* + S181* + T182D + N194F + L205V + Y242F + | 6.3 | 4.6 |
| E259G + F266Y + N279S + G303R + E390A + G475K | | |
| H1* + G7A + W140Y + R180* + S181* + A185Q + N194F + L205V + Y242F + | 6.7 | 4.9 |
| E259G + F266Y + N279S + G303R + E390A + G475K | | |
| H1* + G7A + W140Y + R180* + S181* + T182D + A185Q + N194F + L205V + | 5.8 | 4.2 |
| Y242F + E259G + F266Y + N279S + G303R + E390A + G475K | | |
| H1* + G7A + W140Y + R180* + S181* + N194F + L205V + Y242F + E259G + | 6.6 | 4.8 |
| F266Y + N279S + G303R + E390A + G475K | | |
| H1* + G7A + W140Y + R180* + S181* + A185Q + N194F + L205V + Y242F + | 6.5 | 4.7 |
| E259G + N279S + G303R + E390A + G475K | | |
| H1* + G7A + W140Y + R180* + S181* + T182D + A185Q + N194F + L205V + | 6.3 | 4.6 |
| Y242F + E259G + N279S + G303R + E390A + G475K | | |
| H1* + G7A + W140Y + R180* + S181* + T182D + E189P + N194F + L205Y + | 5.7 | 4.1 |
| Y242F + E259G + N279S + K280Y + G303R + E390A + G475K | | |
| H1* + G7A + W140Y + R180* + S181* + T182D + E189P + N194F + Y242F + | 6.0 | 4.4 |
| E259G + N279S + K280Y + G303R + E390A + G475K | | |
| H1* + G7A + W140Y + R180* + S181* + T182S + E189P + N194F + L205Y + Y242F + | 6.4 | 4.6 |
| E259G + N279S + K280Y + G303R + E390A + G475K | | |
| H1* + G7A + W140Y + R180* + S181* + T182S + E189P + N194F + Y242F + E259G + | 6.8 | 5.0 |
| N279S + K280Y + G303R + E390A + G475K | | |
| H1* + G7A + W140Y + R180* + S181* + T182D + N194F + L205Y + Y242F + E259G + | 5.6 | 4.1 |
| F266Y + N279S + G303R + E390A + G475K | | |
| H1* + G7A + W140Y + R180* + S181* + A185Q + N194F + L205Y + Y242F + E259G + | 6.2 | 4.5 |
| F266Y + N279S + G303R + E390A + G475K | | |
| H1* + G7A + W140Y + R180* + S181* + T182D + A185Q + N194F + L205Y + | 5.5 | 4.0 |
| Y242F + E259G + F266Y + N279S + G303R + E390A + G475K | | |
| H1* + G7A + W140Y + R180* + S181* + A185Q + N194F + L205Y + Y242F + | 5.9 | 4.3 |
| E259G + N279S + G303R + E390A + G475K | | |
| H1* + G7A + W140Y + R180* + S181* + T182D + A185Q + N194F + L205Y + | 5.3 | 3.8 |
| Y242F + E259G + N279S + G303R + E390A + G475K | | |
| H1* + G7A + F116N + W140Y + R180* + S181* + E189P + N194F + Y242F + E259G + | 6.4 | 4.7 |
| N279S + K280Y + G303R + E390A + G475K | | |
| H1* + G7A + D118T + W140Y + R180* + S181* + T182D + E189P + N194F + L205Y + | 5.3 | 3.8 |
| Y242F + E259G + N279S + K280Y + G303R + E390A + G475K | | |
| H1* + G7A + D118T + W140Y + R180* + S181* + T182D + E189P + N194F + Y242F + | 6.0 | 4.3 |
| E259G + N279S + K280Y + G303R + E390A + G475K | | |
| H1* + G7A + D118T + W140Y + R180* + S181* + T182S + E189P + N194F + Y242F + | 5.7 | 4.1 |
| E259G + N279S + K280Y + G303R + E390A + G475K | | |
| H1* + G7A + W140Y + R180* + S181* + E189P + N194F + Y242F + E259G + | 6.1 | 4.5 |
| N279S + K280Y + G303R + E390A + G475K | | |
| H1* + G7A + W140Y + R180* + S181* + E189P + N194F + Y242F + E259G + | 6.4 | 4.7 |
| N279S + K280Y + G303R + E390A + G475K | | |
| H1* + G7A + W140Y + R180* + S181* + N 194F + L205Y + Y242F + E259G + | 5.4 | 3.9 |
| F266Y + N279S + G303R + E390A + G475K | | |
| H1* + G7A + W140Y + R180* + S181* + N194F + L205Y + Y242F + E259G + | 5.7 | 4.2 |
| N279S + S302T + G303R + E390A + G475K | | |
| H1* + G7A + W140Y + R180* + S181* + N194F + L205Y + Y242F + E259G + | 5.2 | 3.8 |
| N279S + G303R + E390A + N474F + G475K | | |
| H1* + G7A + W140Y + R180* + S181* + N194F + L205Y + Y242F + E259G + | 5.2 | 3.8 |
| N279S + G303R + E390A + N422V + G475K | | |
| H1* + G7A + W140Y + R180* + S181* + N194F + L205Y + Y242F + E259G + | 5.5 | 4.0 |
| N279S + G303R + E390A + G476A | | |
| H1* + G7A + W140Y + R180* + S181* + N194F + L205Y + Y242F + E259G + | 5.5 | 4.0 |
| N279S + G303R + N394W + G475K | | |
| H1* + G7A + W140Y + R180* + S181* + N194F + L205Y + Y242F + E259G + | 5.3 | 3.9 |
| N279S + G303R + E390A + N474L | | |
| H1* + G7A + W140Y + R180* + S181* + N194F + L205Y + Y242F + E259G + | 5.3 | 3.8 |
| N279S + S302T + E390A + G475K | | |
| H1* + G7A + W140Y + R180* + S181* + N194F + L205Y + Y242F + E259G + | 5.5 | 4.0 |
| N279S + G303R + E390A + G475E | | |
| H1* + G7A + W140Y + R180* + S181* + N194F + L205Y + Y242F + E259G + | 5.5 | 4.0 |
| F261P + N279S + G303R + E390A + G475K | | |
| H1* + G7A + W140Y + R180* + S181* + N194F + L205Y + Y242F + E259G + | 4.6 | 3.3 |
| N279S + G303R + E390A + N474F | | |
| H1* + G7A + W140Y + R180* + S181* + N194F + L205Y + Y242F + E259G + | 5.5 | 4.0 |
| N279S + S302Y + E390A + G475K | | |
| H1* + G7A + W140Y + R180* + S181* + N194F + L205Y + Y242F + E259G + | 5.6 | 4.1 |
| N279S + G303R + E390A + N470A + G475K | | |
| H1* + G7A + W140Y + R180* + S181* + N194F + L205Y + Y242F + T256D + | 5.3 | 3.8 |
| N279S + G303R + E390A + G475K | | |
| H1* + G7A + W140Y + R180* + S181* + N194F + L205Y + Y242L + E259G + | 4.6 | 3.4 |
| N279S + G303R + W346N + E390A + G475K | | |
| H1* + G7A + W140Y + R180* + S181* + N194F + L205Y + Y242F + E259G + | 5.5 | 4.0 |
| N279S + G303R + E390A + H420P + G475K | | |
| H1* + G7A + R26Y + W140Y + R180* + S181* + N194F + L205Y + Y242F + | 4.4 | 3.2 |
| E259G + N279S + G303R + E390A + G475K | | |
| H1* + G7A + R37A + W140Y + R180* + S181* + N194F + L205Y + Y242F + | 5.4 | 3.9 |
| E259G + N279S + G303R + G475K | | |
| H1* + W140Y + R180* + S181* + N194F + L205Y + Y242F + E259G + N279S + | 5.2 | 3.8 |
| G303R + Q360Y + E390A + G475K | | |
| H1* + G7A + R37N + W140Y + R180* + S 81* + N194F + L205Y + Y242F + | 5.2 | 3.8 |
| E259G + N279S + G303R + G475K | | |
| H1* + W140Y + R180* + S181* + N194F + L205Y + Y242F + E259G + N279S + | 4.6 | 3.4 |
| G303R + E390A + R414M + G475K | | |
| H1* + W140Y + R180* + S181* + N 194F + L205Y + Y242F + E259G + F261P + | 5.5 | 4.0 |
| N279S + G303R + E390A + G475K | | |
| H1* + W140Y + R180* + S181* + N194F + L205Y + Y242F + E259G + N279S + | 5.3 | 3.8 |
| G303R + H323W + E390A + G475K | | |
| H1* + W140Y + R180* + S181* + N194F + L205Y + Y242F + E259G + N279S + | 3.9 | 2.8 |
| G303R + P321Y + E390A + G475K | | |
| H1* + W140Y + R180* + S181* + N194F + L205Y + Y242F + E259G + N279S + | 6.0 | 4.4 |
| G303R + Q360M + E390A + G475K | | |
| H1* + G7A + W140Y + R180* + S181* + N194F + L205Y + Y242F + E259G + | 5.8 | 4.2 |
| N279S + G303R + Y371S + E390A + G475K | | |
| H1* + G7A + W140Y + R180* + S181* + T182D + A1 85Q + N194F + Y242F + | 6.2 | 4.5 |
| S243Q + E259G + N279S + K280Y + G303R + E390A + G475K | | |
| H1* + G7A + W140Y + R180* + S181* + T182D + Y242F + S243Q + E259G + | 9.3 | 6.8 |
| F266Y + N279S + K280Y + G303R + E390A + G475K | | |
| H1* + G7A + W140Y + R180* + S181* + A185Q + N194F + Y242F + S243Q + | 6.3 | 4.6 |
| E259G + F266Y + N279S + K280Y + G303R + E390A + G475K | | |
| H1* + G7A + W140Y + R180* + S181* + T182D + A185Q + Y242F + E259G + | 6.4 | 4.7 |
| N279S + K280Y + G303R + E390A + G475K | | |
| H1* + G7A + W140Y + R180* + S181* + T182D + E189P + Y242F + S243Q + | 6.5 | 4.7 |
| E259G + N279S + K280Y + G303R + E390A + G475K | | |
| H1* + G7A + W140Y + R180* + S181* + A185Q + Y242F + E259G + F266Y + | 6.0 | 4.4 |
| N279S + K280Y + G303R + E390A + T427L + G475K | | |
| H1* + G7A + W140Y + R180* + S181* + A185Q + E189P + Y242F + E259G + | 6.5 | 4.7 |
| N279S + K280Y + G303R + E390A + G475K | | |
| H1* + G7A + W140Y + R180* + S181* + T182D + A185Q + E189P + N194F + | 6.0 | 4.3 |
| Y242F + S243Q + E259G + N279S + K280Y + G303R + E390A + G475K | | |
| H1* + G7A + W140Y + R180* + S181* + T182D + A185Q + E189P + N194F + | 5.5 | 4.0 |
| L205Y + Y242F + S243Q + E259G + N279S + K280Y + G303R + E390A + G475K | | |
| H1* + G7A + W140Y + R180* + S181* + T182D + E189P + L205Y + Y242F + | 5.6 | 4.1 |
| E259G + F266Y + N279S + K280Y + G303R + E390A + G475K | | |
| H1* + G7A + W140Y + R180* + S181* + A185Q + E189P + N194F + Y242F + | 5.7 | 4.1 |
| E259G + F266Y + N279S + K280Y + G303R + E390A + G475K | | |
| H1* + G7A + W140Y + R180* + S181* + A185Q + E189P + N194F + Y242F + | 5.9 | 4.3 |
| S243Q + E259G + F266Y + N279S + K280Y + G303R + E390A + G475K | | |
| H1* + G7A + W140Y + R180* + S181* + A185Q + E189P + N194F + L205Y + | 5.6 | 4.1 |
| Y242F + S243Q + E259G + F266Y + N279S + K280Y + G303R + E390A + G475K | | |
| H1* + G7A + W140Y + R180* + S181* + T182D + A185Q + E189P + N194F + | 5.7 | 4.1 |
| L205Y + Y242F + E259G + F266Y + N279S + K280Y + G303R + E390A + G475K | | |
| H1* + G7A + W140Y + R180* + S181* + T182D + A185Q + E189P + N194F + | 6.0 | 4.3 |
| Y242F + E259G + F266Y + N279S + K280Y + G303R + E390A + G475K | | |
| H1* + G7A + W140Y + R180* + S181* + A185Q + Y242F + S243Q + E259G + | 6.4 | 4.7 |
| F266Y + N279S + K280Y + G303R + E390A + G475K | | |
| H1* + G7A + W140Y + R180* + S181* + T182D + A185Q + Y242F + S243Q + | 5.9 | 4.3 |
| E259G + F266Y + N279S + G303R + E390A + G475K | | |
| H1* + G7A + W140Y + R180* + S181* + Y242F + S243Q + E259G + N279S + | 5.4 | 4.0 |
| K280Y + G303R + E390A + G475K | | |
| H1* + G7A + W140Y + R180* + S181* + T182D + E189P + N194F + Y242F + | 5.6 | 4.0 |
| E259G + N279S + K280Y + G303R + E390A + G475K | | |
| H1* + G7A + W140Y + R1 80* + S181* + A185Q + N194F + Y242F + S243Q + | 6.6 | 4.8 |
| E259G + N279S + K280Y + G303R + E390A + G475K | | |
| H1* + G7A + W140Y + R180* + S181* + T182D + N194F + Y242F + S243Q + | 5.2 | 3.8 |
| E259G + N279S + K280Y + G303R + E390A + G475K | | |
| H1* + G7A + W140Y + R180* + S181* + A185Q + E189P + N194F + Y242F + | 5.6 | 4.1 |
| E259G + N279S + K280Y + G303R + E390A + G475K | | |
| H1* + G7A + W140Y + R180* + S181* + E189P + Y242F + S243Q + E259G + | 5.6 | 4.1 |
| N279S + K280Y + G303R + E390A + G475K | | |
| H1* + G7A + W140Y + R180* + S181* + T182D + Y242F + S243Q + E259G + | 5.1 | 3.7 |
| N279S + K280Y + G303R + E390A + G475K | | |
| H1* + G7A + W140Y + R180* + S181* + E189P + L205Y + Y242F + S243Q + | 4.5 | 3.2 |
| E259G + N279S + K280Y + G303R + E390A + G475K | | |
| H1* + G7A + W140Y + R180* + S181* + Y242F + E259G + F266Y + N279S + | 5.2 | 3.8 |
| K280Y + G303R + E390A + G475K | | |
| H1* + G7A + W140Y + R180* + S181* + T182D + A185Q + Y242F + E259G + | 5.0 | 3.7 |
| N279S + G303R + E390A + G475K | | |
| H1* + G7A + W140Y + R180* + S181* + T182D + A185Q + N194F + Y242F + | 5.1 | 3.7 |
| S243Q + E259G + N279S + G303R + E390A + G475K | | |
| H1* + G7A + W140Y + R180* + S181* + T182D + N194F + Y242F + S243Q + | 4.7 | 3.4 |
| E259G + F266Y + N279S + G303R + E390A + G475K | | |
| H1* + G7A + W140Y + R180* + S181* + T182D + E189P + N194F + Y242F + | 4.1 | 3.0 |
| S243Q + E259G + N279S + K280Y + G303R + E390A + G475K | | |
| H1* + G7A + W140Y + R180* + S181* + E189P + N194F + Y242F + E259G + | 5.9 | 4.3 |
| YF266 + N279S + K280Y + G303R + E390A + G475K | | |
| H1* + G7A + W140Y + R180* + S181* + A185Q + E189P + N194F + L205Y + | 5.0 | 3.6 |
| Y242F + S243Q + E259G + N279S + K280Y + G303R + E390A + G475K | | |
| H1* + G7A + W140Y + R180* + S181* + T182D + A185Q + N194F + Y242F + | 5.2 | 3.8 |
| E259G + F266Y + N279S + G303R + E390A + G475K | | |
| H1* + G7A + W140Y + R180* + S181* + A185Q + N194F + Y242F + E259G + | 5.1 | 3.7 |
| F266Y + N279S + K280Y + G303R + E390A + G475K | | |
| H1* + G7A + W140Y + R180* + S181* + A185Q + E189P + L205Y + Y242F + | 4.1 | 3.0 |
| E259G + N279S + K280Y + G303R + E390A + G475K | | |
| H1* + G7A + W140Y + R180* + S181* + Y242F + S243Q + E259G + F266Y + | 5.0 | 3.7 |
| *N279S + K280Y + G303R + E390A + G475K | | |
| H1* + G7A + W140Y + R180* + S181* + T182D + E189P + L205Y + Y242F + | 3.9 | 2.8 |
| S243Q + E259G + N279S + K280Y + G303R + E390A + I409L + G475K | | |
| H1* + G7A + A41D + W140Y + R180* + S181* + N194F + L205Y + Y242F + | 7.4 | 5.4 |
| E259G + N279S + G303R + E390A + G475K | | |
| H1* + G7A + T40G + W140Y + R180* + S181* + N 194F + L205Y + Y242F + | 7.9 | 5.7 |
| E259G + N279S + G303R + E390A + G475K | | |
| L63F + W140Y + L173F + R180* + S181* + N194F + L205Y + Y242F + E259G + | 6.3 | 4.6 |
| N279S + H323W | | |
| W140Y + L173F + R180* + S181* + N194F + L205Y + N225S + Y242F + E259G + | 7.0 | 5.1 |
| N279S + G303R + H323A | | |
| H1* + N3D + G7A + D118T + W140Y + R180* + S181* + T182S + E189P + N194F + | 7.5 | 5.5 |
| L205Y + Y242F + E259G + N279S + K280Y + S302Y + E390A + N474L | | |
| H1* + N3D + G7A + D118T + W140Y + R180* + S181* + T182S + E189P + N194F + | 6.8 | 4.9 |
| L205Y + Y242F + E259G + N279S + K280Y + G303R + Y362 V + E390A + H420A + | | |
| N474L | | |
| H1* + N3D + G7A + D118T + W140Y + R180* + S181* + T182S + E189P + N194F + | 7.8 | 5.7 |
| L205Y + Y242F + E259G + N279S + K280Y + S302T + V365I + E390A + N474L | | |
| H1* + G7A + D118T + W140Y + R180* + S181* + T182S + E189P + N194F + | 7.6 | 5.6 |
| L205Y + Y242F + E259G + N279S + K280Y + S302Y + E390A + H420A + G475K | | |
| H1* + G7A + D118T + W140Y + R180* + S181* + T182S + E189P + N194F + | 6.5 | 4.7 |
| L205Y + Y242F + E259G + N279S + K280Y + S302Y + E390A + H420A + G475K | | |
| H1* + N3D + G7A + D118T + W140Y + R180* + S181* + T182S + E189P + N194F + | 8.5 | 6.2 |
| L205Y + Y242F + E259G + N279S + K280Y + S302Y + E390A + N474L | | |
| H1* + N3D + G7A + D118T + W140Y + R180* + S181* + T182S + E189P + N194F + | 7.5 | 5.5 |
| L205Y + Y242F + E259G + N279S + K280Y + S302T + E390A + H420A + G475K | | |
| H1* + G7A + D118T + W140Y + R180* + S181* + T182S + E189P + N194F + | 7.0 | 5.1 |
| L205Y + Y242F + E259G + N279S + K280Y + S302Y + E390A + H420A + G475K | | |
| H1* + N3D + G7A + D118T + W140Y + R180* + S181* + T182S + E189P + N194F + | 7.2 | 5.3 |
| L205Y + Y242F + E259G + N279S + K280Y + S302T + E390A + H420A + G475K | | |
| H1* + G7A + D118T + W140Y + R180* + S181* + T182S + E189P + N194F + | 8.1 | 5.9 |
| L205Y + Y242F + E259G + N279S + K280Y + G303R + E390A + H420A + G476A | | |
| H1* + N3D + G7A + D118T + W140Y + R180* + S181* + T182S + E189P + N194F + | 7.4 | 5.4 |
| L205Y + Y242F + E259G + N279S + K280Y + S302T + E390A + G475K | | |
| H1* + N3G + G7A + D118T + W140Y + R180* + S181* + T182S + E189P + N194F + | 7.4 | 5.4 |
| L205Y + Y242F + E259G + N279S + K280Y + S302T + E390A + H420A + G475K | | |
| H1* + G7A + D118T + W140Y + R180* + S181* + T182S + E189P + N194F + | 6.8 | 5.0 |
| L205Y + Y242F + E259G + N279S + K280Y + G303R + E390A + H420A + N474L | | |
| H1* + N3D + G7A + R37A + D118T + W140Y + R180* + S181* + T182S + E189P + | 4.1 | 3.0 |
| N194F + L205Y + Y242F + E259G + N279S + K280Y + G303R + N394D + N474F | | |
| H1* + G7A + D118T + W140Y + R180* + S181* + T182S + E189P + N194F + | 6.2 | 4.5 |
| L205Y + Y242F + E259G + N279S + K280Y + G303R + N394W + H420P + N474L | | |
| H1* + G7A + G73L + D118T + W140Y + R180* + S181* + T182S + E189P + | 7.0 | 5.1 |
| N194F + L205Y + Y242F + E259G + N279S + K280Y + S302Y + E390A + G475K | | |
| H1* + G7A + D118T + W140Y + R180* + S181* + T182S + E189P + N194F + | 8.8 | 6.4 |
| L205Y + Y242F + E259G + N279S + K280Y + S302T + E390A + G475K | | |
| H1* + G7A + D118T + W140Y + R180* + S181* + T182S + E189P + N194F + | 7.7 | 5.6 |
| L205Y + Y242F + E259G + N279S + K280Y + S302Y + E390A + N474L | | |
| H1* + G7A + D118T + W140Y + R180* + S181* + T182S + E189P + N194F + | 5.3 | 3.8 |
| L205Y + Y242F + E259G + N279S + K280Y + S302T + E390A + N474L | | |
| H1* + G7A + D118T + W140Y + R180* + S181* + T182S + E189P + N194F + | 4.3 | 3.1 |
| L205Y + Y242F + E259G + N279S + K280Y + G303R + E390A + G476A | | |
| H1* + N3D + G7A + D118T + W140Y + R180* + S181* + T182S + E189P + N194F + | 4.3 | 3.1 |
| L205Y + Y242F + E259G + N279S + K280Y + S302Y + E390A + H420A + G475K | | |
| H1* + N3D + G7A + D118T + W140Y + R180* + S181* + T182S + E189P + N194F + | 4.3 | 3.1 |
| L205Y + Y242F + E259G + N279S + K280Y + S302Y + E390A + G475K | | |
| H1* + G7A + D118T + W140Y + R180* + S181* + T182S + E189P + N194F + | 4.2 | 3.1 |
| L205Y + Y242F + E259G + N279S + K280Y + S302Y + N394W + G475K | | |
| H1* + N3D + G7A + K93R + D118T + W140Y + R180* + S181* + T182S + E189P + | 3.6 | 2.6 |
| N194F + L205Y + Y242F + E259G + N279S + K280Y + G303R + E390A + H420A + | | |
| N474L | | |
| H1* + G7A + R37N + D118T + W140Y + R180* + S181* + T182S + E189P + N194F + | 4.7 | 3.4 |
| L205Y + Y242F + E259G + N279S + K280Y + S302Y + E390A + G475K | | |
| H1* + N3D + G7A + D118T + W140Y + R180* + S181* + T182S + E189P + N194F + | 4.2 | 3.0 |
| L205Y + Y242F + E259G + N279S + K280Y + G303R + E390D + H420P + G475K | | |
| H1* + G7A + D118T + W140Y + R180* + S181* + T182S + E189P + N194F + | 4.6 | 3.4 |
| L205Y + Y242F + E259G + N279S + K280Y + S302T + N394W + G475K | | |
| H1* + N3G + G7A + D118T + W140Y + R180* + S181* + T182S + E189P + N194F + | 4.3 | 3.2 |
| L205Y + Y242F + E259G + N279S + K280Y + S302T + E390A + H420A + G475K | | |
| H1* + N3D + G7A + D118T + W140Y + R180* + S181* + T182S + E189P + N194F + | 4.3 | 3.1 |
| L205Y + Y242F + E259G + N279S + K280Y + G303R + N394W + H420A + G475K | | |
| H1* + G7A + R37N + D118T + W140Y + R180* + S181* + T182S + E189P + N194F + | 4.4 | 3.2 |
| L205Y + Y242F + E259G + N279S + K280Y + G303R + E390A + H420A + G475K | | |
| H1* + N3D + G7A + D118T + W140Y + R180* + S181* + T182S + E189P + N194F + | 4.0 | 2.9 |
| L205Y + Y242F + E259G + N279S + K280Y + S302T + E390A + G476H | | |
| H1* + G7A + D118T + W140Y + R180* + S181* + T182S + E189P + N194F + | 4.1 | 3.0 |
| L205Y + Y242F + E259G + N279S + K280Y + G303R + N394D + H420A + G475K | | |
| H1* + N3D + G7A + D118T + W140Y + R180* + S181* + T182S + E189P + N194F + | 4.1 | 3.0 |
| L205Y + Y242F + E259G + N279S + K280Y + G303R + E390A + H420A + G475K | | |
| H1* + G7A + D118T + W140Y + R180* + S181* + T182S + E189P + N194F + | 4.2 | 3.0 |
| L205Y + Y242F + E259G + N279S + K280Y + S302T + E390A + G476A | | |
| H1* + G7A + D118T + W140Y + R180* + S181* + T182S + E189P + N194F + | 4.0 | 2.9 |
| L205Y + Y242F + E259G + N279S + K280Y + G303R + E390A + H420P + G475K | | |
| H1* + N3D + G7A + D118T + W140Y + R180* + S181* + T182S + E189P + N194F + | 3.7 | 2.7 |
| L205Y + Y242F + E259G + N279S + K280Y + G303R + E390A + H420A + N474L | | |
| N3G + G7A + D118T + W140Y + R180* + S181* + T182S + E189P + N194F + | 3.7 | 2.7 |
| L205Y + Y242F + E259G + N279S + K280Y + G303R + E390A + H420A + N474L | | |
| H1* + G7A + R37N + D118T + W140Y + R180* + S181* + T182S + E189P + N194F + | 4.3 | 3.1 |
| L205Y + Y242F + E259G + N279S + K280Y + G303R + N394W + H420A + G475K | | |
| H1* + N3D + G7A + R37N + D118T + Q138V + W140Y + R180* + S181* + T182S + | 4.4 | 3.2 |
| E189P + N194F + L205Y + Y242F + E259G + N279S + K280Y + G303R + E390A + | | |
| G475K | | |
| H1* + G7A + D118T + W140Y + R180* + S181* + T182S + E189P + N194F + | 4.2 | 3.1 |
| L205Y + Y242F + E259G + N279S + K280Y + S302Y + E390A + G476H | | |
| H1* + G7A + D118T + Y135 E + W140Y + R180* + S181* + T182S + E189P + | 4.5 | 3.3 |
| N194F + L205Y + Y242F + E259G + N279S + K280Y + S302T + E390A + N474L | | |
| H1* + G7A + D118T + W140Y + R180* + S181* + T182S + E189P + N194F + | 4.6 | 3.4 |
| L205Y + Y242F + E259G + N279S + K280Y + G303R + N394W + H420A + G475K | | |
| H1* + G7A + D118T + W140Y + R180* + S181* + T182S + E189P + N194F + | 4.2 | 3.1 |
| L205Y + Y242F + E259G + N279S + K280Y + S302Y + E390A + G475K | | |
| H1* + N3D + G7A + D118T + W140Y + R180* + S181* + T182S + E189P + N194F + | 3.7 | 2.7 |
| L205Y + Y242F + E259G + N279S + K280Y + G303R + E390A + H420P + G475K | | |
| H1* + G7A + V103I + D118T + W140Y + R180* + S181* + T182S + E189P + N194F + | 3.9 | 2.8 |
| L205Y + Y242F + E259G + N279S + K280Y + S302Y + E390A + N474L | | |
| H1* + G7A + D118T + W140Y + R180* + S181* + T182S + E189P + N194F + | 4.4 | 3.2 |
| L205Y + Y242F + E259G + M260D + N279S + K280Y + S302Y + E390A + G475K | | |
| H1* + G7A + D118T + W140Y + R180* + S181* + T182S + E189P + N194F + | 4.1 | 2.9 |
| L205Y + Y242F + E259G + N279S + K280Y + S302Y + E390A + N474L | | |
| H1* + G7A + D118T + W140Y + R180* + S181* + T182S + E189P + N194F + | 4.4 | 3.2 |
| L205Y + Y242F + E259G + N279S + K280Y + S302T + E390A + G475K | | |
| H1* + G7A + D118T + Q138 V + W140Y + R180* + S181* + T182S + E189P + | 4.0 | 2.9 |
| N194F + L205Y + Y242F + E259G + N279S + K280Y + G303R + H323W + E390A + | | |
| N474L | | |
| H1* + G7A + D118T + W140Y + R180* + S181* + T182S + E189P + N194F + | 4.4 | 3.2 |
| L205Y + Y242F + E259G + N279S + K280Y + S302Y + E390A + G476H | | |
| H1* + G7A + D118T + W140Y + R180* + S181* + T182S + E189P + N194F + | 4.5 | 3.3 |
| L205Y + Y242F + E259G + N279S + K280Y + S302T + E390A + N474L | | |
| H1* + G7A + D118T + W140Y + P146H + R180* + S181* + T182S + E189P + | 4.3 | 3.1 |
| N194F + L205Y + Y242F + E259G + N279S + K280Y + S302T + E390A + G475K | | |
| H1* + G7A + D118T + W140Y + R180* + S181* + T182S + E189P + N194F + | 4.1 | 3.0 |
| L205Y + Y242F + E259G + F261P + N279S + K280Y + G303R + E390A + G475K | | |
| H1* + G7A + T40G + D118T + W140Y + R180* + S181* + T182S + E189P + | 4.3 | 3.1 |
| N194F + L205Y + Y242F + E259G + N279S + K280Y + S302Y + E390A + G475K | | |
| H1* + G7A + Q98L + D118T + W140Y + R180* + S181* + T182S + E189P + | 4.0 | 2.9 |
| N194F + L205Y + Y242F + E259G + N279S + K280Y + S302Y + E390A + G475K | | |
| H1* + G7A + D118T + W140Y + R180* + S181* + T182S + E189P + N194F + | 3.6 | 2.6 |
| L205Y + Y242F + E259G + N279S + K280Y + G303R + E390A + N474L | | |
| H1* + G7A + D118T + W140Y + R180* + S181* + T182S + E189P + N194F + | 3.7 | 2.7 |
| L205Y + Y242F + E259G + N279S + K280Y + G303R + P321H + E390A + G475K | | |
| H1* + G7A + D118T + W140Y + R180* + S181* + T182S + E189P + N194F + | 3.7 | 2.7 |
| L205Y + Y242F + E259G + N279S + K280Y + G303R + P321H + E390A + N474L | | |
| H1* + G7A + D118T + S132D + W140Y + R180* + S181* + T182S + E189P + | 3.9 | 2.8 |
| N194F + L205Y + Y242F + E259G + N279S + K280Y + S302Y + E390A + G475K | | |
| H1* + G7A + D118T + W140Y + R180* + S181* + T182S + E189P + N194F + | 3.7 | 2.7 |
| L205Y + Y242F + E259G + N279S + K280Y + G303R + N394D + R414M + G475K | | |
| H1* + G7A + T40G + D118T + W140Y + R180* + S181* + T182S + E189P + | 4.4 | 3.2 |
| N194F + L205Y + Y242F + E259G + N279S + K280Y + G303R + E390A + G476A | | |
| H1* + G7A + D118T + W140Y + R180* + S181* + T182S + E189P + N194F + | 4.2 | 3.0 |
| L205Y + T224F + Y242F + E259G + N279S + K280Y + G303R + N394W + H420A + | | |
| G475K | | |
| H1* + G7A + D118T + W140Y + R180* + S181* + T182S + E189P + N194F + | 4.8 | 3.5 |
| L205Y + Y242F + E259G + M260D + N279S + K280Y + S302T + E390A + G475K | | |
| H1* + G7A + D118T + W140Y + R180* + S181* + T182S + E189P + N194F + | 4.6 | 3.4 |
| L205Y + Y242F + E259G + M260W + N279S + K280Y + S302Y + E390A + G476A | | |
| H1* + G7A + D118T + Y135E + W140Y + R180* + S181* + T182S + E189P + | 4.3 | 3.1 |
| N194F + L205Y + Y242F + E259G + M260D + N279S + K280Y + G303R + W346N + | | |
| E390A + G475K | | |
| H1* + G7A + D118T + W140Y + R180* + S181* + T182S + E189P + N194F + L205Y + | 4.6 | 3.3 |
| Y242F + E259G + N279S + K280Y + G303R + P321H + Q360Y + E390A + G476A | | |
| H1* + G7A + D118T + W140Y + R180* + S181* + T182S + E189P + N194F + L205Y + | 5.3 | 3.9 |
| Y242F + E259G + M260D + N279S + K280Y + S302Y + E390A + N470M + G475K | | |
| H1* + G7A + D118T + W140Y + R180* + S181* + T182S + E189P + N194F + | 6.0 | 4.4 |
| L205Y + Y242F + E259G + M260W + N279S + K280Y + G303R + E390A + G476A | | |
| H1* + G7A + D118T + W140Y + R180* + S181* + T182S + E189P + N194F + | 6.1 | 4.5 |
| L205Y + T224Y + Y242F + N279S + K280Y + G303R + E390A + G476A | | |
| H1* + G7A + D118T + W140Y + R180* + S181* + T182S + E189P + N194F + | 6.4 | 4.6 |
| L205Y + Y242F + E259G + N279S + K280Y + S302T + E390A + G475K | | |
| H1* + G7A + D118T + W140Y + R180* + S181* + T182S + E189P + N194F + | 5.5 | 4.0 |
| L205Y + Y242F + E259G + N279S + K280Y + S302Y + E390A + N474L | | |
| H1* + G7A + D118T + W140Y + R180* + S181* + T182S + E189P + N194F + | 5.8 | 4.2 |
| L205Y + Y242F + E259G + N279S + K280Y + S302Y + E390A + G476H | | |
| H1* + G7A + D118T + W140Y + R180* + S181* + T182S + E189P + N194F + | 4.8 | 3.5 |
| L205Y + Y242F + E259G + N279S + K280Y + S302Y + E390A + N474F | | |
| H1* + G7A + D118T + W140Y + R180* + S181* + T182S + E189P + N194F + | 5.8 | 4.2 |
| L205Y + Y242F + E259G + M260W + N279S + K280Y + G303R + E390A + G476A | | |
| H1* + G7A + D118T + W140Y + R180* + S181* + T182S + E189P + N194F + | 5.6 | 4.1 |
| L205Y + Y242F + E259G + M260W + N279S + K280Y + S302Y + E390A + G475K | | |
| H1* + N3D + G7A + D118T + W140Y + R180* + S181* + T182S + E189P + N194F + | 6.1 | 4.4 |
| L205Y + Y242F + E259G + N279S + K280Y + S302T + E390A + H420A + G475K | | |
| H1* + G7A + D118T + W140Y + R180* + S181* + T182S + E189P + N194F + | 4.7 | 3.4 |
| L205Y + T224Y + Y242F + E259G + N279S + K280Y + G303R + E390A + N474L | | |
| H1* + G7A + D118T + W140Y + R180* + S181* + T182S + E189P + N194F + | 5.5 | 4.0 |
| L205Y + Y242F + E259G + N279S + K280Y + S302Y + E390A + G475K | | |
| H1* + G7A + D118T + W140Y + R180* + S181* + T182S + E189P + N194F + | 6.0 | 4.3 |
| L205Y + Y242F + E259G + N279S + K280Y + S302Y + E390A + H420A + G475K | | |
| H1* + G7A + D118T + W140Y + R180* + S181* + T182S + E189P + N194F + | 6.4 | 4.7 |
| L205Y + Y242F + E259G + N279S + K280Y + G303R + E390A + G476A | | |
| H1* + G7A + D118T + W140Y + R180* + S181* + T182S + E189P + N194F + | 5.5 | 4.0 |
| L205Y + Y242F + E259G + N279S + K280Y + S302T + E390A + N474L | | |
| H1* + G7A + D118T + W140Y + P146H + R180* + S181* + T182S + E189P + | 6.5 | 4.7 |
| N194F + L205Y + Y242F + E259G + N279S + K280Y + S302T + E390A + G475K | | |
| H1* + G7A + D118T + W140Y + R180* + S181* + T182S + E189P + N194F + | 6.8 | 4.9 |
| L205Y + Y242F + E259G + N279S + K280Y + S302Y + E390A + G475K | | |
| H1* + N3G + G7A + D118T + W140Y + R180* + S181* + T182S + E189P + N194F + | 6.0 | 4.3 |
| L205Y + Y242F + E259G + N279S + K280Y + S302T + E390A + G475K | | |
| H1* + G7A + D118T + S132L + W140Y + R180* + S181* + T182S + E189P + N194F + | 6.2 | 4.5 |
| L205Y + Y242F + E259G + M260D + N279S + K280Y + G303R + E390A + G475K | | |
| H1* + G7A + D118T + W140Y + R180* + S181* + T182S + E189P + N194F + | 6.4 | 4.7 |
| L205Y + Y242F + E259G + N279S + K280Y + S302T + E390A + G475K | | |
| H1* + G7A + D118T + W140Y + R180* + S181* + T182S + E189P + N194F + | 5.1 | 3.7 |
| L205Y + Y242F + E259G + N279S + K280Y + G303R + E390A + G475K | | |
| H1* + D118T + W140Y + R180* + S181* + T182S + E189P + N194F + L205Y + | 2.9 | 2.1 |
| Y242F + E259G + N279S + K280Y + G303R + E390A + G475K | | |
| H1* + G7A + W140Y + R180* + S181* + T182S + E189P + N194F + L205Y + | 5.2 | 3.8 |
| Y242F + E259G + N279S + K280Y + G303R + E390A + G475K | | |
| H1* + G7A + D118T + R180* + S181* + T182S + E189P + N194F + L205Y + | 4.1 | 3.0 |
| Y242F + E259G + N279S + K280Y + G303R + E390A + G475K | | |
| H1* + G7A + D118T + W140Y + R180* + S181* + E189P + N194F + L205Y + | 5.2 | 3.8 |
| Y242F + E259G + N279S + K280Y + G303R + E390A + G475K | | |
| H1* + G7A + D118T + W140Y + R180* + S181* + T182S + N194F + L205Y + | 5.9 | 4.3 |
| Y242F + E259G + N279S + K280Y + G303R + E390A + G475K | | |
| H1* + G7A + D118T + W140Y + R180* + S181* + T182S + E189P + L205Y + | 4.5 | 3.3 |
| Y242F + E259G + N279S + K280Y + G303R + E390A + G475K | | |
| H1* + G7A + D118T + W140Y + R180* + S181* + T182S + E189P + N194F + | 5.4 | 3.9 |
| Y242F + E259G + N279S + K280Y + G303R + E390A + G475K | | |
| H1* + G7A + D118T + W140Y + R180* + S181* + T182S + E189P + N194F + | 5.2 | 3.8 |
| L205Y + E259G + N279S + K280Y + G303R + E390A + G475K | | |
| H1* + G7A + D118T + W140Y + R180* + S181* + T182S + E189P + N194F + | 5.6 | 4.0 |
| L205Y + Y242F + N279S + K280Y + G303R + E390A + G475K | | |
| H1* + G7A + D118T + W140Y + R180* + S181* + T182S + E189P + N194F + | 4.6 | 3.4 |
| L205Y + Y242F + E259G + K280Y + G303R + E390A + G475K | | |
| H1* + G7A + D118T + W140Y + R180* + S181* + T182S + E189P + N194F + | 3.3 | 2.4 |
| L205Y + Y242F + E259G + N279S + G303R + E390A + G475K | | |
| H1* + G7A + D118T + W140Y + R180* + S181* + T182S + E189P + N194F + | 5.6 | 4.1 |
| L205Y + Y242F + E259G + N279S + K280Y + E390A + G475K | | |
| H1* + G7A + D118T + W140Y + R180* + S181* + T182S + E189P + N194F + | 5.1 | 3.7 |
| L205Y + Y242F + E259G + N279S + K280Y + G303R + G475K | | |
| H1* + G7A + D118T + W140Y + R180* + S181* + T182S + E189P + N194F + | 4.7 | 3.4 |
| L205Y + Y242F + E259G + N279S + K280Y + G303R + E390A | | |
| H1* + G7A + D118T + W140Y + R180* + S181* + T182S + E189P + N194F + | 5.6 | 4.0 |
| L205V + Y242F + E259G + N279S + K280Y + G303R + E390A + G475K | | |
| H1* + G7A + D118T + W140Y + R180* + S181* + T182S + E189P + N194F + | 5.5 | 4.0 |
| L205N + Y242F + E259G + N279S + K280Y + G303R + E390A + G475K | | |
| H1* + G7A + D118T + W140Y + R180* + S181* + T182S + E189P + N194F + | 4.5 | 3.3 |
| L205Q + Y242F + E259G + N279S + K280Y + G303R + E390A + G475K | | |
| H1* + G7A + D118T + W140Y + R180* + S181* + T182S + A185Q + E189P + | 5.0 | 3.6 |
| N194F + L205Y + Y242F + E259G + N279S + K280Y + G303R + E390A + G475K | | |
| H1* + G7A + D118T + W140Y + R180* + S181* + T182S + E189P + N194F + | 5.0 | 3.6 |
| L205Y + Y242F + E259G + F266Y + N279S + K280Y + G303R + E390A + G475K | | |
| H1* + G7A + D118T + W140Y + R180* + S181* + A185Q + E189P + N194F + | 5.1 | 3.7 |
| L205Y + Y242F + E259G + N279S + K280Y + G303R + E390A + G475K | | |
| H1* + G7A + D118T + W140Y + R180* + S181* + T182S + A185Q + E189P + | 5.2 | 3.7 |
| N194F + L205Y + Y242F + E259G + F266Y + N279S + K280Y + G303R + E390A + | | |
| G475K | | |
| H1* + G7A + D118T + W140Y + R180* + S181* + T182S + A185Q + E189P + | 4.3 | 3.1 |
| N194F + L205N + Y242F + E259G + F266Y + N279S + K280Y + G303R + E390A + | | |
| G475K | | |
| H1* + G7A + D118T + W140Y + R180* + S181* + T182S + A185D + E189P + | 5.8 | 4.2 |
| N194F + Y242F + E259G + F266Y + N279S + K280Y + G303R + E390A + G475K | | |
| H1* + G7A + D118T + W140Y + R180* + S181* + T182S + A185D + E189P + | 5.3 | 3.8 |
| Y242F + S243Q + E259G + F266Y + N279S + K280Y + G303R + E390A + G475K | | |
| H1* + G7A + D118T + W140Y + R180Q + S181* + T182* + A185Q + E189P + | 5.2 | 3.8 |
| Y242F + S243Q + E259G + F266Y + N279S + K280Y + G303R + E390A + G475K | | |
| H1* + G7A + D118T + W140Y + R180* + S181* + T182S + A185Q + E189P + | 5.2 | 3.8 |
| Y242F + E259G + F266Y + N279S + K280Y + G303R + E390A + G475K | | |
| H1* + G7A + D118T + W140Y + R180* + S181* + T182S + A185Q + E189P + | 4.4 | 3.2 |
| L205Y + Y242F + E259G + F266Y + N279S + K280Y + G303R + E390A + G475K | | |
| H1* + G7A + D118T + W140Y + R180* + S181* + T182S + A185D + E189P + | 4.9 | 3.6 |
| L205Y + Y242F + E259G + F266Y + N279S + K280Y + G303R + E390A + G475K | | |
| H1* + G7A + D118T + W140Y + R180Q + S181* + T182* + A 185Q + E189P + | 5.0 | 3.6 |
| N194F + L205Y + Y242F + E259G + F266Y + N279S + K280Y + G303R + E390A + | | |
| G475K | | |
| H1* + N3D + G7A + D118T + W140Y + R180* + S181* + T182S + E189P + N194F + | 6.0 | 4.4 |
| L205Y + Y242F + E259G + N279S + K280Y + S302T + E390A + H420A + G475K | | |
| H1* + G7A + T40G + D118T + W140Y + R180* + S181* + T182S + E189P + N194F + | 4.6 | 3.3 |
| L205Y + Y242F + E259G + N279S + K280Y + S302Y + E390A + G475K | | |
| H1* + G7A + T40G + D118T + W140Y + R180* + S181* + T182S + E189P + N194F + | 4.6 | 3.3 |
| L205Y + Y242F + E259G + N279S + K280Y + S302Y + E390A + G475K | | |
| H1* + G7A + T40G + D118T + W140Y + R180* + S181* + T182S + E189P + N194F + | 5.0 | 3.6 |
| L205Y + Y242F + E259G + N279S + K280Y + G303R + E390A + G476A | | |
| H1* + G7A + T40G + D118T + W140Y + R180* + S181* + T182S + E189P + N194F + | 5.2 | 3.7 |
| L205Y + Y242F + E259G + N279S + K280Y + G303R + E390A + G476A | | |
| H1* + G7A + D118T + W140Y + R180* + S181* + T182S + E189P + N194F + | 4.5 | 3.2 |
| L205Y + Y242F + E259G + N279S + K280Y + G303R + N394D + N422V + G475K | | |
| H1* + G7A + Q98L + D118T + W140Y + R180* + S181* + T182S + E189P + N194F + | 4.7 | 3.4 |
| L205Y + Y242F + E259G + N279S + K280Y + G303R + N394D + H420A + G475K | | |
| H1* + G7A + D118T + W140Y + R180* + S181* + T182S + E189P + N194F + L205Y + | 5.5 | 4.0 |
| Y242F + E259G + M260D + N279S + K280Y + G303R + Y362V + E390A + G475K | | |
| H1* + G7A + D118T + Y135E + W140Y + R180* + S181* + T182S + E189P + | 4.8 | 3.5 |
| N194F + L205Y + Y242F + E259G + F261P + N279S + K280Y + G303R + Y362V + | | |
| E390A + G475K | | |
| H1* + G7A + D118T + W140Y + R180* + S181* + T182S + E189P + N194F + | 5.4 | 3.9 |
| L205Y + Y242F + E259G + M260D + N279S + K280Y + G303R + E390A + G475K | | |
| H1* + G7A + D118T + W140Y + R180* + S181* + T182S + E189P + N194F + | 4.5 | 3.3 |
| L205Y + Y242F + T256D + N279S + K280Y + G303R + E390A + G475K | | |
| H1* + G7A + D118T + Y135 E + W140Y + R180* + S181* + T182S + E189P + | 5.2 | 3.7 |
| N194F + L205Y + T224F + Y242F + E259G + N279S + K280Y + G303R + E390A + | | |
| G475K | | |
| H1* + G7A + D118T + W140Y + R180* + S181* + T182S + E189P + N194F + | 4.5 | 3.3 |
| L205Y + Y242F + T256D + N279S + K280Y + G303R + E390A + G475K | | |
| H1* + G7A + D118T + W140Y + R180* + S181* + T182S + E189P + N194F + | 4.2 | 3.1 |
| L205Y + Y242F + T256D + N279S + K280Y + G303R + E390A + G475K | | |
| H1* + G7A + D118T + W140Y + R180* + S181* + T182S + E189P + N194F + | 4.8 | 3.5 |
| L205Y + Y242F + E259G + F261P + N279S + K280Y + G303R + E390A + G475K | | |
| H1* + G7A + D118T + W140Y + R180* + S181* + T182S + E189P + N194F + | 4.4 | 3.2 |
| L205Y + Y242F + T256D + N279S + K280Y + G303R + E390A + G475K | | |
| H1* + G7A + D118T + W140Y + R180* + S181* + T182S + E189P + N194F + | 4.3 | 3.1 |
| L205Y + Y242F + E259G + M260D + N279S + K280Y + G303R + Y371N + E390A + | | |
| G475K | | |
| H1* + G7A + D118T + W140Y + R180* + S181* + T182S + E189P + N194F + | 5.8 | 4.2 |
| L205Y + Y242F + E259G + N279S + K280Y + S302T + E390A + G475K | | |
| H1* + G7A + D118T + W140Y + R180* + S181* + T182S + E189P + N194F + | 4.8 | 3.5 |
| L205Y + Y242F + E259G + N279S + K280Y + S302T + E390A + N474F | | |
| H1* + G7A + D118T + Y135 E + W140Y + R180* + S181* + T182S + E189P + | 4.8 | 3.5 |
| N194F + L205Y + Y242F + E259G + N279S + K280Y + G303R + P321Y + E390A + | | |
| G475K | | |
| H1* + G7A + D118T + S132D + W140Y + R180* + S181* + T182S + E189P + | 5.3 | 3.8 |
| N194F + L205Y + Y242F + E259G + N279S + K280Y + G303R + P321Y + E390A + | | |
| G475K | | |
| H1* + N3G + G7A + D118T + Q138V + W140Y + R180* + S181* + T182S + E189P + | 5.3 | 3.9 |
| N194F + L205Y + Y242F + E259G + N279S + K280Y + G303R + E390A + H420A + | | |
| G476A | | |
| H1* + G7A + D118T + W140Y + R180* + S181* + T182S + E189P + N194F + | 5.9 | 4.3 |
| L205Y + Y242F + E259G + N279S + K280Y + S302Y + E390A + G475K | | |
| H1* + N3D + G7A + D118T + W140Y + R180* + S181* + T182S + E189P + N194F + | 6.0 | 4.4 |
| L205Y + Y242F + E259G + N279S + K280Y + S302T + E390A + G475K | | |
| H1* + G7A + D118T + Q138 V + W140Y + R180* + S181* + T182S + E189P + | 5.9 | 4.3 |
| N194F + L205Y + Y242F + E259G + N279S + K280Y + S302T + E390A + G475K | | |
| H1* + G7A + D118T + W140Y + R180* + S181* + T182S + E189P + N194F + | 5.7 | 4.2 |
| L205Y + Y242F + E259G + N279S + K280Y + S302T + E390A + G475K | | |
| H1* + G7A + D118T + W140Y + R180* + S181* + T182S + E189P + N194F + | 5.8 | 4.2 |
| L205Y + Y242F + E259G + N279S + K280Y + S302T + E390A + G475K | | |
| H1* + G7A + D118T + W140Y + R180* + S181* + T182S + E189P + N194F + | 4.6 | 3.3 |
| L205Y + Y242F + T256D + N279S + K280Y + G303R + E390A + N470M + G475K | | |
| H1* + N3D + G7A + D118T + W140Y + R180* + S181* + T182S + E189P + N194F + | 5.3 | 3.9 |
| L205Y + Y242F + E259G + N279S + K280Y + S302Y + E390A + G475K | | |
| H1* + N3D + G7A + D118T + W140Y + R180* + S181* + T182S + E189P + N194F + | 5.6 | 4.1 |
| L205Y + Y242F + E259G + N279S + K280Y + S302T + E390A + G475K | | |
| H1* + G7A + D118T + S132D + W140Y + R180* + S181* + T182S + E189P + | 4.9 | 3.5 |
| N194F + L205Y + Y242F + E259G + N279S + K280Y + G303R + Y362V + E390A + | | |
| N474L | | |
| H1* + G7A + D118T + W140Y + R180* + S181* + T182S + E189P + N194F + | 5.9 | 4.3 |
| L205Y + Y242F + E259G + N279S + K280Y + S302T + E390A + G475K | | |
| H1* + G7A + D118T + Q138V + W1 40Y + R180* + S181* + T182S + E189P + | 5.3 | 3.8 |
| N194F + L205Y + Y242F + E259G + N279S + K280Y + G303R + E390A + G476A | | |
| H1* + G7A + D118T + W140Y + R180* + S181* + T182S + E189P + N194F + | 5.1 | 3.7 |
| L205Y + Y242F + E259G + N279S + K280Y + S302T + Y362V + E390A + G475K | | |
| H1* + G7A + D118T + W140Y + R180* + S181* + T182S + E189P + N194F + | 4.9 | 3.5 |
| L205Y + Y242F + E259G + N279S + K280Y + G303R + Y362V + E390A + G476A | | |
| H1* + G7A + D118T + W140Y + R180* + S181* + T182S + E189P + N194F + | 4.4 | 3.2 |
| L205Y + Y242F + E259G + M260D + N279S + K280Y + G303R + E390A + N474L | | |
| H1* + G7A + D118T + W140Y + R180* + S181* + T182S + E189P + N194F + | 4.8 | 3.5 |
| L205Y + T224Y + Y242F + E259G + N279S + K280Y + S302Y + E390A + G475K | | |
| H1* + G7A + D118T + W140Y + R180* + S181* + T182S + E189P + N194F + | 4.6 | 3.3 |
| L205Y + Y242F + E259G + N279S + K280Y + S302Y + Y362V + E390A + G475K | | |
| H1* + G7A + D118T + W140Y + R180* + S181* + T182S + E189P + N194F + | 5.4 | 3.9 |
| L205Y + Y242F + E259G + M260W + N279S + K280Y + S302T + E390A + G475K | | |
| H1* + G7A + D118T + W140Y + R180* + S181* + T182S + E189P + N194F + | 5.2 | 3.8 |
| L205Y + T224Y + Y242F + E259G + N279S + K280Y + G303R + E390A + G476A | | |
| H1* + G7A + D118T + W140Y + R180* + S181* + T182S + E189P + N194F + | 4.0 | 2.9 |
| L205Y + Y242F + E259G + M260D + N279S + K280Y + G303R + E390A + N474F | | |
| H1* + G7A + D118T + W140Y + R180* + S181* + T182S + E189P + N194F + | 4.7 | 3.4 |
| L205Y + Y242F + E259G + N279S + K280Y + S302Y + E390A + N474L | | |
| H1* + G7A + D118T + W140Y + R180* + S181* + T182S + E189P + N194F + | 5.9 | 4.3 |
| L205Y + Y242F + E259G + N279S + K280Y + S302Y + E390A + G476H | | |
| H1* + G7A + D118T + W140Y + R180* + S181* + T182S + E189P + N194F + | 5.9 | 4.3 |
| L205Y + Y242F + E259G + M260D + N279S + K280Y + G303R + E390A + H420A + | | |
| G475K | | |
| H1* + G7A + D118T + Y135 E + W140Y + R180* + S181* + T182S + E189P + | 5.1 | 3.7 |
| N194F + L205Y + Y242F + E259G + N279S + K280Y + G303R + N394W + H420A + | | |
| G475K | | |
| H1* + N3D + G7A + D118T + W140Y + R180* + S181* + T182S + E189P + N194F + | 4.9 | 3.6 |
| L205Y + Y242F + E259G + N279S + K280Y + G303R + N394D + H420A + G475K | | |
| H1* + G7A + D118T + Q138 V + W140Y + R180* + S181* + T182S + E189P + | 5.5 | 4.0 |
| N194F + L205Y + Y242F + E259G + N279S + K280Y + G303R + E390A + H420Q + | | |
| G475K | | |
| H1* + G7A + R37N + D118T + W140Y + R180* + S181* + T182S + E189P + N194F + | 4.7 | 3.4 |
| L205Y + Y242F + E259G + N279S + K280Y + G303R + E390A + H420P + G476H | | |
| H1* + G7A + R37A + Q98L + D118T + W140Y + R180* + S181* + T182S + E189P + | 5.3 | 3.8 |
| N194F + L205Y + Y242F + E259G + N279S + K280Y + S302Y + E390A + G475K | | |
| H1* + G7A + R37A + D118T + W140Y + R180* + S181* + T182S + E189P + N194F + | 5.5 | 4.0 |
| L205Y + Y242F + E259G + N279S + K280Y + G303R + E390A + H420A + G475K | | |
| H1* + G7A + D118T + W140Y + R180* + S181* + T182S + E189P + N194F + | 4.4 | 3.2 |
| L205Y + Y242F + E259G + N279S + K280Y + G303R + E390A + H420P + G475K | | |
| H1* + G7A + D118T + W140Y + R180* + S181* + T182S + E189P + N194F + | 5.7 | 4.2 |
| L205Y + Y242F + E259G + N279S + K280Y + G303R + E390A + N470M + G476A | | |
| H1* + G7A + D118T + W140Y + R180* + S181* + T182S + E189P + N194F + | 4.8 | 3.5 |
| L205Y + Y242F + E259G + M260D + N279S + K280Y + G303R + E390A + H420P + | | |
| G476A | | |
| H1* + G7A + D118T + W140Y + R180* + S181* + T182S + E189P + N194F + | 5.2 | 3.8 |
| L205Y + Y242F + E259G + N279S + K280Y + S302T + E390A + H420A + G475K | | |
| H1* + G7A + R37A + D118T + W140Y + R180* + S181* + T182S + E189P + N194F + | 5.9 | 4.3 |
| L205Y + Y242F + E259G + N279S + K280Y + S302Y + E390A + H420A + G475K | | |
| H1* + G7A + D118T + Y135 E + W140Y + R180* + S181* + T182S + E189P + | 5.5 | 4.0 |
| N194F + L205Y + Y242F + E259G + N279S + K280Y + S302T + Y371N + E390A + | | |
| G475K | | |
| H1* + G7A + R37N + D118T + S132D + W140Y + R180* + S181* + T182S + | 4.8 | 3.5 |
| E189P + N194F + L205Y + Y242F + E259G + N279S + K280Y + G303R + E390A + | | |
| N470M + G475K | | |
| H1* + G7A + D118T + W140Y + R180* + S181* + T182S + E189P + N194F + | 4.7 | 3.4 |
| L205Y + Y242F + E259G + N279S + K280Y + G303R + E390A + H420A + G475K | | |
| H1* + G7A + D118T + W140Y + R180* + S181* + T182S + E189P + N194F + | 4.5 | 3.3 |
| L205Y + Y242F + E259G + N279S + K280Y + G303R + H323W + E390A + H420A + | | |
| N474L | | |
| H1* + G7A + D118T + W140Y + R180* + S181* + T182S + E189P + N194F + L205Y + | 5.2 | 3.8 |
| Y242F + E259G + N279S + K280Y + S302T + E390A + G475K | | |
| H1* + G7A + D118T + W140Y + R180* + S181* + T182S + E189P + N194F + L205Y + | 4.3 | 3.2 |
| Y242F + E259G + N279S + K280Y + G303R + N394W + H420P + G475K | | |
| H1* + G7A + D118T + W140Y + R180* + S181* + T182S + E189P + N194F + L205Y + | 5.2 | 3.8 |
| Y242F + E259G + N279S + K280Y + S302T + H323W + E390A + G475K | | |
| H1* + G7A + D118T + W140Y + R180* + S181* + T182S + E189P + N194F + L205Y + | 5.5 | 4.0 |
| Y242F + E259G + N279S + K280Y + S302T + E390A + G475K | | |
| H1* + G7A + D118T + Y135E + W140Y + R180* + S181* + T182S + E189P + | 4.8 | 3.5 |
| N194F + L205Y + Y242F + E259G + N279S + K280Y + G303R + E390A + R414M + | | |
| G476H | | |
| H1* + G7A + R37N + D118T + W140Y + R180* + S181* + T182S + E189P + N194F + | 4.3 | 3.2 |
| L205Y + Y242F + E259G + N279S + K280Y + S302Y + E390A + H420A + G475K | | |
| H1* + G7A + R37A + D118T + W140Y + R180* + S181* + T182S + E189P + N194F + | 5.3 | 3.8 |
| L205Y + Y242F + E259G + N279S + K280Y + S302T + E390A + H420A + G476A | | |
| H1* + G7A + R37N + D118T + W140Y + R180* + S181* + T182S + E189P + N194F + | 5.2 | 3.8 |
| L205Y + Y242F + E259G + N279S + K280Y + S302Y + E390A + H420A + G475K | | |
| H1* + G7A + R37A + D118T + W140Y + R180* + S181* + T182S + E189P + N194F + | 4.7 | 3.4 |
| L205Y + Y242F + E259G + N279S + K280Y + S302Y + E390A + N474L | | |
| H1* + G7A + R37A + D118T + W140Y + R180* + S181* + T182S + E189P + N194F + | 5.3 | 3.8 |
| L205Y + Y242F + E259G + N279S + K280Y + S302Y + E390A + G475K | | |
| H1* + N3D + G7A + R37A + D118T + W140Y + R180* + S181* + T182S + E189P + | 5.2 | 3.8 |
| N194F + L205Y + Y242F + E259G + N279S + K280Y + S302Y + N394D + H420A + | | |
| G475K | | |
| H1* + G7A + R37A + D118T + W140Y + R180* + S181* + T182S + E189P + N194F + | 5.0 | 3.6 |
| L205Y + Y242F + E259G + N279S + K280Y + S302Y + E390A + H420A + G476H | | |
| H1* + G7A + R37A + D118T + W140Y + R180* + S181* + T182S + E189P + N194F + | 4.8 | 3.5 |
| L205Y + Y242F + E259G + N279S + K280Y + S302Y + N394D + H420A + G475K | | |
| H1* + G7A + R37A + D118T + W140Y + R180* + S181* + T182S + E189P + N194F + | 4.4 | 3.2 |
| L205Y + Y242F + E259G + N279S + K280Y + S302Y + E390A + N474L | | |
| H1* + G7A + R37N + D118T + W140Y + R180* + S181* + T182S + E189P + N194F + | 4.9 | 3.5 |
| L205Y + Y242F + E259G + N279S + K280Y + S302T + E390A + H420A + G475K | | |
| H1* + G7A + R37A + D118T + W140Y + R180* + S181* + T182S + E189P + N194F + | 4.9 | 3.6 |
| L205Y + Y242F + E259G + N279S + K280Y + S302Y + E390A + H420A + G475K | | |
| H1* + G7A + R37N + D118T + W140Y + R180* + S181* + T182S + E189P + N194F + | 4.5 | 3.2 |
| L205Y + Y242F + E259G + N279S + K280Y + S302T + E390A + H420A + G475K | | |
| H1* + G7A + R37A + D118T + W140Y + R180* + S181* + T182S + E189P + N194F + | 5.4 | 3.9 |
| L205Y + Y242F + E259G + N279S + K280Y + S302T + E390A + H420A + G475K | | |
| H1* + G7A + D118T + W140Y + R180* + S181* + T182S + E189P + N194F + L205Y + | 5.0 | 3.6 |
| Y242F + E259G + N279S + K280Y + S302T + N394W + H420A + G475K | | |
| H1* + G7A + R37A + D118T + W140Y + R180* + S181* + T182S + E189P + N194F + | 5.1 | 3.7 |
| L205Y + Y242F + E259G + N279S + K280Y + S302Y + N394D + G475K | | |
| H1* + G7A + R37N + D118T + W140Y + R180* + S181* + T182S + E189P + N194F + | 4.8 | 3.5 |
| L205Y + Y242F + E259G + N279S + K280Y + S302Y + E390A + H420A + G476H | | |
| H1* + G7A + R37N + D118T + W140Y + R180* + S181* + T182S + E189P + N194F + | 5.1 | 3.7 |
| L205Y + Y242F + E259G + N279S + K280Y + S302T + N394D + G475K | | |
| H1* + G7A + R37A + D118T + W140Y + R180* + S181* + T182S + E189P + N194F + | 4.6 | 3.4 |
| L205Y + Y242F + E259G + N279S + K280Y + S302T + E390A + G475K | | |
| H1* + G7A + R37N + D118T + W140Y + R180* + S181* + T182S + E189P + N194F + | 5.4 | 3.9 |
| L205Y + Y242F + E259G + N279S + K280Y + S302T + N394D + H420A + G475K | | |
| H1* + N3G + G7A + D118T + W140Y + R180* + S181* + T182S + E189P + N194F + | 4.8 | 3.5 |
| L205Y + Y242F + E259G + N279S + K280Y + S302T + E390A + H420A + G475K | | |
| H1* + N3D + G7A + D118T + W140Y + R180* + S181* + T182S + E189P + N194F + | 5.4 | 3.9 |
| L205Y + Y242F + E259G + N279S + K280Y + S302T + E390A + G475K | | |
| H1* + N3G + G7A + D118T + W140Y + R180* + S181* + T182S + E189P + N194F + | 5.1 | 3.7 |
| L205Y + Y242F + E259G + N279S + K280Y + S302T + E390A + H420A + G475K | | |
| H1* + N3D + G7A + D118T + W140Y + R180* + S181* + T182S + E189P + N194F + | 5.5 | 4.0 |
| L205Y + Y242F + E259G + N279S + K280Y + S302T + E390A + G475K | | |
| H1* + N3D + G7A + D118T + W140Y + R180* + S181* + T182S + E189P + N194F + | 5.5 | 4.0 |
| L205Y + Y242F + E259G + N279S + K280Y + S302T + E390A + G475K | | |
| H1* + N3D + G7A + D118T + W140Y + R180* + S181* + T182S + E189P + N194F + | 4.8 | 3.5 |
| L205Y + Y242F + E259G + N279S + K280Y + S302Y + E390A + N474L | | |
| H1* + N3D + G7A + D118T + W140Y + R180* + S181* + T182S + E189P + N194F + | 5.5 | 4.0 |
| L205Y + Y242F + E259G + N279S + K280Y + S302T + E390A + G475K | | |
| H1* + N3D + G7A + D118T + W140Y + R180* + S181* + T182S + E189P + N194F + | 5.1 | 3.7 |
| L205Y + Y242F + E259G + N279S + K280Y + S302Y + E390A + G475K | | |
| H1* + N3D + G7A + D118T + W140Y + R180* + S181* + T182S + E189P + N194F + | 4.8 | 3.5 |
| L205Y + Y242F + E259G + N279S + K280Y + S302Y + E390A + G475K | | |
| H1* + N3D + G7A + R37N + D118T + W140Y + R180* + S181* + T182S + E189P + | 4.7 | 3.4 |
| N194F + L205Y + Y242F + E259G + N279S + K280Y + G303R + E390A + H420A + | | |
| G475K | | |
| H1* + N3D + G7A + R37N + D118T + W140Y + R180* + S181* + T182S + E189P + | 5.0 | 3.6 |
| N194F + L205Y + Y242F + E259G + N279S + K280Y + G303R + E390A + H420A + | | |
| G475K | | |
| H1* + N3D + G7A + R37A + D118T + W140Y + R180* + S181* + T182S + E189P + | 5.6 | 4.0 |
| N194F + L205Y + Y242F + E259G + N279S + K280Y + G303R + E390A + G475K | | |
| H1* + N3G + G7A + R37N + D118T + W140Y + R180* + S181* + T182S + E189P + | 4.4 | 3.2 |
| N194F + L205Y + Y242F + E259G + N279S + K280Y + G303R + N394W + H420A + | | |
| G475K | | |
| H1* + N3D + G7A + R37N + D118T + W140Y + R180* + S181* + T182S + E189P + | 4.8 | 3.5 |
| N194F + L205Y + Y242F + E259G + N279S + K280Y + G303R + E390A + H420A + | | |
| G475K | | |
| H1* + N3D + G7A + R37N + D118T + W140Y + R180* + S181* + T182S + E189P + | 4.0 | 2.9 |
| N194F + L205Y + Y242F + E259G + N279S + K280Y + G303R + G361M + H420A + | | |
| G475K | | |
| H1* + N3D + G7A + R37A + D118T + W140Y + R180* + S181* + T182S + E189P + | 5.4 | 3.9 |
| N194F + L205Y + Y242F + E259G + N279S + K280Y + G303R + E390A + H420A + | | |
| G475K | | |
| H1* + G7A + R37A + D118T + W140Y + R180* + S181* + T182S + E189P + N194F + | 5.3 | 3.9 |
| L205Y + Y242F + E259G + M260D + N279S + K280Y + G303R + N394W + G475K | | |
| H1* + G7A + R37N + D118T + W140Y + R180* + S181* + T182S + E189P + N194F + | 4.7 | 3.4 |
| L205Y + Y242F + E259G + M260D + N279S + K280Y + G303R + E390A + G475K | | |
| H1* + G7A + R37A + D118T + W140Y + R180* + S181* + T182S + E189P + N194F + | 6.3 | 4.6 |
| L205Y + T224F + Y242F + N279S + K280Y + G303R + E390A + G475K | | |
| H1* + G7A + R37N + D118T + W140Y + R180* + S181* + T182S + E189P + N194F + | 5.4 | 3.9 |
| L205Y + Y242F + E259G + M260D + N279S + K280Y + G303R + N394D + G475K | | |
| H1* + G7A + R37A + D118T + W140Y + R180* + S181* + T182S + E189P + N194F + | 5.1 | 3.7 |
| L205Y + Y242F + E259G + M260D + N279S + K280Y + G303R + N394D + G475K | | |
| H1* + G7A + R37N + D118T + W140Y + R180* + S181* + T182S + E189P + N194F + | 5.0 | 3.6 |
| L205Y + Y242F + E259G + M260D + N279S + K280Y + G303R + N394D + G475K | | |
| H1* + G7A + D118T + W140Y + R180* + S181* + T182S + E189P + N194F + L205Y + | 5.0 | 3.7 |
| Y242F + E259G + M260D + N279S + K280Y + G303R + H420A + G475K | | |
| H1* + G7A + R37A + D118T + W140Y + R180* + S181* + T182S + E189P + N194F + | 5.3 | 3.8 |
| L205Y + Y242F + E259G + M260D + N279S + K280Y + G303R + E390A + G475K | | |
| H1* + G7A + R37N + D118T + W140Y + R180* + S181* + T182S + E189P + N194F + | 5.2 | 3.8 |
| L205Y + T224Y + Y242F + N279S + K280Y + G303R + E390A + G475K | | |
| H1* + G7A + D118T + W140Y + R180* + S181* + T182S + E189P + N194F + L205Y + | 5.0 | 3.6 |
| Y242F + E259G + N279S + K280Y + G303R + N394W + H420A + G475K | | |
| H1* + G7A + D118T + W140Y + R180* + S181* + T182S + E189P + N194F + L205Y + | 4.9 | 3.6 |
| Y242F + E259G + N279S + K280Y + G303R + N394W + H420A + N474L | | |
| H1* + G7A + D118T + W140Y + P146H + R180* + S181* + T182S + E189P + N194F + | 5.9 | 4.3 |
| L205Y + Y242F + E259G + N279S + K280Y + S302T + E390A + G476A | | |
| H1* + G7A + D118T + W140Y + P146H + R180* + S181* + T182S + E189P + N194F + | 4.9 | 3.5 |
| L205Y + Y242F + E259G + N279S + K280Y + S302Y + E390A + N474L | | |
| H1* + G7A + D118T + W140Y + R180* + S181* + T182S + A185Q + E189P + N194F + | 5.4 | 3.9 |
| Y242F + E259G + F266Y + N279S + K280Y + G303R + E390A + G475K | | |
| H1* + G7A + D118T + W140Y + R180* + S181* + T182S + A185Q + E189P + | 4.6 | 3.3 |
| N194F + L205Q + Y242F + E259G + F266Y + N279S + K280Y + G303R + E390A + | | |
| G475K | | |
| H1* + G7A + D118T + W140Y + R180Q + S181* + T182* + A 85Q + E189P + | 5.1 | 3.7 |
| N194F + Y242F + E259G + F266Y + N279S + K280Y + G303R + E390A + G475K | | |
| H1* + G7A + D118T + W140Y + R180* + S181* + T182S + A185Q + E189P + | 5.1 | 3.7 |
| Y242F + S243Q + E259G + F266Y + N279S + K280Y + G303R + E390A + G475K | | |
| H1* + N3G + G7A + V104M + D118T + W140Y + R180* + S181* + T182S + E189P + | 4.7 | 3.4 |
| N194F + L205Y + Y242F + E259G + N279S + K280Y + S302Y + N394W + G475K | | |
| H1* + N3D + G7A + D118T + W140Y + R180* + S181* + T182S + E189P + N194F + | 5.0 | 3.6 |
| L205Y + Y242F + E259G + N279S + K280Y + G303R + E390A + G475K | | |
| H1* + N3G + G7A + D118T + W140Y + R180* + S181* + T182S + E189P + N194F + | 4.6 | 3.4 |
| L205Y + Y242F + E259G + N279S + K280Y + G303R + N394W + H420A + G475K | | |
| H1* + N3D + G7A + D118T + W140Y + R180* + S181* + T182S + E189P + N194F + | 4.9 | 3.6 |
| L205Y + Y242F + E259G + N279S + K280Y + G303R + E390A + H420A + G475K | | |
| H1* + N3D + G7A + D118T + S132D + W140Y + R180* + S181* + T182S + E189P + | 5.3 | 3.8 |
| N194F + L205Y + Y242F + E259G + N279S + K280Y + G303R + E390A + H420A + | | |
| G475K | | |
| H1* + G7A + D118T + W140Y + R180* + S181* + T182S + E189P + N194F + | 5.4 | 4.0 |
| L205Y + Y242F + E259G + N279S + K280Y + G303R + E390A + N470M + G475K | | |
| H1* + G7A + D118T + W140Y + R180* + S181* + T182S + E189P + N194F + | 5.3 | 3.9 |
| L205Y + Y242F + E259G + N279S + K280Y + S302Y + Y371N + E390A + G475K | | |
| H1* + G7A + D118T + W140Y + R180* + S181* + T182S + E189P + N194F + | 5.8 | 4.2 |
| L205Y + Y242F + E259G + N279S + K280Y + S302T + E390A + G475K | | |
| H1* + G7A + D118T + W140Y + R180* + S181* + T182S + E189P + N194F + | 5.5 | 4.0 |
| L205Y + Y242F + E259G + N279S + K280Y + S302Y + E390A + G475K | | |
| H1* + G7A + D118T + Y135E + W140Y + R180* + S181* + T182S + E189P + | 5.1 | 3.7 |
| N194F + L205Y + Y242F + E259G + N279S + K280Y + S302Y + E390A + G475K | | |
| H1* + G7A + D118T + W140Y + R180* + S181* + T182S + E189P + N194F + | 6.0 | 4.3 |
| L205Y + T224F + Y242F + E259G + N279S + K280Y + S302T + E390A + G475K | | |
| H1* + G7A + R37A + D118T + W140Y + R180* + S181* + T182S + E189P + N194F + | 5.2 | 3.8 |
| L205Y + Y242F + E259G + M260D + N279S + K280Y + G303R + E390A + G475K | | |
| H1* + G7A + R37N + D118T + W140Y + R180* + S181* + T182S + E189P + N194F + | 5.1 | 3.7 |
| L205Y + Y242F + E259G + M260D + N279S + K280Y + G303R + E390A + G475K | | |
| H1* + G7A + D118T + W140Y + R180* + S181* + T182S + E189P + N194F + | 5.3 | 3.8 |
| L205Y + Y242F + E259G + M260D + N279S + K280Y + G303R + N394W + G475K | | |
| H1* + G7A + D118T + W140Y + R180* + S181* + T182S + E189P + N194F + | 5.4 | 3.9 |
| L205Y + Y242F + E259G + M260D + N279S + K280Y + G303R + E390A + G475K | | |
| H1* + G7A + R37A + D118T + W140Y + R180* + S181* + T182S + E189P + | 5.4 | 3.9 |
| N194F + L205Y + Y242F + E259G + M260W + N279S + K280Y + G303R + E390A + | | |
| N470M + G475K | | |
| H1* + G7A + R37N + D118T + W140Y + R180* + S181* + T182S + E189P + N194F + | 5.3 | 3.9 |
| L205Y + Y242F + E259G + M260D + N279S + K280Y + G303R + E390A + G475K | | |
| H1* + G7A + D118T + W140Y + R180* + S181* + T182S + E189P + N194F + L205Y + | 5.5 | 4.0 |
| Y242F + E259G + M260D + N279S + K280Y + G303R + E390A + G475K | | |
| H1* + G7A + D118T + W140Y + R180* + S181* + T182S + E189P + N194F + L205Y + | 5.3 | 3.9 |
| Y242F + E259G + M260D + N279S + K280Y + G303R + N394D + G475K | | |
| H1* + G7A + R37N + D118T + W140Y + R180* + S181* + T182S + E189P + N194F + | 5.0 | 3.7 |
| L205Y + Y242F + E259G + M260W + N279S + K280Y + G303R + E390A + H420A + | | |
| G475K | | |
| H1* + G7A + D118T + W140Y + R180* + S181* + T182S + E189P + N194F + L205Y + | 5.7 | 4.1 |
| Y242F + E259G + M260D + N279S + K280Y + G303R + E390A + N470M + G475K | | |
| H1* + G7A + R37A + D118T + W140Y + R180* + S181* + T182S + E189P + N194F + | 5.4 | 3.9 |
| L205Y + Y242F + E259G + M260D + N279S + K280Y + G303R + N394W + G475K | | |
| H1* + G7A + R37A + D118T + W140Y + R180* + S181* + T182S + E189P + N194F + | 5.1 | 3.7 |
| L205Y + Y242F + E259G + N279S + K280Y + G303R + E390A + H420A + G475K | | |
| H1* + G7A + R37A + D118T + W140Y + R180* + S181* + T182S + E189P + N194F + | 5.3 | 3.8 |
| L205Y + Y242F + E259G + M260D + N279S + K280Y + G303R + E390A + G475K | | |
| H1* + G7A + R37A + D118T + W140Y + R180* + S181* + T182S + E189P + N194F + | 5.3 | 3.8 |
| L205Y + T224Y + Y242F + E259G + N279S + K280Y + G303R + N394W + G475K | | |
| H1* + G7A + R37N + D118T + W140Y + R180* + S181* + T182S + E189P + N194F + | 5.3 | 3.8 |
| L205Y + Y242F + E259G + M260D + N279S + K280Y + G303R + N394W + H420A + | | |
| G475K | | |
| H1* + G7A + R37A + D118T + W140Y + R180* + S181* + T182S + E189P + N194F + | 5.6 | 4.1 |
| L205Y + Y242F + E259G + M260D + N279S + K280Y + G303R + E390A + N470M + | | |
| G475K | | |
| H1* + G7A + R37A + D118T + W140Y + R180* + S181* + T182S + E189P + N194F + | 4.9 | 3.6 |
| L205Y + Y242F + T256D + N279S + K280Y + G303R + E390A + G475K | | |
| H1* + G7A + R37A + D118T + W140Y + R180* + S181* + T182S + E189P + N194F + | 4.9 | 3.5 |
| L205Y + Y242F + E259G + F261P + N279S + K280Y + G303R + E390A + G475K | | |
| H1* + G7A + R37A + D118T + W140Y + R180* + S181* + T182S + E189P + N194F + | 5.0 | 3.6 |
| L205Y + Y242F + E259G + M260D + N279S + K280Y + G303R + N394D + H420A + | | |
| G475K | | |
| H1* + G7A + R37A + D118T + W140Y + R180* + S181* + T182S + E189P + N194F + | 5.4 | 3.9 |
| L205Y + Y242F + E259G + M260D + N279S + K280Y + G303R + N394D + G475K | | |
| H1* + G7A + R37N + D118T + W140Y + N174K + R180* + S181* + T182S + E189P + | 4.5 | 3.3 |
| N194F + L205Y + Y242F + E259G + M260D + N279S + K280Y + G303R + N394W + | | |
| G475K | | |
| H1* + G7A + R37A + D118T + W140Y + R180* + S181* + T182S + E189P + N194F + | 5.4 | 3.9 |
| L205Y + T224Y + Y242F + E259G + N279S + K280Y + G303R + E390A + G475K | | |
| H1* + G7A + R37A + D118T + W140Y + R180* + S181* + T182S + E189P + N194F + | 5.8 | 4.2 |
| L205Y + T224Y + Y242F + N279S + K280Y + G303R + E390A + G475K | | |
| H1* + G7A + R37N + D118T + W140Y + R180* + S181* + T182S + E189P + N194F + | 5.6 | 4.0 |
| L205Y + Y242F + E259G + M260D + N279S + K280Y + G303R + H420A + G475K | | |
| H1* + N3D + G7A + D118T + W140Y + R180* + S181* + T182S + E189P + N194F + | 4.9 | 3.6 |
| L205Y + Y242F + E259G + M260D + N279S + K280Y + G303R + E390A + G475K | | |
| H1* + N3D + G7A + D118T + W140Y + R180* + S181* + T182S + E189P + N194F + | 4.9 | 3.6 |
| L205Y + T224Y + Y242F + E259G + N279S + K280Y + G303R + E390A + G475K | | |
| H1* + N3D + G7A + D118T + W140Y + R180* + S181* + T182S + E189P + N194F + | 5.1 | 3.7 |
| L205Y + T224Y + Y242F + E259G + N279S + K280Y + G303R + E390A + G475K | | |
| H1* + N3D + G7A + D118T + W140Y + R180* + S181* + T182S + E189P + N194F + | 5.5 | 4.0 |
| L205Y + T224Y + Y242F + E259G + N279S + K280Y + G303R + E390A + G475K | | |
| H1* + N3G + G7A + R37N + D118T + W140Y + R180* + S181* + T182S + E189P + | 4.0 | 2.9 |
| N194F + L205Y + Y242F + E259G + N279S + K280Y + G303R + E390A + G475K | | |
| H1* + N3D + G7A + D118T + W140Y + R180* + S181* + T182S + E189P + N194F + | 5.2 | 3.8 |
| L205Y + Y242F + T256D + N279S + K280Y + G303R + E390A + G475K | | |
| H1* + N3D + G7A + D118T + W140Y + R180* + S181* + T182S + E189P + N194F + | 5.0 | 3.7 |
| L205Y + Y242F + T256D + N279S + K280Y + G303R + E390A + G475K | | |
| H1* + N3D + G7A + D118T + W140Y + R180* + S181* + T182S + E189P + N194F + | 5.2 | 3.8 |
| L205Y + Y242F + T256D + N279S + K280Y + G303R + E390A + G475K | | |
| H1* + N3D + G7A + D118T + W140Y + R180* + S181* + T182S + E189P + N194F + | 4.8 | 3.5 |
| L205Y + Y242F + T256D + N279S + K280Y + G303R + E390A + G475K | | |
| H1* + G7A + D118T + W140Y + R180* + S181* + T182S + E189P + N194F + | 6.0 | 4.4 |
| L205Y + Y242F + E259G + N279S + K280Y + S302T + E390A + N470M + G475K | | |
| H1* + G7A + D118T + W140Y + R180* + S181* + T182S + E189P + N194F + | 4.8 | 3.5 |
| L205Y + T224F + Y242F + E259G + N279S + K280Y + S302Y + E390A + G475K | | |
| H1* + G7A + D118T + S132L + W140Y + R180* + S181* + T182S + E189P + | 5.2 | 3.8 |
| N194F + L205Y + Y242F + E259G + N279S + K280Y + S302Y + E390A + G475K | | |
| H1* + G7A + D118T + W140Y + R180* + S181* + T182S + E189P + N194F + | 5.2 | 3.8 |
| L205Y + T224Y + Y242F + E259G + N279S + K280Y + S302T + E390A + G475K | | |
| H1* + G7A + D118T + W140Y + R180* + S181* + T182S + E189P + N194F + | 5.6 | 4.1 |
| L205Y + Y242F + E259G + N279S + K280Y + G303R + E390A + H420A + G476A | | |
| H1* + G7A + D118T + W140Y + R180* + S181* + T182S + E189P + N194F + | 5.2 | 3.8 |
| L205Y + T224F + Y242F + E259G + N279S + K280Y + S302Y + E390A + G475K | | |
| H1* + G7A + D118T + W140Y + R180* + S181* + T182S + E189P + N194F + | 4.9 | 3.5 |
| L205Y + T224Y + Y242F + E259G + N279S + K280Y + S302Y + E390A + G475K | | |
| H1* + G7A + D118T + W140Y + R180* + S181* + T182S + E189P + N194F + | 5.3 | 3.8 |
| L205Y + Y242F + E259G + N279S + K280Y + S302Y + N394D + G475K | | |
| H1* + G7A + D118T + W140Y + R180* + S181* + T182S + E189P + N194F + | 5.4 | 3.9 |
| L205Y + T224F + Y242F + E259G + N279S + K280Y + G303R + E390A + H420A + | | |
| G475K | | |
| H1* + G7A + D118T + W140Y + R180* + S181* + T182S + E189P + N194F + | 5.2 | 3.8 |
| L205Y + T224F + Y242F + E259G + N279S + K280Y + S302Y + E390A + G475K | | |
| H1* + G7A + D118T + W140Y + R180* + S181* + T182S + E189P + N194F + | 5.0 | 3.6 |
| L205Y + T224Y + Y242F + E259G + N279S + K280Y + S302T + E390A + G475K | | |
| H1* + G7A + D118T + W140Y + R180* + S181* + T182S + E189P + N194F + | 5.6 | 4.1 |
| L205Y + Y242F + E259G + N279S + K280Y + S302Y + E390A + G475K | | |
| H1* + G7A + D118T + S132D + W140Y + R180* + S181* + T182S + E189P + | 5.8 | 4.2 |
| N194F + L205Y + Y242F + E259G + N279S + K280Y + S302T + E390A + G475K | | |
| H1* + G7A + D118T + W140Y + R180* + S181* + T182S + E189P + N194F + | 6.0 | 4.4 |
| L205Y + Y242F + E259G + N279S + K280Y + S302T + N394W + G475K | | |
| H1* + G7A + D118T + S132L + W140Y + R180* + S181* + T182S + E189P + N194F + | 5.4 | 3.9 |
| L205Y + T224F + Y242F + E259G + N279S + K280Y + S302Y + E390A + G475K | | |
| H1* + G7A + D118T + Q138V + W1 40Y + R180* + S181* + T182S + E189P + | 5.5 | 4.0 |
| N194F + L205Y + Y242F + E259G + N279S + K280Y + S302Y + N394D + G475K | | |
| H1* + G7A + D118T + S132L + W140Y + R180* + S181* + T182S + E189P + N194F + | 5.3 | 3.8 |
| L205Y + Y242F + E259G + N279S + K280Y + S302Y + N394W + G475K | | |
| H1* + N3D + G7A + D118T + W140Y + R180* + S181* + T182S + E189P + N194F + | 5.9 | 4.3 |
| L205Y + Y242F + E259G + N279S + K280Y + S302T + E390A + G475K | | |
| H1* + G7A + D118T + W140Y + R180* + S181* + T182S + E189P + N194F + | 5.2 | 3.7 |
| L205Y + Y242F + E259G + N279S + K280Y + G303R + E390A + H420A + G476H | | |
| H1* + G7A + D118T + W140Y + R180* + S181* + T182S + E189P + N194F + | 5.4 | 3.9 |
| L205Y + T224F + Y242F + E259G + N279S + K280Y + S302T + E390A + G475K | | |
| H1* + G7A + D118T + W140Y + R180* + S181* + T182S + E189P + N194F + | 5.8 | 4.2 |
| L205Y + Y242F + E259G + N279S + K280Y + S302T + N394D + G475K | | |
| H1* + G7A + D118T + Q138V + W1 40Y + R180* + S181* + T182S + E189P + | 5.6 | 4.1 |
| N194F + L205Y + Y242F + E259G + N279S + K280Y + S302Y + E390A + G475K | | |
| H1* + G7A + D118T + Q138V + W140Y + R180* + S181* + T182S + E189P + | 5.6 | 4.1 |
| N194F + L205Y + Y242F + E259G + N279S + K280Y + S302Y + E390A + G475K | | |
| H1* + G7A + D118T + S132L + W140Y + R180* + S181* + T182S + E189P + | 5.1 | 3.7 |
| N194F + L205Y + Y242F + E259G + N279S + K280Y + S302Y + N394W + G475K | | |
| H1* + G7A + D118T + Q138V + W1 40Y + R180* + S181* + T182S + E189P + | 5.5 | 4.0 |
| N194F + L205Y + T224F + Y242F + E259G + N279S + K280Y + S302Y + E390A + | | |
| G475K | | |
| H1* + G7A + Q98L + D118T + W140Y + R180* + S181* + T182S + E189P + | 5.2 | 3.8 |
| N194F + L205Y + Y242F + E259G + N279S + K280Y + G303R + E390A + G475K | | |
| H1* + G7A + R37N + D118T + W140Y + R180* + S181* + T182S + E189P + N194F + | 4.3 | 3.1 |
| L205Y + Y242F + T256D + E259G + N279S + K280Y + G303R + E390A + G475K | | |
| H1* + G7A + R37A + D118T + W140Y + R180* + S181* + T182S + E189P + N194F + | 3.5 | 2.5 |
| L205Y + Y242F + E259G + N279S + K280Y + G303R + E390A + G475K | | |
| H1* + G7A + R37N + D118T + W140Y + R180* + S181* + T182S + E189P + N194F + | 5.1 | 3.7 |
| L205Y + Y242F + E259G + M260D + N279S + K280Y + G303R + N394D + G475K | | |
| N3G + G7A + R37V + D118T + W140M + R180* + S181* + T182S + E189P + | 2.4 | 1.8 |
| N194F + L205Y + Y242F + E259G + N279S + K280Y + G303R + E390A + H420A + | | |
| G475K | | |
| H1* + G7A + R37N + D118T + W140Y + R180* + S181* + T182S + E189P + N194F + | 5.5 | 4.0 |
| L205Y + Y242F + E259G + N279S + K280Y + G303R + H323W + N394D + G475K | | |
| H1* + G7A + R37A + D118T + W140Y + R180* + S181* + T182S + E189P + N194F + | 4.7 | 3.4 |
| L205Y + Y242F + E259G + N279S + K280Y + G303R + P321Y + L354V + N394D + | | |
| G475K | | |
| H1* + G7A + R37N + D118T + W140Y + R180* + S181* + T182S + E189P + N194F + | 4.9 | 3.5 |
| L205Y + Y242F + E259G + N279S + K280Y + G303R + H323W + Q360M + E390A + | | |
| G475K | | |
| H1* + N3D + G7A + D118T + W140Y + R180* + S181* + T182S + E189P + N194F + | 5.0 | 3.6 |
| L205Y + Y242F + E259G + N279S + K280Y + G303R + H323W + E390A + G475K | | |
| H1* + N3D + G7A + D118T + W140Y + R180* + S181* + T182S + E189P + N194F + | 4.4 | 3.2 |
| L205Y + Y242F + E259G + N279S + K280Y + G303R + H323W + E390A + G475K | | |
| H1* + G7A + D118T + W140Y + R180* + S181* + T182S + A185D + E189P + | 6.5 | 4.7 |
| Y242F + E259G + F266Y + N279S + K280Y + G303R + E390A + G475K | | |
| H1* + G7A + D118T + W140Y + R180Q + S181* + T182* + A185Q + E189P + | 6.5 | 4.8 |
| Y242F + E259G + F266Y + N279S + K280Y + G303R + E390A + G475K | | |
| H1* + G7A + D118T + W140Y + R180* + S181* + T182S + E189P + N194F + | 6.3 | 4.6 |
| Y242F + E259G + N279S + K280Y + S302T + E390A + G475K | | |
| H1* + G7A + D118T + W140Y + R180* + S181* + T182S + E189P + N194F + | 6.2 | 4.5 |
| Y242F + E259G + M260D + N279S + K280Y + S302T + E390A + G475K | | |
| H1* + G7A + R37N + D118T + W140Y + R180* + S181* + T182S + E189P + | 6.2 | 4.5 |
| N194F + Y242F + E259G + N279S + K280Y + S302Y + E390A + G475K | | |
| H1* + G7A + D118T + W140Y + R180* + S181* + T182S + E189P + N194F + | 5.8 | 4.2 |
| Y242F + E259G + M260W + N279S + K280Y + G303R + E390A + G476A | | |
| H1* + G7A + W140Y + R180* + S181* + E189P + N194F + Y242F + E259G + | 6.5 | 4.7 |
| F266Y + N279S + K280Y + S302T + E390A + G475K | | |
| H1* + G7A + D118T + W140Y + R180* + S181* + T182D + E189P + N194F + | 6.2 | 4.5 |
| Y242F + E259G + N279S + K280Y + G303R + E390A + G476A | | |
| H1* + G7A + D118T + S132L + W140Y + R180* + S181* + T182D + E189P + | 6.5 | 4.7 |
| N194F + L205Y + Y242F + E259G + M260D + N279S + K280Y + G303R + E390A + | | |
| G475K | | |
| H1* + G7A + D118T + W140Y + R180* + S181* + T182D + E189P + N194F + | 6.0 | 4.4 |
| Y242F + E259G + M260D + N279S + K280Y + S302T + E390A + G475K | | |
| H1* + N3D + G7A + D118T + W140Y + R180* + S181* + T182D + E189P + N194F + | 6.1 | 4.5 |
| Y242F + E259G + N279S + K280Y + S302T + E390A + H420A + G475K | | |
| H1* + G7A + R37N + D118T + W140Y + R180* + S181* + T182D + E189P + N194F + | 6.0 | 4.4 |
| Y242F + E259G + N279S + K280Y + S302Y + E390A + G475K | | |
| H1* + G7A + D118T + W140Y + R180* + S181* + T182S + A185Q + E189P + | 5.7 | 4.1 |
| N194F + L205Y + T224Y + Y242F + N279S + K280Y + G303R + E390A + G476A | | |
| H1* + G7A + D118T + W140Y + R180* + S181* + T182S + A185Q + E189P + | 6.7 | 4.9 |
| N194F + Y242F + E259G + M260D + N279S + K280Y + S302Y + E390A + N470M + | | |
| G475K | | |
| H1* + G7A + D118T + W140Y + R180* + S181* + T182S + A185Q + E189P + | 6.2 | 4.5 |
| N194F + Y242F + E259G + N279S + K280Y + G303R + E390A + G476A | | |
| H1* + G7A + D118T + W140Y + R180* + S181* + T182S + A185Q + E189P + | 6.7 | 4.9 |
| N194F + Y242F + E259G + M260D + N279S + K280Y + S302T + E390A + G475K | | |
| H1* + N3D + G7A + D118T + W140Y + R180* + S181* + T182S + A185Q + E189P + | 5.1 | 3.7 |
| N194F + Y242F + E259G + N279S + K280Y + S302T + E390A + H420A + G475K | | |
| H1* + G7A + R37N + D118T + W140Y + R180* + S181* + T182S + A185Q + E189P + | 6.4 | 4.6 |
| N194F + Y242F + E259G + N279S + K280Y + S302Y + E390A + G475K | | |
| H1* + G7A + D118T + W140Y + R180* + S181* + T182S + A185Q + E189P + | 5.7 | 4.1 |
| N194F + L205Y + Y242F + E259G + M260W + N279S + K280Y + G303R + E390A + | | |
| G476A | | |
| H1* + G7A + D118T + W140Y + R180* + S181* + T182S + E189P + N194F + | 6.0 | 4.4 |
| T224Y + Y242F + F266Y + N279S + K280Y + G303R + E390A + G476A | | |
| H1* + G7A + D118T + W140Y + R180* + S181* + T182S + E189P + N194F + | 6.1 | 4.4 |
| Y242F + E259G + M260D + F266Y + N279S + K280Y + S302Y + E390A + N470M + | | |
| G475K | | |
| H1* + G7A + D118T + W140Y + R180* + S181* + T182S + E189P + N194F + | 6.1 | 4.4 |
| Y242F + E259G + F266Y + N279S + K280Y + S302T + E390A + G475K | | |
| H1* + G7A + D118T + W140Y + R180* + S181* + T182S + E189P + N194F + | 5.8 | 4.2 |
| Y242F + E259G + F266Y + N279S + K280Y + G303R + E390A + G476A | | |
| H1* + G7A + D118T + S132L + W140Y + R180* + S181* + T182S + E189P + N194F + | 5.9 | 4.3 |
| Y242F + E259G + M260D + F266Y + N279S + K280Y + G303R + E390A + G475K | | |
| H1* + G7A + D118T + W140Y + R180* + S181* + T182S + E189P + N194F + | 6.1 | 4.4 |
| Y242F + E259G + M260D + F266Y + N279S + K280Y + S302T + E390A + G475K | | |
| H1* + G7A + D118T + W140Y + R180* + S181* + T182S + E189P + N194F + | 5.6 | 4.1 |
| Y242F + E259G + M260W + F266Y + N279S + K280Y + G303R + E390A + G476A | | |
| H1* + G7A + D118T + W140Y + R180* + S181* + T182 D + E189P + N194F + | 5.6 | 4.1 |
| Y242F + E259G + F266Y + N279S + K280Y + S302T + E390A + G475K | | |
| H1* + G7A + D118T + W140Y + R180* + S181* + T182D + E189P + N194F + | 5.5 | 4.0 |
| Y242F + E259G + M260D + F266Y + N279S + K280Y + S302T + E390A + G475K | | |
| H1* + N3D + G7A + D118T + W140Y + R180* + S181* + T182D + E189P + N194F + | 6.5 | 4.7 |
| Y242F + E259G + F266Y + N279S + K280Y + S302T + E390A + H420A + G475K | | |
| H1* + G7A + R37N + D118T + W140Y + R180* + S181* + T182D + E189P + N194F + | 6.4 | 4.7 |
| Y242F + E259G + F266Y + N279S + K280Y + S302Y + E390A + G475K | | |
| H1* + G7A + D118T + W140Y + R180* + S181* + T182S + A185Q + E189P + | 6.5 | 4.7 |
| N194F + T224Y + Y242F + F266Y + N279S + K280Y + G303R + E390A + G476A | | |
| H1* + G7A + D118T + W140Y + R180* + S181* + T182S + A185Q + E189P + | 6.6 | 4.8 |
| N194F + Y242F + E259G + F266Y + N279S + K280Y + S302T + E390A + G475K | | |
| H1* + G7A + D118T + W140Y + R180* + S181* + T182S + A185Q + E189P + | 6.7 | 4.9 |
| N194F + Y242F + E259G + F266Y + N279S + K280Y + G303R + E390A + G476A | | |
| H1* + G7A + D118T + S132L + W140Y + R180* + S181* + T182S + A185Q + | 6.2 | 4.5 |
| E189P + N194F + L205Y + Y242F + E259G + M260D + F266Y + N279S + K280Y + | | |
| G303R + E390A + G475K | | |
| H1* + N3D + G7A + D118T + W140Y + R180* + S181* + T182S + A185Q + E189P + | 6.7 | 4.9 |
| N194F + Y242F + E259G + F266Y + N279S + K280Y + S302T + E390A + H420A + | | |
| G475K | | |
| H1* + G7A + D118T + W140Y + R180* + S181* + T182S + A185Q + E189P + | 7.0 | 5.1 |
| N194F + Y242F + E259G + M260W + F266Y + N279S + K280Y + G303R + E390A + | | |
| G476A | | |
| H1* + G7A + W140Y + R180* + S181* + E189P + N194F + Y242F + E259G + | 5.5 | 4.0 |
| M260D + N279S + K280Y + S302T + E390A + G475K | | |
| H1* + N3D + G7A + W140Y + R180* + S181* + E189P + N194F + Y242F + E259G + | 6.0 | 4.3 |
| F266Y + N279S + K280Y + S302T + E390A + H420A + G475K | | |
| H1* + G7A + R37N + W140Y + R180* + S181* + E189P + N194F + Y242F + E259G + | 5.6 | 4.1 |
| F266Y + N279S + K280Y + S302Y + E390A + G475K | | |
| H1* + G7A + W140Y + R180* + S181* + E189P + N194F + Y242F + E259G + F266Y + | 7.1 | 5.1 |
| N279S + K280Y + G303R + E390A + G476A | | |
| H1* + G7A + D118T + W140Y + R180* + S181* + T182S + E189P + N194F + | 6.7 | 4.9 |
| Y242F + E259G + M260D + N279S + K280Y + S302Y + E390A + N470M + G475K | | |
| H1* + G7A + D118T + W140Y + R180* + S181* + T182D + E189P + N194F + | 6.2 | 4.5 |
| Y242F + E259G + M260D + N279S + K280Y + S302Y + E390A + N470M + G475K | | |
| H1* + G7A + D118T + W140Y + R180* + S181* + T182D + E189P + N194F + | 6.4 | 4.7 |
| Y242F + E259G + M260W + N279S + K280Y + G303R + E390A + G476A | | |
| H1* + G7A + D118T + W140Y + R180* + S181* + T182S + A185Q + E189P + | 6.4 | 4.7 |
| N194F + Y242F + E259G + M260W + N279S + K280Y + G303R + E390A + G476A | | |
| H1* + G7A + D118T + W140Y + R180* + S181* + T182S + E189P + N194F + | 6.9 | 5.0 |
| T224Y + Y242F + N279S + K280Y + G303R + E390A + G476A | | |
| H1* + G7A + D118T + W140Y + R180* + S181* + T182D + E189P + N194F + | 6.5 | 4.7 |
| T224Y + Y242F + N279S + K280Y + G303R + E390A + G476A | | |
| H1* + G7A + D118T + W14OY + R180* + S181* + T182S + A185Q + E189P + | 6.8 | 4.9 |
| N194F + T224Y + Y242F + N279S + K280Y + G303R + E390A + G476A | | |
| H1* + G7A + D118T + W140Y + R180* + S181* + T182S + A185Q + E189P + | 7.0 | 5.1 |
| N194F + Y242F + E259G + N279S + K280Y + S302T + E390A + G475K | | |
| H1* + G7A + D118T + W140Y + R180* + S181* + T182D + E189P + N194F + | 6.7 | 4.9 |
| Y242F + E259G + F266Y + N279S + K280Y + G303R + E390A + G476A | | |
| H1* + G7A + D118T + W140Y + R180* + S181* + T182D + E189P + N194F + | 6.4 | 4.7 |
| Y242F + E259G + M260D + N279S + K280Y + S302Y + E390A + N470M + G475K | | |
| H1* + G7A + D118T + W140Y + R180* + S181* + T182D + A185Q + E189P + | 6.1 | 4.5 |
| N194F + Y242F + E259G + F266Y + N279S + K280Y + G303R + E390A + G475K | | |
| H1* + G7A + D118T + W140Y + R180* + S181* + T182D + A185Q + Y242F + | 5.9 | 4.3 |
| S243Q + E259G + F266Y + N279S + G303R + E390A + G475K | | |
| H1* + G7A + D118T + W140Y + R180* + S181* + T182 D + N194F + Y242F + | 6.4 | 4.6 |
| S243Q + E259G + F266Y + N279S + G303R + E390A + G475K | | |
| H1* + G7A + D118T + W140Y + R180* + S181* + T182S + A185Q + E189P + | 5.3 | 3.8 |
| N196I + Y242F + E259G + F266Y + N279S + K280Y + G303R + E390A + G475K | | |
| H1* + G7A + D118T + W140Y + R180* + S181* + T182S + A185D + E189P + | 6.5 | 4.7 |
| Y242F + E259G + F266Y + N279S + K280Y + G303R + E390A + H420Y + G475K | | |
| H1* + G7A + D118T + W140Y + R180* + S181* + A 185Q + E189P + N194F + | 5.8 | 4.2 |
| L205Y + Y242F + E259G + F266Y + N279S + K280Y + G303R + E390A + G475K | | |
| H1* + G7A + W140Y + R180* + S181* + E189P + N194F + Y242F + E259G + | 6.1 | 4.4 |
| M260W + N279S + K280Y + G303R + E390A + G476A | | |
| H1* + G7A + D118T + W140Y + R180* + S181* + T182S + E189P + N194F + | 7.0 | 5.1 |
| Y242F + E259G + M260D + N279S + K280Y + S302Y + E390A + N470M + G475K | | |
| H1* + G7A + D118T + W140Y + R180* + S181* + T182D + E189P + N194F + | 6.2 | 4.5 |
| T224Y + Y242F + N279S + K280Y + G303R + E390A + G476A | | |
| H1* + G7A + D118T + W140Y + R180* + S181* + T182S + E189P + N194F + | 7.0 | 5.1 |
| T224Y + Y242F + N279S + K280Y + G303R + E390A + G476A | | |
| H1* + G7A + D118T + W140Y + R180* + S181* + T182 D + A185Q + E189P + | 6.3 | 4.6 |
| N194F + T224Y + Y242F + N279S + K280Y + G303R + E390A + G476A | | |
| H1* + G7A + D118T + W140Y + R180* + S181* + T182D + A185Q + N194F + | 7.1 | 5.2 |
| Y242F + E259G + N279S + K280Y + S302T + E390A + G475K | | |
| H1* + G7A + R37N + D118T + W140Y + R180* + S181* + T182S + A185Q + E189P + | 6.1 | 4.4 |
| N194F + L205Y + Y242F + E259G + N279S + K280Y + S302Y + E390A + G475K | | |
| H1* + G7A + D118T + W140Y + R180* + S181* + T182S + A185Q + E189P + N194F + | 6.8 | 5.0 |
| L205Y + Y242F + E259G + M260D + N279S + K280Y + S302T + E390A + G475K | | |
| H1* + N3D + G7A + D118T + W140Y + R180* + S181* + T182D + E189P + N194F + | 6.2 | 4.5 |
| L205Y + Y242F + E259G + N279S + K280Y + S302T + E390A + H420A + G475K | | |
| H1* + G7A + W140Y + R180* + S181* + E189P + N194F + Y242F + E259G + | 6.9 | 5.0 |
| M260D + N279S + K280Y + S302Y + E390A + N470A + G475K | | |
| H1* + G7A + S132L + W140Y + R180* + S181* + E189P + N194F + Y242F + | 6.8 | 4.9 |
| E259G + M260D + N279S + K280Y + G303R + E390A + G475K | | |
| H1* + G7A + A31S + R37N + D118T + W140Y + R180* + S181* + T182S + A185Q + | 6.4 | 4.7 |
| E189P + N194F + Y242F + E259G + F266Y + N279S + K280Y + S302Y + E390A + | | |
| G475K | | |
| H1* + G7A + D118T + W140Y + R180* + S181* + T182S + A185Q + E189P + | 6.7 | 4.9 |
| N194F + Y242F + E259G + M260W + N279S + K280Y + G303R + E390A + G476A | | |
| H1* + G7A + D118T + W140Y + R180* + S181* + T182D + E189P + N194F + | 6.5 | 4.7 |
| L205Y + T224Y + Y242F + F266Y + N279S + K280Y + G303R + E390A + G476A | | |
| H1* + G7A + D118T + W140Y + R180* + S181* + T182S + A185Q + E189P + | 6.7 | 4.9 |
| N194F + Y242F + E259G + N279S + K280Y + S302T + E390A + G475K | | |
| R180* + S181* | 1.7 | — |
| F116L + D123N + N126D + T131I + R180* + S181* + F202Y | 1.1 | — |
| HV + N54S + V56T + K72R + G113Q + F116Q + D118Q + D123N + N126D + T131I + | 1.2 | — |
| K142G + D144N + W167F + K172G + N174S + S181T + T182* + G183* + F202Y + | | |
| E390A + F395Y + K457R + S472R + V473C + G475K | | |
| T40N + F116L + D123N + N126D + N128Y + T131I + F155W + R180* + S181* + | 1.6 | — |
| T182D + E189P + F202Y + K457N +P458S + G475K + G476E | | |
| H1* + N54S + V56T + K72R + N106D + A109G + G113Q + F116Q + D118Q + | 1.2 | — |
| D123N + N126D + T131I + K142G + D144N + W167F + K172G + N174S + S181T + | | |
| T182* + G183* + F202Y + E390A + F395Y + K457R + S472R + V473C + G475K | | |
| A60V + Q98L + G110K + R180* + S181* + A383D | 3.3 | 2.4 |
| Q98L + G110K + S132D + Y135E + R180* + S181* + T315A | 3.2 | 2.4 |
| G7H + Q98L + G110K + R180* + S181* | 4.3 | 3.1 |
| G7H + W167P + R180* + S181* + G455R | 2.3 | — |
| Q98L + V103A + G110K + R180* + S181* | 2.8 | 2.0 |
| E169I + R180* + S181* | 1.4 | — |
| T114K + R180* + S181* | 1.5 | — |
| W167A + R180* + S181* | 1.4 | — |
| E169V + R180* + S181* | 1.8 | — |
| R180* + S181* + W188C | 1.6 | — |
| W167D + R180* + S181* | 2.1 | 1.5 |
| Y177D + R180* + S181* | 1.3 | — |
| W167S + R180* + S181* | 1.8 | — |
| W167E + R180* + S181* | 1.6 | — |
| W167P + R180* + S181* | 1.6 | — |
| R180* + S181* + E193I + H323N | 1.7 | — |
| R180* + S181* + L205R | 1.1 | — |
| R180* + S181* + A203F | 1.6 | — |
| W159L + R180* + S181* | 1.5 | — |
| Y160P + R180* + S181* | 1.4 | — |
| W159H + R180* + S181* | 1.5 | — |
| K178A + R180* + S181* | 1.5 | — |
| R180* + S181* + G183P | 1.3 | — |
| Q136T + R180* + S181* | 1.4 | — |
| K142P + R180* + S181* | 1.4 | — |
| K142E + R180* + S181* | 1.4 | — |
| Y152L + R180* + S181* | 1.3 | — |
| A111L + R180* + S181* | 1.7 | — |
| G110K + R180* + S181* | 1.8 | — |
| A111G + R180* + S181* | 1.5 | — |
| V117G + R180* + S181* | 1.3 | — |

### Example 5: Wash Performance of Alpha-Amylases Using Automatic Mechanical Stress Assay

In order to assess the wash performance of the alpha-amylases in a detergent base composition, washing experiments may be performed using Automatic Mechanical Stress Assay (AMSA). With the AMSA test the wash performance of a large quantity of small volume enzyme-detergent solutions can be examined. The AMSA plate has a number of slots for test solutions and a lid firmly squeezing the textile swatch to be washed against all the slot openings. During the washing time, the plate, test solutions, textile and lid are vigorously shaken to bring the test solution in contact with the textile and apply mechanical stress in a regular, periodic oscillating manner. For further description see WO 02/42740, especially the paragraph “Special method embodiments” at page 23-24.

### General Wash Performance Description

A test solution comprising water and model detergent A as described below, and the enzyme of the invention at concentration of 0 or 0.09 ppm, is prepared. Fabrics stained with starch (CS-28 from Center For Test materials BV, P.O. Box 120, 3133 KT, Vlaardingen, The Netherlands) is added and washed for 10 minutes at 20° C. After thorough rinse under running tap water and drying in the dark, the light intensity values of the stained fabrics are subsequently measured as a measure for wash performance. The test with 0 mg enzyme protein/L is used as a blank and corresponds to the contribution from the detergent. Preferably mechanical action is applied during the wash step, e.g. in the form of shaking, rotating or stirring the wash solution with the fabrics. The AMSA wash performance experiments may be conducted under the experimental conditions specified below:

TABLE A
Experimental condition

| | Liquid Model detergent A |
|---|---|
| Detergent | (see Table B) |
| Detergent dosage | 3.33 g/L |
| Test solution volume | 160 micro L |
| pH | As is |
| Wash time | 10 minutes |
| Temperature | 20° C. |
| Water hardness | 15° dH |
| Enzyme concentration in test | 0.09 pmm |
| Test material | CS-28 (Rice starch cotton) |

TABLE B
Model detergent A

| | Content of | % active |
|---|---|---|
| | compound | component |
| Compound | (% w/w) | (% w/w) |
| LAS | 12.00 | 11.60 |
| AEOS, SLES | 17.63 | 4.90 |
| Soy fatty acid | 2.75 | 2.48 |
| Coco fatty acid | 2.75 | 2.80 |
| AEO | 11.00 | 11.00 |
| Sodium hydroxide | 1.75 | 1.80 |
| Ethanol/Propan-2-ol | 3.00 | 2.70/0.30 |
| MPG | 6.00 | 6.00 |
| Glycerol | 1.71 | 1.70 |
| TEA | 3.33 | 3.30 |
| Sodium formate | 1.00 | 1.00 |
| Sodium citrate | 2.00 | 2.00 |
| DTMPA | 0.48 | 0.20 |
| PCA | 0.46 | 0.18 |
| Phenoxy ethanol | 0.50 | 0.50 |
| H₂O, ion exchanged | 33.64 | 33.64 |

Water hardness was adjusted to 15° dH by addition of CaCl₂, MgCl₂, and NaHCO₃ (Ca²⁺:Mg²⁺:HCO³⁻=4:1:7.5) to the test system. After washing the textiles were flushed in tap water and dried.

The wash performance is measured as the brightness expressed as the intensity of the light reflected from the sample when illuminated with white light. When the sample is stained the intensity of the reflected light is lower, than that of a clean sample. Therefore, the intensity of the reflected light can be used to measure wash performance.

Color measurements are made with a professional flatbed scanner (EPSON Expression 10000XL, EPSON) used to capture an image of the washed textile.

To extract a value for the light intensity from the scanned images, 484→24 Bit Color pixel values from the image are converted into values for red, green and blue (RGB). The intensity value (Int) is calculated by adding the RGB values together as vectors and then taking the length of the resulting vector:

Int=\sqrt{r²+g²+b²}

TABLE 3
Wash performance of the variants according to the invention are shown in the table below.

| Modifications in SEQ ID NO: 1 | IF |
|---|---|
| SEQ ID NO: 1 | 1.0 |
| H1* + G7A + R180* + S181* + T182G + Y242F + E259G + N279S + G303R + E390A + | 1.2 |
| G475K | |
| H1* + G7A + R180* + T182L + G183* + Y242F + E259G + N279S + G303R + E390A + | 1.1 |
| G475K | |
| H1* + G7A + R180* + S181* + N194F + Y242F + E259G + N279S + G303R + E390A + | 1.1 |
| G475K | |
| H1* + G7A + R180* + S181* + T182S + Y242F + E259G + N279S + G303R + E390A + | 1.1 |
| G475K | |
| H1* + G7A + R180* + S181* + L205F + Y242F + E259G + N279S + G303R + E390A + | 1.1 |
| G475K | |
| H1* + G7A + R180* + S181* + A185Q + Y242F + E259G + N279S + G303R + E390A + | 1.1 |
| G475K | |
| H1* + G7A + R180* + S181* + T182D + Y242F + E259G + N279S + G303R + E390A + | 1.1 |
| G475K | |
| H1* + G7A + R180* + S181* + T182E + Y242F + E259G + N279S + G303R + E390A + | 1.1 |
| G475K | |
| H1* + G7A + R180* + S181* + A185E + Y242F + E259G + N279S + G303R + E390A + | 1.1 |
| G475K | |
| H1* + G7A + R180* + S181* + A185D + Y242F + E259G + N279S + G303R + E390A + | 1.1 |
| G475K | |
| H1* + G7A + R180* + S181* + N196F + L205Y + Y242F + E259G + N279S + G303R + | 1.2 |
| E390A + G475K | |
| H1* + G7A + R180* + S181* + E189P + Y242F + E259G + N279S + K280W + G303R + | 1.2 |
| E390A + G475K | |
| H1* + G7A + S181L + T182* + G183* + Y242F + E259G + N279S + G303R + E390A + | 1.2 |
| G475K | |
| H1* + G7A + R180* + S181* + Y242F + E259G + F266Y + N279S + G303R + E390A + | 1.2 |
| G475K | |
| H1* + G7A + R180* + S181* + E189P + Y242F + E259G + N279S + K280Y + G303R + | 1.2 |
| E390A + G475K | |
| H1* + G7A + R180* + S181* + N196F + Y242F + E259G + N279S + G303R + E390A + | 1.1 |
| G475K | |
| H1* + G7A + M105l + R180* + S181* + Y242F + E259G + N279S + G303R + E390A + | 1.1 |
| G475K | |
| H1* + G7A + A60P + R180* + S181* + Y242F + E259G + N279S + G303R + E390A + | 1.1 |
| G475K | |
| H1* + G7A + M105L + R180* + S181* + Y242F + E259G + N279S + G303R + E390A + | 1.1 |
| G475K | |
| H1* + G7A + R180* + S181* + A185R + Y242F + E259G + N279S + G303R + E390A + | 1.2 |
| G475K | |
| H1* + G7A + R180* + S181* + E189P + Y242F + E259G + N279S + G303R + E390A + | 1.1 |
| G475K | |
| H1* + G7A + R180* + S181* + A185K + Y242F + E259G + N279S + G303R + E390A + | 1.1 |
| G475K | |
| H1* + G7A + R180* + S181* + E189P + L205Y + Y242F + E259G + N279S + K280F + | 1.1 |
| G303R + E390A + G475K | |
| H1* + G7A + R180* + S181* + E189P + L205F + Y242F + E259G + N279S + K280F + | 1.1 |
| G303R + E390A + G475K | |
| H1* + G7S + T4ON + Q98L + R180* + S181* + Y242F + E259G + N279S + G303R + | 1.2 |
| Y362V + E390A + G475K | |
| H1* + G7A + R180* + S181* + Y242F + E259G + N279S + W283N + S286K + G303R + | 1.2 |
| H320T + H323A + E390A + G475K | |
| H1* + G7A + R180* + S181* + Y242F + E259G + N279S + G303R + M322G + E359W + | 1.2 |
| Q360W + E390A + G475K | |
| H1* + G7A + R180* + S181* + Y242F + E259G + F261S + N279S + S286K + G303R + | 1.2 |
| Y362V + E390A + G475K | |
| H1* + G7A + D118T + W140Y + R180* + S181* + E189P + N194F + L205Y + Y242F + | 1.3 |
| E259G + N279S + K280Y + G303R + E390A + G475K | |
| H1* + G7A + D118T + W140Y + R180* + S181* + T182S + A185D + E189P + Y242F + | 1.3 |
| E259G + F266Y + N279S + K280Y + G303R + E390A + G475K | |
| H1* + G7A + T40N + Q98L + R180* + S181* + Y242F + E259G + N279S + G303R+ | 1.1 |
| E390A + G475K | |
| H1* + G7A + W140Y + R180* + S181* + E189 P + N194 F + Y242 F + E259G + F266Y + | 1.1 |
| N279S + K280Y + G303R+ E390A + G475K | |
| H1* + G7A + D118T + W140Y + R180* + S181* + T182S + A185Q + E189P + N194F + | 1.1 |
| Y242F + E259G + N279S + K280Y + G303R+ E390A + G476A | |
| H1* + G7A + R180* + S181* + Y242F + E259G + N279S + S286K + G303R+ H323A + | 1.1 |
| E390A + G475K | |
| H1* + G7A + R180* + S181* + Y242F + E259G + N279S + G303R+ Y362V + E390A + | 1.1 |
| G475K | |
| H1* + G7A + R180* + S181* + Y242F + E259G + N279S + G303R+ P321H + Q360Y + | 1.1 |
| E390A + G475K | |
| H1* + G7A + T4ON + Q98L + R180* + S181* + Y242F + E259G + N279S + G303R + | 1.1 |
| E390A + G475K | |
| H1* + G7A + R180* + S181* + Y242F + E259G + N279S + G303R + M322Y + Q360Y + | 1.1 |
| E390A + G475K | |
| H1* + G7A + l137F + R180* + S181* + Y242F + E259G + F261S + N279S + G303R + | 1.1 |
| Y362V + E390A + G475K | |
| H1* + G7A + W140Y + R180* + S181* + E189P + N194F + L205Y + Y242F + E259G + | 1.2 |
| N279S + K280Y + G303R + E390A + G475K | |
| H1* + G7A + R180* + S181* + Y242F + E259G + N279S + G303R + H320T + E390A + | 1.2 |
| G475K | |
| H1* + G7A + R180* + S181* + Y242F + E259G + N279S + W283N + G303R + H320T + | 1.2 |
| H323A + E390A + G475K | |
| H1* + G7A + R180* + S181* + Y242F + E259G + N279S + G303R + H323A + E390A + | 1.2 |
| G475K | |
| H1* + G7A + R180* + S181* + Y242F + E259G + N279S + G303R + M322G + E390A + | 1.2 |
| G475K | |
| H1* + G7A + Q98L + R180* + S181* + Y242F + E259G + N279S + G303R + E390A + | 1.2 |
| G475K | |
| H1* + G7A + R180* + S181* + Y242F + E259G + N279S + G303R + P321H + E390A + | 1.2 |
| G475K | |
| H1* + G7A + D118T + W140Y + R180* + S181* + T182D + N194F + L205Y + Y242F + | 1.2 |
| E259G + N279S + G303R + E390A + G475K | |
| H1* + G7A + Q98L + R180* + S181* + Y242F + E259G + F261 K + N279S + G303R + | 1.1 |
| Y362V + E390A + G475K | |
| H1* + G7A + R180* + S181* + Y242F + E259G + N279S + G303R + Q360Y + E390A + | 1.1 |
| G475K | |
| H1* + G7A + R180* + S181* + Y242F + E259G + F261S + N279S + G303R + Y362V + | 1.1 |
| E390A + G475K | |
| H1* + G7S + T40N + Q98L + R180* + S181* + Y242F + E259G + N279S + G303R + | 1.1 |
| E390A + G475K | |
| H1* + G7A + R180* + S181* + Y242F + E259G + N279S + G303R + M322Y + E390A + | 1.1 |
| G475K | |
| H1* + G7A + R180* + S181* + Y242F + E259G + F261S + N279S + G303R + E390A + | 1.1 |
| G475K | |
| H1* + G7S + T40N + Q98L + R180* + S181* + Y242F + E259G + N279S + G303R + | 1.1 |
| H320T + E390A + G475K | |
| H1* + G7A + W140Y + R180* + S181* + A185Q + N194F + L205Y + Y242F + E259G + | 1.1 |
| N279S + G303R + E390A + G475K | |
| H1* + G7A + W140Y + R180* + S181* + T182S + E189P + N194F + L205Y + Y242F + | 1.2 |
| E259G + N279S + K280Y + G303R + E390A + G475K | |
| H1* + G7A + W140Y + R180* + S181* + T182D + E189P + N194F + Y242F + E259G + | 1.1 |
| N279S + K280Y + G303R + E390A + G475K | |
| H1* + G7A + W140Y + R180* + S181* + T182D + E189P + N194F + L205Y + Y242F + | 1.1 |
| E259G + N279S + K280Y + G303R + E390A + G475K | |
| H1* + G7A + W140Y + R180* + S181* + T182D + E189P + N194F + Y242F + E259G + | 1.1 |
| N279S + K280Y + G303R + E390A + G475K | |
| H1* + G7A + W140Y + R180* + S181* + E189P + N194F + L205V + Y242F + E259G + | 1.1 |
| N279S + K280Y + G303R + E390A + G475K | |
| H1* + G7A + W140Y + R180* + S181* + T182S + E189P + N194F + Y242F + E259G + | 1.1 |
| N279S + K280Y + G303R + E390A + G475K | |
| H1* + G7A + W140Y + R180* + S181* + T182D + A185Q + N194F + L205V + Y242F + | 1.1 |
| E259G + N279S + G303R + E390A + G475K | |
| H1* + G7A + W140Y + R180* + S181* + T182D + N194F + L205V + Y242 F + E259G + | 1.1 |
| F266Y + N279S + G303R + E390A + G475K | |
| H1* + G7A + W140Y + R180* + S181* + E189P + N194F + Y242F + E259G + N279S + | 1.2 |
| K280Y + G303R + E390A + G475K | |
| H1* + G7A + F116N + W140Y + R180* + S181* + T182D + E189P + N194F + L205Y + | 1.2 |
| Y242F + E259G + N279S + K280Y + G303R + E390A + G475K | |
| H1* + G7A + W140Y + R180* + S181* + E189P + N194F + Y242F + E259G + N279S + | 1.2 |
| K280Y + G303R + E390A + G475K | |
| H1* + G7A + W140Y + R180* + S181* + N194F + L205Y + Y242F + E259G + F266Y + | 1.2 |
| N279S + G303R + E390A + G475K | |
| H1* + G7A + D123N + W140Y + l176V + R180* + S181* + N194F + L205V + Y242F + | 1.2 |
| E259G + N279S + G303R + E390A + G475K | |
| H1* + G7A + D123N + W140Y + R180* + S181* + N194F + L205V + Y242F + E259G + | 1.2 |
| N279S + G303R + E390A + G475K | |
| H1* + G7A + F116N + W140Y + R180* + S181* + E189P + N194F + Y242F + E259G + | 1.2 |
| N279S + K280Y + G303R + E390A + G475K | |
| H1* + G7A + D118T + W140Y + R180* + S181* + T182D + E189P + N194F + L205Y + | 1.2 |
| Y242F + E259G + N279S + K280Y + G303R + E390A + G475K | |
| H1* + G7A + D123N + W140Y + R180* + S181* + N194F + L205Y + Y242F + E259G + | 1.2 |
| N279S + G303R + E390A + G475K | |
| H1* + G7A + W140Y + R180* + S181* + T182D + A185Q + Y242F + S243Q + E259G + | 1.1 |
| F266Y + N279S + G303R + E390A + G475K | |
| H1* + G7A + W140Y + R180* + S181* + N194F + L205Y + Y242F + E259G + N279S + | 1.1 |
| G303R + E390A + N422V + G475K | |
| H1* + G7A + W140Y + R180* + S181* + A185Q + Y242F + S243Q + E259G + F266Y + | 1.1 |
| N279S + K280Y + G303R + E390A + G475K | |
| H1* + G7A + W140Y + R180* + S181* + N194F + L205Y + Y242F + E259G + N279S + | 1.1 |
| G303R + E390A + G475E | |
| H1* + G7A + W140Y + R180* + S181* + N194F + L205Y + Y242 F + E259G + F261P + | 1.1 |
| N279S + G303R + E390A + G475K | |
| H1* + G7A + W140Y + R180* + S181* + T182D + E189P + Y242F + S243Q + E259G + | 1.1 |
| N279S + K280Y + G303R + E390A + G475K | |
| H1* + G7A + D118T + W140Y + R180* + S181* + T182S + E189P + N194F + L205Y + | 1.1 |
| Y242F + E259G + N279S + K280Y + G303R + E390A + H420P + G475K | |
| H1* + N3D + G7A + D118T + W140Y + R180* + S181* + T182S + E189P + N194F + | 1.1 |
| L205Y + Y242F + E259G + N279S + K280Y + G303R + E390A + H420A + G475K | |
| H1* + G7A + V103l + D118T + W140Y + R180* + S181* + T182S + E189P + N194F + | 1.2 |
| L205Y + Y242 F + E259G + N279S + K280Y + S302Y + E39OA + N474L | |
| H1* + N3D + G7A + D118T + W140Y + R180* + S181* + T182S + E189P + N194F + | 1.1 |
| L205Y + Y242F + E259G + N279S + K280Y + S302T + E390A + H420A + G475K | |
| H1* + G7A + D118T + W140Y + R180* + S181* + T182S + E189P + N194F + L205Y + | 1.1 |
| Y242F + E259G + N279S + K280Y + S302T + E390A + G475K | |
| H1* + G7A + D118T + W140Y + R180* + S181* + T182S + E189P + N194F + L205Y + | 1.1 |
| Y242F + E259G + N279S + K280Y + G303R + P321H + Q36OY + E390A + G476A | |
| H1* + G7A + D118T + W140Y + R180* + S181* + T182S + E189P + N194F + L205Y + | 1.1 |
| Y242F + E259G + M260W + N279S + K280Y + S302Y + E390A + G475K | |
| H1* + G7A + D118T + W140Y + R180* + S181* + T182S + E189P + N194F + L205Y + | 1.1 |
| Y242F + E259G + N279S + K280Y + G303R + E390A + G476A | |
| H1* + N3G + G7A + D118T + W140Y + R180* + S181* + T182S + E189P + N194F + | 1.1 |
| L205Y + Y242F + E259G + N279S + K280Y + S302T + E390A + G475K | |
| H1* + N3D + G7A + D118T + S132D + W140Y + R180* + S181* + T182S + E189P + | 1.2 |
| N194F + L205Y + Y242F + E259G + N279S + K280Y + G303R + E390A + H420A + | |
| G475K | |
| H1* + G7A + R37N + D118T + W140Y + R180* + S181* + T182S + E189P + N194F + | 1.2 |
| L205Y + Y242F + E259G + M260D + N279S + K280Y + G303R + E390A + G475K | |
| H1* + G7A + D118T + W140Y + R180* + S181* + T182S + E189P + N194F + L205Y + | 1.1 |
| Y242F + E259G + M260D + N279S + K280Y + G303R + E390A + G475K | |
| H1* + G7A + R37N + D118T + W140Y + R180* + S181* + T182S + E189P + N194F + | 1.1 |
| L205Y + Y242F + E259G + M260D + N279S + K280Y + G303R + E390A + G475K | |
| H1* + N3D + G7A + D118T + W140Y + R180* + S181* + T182S + E189P + N194F + | 1.1 |
| L205Y + Y242F + E259G + N279S + K280Y + G303R + E390A + H420A + G475K | |
| H1* + G7A + D118T + W140Y + R180* + S181* + T182S + E189P + N194F + L205Y + | 1.1 |
| Y242F + E259G + M260D + N279S + K280Y + G303R + N394D + G475K | |
| H1* + G7A + R37A + D118T + W140Y + R180* + S181* + T182S + E189P + N194F + | 1.1 |
| L205Y + Y242F + E259G + M260W + N279S + K280Y + G303R + E390A + N470M + | |
| G475K | |
| H1* + G7A + D118T + S132L + W140Y + R180* + S181* + T182S + E189P + N194F + | 1.1 |
| L205Y + Y242F + E259G + M260D + N279S + K280Y + G303R + E390A + G475K | |
| H1* + G7A + D118T + W140Y + R180* + S181* + T182S + E189P + N194F + L205Y + | 1.1 |
| Y242F + E259G + N279S + K280Y + S302T + E390A + G475K | |
| H1* + G7A + D118T + W140Y + R180* + S181* + T182S + E189P + N194F + L205Y + | 1.1 |
| Y242F + E259G + N279S + K280Y + G303R + N394D + H420A + G475K | |
| H1* + N3D + G7A + D118T + W140Y + R180* + S181* + T182S + E189P + N194F + | 1.1 |
| L205Y + Y242F + E259G + N279S + K280Y + S302T + E390A + G475K | |

The dimensions and values disclosed herein are not to be understood as being strictly limited to the exact numerical values recited. Instead, unless otherwise specified, each such dimension is intended to mean both the recited value and a functionally equivalent range surrounding that value.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. An alpha-amylase variant of a parent alpha-amylase, wherein said variant comprises a modification at one or more position corresponding to positions: 303, 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering, and wherein the said variant has 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the SEQ ID NO: 1, 2, 3, 4, 5, 6, 7, 8, 9, 10, 11, 12, 13 or 14 and wherein said variant has an improved performance, such as an improved wash performance for a measure of specific activity of Improvement Factor (IF)>1.0, when compared to said parent polypeptide, preferably SEQ ID NO: 2 or 3.

2. The variant according to claim 1, wherein said modification in at least one position corresponding to positions: G303, H1, H2, N3, G7, R26, A31, R37, T40, A41, N54, V56, A60, L63, K72, G73, K93, N94, Q98, V103, V104, M105, N106, A109, G110, A111, G113, T114, F116, V117, D118, D123, N126, N128, T131, S132, T134, Y135, Q136, I137, Q138, W140, K142, D144, P146, T151, Y152, F155, W159, Y160, T165, W167, E169, K172, L173, N174, I176, Y177, K178, A185, W188, E189, E193, N194, N196, F202, A203, L205, T224, N225, Y242, S243, F244, V252, T256, K258, E259, M260, F261, F266, N276, N279, K280, W283, S286, S302, T315, H320, P321, M322, H323, W346, L354, E359, Q360, G361, Y362, V365, Y371, A383, E390, N394, F395, D405, 1409, R414, H420, N422, T427, G455, K457, P458, N470, S472, V473, N474, G475 and G476, using SEQ ID NO: 1 for numbering and wherein the said variant has 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the SEQ ID NO. 1, 2, 3, 4, 5, 6, 7, 8, 9, 10, 11, 12, 13 or 14.

3. The variant according to claim 2, wherein said modification in at least one position corresponding to positions: G303R, H1*, H2*, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, R180*, R180Q, R180S, S181*, S181L, S181T, T182*, T182D, T182E, T182G, T182I, T182L, T182M, T182S, G183*, G183P, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering and wherein the said variant has 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the SEQ ID NO. 1, 2, 3, 4, 5, 6, 7, 8, 9, 10, 11, 12, 13 or 14.

4. The variant according to claim 1, wherein said variant comprises a deletion at two or more positions selected from the group consisting of: R180*+S181*; R180*+T182*; R180*+G183*; S181*+T182*; S181*+G183*; and T182*+G183*; using SEQ ID NO: 1 for numbering.

5. The variant according to claim 1, wherein the variant comprises or consists of:
(a) a substitution and/or deletion of two, three or four positions in the parent alpha-amylase said positions corresponding to position 180, 181, 182, and 183 using SEQ ID NO: 1 for numbering; and
(b) a modification at one or more positions corresponding to positions: G303R, H1*, H2, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering, and wherein the said variant has 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the SEQ ID NO: 1, 2, 3, 4, 5, 6, 7, 8, 9, 10, 11, 12, 13 or 14.

6. The variant according to claim 1, wherein the variant comprises of one or more modifications selected from the group consisting of:
H1*+G7A+R180*+S181*, H1*+G7A+R180*+S181*+Y242F, H1*+G7A+R180*+S181*+Y242F+N279S, H1*+G7A+R180*+S181*+Y242F+N279S+G303R, H1*+G7A+R180*+S181*+Y242F+E259G+N279S+G303R, H1*+G7A+R180*+S181*+Y242F+E259G+N279S+G303R+E390A, H1*+G7A+R180*+S181*+Y242F+E259G+N279S+G303R+E390A+G475K, H1*+G7A+R180*+S181*+Y242F+E259G+N279S+E390A+G476A, H1*+G7A+R180*+S181*+Y242F+E259G+N279S+G303R+E390A+G476A, H1*+G7A+R180*+S181*+D118T+Y242F+E259G+N279S+G303R+E390A+G475K, H1*+G7A+R180*+S181*+D118T+W140Y+Y242F+E259G+N279S+E390A+G476A, H1*+G7A+R180*+S181*+D118T+W140Y+Y242F+E259G+N279S+G303R+E390A+G476A, H1*+G7A+R180*+S181*+D118T+W140Y+Y242F+E259G+N279S+G303R+E390A+G475K, H1*+G7A+R180*+S181*+D118T+W140Y+Y242F+E259G+N279S+E390A+G476A, H1*+G7A+R180*+S181*+D118T+W140Y+Y242F+E259G+N279S+G303R+E390A+G476A, H1*+G7A+R180*+S181*+D118T+W140Y+N194F+Y242F+E259G+N279S+G303R+E390A+G475K, H1*+G7A+R180*+S181*+D118T+W140Y+N194F+Y242F+E259G+N279S+E390A+G476A, H1*+G7A+R180*+S181*+D118T+W140Y+N194F+Y242F+E259G+N279S+G303R+E390A+G476A, H1*+G7A+R180*+S181*+D118T+W140Y+N194F+Y242F+E259G+K280Y+N279S+G303R+E390A+G475K, H1*+G7A+R180*+S181*+D118T+W140Y+N194F+Y242F+E259G+K280Y+N279S+E390A+G476A, H1*+G7A+R180*+S181*+D118T+W140Y+N194F+Y242F+E259G+K280Y+N279S+G303R+E390A+G476A, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

7. The variant according to claim 1, wherein the variant comprises or consists of one or more modifications selected from the group consisting of:
H1*+G7A+R180*+S181*+Y242F+E259G+N279S+G303R+E390A+G475K, H1*+G7A+S181*+T182*+Y242F+E259G+N279S+G303R+E390A+G475K, H1*+G7A+R180*+S181*+T182S+Y242F+E259G+N279S+G303R+E390A+G475K, H1*+G7A+R180*+T182L+G183*+Y242F+E259G+N279S+G303R+E390A+G475K, H1*+G7A+S181 L+T182*+G183*+Y242F+E259G+N279S+G303R+E390A+G475K, H1*+G7A+R180S+T182*+G183*+Y242F+E259G+N279S+G303R+E390A+G475K, H1*+G7A+A60P+R180*+S181*+Y242F+E259G+N279S+G303R+E390A+G475K, H1*+G7A+M105L+R180*+S181*+Y242F+E259G+N279S+G303R+E390A+G475K, H1*+G7A+M105I+R180*+S181*+Y242F+E259G+N279S+G303R+E390A+G475K, H1*+G7A+R180*+S181*+T182D+Y242F+E259G+N279S+G303R+E390A+G475K, H1*+G7A+R180*+S181*+T182E+Y242F+E259G+N279S+G303R+E390A+G475K, H1*+G7A+R180*+S181*+T182G+Y242F+E259G+N279S+G303R+E390A+G475K, H1*+G7A+R180*+S181*+T182M+Y242F+E259G+N279S+G303R+E390A+G475K, H1*+G7A+R180*+S181*+A185K+Y242F+E259G+N279S+G303R+E390A+G475K, H1*+G7A+R180*+S181*+A185D+Y242F+E259G+N279S+G303R+E390A+G475K, H1*+G7A+R180*+S181*+A185Q+Y242F+E259G+N279S+G303R+E390A+G475K, H1*+G7A+R180*+S181*+A185N+Y242F+E259G+N279S+G303R+E390A+G475K, H1*+G7A+R180*+S181*+A185R+Y242F+E259G+N279S+G303R+E390A+G475K, H1*+G7A+R180*+S181*+A185E+Y242F+E259G+N279S+G303R+E390A+G475K, H1*+G7A+R180*+S181*+E189P+Y242F+E259G+N279S+G303R+E390A+G475K, H1*+G7A+R180*+S181*+L205Y+Y242F+E259G+N279S+G303R+E390A+G475K, H1*+G7A+R180*+S181*+L205F+Y242F+E259G+N279S+G303R+E390A+G475K, H1*+G7A+R180*+S181*+Y242F+S243Q+E259G+N279S+G303R+E390A+G475K, H1*+G7A+R180*+S181*+Y242F+F244L+E259G+N279S+G303R+E390A+G475K, H1*+G7A+R180*+S181*+Y242F+F244I+E259G+N279S+G303R+E390A+G475K, H1*+G7A+R180*+S181*+Y242F+E259G+F266Y+N279S+G303R+E390A+G475K, H1*+G7A+R180*+S181*+N194F+Y242F+E259G+N279S+G303R+E390A+G475K, H1*+G7A+R180*+S181*+N196F+Y242F+E259G+N279S+G303R+E390A+G475K, H1*+G7A+R180*+S181*+N194F+L205Y+Y242F+E259G+N279S+G303R+E390A+G475K, H1*+G7A+R180*+S181*+N194F+L205F+Y242F+E259G+N279S+G303R+E390A+G475K, H1*+G7A+R180*+S181*+N196F+L205Y+Y242F+E259G+N279S+G303R+E390A+G475K, H1*+G7A+R180*+S181*+E189P+Y242F+E259G+N276F+N279S+G303R+E390A+G475K, H1*+G7A+R180*+S181*+E189P+Y242F+E259G+N276Y+N279S+G303R+E390A+G475K, H1*+G7A+R180*+S181*+E189P+Y242F+E259G+N276W+N279S+G303R+E390A+G475K, H1*+G7A+R180*+S181*+E189P+Y242F+E259G+N279S+K280Y+G303R+E390A+G475K, H1*+G7A+R180*+S181*+E189P+Y242F+E259G+N279S+K280W+G303R+E390A+G475K, H1*+G7A+R180*+S181*+E189P+L205F+Y242F+E259G+N276F+N279S+G303R+E390A+G475K, H1*+G7A+R180*+S181*+E189P+L205F+Y242F+E259G+N276Y+N279S+G303R+E390A+G475K, H1*+G7A+R180*+S181*+E189P+L205F+Y242F+E259G+N276W+N279S+G303R+E390A+G475K, H1*+G7A+R180*+S181*+E189P+L205F+Y242F+E259G+N279S+K280F+G303R+E390A+G475K, H1*+G7A+R180*+S181*+E189P+L205F+Y242F+E259G+N279S+K280Y+G303R+E390A+G475K, H1*+G7A+R180*+S181*+E189P+L205F+Y242F+E259G+N279S+K280W+G303R+E390A+G475K, H1*+G7A+R180*+S181*+E189P+L205Y+Y242F+E259G+N276F+N279S+G303R+E390A+G475K, H1*+G7A+R180*+S181*+E189P+L205Y+Y242F+E259G+N276Y+N279S+G303R+E390A+G475K, H1*+G7A+R180*+S181*+E189P+L205Y+Y242F+E259G+N276W+N279S+G303R+E390A+G475K, H1*+G7A+R180*+S181*+E189P+L205Y+Y242F+E259G+N279S+K280F+G303R+E390A+G475K, H1*+G7A+R180*+S181*+E189P+L205Y+Y242F+E259G+N279S+K280Y+G303R+E390A+G475K, H1*+G7A+R180*+S181*+E189P+L205Y+Y242F+E259G+N279S+K280W+G303R+E390A+G475K, H1*+G7A+G113S+F116W+D123N+S181*+T182*+Y242F+E259G+N279S+G303R+E390A+G475K, H1*+G7A+F116L+D123N+N126D+T131 I+S181*+T182*+F202Y+Y242F+E259G+N279S+G303R+E390A+G475K, H1*+G7S+T40N+Q98L+R180*+S181*+Y242F+E259G+N279S+G303R+Y362V+E390A+G475K H1*+G7A+R180*+S181*+Y242F+E259G+N279S+G303R+M322G+E359W+Q360W+E390A+G475K, H1*+G7A+R180*+S181*+Y242F+E259G+N279S+G303R+M322G+Q360W+E390A+G475K, H1*+G7A+R180*+S181*+Y242F+E259G+N279S+G303R+E359W+Q360W+E390A+G475K, H1*+G7S+R180*+S181*+Y242F+E259G+F261S+N279S+G303R+Y362V+E390A+G475K, H1*+G7A+T40N+Q98L+R180*+S181*+Y242F+E259G+N279S+G303R+E390A+G475K, H1*+G7S+T40N+Q98L+R180*+S181*+Y242F+E259G+N279S+G303R+E390A+G475K, H1*+G7A+R180*+S181*+Y242F+E259G+F261S+N279S+S286K+G303R+Y362V+E390A+G475K, H1*+G7A+R180*+S181*+Y242F+E259G+N279S+W283N+S286K+G303R+H320T+H323A+E390A+G475K, H1*+G7A+R180*+S181*+Y242F+E259G+N279S+G303R+P321H+M322Y+Q360Y+E390A+G475K, H1*+G7A+D123N+I176V+R180*+S181*+Y242F+E259G+N279S+G303R+E390A+G475K, H1*+G7A+G113A+F116W+D123N+T131V+Q136K+E169Q+K172Q+R180*+S181*+Y242F+E259G+N279S+G303R+E390A+G475K, H1*+G7A+G113S+F116W+D123N+R180*+S181*+Y242F+E259G+N279S+G303R+E390A+G475K, H1*+G7A+F116L+D123N+N126D+T131 I+R180*+S181*+F202Y+Y242F+E259G+N279S+G303R+E390A+G475K, H1*+G7A+N106D+A109G+F116W+D123N+N126D+T131I+T165V+N174S+R180*+S181*+T182I+F202Y+Y242F+E259G+N279S+G303R+E390A+G475K, H1*+G7S+T40N+Q98L+R180*+S181*+Y242F+E259G+N279S+G303R+H320T+E390A+G475K H1*+G7S+T40N+Q98L+R180*+S181*+Y242F+E259G+N279S+G303R+Y362V+E390A+G475K H1*+G7A+R180*+S181*+Y242F+E259G+N279S+G303R+M322G+E390A+G475K, H1*+G7A+R180*+S181*+Y242F+E259G+N279S+G303R+M322G+Q360W+E390A+G475K, H1*+G7A+T40N+R180*+S181*+Y242F+E259G+N279S+G303R+E390A+G475K, H1*+G7S+Q98L+R180*+S181*+Y242F+E259G+N279S+G303R+E390A+G475K, H1*+G7S+R180*+S181*+Y242F+E259G+F261K+N279S+G303R+E390A+G475K, H1*+G7S+R180*+S181*+Y242F+E259G+N279S+G303R+Y362V+E390A+G475K, H1*+G7A+T40N+Q98L+R180*+S181*+Y242F+E259G+N279S+G303R+E390A+G475K, H1*+G7A+R180*+S181*+Y242F+E259G+N279S+G303R+Y362V+E390A+G475K, H1*+G7S+R180*+S181*+Y242F+E259G+F261S+N279S+G303R+E390A+G475K, H1*+G7A+1137F+R180*+S181*+Y242F+E259G+F261S+N279S+G303R+Y362V+E390A+G475K, H1*+G7A+R180*+S181*+Y242F+E259G+N279S+S286K+G303R+H323A+E390A+G475K, H1*+G7A+R180*+S181*+Y242F+E259G+N279S+G303R+H320T+H323A+E390A+G475K, H1*+G7A+R180*+S181*+Y242F+E259G+N279S+G303R+P321H+M322Y+E390A+G475K, H1*+G7A+R180*+S181*+Y242F+E259G+N279S+G303R+P321H+Q360Y+E390A+G475K, H1*+G7A+R180*+S181*+Y242F+E259G+N279S+G303R+M322Y+Q360Y+E390A+G475K, H1*+G7S+T40N+R180*+S181*+Y242F+E259G+N279S+G303R+Y362V+E390A+G475K, H1*+G7S+R180*+S181*+Y242F+E259G+F261S+N279S+G303R+Y362V+E390A+G475K, H1*+G7A+R180*+S181*+Y242F+E259G+F261S+N279S+S286K+G303R+Y362V+E390A+G475K, H1*+G7A+R180*+S181*+Y242F+E259G+N279S+S286L+G303R+E390A+G475K, H1*+G7A+G110K+R180*+S181*+Y242F+E259G+N279S+G303R+E390A+G475K, H1*+G7A+W140S+R180*+S181*+Y242F+E259G+N279S+G303R+E390A+G475K, H1*+G7A+W167P+R180*+S181*+Y242F+E259G+N279S+G303R+E390A+G475K, H1*+G7A+R180*+S181*+Y242F+E259G+N279S+G303R+H323A+E390A+G475K, H1*+G7A+R180*+S181*+Y242F+E259G+N279S+W283N+G303R+H320T+H323A+E390A+G475K, H1*+G7A+R180*+S181*+Y242F+E259G+N279S+G303R+H320T+E390A+G475K, H1*+G7A+R180*+S181*+Y242F+E259G+N279S+G303R+P321H+E390A+G475K, H1*+G7A+R180*+S181*+Y242F+E259G+N279S+G303R+M322Y+E390A+G475K, H1*+G7A+R180*+S181*+Y242F+E259G+N279S+G303R+Q360Y+E390A+G475K, H1*+G7S+T40N+Q98L+R180*+S181*+Y242F+E259G+N279S+G303R+E390A+G475K, H1*+G7A+Q98L+R180*+S181*+Y242F+E259G+F261K+N279S+G303R+Y362V+E390A+G475K, H1*+G7A+Q98L+R180*+S181*+Y242F+E259G+N279S+G303R+E390A+G475K, H1*+G7A+R180*+S181*+Y242F+E259G+F261S+N279S+G303R+Y362V+E390A+G475K, H1*+G7A+R180*+S181*+Y242F+E259G+N279S+S286L+G303R+H320T+E390A+G475K, H1*+G7A+R180*+S181*+Y242F+E259G+F261S+N279S+G303R+E390A+G475K, H1*+G7A+W140Y+R180*+S181*+T182D+N194F+L205Y+Y242F+E259G+N279S+G303R+E390A+G475K, H1*+G7A+W140Y+R180*+S181*+T182D+N194F+L205V+Y242F+E259G+N279S+G303R+E390A+G475K, H1*+G7A+D118T+W140Y+R180*+S181*+T182D+N194F+L205Y+Y242F+E259G+N279S+G303R+E390A+G475K, H1*+G7A+F116N+W140Y+R180*+S181*+T182D+N194F+L205Y+Y242F+E259G+N279S+G303R+E390A+G475K, H1*+G7A+W140Y+R180*+S181*+E189P+N194F+L205Y+Y242F+E259G+N279S+K280Y+G303R+E390A+G475K, H1*+G7A+D118T+W140Y+R180*+S181*+E189P+N194F+L205Y+Y242F+E259G+N279S+K280Y+G303R+E390A+G475K, H1*+G7A+F116N+W140Y+R180*+S181*+E189P+N194F+L205Y+Y242F+E259G+N279S+K280Y+G303R+E390A+G475K, H1*+G7A+W140Y+R180*+S181*+T182D+E189P+N194F+L205Y+Y242F+E259G+N279S+G303R+E390A+G475K, H1*+G7A+T134D+W140Y+R180*+S181*+E189P+N194F+L205V+Y242F+E259G+N279S+G303R+E390A+G475K, H1*+G7A+F116N+W140Y+R180*+S181*+T182D+E189P+N194F+L205Y+Y242F+E259G+N279S+G303R+E390A+G475K, H1*+G7A+W140Y+R180*+S181*+T182S+N194F+L205Y+Y242F+E259G+N279S+G303R+E390A+G475K, H1*+G7A+W140Y+R180*+S181*+T182S+N194F+L205V+Y242F+E259G+N279S+G303R+E390A+G475K, H1*+G7A+D118T+W140Y+R180*+S181*+T182S+N194F+L205Y+Y242F+E259G+N279S+G303R+E390A+G475K, H1*+G7A+F116N+W140Y+R180*+S181*+T182S+N194F+L205Y+Y242F+E259G+N279S+G303R+E390A+G475K, H1*+G7A+W140Y+R180*+S181*+T182S+E189P+N194F+L205Y+Y242F+E259G+N279S+G303R+E390A+G475K, H1*+G7A+W140Y+R180*+S181*+T182S+E189P+N194F+L205V+Y242F+E259G+N279S+G303R+E390A+G475K, H1*+G7A+F116N+W140Y+R180*+S181*+T182S+E189P+N194F+L205Y+Y242F+E259G+N279S+K280Y+G303R+E390A+G475K, H1*+G7A+D123N+W140Y+R180*+S181*+N194F+L205V+Y242F+E259G+N279S+G303R+E390A+G475K, H1*+G7A+D123N+W140Y+I176V+R180*+S181*+N194F+L205V+Y242F+E259G+N279S+G303R+E390A+G475K, H1*+G7A+G113S+F116W+D123N+W140Y+R180*+S181*+N194F+L205V+Y242F+E259G+N279S+G303R+E390A+G475K, H1*+G7A+F116L+D123N+N126D+T131I+W140Y+R180*+S181*+N194F+F202Y+L205V+Y242F+E259G+N279S+G303R+E390A+G475K, H1*+G7A+N106D+A109G+W140Y+R180*+S181*+N194F+F202Y+L205V+Y242F+E259G+N279S+G303R+E390A+G475K, H1*+G7A+D123N+W140Y+R180*+S181*+N194F+L205Y+Y242F+E259G+N279S+G303R+E390A+G475K, H1*+G7A+D123N+W140Y+I176V+R180*+S181*+N194F+L205Y+Y242F+E259G+N279S+G303R+E390A+G475K, H1*+G7A+G113S+F116W+D123N+W140Y+R180*+S181*+N194F+L205Y+Y242F+E259G+N279S+G303R+E390A+G475K, H1*+G7A+F116L+D123N+N126D+T131I+W140Y+R180*+S181*+N194F+F202Y+L205Y+Y242F+E259G+N279S+G303R+E390A+G475K, H1*+G7A+Q98L+G110K+P146S+R180*+S181*+Y242F+E259G+N279S+G303R+A383D+E390A+G475K, H1*+N3S+G7A+Q98L+G110K+R180*+S181*+Y242F+V252+E259G+N279S+G303R+E390A+G475K, H1*+G7H+Q98L+R180*+S181*+Y242F+E259G+N279S+G303R+A383D+E390A+D405G+G475K, H1*+G7H+Q98L+G110K+R180*+S181*+Y242F+E259G+N279S+G303R+E390A+G475K, H1*+G7A+Q98L+G110K+S132D+Y135E+R180*+S181*+Y242F+E259G+N279S+G303R+T315A+E390A+G475K, H1*+G7H+G110K+R180*+S181*+Y242F+E259G+N279S+G303R+E390A+G475K, H1*+A60V+Q98L+W140Y+R180*+S181*+N194F+L205Y+Y242F+E259G+N279S+G303R+A383D+E390A+G475K, H1*+G7A+W140Y+T151A+R180*+S181*+N194F+L205Y+Y242F+K258V+E259V+N279S+G303R+E390A+G475K, H1*+N3S+G7A+N94D+Q98L+W140Y+R180*+S181*+N194F+L205Y+Y242F+V252+E259G+N279S+G303R+E390A+G475K, H1*+G7A+Q98L+G110K+R180*+S181*+Y242F+T256A+E259G+N279S+G303R+E390A+G475K, H1*+G7A+Q98L+G110K+R180*+S181*+Y242F+E259G+N279S+G303R+A383D+E390A+G475K, H1*+N3S+G7A+N94D+Q98L+G110K+R180*+S181*+Y242F+V252I+E259G+N279S+G303R+E390A+G475K, H1*+G7A+Q98L+G110K+R180*+S181*+Y242F+E259G+N279S+G303R+E390A+G475K, H1*+G7H+Q98L+G110K+R180*+S181*+Y242F+E259G+N279S+G303R+E390A+G475K, H1*+G7A+W140Y+T151A+R180*+S181*+N194F+L205Y+Y242F+K258V+E259V+N279S+G303R+E390A+G475K, H1*+G7A+Q98L+G110K+R180*+S181*+Y242F+E259G+N279S+G303R+E390A+G475K, H1*+G7A+R180*+S181*+Y242F+E259G+N279S+S286L+G303R+Y362V+E390A+G475K, H1*+G7A+R180*+S181*+Y242F+E259G+N279S+W283N+S286K+G303R+E390A+G475K, H1*+G7A+R180*+S181*+Y242F+E259G+N279S+W283N+G303R+H320T+E390A+G475K, H1*+G7A+R180*+S181*+Y242F+E259G+N279S+W283N+G303R+H323A+E390A+G475K, H1*+G7A+R180*+S181*+Y242F+E259G+N279S+S286K+G303R+H320T+E390A+G475K, H1*+G7A+R180*+S181*+Y242F+E259G+N279S+S286K+G303R+Y362V+E390A+G475K, H1*+G7S+R180*+S181*+Y242F+E259G+N279S+G303R+E390A+G475K, H1*+G7A+Q98L+R180*+S181*+Y242F+E259G+N279S+G303R+E390A+G475K, H1*+G7A+R180*+S181*+Y242F+E259G+F261K+N279S+G303R+E390A+G475K, H1*+G7A+R180*+S181*+Y242F+E259G+N279S+G303R+M322G+E359W+E390A+G475K, H1*+G7A+F116N+W140Y+R180*+S181*+T182D+E189P+N194F+L205Y+Y242F+E259G+N279S+K280Y+G303R+E390A+G475K, H1*+G7A+F116N+W140Y+R180*+S181*+T182D+E189P+N194F+Y242F+E259G+N279S+K280Y+G303R+E390A+G475K, H1*+G7A+F116N+W140Y+R180*+S181*+T182S+E189P+N194F+Y242F+E259G+N279S+K280Y+G303R+E390A+G475K, H1*+G7A+F116N+W140Y+R180*+S181*+T182D+N194F+L205Y+Y242F+E259G+F266Y+N279S+G303R+E390A+G475K, H1*+G7A+F116N+W140Y+R180*+S181*+A185Q+N194F+L205Y+Y242F+E259G+F266Y+N279S+G303R+E390A+G475K, H1*+G7A+F116N+W140Y+R180*+S181*+T182D+A185Q+N194F+L205Y+Y242F+E259G+F266Y+N279S+G303R+E390A+G475K, H1*+G7A+F116N+W140Y+R180*+S181*+N194F+L205Y+Y242F+E259G+F266Y+N279S+G303R+E390A+G475K, H1*+G7A+F116N+W140Y+R180*+S181*+A185Q+N194F+L205Y+Y242F+E259G+N279S+G303R+E390A+G475K, H1*+G7A+F116N+W140Y+R180*+S181*+T182D+A185Q+N194F+L205Y+Y242F+E259G+N279S+G303R+E390A+G475K, H1*+G7A+D118T+W140Y+R180*+S181*+E189P+N194F+Y242F+E259G+N279S+K280Y+G303R+E390A+G475K, H1*+G7A+D118T+W140Y+R180*+S181*+T182S+E189P+N194F+L205Y+Y242F+E259G+N279S+K280Y+G303R+E390A+G475K, H1*+G7A+D118T+W140Y+R180*+S181*+T182D+N194F+L205Y+Y242F+E259G+F266Y+N279S+G303R+E390A+G475K, H1*+G7A+D118T+W140Y+R180*+S181*+A185Q+N194F+L205Y+Y242F+E259G+F266Y+N279S+G303R+E390A+G475K, H1*+G7A+D118T+W140Y+R180*+S181*+T182D+A185Q+N194F+L205Y+Y242F+E259G+F266Y+N279S+G303R+E390A+G475K, H1*+G7A+D118T+W140Y+R180*+S181*+N194F+L205Y+Y242F+E259G+F266Y+N279S+G303R+E390A+G475K, H1*+G7A+D118T+W140Y+R180*+S181*+A185Q+N194F+L205Y+Y242F+E259G+N279S+G303R+E390A+G475K, H1*+G7A+D118T+W140Y+R180*+S181*+T182D+A185Q+N194F+L205Y+Y242F+E259G+N279S+G303R+E390A+G475K, H1*+G7A+W140Y+R180*+S181*+E189P+N194F+L205V+Y242F+E259G+N279S+K280Y+G303R+E390A+G475K, H1*+G7A+W140Y+R180*+S181*+T182D+E189P+N194F+L205V+Y242F+E259G+N279S+K280Y+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+T182S+E189P+N194F+Y242F+E259G+N279S+K280Y+G303R+E390A+G475K, H1*+G7A+W140Y+R180*+S181*+T182D+N194F+L205V+Y242F+E259G+F266Y+N279S+G303R+E390A+G475K, H1*+G7A+W140Y+R180*+S181*+A185Q+N194F+L205V+Y242F+E259G+F266Y+N279S+G303R+E390A+G475K, H1*+G7A+W140Y+R180*+S181*+T182D+A185Q+N194F+L205V+Y242F+E259G+F266Y+N279S+G303R+E390A+G475K, H1*+G7A+W140Y+R180*+S181*+N194F+L205V+Y242F+E259G+F266Y+N279S+G303R+E390A+G475K, H1*+G7A+W140Y+R180*+S181*+A185Q+N194F+L205V+Y242F+E259G+N279S+G303R+E390A+G475K, H1*+G7A+W140Y+R180*+S181*+T182D+A185Q+N194F+L205V+Y242F+E259G+N279S+G303R+E390A+G475K, H1*+G7A+W140Y+R180*+S181*+T182D+E189P+N194F+L205Y+Y242F+E259G+N279S+K280Y+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+T182S+E189P+N194F+L205Y+Y242F+E259G+N279S+K280Y+G303R+E390A+G475K, H1*+G7A+W140Y+R180*+S181*+T182S+E189P+N194F+Y242F+E259G+N279S+K280Y+G303R+E390A+G475K, H1*+G7A+W140Y+R180*+S181*+T182D+N194F+L205Y+Y242F+E259G+F266Y+N279S+G303R+E390A+G475K, H1*+G7A+W140Y+R180*+S181*+A185Q+N194F+L205Y+Y242F+E259G+F266Y+N279S+G303R+E390A+G475K, H1*+G7A+W140Y+R180*+S181*+T182D+A185Q+N194F+L205Y+Y242F+E259G+F266Y+N279S+G303R+E390A+G475K, H1*+G7A+W140Y+R180*+S181*+A185Q+N194F+L205Y+Y242F+E259G+N279S+G303R+E390A+G475K, H1*+G7A+W140Y+R180*+S181*+T182D+A185Q+N194F+L205Y+Y242F+E259G+N279S+G303R+E390A+G475K, H1*+G7A+F116N+W140Y+R180*+S181*+E189P+N194F+Y242F+E259G+N279S+K280Y+G303R+E390A+G475K, H1*+G7A+D118T+W140Y+R180*+S181*+T182D+E189P+N194F+L205Y+Y242F+E259G+N279S+K280Y+G303R+E390A+G475K, H1*+G7A+D118T+W140Y+R180*+S181*+T182D+E189P+N194F+Y242F+E259G+N279S+K280Y+G303R+E390A+G475K, H1*+G7A+D118T+W140Y+R180*+S181*+T182S+E189P+N194F+Y242F+E259G+N279S+K280Y+G303R+E390A+G475K, H1*+G7A+W140Y+R180*+S181*+E189P+N194F+Y242F+E259G+N279S+K280Y+G303R+E390A+G475K, H1*+G7A+W140Y+R180*+S181*+E189P+N194F+Y242F+E259G+N279S+K280Y+G303R+E390A+G475K, H1*+G7A+W140Y+R180*+S181*+N194F+L205Y+Y242F+E259G+F266Y+N279S+G303R+E390A+G475K, H1*+G7A+W140Y+R180*+S181*+N194F+L205Y+Y242F+E259G+N279S+S302T+G303R+E390A+G475K, H1*+G7A+W140Y+R180*+S181*+N194F+L205Y+Y242F+E259G+N279S+G303R+E390A+N474F+G475K, H1*+G7A+W140Y+R180*+S181*+N194F+L205Y+Y242F+E259G+N279S+G303R+E390A+N422V+G475K, H1*+G7A+W140Y+R180*+S181*+N194F+L205Y+Y242F+E259G+N279S+G303R+E390A+G476A, H1*+G7A+W140Y+R180*+S181*+N194F+L205Y+Y242F+E259G+N279S+G303R+N394W+G475K, H1*+G7A+W140Y+R180*+S181*+N194F+L205Y+Y242F+E259G+N279S+G303R+E390A+N474L, H1*+G7A+W140Y+R180*+S181*+N194F+L205Y+Y242F+E259G+N279S+S302T+E390A+G475K, H1*+G7A+W140Y+R180*+S181*+N194F+L205Y+Y242F+E259G+N279S+G303R+E390A+G475E, H1*+G7A+W140Y+R180*+S181*+N194F+L205Y+Y242F+E259G+F261P+N279S+G303R+E390A+G475K, H1*+G7A+W140Y+R180*+S181*+N194F+L205Y+Y242F+E259G+N279S+G303R+E390A+N474F, H1*+G7A+W140Y+R180*+S181*+N194F+L205Y+Y242F+E259G+N279S+S302Y+E390A+G475K, H1*+G7A+W140Y+R180*+S181*+N194F+L205Y+Y242F+E259G+N279S+G303R+E390A+N470A+G475K, H1*+G7A+W140Y+R180*+S181*+N194F+L205Y+Y242F+T256D+N279S+G303R+E390A+G475K, H1*+G7A+W140Y+R180*+S181*+N194F+L205Y+Y242L+E259G+N279S+G303R+W346N+E390A+G475K, H1*+G7A+W140Y+R180*+S181*+N194F+L205Y+Y242F+E259G+N279S+G303R+E390A+H420P+G475K, H1*+G7A+R26Y+W140Y+R180*+S181*+N194F+L205Y+Y242F+E259G+N279S+G303R+E390A+G475K, H1*+G7A+R37A+W140Y+R180*+S181*+N194F+L205Y+Y242F+E259G+N279S+G303R+G475K, H1*+W140Y+R180*+S181*+N194F+L205Y+Y242F+E259G+N279S+G303R+Q360Y+E390A+G475K, H1*+G7A+R37N+W140Y+R180*+S181*+N194F+L205Y+Y242F+E259G+N279S+G303R+G475K, H1*+W140Y+R180*+S181*+N194F+L205Y+Y242F+E259G+N279S+G303R+E390A+R414M+G475K, H1*+W140Y+R180*+S181*+N194F+L205Y+Y242F+E259G+F261P+N279S+G303R+E390A+G475K, H1*+W140Y+R180*+S181*+N194F+L205Y+Y242F+E259G+N279S+G303R+H323W+E390A+G475K, H1*+W140Y+R180*+S181*+N194F+L205Y+Y242F+E259G+N279S+G303R+P321Y+E390A+G475K, H1*+W140Y+R180*+S181*+N194F+L205Y+Y242F+E259G+N279S+G303R+Q360M+E390A+G475K, H1*+G7A+W140Y+R180*+S181*+N194F+L205Y+Y242F+E259G+N279S+G303R+Y371S+E390A+G475K, H1*+G7A+W140Y+R180*+S181*+T182D+A185Q+N194F+Y242F+S243Q+E259G+N279S+K280Y+G303R+E390A+G475K, H1*+G7A+W140Y+R180*+S181*+T182D+Y242F+S243Q+E259G+F266Y+N279S+K280Y+G303R+E390A+G475K, H1*+G7A+W140Y+R180*+S181*+A185Q+N194F+Y242F+S243Q+E259G+F266Y+N279S+K280Y+G303R+E390A+G475K, H1*+G7A+W140Y+R180*+S181*+T182D+A185Q+Y242F+E259G+N279S+K280Y+G303R+E390A+G475K, H1*+G7A+W140Y+R180*+S181*+T182D+E189P+Y242F+S243Q+E259G+N279S+K280Y+G303R+E390A+G475K, H1*+G7A+W140Y+R180*+S181*+A185Q+Y242F+E259G+F266Y+N279S+K280Y+G303R+E390A+T427L+G475K, H1*+G7A+W140Y+R180*+S181*+A185Q+E189P+Y242F+E259G+N279S+K280Y+G303R+E390A+G475K, H1*+G7A+W140Y+R180*+S181*+T182D+A185Q+E189P+N194F+Y242F+S243Q+E259G+N279S+K280Y+G303R+E390A+G475K, H1*+G7A+W140Y+R180*+S181*+T182D+A185Q+E189P+N194F+L205Y+Y242F+S243Q+E259G+N279S+K280Y+G303R+E390A+G475K, H1*+G7A+W140Y+R180*+S181*+T182D+E189P+L205Y+Y242F+E259G+F266Y+N279S+K280Y+G303R+E390A+G475K, H1*+G7A+W140Y+R180*+S181*+A185Q+E189P+N194F+Y242F+E259G+F266Y+N279S+K280Y+G303R+E390A+G475K, H1*+G7A+W140Y+R180*+S181*+A185Q+E189P+N194F+Y242F+S243Q+E259G+F266Y+N279S+K280Y+G303R+E390A+G475K, H1*+G7A+W140Y+R180*+S181*+A185Q+E189P+N194F+L205Y+Y242F+S243Q+E259G+F266Y+N279S+K280Y+G303R+E390A+G475K, H1*+G7A+W140Y+R180*+S181*+T182D+A185Q+E189P+N194F+L205Y+Y242F+E259G+F266Y+N279S+K280Y+G303R+E390A+G475K, H1*+G7A+W140Y+R180*+S181*+T182D+A185Q+E189P+N194F+Y242F+E259G+F266Y+N279S+K280Y+G303R+E390A+G475K, H1*+G7A+W140Y+R180*+S181*+A185Q+Y242F+S243Q+E259G+F266Y+N279S+K280Y+G303R+E390A+G475K, H1*+G7A+W140Y+R180*+S181*+T182D+A185Q+Y242F+S243Q+E259G+F266Y+N279S+G303R+E390A+G475K, H1*+G7A+W140Y+R180*+S181*+Y242F+S243Q+E259G+N279S+K280Y+G303R+E390A+G475K, H1*+G7A+W140Y+R180*+S181*+T182D+E189P+N194F+Y242F+E259G+N279S+K280Y+G303R+E390A+G475K, H1*+G7A+W140Y+R180*+S181*+A185Q+N194F+Y242F+S243Q+E259G+N279S+K280Y+G303R+E390A+G475K, H1*+G7A+W140Y+R180*+S181*+T182D+N194F+Y242F+S243Q+E259G+N279S+K280Y+G303R+E390A+G475K, H1*+G7A+W140Y+R180*+S181*+A185Q+E189P+N194F+Y242F+E259G+N279S+K280Y+G303R+E390A+G475K, H1*+G7A+W140Y+R180*+S181*+E189P+Y242F+S243Q+E259G+N279S+K280Y+G303R+E390A+G475K, H1*+G7A+W140Y+R180*+S181*+T182D+Y242F+S243Q+E259G+N279S+K280Y+G303R+E390A+G475K, H1*+G7A+W140Y+R180*+S181*+E189P+L205Y+Y242F+S243Q+E259G+N279S+K280Y+G303R+E390A+G475K, H1*+G7A+W140Y+R180*+S181*+Y242F+E259G+F266Y+N279S+K280Y+G303R+E390A+G475K, H1*+G7A+W140Y+R180*+S181*+T182D+A185Q+Y242F+E259G+N279S+G303R+E390A+G475K, H1*+G7A+W140Y+R180*+S181*+T182D+A185Q+N194F+Y242F+S243Q+E259G+N279S+G303R+E390A+G475K, H1*+G7A+W140Y+R180*+S181*+T182D+N194F+Y242F+S243Q+E259G+F266Y+N279S+G303R+E390A+G475K, H1*+G7A+W140Y+R180*+S181*+T182D+E189P+N194F+Y242F+S243Q+E259G+N279S+K280Y+G303R+E390A+G475K, H1*+G7A+W140Y+R180*+S181*+E189P+N194F+Y242F+E259G+F266Y+N279S+K280Y+G303R+E390A+G475K, H1*+G7A+W140Y+R180*+S181*+A185Q+E189P+N194F+L205Y+Y242F+S243Q+E259G+N279S+K280Y+G303R+E390A+G475K, H1*+G7A+W140Y+R180*+S181*+T182D+A185Q+N194F+Y242F+E259G+F266Y+N279S+G303R+E390A+G475K, H1*+G7A+W140Y+R180*+S181*+A185Q+N194F+Y242F+E259G+F266Y+N279S+K280Y+G303R+E390A+G475K, H1*+G7A+W140Y+R180*+S181*+A185Q+E189P+L205Y+Y242F+E259G+N279S+K280Y+G303R+E390A+G475K, H1*+G7A+W140Y+R180*+S181*+Y242F+S243Q+E259G+F266Y+N279S+K280Y+G303R+E390A+G475K, H1*+G7A+W140Y+R180*+S181*+T182D+E189P+L205Y+Y242F+S243Q+E259G+N279S+K280Y+G303R+E390A+I409L+G475K, H1*+G7A+A41D+W140Y+R180*+S181*+N194F+L205Y+Y242F+E259G+N279S+G303R+E390A+G475K, H1*+G7A+T40G+W140Y+R180*+S181*+N194F+L205Y+Y242F+E259G+N279S+G303R+E390A+G475K, L63F+W140Y+L173F+R180*+S181*+N194F+L205Y+Y242F+E259G+N279S+H323W W140Y+L173F+R180*+S181*+N194F+L205Y+N225S+Y242F+E259G+N279S+G303R+H323A, H1*+N3D+G7A+D118T+W140Y+R180*+S181*+T182S+E189P+N194F+L205Y+Y242F+E259G+N279S+K280Y+S302Y+E390A+N474L, H1*+N3D+G7A+D118T+W140Y+R180*+S181*+T182S+E189P+N194F+L205Y+Y242F+E259G+N279S+K280Y+G303R+Y362V+E390A+H420A+N474L, H1*+N3D+G7A+D118T+W140Y+R180*+S181*+T182S+E189P+N194F+L205Y+Y242F+E259G+N279S+K280Y+S302T+V365I+E390A+N474L, H1*+G7A+D118T+W140Y+R180*+S181*+T182S+E189P+N194F+L205Y+Y242F+E259G+N279S+K280Y+S302Y+E390A+H420A+G475K, H1*+G7A+D118T+W140Y+R180*+S181*+T182S+E189P+N194F+L205Y+Y242F+E259G+N279S+K280Y+S302Y+E390A+H420A+G475K, H1*+N3D+G7A+D118T+W140Y+R180*+S181*+T182S+E189P+N194F+L205Y+Y242F+E259G+N279S+K280Y+S302Y+E390A+N474L, H1*+N3D+G7A+D118T+W140Y+R180*+S181*+T182S+E189P+N194F+L205Y+Y242F+E259G+N279S+K280Y+S302T+E390A+H420A+G475K, H1*+G7A+D118T+W140Y+R180*+S181*+T182S+E189P+N194F+L205Y+Y242F+E259G+N279S+K280Y+S302Y+E390A+H420A+G475K, H1*+N3D+G7A+D118T+W140Y+R180*+S181*+T182S+E189P+N194F+L205Y+Y242F+E259G+N279S+K280Y+S302T+E390A+H420A+G475K, H1*+G7A+D118T+W140Y+R180*+S181*+T182S+E189P+N194F+L205Y+Y242F+E259G+N279S+K280Y+G303R+E390A+H420A+G476A, H1*+N3D+G7A+D118T+W140Y+R180*+S181*+T182S+E189P+N194F+L205Y+Y242F+E259G+N279S+K280Y+S302T+E390A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+G7A+D118T+W140Y+R180*+S181*+T182S+E189P+N194F+L205Y+Y242F+E259G+N279S+K280Y+G303R+E390A+H420A+N474L, H1*+N3D+G7A+R37A+D118T+W140Y+R180*+S181*+T182S+E189P+N194F+L205Y+Y242F+E259G+N279S+K280Y+G303R+N394D+N474F, H1*+G7A+D118T+W140Y+R180*+S181*+T182S+E189P+N194F+L205Y+Y242F+E259G+N279S+K280Y+G303R+N394W+H420P+N474L, H1*+G7A+G73L+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+G303R+E390A+G476A, H1*+N3D+G7A+D118T+W140Y+R180*+S181*+T182S+E189P+N194F+L205Y+Y242F+E259G+N279S+K280Y+S302Y+E390A+H420A+G475K, H1*+N3D+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+N394W+G475K, H1*+N3D+G7A+K93R+D118T+W140Y+R180*+S181*+T182S+E189P+N194F+L205Y+Y242F+E259G+N279S+K280Y+G303R+E390A+H420A+N474L, H1*+G7A+R37N+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+G303R+E390D+H420P+G475K, H1*+G7A+D118T+W140Y+R180*+S181*+T182S+E189P+N194F+L205Y+Y242F+E259G+N279S+K280Y+S302T+N394W+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+D118T+W140Y+R180*+S181*+T182S+E189P+N194F+L205Y+Y242F+E259G+N279S+K280Y+G303R+N394W+H420A+G475K, H1*+G7A+R37N+D118T+W140Y+R180*+S181*+T182S+E189P+N194F+L205Y+Y242F+E259G+N279S+K280Y+G303R+E390A+H420A+G475K, H1*+N3D+G7A+D118T+W140Y+R180*+S181*+T182S+E189P+N194F+L205Y+Y242F+E259G+N279S+K280Y+S302T+E390A+G476H, H1*+G7A+D118T+W140Y+R180*+S181*+T182S+E189P+N194F+L205Y+Y242F+E259G+N279S+K280Y+G303R+N394D+H420A+G475K, H1*+N3D+G7A+D118T+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+N279S+K280Y+S302T+E390A+G476A, H1*+G7A+D118T+W140Y+R180*+S181*+T182S+E189P+N194F+L205Y+Y242F+E259G+N279S+K280Y+G303R+E390A+H420P+G475K, H1*+N3D+G7A+D118T+W140Y+R180*+S181*+T182S+E189P+N194F+L205Y+Y242F+E259G+N279S+K280Y+G303R+E390A+H420A+N474L, N3G+G7A+D118T+W140Y+R180*+S181*+T182S+E189P+N194F+L205Y+Y242F+E259G+N279S+K280Y+G303R+E390A+H420A+N474L, H1*+G7A+R37N+D118T+W140Y+R180*+S181*+T182S+E189P+N194F+L205Y+Y242F+E259G+N279S+K280Y+G303R+N394W+H420A+G475K, H1*+N3D+G7A+R37N+D118T+Q138V+W140Y+R180*+S181*+T182S+E189P+N194F+L205Y+Y242F+E259G+N279S+K280Y+G303R+E390A+G475K, H1*+G7A+D118T+W140Y+R180*+S181*+T182S+E189P+N194F+L205Y+Y242F+E259G+N279S+K280Y+S302Y+E390A+G476H, H1*+G7A+D118T+Y135E+W140Y+R180*+S181*+T182S+E189P+N194F+L205Y+Y242F+E259G+N279S+K280Y+S302T+E390A+N474L, H1*+G7A+D118T+W140Y+R180*+S181*+T182S+E189P+N194F+L205Y+Y242F+E259G+N279S+K280Y+G303R+N394W+H420A+G475K, H1*+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+G303R+E390A+H420P+G475K, H1*+G7A+V103I+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+M260D+N279S+K280Y+S302Y+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+G303R+H323W+E390A+N474L, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+P146H+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+F261P+N279S+K280Y+G303R+E390A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+Q98L+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+G303R+E390A+N474L, H1*+G7A+D118T+W140Y+R180*+S181*+T182S+E189P+N194F+L205Y+Y242F+E259G+N279S+K280Y+G303R+P321H+E390A+G475K, H1*+G7A+D118T+W140Y+R180*+S181*+T182S+E189P+N194F+L205Y+Y242F+E259G+N279S+K280Y+G303R+P321H+E390A+N474L, H1*+G7A+D118T+S132D+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+G303R+N394D+R414M+G475K, H1*+G7A+T40G+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+T224F+Y242F+E259G+N279S+K280Y+G303R+N394W+H420A+G475K, H1*+G7A+D118T+W140Y+R180*+S181*+T182S+E189P+N194F+L205Y+Y242F+E259G+M260D+N279S+K280Y+S302T+E390A+G475K, H1*+G7A+D118T+W140Y+R180*+S181*+T182S+E189P+N194F+L205Y+Y242F+E259G+M260W+N279S+K280Y+S302Y+E390A+G476A, H1*+G7A+D118T+Y135E+W140Y+R180*+S181*+T182S+E189P+N194F+L205Y+Y242F+E259G+M260D+N279S+K280Y+G303R+W346N+E390A+G475K, H1*+G7A+D118T+W140Y+R180*+S181*+T182S+E189P+N194F+L205Y+Y242F+E259G+N279S+K280Y+G303R+P321H+Q360Y+E390A+G476A, H1*+G7A+D118T+W140Y+R180*+S181*+T182S+E189P+N194F+L205Y+Y242F+E259G+M260D+N279S+K280Y+S302Y+E390A+N470M+G475K, H1*+G7A+D118T+W140Y+R180*+S181*+T182S+E189P+N194F+L205Y+Y242F+E259G+M260W+N279S+K280Y+G303R+E390A+G476A, H1*+G7A+D118T+W140Y+R180*+S181*+T182S+E189P+N194F+L205Y+T224Y+Y242F+N279S+K280Y+G303R+E390A+G476A, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+N279S+K280Y+S302Y+E390A+N474F, H1*+G7A+D118T+W140Y+R180*+S181*+T182S+E189P+N194F+L205Y+Y242F+E259G+M260W+N279S+K280Y+G303R+E390A+G476A, H1*+G7A+D118T+W140Y+R180*+S181*+T182S+E189P+N194F+L205Y+Y242F+E259G+M260W+N279S+K280Y+S302Y+E390A+G475K, H1*+N3D+G7A+D118T+W140Y+R180*+S181*+T182S+E189P+N194F+L205Y+Y242F+E259G+N279S+K280Y+S302T+E390A+H420A+G475K, H1*+G7A+D118T+W140Y+R180*+S181*+T182S+E189P+N194F+L205Y+T224Y+Y242F+E259G+N279S+K280Y+G303R+E390A+N474L, H1*+G7A+D118T+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+E390A+H420A+G475K, H1*+G7A+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+S302T+E390A+N474L, H1*+G7A+D118T+W140Y+P146H+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+G475K, H1*+N3G+G7A+D118T+W140Y+R180*+S181*+T182S+E189P+N194F+L205Y+Y242F+E259G+N279S+K280Y+S302T+E390A+G475K, H1*+G7A+D118T+S132L+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+G475K, H1*+D118T+W140Y+R180*+S181*+T182S+E189P+N194F+L205Y+Y242F+E259G+N279S+K280Y+G303R+E390A+G475K, H1*+G7A+W140Y+R180*+S181*+T182S+E189P+N194F+L205Y+Y242F+E259G+N279S+K280Y+G303R+E390A+G475K, H1*+G7A+D118T+R180*+S181*+T182S+E189P+N194F+L205Y+Y242F+E259G+N279S+K280Y+G303R+E390A+G475K, H1*+G7A+D118T+W140Y+R180*+S181*+E189P+N194F+L205Y+Y242F+E259G+N279S+K280Y+G303R+E390A+G475K, H1*+G7A+D118T+W140Y+R180*+S181*+T182S+N194F+L205Y+Y242F+E259G+N279S+K280Y+G303R+E390A+G475K, H1*+G7A+D118T+W140Y+R180*+S181*+T182S+E189P+L205Y+Y242F+E259G+N279S+K280Y+G303R+E390A+G475K, H1*+G7A+D118T+W140Y+R180*+S181*+T182S+E189P+N194F+Y242F+E259G+N279S+K280Y+G303R+E390A+G475K, H1*+G7A+D118T+W140Y+R180*+S181*+T182S+E189P+N194F+L205Y+E259G+N279S+K280Y+G303R+E390A+G475K, H1*+G7A+D118T+W140Y+R180*+S181*+T182S+E189P+N194F+L205Y+Y242F+N279S+K280Y+G303R+E390A+G475K, H1*+G7A+D118T+W140Y+R180*+S181*+T182S+E189P+N194F+L205Y+Y242F+E259G+K280Y+G303R+E390A+G475K, H1*+G7A+D118T+W140Y+R180*+S181*+T182S+E189P+N194F+L205Y+Y242F+E259G+N279S+G303R+E390A+G475K, H1*+G7A+D118T+W140Y+R180*+S181*+T182S+E189P+N194F+L205Y+Y242F+E259G+N279S+K280Y+E390A+G475K, H1*+G7A+D118T+W140Y+R180*+S181*+T182S+E189P+N194F+L205Y+Y242F+E259G+N279S+K280Y+G303R+G475K, H1*+G7A+D118T+W140Y+R180*+S181*+T182S+E189P+N194F+L205Y+Y242F+E259G+N279S+K280Y+G303R+E390A, H1*+G7A+D118T+W140Y+R180*+S181*+T182S+E189P+N194F+L205V+Y242F+E259G+N279S+K280Y+G303R+E390A+G475K, H1*+G7A+D118T+W140Y+R180*+S181*+T182S+E189P+N194F+L205N+Y242F+E259G+N279S+K280Y+G303R+E390A+G475K, H1*+G7A+D118T+W140Y+R180*+S181*+T182S+E189P+N194F+L205Q+Y242F+E259G+N279S+K280Y+G303R+E390A+G475K, H1*+G7A+D118T+W140Y+R180*+S181*+T182S+A185Q+E189P+N194F+L205Y+Y242F+E259G+N279S+K280Y+G303R+E390A+G475K, H1*+G7A+D118T+W140Y+R180*+S181*+T182S+E189P+N194F+L205Y+Y242F+E259G+F266Y+N279S+K280Y+G303R+E390A+G475K, H1*+G7A+D118T+W140Y+R180*+S181*+A185Q+E189P+N194F+L205Y+Y242F+E259G+N279S+K280Y+G303R+E390A+G475K, H1*+G7A+D118T+W140Y+R180*+S181*+T182S+A185Q+E189P+N194F+L205Y+Y242F+E259G+F266Y+N279S+K280Y+G303R+E390A+G475K, H1*+G7A+D118T+W140Y+R180*+S181*+T182S+A185Q+E189P+N194F+L205N+Y242F+E259G+F266Y+N279S+K280Y+G303R+E390A+G475K, H1*+G7A+D118T+W140Y+R180*+S181*+T182S+A185D+E189P+N194F+Y242F+E259G+F266Y+N279S+K280Y+G303R+E390A+G475K, H1*+G7A+D118T+W140Y+R180*+S181*+T182S+A185D+E189P+Y242F+S243Q+E259G+F266Y+N279S+K280Y+G303R+E390A+G475K, H1*+G7A+D118T+W140Y+R180Q+S181*+T182*+A185Q+E189P+Y242F+S243Q+E259G+F266Y+N279S+K280Y+G303R+E390A+G475K, H1*+G7A+D118T+W140Y+R180*+S181*+T182S+A185Q+E189P+Y242F+E259G+F266Y+N279S+K280Y+G303R+E390A+G475K, H1*+G7A+D118T+W140Y+R180*+S181*+T182S+A185Q+E189P+L205Y+Y242F+E259G+F266Y+N279S+K280Y+G303R+E390A+G475K, H1*+G7A+D118T+W140Y+R180*+S181*+T182S+A185D+E189P+L205Y+Y242F+E259G+F266Y+N279S+K280Y+G303R+E390A+G475K, H1*+G7A+D118T+W140Y+R180Q+S181*+T182*+A185Q+E189P+N194F+L205Y+Y242F+E259G+F266Y+N279S+K280Y+G303R+E390A+G475K, H1*+N3D+G7A+D118T+W140Y+R180*+S181*+T182S+E189P+N194F+L205Y+Y242F+E259G+N279S+K280Y+S302T+E390A+H420A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+T40G+D118T+W140Y+R180*+S181*+T182S+E189P+N194F+L205Y+Y242F+E259G+N279S+K280Y+S302Y+E390A+G475K, H1*+G7A+T40G+D118T+W140Y+R180*+S181*+T182S+E189P+N194F+L205Y+Y242F+E259G+N279S+K280Y+G303R+E390A+G476A, H1*+G7A+T40G+D118T+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+G303R+N394D+N422V+G475K, H1*+G7A+Q98L+D118T+W140Y+R180*+S181*+T182S+E189P+N194F+L205Y+Y242F+E259G+N279S+K280Y+G303R+N394D+H420A+G475K, H1*+G7A+D118T+W140Y+R180*+S181*+T182S+E189P+N194F+L205Y+Y242F+E259G+M260D+N279S+K280Y+G303R+Y362V+E390A+G475K, H1*+G7A+D118T+Y135E+W140Y+R180*+S181*+T182S+E189P+N194F+L205Y+Y242F+E259G+F261P+N279S+K280Y+G303R+Y362V+E390A+G475K, H1*+G7A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+Y135E+W140Y+R180*+S181*+T182S+E189P+N194F+L205Y+T224F+Y242F+E259G+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+E259G+F261P+N279S+K280Y+G303R+E390A+G475K, H1*+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+Y371N+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+N474F, H1*+G7A+D118T+Y135E+W140Y+R180*+S181*+T182S+E189P+N194F+L205Y+Y242F+E259G+N279S+K280Y+G303R+P321Y+E390A+G475K, H1*+G7A+D118T+S132D+W140Y+R180*+S181*+T182S+E189P+N194F+L205Y+Y242F+E259G+N279S+K280Y+G303R+P321Y+E390A+G475K, H1*+N3G+G7A+D118T+Q138V+W140Y+R180*+S181*+T182S+E189P+N194F+L205Y+Y242F+E259G+N279S+K280Y+G303R+E390A+H420A+G476A, H1*+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+T256D+N279S+K280Y+G303R+E390A+N470M+G475K, H1*+N3D+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+S132D+W140Y+R180*+S181*+T182S+E189P+N194F+L205Y+Y242F+E259G+N279S+K280Y+G303R+Y362V+E390A+N474L, H1*+G7A+D118T+W140Y+R180*+S181*+T182S+E189P+N194F+L205Y+Y242F+E259G+N279S+K280Y+S302T+E390A+G475K, H1*+G7A+D118T+Q138V+W140Y+R180*+S181*+T182S+E189P+N194F+L205Y+Y242F+E259G+N279S+K280Y+G303R+E390A+G476A, H1*+G7A+D118T+W140Y+R180*+S181*+T182S+E189P+N194F+L205Y+Y242F+E259G+N279S+K280Y+S302T+Y362V+E390A+G475K, H1*+G7A+D118T+W140Y+R180*+S181*+T182S+E189P+N194F+L205Y+Y242F+E259G+N279S+K280Y+G303R+Y362V+E390A+G476A, H1*+G7A+D118T+W140Y+R180*+S181*+T182S+E189P+N194F+L205Y+Y242F+E259G+M260D+N279S+K280Y+G303R+E390A+N474L, H1*+G7A+D118T+W140Y+R180*+S181*+T182S+E189P+N194F+L205Y+T224Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+Y362V+E390A+G475K, H1*+G7A+D118T+W140Y+R180*+S181*+T182S+E189P+N194F+L205Y+Y242F+E259G+M260W+N279S+K280Y+S302T+E390A+G475K, H1*+G7A+D118T+W140Y+R180*+S181*+T182S+E189P+N194F+L205Y+T224Y+Y242F+E259G+N279S+K280Y+G303R+E390A+G476A, H1*+G7A+D118T+W140Y+R180*+S181*+T182S+E189P+N194F+L205Y+Y242F+E259G+M260D+N279S+K280Y+G303R+E390A+N474F, H1*+G7A+D118T+W140Y+R180*+S181*+T182S+E189P+N194F+L205Y+Y242F+E259G+N279S+K280Y+S302Y+E390A+N474L, H1*+G7A+D118T+W140Y+R180*+S181*+T182S+E189P+N194F+L205Y+Y242F+E259G+N279S+K280Y+S302Y+E390A+G476H, H1*+G7A+D118T+W140Y+R180*+S181*+T182S+E189P+N194F+L205Y+Y242F+E259G+M260D+N279S+K280Y+G303R+E390A+H420A+G475K, H1*+G7A+D118T+Y135E+W140Y+R180*+S181*+T182S+E189P+N194F+L205Y+Y242F+E259G+N279S+K280Y+G303R+N394W+H420A+G475K, H1*+N3D+G7A+D118T+W140Y+R180*+S181*+T182S+E189P+N194F+L205Y+Y242F+E259G+N279S+K280Y+G303R+N394D+H420A+G475K, H1*+G7A+D118T+Q138V+W140Y+R180*+S181*+T182S+E189P+N194F+L205Y+Y242F+E259G+N279S+K280Y+G303R+E390A+H420Q+G475K, H1*+G7A+R37N+D118T+W140Y+R180*+S181*+T182S+E189P+N194F+L205Y+Y242F+E259G+N279S+K280Y+G303R+E390A+H420P+G476H, H1*+G7A+R37A+Q98L+D118T+W140Y+R180*+S181*+T182S+E189P+N194F+L205Y+Y242F+E259G+N279S+K280Y+S302Y+E390A+G475K, H1*+G7A+R37A+D118T+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+N279S+K280Y+G303R+E390A+H420P+G475K, H1*+G7A+D118T+W140Y+R180*+S181*+T182S+E189P+N194F+L205Y+Y242F+E259G+N279S+K280Y+G303R+E390A+N470M+G476A, H1*+G7A+D118T+W140Y+R180*+S181*+T182S+E189P+N194F+L205Y+Y242F+E259G+M260D+N279S+K280Y+G303R+E390A+H420P+G476A, H1*+G7A+D118T+W140Y+R180*+S181*+T182S+E189P+N194F+L205Y+Y242F+E259G+N279S+K280Y+S302T+E390A+H420A+G475K, H1*+G7A+R37A+D118T+W140Y+R180*+S181*+T182S+E189P+N194F+L205Y+Y242F+E259G+N279S+K280Y+S302Y+E390A+H420A+G475K, H1*+G7A+D118T+Y135E+W140Y+R180*+S181*+T182S+E189P+N194F+L205Y+Y242F+E259G+N279S+K280Y+S302T+Y371N+E390A+G475K, H1*+G7A+R37N+D118T+S132D+W140Y+R180*+S181*+T182S+E189P+N194F+L205Y+Y242F+E259G+N279S+K280Y+G303R+E390A+N470M+G475K, H1*+G7A+D118T+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+N279S+K280Y+G303R+H323W+E390A+H420A+N474L, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+N394W+H420P+G475K, H1*+G7A+D118T+W140Y+R180*+S181*+T182S+E189P+N194F+L205Y+Y242F+E259G+N279S+K280Y+S302T+H323W+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+Y135E+W140Y+R180*+S181*+T182S+E189P+N194F+L205Y+Y242F+E259G+N279S+K280Y+G303R+E390A+R414M+G476H, H1*+G7A+R37N+D118T+W140Y+R180*+S181*+T182S+E189P+N194F+L205Y+Y242F+E259G+N279S+K280Y+S302Y+E390A+H420A+G475K, H1*+G7A+R37A+D118T+W140Y+R180*+S181*+T182S+E189P+N194F+L205Y+Y242F+E259G+N279S+K280Y+S302T+E390A+H420A+G476A, H1*+G7A+R37N+D118T+W140Y+R180*+S181*+T182S+E189P+N194F+L205Y+Y242F+E259G+N279S+K280Y+S302Y+E390A+H420A+G475K, H1*+G7A+R37A+D118T+W140Y+R180*+S181*+T182S+E189P+N194F+L205Y+Y242F+E259G+N279S+K280Y+S302Y+E390A+N474L, H1*+G7A+R37A+D118T+W140Y+R180*+S181*+T182S+E189P+N194F+L205Y+Y242F+E259G+N279S+K280Y+S302Y+E390A+G475K, H1*+N3D+G7A+R37A+D118T+W140Y+R180*+S181*+T182S+E189P+N194F+L205Y+Y242F+E259G+N279S+K280Y+S302Y+N394D+H420A+G475K, H1*+G7A+R37A+D118T+W140Y+R180*+S181*+T182S+E189P+N194F+L205Y+Y242F+E259G+N279S+K280Y+S302Y+E390A+H420A+G476H, H1*+G7A+R37A+D118T+W140Y+R180*+S181*+T182S+E189P+N194F+L205Y+Y242F+E259G+N279S+K280Y+S302Y+N394D+H420A+G475K, H1*+G7A+R37A+D118T+W140Y+R180*+S181*+T182S+E189P+N194F+L205Y+Y242F+E259G+N279S+K280Y+S302Y+E390A+N474L, H1*+G7A+R37N+D118T+W140Y+R180*+S181*+T182S+E189P+N194F+L205Y+Y242F+E259G+N279S+K280Y+S302T+E390A+H420A+G475K, H1*+G7A+R37A+D118T+W140Y+R180*+S181*+T182S+E189P+N194F+L205Y+Y242F+E259G+N279S+K280Y+S302Y+E390A+H420A+G475K, H1*+G7A+R37N+D118T+W140Y+R180*+S181*+T182S+E189P+N194F+L205Y+Y242F+E259G+N279S+K280Y+S302T+E390A+H420A+G475K, H1*+G7A+R37A+D118T+W140Y+R180*+S181*+T182S+E189P+N194F+L205Y+Y242F+E259G+N279S+K280Y+S302T+E390A+H420A+G475K, H1*+G7A+D118T+W140Y+R180*+S181*+T182S+E189P+N194F+L205Y+Y242F+E259G+N279S+K280Y+S302T+N394W+H420A+G475K, H1*+G7A+R37A+D118T+W140Y+R180*+S181*+T182S+E189P+N194F+L205Y+Y242F+E259G+N279S+K280Y+S302Y+N394D+G475K, H1*+G7A+R37N+D118T+W140Y+R180*+S181*+T182S+E189P+N194F+L205Y+Y242F+E259G+N279S+K280Y+S302Y+E390A+H420A+G476H, H1*+G7A+R37N+D118T+W140Y+R180*+S181*+T182S+E189P+N194F+L205Y+Y242F+E259G+N279S+K280Y+S302T+N394D+G475K, H1*+G7A+R37A+D118T+W140Y+R180*+S181*+T182S+E189P+N194F+L205Y+Y242F+E259G+N279S+K280Y+S302T+E390A+G475K, H1*+G7A+R37N+D118T+W140Y+R180*+S181*+T182S+E189P+N194F+L205Y+Y242F+E259G+N279S+K280Y+S302T+N394D+H420A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+D118T+W140Y+R180*+S181*+T182S+E189P+N194F+L205Y+Y242F+E259G+N279S+K280Y+S302T+E390A+G475K, H1*+N3G+G7A+D118T+W140Y+R180*+S181*+T182S+E189P+N194F+L205Y+Y242F+E259G+N279S+K280Y+S302T+E390A+H420A+G475K, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302Y+E390A+N474L, H1*+N3D+G7A+D118T+W140Y+R180*+S181*+T182S+E189P+N194F+L205Y+Y242F+E259G+N279S+K280Y+S302T+E390A+G475K, H1*+N3D+G7A+D118T+W140Y+R180*+S181*+T182S+E189P+N194F+L205Y+Y242F+E259G+N279S+K280Y+S302Y+E390A+G475K, H1*+N3D+G7A+D118T+W140Y+R180*+S181*+T182S+E189P+N194F+L205Y+Y242F+E259G+N279S+K280Y+S302Y+E390A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3D+G7A+R37A+D118T+W140Y+R180*+S181*+T182S+E189P+N194F+L205Y+Y242F+E259G+N279S+K280Y+G303R+E390A+G475K, H1*+N3G+G7A+R37N+D118T+W140Y+R180*+S181*+T182S+E189P+N194F+L205Y+Y242F+E259G+N279S+K280Y+G303R+N394W+H420A+G475K, H1*+N3D+G7A+R37N+D118T+W140Y+R180*+S181*+T182S+E189P+N194F+L205Y+Y242F+E259G+N279S+K280Y+G303R+E390A+H420A+G475K, H1*+N3D+G7A+R37N+D118T+W140Y+R180*+S181*+T182S+E189P+N194F+L205Y+Y242F+E259G+N279S+K280Y+G303R+G361M+H420A+G475K, H1*+N3D+G7A+R37A+D118T+W140Y+R180*+S181*+T182S+E189P+N194F+L205Y+Y242F+E259G+N279S+K280Y+G303R+E390A+H420A+G475K, H1*+G7A+R37A+D118T+W140Y+R180*+S181*+T182S+E189P+N194F+L205Y+Y242F+E259G+M260D+N279S+K280Y+G303R+N394W+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+R37A+D118T+W140Y+R180*+S181*+T182S+E189P+N194F+L205Y+T224F+Y242F+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+N394D+G475K, H1*+G7A+R37A+D118T+W140Y+R180*+S181*+T182S+E189P+N194F+L205Y+Y242F+E259G+M260D+N279S+K280Y+G303R+N394D+G475K, H1*+G7A+R37N+D118T+W140Y+R180*+S181*+T182S+E189P+N194F+L205Y+Y242F+E259G+M260D+N279S+K280Y+G303R+N394D+G475K, H1*+G7A+D118T+W140Y+R180*+S181*+T182S+E189P+N194F+L205Y+Y242F+E259G+M260D+N279S+K280Y+G303R+H420A+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+R37N+D118T+W140Y+R180*+S181*+T182S+E189P+N194F+L205Y+T224Y+Y242F+N279S+K280Y+G303R+E390A+G475K, H1*+G7A+D118T+W140Y+R180*+S181*+T182S+E189P+N194F+L205Y+Y242F+E259G+N279S+K280Y+G303R+N394W+H420A+G475K, H1*+G7A+D118T+W140Y+R180*+S181*+T182S+E189P+N194F+L205Y+Y242F+E259G+N279S+K280Y+G303R+N394W+H420A+N474L, H1*+G7A+D118T+W140Y+P146H+R180*+S181*+T182S+E189P+N194F+L205Y+Y242F+E259G+N279S+K280Y+S302T+E390A+G476A, H1*+G7A+D118T+W140Y+P146H+R180*+S181*+T182S+E189P+N194F+L205Y+Y242F+E259G+N279S+K280Y+S302Y+E390A+N474L, H1*+G7A+D118T+W140Y+R180*+S181*+T182S+A185Q+E189P+N194F+Y242F+E259G+F266Y+N279S+K280Y+G303R+E390A+G475K, H1*+G7A+D118T+W140Y+R180*+S181*+T182S+A185Q+E189P+N194F+L205Q+Y242F+E259G+F266Y+N279S+K280Y+G303R+E390A+G475K, H1*+G7A+D118T+W140Y+R180Q+S181*+T182*+A185Q+E189P+N194F+Y242F+E259G+F266Y+N279S+K280Y+G303R+E390A+G475K, H1*+G7A+D118T+W140Y+R180*+S181*+T182S+A185Q+E189P+Y242F+S243Q+E259G+F266Y+N279S+K280Y+G303R+E390A+G475K, H1*+N3G+G7A+V104M+D118T+W140Y+R180*+S181*+T182S+E189P+N194F+L205Y+Y242F+E259G+N279S+K280Y+S302Y+N394W+G475K, H1*+N3D+G7A+D118T+W140Y+R180*+S181*+T182S+E189P+N194F+L205Y+Y242F+E259G+N279S+K280Y+G303R+E390A+G475K, H1*+N3G+G7A+D118T+W140Y+R180*+S181*+T182S+E189P+N194F+L205Y+Y242F+E259G+N279S+K280Y+G303R+N394W+H420A+G475K, H1*+N3D+G7A+D118T+W140Y+R180*+S181*+T182S+E189P+N194F+L205Y+Y242F+E259G+N279S+K280Y+G303R+E390A+H420A+G475K, H1*+N3D+G7A+D118T+S132D+W140Y+R180*+S181*+T182S+E189P+N194F+L205Y+Y242F+E259G+N279S+K280Y+G303R+E390A+H420A+G475K, H1*+G7A+D118T+W140Y+R180*+S181*+T182S+E189P+N194F+L205Y+Y242F+E259G+N279S+K280Y+G303R+E390A+N470M+G475K, H1*+G7A+D118T+W140Y+R180*+S181*+T182S+E189P+N194F+L205Y+Y242F+E259G+N279S+K280Y+S302Y+Y371N+E390A+G475K, H1*+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+G475K, H1*+G7A+D118T+Y135E+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+T224F+Y242F+E259G+N279S+K280Y+S302T+E390A+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+N394W+G475K, H1*+G7A+D118T+W140Y+R180*+S181*+T182S+E189P+N194F+L205Y+Y242F+E259G+M260D+N279S+K280Y+G303R+E390A+G475K, H1*+G7A+R37A+D118T+W140Y+R180*+S181*+T182S+E189P+N194F+L205Y+Y242F+E259G+M260W+N279S+K280Y+G303R+E390A+N470M+G475K, H1*+G7A+R37N+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+E390A+G475K, H1*+G7A+D118T+W140Y+R180*+S181*+T182S+E189P+N194F+L205Y+Y242F+E259G+M260D+N279S+K280Y+G303R+N394D+G475K, H1*+G7A+R37N+D118T+W140Y+R180*+S181*+T182S+E189P+N194F+L205Y+Y242F+E259G+M260W+N279S+K280Y+G303R+E390A+H420A+G475K, H1*+G7A+D118T+W140Y+R180*+S181*+T182S+E189P+N194F+L205Y+Y242F+E259G+M260D+N279S+K280Y+G303R+E390A+N470M+G475K, H1*+G7A+R37A+D118T+W140Y+R180*+S181*+T182S+E189P+N194F+L205Y+Y242F+E259G+M260D+N279S+K280Y+G303R+N394W+G475K, H1*+G7A+R37A+D118T+W140Y+R180*+S181*+T182S+E189P+N194F+L205Y+Y242F+E259G+N279S+K280Y+G303R+E390A+H420A+G475K, H1*+G7A+R37A+D118T+W140Y+R180*+S181*+T182S+E189P+N194F+L205Y+Y242F+E259G+M260D+N279S+K280Y+G303R+E390A+G475K, H1*+G7A+R37A+D118T+W140Y+R180*+S181*+T182S+E189P+N194F+L205Y+T224Y+Y242F+E259G+N279S+K280Y+G303R+N394W+G475K, H1*+G7A+R37N+D118T+W140Y+R180*+S181*+T182S+E189P+N194F+L205Y+Y242F+E259G+M260D+N279S+K280Y+G303R+N394W+H420A+G475K, H1*+G7A+R37A+D118T+W140Y+R180*+S181*+T182S+E189P+N194F+L205Y+Y242F+E259G+M260D+N279S+K280Y+G303R+E390A+N470M+G475K, H1*+G7A+R37A+D118T+W140Y+R180*+S181*+T182S+E189P+N194F+L205Y+Y242F+T256D+N279S+K280Y+G303R+E390A+G475K, H1*+G7A+R37A+D118T+W140Y+R180*+S181*+T182S+E189P+N194F+L205Y+Y242F+E259G+F261P+N279S+K280Y+G303R+E390A+G475K, H1*+G7A+R37A+D118T+W140Y+R180*+S181*+T182S+E189P+N194F+L205Y+Y242F+E259G+M260D+N279S+K280Y+G303R+N394D+H420A+G475K, H1*+G7A+R37A+D118T+W140Y+R180*+S181*+T182S+E189P+N194F+L205Y+Y242F+E259G+M260D+N279S+K280Y+G303R+N394D+G475K, H1*+G7A+R37N+D118T+W140Y+N174K+R180*+S181*+T182S+E189P+N194F+L205Y+Y242F+E259G+M260D+N279S+K280Y+G303R+N394W+G475K, H1*+G7A+R37A+D118T+W140Y+R180*+S181*+T182S+E189P+N194F+L205Y+T224Y+Y242F+E259G+N279S+K280Y+G303R+E390A+G475K, H1*+G7A+R37A+D118T+W140Y+R180*+S181*+T182S+E189P+N194F+L205Y+T224Y+Y242F+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+H420A+G475K, H1*+N3D+G7A+D118T+W140Y+R180*+S181*+T182S+E189P+N194F+L205Y+Y242F+E259G+M260D+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3D+G7A+D118T+W140Y+R180*+S181*+T182S+E189P+N194F+L205Y+T224Y+Y242F+E259G+N279S+K280Y+G303R+E390A+G475K, H1*+N3G+G7A+R37N+D118T+W140Y+R180*+S181*+T182S+E189P+N194F+L205Y+Y242F+E259G+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+N3D+G7A+D118T+W140Y+R180*+S181*+T182S+E189P+N194F+L205Y+Y242F+T256D+N279S+K280Y+G303R+E390A+G475K, H1*+G7A+D118T+W140Y+R180*+S181*+T182S+E189P+N194F+L205Y+Y242F+E259G+N279S+K280Y+S302T+E390A+N470M+G475K, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+S132L+W140Y+R180*+S181*+T182S+E189P+N194F+L205Y+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+H420A+G476A, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302Y+E390A+G475K, H1*+G7A+D118T+W140Y+R180*+S181*+T182S+E189P+N194F+L205Y+Y242F+E259G+N279S+K280Y+S302Y+N394D+G475K, H1*+G7A+D118T+W140Y+R180*+S181*+T182S+E189P+N194F+L205Y+T224F+Y242F+E259G+N279S+K280Y+G303R+E390A+H420A+G475K, H1*+G7A+D118T+W140Y+R180*+S181*+T182S+E189P+N194F+L205Y+T224F+Y242F+E259G+N279S+K280Y+S302Y+E390A+G475K, H1*+G7A+D118T+W140Y+R180*+S181*+T182S+E189P+N194F+L205Y+T224Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302Y+E390A+G475K, H1*+G7A+D118T+S132D+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+N394W+G475K, H1*+G7A+D118T+S132L+W140Y+R180*+S181*+T182S+E189P+N194F+L205Y+T224F+Y242F+E259G+N279S+K280Y+S302Y+E390A+G475K, H1*+G7A+D118T+Q138V+W140Y+R180*+S181*+T182S+E189P+N194F+L205Y+Y242F+E259G+N279S+K280Y+S302Y+N394D+G475K, H1*+G7A+D118T+S132L+W140Y+R180*+S181*+T182S+E189P+N194F+L205Y+Y242F+E259G+N279S+K280Y+S302Y+N394W+G475K, H1*+N3D+G7A+D118T+W140Y+R180*+S181*+T182S+E189P+N194F+L205Y+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+G303R+E390A+H420A+G476H, H1*+G7A+D118T+W140Y+R180*+S181*+T182S+E189P+N194F+L205Y+T224F+Y242F+E259G+N279S+K280Y+S302T+E390A+G475K, H1*+G7A+D118T+W140Y+R180*+S181*+T182S+E189P+N194F+L205Y+Y242F+E259G+N279S+K280Y+S302T+N394D+G475K, H1*+G7A+D118T+Q138V+W140Y+R180*+S181*+T182S+E189P+N194F+L205Y+Y242F+E259G+N279S+K280Y+S302Y+E390A+G475K, H1*+G7A+D118T+Q138V+W140Y+R180*+S181*+T182S+E189P+N194F+L205Y+Y242F+E259G+N279S+K280Y+S302Y+E390A+G475K, H1*+G7A+D118T+S132L+W140Y+R180*+S181*+T182S+E189P+N194F+L205Y+Y242F+E259G+N279S+K280Y+S302Y+N394W+G475K, H1*+G7A+D118T+Q138V+W140Y+R180*+S181*+T182S+E189P+N194F+L205Y+T224F+Y242F+E259G+N279S+K280Y+S302Y+E390A+G475K, H1*+G7A+Q98L+D118T+W140Y+R180*+S181*+T182S+E189P+N194F+L205Y+Y242F+E259G+N279S+K280Y+G303R+E390A+G475K, H1*+G7A+R37N+D118T+W140Y+R180*+S181*+T182S+E189P+N194F+L205Y+Y242F+T256D+E259G+N279S+K280Y+G303R+E390A+G475K, H1*+G7A+R37A+D118T+W140Y+R180*+S181*+T182S+E189P+N194F+L205Y+Y242F+E259G+N279S+K280Y+G303R+E390A+G475K, H1*+G7A+R37N+D118T+W140Y+R180*+S181*+T182S+E189P+N194F+L205Y+Y242F+E259G+M260D+N279S+K280Y+G303R+N394D+G475K, N3G+G7A+R37V+D118T+W140M+R180*+S181*+T182S+E189P+N194F+L205Y+Y242F+E259G+N279S+K280Y+G303R+E390A+H420A+G475K, H1*+G7A+R37N+D118T+W140Y+R180*+S181*+T182S+E189P+N194F+L205Y+Y242F+E259G+N279S+K280Y+G303R+H323W+N394D+G475K, H1*+G7A+R37A+D118T+W140Y+R180*+S181*+T182S+E189P+N194F+L205Y+Y242F+E259G+N279S+K280Y+G303R+P321Y+L354V+N394D+G475K, H1*+G7A+R37N+D118T+W140Y+R180*+S181*+T182S+E189P+N194F+L205Y+Y242F+E259G+N279S+K280Y+G303R+H323W+Q360M+E390A+G475K, H1*+N3D+G7A+D118T+W140Y+R180*+S181*+T182S+E189P+N194F+L205Y+Y242F+E259G+N279S+K280Y+G303R+H323W+E390A+G475K, H1*+N3D+G7A+D118T+W140Y+R180*+S181*+T182S+E189P+N194F+L205Y+Y242F+E259G+N279S+K280Y+G303R+H323W+E390A+G475K, H1*+G7A+D118T+W140Y+R180*+S181*+T182S+A185D+E189P+Y242F+E259G+F266Y+N279S+K280Y+G303R+E390A+G475K, H1*+G7A+D118T+W140Y+R180Q+S181*+T182*+A185Q+E189P+Y242F+E259G+F266Y+N279S+K280Y+G303R+E390A+G475K, H1*+G7A+D118T+W140Y+R180*+S181*+T182S+E189P+N194F+Y242F+E259G+N279S+K280Y+S302T+E390A+G475K, H1*+G7A+D118T+W140Y+R180*+S181*+T182S+E189P+N194F+Y242F+E259G+M260D+N279S+K280Y+S302T+E390A+G475K, H1*+G7A+R37N+D118T+W140Y+R180*+S181*+T182S+E189P+N194F+Y242F+E259G+N279S+K280Y+S302Y+E390A+G475K, H1*+G7A+D118T+W140Y+R180*+S181*+T182S+E189P+N194F+Y242F+E259G+M260W+N279S+K280Y+G303R+E390A+G476A, H1*+G7A+W140Y+R180*+S181*+E189P+N194F+Y242F+E259G+F266Y+N279S+K280Y+S302T+E390A+G475K, H1*+G7A+D118T+W140Y+R180*+S181*+T182D+E189P+N194F+Y242F+E259G+N279S+K280Y+G303R+E390A+G476A, H1*+G7A+D118T+S132L+W140Y+R180*+S181*+T182D+E189P+N194F+L205Y+Y242F+E259G+M260D+N279S+K280Y+G303R+E390A+G475K, H1*+G7A+D118T+W140Y+R180*+S181*+T182D+E189P+N194F+Y242F+E259G+M260D+N279S+K280Y+S302T+E390A+G475K, H1*+N3D+G7A+D118T+W140Y+R180*+S181*+T182D+E189P+N194F+Y242F+E259G+N279S+K280Y+S302T+E390A+H420A+G475K, H1*+G7A+R37N+D118T+W140Y+R180*+S181*+T182D+E189P+N194F+Y242F+E259G+N279S+K280Y+S302Y+E390A+G475K, H1*+G7A+D118T+W140Y+R180*+S181*+T182S+A185Q+E189P+N194F+L205Y+T224Y+Y242F+N279S+K280Y+G303R+E390A+G476A, H1*+G7A+D118T+W140Y+R180*+S181*+T182S+A185Q+E189P+N194F+Y242F+E259G+M260D+N279S+K280Y+S302Y+E390A+N470M+G475K, H1*+G7A+D118T+W140Y+R180*+S181*+T182S+A185Q+E189P+N194F+Y242F+E259G+N279S+K280Y+G303R+E390A+G476A, H1*+G7A+D118T+W140Y+R180*+S181*+T182S+A185Q+E189P+N194F+Y242F+E259G+M260D+N279S+K280Y+S302T+E390A+G475K, H1*+N3D+G7A+D118T+W140Y+R180*+S181*+T182S+A185Q+E189P+N194F+Y242F+E259G+N279S+K280Y+S302T+E390A+H420A+G475K, H1*+G7A+R37N+D118T+W140Y+R180*+S181*+T182S+A185Q+E189P+N194F+Y242F+E259G+N279S+K280Y+S302Y+E390A+G475K, H1*+G7A+D118T+W140Y+R180*+S181*+T182S+A185Q+E189P+N194F+L205Y+Y242F+E259G+M260W+N279S+K280Y+G303R+E390A+G476A, H1*+G7A+D118T+W140Y+R180*+S181*+T182S+E189P+N194F+T224Y+Y242F+F266Y+N279S+K280Y+G303R+E390A+G476A, H1*+G7A+D118T+W140Y+R180*+S181*+T182S+E189P+N194F+Y242F+E259G+M260D+F266Y+N279S+K280Y+S302Y+E390A+N470M+G475K, H1*+G7A+D118T+W140Y+R180*+S181*+T182S+E189P+N194F+Y242F+E259G+F266Y+N279S+K280Y+S302T+E390A+G475K, H1*+G7A+D118T+W140Y+R180*+S181*+T182S+E189P+N194F+Y242F+E259G+F266Y+N279S+K280Y+G303R+E390A+G476A, H1*+G7A+D118T+S132L+W140Y+R180*+S181*+T182S+E189P+N194F+Y242F+E259G+M260D+F266Y+N279S+K280Y+G303R+E390A+G475K, H1*+G7A+D118T+W140Y+R180*+S181*+T182S+E189P+N194F+Y242F+E259G+M260D+F266Y+N279S+K280Y+S302T+E390A+G475K, H1*+G7A+D118T+W140Y+R180*+S181*+T182S+E189P+N194F+Y242F+E259G+M260W+F266Y+N279S+K280Y+G303R+E390A+G476A, H1*+G7A+D118T+W140Y+R180*+S181*+T182D+E189P+N194F+Y242F+E259G+F266Y+N279S+K280Y+S302T+E390A+G475K, H1*+G7A+D118T+W140Y+R180*+S181*+T182D+E189P+N194F+Y242F+E259G+M260D+F266Y+N279S+K280Y+S302T+E390A+G475K, H1*+N3D+G7A+D118T+W140Y+R180*+S181*+T182D+E189P+N194F+Y242F+E259G+F266Y+N279S+K280Y+S302T+E390A+H420A+G475K, H1*+G7A+R37N+D118T+W140Y+R180*+S181*+T182D+E189P+N194F+Y242F+E259G+F266Y+N279S+K280Y+S302Y+E390A+G475K, H1*+G7A+D118T+W140Y+R180*+S181*+T182S+A185Q+E189P+N194F+T224Y+Y242F+F266Y+N279S+K280Y+G303R+E390A+G476A, H1*+G7A+D118T+W140Y+R180*+S181*+T182S+A185Q+E189P+N194F+Y242F+E259G+F266Y+N279S+K280Y+S302T+E390A+G475K, H1*+G7A+D118T+W140Y+R180*+S181*+T182S+A185Q+E189P+N194F+Y242F+E259G+F266Y+N279S+K280Y+G303R+E390A+G476A, H1*+G7A+D118T+S132L+W140Y+R180*+S181*+T182S+A185Q+E189P+N194F+L205Y+Y242F+E259G+M260D+F266Y+N279S+K280Y+G303R+E390A+G475K, H1*+N3D+G7A+D118T+W140Y+R180*+S181*+T182S+A185Q+E189P+N194F+Y242F+E259G+F266Y+N279S+K280Y+S302T+E390A+H420A+G475K, H1*+G7A+D118T+W140Y+R180*+S181*+T182S+A185Q+E189P+N194F+Y242F+E259G+M260W+F266Y+N279S+K280Y+G303R+E390A+G476A, H1*+G7A+W140Y+R180*+S181*+E189P+N194F+Y242F+E259G+M260D+N279S+K280Y+S302T+E390A+G475K, H1*+N3D+G7A+W140Y+R180*+S181*+E189P+N194F+Y242F+E259G+F266Y+N279S+K280Y+S302T+E390A+H420A+G475K, H1*+G7A+R37N+W140Y+R180*+S181*+E189P+N194F+Y242F+E259G+F266Y+N279S+K280Y+S302Y+E390A+G475K, H1*+G7A+W140Y+R180*+S181*+E189P+N194F+Y242F+E259G+F266Y+N279S+K280Y+G303R+E390A+G476A, H1*+G7A+D118T+W140Y+R180*+S181*+T182S+E189P+N194F+Y242F+E259G+M260D+N279S+K280Y+S302Y+E390A+N470M+G475K, H1*+G7A+D118T+W140Y+R180*+S181*+T182D+E189P+N194F+Y242F+E259G+M260D+N279S+K280Y+S302Y+E390A+N470M+G475K, H1*+G7A+D118T+W140Y+R180*+S181*+T182D+E189P+N194F+Y242F+E259G+M260W+N279S+K280Y+G303R+E390A+G476A, H1*+G7A+D118T+W140Y+R180*+S181*+T182S+A185Q+E189P+N194F+Y242F+E259G+M260W+N279S+K280Y+G303R+E390A+G476A, H1*+G7A+D118T+W140Y+R180*+S181*+T182S+E189P+N194F+T224Y+Y242F+N279S+K280Y+G303R+E390A+G476A, H1*+G7A+D118T+W140Y+R180*+S181*+T182D+E189P+N194F+T224Y+Y242F+N279S+K280Y+G303R+E390A+G476A, H1*+G7A+D118T+W140Y+R180*+S181*+T182S+A185Q+E189P+N194F+T224Y+Y242F+N279S+K280Y+G303R+E390A+G476A, H1*+G7A+D118T+W140Y+R180*+S181*+T182S+A185Q+E189P+N194F+Y242F+E259G+N279S+K280Y+S302T+E390A+G475K, H1*+G7A+D118T+W140Y+R180*+S181*+T182D+E189P+N194F+Y242F+E259G+F266Y+N279S+K280Y+G303R+E390A+G476A, H1*+G7A+D118T+W140Y+R180*+S181*+T182D+E189P+N194F+Y242F+E259G+M260D+N279S+K280Y+S302Y+E390A+N470M+G475K, H1*+G7A+D118T+W140Y+R180*+S181*+T182D+A185Q+E189P+N194F+Y242F+E259G+F266Y+N279S+K280Y+G303R+E390A+G475K, H1*+G7A+D118T+W140Y+R180*+S181*+T182D+A185Q+Y242F+S243Q+E259G+F266Y+N279S+G303R+E390A+G475K, H1*+G7A+D118T+W140Y+R180*+S181*+T182D+N194F+Y242F+S243Q+E259G+F266Y+N279S+G303R+E390A+G475K, H1*+G7A+D118T+W140Y+R180*+S181*+T182S+A185Q+E189P+N196I+Y242F+E259G+F266Y+N279S+K280Y+G303R+E390A+G475K, H1*+G7A+D118T+W140Y+R180*+S181*+T182S+A185D+E189P+Y242F+E259G+F266Y+N279S+K280Y+G303R+E390A+H420Y+G475K, H1*+G7A+D118T+W140Y+R180*+S181*+A185Q+E189P+N194F+L205Y+Y242F+E259G+F266Y+N279S+K280Y+G303R+E390A+G475K, H1*+G7A+W140Y+R180*+S181*+E189P+N194F+Y242F+E259G+M260W+N279S+K280Y+G303R+E390A+G476A, H1*+G7A+D118T+W140Y+R180*+S181*+T182S+E189P+N194F+Y242F+E259G+M260D+N279S+K280Y+S302Y+E390A+N470M+G475K, H1*+G7A+D118T+W140Y+R180*+S181*+T182D+E189P+N194F+T224Y+Y242F+N279S+K280Y+G303R+E390A+G476A, H1*+G7A+D118T+W140Y+R180*+S181*+T182S+E189P+N194F+T224Y+Y242F+N279S+K280Y+G303R+E390A+G476A, H1*+G7A+D118T+W140Y+R180*+S181*+T182D+A185Q+E189P+N194F+T224Y+Y242F+N279S+K280Y+G303R+E390A+G476A, H1*+G7A+D118T+W140Y+R180*+S181*+T182D+A185Q+N194F+Y242F+E259G+N279S+K280Y+S302T+E390A+G475K, H1*+G7A+R37N+D118T+W140Y+R180*+S181*+T182S+A185Q+E189P+N194F+L205Y+Y242F+E259G+N279S+K280Y+S302Y+E390A+G475K, H1*+G7A+D118T+W140Y+R180*+S181*+T182S+A185Q+E189P+N194F+L205Y+Y242F+E259G+M260D+N279S+K280Y+S302T+E390A+G475K, H1*+N3D+G7A+D118T+W140Y+R180*+S181*+T182D+E189P+N194F+L205Y+Y242F+E259G+N279S+K280Y+S302T+E390A+H420A+G475K, H1*+G7A+W140Y+R180*+S181*+E189P+N194F+Y242F+E259G+M260D+N279S+K280Y+S302Y+E390A+N470A+G475K, H1*+G7A+S132L+W140Y+R180*+S181*+E189P+N194F+Y242F+E259G+M260D+N279S+K280Y+G303R+E390A+G475K, H1*+G7A+A31S+R37N+D118T+W140Y+R180*+S181*+T182S+A185Q+E189P+N194F+Y242F+E259G+F266Y+N279S+K280Y+S302Y+E390A+G475K, H1*+G7A+D118T+W140Y+R180*+S181*+T182S+A185Q+E189P+N194F+Y242F+E259G+M260W+N279S+K280Y+G303R+E390A+G476A, H1*+G7A+D118T+W140Y+R180*+S181*+T182D+E189P+N194F+L205Y+T224Y+Y242F+F266Y+N279S+K280Y+G303R+E390A+G476A, H1*+G7A+D118T+W140Y+R180*+S181*+T182S+A185Q+E189P+N194F+Y242F+E259G+N279S+K280Y+S302T+E390A+G475K, R180*+S181*, T40N+F116L+D123N+N126D+N128Y+T131I+F155W+R180*+S181*+T182D+E189P+F202Y+K457N+P458S+G475K+G476E, A60V+Q98L+G110K+R180*+S181*+A383D, Q98L+G110K+S132D+Y135E+R180*+S181*+T315A, G7H+Q98L+G110K+R180*+S181*, G7H+W167P+R180*+S181*+G455R, Q98L+V103A+G110K+R180*+S181*, T114K+R180*+S181*, E169V+R180*+S181*, R180*+S181*+W188C, W167D+R180*+S181*, W167S+R180*+S181*, W167E+R180*+S181*, W167P+R180*+S181*, R180*+S181*+E193I+H323N, R180*+S181*+A203F, W159L+R180*+S181*, W159H+R180*+S181*, K178A+R180*+S181*, A111 L+R180*+S181*, G110K+R180*+S181*, and A111G+R180*+S181*, using SEQ ID NO: 1 for numbering and wherein said variant has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to SEQ ID NO: 1, 2, 3, 4, 5, 6, 7, 8, 9, 10, 11, 12, 13 or 14 and wherein the variant has alpha-amylase activity.

8. The variant according to claim 1, wherein said parent alpha-amylase is selected from the amino acid sequences set forth in SEQ ID NO: 1, 2, 3, 4, 5, 6, 7, 8, 9, 10, 11, 12, 13, 14, or any alpha-amylase has at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity sequence identity to any of the amino acid sequences set forth in SEQ ID NOs: 1, 2 or 3.

9. The variant according to claim 1, wherein said parent alpha-amylase comprises or consists of the amino acid sequence set forth in SEQ ID NO: 1, SEQ ID NO: 2 and SEQ ID NO: 3.

10. A method of obtaining an alpha-amylase variant of a parent alpha-amylase by introducing a modification at one or more positions corresponding to positions: 1, 2, 3, 7, 26, 31, 37, 40, 41, 54, 56, 60, 63, 72, 73, 93, 94, 98, 103, 104, 105, 106, 109, 110, 111, 113, 114, 116, 117, 118, 123, 126, 128, 131, 132, 134, 135, 136, 137, 138, 140, 142, 144, 146, 151, 152, 155, 159, 160, 165, 167, 169, 172, 173, 174, 176, 177, 178, 180, 181, 182, 183, 185, 188, 189, 193, 194, 196, 202, 203, 205, 224, 225, 242, 243, 244, 252, 256, 258, 259, 260, 261, 266, 276, 279, 280, 283, 286, 302, 303, 315, 320, 321, 322, 323, 346, 354, 359, 360, 361, 362, 365, 371, 383, 390, 394, 395, 405, 409, 414, 420, 422, 427, 455, 457, 458, 470, 472, 473, 474, 475 and 476, using SEQ ID NO: 1 for numbering, and wherein the said variant has 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the SEQ ID NO: 1, 2, 3, 4, 5, 6, 7, 8, 9, 10, 11, 12, 13 or 14.

11. A method of improving wash performance of an alpha-amylase variant of a parent alpha-amylase, said variant having the amino acid sequence of SEQ ID NO: 1, or having at least 60% sequence identity thereto, said method comprising the steps of: (a) introducing a substitution and/or deletion of two, three or four positions in the parent alpha-amylase said positions corresponding to position 180, 181, 182, and 183 using SEQ ID NO: 1 for numbering; and (b) introducing a modification at one or more positions corresponding to positions H1*, H2, N3D, N3G, N3S, G7A, G7H, G7S, R26Y, A31S, R37A, R37N, R37V, T40G, T40N, A41D, N54S, V56T, A60V, A60P, L63F, K72R, G73L, K93R, N94D, Q98L, V103A, V103I, V103L, V104M, M105I, M105L, N106D, A109G, G110K, A111G, A111L, G113Q, G113S, G113A, T114K, F116L, F116N, F116Q, F116W, V117G, D118Q, D118T, D123N, N126D, N128Y, T131I, T131V, S132D, S132L, T134D, Y135E, Q136T, Q136K, I137F, Q138V, W140S, W140M, W140Y, K142E, K142G, K142P, D144N, P146S, P146H, T151A, Y152L, F155W, W159H, W159L, Y160P, T165V, W167A, W167D, W167E, W167F, W167P, W167S, E169I, E169Q, E169V, K172G, K172Q, L173F, N174K, N174S, I176V, Y177D, K178A, A185D, A185E, A185K, A185N, A185Q, A185R, W188C, E189P, E193I, N194F, F202Y, F202Y, A203F, L205F, L205N, L205Q, L205R, L205V, L205Y, T224F, T224Y, N225S, Y242F, Y242L, S243Q, F244I, F244L, V252I, T256A, T256D, K258V, E259G, E259V, M260D, M260W, F261K, F261P, F261S, F266Y, N276F, N276W, N276Y, N279S, N280F, N280W, N280Y, W283N, S286K, S286L, S302T, S302Y, G303R, T315A, H320T, P321H, P321Y, M322G, M322Y, H323A, H323N, H323W, W346N, L354V, E359W, Q360M, Q360W, Q360Y, G361M, Y362V, V365I, Y371S, Y371N, A383D, E390A, E390D, N394W, N394D, F395Y, D405G, I409L, R414M, H420P, H420A, H420Q, H420Y, N422V, T427L, G455R, K457N, K457R, R458S, N470A, N470M, S472R, V473C, N474F, N474L, G475E, G475K, G476H, G476E and G476A, using SEQ ID NO: 1 for numbering, and wherein the said variant has 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99%, but less than 100% sequence identity to the SEQ ID NO: 1, 2, 3, 4, 5, 6, 7, 8, 9, 10, 11, 12, 13 or 14 and wherein said variant has alpha-amylase activity and improved wash performance to said parent polypeptide.

12. (canceled)

13. A composition comprising the variant according to claim 1.

14. The composition according to claim 13, wherein the composition is selected from a group comprising: liquid detergents, solid detergents, gel detergents, powder detergents, tabs, pods, pouch and granule detergents.

15. The composition according to claim 13, which is a liquid laundry or liquid dish wash composition, such as an Automatic Dish Wash (ADW) liquid detergent composition, or a solid laundry, such as a soap bar, or powder dish wash composition, such as an ADW unit dose detergent composition.
